(19) United States
(12) Patent Application Publication
(10) Pub. No.: US 2024/0425523 A1
(43) Pub. Date: Dec. 26, 2024

(54) Compounds for the Degradation of EGFR Kinase

(71) Applicant: Beigene Switzerland GMBH, Basel (CH)

(72) Inventors: Huaqing Liu, Beijing (CN); Bailin Lei, Beijing (CN); Yizhou Zhao, Beijing (CN); Xinzhu Qi, Beijing (CN); Guanjun Liu, Beijing (CN); Zhiwei Wang, Beijing (CN)

(21) Appl. No.: 18/678,898

(22) Filed: May 30, 2024

(30) Foreign Application Priority Data
May 31, 2023 (WO) PCT/CN2023/097387
Oct. 19, 2023 (WO) PCT/CN2023/125498
Apr. 30, 2024 (WO) PCT/CN2024/090756

(51) Int. Cl. C07D 519/00; A61K 31/4545; A61K 31/496; A61K 31/499; A61K 31/4995; C07D 498/22

(52) U.S. Cl. CPC C07D 519/00; A61K 31/4545; A61K 31/496; A61K 31/499; A61K 31/4995; C07D 498/22

(57) ABSTRACT

Disclosed herein are novel bifunctional compounds formed by conjugating EGFR inhibitor moieties with E3 ligase ligand moieties, methods of preparation, and uses thereof.

### CROSS REFERENCE TO RELATED APPLICATIONS

This application claims priority to International Application No. PCT/CN2023/097387, filed May 31, 2023, and to International Application No. PCT/CN2023/125498, filed Oct. 19, 2023, and to International Application No. PCT/CN2024/090756, filed Apr. 30, 2024, the disclosures of each of which are hereby incorporated by reference in their entireties.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9; Sakamoto K. M. et al., Methods Enzymol. 2005; 399:833-847). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987). As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) PROTACs as useful modulators promoting the selective degradation of a wide range of target proteins have been reported in antitumor studies. (Lu J. et al., Chem Biol. 2015; 22(6):755-763; Ottis P. et al., Chem Biol. 2017; 12(4):892-898; Crews C. M. et al., J Med Chem. 2018; 61(2):403-404; Neklesa T. K. et al., Pharmacol Ther 2017, 174:138-144; Cermakova K. et al., Molecules, 2018.23(8); An S. et al., EBioMedicine, 2018; Lebraud H. et al., Essays Biochem. 2017; 61(5): 517-527; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, WO2018071606, WO2021178920, WO2021127283, WO2021127190, WO202111871 and WO202111913.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562). Thus, there is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562, US20190106417, WO202157882, WO2021123087, WO2021133809, WO2021168074, WO2021208918 and WO2021216440.

A number of EGFR-targeting PROTACs which were designed to degrade EGFR mutant proteins have been published (Zhang X., et al. Eur J. Med. Chem. 2020, 192, 112199; Zhang H, et al. Eur J. Med. Chem. 2020, 189, 112061; Lu X, Med. Res. Rev. 2018, 38(5):1550-1581. He K., et al. Bioorg. Med. Chem. Lett. 2020, 15, 127167). Most of these published molecules are based on first, second, and third generation of EGFR inhibitors (WO2021023233, WO2019121562 and WO2018119441) or allosteric EGFR inhibitors (WO2021127561). However, there were no data which showed those EGFR-Targeting PROTACs degrading all the main EGFR mutations, such as Del19, L858R, Del19/T790M, L858R/T790M, Del19/T790M/C797S, L858R/T790M/C797S.

The present application provides novel bifunctional compounds and compositions for the treatment of serious diseases affected by EGFR modulation, especially for the treatment of cancer, preferably selected from pancreatic cancer, breast cancer, glioblastoma multiforme, head and neck cancer, and non-small cell lung cancer.

### SUMMARY OF THE INVENTION

One objective of the present disclosure is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

The compounds described herein, or salts thereof, are useful in the treatment of a disease that can be affected by EGFR modulation. The present disclosure provides the use of the compounds described herein or pharmaceutically acceptable salts thereof, in the manufacture of a medicament for the treatment of a disease that can be affected by EGFR modulation. The present disclosure further provides a compound described herein or a pharmaceutically acceptable salt thereof, for use in the treatment of a disease that can be affected by EGFR modulation. The present application further provides a method of treating a proliferative disorder, comprising administering to a subject in need thereof a therapeutically effective amount of the compounds described herein or a pharmaceutically acceptable salt thereof.

Aspect 1. A compound of Formula (I):

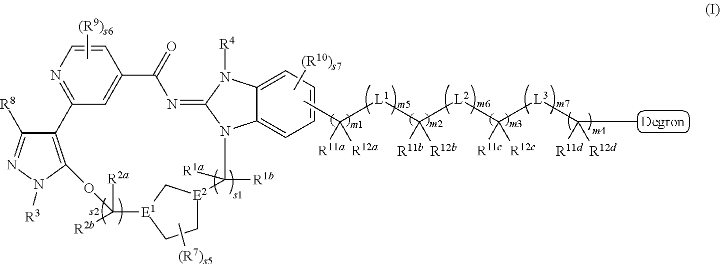

or an N-oxide, pharmaceutically acceptable salt, stereoisomer, tautomer, deuterated analog, or a prodrug thereof,
wherein:
E¹ is N or CR⁵;
E² is N or CR⁶;
R^{1a}, R^{1b}, R^{2a} and R^{2b} are each independently absent, hydrogen, halogen, —C₁₋₈alkyl, —C₂₋₈alkenyl, —C₂₋₈alkynyl, —C₁₋₈alkoxy, —C₃₋₈cycloalkyl or —CN; wherein each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl and —CN;
R³ and R⁴ are each independently hydrogen, —C₁₋₆alkyl, or —C₃₋₈cycloalkyl; wherein each said —C₁₋₆alkyl or —C₃₋₈cycloalkyl is optionally substituted with at least one substituent selected from hydrogen, hydroxy, halogen, and —C₁₋₆alkoxy;
R⁵ and R⁶ are each independently absent, hydrogen, halogen, —C₁₋₈alkyl, —C₂₋₈alkenyl, —C₂₋₈alkynyl, —C₁₋₈alkoxy, —C₃₋₈cycloalkyl or —CN; wherein each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈cycloalkyl and —CN; or
R⁵ and R⁶ with the carbon atoms to which they are attached, form a 3- to 12-membered ring, said ring comprising 0-3 heteroatoms independently selected from nitrogen, oxygen or sulfur; wherein said ring is optionally substituted with at least one substituent selected from halogen, hydroxy, and —C₁-C₈alkyl; R⁷ is each independently hydrogen, halogen, —C₁₋₈alkyl, —C₂₋₈alkenyl, —C₂₋₈alkynyl, —C₁₋₈alkoxy, —C₃₋₈cycloalkyl or —CN; wherein each said —C₁₋₈alkyl, —C₂₋₈alkenyl, —C₂₋₈alkynyl, —C₁₋₈alkoxy, or —C₃₋₈cycloalkyl is optionally substituted with at least one substituent selected from hydrogen, halogen, —C₁₋₈alkoxy, —C₃₋₈ cycloalkyl and —CN; or
two R⁷ with the carbon atom(s) to which they are attached, form a 3- to 12-membered ring, said ring comprising 0-3 heteroatoms independently selected from nitrogen, oxygen or sulfur; wherein said ring is optionally substituted with at least one substituent selected from halogen, hydroxy, and —C₁-C₈alkyl;
R⁸ and R⁹ are each independently selected from hydrogen, halogen, —C₁-C₆alkyl and C₃-C₈cycloalkyl; wherein each of —C₁-C₆alkyl or C₃-C₈cycloalkyl is optionally substituted with at least one substituent selected from hydrogen, halogen, hydroxy, and —C₁₋₆alkoxy;
R¹⁰ is each independently selected from hydrogen, halogen, —C₁-C₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b}, —NR^{10a}SO₂R^{10b}, and —CN;
wherein each of —C₁-C₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl is optionally substituted with at least one R^{10c};
R^{10a} and R^{10b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, and 5- to 12-membered heteroaryl; wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{10d};
R^{10c} and R^{10d} are each independently selected from halogen, hydrogen, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{10e}R^{10f}, —OR^{10e}, —SR^{10e}, —SO₂R^{10e}, —SO₂NR^{10e}R^{10f}, —C(O)R^{10e}, —CO₂R^{10e}, —C(O)NR^{10e}R^{10f}, —NR^{10e}COR^{10f}, —NR^{10e}CO₂R^{8f}, —NR^{10e}SO₂R^{10f}, and —CN;
R^{10e} and R^{10f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R^{11a}, R^{11b}, R^{11c}, R^{11d}, R^{12a}, R^{12b}, R^{12c} and R^{12d} are each independently absent, oxo, hydrogen, halogen, —C₁₋₈alkyl, —C₂₋₈alkenyl, —C₂₋₈alkynyl, —C₈alkoxy, or —C₃₋₈cycloalkyl; wherein each of said —C₁₋₈alkyl, —C₂₋₈alkenyl, —C₂₋₈alkynyl, —C₁₋₈alkoxy or —C₃₋₈cycloalkyl is optionally substituted with at least one substituent selected from hydrogen, halogen, —C₁₋₈alkyl, —C₂₋₈alkenyl, —C₂₋₈alkynyl, —C₁₋₈alkoxy, and —CN;
L¹ is selected from —O—, —NR^{a}—, —C(O)—, *^{L1}—C(O)NR^{a}—**^{L1}, *^{L1}—C(O)O—**^{L1}, *^{L1}—NR^{a}C(O)—**^{L1}, *^{L1}—OC(O)—**^{L1},

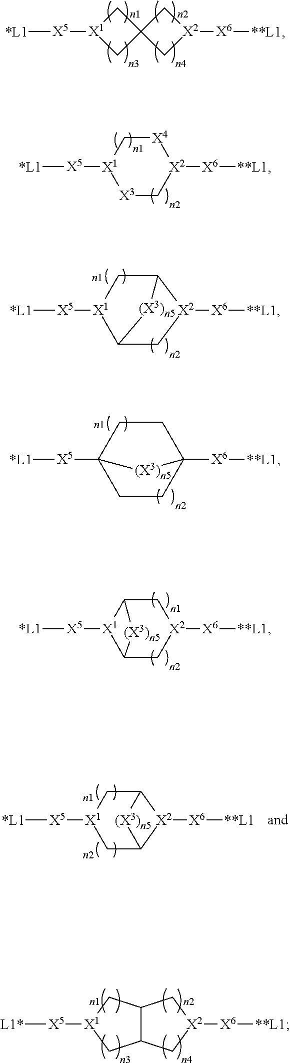

wherein each of said

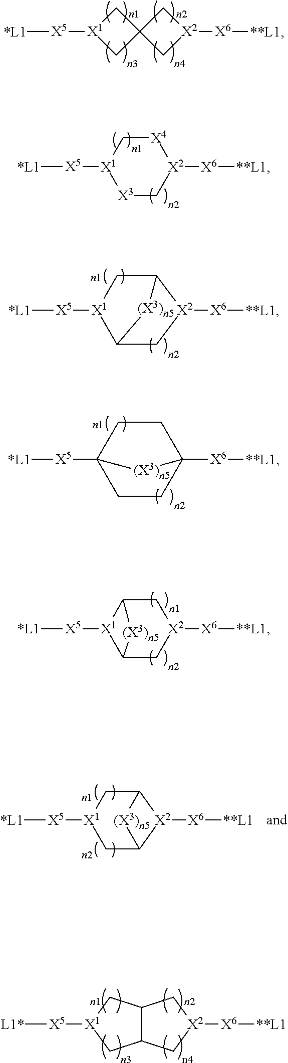

is optionally substituted with at least one R^{L1c};
wherein *^{L1} refers to the position attached to the

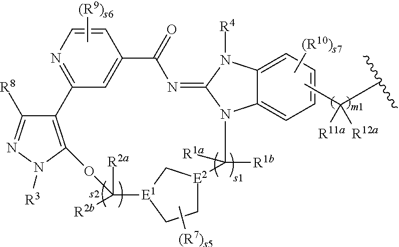

moiety, and
**^{L1} refers to the position attached to the

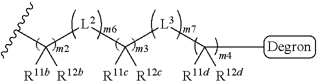

moiety; L² is selected from —O—, —NR^{a}—, —C(O)—, *^{L2}—C(O)NR^{a}—**^{L2}, *^{L2}—C(O)O—**^{L2}, *^{L2}—NR^{a}C(O)—**^{L2}, *^{L2}—OC(O)—**^{L2},

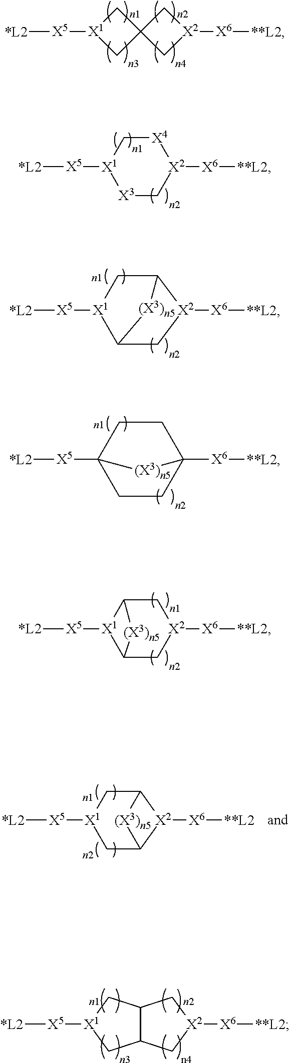

wherein each of said

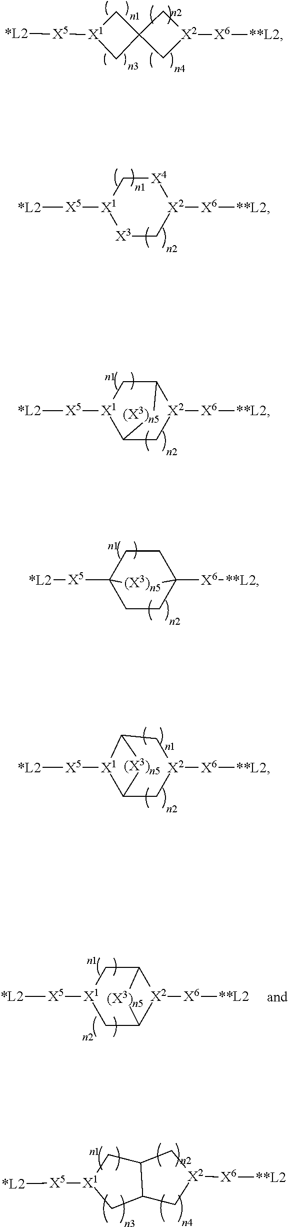

is optionally substituted with at least one R^{L2c};
wherein *^{L2} refers to the position attached to the

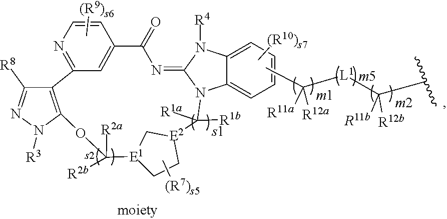

and **^{L2} refers to the position attached to the

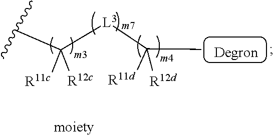

L³ is selected from —O—, —NR^{a}—, —C(O)—, *^{L3}—C(O)NR^{a}—**^{L3}, *^{L3}—C(O)—**^{L3}, *^{L3}—NR^{a}C(O)—**^{L3}, *^{L3}—OC(O)—**^{L3},

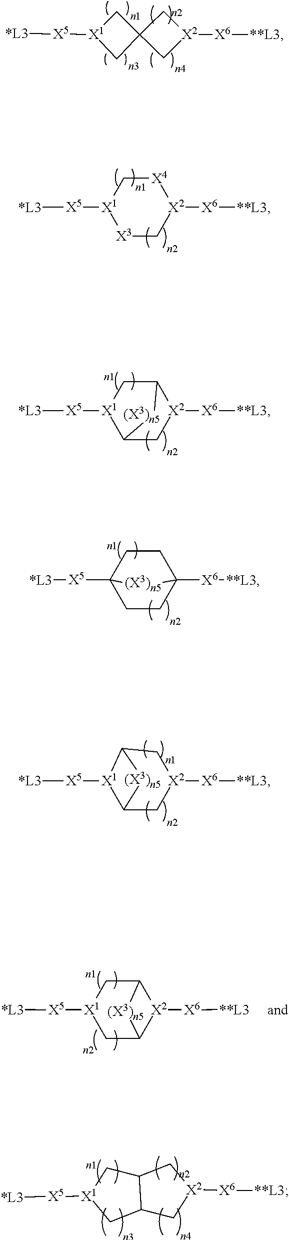

wherein each of said

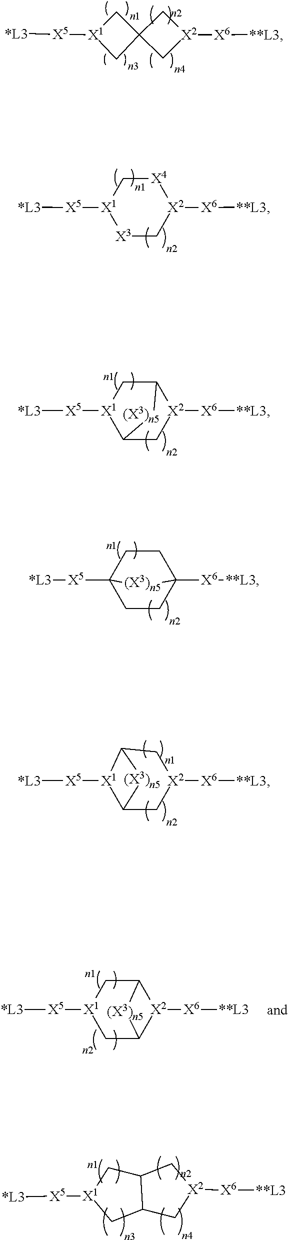

is optionally substituted with at least one R^{L3c};
wherein *^{L3} refers to the position attached to the

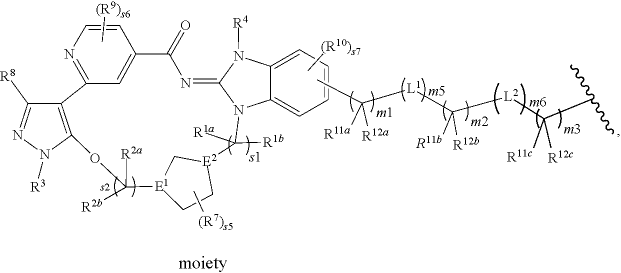

and **^{L3} refers to the position attached to the

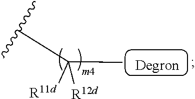

each of said R^{L1c}, R^{L2c} and R^{L3c} are independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl; wherein each of said —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl is optionally substituted with at least one R^{Lca},
R^{Lca} is independently absent, oxo (═O), halogen, hydroxy, —CN, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen and sulfur; said ring is optionally substituted with at least one substituent selected from halogen, hydroxy, and —C₁-C₈alkyl;
two R^{L2c} together with the atoms to which they are attached, form a 3- to 12-membered ring, said ring comprising 0-3 heteroatoms independently selected from nitrogen, oxygen and sulfur; said ring is optionally substituted with at least one substituent selected from halogen, hydroxy, and —C₁-C₈alkyl;
two R^{L3c} together with the atoms to which they are attached, form a 3- to 12-membered ring, said ring comprising 0-3 heteroatoms independently selected from nitrogen, oxygen and sulfur; said ring is optionally substituted with at least one substituent selected from halogen, hydroxy, and —C₁-C₈alkyl;
[symbol] is selected from

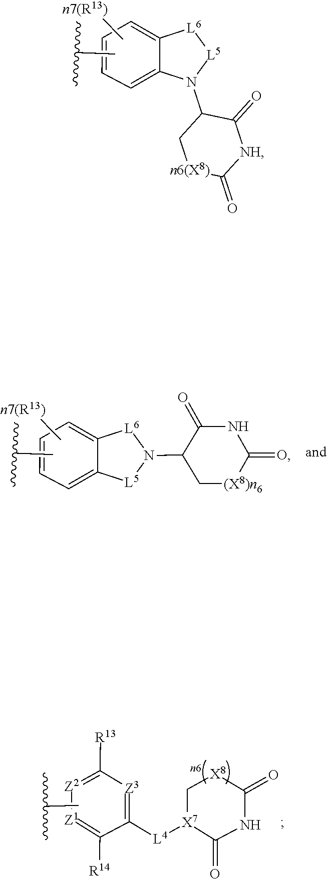

Z¹, Z² and Z³ are each independently N or CR^{z}, provided that Z¹, Z² and Z³ are not N at the same time;
R^{z}, at each occurrence, is independently absent, hydrogen, halogen, —C₁₋₈alkyl, —NR^{Za}R^{Zb}, —OR^{Za}, —SR^{Za}, C₃-C₈cycloalkyl, 3- to 8-membered heterocyclyl, or CN; wherein each of —C₁₋₈alkyl, C₃-C₈cycloalkyl, 3- to 8-membered heterocyclyl is optionally substituted with at least one R^{Zc};
wherein the

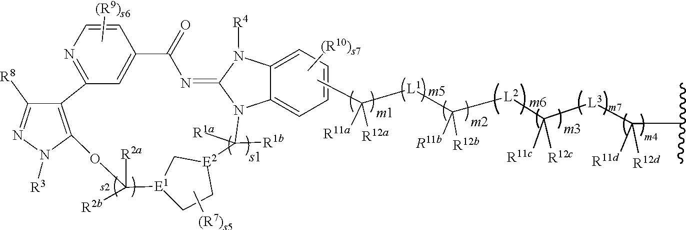

moiety is linked to the

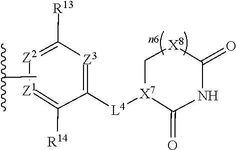

moiety via any one of Z¹ or Z² which is CR^{z} and R^{z} is absenct;
R^{Za} and R^{Zb} are each independently absent, hydrogen, —C₁-C₈alkyl, C₃-C₈cycloalkyl, 3- to 8-membered heterocyclyl, C₆-C₁₂aryl, or 5- to 12-membered heteroaryl; wherein each of said —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, —C₁₋₈alkoxy, C₃-C₈cycloalkyl, 3- to 8-membered heterocyclyl, C₆-C₁₂aryl, or 5- to 12-membered heteroaryl;
R¹³ and R¹⁴ are each independently absent, hydrogen, halogen, —C₁₋₈alkyl, —C₂₋₈alkenyl, —C₂₋₈alkynyl, —C₁₋₈ alkoxy, —C₃-C₈cycloalkyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, or —NR^{13a}SO₂R^{13b}; wherein each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13d}, —NR^{13c}R^{13d}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently absent, hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl;
L⁴, L⁵ and L⁶ are each independently absent, a single bond, —O—, —NR^{a}—, —(CR^{a}R^{b})ₙ₈—, —O(CR^{a}R^{b})ₙ₈—, —NR^{a}(CR^{a}R^{b})ₙ₈— or —C(O)—;
at each occurrence, X¹, X² and X⁷ are each independently —CR^{a}, or N;
at each occurrence, X³, X⁴ and X⁸ are each independently —NR^{a}—, —O—, —S— or —CR^{a}R^{b}—;
at each occurrence, X⁵ and X⁶ are each independently absent, a single bond, —C(O)—, —NR^{a}— or —O—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl and 5- to 12-membered heteroaryl; wherein each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent selected from halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl and 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen and sulfur; wherein said ring is optionally substituted with at least one substituent selected from halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl and 5- to 12-membered heteroaryl;
m1, m2, m3 and m4 are each independently 0, 1 or 2; provided that m1+m2+m3+m4≤4; m5, m6 and m7 are each independently 0, 1 or 2; provided that m5+m6+m7≥1;
n1, n2, n3, n4 and n5 are each independently 0, 1, 2 or 3;
n6 is each independently 0, 1, 2, 3 or 4;
n7 and n8 are each independently 0, 1, 2 or 3;
s1 and s2 are each independently 0, 1, 2 or 3;
s5, s6 and s7 are each independently 0, 1, 2 or 3;
provided that:
for any one of L¹, L² or L³, when X¹ is N, X⁵ is single bond, absent, or —C(O)—; and/or when X² is N, X⁶ is single bond, absent, or —C(O)—;
when L¹ is

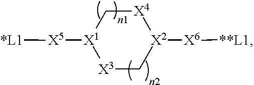

when X¹ is N, then X⁵ is a single bond, absent, or —C(O)—, X³ is —CR^{a}R^{b}—; when X² is N, X⁶ is single bond, absent, or —C(O)—, X⁴ is —CR^{a}R^{b}—;
when L² is

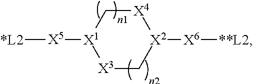

when X¹ is N, then X⁵ is a single bond, absent, or —C(O)—, X³ is —C^{a}R^{b}—; when X² is N, then X⁶ is a single bond, absent, or —C(O)—, X⁴ is —CR^{a}R^{b}—;
when L³ is

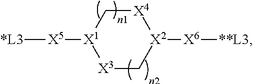

when X¹ is N, X⁵ is a single bond, absent, or —C(O)—, and/or X³ is —CR^{a}R^{b}—; when X² is N, X⁶ is a single bond, absent, or —C(O)—, and/or X⁴ is —CR^{a}R^{b}—.

Aspect 2. The compound of aspect 1, wherein the compound is selected from compounds of formula (IIa),

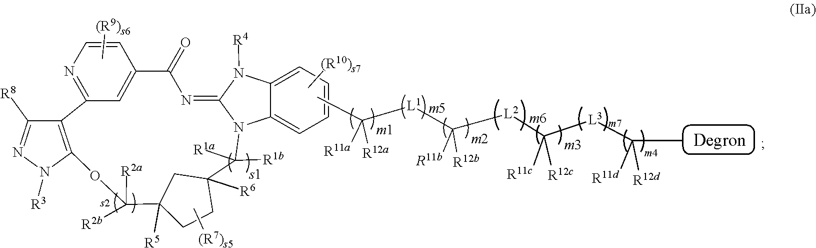

or an N-oxide, pharmaceutically acceptable salt, stereoisomer, tautomer, deuterated analog, or a prodrug thereof wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{11b}, R^{11c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIb):

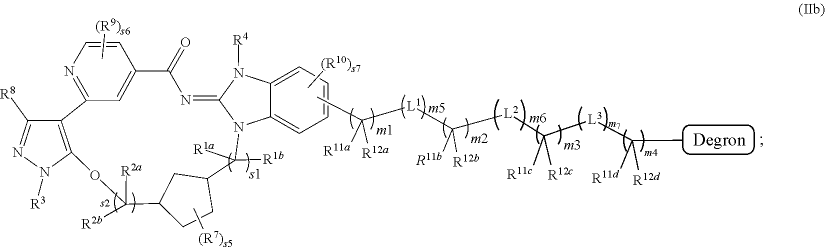

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIc):

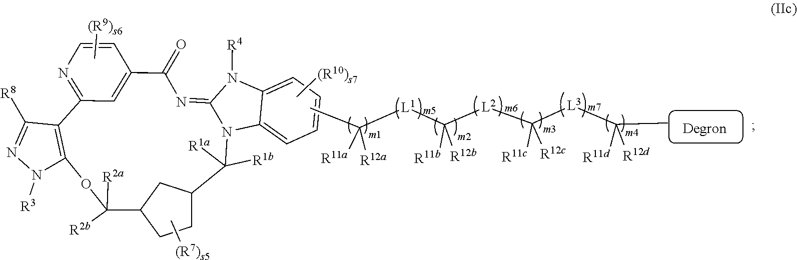

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IId):

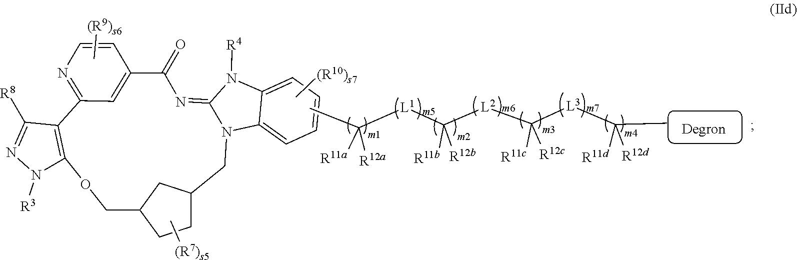

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIe):

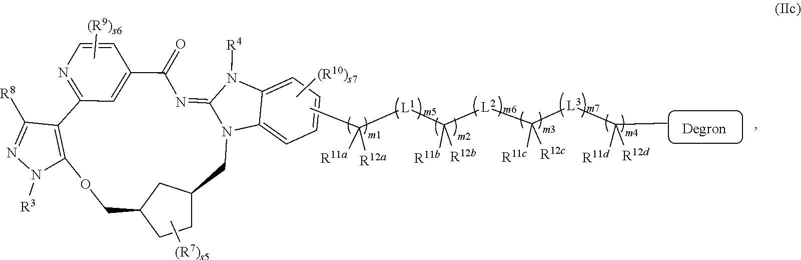

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIf):

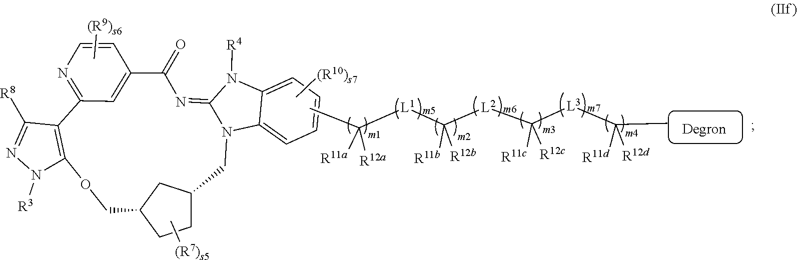

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIg):

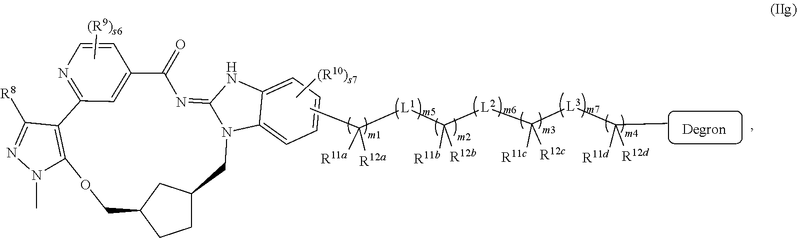

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIh):

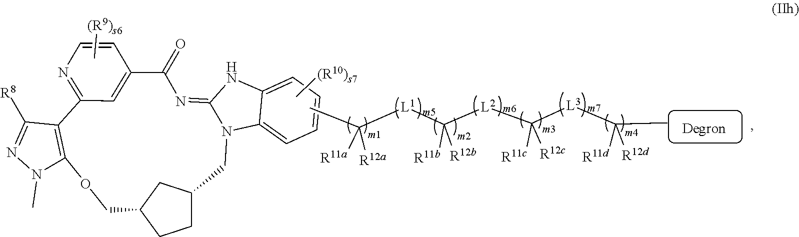

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIi):

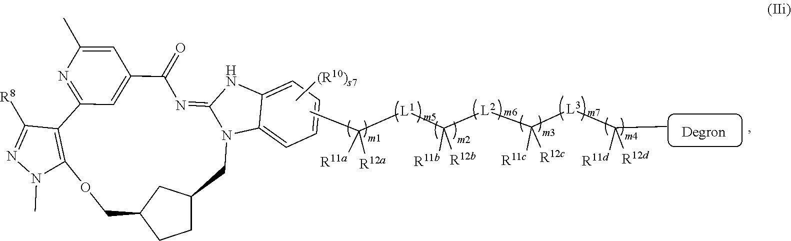

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{1b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIj):

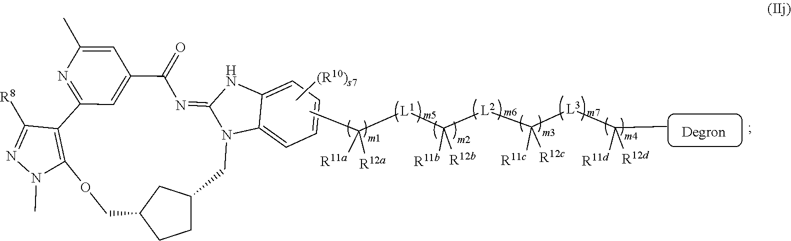

or an N-oxide, pharmaceutically acceptable salt, stereoisomer, tautomer, deuterated analog, or a prodrug thereof,
wherein, R^{1a}, R^{1b}R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

Aspect 3. The compound of aspect 1, wherein the compound is selected from compounds of formula (IIIa):

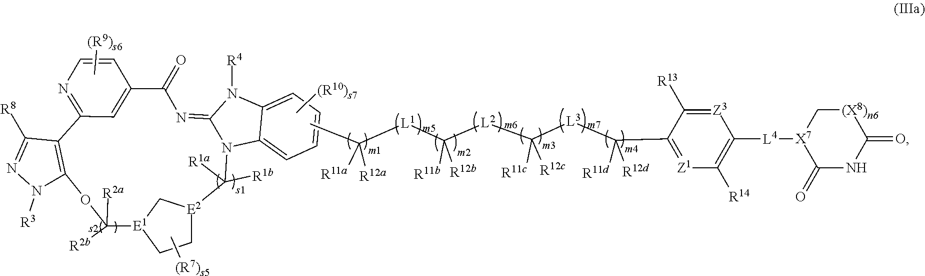

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R¹², L¹, L², L³, s1, s2, s5, s6, s7, m1, m2, m3, m4, m5, m6, m7 and Degron are as defined in aspect 1.

In one embodiment, the compound is selected from compounds of formula (IIIb):

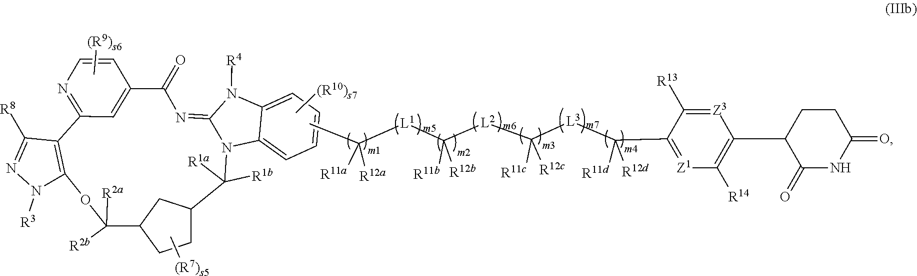

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIc):

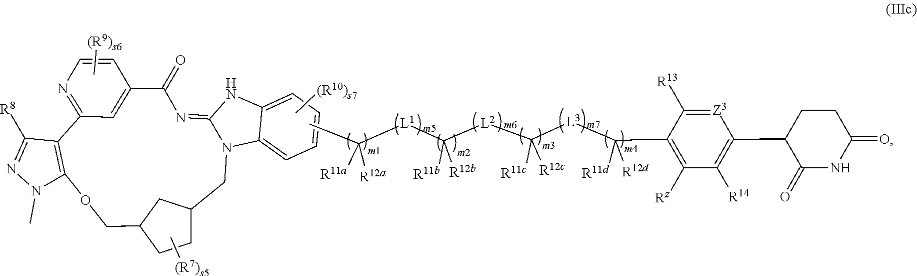

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIId):

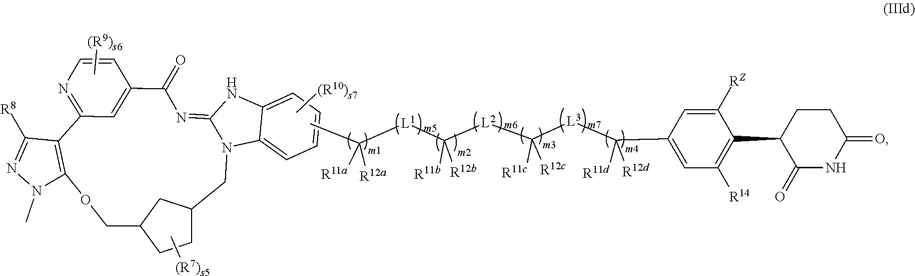

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIe):

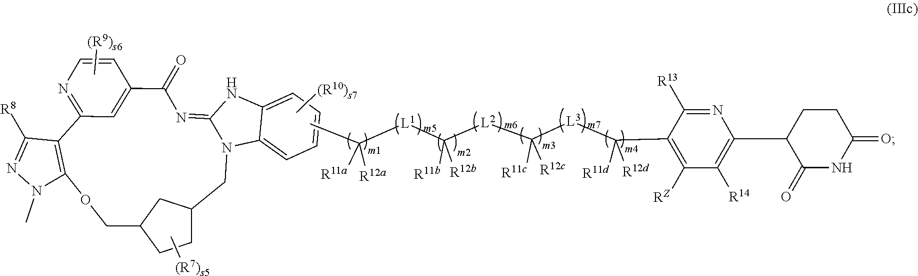

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIf):

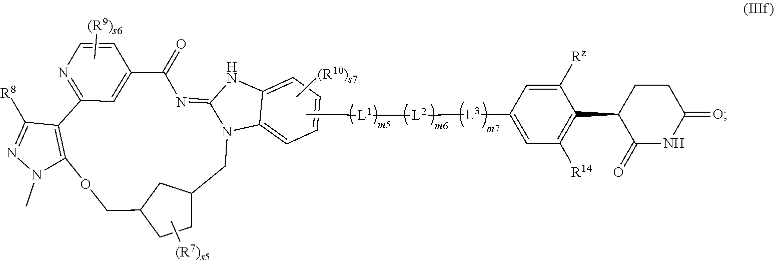

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIg):

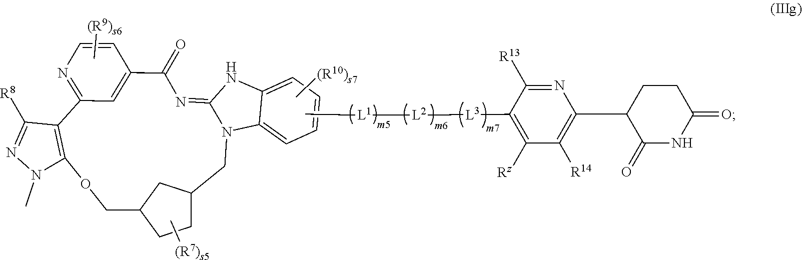

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIh):

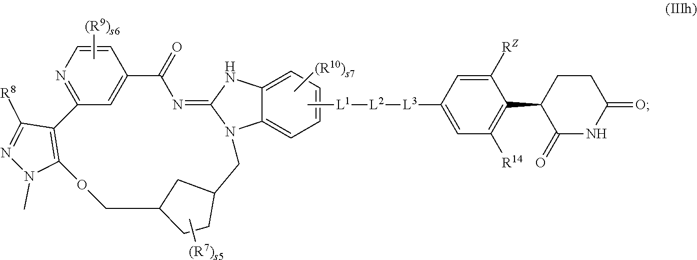

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIi):

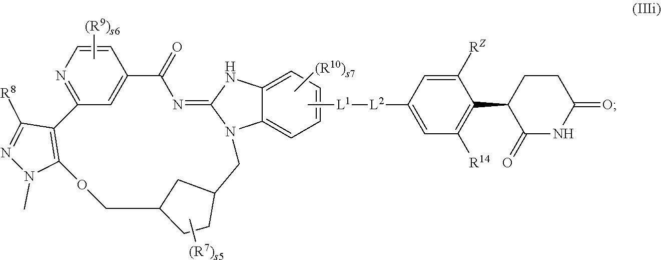

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from compounds of formula (IIIj):

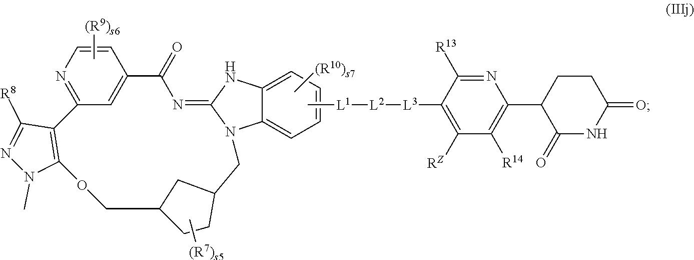

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from the compounds of formula (IIIk):

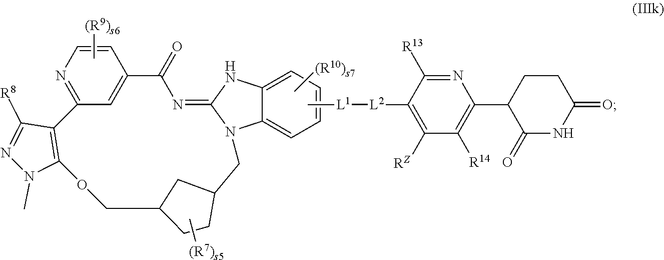

or an N-oxide, pharmaceutically acceptable salt, stereoisomer, tautomer, deuterated analog, or a prodrug thereof, wherein, R^{1a}, R^{1b}, R^{2a}, R^{2b}, R³, R⁴, R⁵, R⁶, R⁷, R⁸, R⁹, R¹⁰, R^{11a}, R^{11b}, R^{11c}, R^{11d}, R^{12a}, R^{12b}, R^{12c}, R^{12d}, L¹, L², L³, s1, s2, s5, s6, s7, m1, m2, m3, m4, m5, m6, m7 and Degron are as defined in aspect 1.

In one embodiment, the compound is selected from the compounds of formula (IIIm):

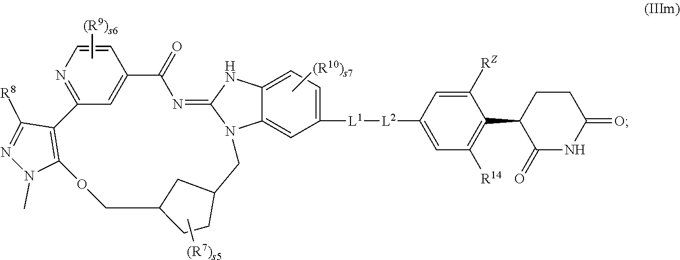

wherein
R⁷ is each independently absence, hydrogen, halogen or —C₁₋₃alkyl;
R⁸ and R⁹ are each independently selected from hydrogen, halogen, and —C₁-C₃alkyl;
R¹⁰ is each independently selected from hydrogen, halogen, or —C₁-C₃alkyl;
L¹ is selected from and

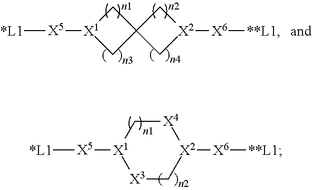

wherein each of said

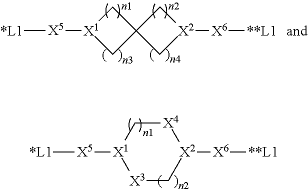

is optionally substituted with at least one R^{L1c};
wherein *^{L1} refers to the position attached to the

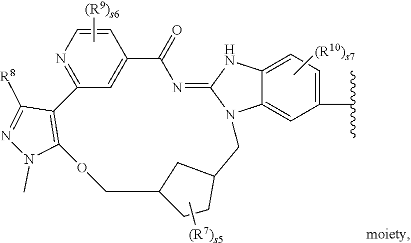

and
**^{L1} refers to the position attached to the

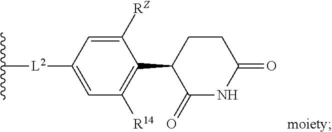

L² is selected from

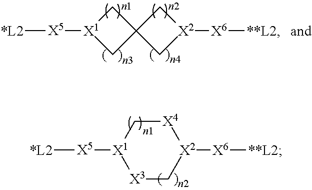

wherein each of said

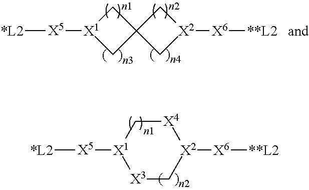

is optionally substituted with at least one R^{L2c};
wherein *^{L2} refers to the position attached to the

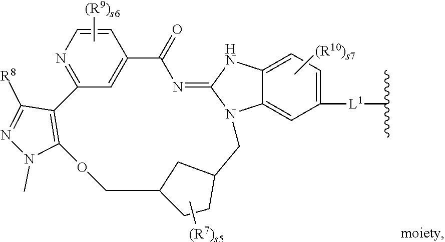

and
**^{L2} refers to the position attached to the

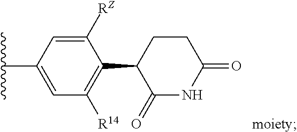

each of said R^{L1c} and R^{L2c} are independently absent, oxo (═O), halogen, hydroxy, —C₁-C₃alkyl or —C₁-C₃alkoxy; each of said —C₁-C₃alkyl and —C₁-C₃alkoxy is optionally substituted with at least one R^{Lca}, R^{Lca} is each independently absent, halogen, hydroxy, or —C₁-C₃alkoxy; or
two R^{L1c} together with the atoms to which they are attached, form a 3- to 5-membered ring, said ring comprising 0-1 heteroatoms independently selected from nitrogen and oxygen;
two R^{L2c} together with the atoms to which they are attached, form a 3- to 5-membered ring, said ring comprising 0-1 heteroatoms independently selected from nitrogen and oxygen;
R^{z}, at each occurrence, is independently absent, hydrogen, halogen, —C₁₋₃alkyl or —C₁-C₃alkoxy;
R¹⁴, at each occurrence, is independently absent, hydrogen, halogen, —C₁₋₃alkyl or —C₁-C₃alkoxy;
at each occurrence, X¹ and X² are each independently —CH or N;
at each occurrence, X³ and X⁴ are each independently —NH— and —CH₂—;
at each occurrence, X⁵ and X⁶ are each independently absent, a single bond, —C(O)— or —NR^{a}—
at each occurrence, R^{a} is independently selected from hydrogen, —C₁-C₃alkyl;
n1, n2, n3 and n4 are each independently 0, 1, or 2;
s5, s6 and s7 are each independently 0, 1, or 2.

Aspect 4. The compound of any one of the preceding aspects, wherein m1+m2+m3+m4≤3.

Aspect 5. The compound of any one of the preceding aspects, wherein m1+m2+m3+m4=0, 1, 2, or 3. In one embodiment, m1+m2+m3+m4=0, 1 or 2. In one embodiment, m1+m2+m3+m4=0. In one embodiment, m1+m2+m3+m4=1. In one embodiment, m1+m2+m3+m4=2.

Aspect 6. The compound of any one of the preceding aspects, wherein the total number of —CH₂— groups in the

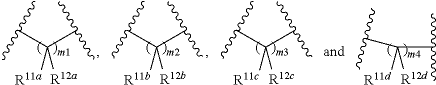

moieties combined is no more than 4,

In one embodiment, the total number of —CH₂— groups in the

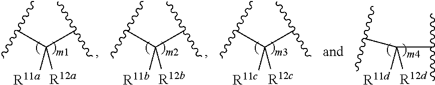

moieties combined is no more than 3,

In one embodiment, the total number of —CH₂— groups in the

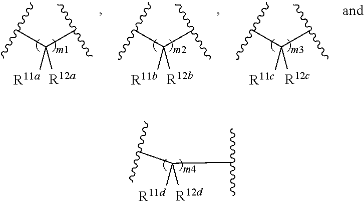

moieties combined is no more than 2.

Aspect 7. The compound of any one of the preceding aspects, wherein R³ and R⁴ are each independently hydrogen, methyl, ethyl, propyl, butyl, pentyl, hexyl, cyclopropyl, cyclobutyl, cyclopentyl, cyclohexyl, cycloheptyl or cyclooctyl; said methyl, ethyl, propyl, butyl, pentyl, hexyl, cyclopropyl, cyclobutyl, cyclopentyl, cyclohexyl, cycloheptyl or cyclooctyl is optionally substituted with at least one substituent selected from hydrogen, hydroxyl, F, Cl, Br, I, methoxy, ethoxy, propoxy, butoxy, pentoxy or hexoxy.

In one embodiment, R³ is —C₁₋₃alkyl, wherein said —C₁₋₃alkyl or —C₃₋₈cycloalkyl is optionally substituted with at least one substituent selected from hydrogen, hydroxy, halogen, —C₁₋₆alkoxy. In one embodiment, R³ is —C₁₋₃alkyl. In one embodiment, R⁴ is hydrogen In one embodiment, R³ and R⁴ are each independently hydrogen, methyl, ethyl, propyl, butyl, pentyl, hexyl. In one embodiment, R³ and R⁴ are each independently hydrogen, methyl, ethyl, propyl, butyl, pentyl, hexyl, heptyl, cyclobutyl, cyclopentyl, cyclohexyl, cycloheptyl or cyclooctyl. In one embodiment, R³ is independently methyl, and R⁴ is hydrogen.

Aspect 8. The compound of any one of the preceding aspects, wherein R^{1a}, R^{1b}, R^{2a} and R^{2b} are each independently hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN; wherein each said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl and cyclooctyl is optionally substituted with at least one substituent selected from hydrogen, F, Cl, Br, I, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl or —CN.

In one embodiment, R^{1a}, R^{1b}, R^{2a} and R^{2b} are each independently absent, hydrogen, halogen, or —C₁₋₆alkyl;
In one embodiment, R^{1a}, R^{1b}, R^{2a} and R^{2b} are each independently hydrogen, halogen, or —C₁₋₃alkyl;
In one embodiment, R^{1a}, R^{1b}, R^{2a} and R^{2b} are each independently hydrogen, or —C₁₋₃alkyl;
In one embodiment, R^{1a}, R^{1b}, R^{2a} and R^{2b} are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, or —CH₂CH₂OCH₂CH₃.
In one embodiment, R^{1a}, R^{1b}, R^{2a} and R^{2b} are each independently hydrogen, F, Cl, methyl, methoxy, cyclopropyl, —CF₃ or —CHF₂, or —CH₂OCH₃.
In one embodiment, R^{1a}, R^{1b}, R^{2a} and R^{2b} are each independently hydrogen.

Aspect 9. The compound of any one of the preceding aspects, wherein R⁵ and R⁶ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, or —CH₂CH₂OCH₂CH₃.

In one embodiment, R⁵ and R⁶ are each independently hydrogen, halogen, —C₁₋₆alkyl, —C₁₋₆alkoxy, —C₃₋₆ cycloalkyl or —CN; each said —C₁₋₆alkyl, —C₁₋₆alkoxy, or —C₃₋₆cycloalkyl is optionally substituted with at least one substituent selected from hydrogen, halogen, —C₁₋₈alkoxy, —C₃₋₈cycloalkyl or —CN.
In one embodiment, R⁵ and R⁶ are each independently hydrogen, halogen, —C₁₋₃alkyl, —C₁₋₃alkoxy, or —C₃₋₆cycloalkyl.
In one embodiment, R⁵ and R⁶ are each independently hydrogen, F, Cl, methyl, methoxy, cyclopropyl, —CF₃—CHF₂, or —CH₂OCH₃.

Aspect 10. The compound of any one of the preceding aspects, wherein R⁵ and R⁶ with the carbon atoms to which they are attached, form a 3-, 4-, 5-, 6-, 7- or 8-membered ring, said ring comprising 0-3 heteroatoms independently selected from nitrogen, oxygen and sulfur; wherein said ring is optionally substituted with at least one substituent F, Cl, Br, I, hydroxy, methyl, ethyl, propyl, butyl, pentyl, hexyl, heptyl or octyl.

In one embodiment, R⁵ and R⁶ with the carbon atoms to which they are attached, form a 3-, 4-, 5- or 6-membered ring, said ring is optionally substituted with at least one substituent selected from F, Cl, Br, I, hydroxy, methyl, ethyl, propyl, butyl, pentyl, hexyl, heptyl and octyl.

Aspect 11. The compound of any one of the preceding aspects, wherein R⁷ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CF₃, —CHF₂, —CN, —CH₂OCH₃, —CH₂OCH₂CH₃, —CH₂CH₂OCH₃, or —CH₂CH₂OCH₂CH₃;

In one embodiment, R⁷ is each independently absent, hydrogen, halogen, —C₁₋₆alkyl, —C₁₋₆alkoxy, —C₃₋₆ cycloalkyl or —CN; wherein each said —C₁₋₆alkyl, —C₁₋₆alkoxy or —C₃₋₆cycloalkyl is optionally substituted with at least one substituent selected from hydrogen, halogen, —C₁₋₆alkoxy, —C₃₋₆cycloalkyl and —CN.
In one embodiment, R⁷ is each independently absent, hydrogen, halogen, —C₁₋₃alkyl, or —C₁₋₃alkoxy.
In one embodiment, R⁷ are each independently hydrogen, F, Cl, methyl, methoxy, cyclopropyl, —CF₃ or —CHF₂, —CH₂OCH₃.
In one embodiment, R⁷ are each independently hydrogen.

Aspect 12. The compound of any one of the preceding aspects, wherein two R⁷ with the carbon atom(s) to which they are attached, form a 3-, 4-, 5-, 6-, 7- or 8-membered ring, said ring comprising 0-3 heteroatoms independently selected from nitrogen, oxygen and sulfur; said ring is optionally substituted with at least one substituent F, Cl, Br, I, hydroxy, methyl, ethyl, propyl, butyl, pentyl, hexyl, heptyl or octyl.

In one embodiment, two R⁷ with the carbon atom(s) to which they are attached, form a 3-, 4-, 5- or 6-membered ring, wherein said ring is optionally substituted with at least one substituent F, Cl, Br, I, hydroxy, methyl, ethyl, propyl, butyl, pentyl, hexyl, heptyl or octyl.

Aspect 13. The compound of any one of the preceding aspects, wherein R⁸ and R⁹ are each independently selected from hydrogen, F, Cl, Br, I, methyl, ethyl, propyl, butyl, pentyl, hexyl, cyclopropyl, cyclobutyl, cyclopentyl, cyclohexyl, cycloheptyl and cyclooctyl; wherein said methyl, ethyl, propyl, butyl, pentyl, hexyl, cyclopropyl, cyclobutyl, cyclopentyl, cyclohexyl, cycloheptyl or cyclooctyl is optionally substituted with at least one substituent selected from hydrogen, hydroxy, F, Cl, Br, I, methoxy, ethoxy, propoxy, butoxy, pentoxy and hexoxy.

In one embodiment, R⁸ and R⁹ are each independently selected from hydrogen, halogen, and —C₁-C₆alkyl; wherein said —C₁-C₆alky is optionally substituted with at least one substituent selected from hydrogen, halogen, hydroxy, and —C₁₋₆alkoxy.
In one embodiment, R⁸ and R⁹ are each independently selected from hydrogen, halogen, and —C₁-C₃alkyl; wherein said —C₁-C₃alky is optionally substituted with at least one substituent selected from hydrogen, halogen, and hydroxy.
In one embodiment, R⁸ and R⁹ are each independently hydrogen, F, Cl, Br, I, methyl, ethyl, propyl, butyl, pentyl, hexyl, heptyl, cyclobutyl, cyclopentyl, cyclohexyl, cycloheptyl, cyclooctyl, —CF₃, —CHF₂, —CH₂OH, or —CH₂CH₂OH.
In one embodiment, R⁸ is independently hydrogen, methyl, —CF₃, —CHF₂, or —CH₂OH, and R⁹ is F, methyl or —CH₂OH.

Aspect 14. The compound of any one of the preceding aspects, wherein R¹⁰ is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b}, —NR^{10a}SO₂R^{10b} and —CN; wherein each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl and phenyl is optionally substituted with at least one R^{10c};
R^{10a} and R^{10b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl and phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one substituent R^{10d};
R^{10c} and R^{10d} are each independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{10e}R^{10f}, —OR^{10e}, —SR^{10e}, —SO₂R^{10e}, —SO₂NR^{10e}R^{10f}, —C(O)R^{10e}, —CO₂R^{10e}, —C(O)NR^{10e}R^{10f}, —NR^{10e}COR^{10f}, —NR^{10e}CO₂R^{10f}, —NR^{10e}SO₂R^{10f} and —CN;
R^{10e} and R^{10f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl;
   In one embodiment, R¹⁰ is each independently selected from hydrogen, halogen, —C₁-C₆alkyl, —C₃-C₆cycloalkyl, —NR^{10a}R^{10b}, —OR^{10a}, —C(O)R^{10a}, —CO₂R^{10a}, —C(O)NR^{10a}R^{10b}, and —CN; wherein each of —C₁-C₆alkyl or —C₃-C₆cycloalkyl, is optionally substituted with at least one R^{10c};
R^{10a} and R^{10b} are each independently selected from hydrogen, —C₁-C₆alkyl, —C₃-C₆cycloalkyl, 3- to 8-membered heterocyclyl, C₆-C₁₂aryl, and 5- to 12-membered heteroaryl, wherein each of said —C₁-C₆alkyl, —C₃-C₆cycloalkyl, 3- to 8-membered heterocyclyl, C₆-C₁₂aryl, or 5- to 12-membered heteroaryl is optionally substituted with at least one substituent R^{10d};
R^{10c} and R^{10d} are each independently selected from halogen, hydrogen, —C₁-C₆alkyl, —C₁-C₆alkoxy, —C₃-C₆cycloalkyl, oxo (═O), —NR^{10e}R^{10f}, —OR^{10e}, —CO₂R^{10e}, —C(O)NR^{10e}R^{10f}, —NR^{10e}COR^{10f}, and —CN;
R^{10e} and R^{10f} are each independently selected from hydrogen, —C₁-C₃alkyl, —C₃-C₆cycloalkyl, 3- to 8-membered heterocyclyl, C₆-C₁₂aryl, and 5- to 12-membered heteroaryl.
   In one embodiment, R¹⁰ is each independently selected from hydrogen, halogen, —C₁-C₃alkyl, —C₃-C₆cycloalkyl, —NR^{10a}R^{10b}, —OR^{10a}, —C(O)R^{10a}, —CO₂R^{10a}, —C(O)NR^{10a}R^{10b}, and —CN; wherein each of —C₁-C₆alkyl or —C₃-C₆cycloalkyl, is optionally substituted with at least one halogen, hydrogen, oxo (═O), —NR^{10e}R^{10f} or —OR^{10e};
R^{10a} and R^{10b} are each independently selected from hydrogen, —C₁-C₃alkyl and —C₃-C₆cycloalkyl, wherein each of said —C₁-C₃alkyl or —C₃-C₆cycloalkyl is optionally substituted with at least one substituent halogen, hydrogen, oxo (═O), —NR^{10e}R^{10f} or —OR^{10e};
R^{10e} and R^{10f} are each independently selected from hydrogen or —C₁-C₃alkyl.
   In one embodiment, R¹⁰ is each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —NR^{10a}R^{10b}, —OR^{10a}, —SR^{10a}, —C(O)R^{10a}, —CO₂R^{10a}, —C(O)NR^{10a}R^{10b}, —NR^{10a}COR^{10b}, —NR^{10a}CO₂R^{10b}, —NR^{10a}SO₂R^{10b}, and —CN; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl or phenyl is optionally substituted with at least one R^{10c};
R^{10a} and R^{10b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl and phenyl;
R^{10c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{10e}R^{10f}, —OR^{10e} and —CN;
R^{10e} and R^{1f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl and 5- to 12-membered heteroaryl.
   In one embodiment, R¹⁰ is each independently selected from hydrogen, —F, —Cl, —Br, —I, methyl, ethyl, propyl, butyl, pentyl, hexyl, cyclopropyl, cyclobutyl, cyclopentyl, cyclohexyl, 3- to 8-membered heterocyclyl, —NR^{10a}R^{10b}, —OR^{10a}, —CO₂R^{10a}, and —C(O)NR^{10a}R^{10b}; each of methyl, ethyl, propyl, butyl, pentyl, hexyl, cyclopropyl, cyclobutyl, cyclopentyl, cyclohexyl or 3- to 8-membered heterocyclyl is optionally substituted with at least one R^{10c};
R^{10a} and R^{10b} are each independently selected from hydrogen, methyl, ethyl, propyl, butyl, pentyl, cyclopropyl, cyclobutyl, cyclopentyl, cyclohexyl, 3-membered heterocyclyl, 4-membered heterocyclyl, 5-membered heterocyclyl and 6-membered heterocyclyl;
R^{10c} is each independently selected from hydrogen, —F, —Cl, —Br, —I, methyl, ethyl, propyl, butyl, cyclopropyl, cyclobutyl, cyclopentyl, oxo (═O), —NR^{10e}R^{10f}, —OR^{10e} and —CN;
R^{10e} and R^{10f} are each independently selected from hydrogen, methyl, ethyl, propyl, cyclopropyl, cyclobutyl, and cyclopentyl.
   In one embodiment, R¹⁰ is each independently selected from H, F, Cl, Br, methyl, ethyl, propyl (n-propyl or iso-propyl), butyl(n-butyl, sec-butyl, iso-butyl or tert-butyl), cyclopropyl, cyclobutyl, cyclopentyl, cyclohexyl,

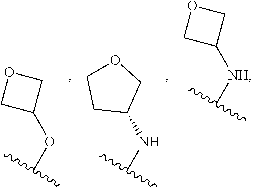

—COOH, —CONH₂, —CH₂OCH₃, and —CH₂OH.

Aspect 15. The compound of any one of the preceding aspects, wherein the

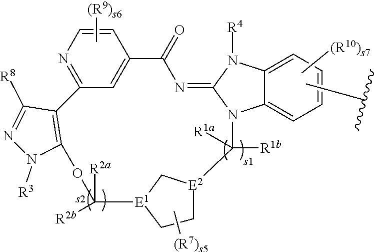

moiety is selected from

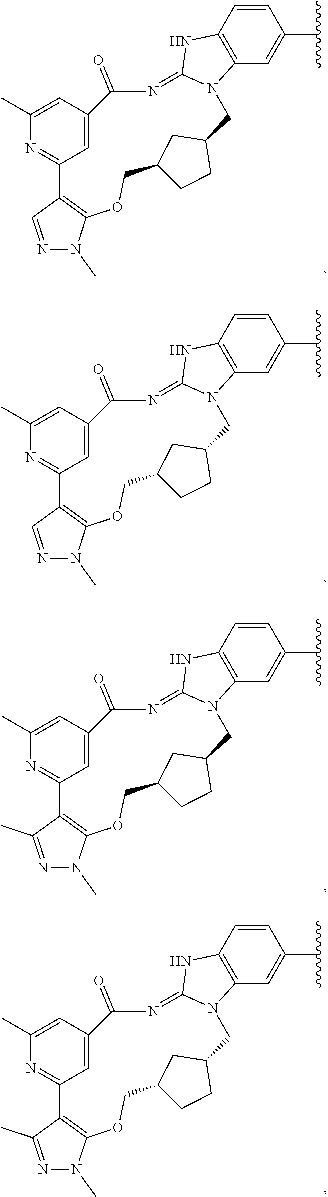 [SMILES: *c1ccc2c(c1)N1C[C@H]3CC[C@@H](COc4c(cnn4C)-c4cc(cc(C)n4)C(=O)/N=C/1N2)C3 ; *c1ccc2c(c1)N1C[C@@H]3CC[C@H](COc4c(cnn4C)-c4cc(cc(C)n4)C(=O)/N=C/1N2)C3 ; *c1ccc2c(c1)N1C[C@H]3CC[C@@H](COc4c(c(C)nn4C)-c4cc(cc(C)n4)C(=O)/N=C/1N2)C3 ; *c1ccc2c(c1)N1C[C@@H]3CC[C@H](COc4c(c(C)nn4C)-c4cc(cc(C)n4)C(=O)/N=C/1N2)C3]

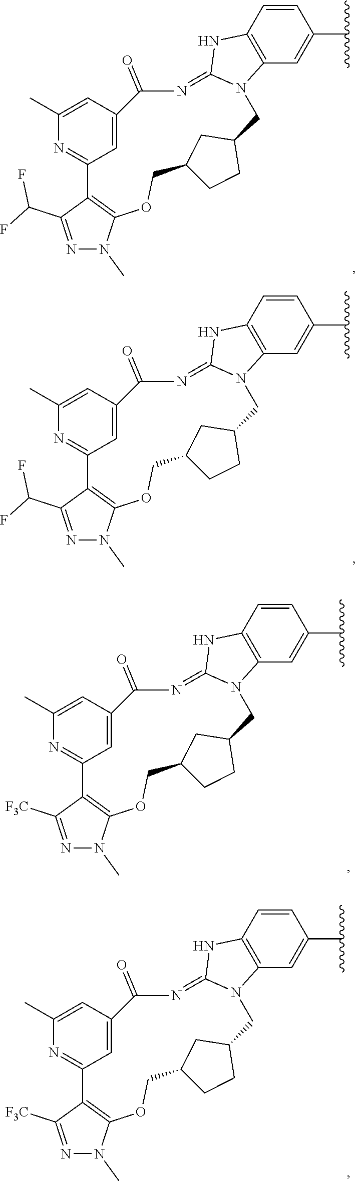 [SMILES: *c1ccc2c(c1)N1C[C@H]3CC[C@@H](COc4c(c(C(F)F)nn4C)-c4cc(cc(C)n4)C(=O)/N=C/1N2)C3 ; *c1ccc2c(c1)N1C[C@@H]3CC[C@H](COc4c(c(C(F)F)nn4C)-c4cc(cc(C)n4)C(=O)/N=C/1N2)C3 ; *c1ccc2c(c1)N1C[C@H]3CC[C@@H](COc4c(c(C(F)(F)F)nn4C)-c4cc(cc(C)n4)C(=O)/N=C/1N2)C3 ; *c1ccc2c(c1)N1C[C@@H]3CC[C@H](COc4c(c(C(F)(F)F)nn4C)-c4cc(cc(C)n4)C(=O)/N=C/1N2)C3]

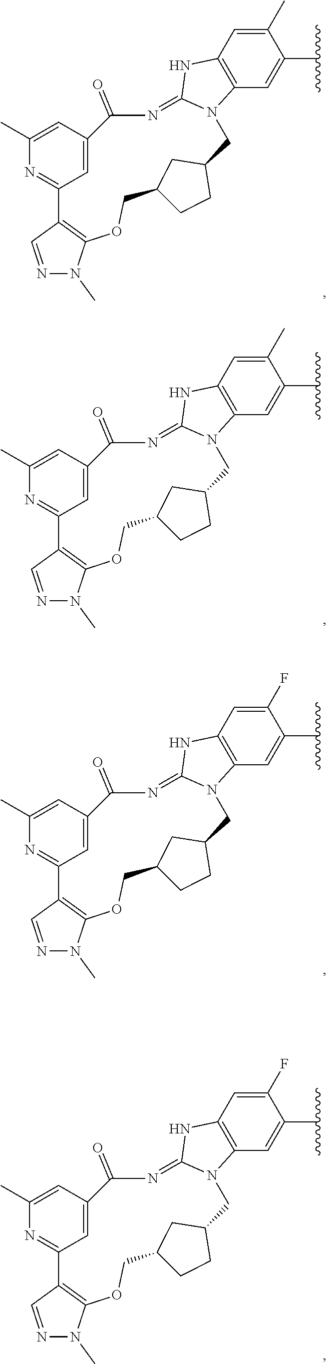 [SMILES: *c1cc2c(cc1C)N/C1=N\C(=O)c3cc(C)nc(c3)-c3cnn(C)c3OC[C@@H]3CC[C@@H](C3)CN12 ; *c1cc2c(cc1C)N/C1=N\C(=O)c3cc(C)nc(c3)-c3cnn(C)c3OC[C@H]3CC[C@H](C3)CN12 ; *c1cc2c(cc1F)N/C1=N\C(=O)c3cc(C)nc(c3)-c3cnn(C)c3OC[C@@H]3CC[C@@H](C3)CN12 ; *c1cc2c(cc1F)N/C1=N\C(=O)c3cc(C)nc(c3)-c3cnn(C)c3OC[C@H]3CC[C@H](C3)CN12]

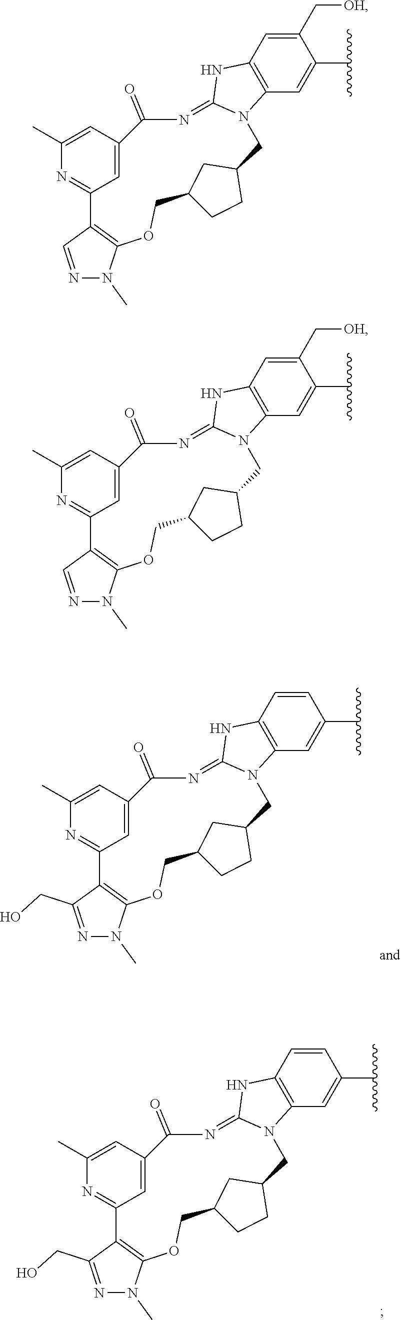 [SMILES: *c1cc2c(cc1CO)N/C1=N\C(=O)c3cc(C)nc(c3)-c3cnn(C)c3OC[C@@H]3CC[C@@H](C3)CN12 ; *c1cc2c(cc1CO)N/C1=N\C(=O)c3cc(C)nc(c3)-c3cnn(C)c3OC[C@H]3CC[C@H](C3)CN12 ; *c1ccc2c(c1)N1C[C@H]3CC[C@@H](COc4c(c(CO)nn4C)-c4cc(cc(C)n4)C(=O)/N=C/1N2)C3 ; *c1ccc2c(c1)N1C[C@H]3CC[C@@H](COc4c(c(CO)nn4C)-c4cc(cc(C)n4)C(=O)/N=C/1N2)C3]

In one embodiment, the

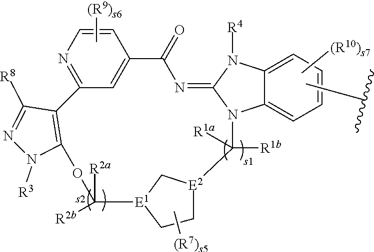

moiety is

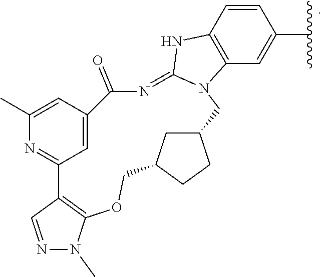 [SMILES: *c1ccc2c(c1)N1C[C@@H]3CC[C@H](COc4c(cnn4C)-c4cc(cc(C)n4)C(=O)/N=C/1N2)C3]

In one embodiment, the

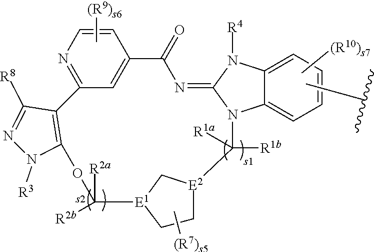

moiety is

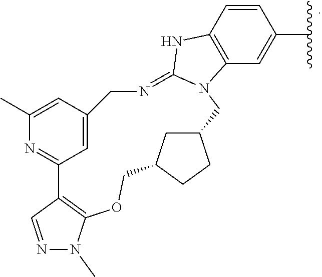 [SMILES: *c1ccc2c(c1)N1C[C@@H]3CC[C@H](COc4c(cnn4C)-c4cc(cc(C)n4)C/N=C/1N2)C3]

In one embodiment, the

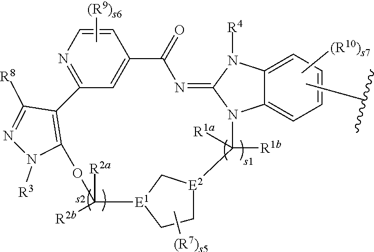

moiety is

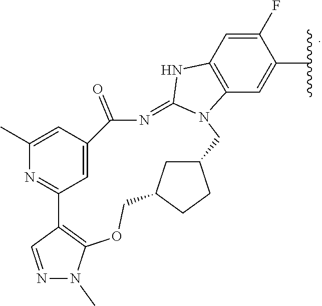 [SMILES: *c1cc2c(cc1F)N/C1=N\C(=O)c3cc(C)nc(c3)-c3cnn(C)c3OC[C@H]3CC[C@H](C3)CN12]

In one embodiment, the

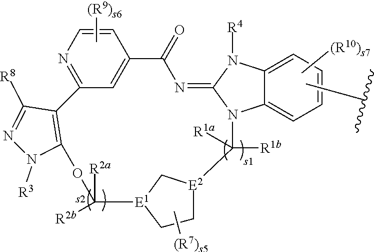

moiety is

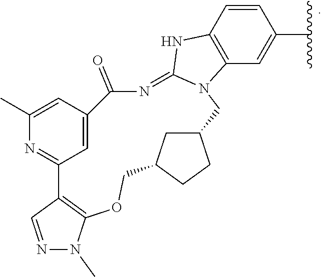 [SMILES: *c1ccc2c(c1)N1C[C@@H]3CC[C@H](COc4c(cnn4C)-c4cc(cc(C)n4)C(=O)/N=C/1N2)C3]

In another embodiment, the

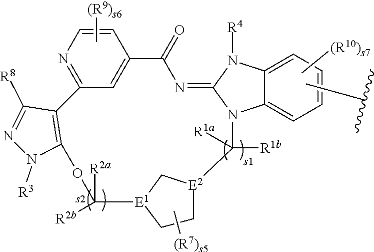

moiety is

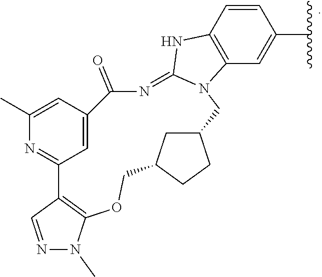 [SMILES: *c1ccc2c(c1)N1C[C@@H]3CC[C@H](COc4c(cnn4C)-c4cc(cc(C)n4)C(=O)/N=C/1N2)C3]

In another embodiment, the

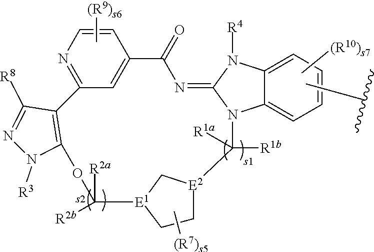

moiety is

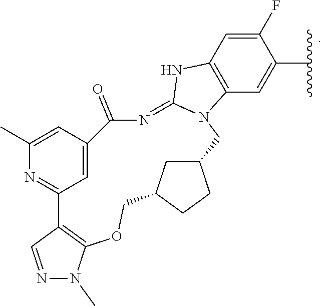 [SMILES: *c1cc2c(cc1F)N/C1=N\C(=O)c3cc(C)nc(c3)-c3cnn(C)c3OC[C@H]3CC[C@H](C3)CN12]

In another embodiment, the

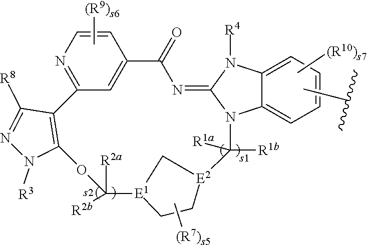

moiety is

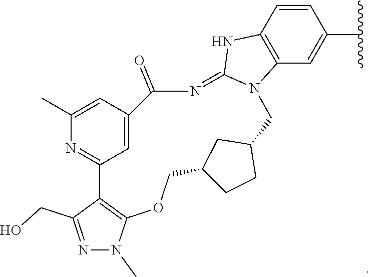 [SMILES: *c1ccc2c(c1)N1C[C@@H]3CC[C@H](COc4c(c(CO)nn4C)-c4cc(cc(C)n4)C(=O)/N=C/1N2)C3]

In another embodiment, the

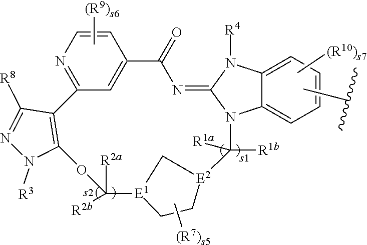

moiety is

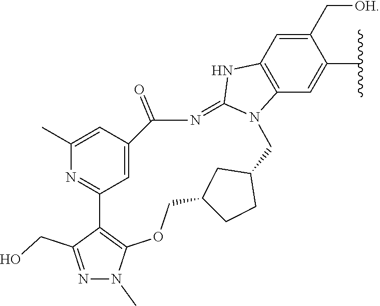 [SMILES: *c1cc2c(cc1CO)N/C1=N\C(=O)c3cc(C)nc(c3)-c3c(CO)nn(C)c3OC[C@H]3CC[C@H](C3)CN12]

In another embodiment, the

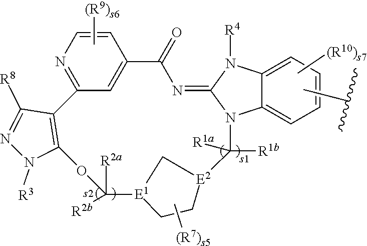

moiety is

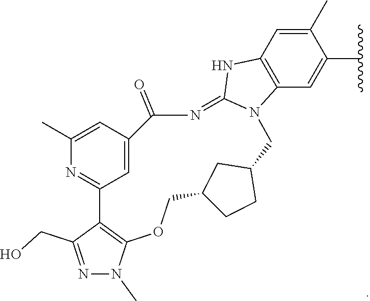 [SMILES: *c1cc2c(cc1C)N/C1=N\C(=O)c3cc(C)nc(c3)-c3c(CO)nn(C)c3OC[C@H]3CC[C@H](C3)CN12]

Aspect 16. The compound of any one of the preceding aspects, wherein R^{11a}, R^{11b}, R^{11c}, R^{11d}, R^{12a}, R^{12b}, R^{12c} and R^{12d} are each independently oxo, hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl; wherein each of said methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl is optionally substituted with at least one substituent selected from hydrogen, F, Cl, Br, I, methyl, ethyl, propyl, butyl, pentyl, hexyl, heptyl, octyl, —C₂₋₈alkenyl, —C₂₋₈alkynyl, methoxy, ethoxy, propoxy, butoxy, pentoxy, hexoxy, heptyloxy, octyloxy or —CN.

In one embodiment, R^{11a}, R^{11b}, R^{11c}, R^{11d}, R^{12a}, R^{12b}, R^{12c} and R^{12d} are each independently absent, oxo, hydrogen, halogen, —C₁₋₆alkyl, —C₁₋₆alkoxy or —C₃₋₆cycloalkyl; wherein each of said —C₁₋₆alkyl, —C₁₋₆alkoxy or —C₃₋₆cycloalkyl is optionally substituted with at least one substituent selected from hydrogen, halogen, —C₁₋₆alkoxy and —CN.
In one embodiment, R^{11a}, R^{11b}, R^{11c}, R^{11d}, R^{12a}, R^{12b}, R^{12c} and R^{12d} are each independently absent, oxo, hydrogen, halogen, —C₁₋₃alkyl or —C₁₋₃alkoxy.
In one embodiment, R^{11a}, R^{11b}, R^{11c}, R^{11d}, R^{12a}, R^{12b}, R^{12c} and R^{12d} are each independently oxo, hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl or cyclooctyl; preferably, R^{11a}, R^{11b}, R^{11c}, R^{11d}, R^{12a}, R^{12b}, R^{12c} and R^{12d} are each independently oxo, hydrogen, F, Cl, Br, I, methyl, ethyl or propyl.
In one embodiment, R^{11a}, R^{11b}, R^{11c}, R^{11d}, R^{12a}, R^{12b}, R^{12c} and R^{12d} are each independently hydrogen or methyl.

Aspect 17. The compound of any one of the preceding aspects, wherein L¹ is selected from —O—, —C(O)—, —N(R^{a})—, *^{L1}—C(O)N(R^{a})—**^{L1}, *^{L1}—C(O)O—**^{L1}, *^{L1}—N(R^{a})(O)—**^{L1}, *^{L1}—OC(O)—**^{L1},

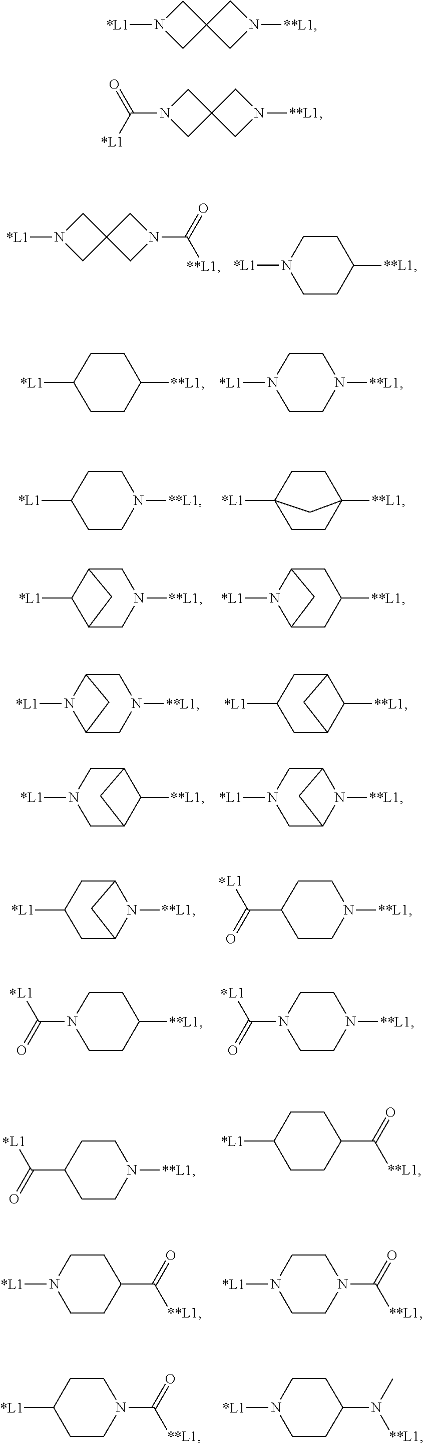

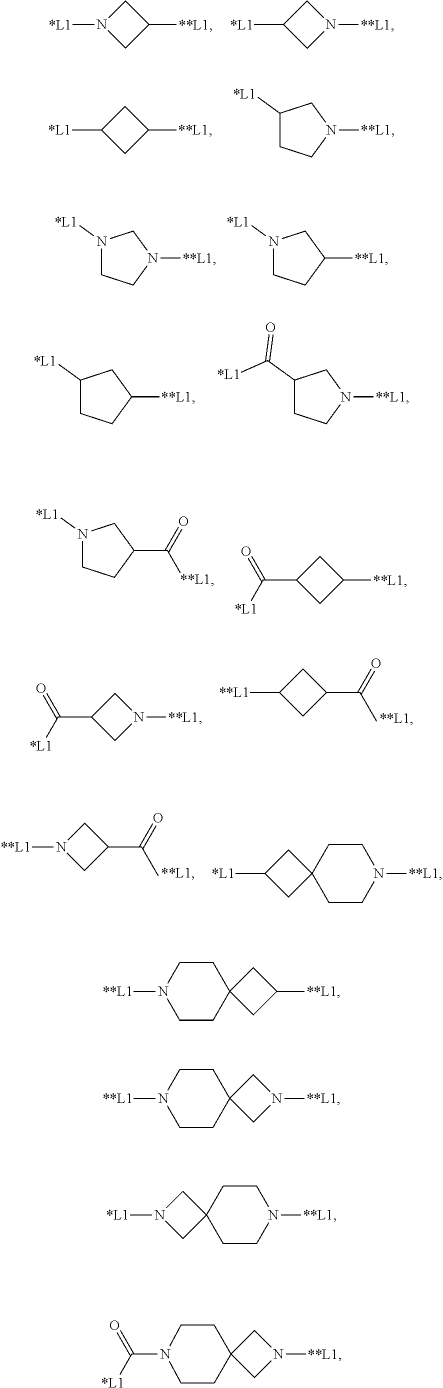

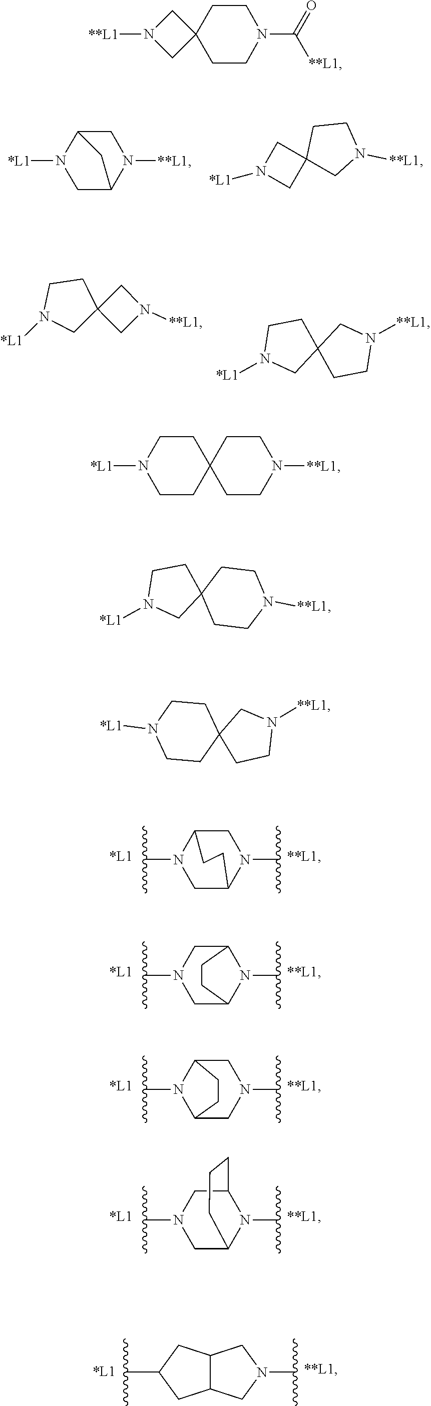

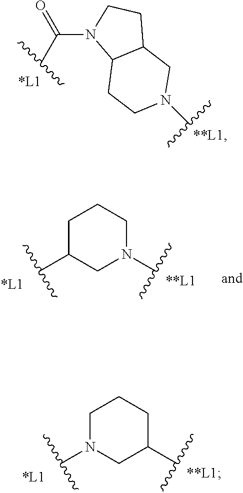 [SMILES: *C(=O)N1CCC2CN(*)CCC21 ; *C1CCCN(*)C1 ; *C1CCCN(*)C1]

wherein each of said

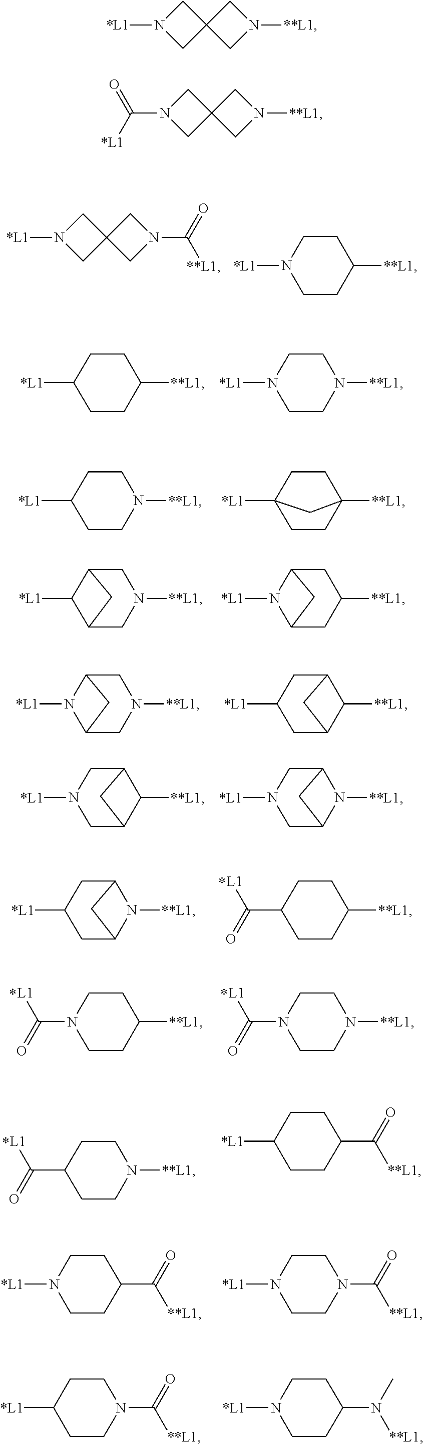

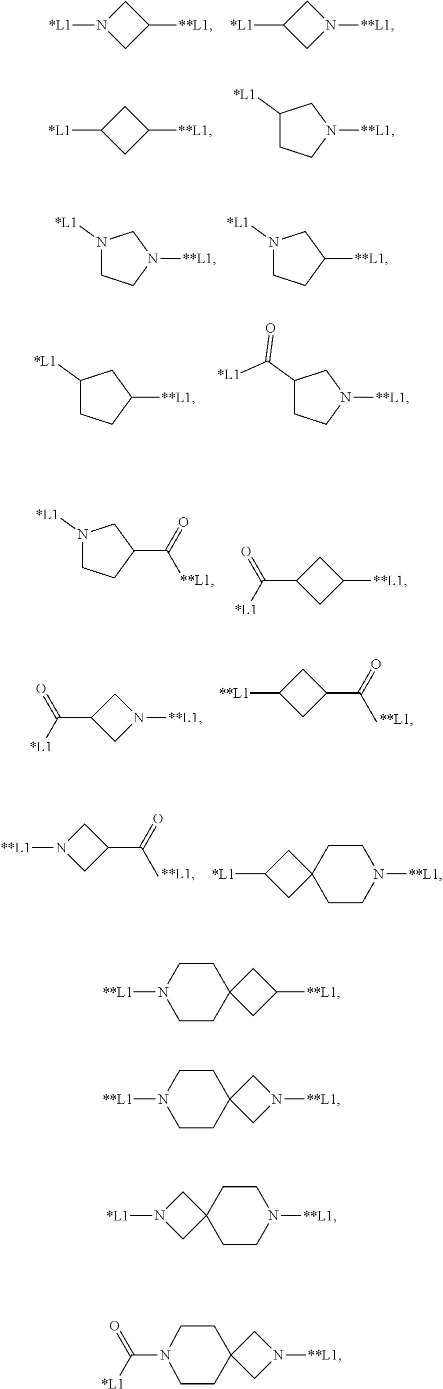

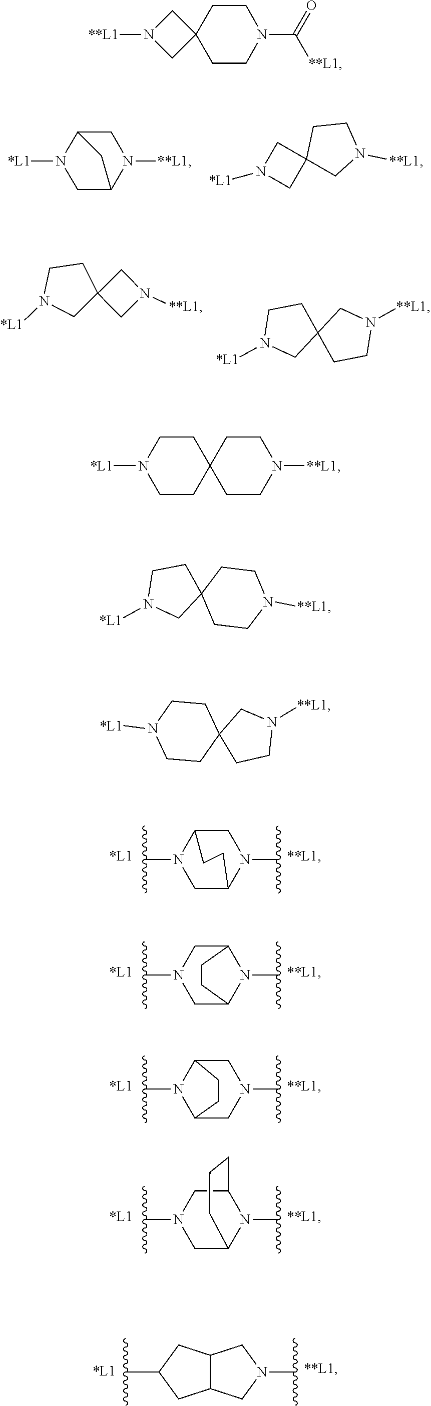

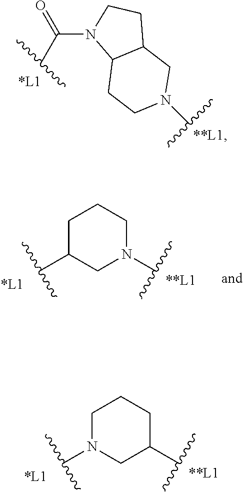 [SMILES: *C(=O)N1CCC2CN(*)CCC21 ; *C1CCCN(*)C1 ; *C1CCCN(*)C1]

is optionally substituted with at least one R^{L1c};
wherein each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen and sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, and cyclooctyl, wherein each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, or cyclooctyl is optionally substituted with at least one substituent selected from hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl.
In one embodiment, L¹ is selected from —O—, —C(O)—, —N(R^{a})—, *^{L1}—C(O)N(R^{a})—**^{L}, *^{L1}—C(O)O—**^{L1}, *^{L1}—N(R^{a})C(O)—**^{L1}, *^{L1}—OC(O)—**^{L1},

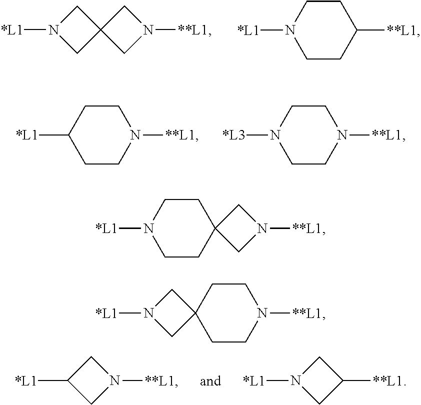

Aspect 18. The compound of any one of the preceding aspects, wherein L¹ is selected from —O—, —N(CH₃)—, —C(O)—, —NH—, *^{L1}—C(O)N(CH₃)—**^{L1}, *^{L1}—C(O)NH—**^{L1}, *^{L1}—C(O)O—**^{L1}, *^{L1}—C(O)N(C₂H₅)—**^{L1}, *^{L1}—C(O)N(C₃H₇)—**^{L1}, *^{L1}—N(CH₃)C(O)—**^{L1}, *^{L1}—NHC(O)—**^{L1}, *^{L1}—OC(O)—**^{L1}, *^{L1}—N(C₂H₅)C(O)—**^{L1}, *^{L1}—N(C₃H₇)C(O)—**^{L1},

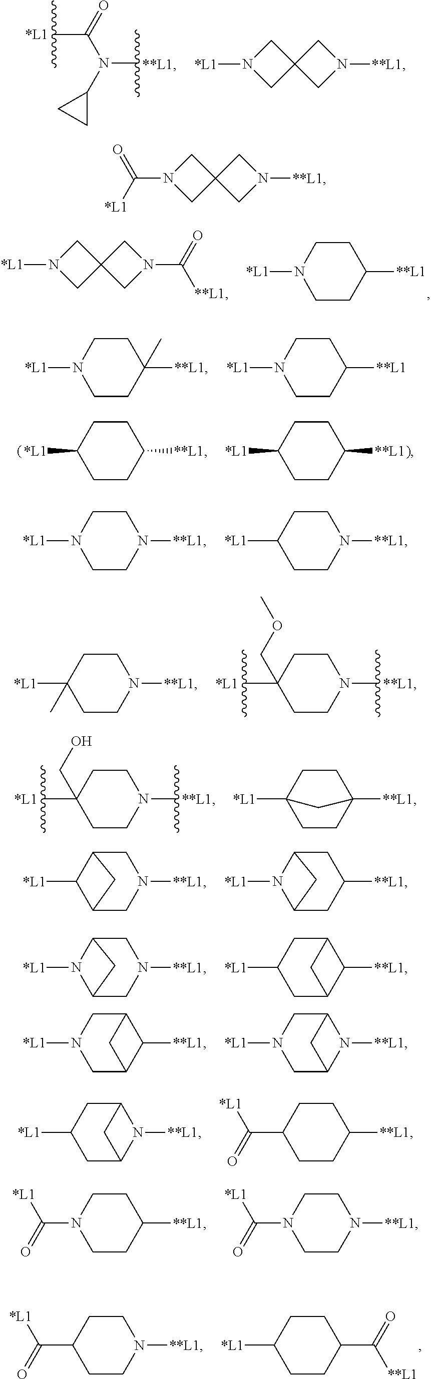

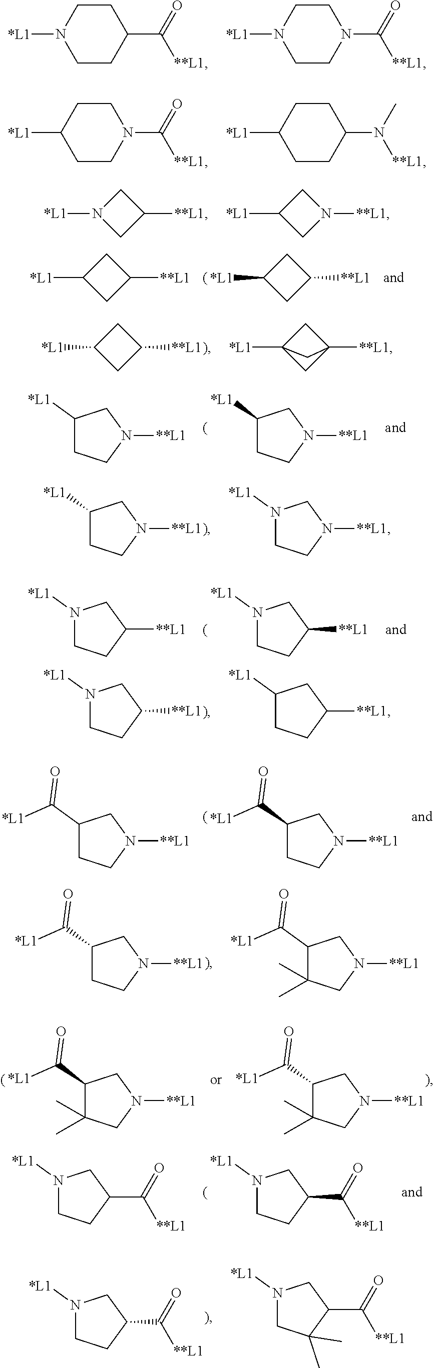

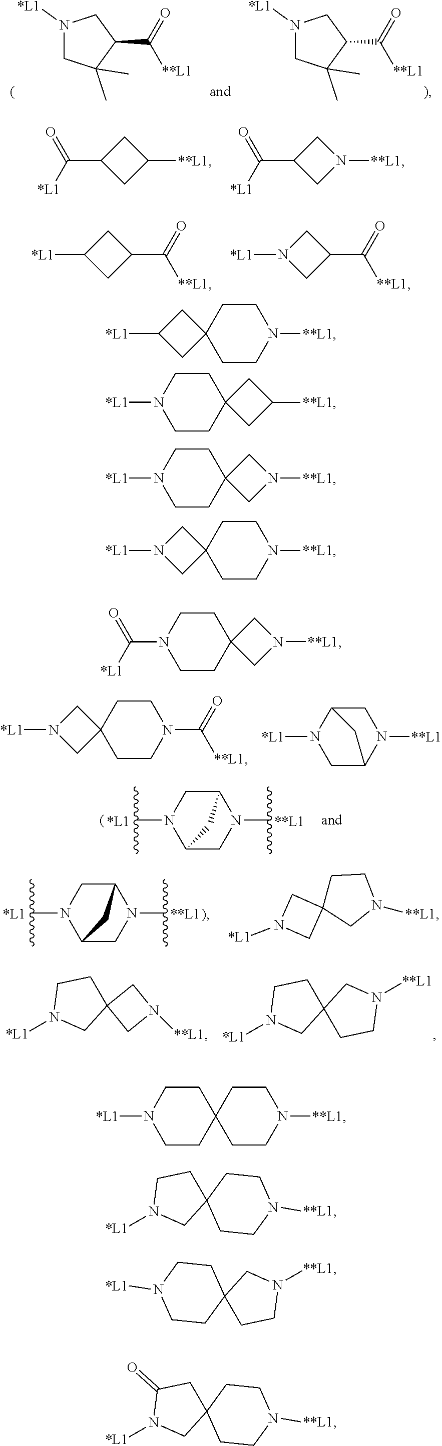

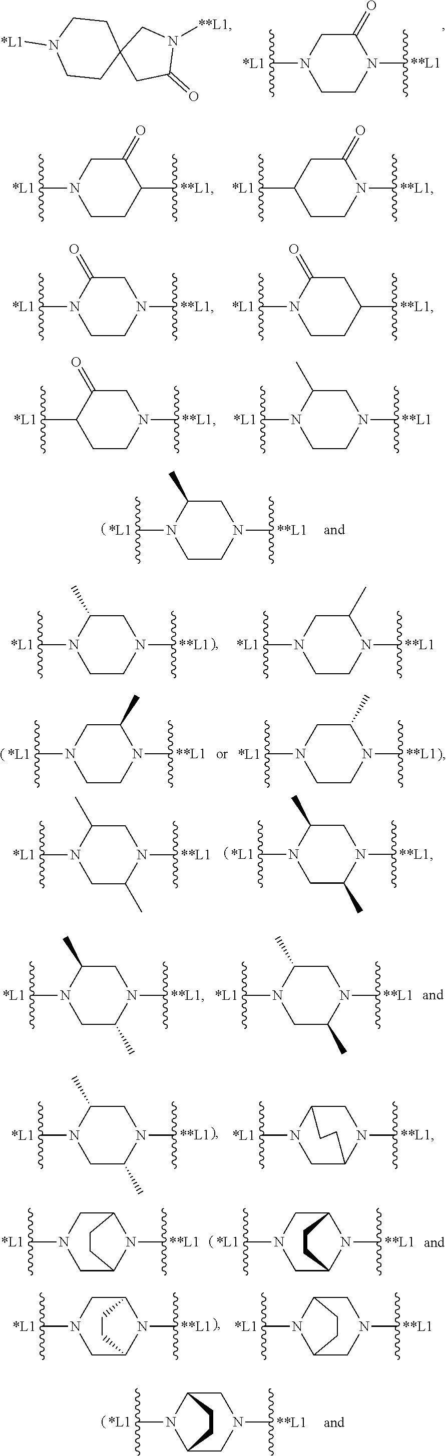

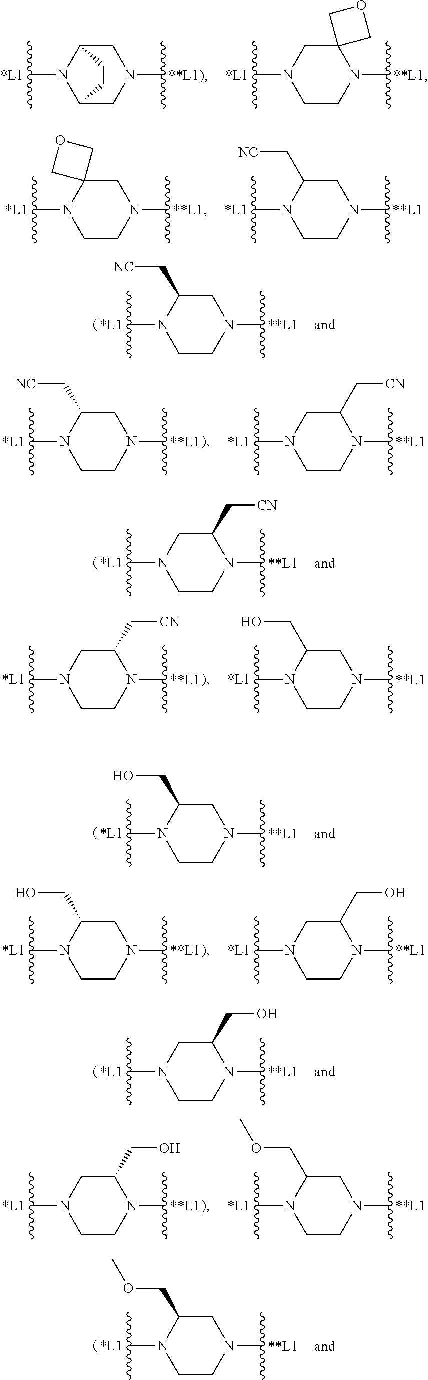

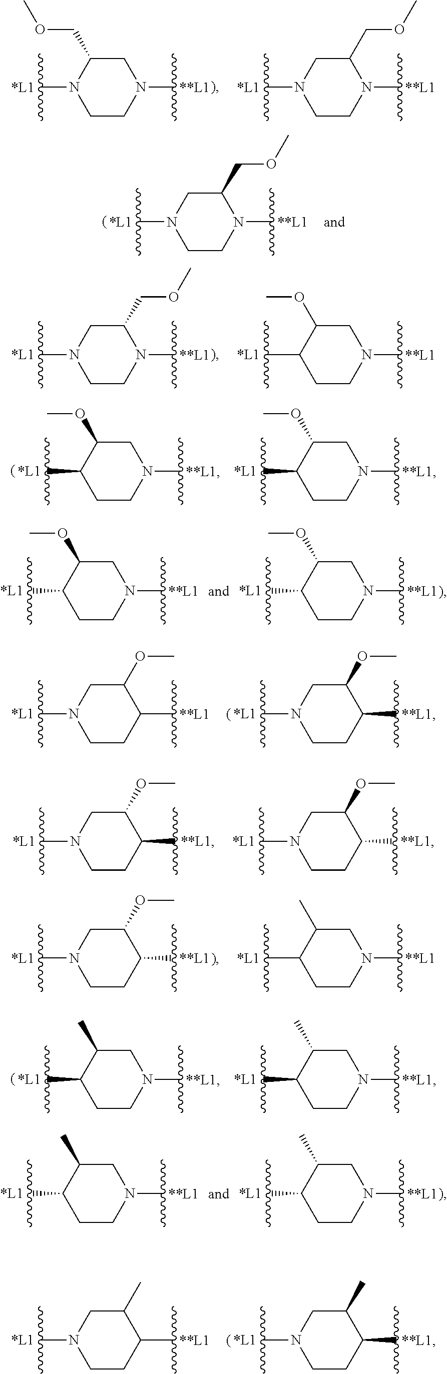

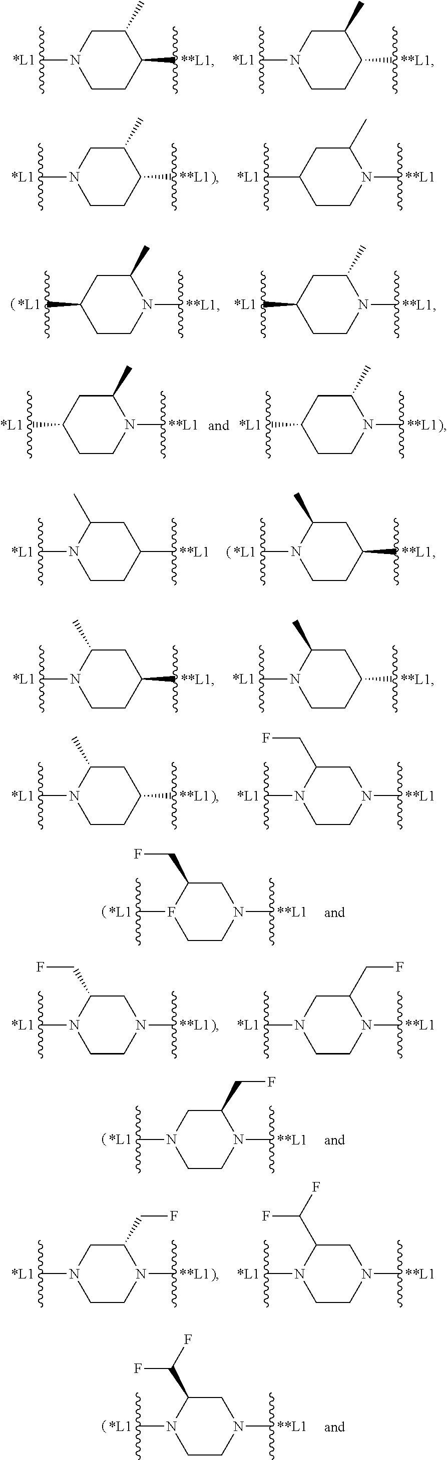

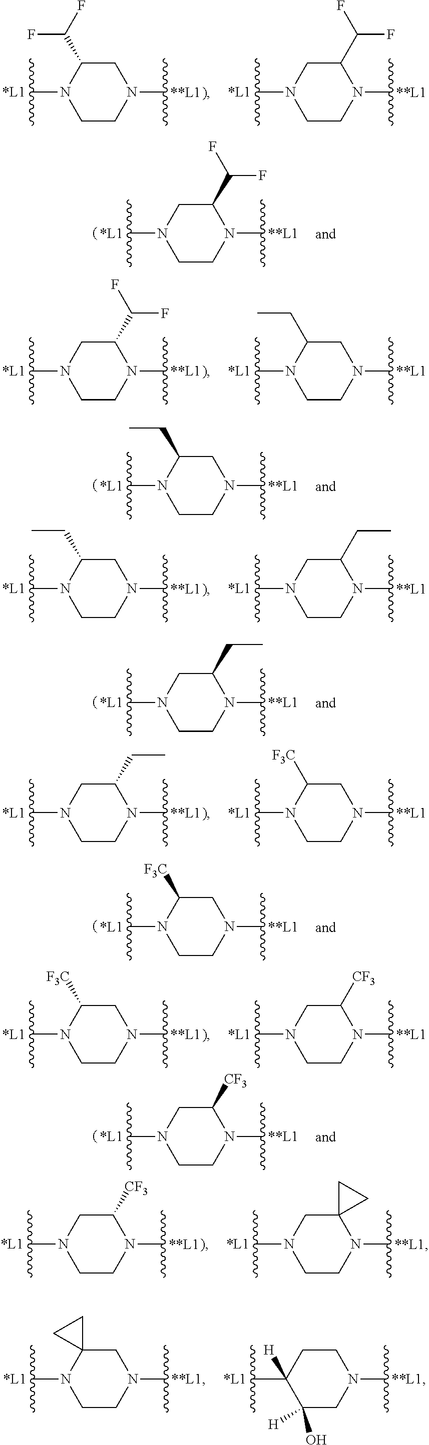

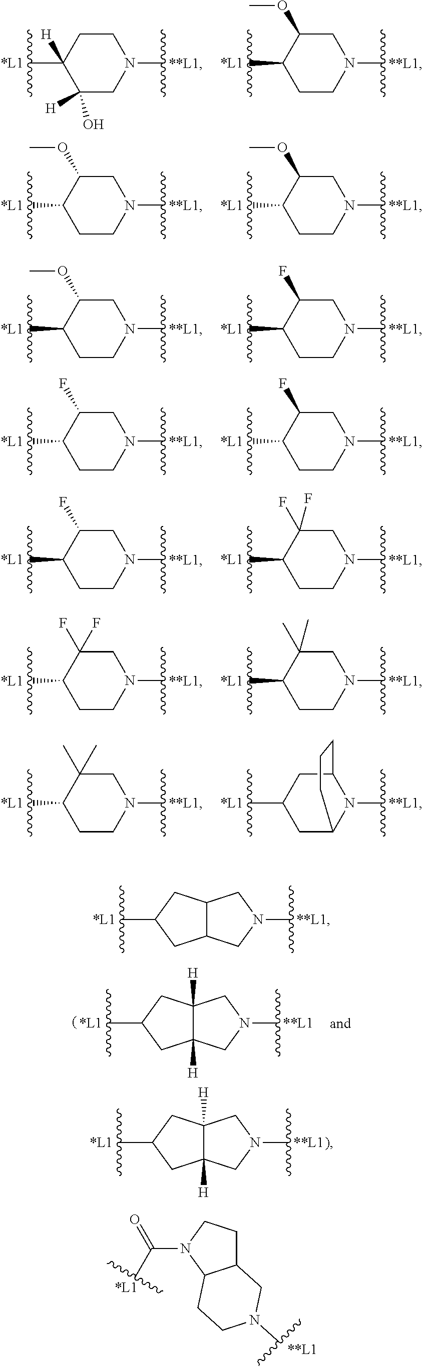

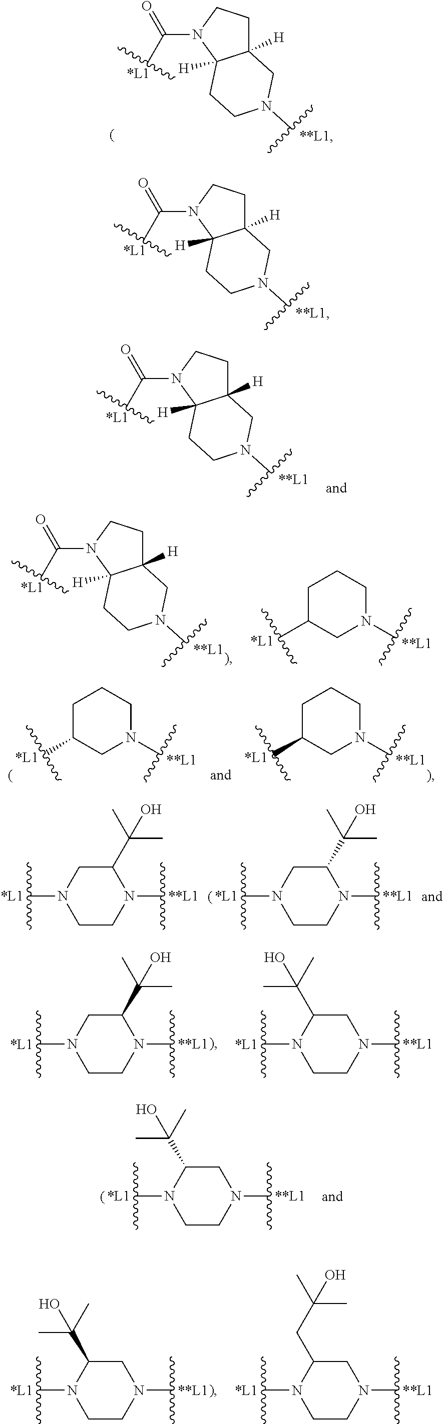

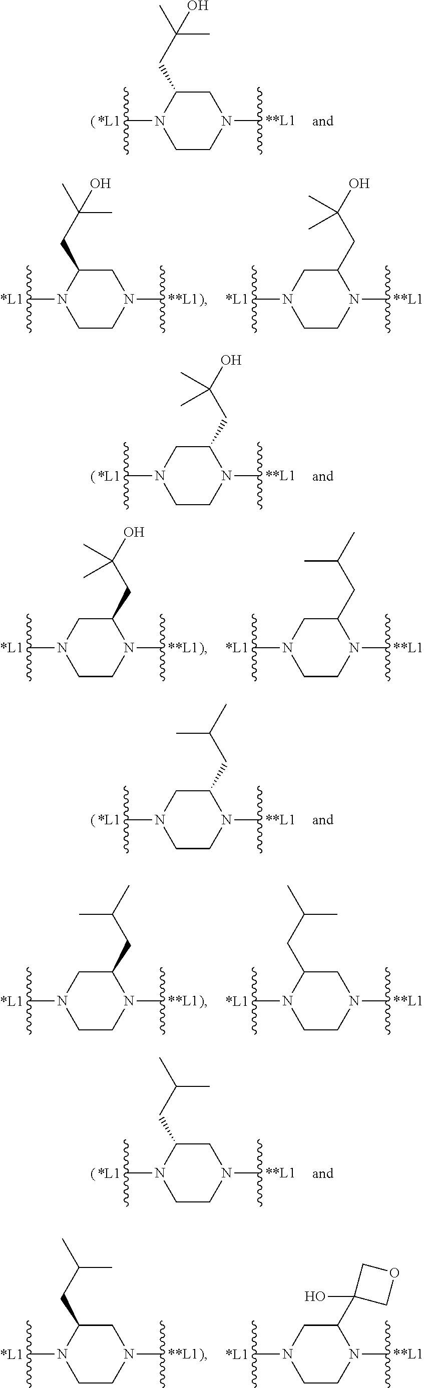

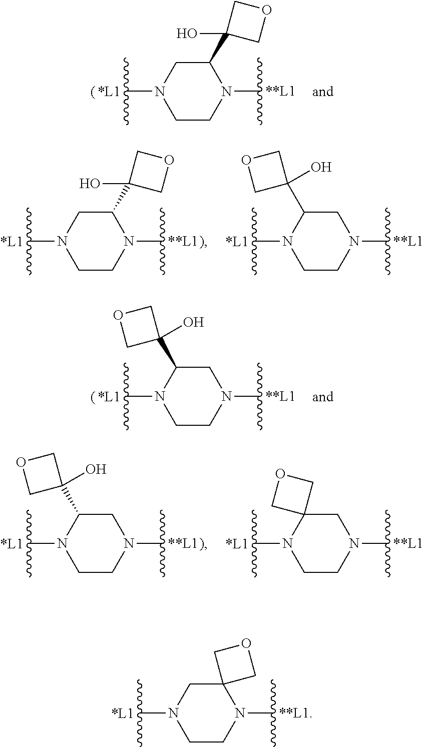

In one embodiment, L¹ is selected from

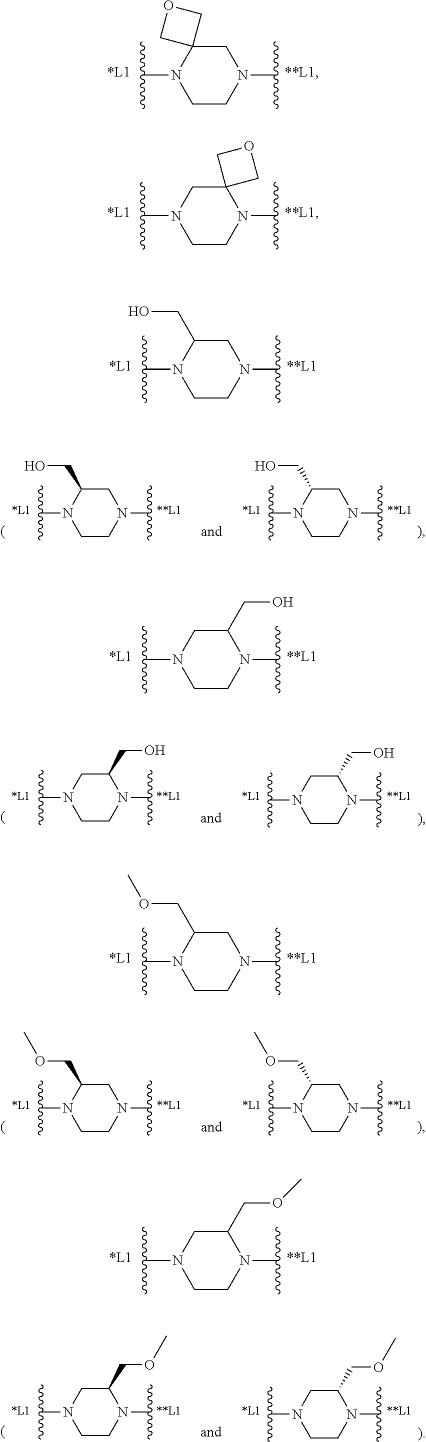

Aspect 19. The compound of any one of the preceding aspects, wherein L² is selected from —O—, —C(O)—, —N(R^{a})—, *^{L2}—C(O)N(R^{a})—**^{L2}, *^{L2}—C(O)O—**^{L2}, *^{L2}—N(R^{a})C(O)—**^{L2}, *^{L2}—OC(O)—**^{L2},

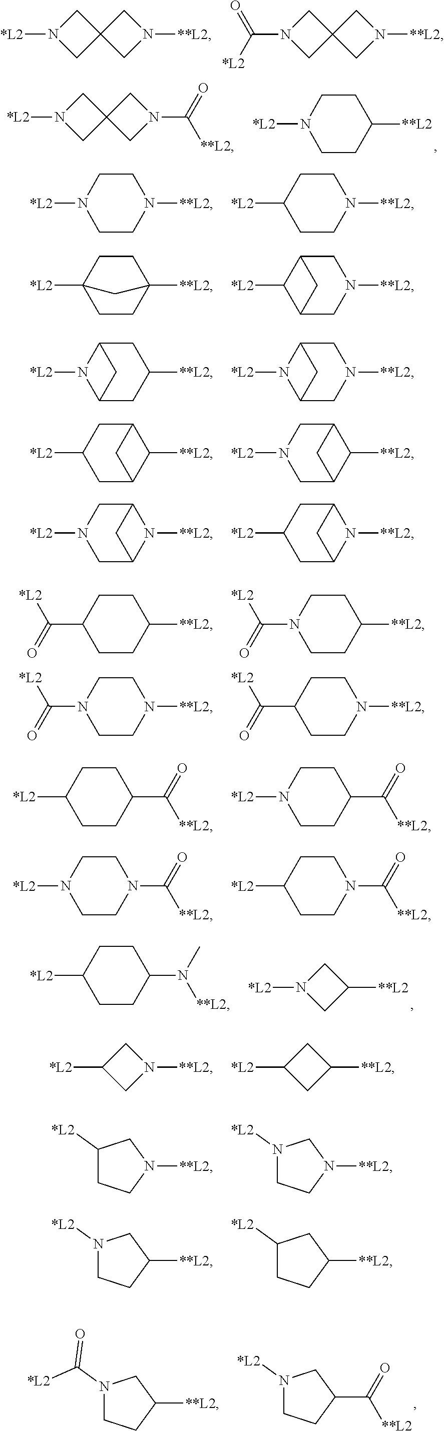

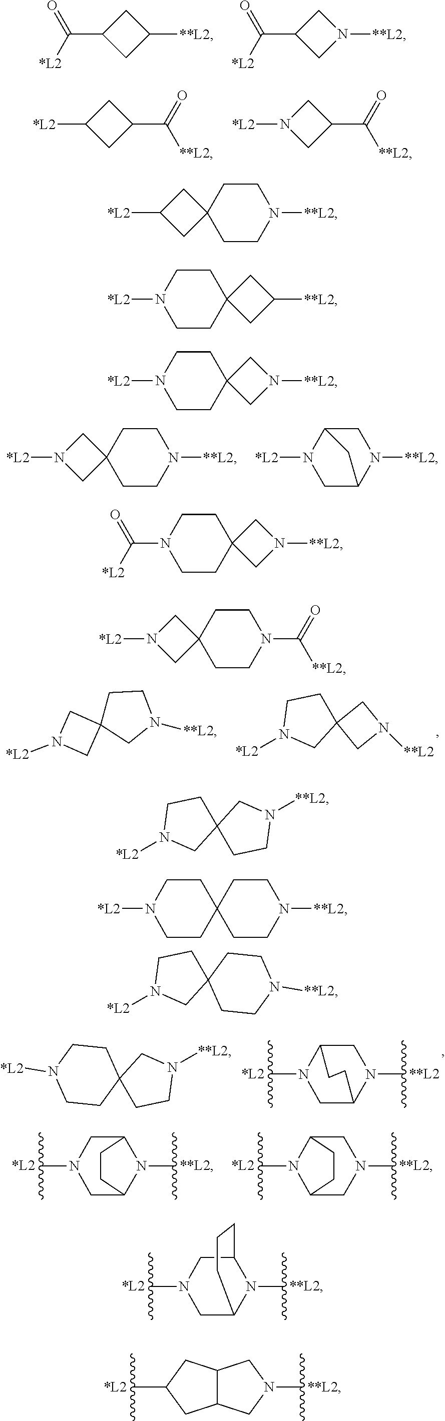

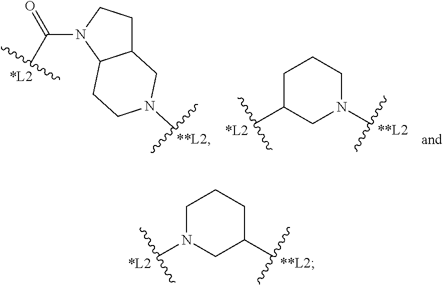 [SMILES: *C(=O)N1CCC2CN(*)CCC21 ; *C1CCCN(*)C1 ; *C1CCCN(*)C1]

wherein each of said

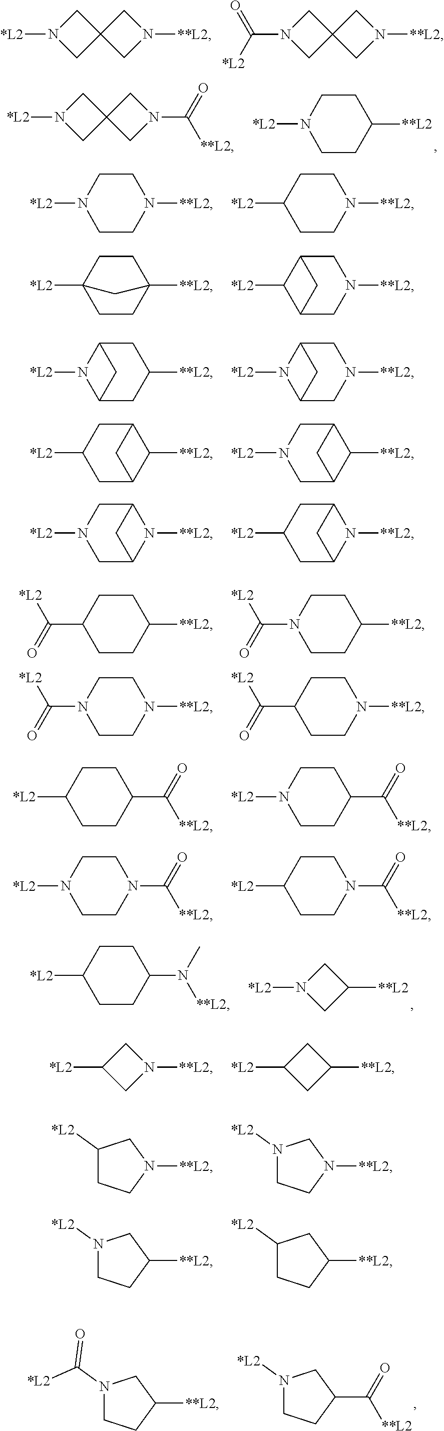

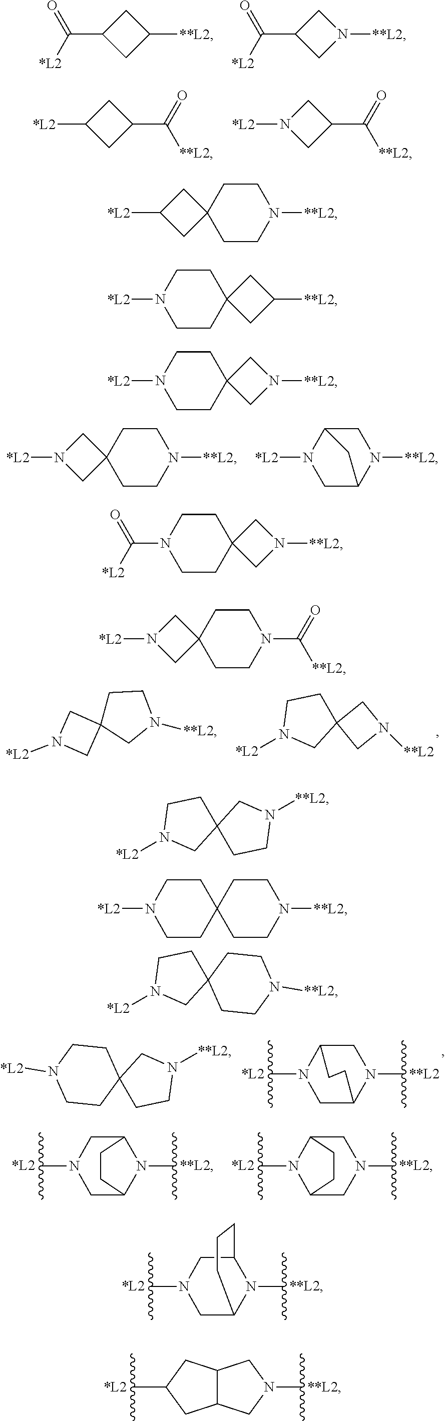

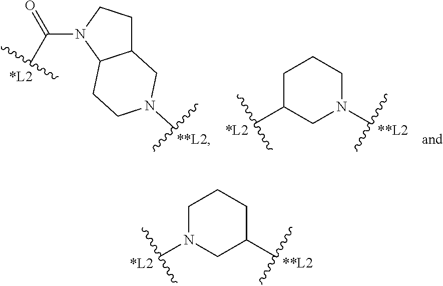 [SMILES: *C(=O)N1CCC2CN(*)CCC21 ; *C1CCCN(*)C1 ; *C1CCCN(*)C1]

is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen and sulfur; said ring is optionally substituted with at least one substituent F, Cl, Br, I, hydroxy, methyl, ethyl, propyl, butyl, pentyl, hexyl, heptyl, or octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, and cyclooctyl, wherein each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.
   In one embodiment, L² is selected from —O—, —C(O)—, —N(R^{a})—, *^{L2}—C(O)N(R^{a})—**^{L2}, *^{L2}—C(O)O—**^{L2}, *^{L2}—N(R^{a})C(O)—**^{L2}, *^{L2}—OC(O)—**^{L2},

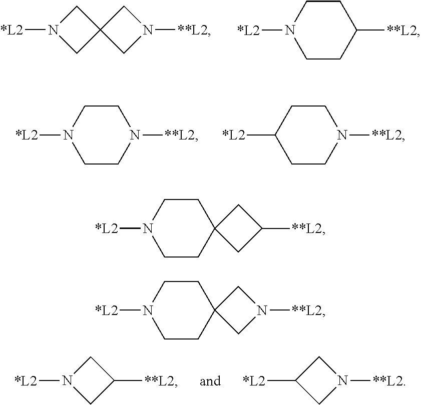

Aspect 20. The compound of any one of the preceding aspects, wherein L² is selected from —O—, —N(CH₃)—, —C(O)—, —NH—, *^{L2}—C(O)N(CH₃)—**^{L2}, *^{L2}—C(O)NH—**^{L2}, *^{L2}*C(O)O—*^{L2}, *^{L2}—C(O)N(C₂H₅)—**^{L2}, *^{L2}—C(O)N(C₃H₇)—**^{L2}, *^{L2}—N(CH₃)C(O)—**^{L2}, *^{L2}—NHC(O)—**^{L2}, *^{L2}*OC(O)—*^{L2}, *^{L2}—N(C₂H₅)C(O)—**^{L2}, *^{L2}—N(C₃H₇)C(O)—**^{L2},

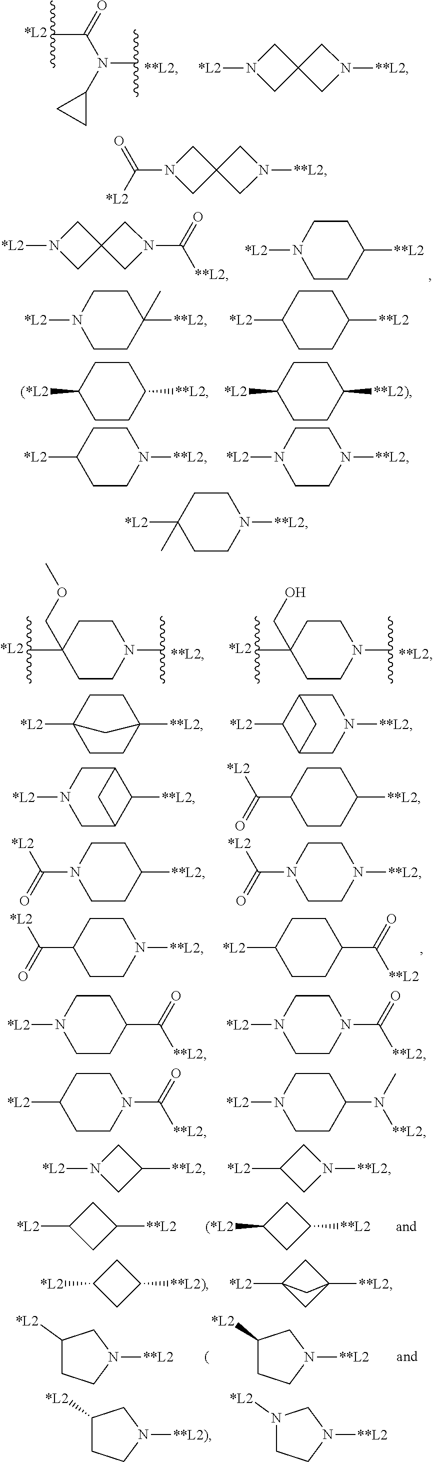

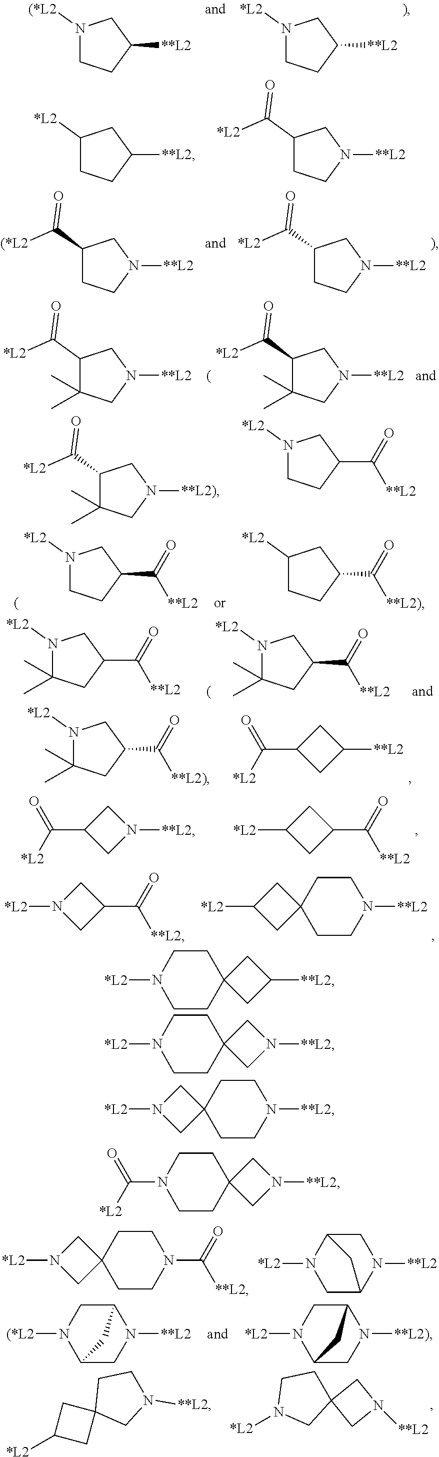

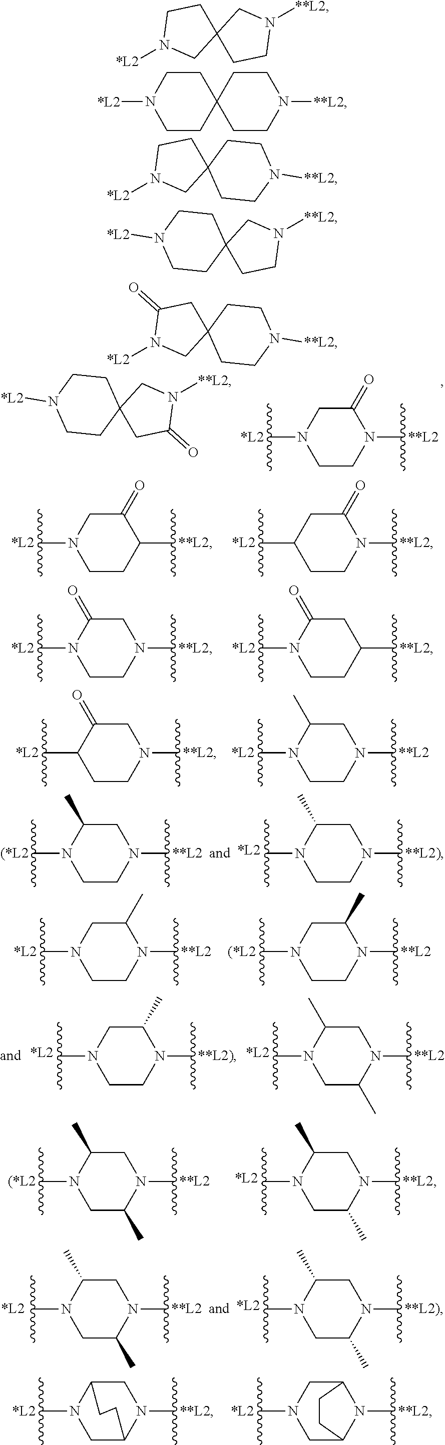

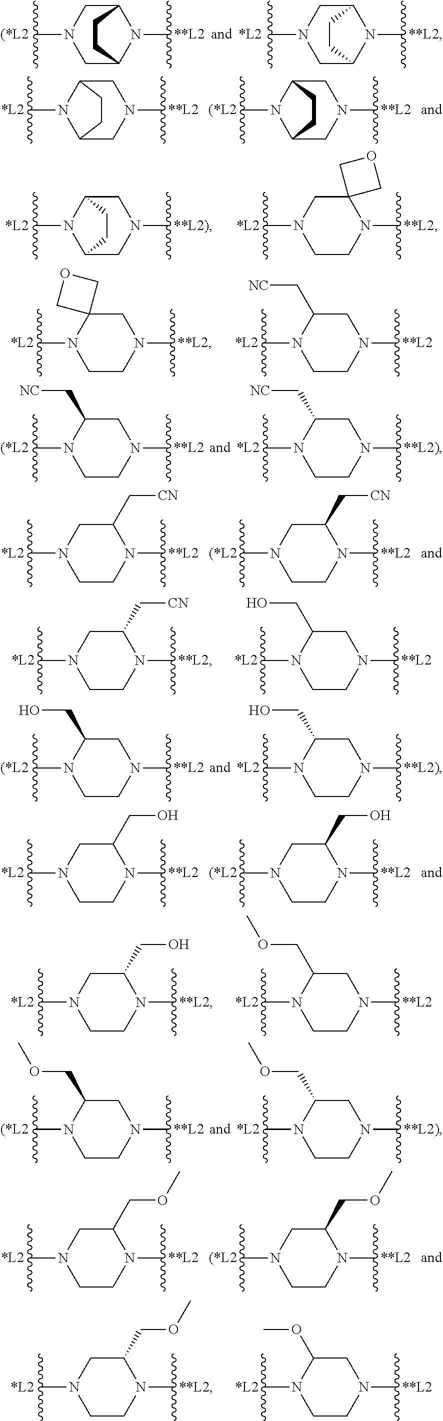

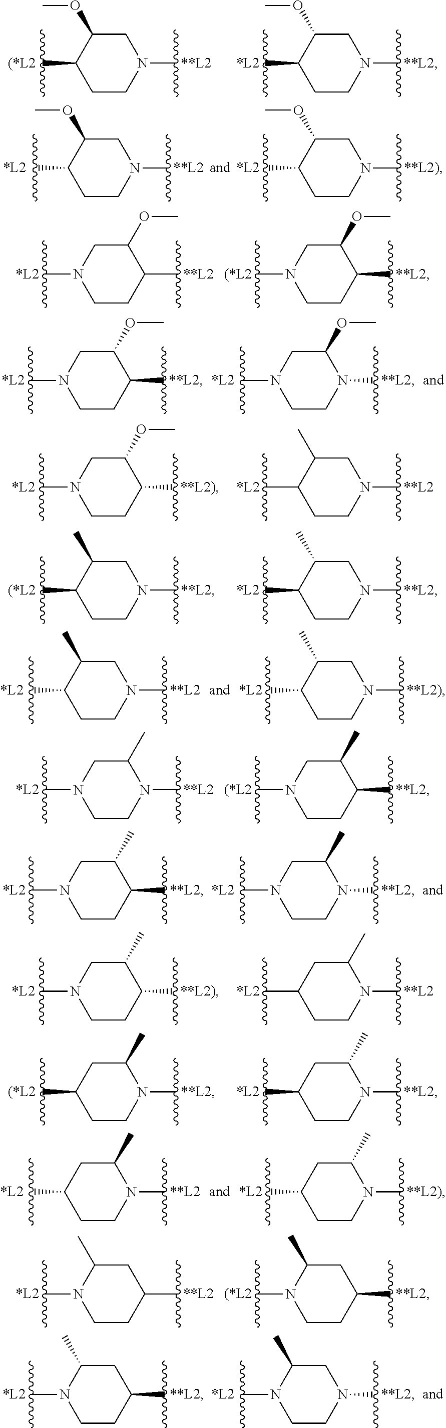

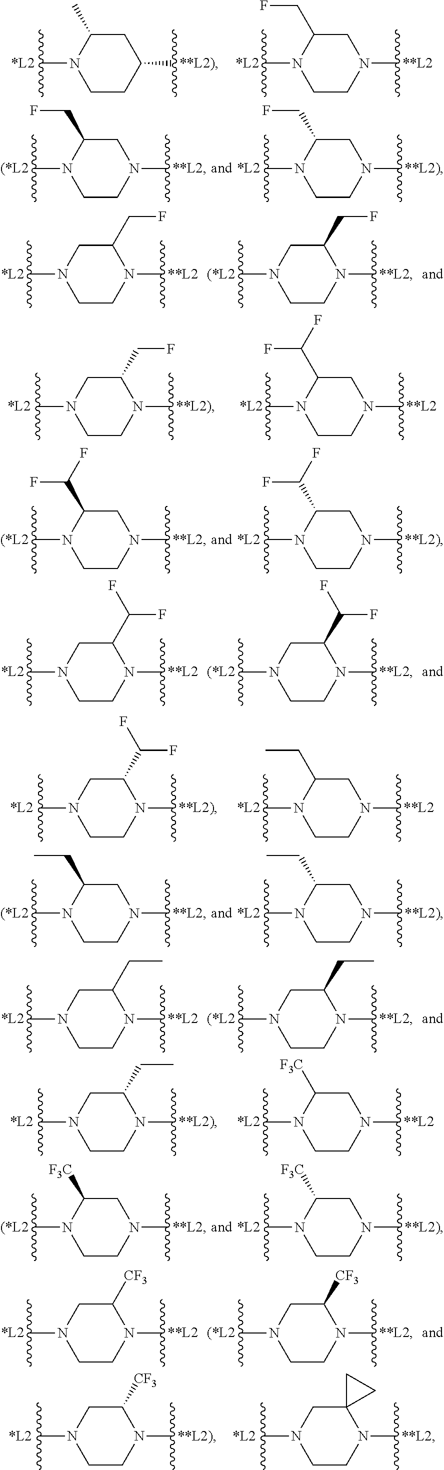

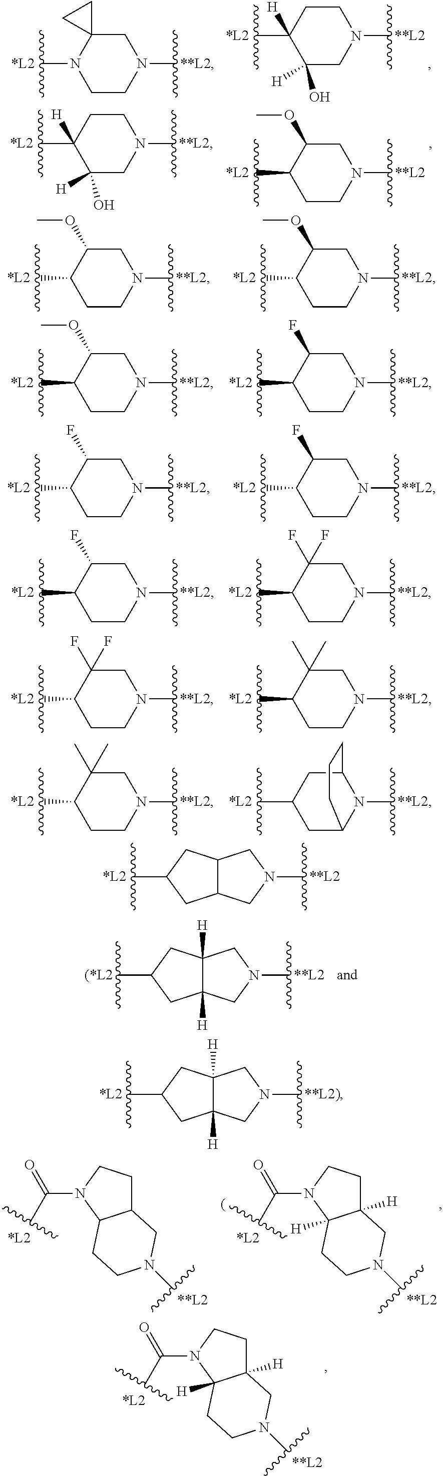

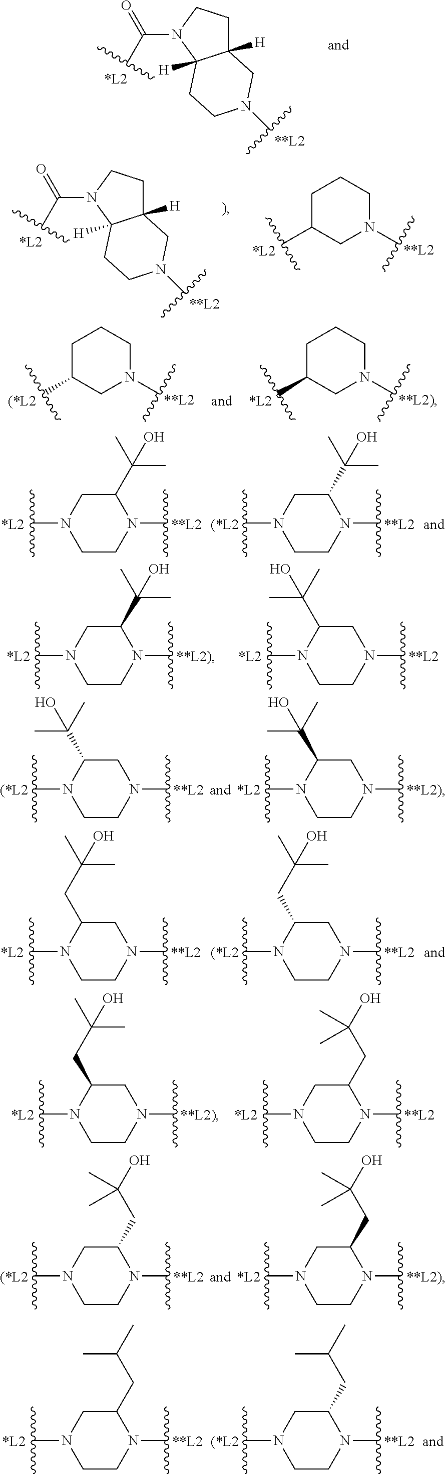

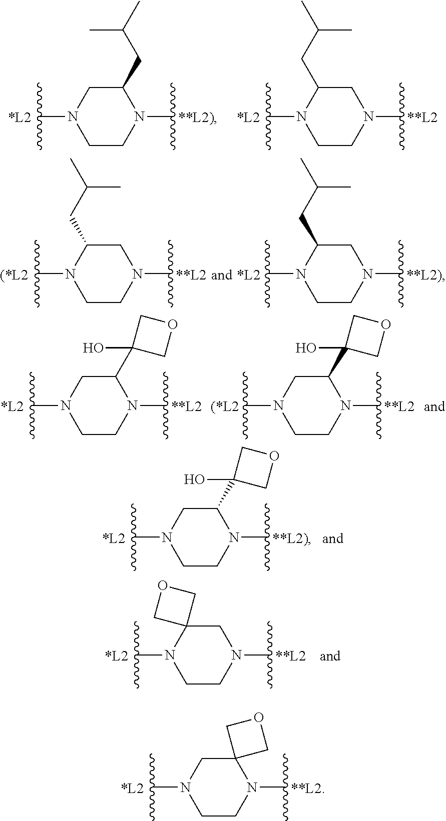

In one embodiment, L² is selected from

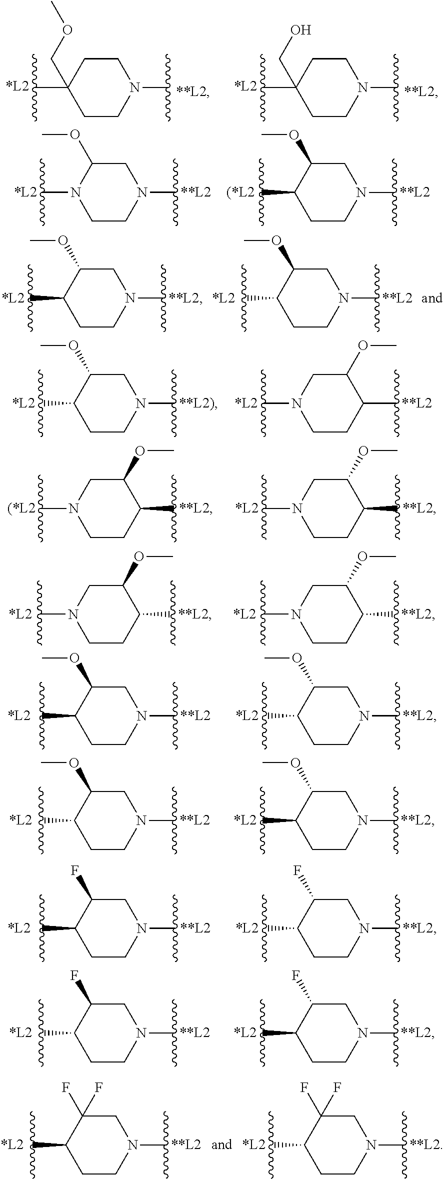

Aspect 21. The compound of any one of the preceding aspects, wherein L³ is selected from —O—, —N(R^{a})—, —C(O)—, *^{L3}—C(O)N(R^{a})—**^{L3}, *^{L3}—C(O)O—**^{L3}, *^{L3}—N(R^{a})C(O)—**^{L3}, *^{L3}—OC(O)—**^{L3},

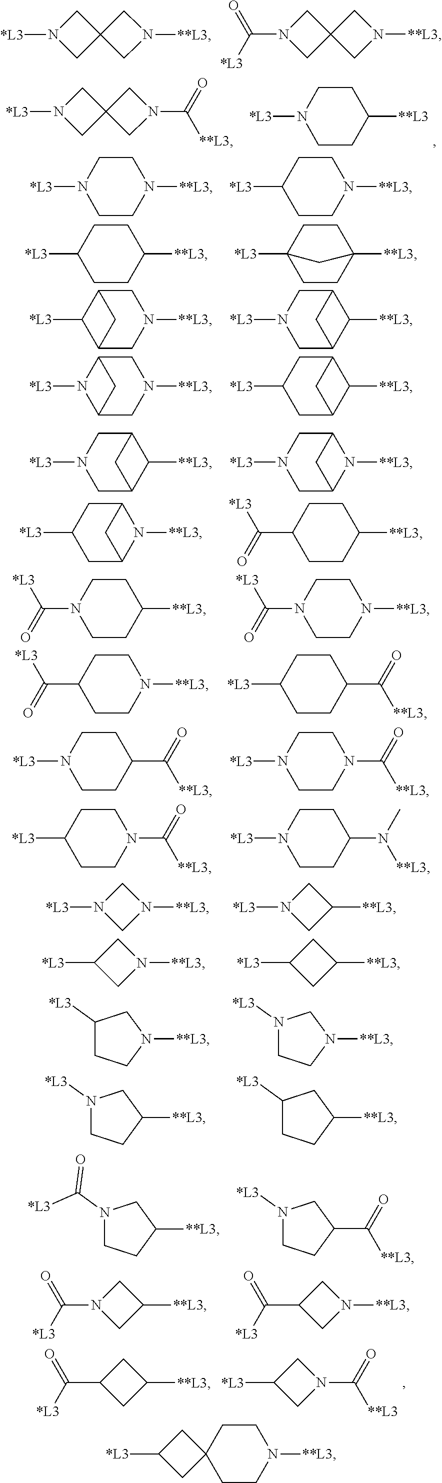

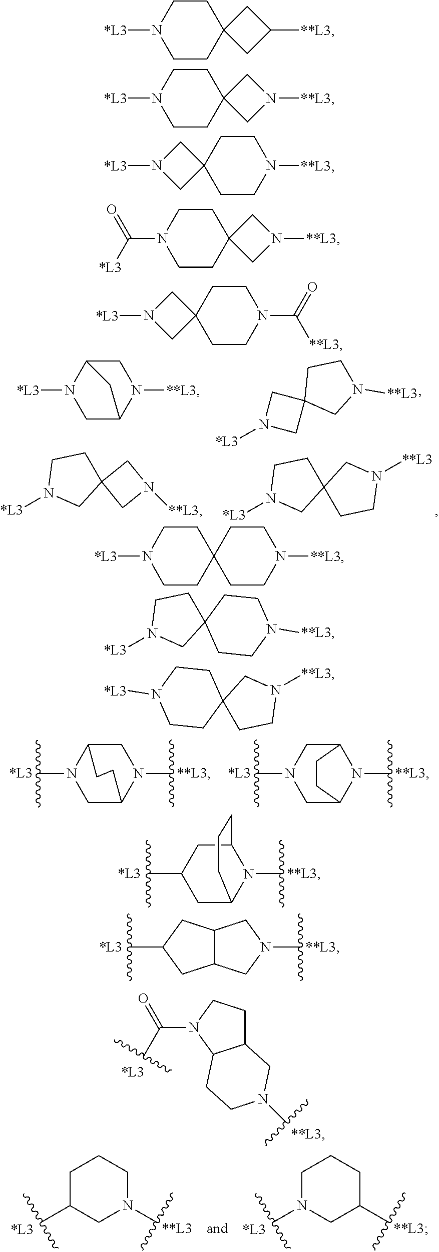

wherein each of said

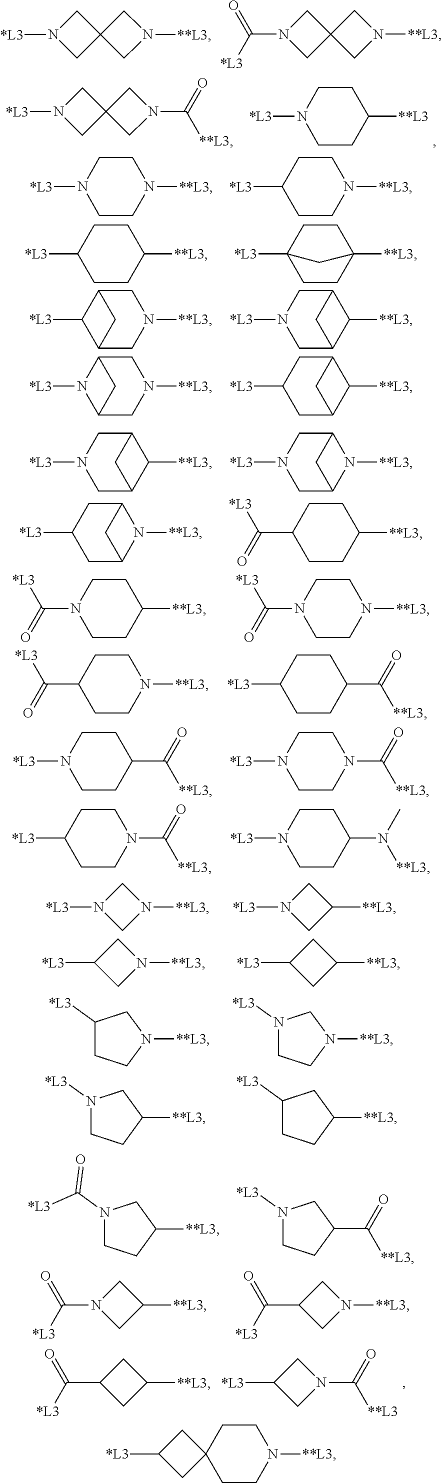

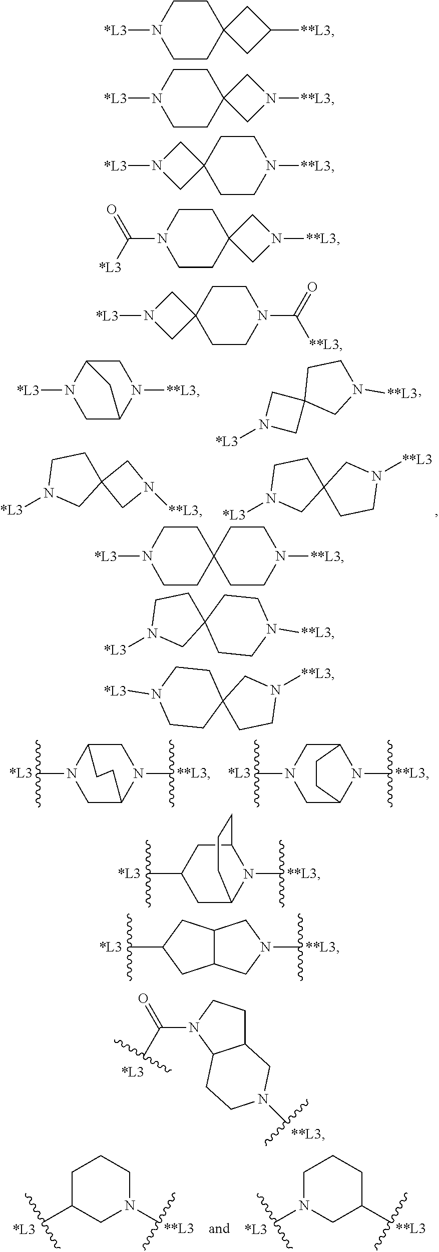

is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one R^{Lca},
R^{Lca} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen and sulfur; wherein said ring is optionally substituted with at least one substituent F, Cl, Br, I, hydroxy, methyl, ethyl, propyl, butyl, pentyl, hexyl, heptyl, octyl;
R^{a} is selected from hydrogen, methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, and cyclooctyl, wherein each of said methyl, ethyl, propyl, butyl, pentyl, hexyl, heptyl, octyl, methoxy, —C₂-C₈alkenyl, —C₂-C₈alkynyl, cyclopropyl, cyclobutyl, cyclopentyl, cyclohexyl, cycloheptyl, cyclooctyl is optionally substituted with at least one substituent selected from hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl.
   In one embodiment, L³ is selected from —O—, —C(O)—, —N(R^{a})—*^{L3}—C(O)N(R^{a})—**^{L3}, *^{L3}—C(O)—**^{L3}, *^{L3}—N(R^{a})C(O)—**^{L3}, *^{L3}—OC(O)—**^{L3},

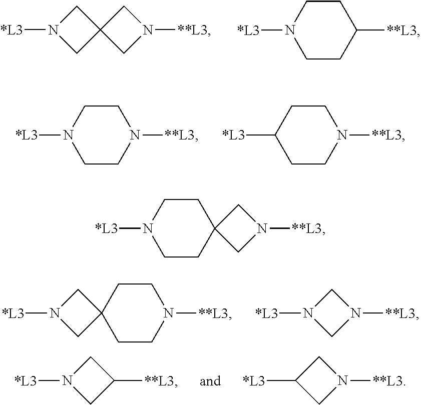

Aspect 22. The compound of any one of the preceding aspects, wherein L³ is selected from —O—, —N(CH₃)—, —C(O)—, —NH—, *^{L3}—C(O)N(CH₃)—**^{L3}, *^{L3}—C(O)NH—**^{L3}, *^{L3}—C(O)O**^{L3}, *^{L3}—C(O)N(C₂H₅)—**^{L3}, *^{L3}—C(O)N(C₃H₇)—**^{L3}, *^{L3}—N(CH₃)C(O)—**^{L3}, *^{L3}—NHC(O)—**^{L3}, *^{L3}—OC(O)—**^{L3}, *^{L3}—N(C₂H₅)C(O)—**^{L3}, *^{L3}—N(C₃H₇)C(O)—**^{L3},

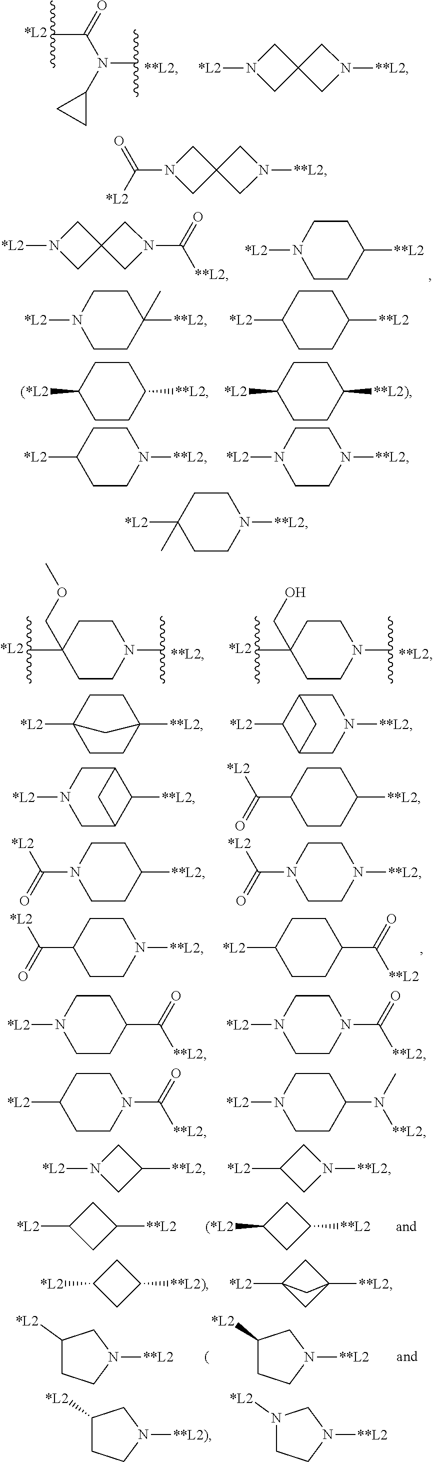

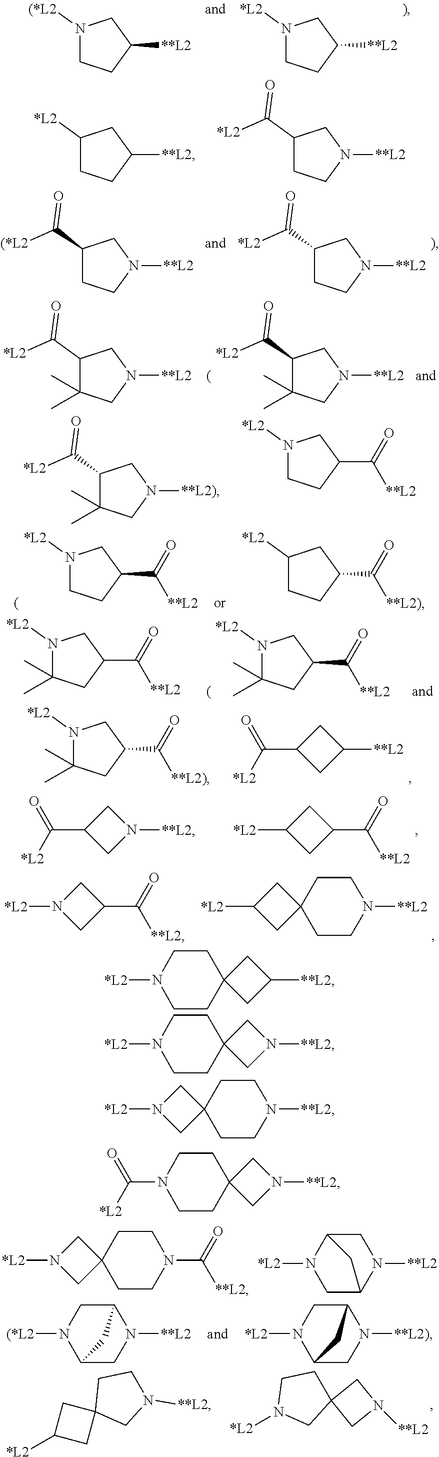

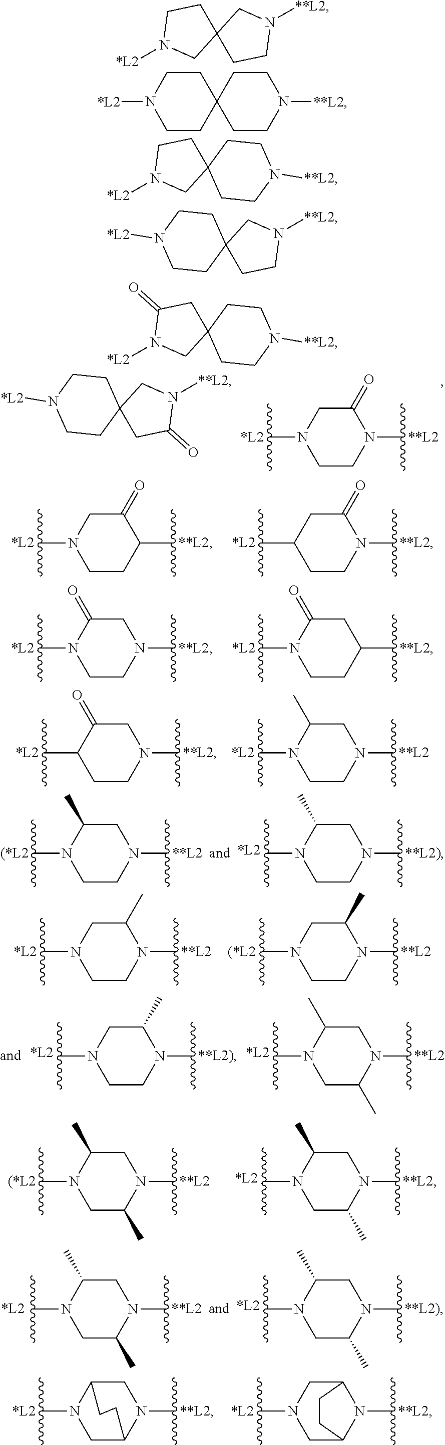

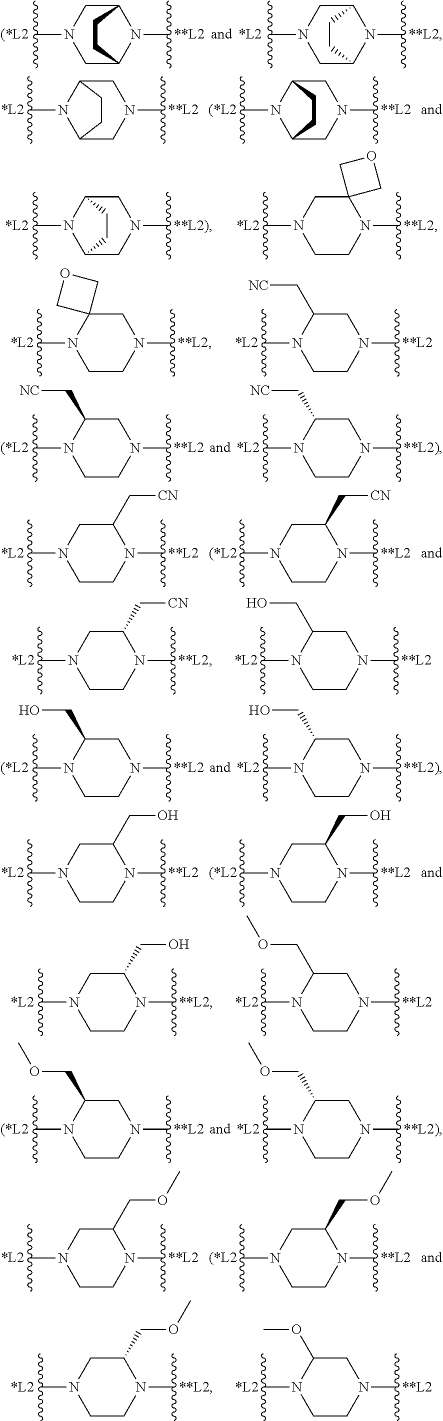

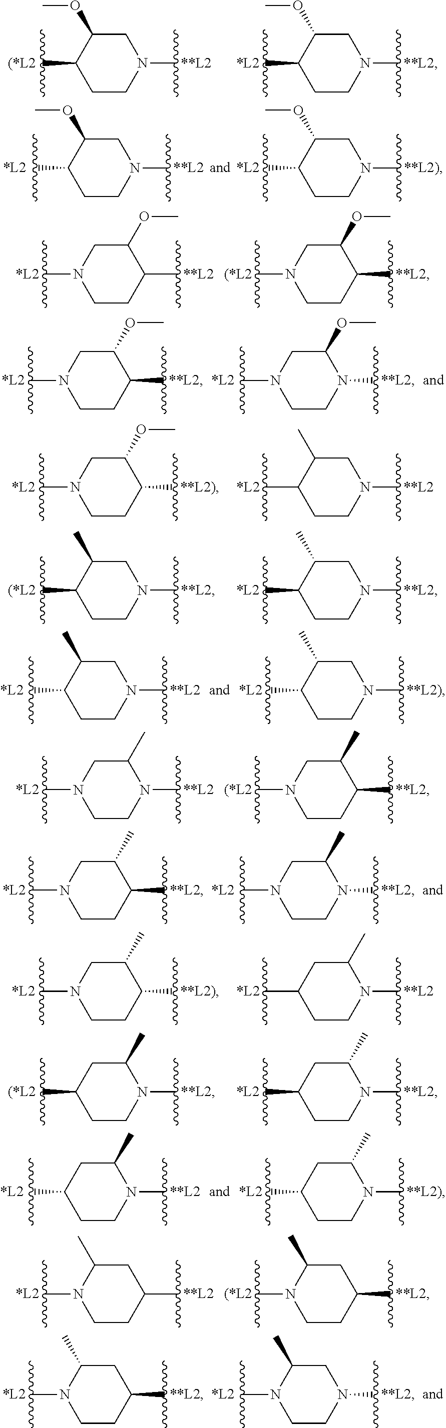

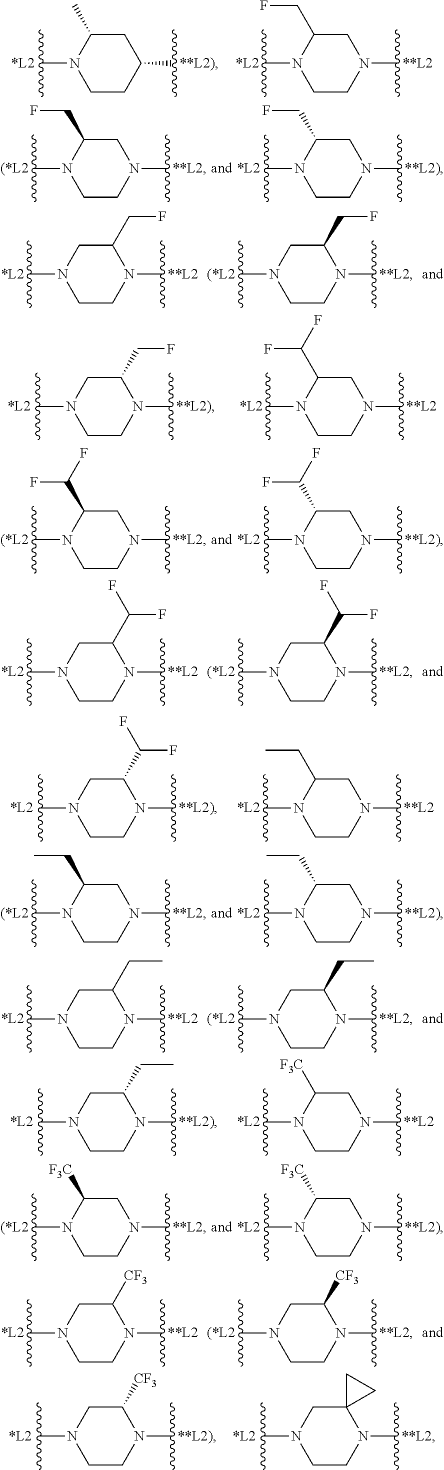

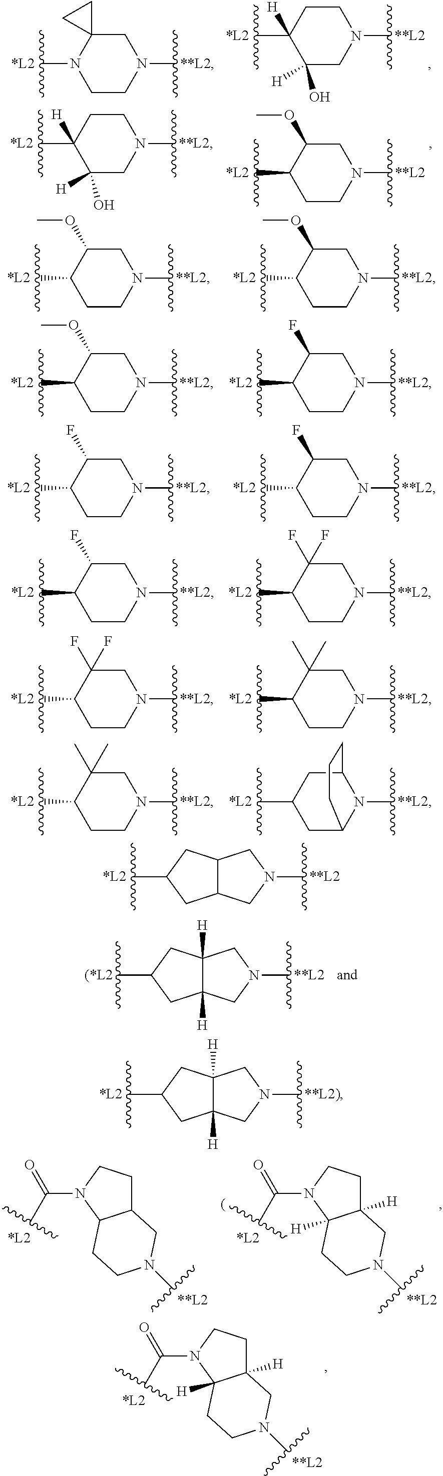

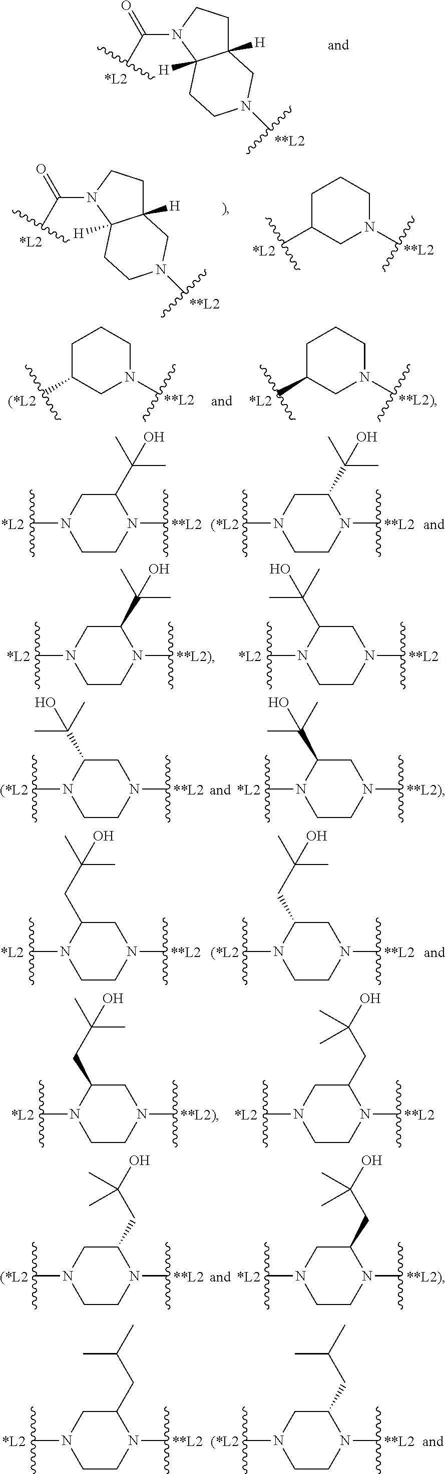

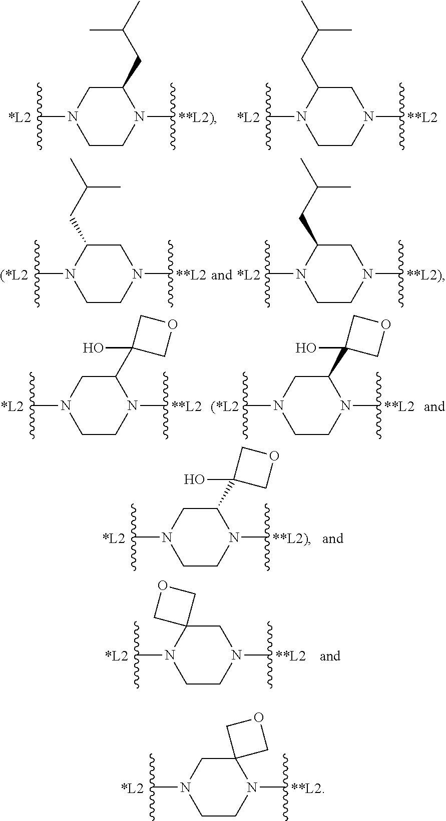

In one embodiment, L³ is selected from

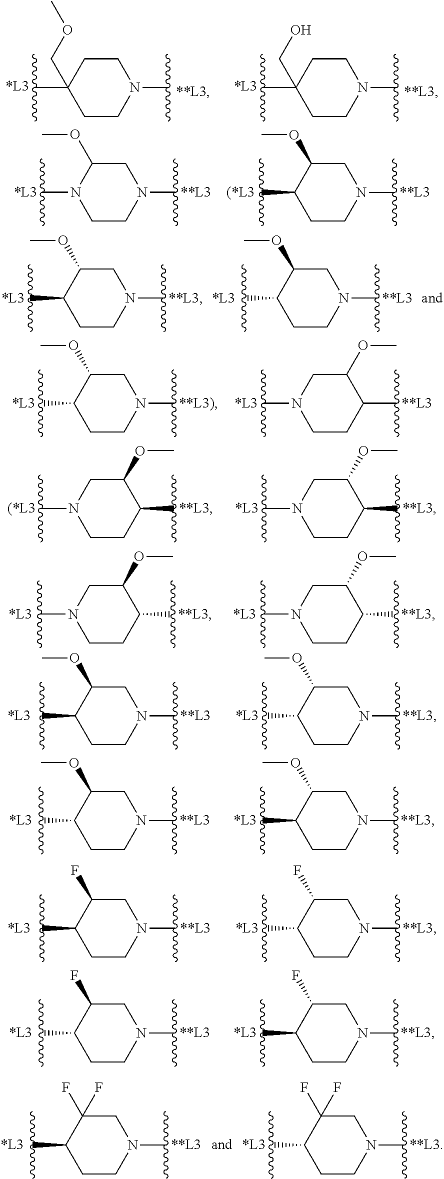

Aspect 23. The compound of any one of the preceding aspects, wherein

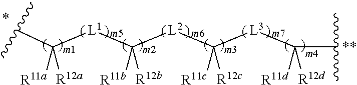

moiety is selected from

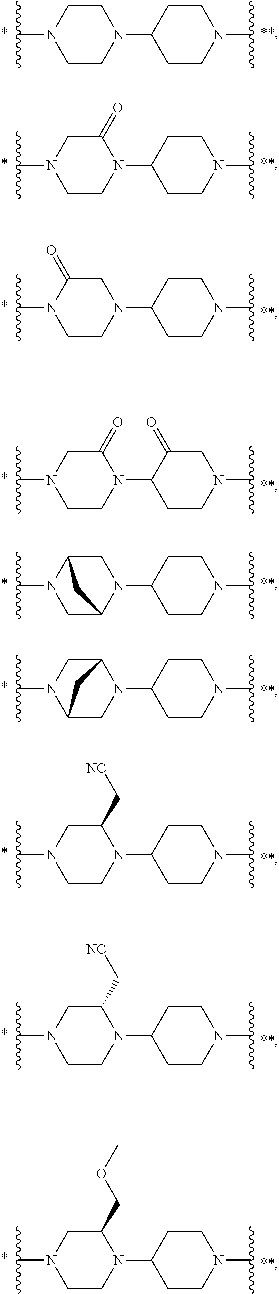

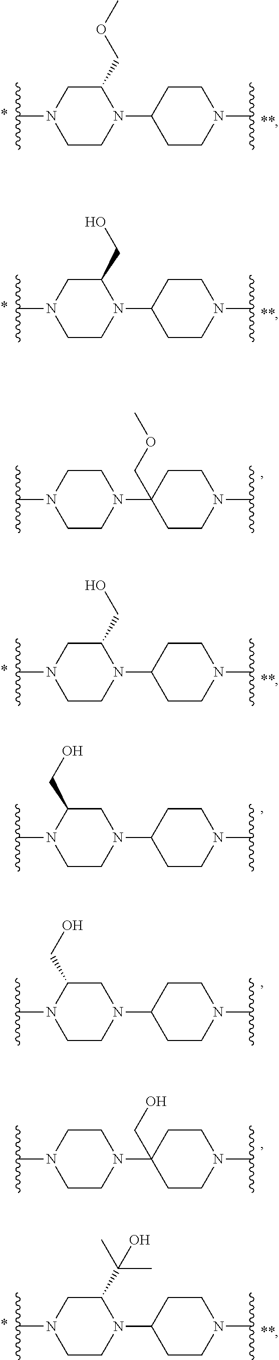

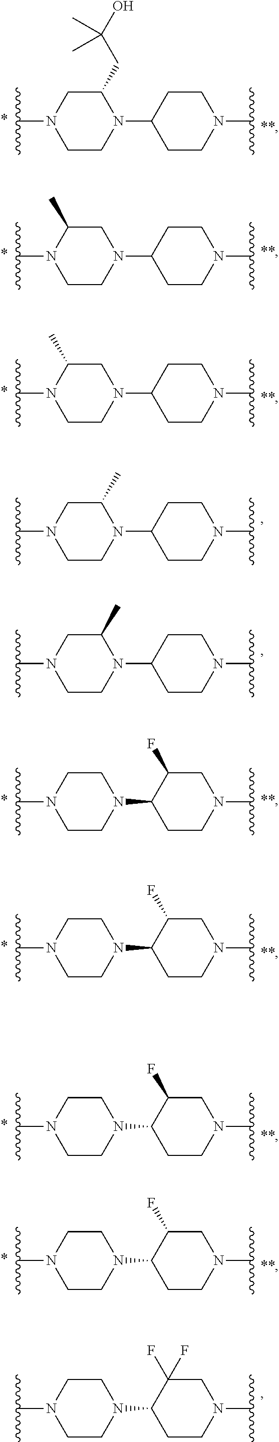

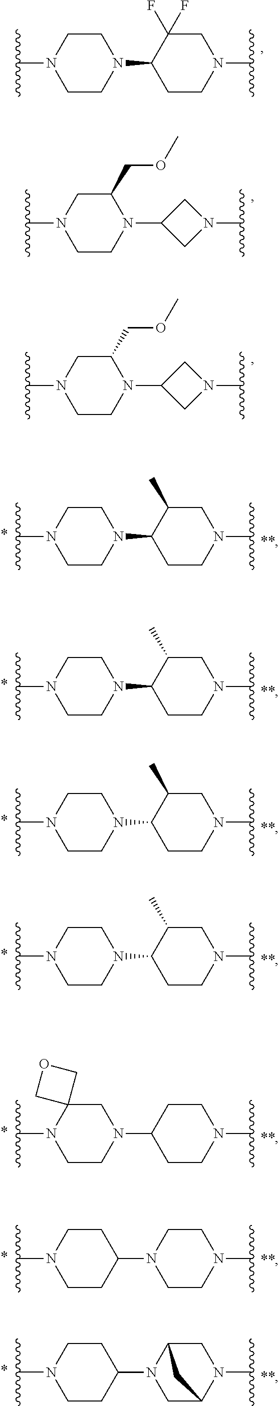

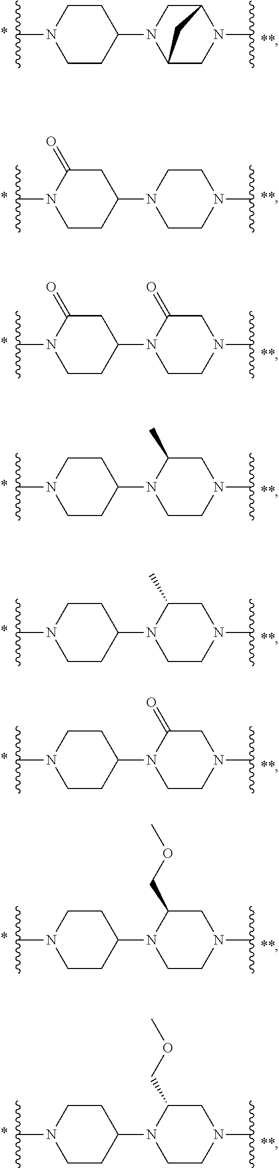

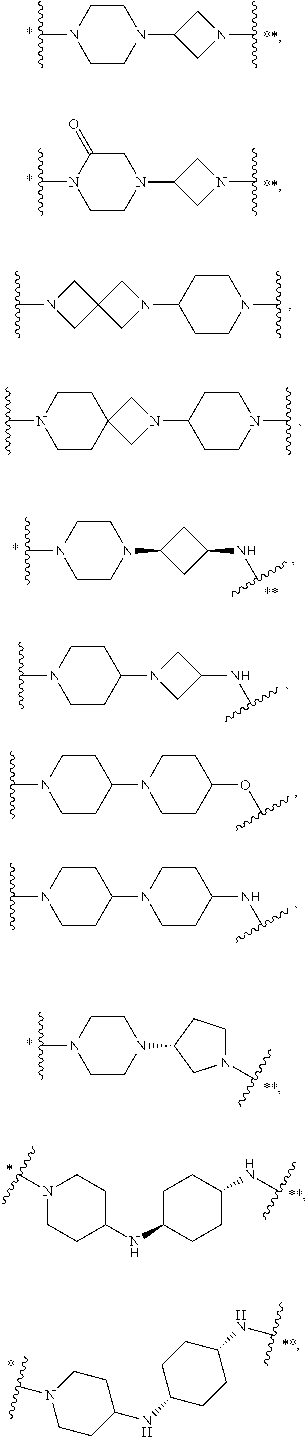

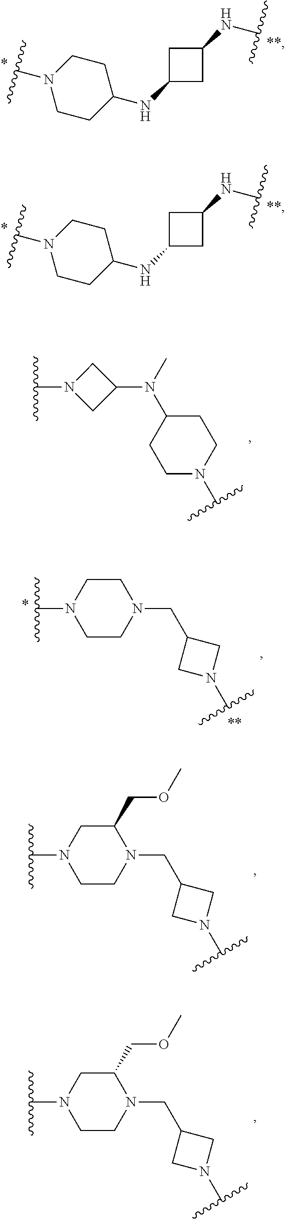 [SMILES: *N[C@H]1C[C@@H](NC2CCN(*)CC2)C1 ; *N[C@H]1C[C@H](NC2CCN(*)CC2)C1 ; *N1CCC(N(C)C2CN(*)C2)CC1 ; *N1CCN(CC2CN(*)C2)CC1 ; *N1CC(CN2CCN(*)C[C@H]2COC)C1 ; *N1CC(CN2CCN(*)C[C@@H]2COC)C1]

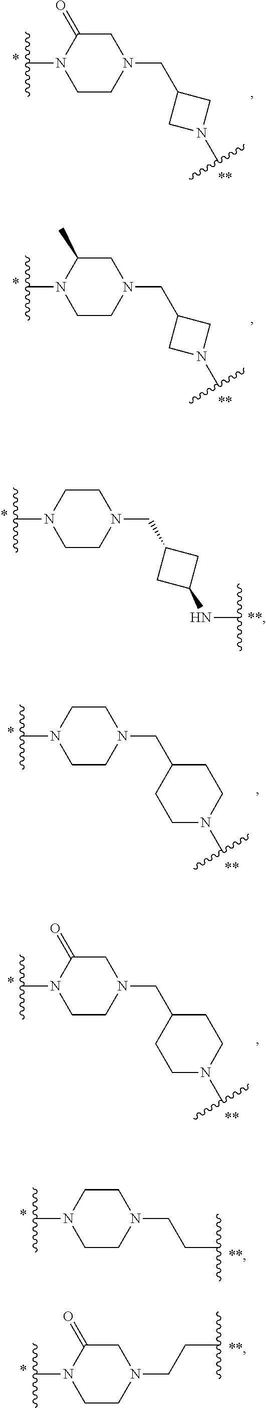

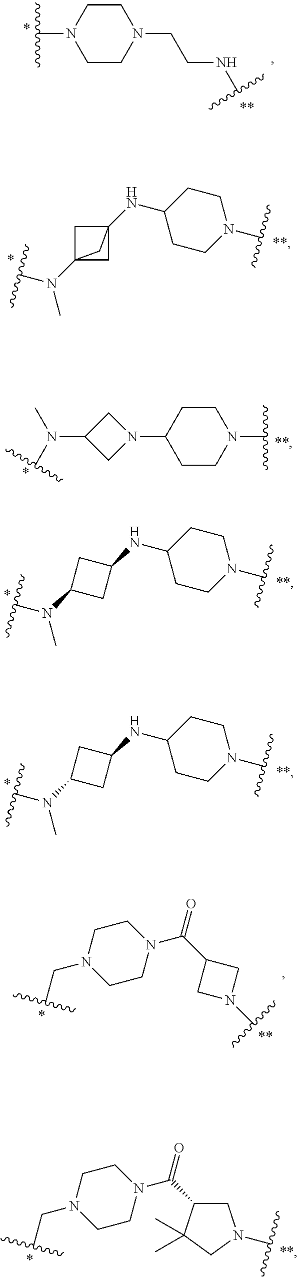

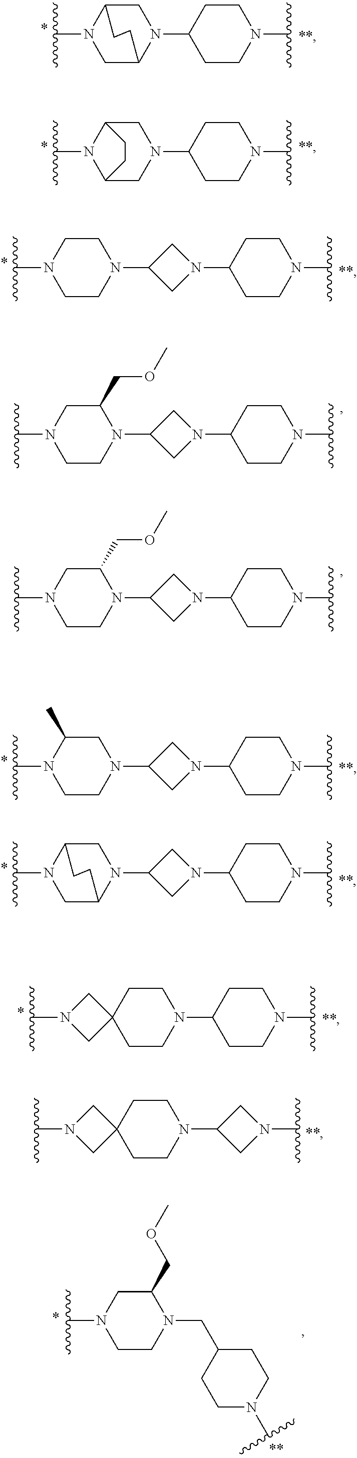

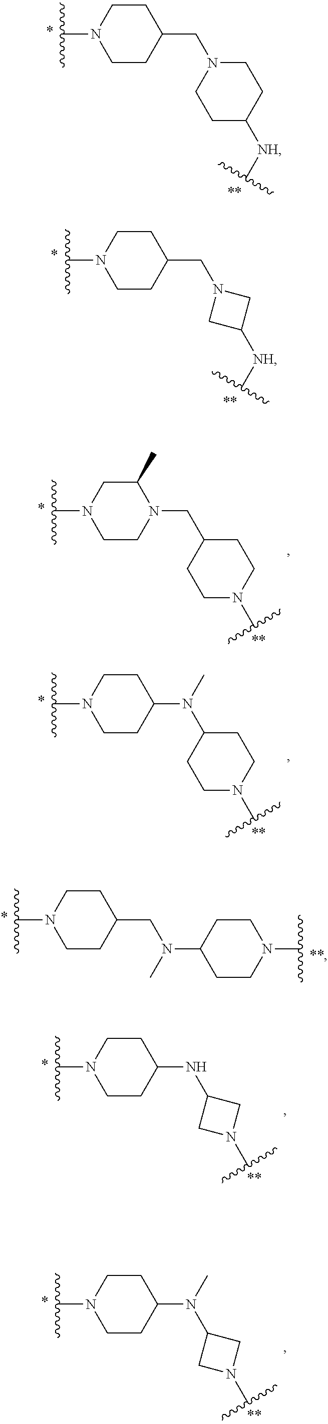

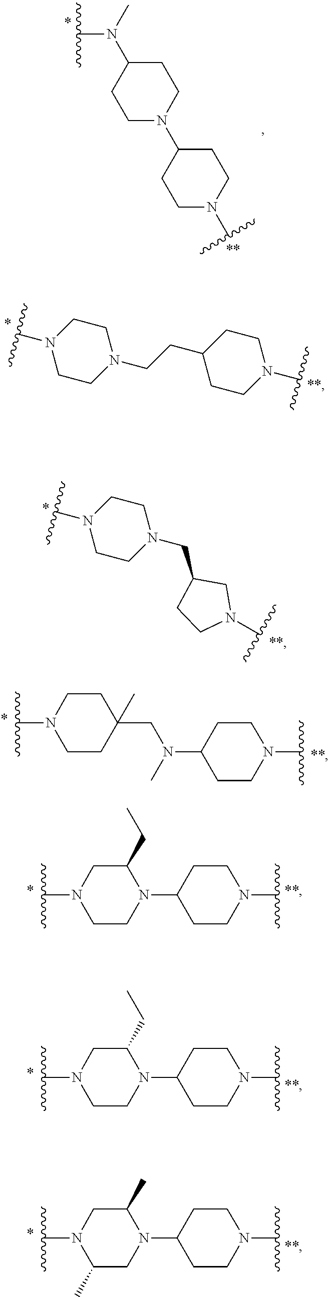

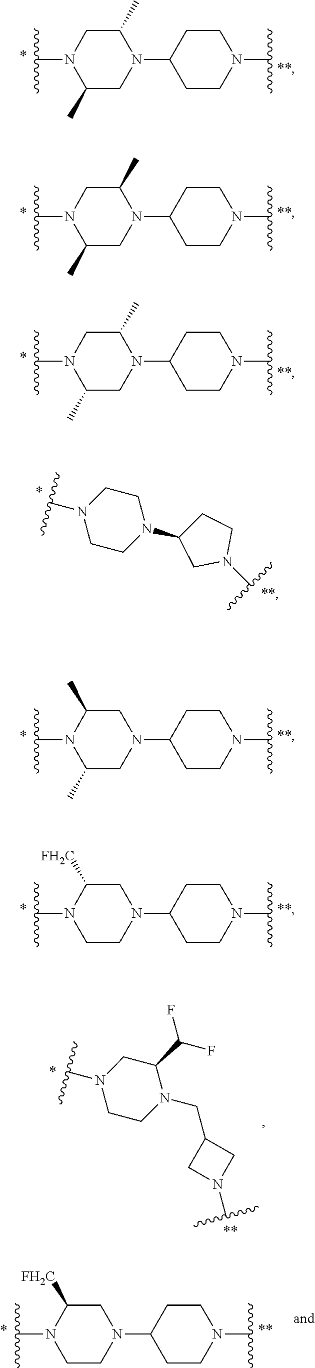

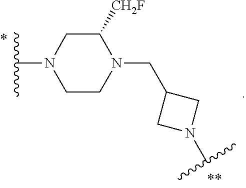

Aspect 24. The compound of any one of the preceding aspects, wherein L⁴ is independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— and —C(O)—; wherein at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one substituent selected from hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl;

In one embodiment, L⁴ is independently selected from a single bond.
In one embodiment, L⁴ is independently selected from —O—.
In one embodiment, L⁴ is independently selected from —NR^{a}—, wherein R^{a} is independently selected from hydrogen, methyl, ethyl or propyl.
In one embodiment, L⁴ is independently selected from —NH—.
In one embodiment, L⁴ is independently selected from —NCH₃—.
In one embodiment, L⁴ is independently selected from —(CR^{a}R^{b})ₙ₈—, wherein R^{a} and R^{b} are each independently selected from hydrogen, methyl, ethyl or propyl; n8 is 1 or 2.
In one embodiment, L⁴ is independently selected from —CH₂—.

Aspect 25. The compound of any one of the preceding aspects, wherein X⁷ is independently selected from —CR^{a}, and N;
R^{a} is independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl, wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.
   In one embodiment, X⁷ is independently selected from —CH, —C(CH₃), or N;
   In one embodiment, X⁷ is independently selected from —CH.

Aspect 26. The compound of any one of the preceding aspects, wherein X⁸ is independently selected from —NR^{a}—, —O—, —S— and —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl, wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent halogen, hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.
   In one embodiment, X⁸ is independently selected from —NH— and —CH₂—;
   In one embodiment, X⁸ is independently selected from —CH₂—.

Aspect 27. The compound of any one of the preceding aspects, wherein [symbol] is selected from

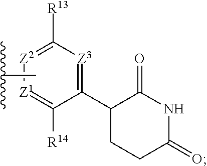

In one embodiment, [symbol] is selected from

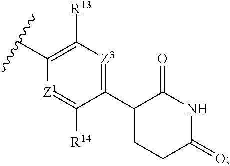

In one embodiment, [symbol] is selected from

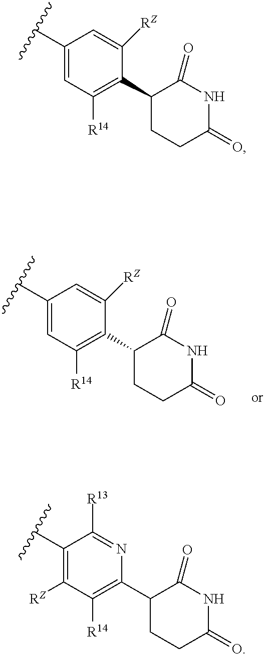

Aspect 28. The compound of any one of the preceding aspects, wherein at most one of Z¹, Z² and Z³ is N;

In one embodiment, Z¹, Z² and Z³ are each independently CR^{z};
In one embodiment, Z¹ is N, Z² and Z³ are each independently CR^{z}.

Aspect 29. The compound of any one of the preceding aspects, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl and CN; wherein each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one R^{Zc};
R^{Za} and R^{Zb} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl and 5- to 12-membered heteroaryl, wherein each of said hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl, or 5- to 12-membered heteroaryl.
   In one embodiment, R^{z} is selected from H, —CH₃, —C₂Hs, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.
   In one embodiment, R^{z} is H. In one embodiment, R^{z} is —CH₃. In one embodiment, R^{z} is F.

Aspect 30. The compound of any one of the preceding aspects, wherein R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, NR^{13a}COR^{13b}, —NR^{13a}CO₂R^{13b}, and —NR^{13a}SO₂R^{13b}; wherein each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13d}, —COR^{13c}, —CO₂R^{13c}, —CONR^{13c}R^{13a}, —NR^{13c}R^{13a}, —NR^{13c}COR^{13d}, —NR^{13c}CO₂R^{13d}, or —NR^{13c}SO₂R^{13a}.
R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl;
   In one embodiment, R¹³ and R¹⁴ are each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.
   In one embodiment, R¹³ and R¹⁴ are each independently selected from hydrogen, F, methyl.
   In one embodiment, R¹³ is independently selected from hydrogen, F, methyl.
   In one embodiment, R¹⁴ is independently selected from hydrogen, F, methyl.

Aspect 31. The compound of any one of the preceding aspects, wherein [symbol] is

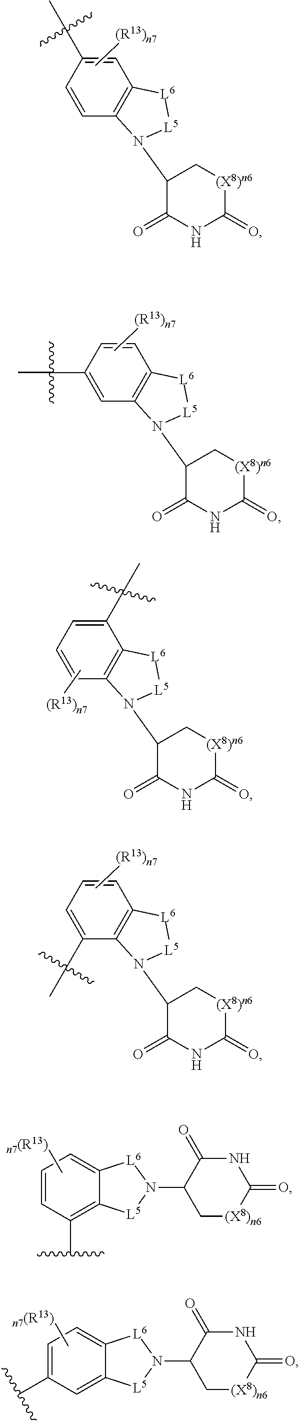

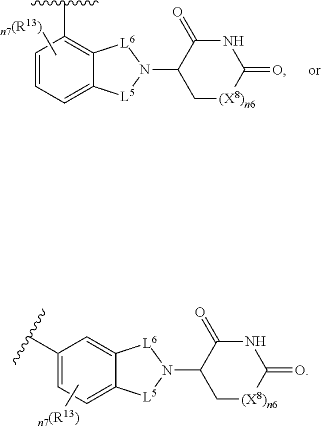

Aspect 32. The compound of any one of the preceding aspects, wherein L⁵ and L⁶ are each independently selected from a single bond, —O—, —NR^{a}—, —(CR^{a}R^{b})ₙ₈—, —O(CR^{a}R^{b})ₙ₈—, —NR^{a}(CR^{a}R^{b})ₙ₈— and —C(O)—;
X⁸ is —CR^{a}R^{b}—;
at each occurrence, R^{a} and R^{b} are each independently selected from hydrogen, hydroxy, —F, —Cl, —Br, —I, —CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl and 5- to 12-membered heteroaryl, wherein each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent hydroxy, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.
   In one embodiment, L⁵ and L⁶ are each independently a single bond, —O—, —NR^{a}—, —(CR^{a}R^{b})ₙ₈—, or —C(O)—;
wherein R^{a} and R^{b} are each independently selected from hydrogen and —C₁₋₃Alkyl; n8 is 1.
   In one embodiment, L⁵ and L⁶ are each independently a single bond,

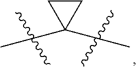 [SMILES: *C1(*)CC1]

—O—, —NH—, —NMe-, —N(CH₂CH₃)—, —CH₂—, —CHF—, —CF₂—, —C(CH₃)₂— or —CO—;
X⁸ is CH₂; and
n6 is 0 or 1.

Aspect 33. The compound of any one of the preceding aspects, wherein R¹³ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{13a}, —SO₂NR^{13a}R^{13b}, —COR^{13a}, —CO₂R^{13a}, —CONR^{13a}R^{13b}, —NR^{13a}R^{13b}, —NR^{13a}COR^{13b}, —NR^{13a}CO₂R^{13b}, and —NR^{13a}SO₂R^{13b}; wherein each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{13c}, —SO₂R^{13c}, —SO₂NR^{13c}R^{13a}, —COR^{13c}, —CO₂R^{13c}, —CONR^{13c}R^{13a}, —NR^{13c}R^{13a}, —NR^{13c}COR^{13d}, —NR^{13c}CO₂R^{13d}, or —NR^{13c}SO₂R^{13d};
at each occurrence, R^{13a}, R^{13b}, R^{13c} and R^{13d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl;
   In one embodiment, R¹³ is independently selected from hydrogen, F, Cl, Br, I, CN, —C₁-C₈alkyl, and —C₁-C₈alkoxy; preferably R¹³ is independently selected from hydrogen, F, Cl, Br, I, CN, -Me, -Et, —C₃H₇, —C₄H₉, —OMe, —OEt, —OC₃H₇ and —OC₄H₉;
n₇ is 0, 1 or 2.

Aspect 34. The compound of any one of the preceding aspects, wherein [symbol] is

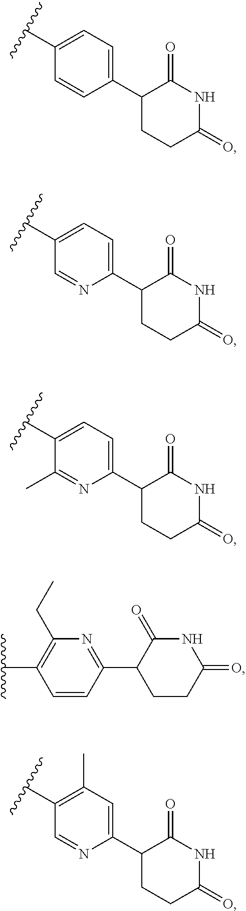 [SMILES: *c1ccc(C2CCC(=O)NC2=O)cc1 ; *c1ccc(C2CCC(=O)NC2=O)nc1 ; *c1ccc(C2CCC(=O)NC2=O)nc1C ; *c1ccc(C2CCC(=O)NC2=O)nc1CC ; *c1cnc(C2CCC(=O)NC2=O)cc1C]

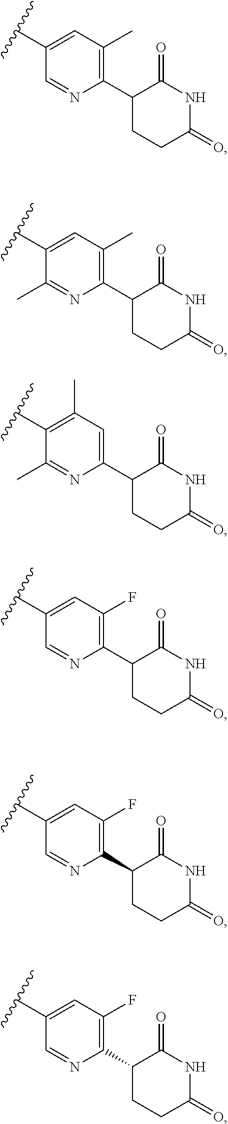 [SMILES: *c1cnc(C2CCC(=O)NC2=O)c(C)c1 ; *c1cc(C)c(C2CCC(=O)NC2=O)nc1C ; *c1c(C)cc(C2CCC(=O)NC2=O)nc1C ; *c1cnc(C2CCC(=O)NC2=O)c(F)c1 ; *c1cnc([C@@H]2CCC(=O)NC2=O)c(F)c1 ; *c1cnc([C@H]2CCC(=O)NC2=O)c(F)c1]

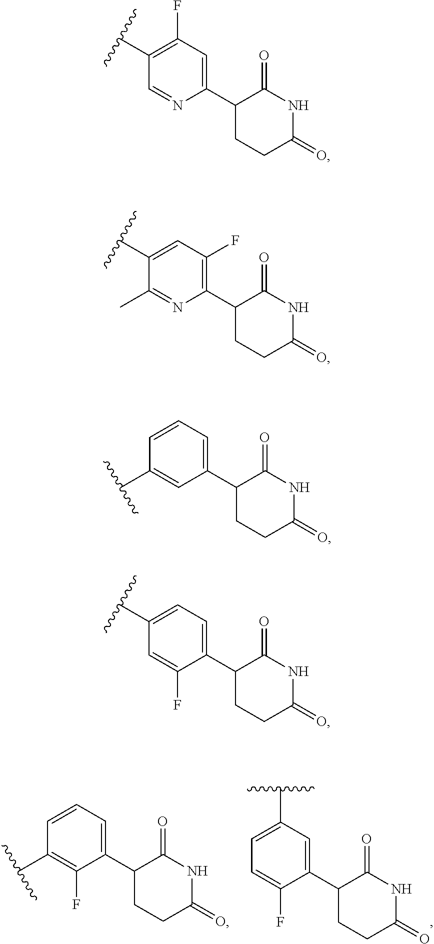 [SMILES: *c1cnc(C2CCC(=O)NC2=O)cc1F ; *c1cc(F)c(C2CCC(=O)NC2=O)nc1C ; *c1cccc(C2CCC(=O)NC2=O)c1 ; *c1ccc(C2CCC(=O)NC2=O)c(F)c1 ; *c1cccc(C2CCC(=O)NC2=O)c1F ; *c1ccc(F)c(C2CCC(=O)NC2=O)c1]

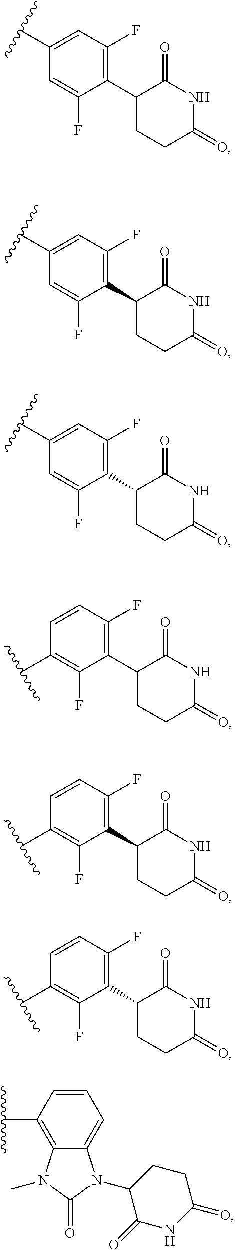

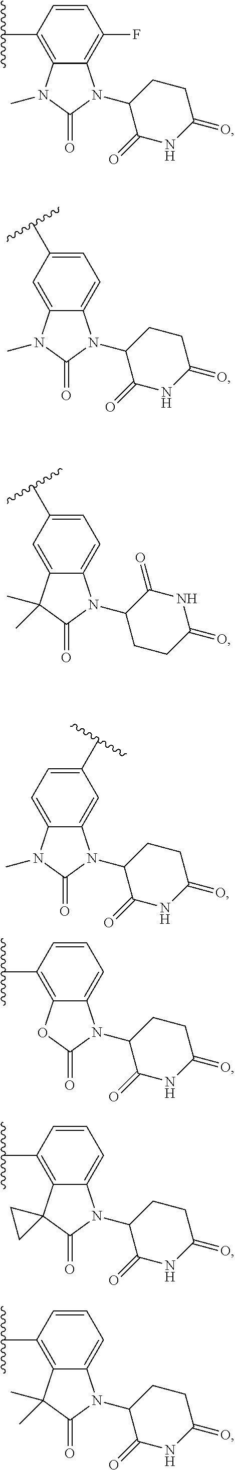

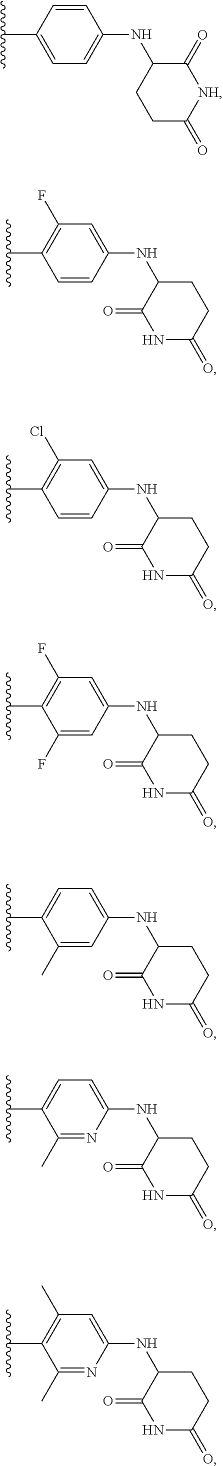

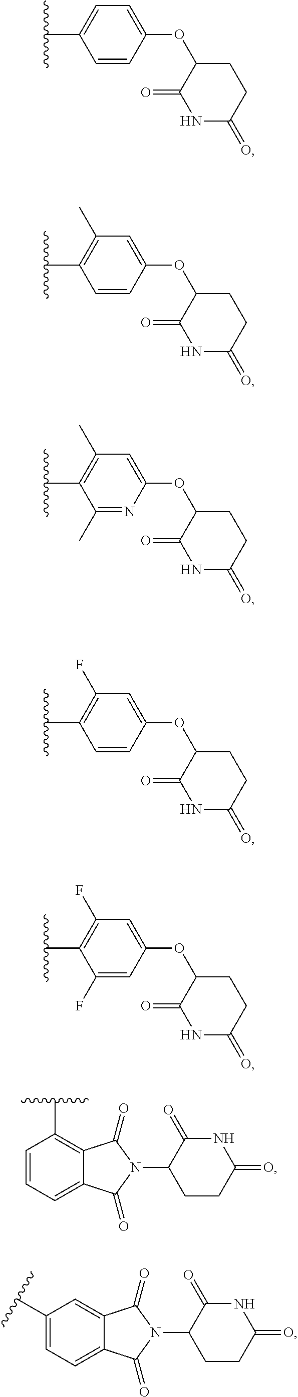

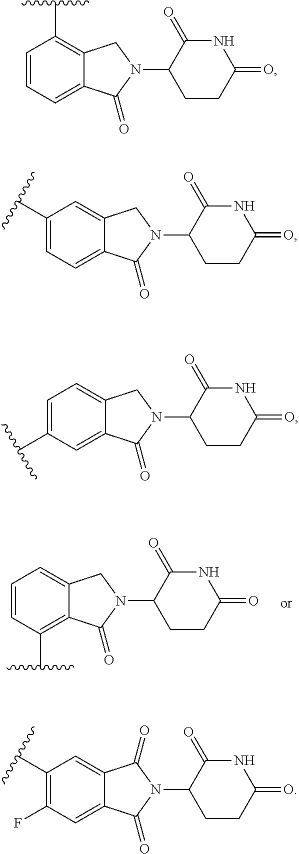 [SMILES: *c1cccc2c1CN(C1CCC(=O)NC1=O)C2=O ; *c1ccc2c(c1)CN(C1CCC(=O)NC1=O)C2=O ; *c1ccc2c(c1)C(=O)N(C1CCC(=O)NC1=O)C2 ; *c1cccc2c1C(=O)N(C1CCC(=O)NC1=O)C2 ; *c1cc2c(cc1F)C(=O)N(C1CCC(=O)NC1=O)C2=O]

Aspect 35. The compound of any one of the preceding aspects is selected from

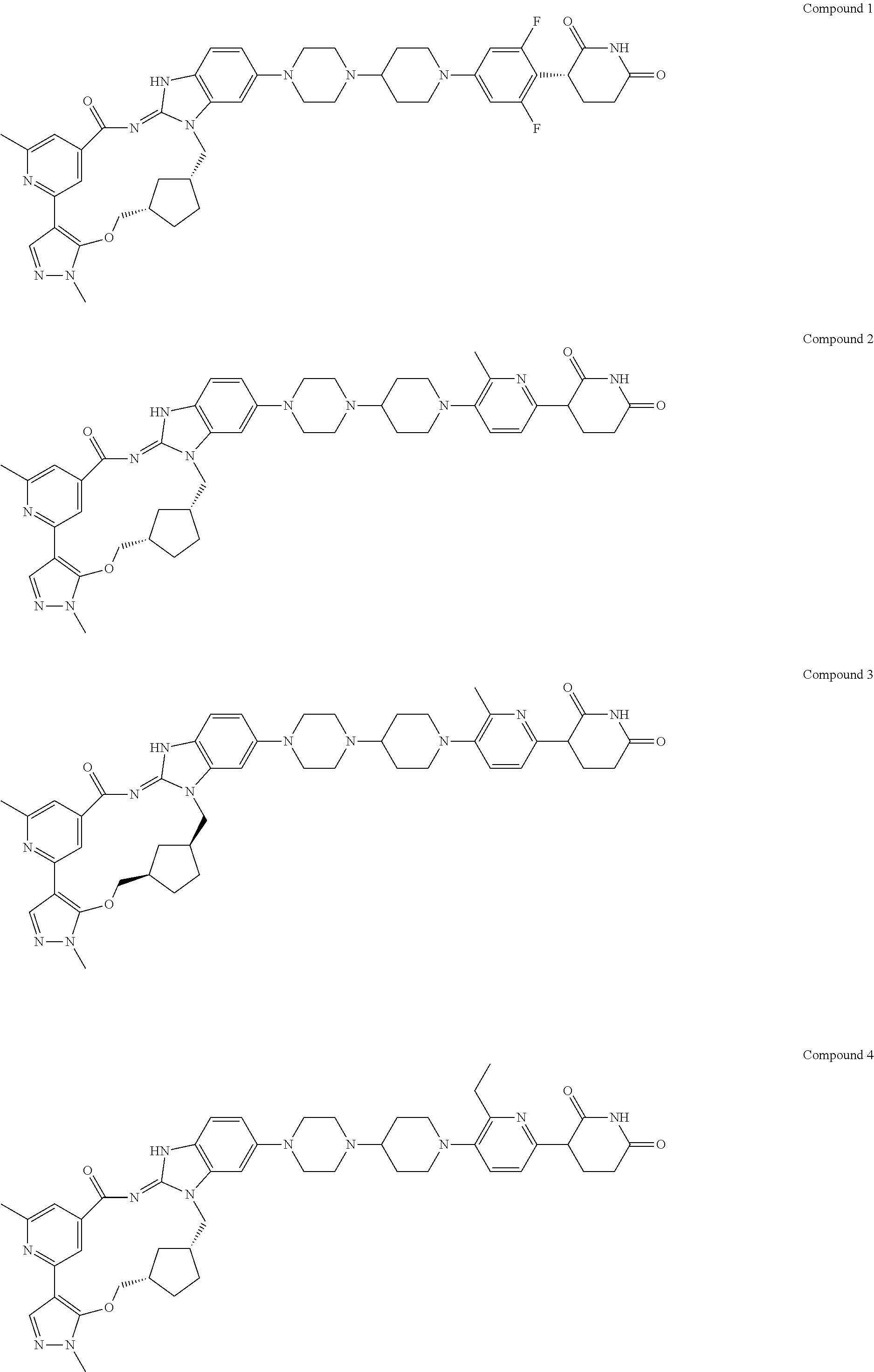 [SMILES: Cc1cc2cc(n1)-c1cnn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)CC3)cc21 ; Cc1cc2cc(n1)-c1cnn(C)c1OC[C@H]1CC[C@H](C1)CN1/C(=N/C2=O)Nc2ccc(N3CCN(C4CCN(c5ccc(C6CCC(=O)NC6=O)nc5C)CC4)CC3)cc21 ; Cc1cc2cc(n1)-c1cnn(C)c1OC[C@@H]1CC[C@@H](C1)CN1/C(=N/C2=O)Nc2ccc(N3CCN(C4CCN(c5ccc(C6CCC(=O)NC6=O)nc5C)CC4)CC3)cc21 ; CCc1nc(C2CCC(=O)NC2=O)ccc1N1CCC(N2CCN(c3ccc4c(c3)N3C[C@@H]5CC[C@H](COc6c(cnn6C)-c6cc(cc(C)n6)C(=O)/N=C/3N4)C5)CC2)CC1]

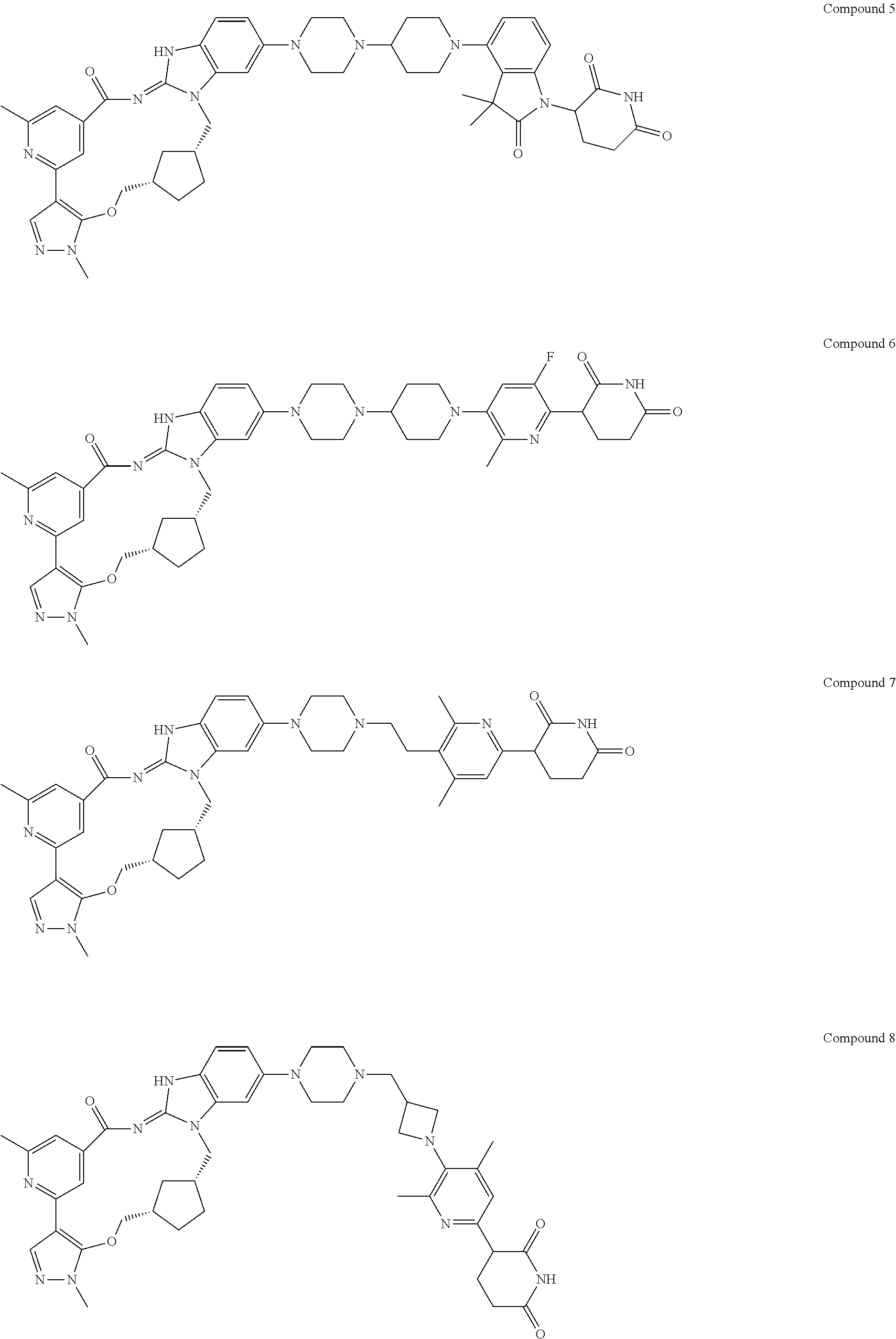 [SMILES: Cc1cc2cc(n1)-c1cnn(C)c1OC[C@H]1CC[C@H](C1)CN1/C(=N/C2=O)Nc2ccc(N3CCN(C4CCN(c5cccc6c5C(C)(C)C(=O)N6C5CCC(=O)NC5=O)CC4)CC3)cc21 ; Cc1cc2cc(n1)-c1cnn(C)c1OC[C@H]1CC[C@H](C1)CN1/C(=N/C2=O)Nc2ccc(N3CCN(C4CCN(c5cc(F)c(C6CCC(=O)NC6=O)nc5C)CC4)CC3)cc21 ; Cc1cc2cc(n1)-c1cnn(C)c1OC[C@H]1CC[C@H](C1)CN1/C(=N/C2=O)Nc2ccc(N3CCN(CCc4c(C)cc(C5CCC(=O)NC5=O)nc4C)CC3)cc21 ; Cc1cc2cc(n1)-c1cnn(C)c1OC[C@H]1CC[C@H](C1)CN1/C(=N/C2=O)Nc2ccc(N3CCN(CC4CN(c5c(C)cc(C6CCC(=O)NC6=O)nc5C)C4)CC3)cc21]

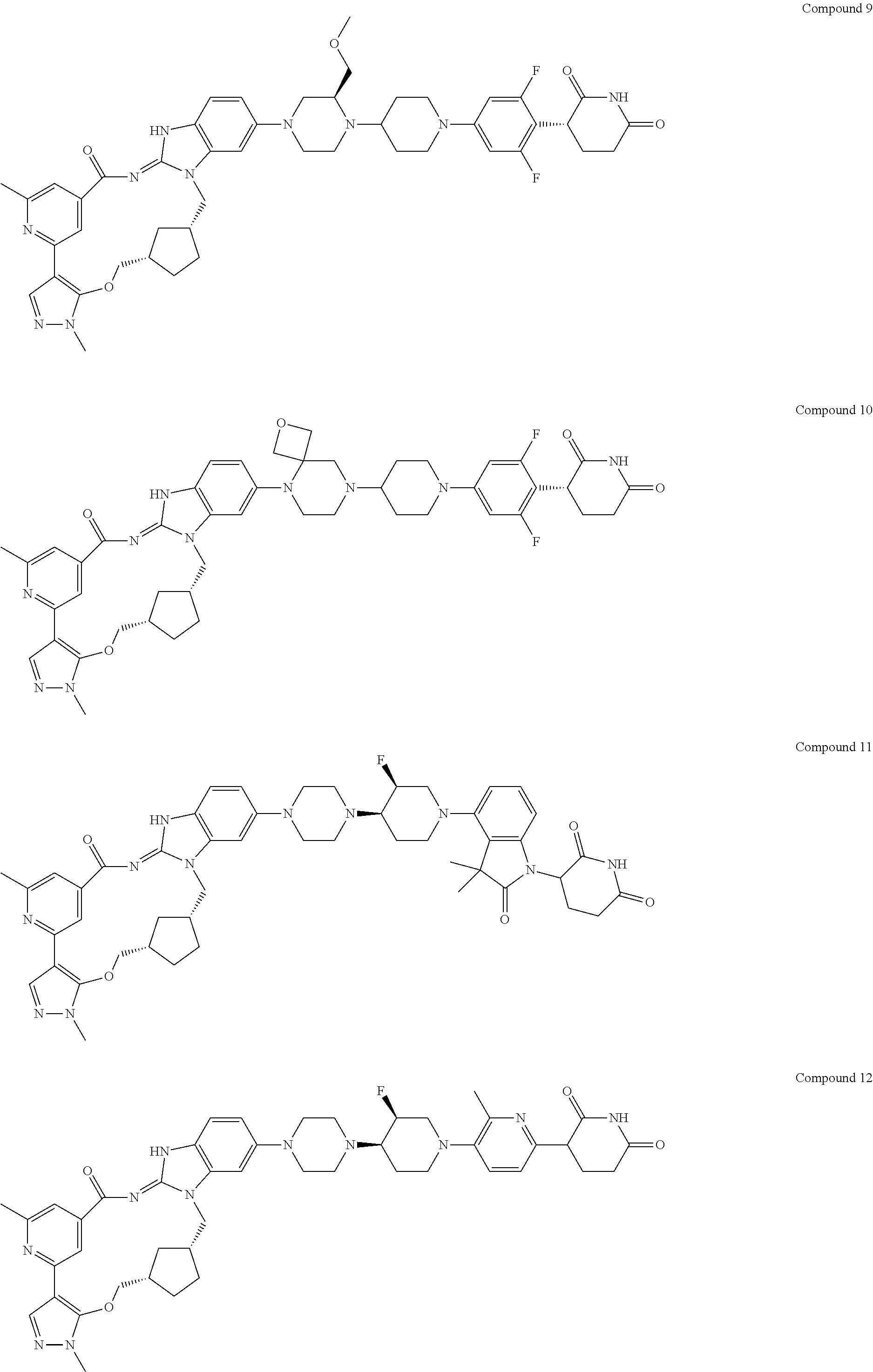

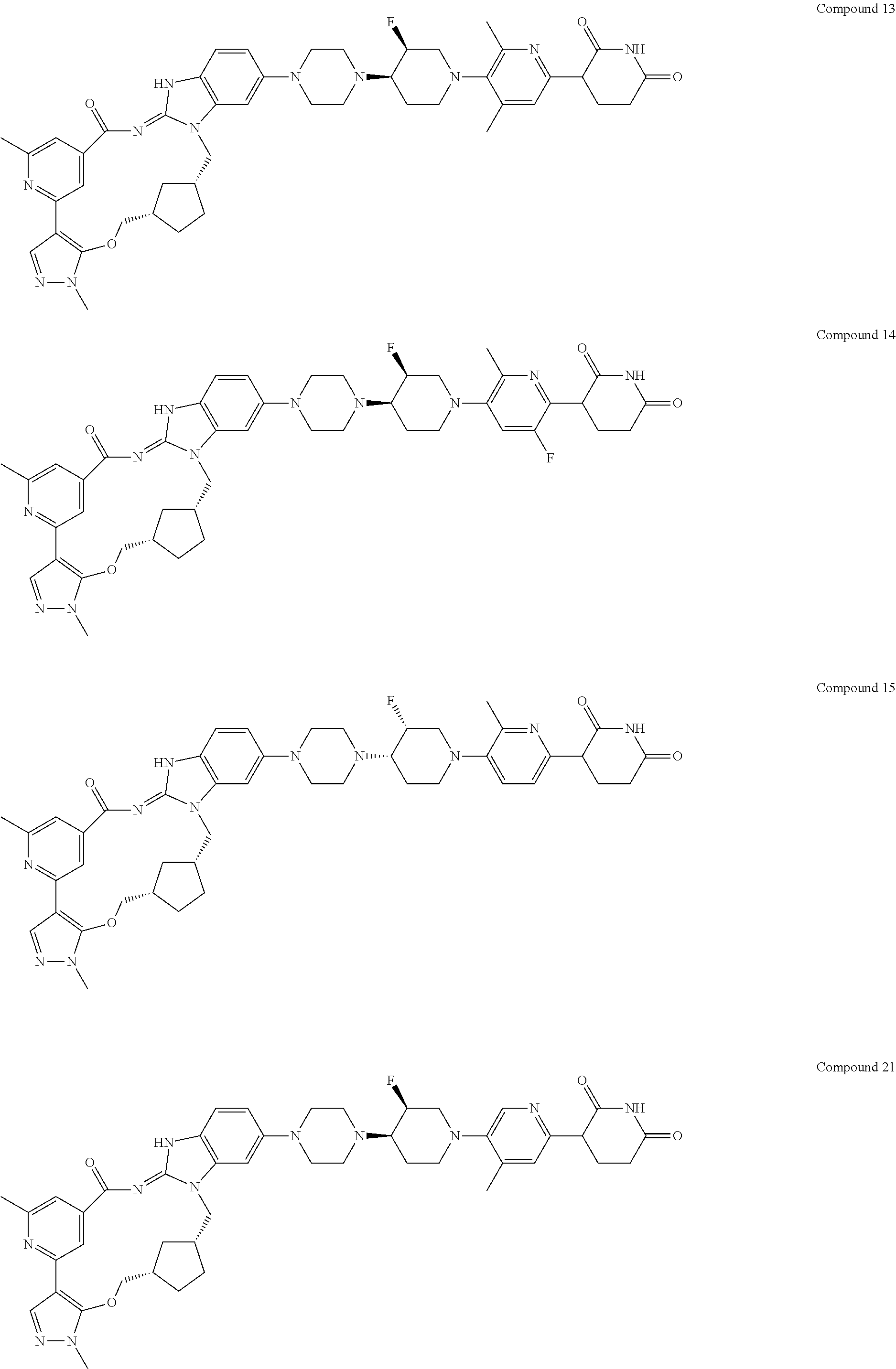

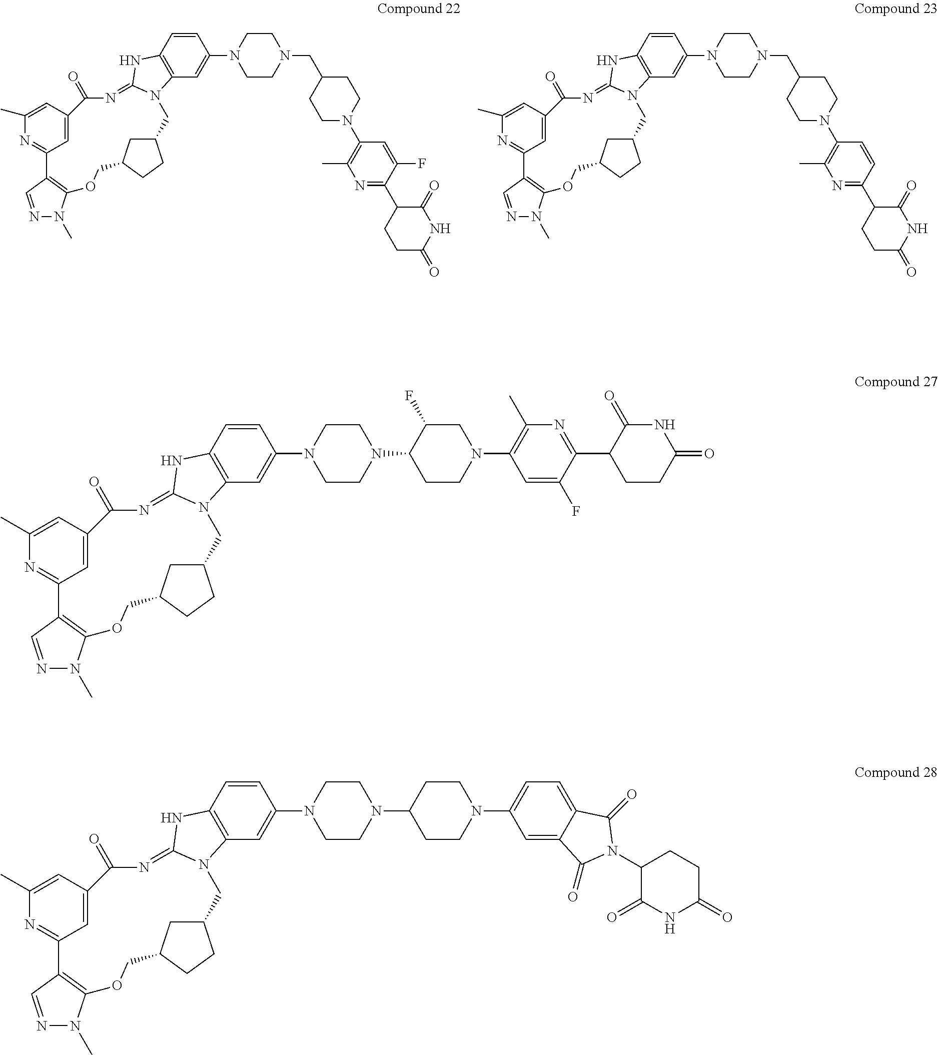 [SMILES: Cc1cc2cc(n1)-c1cnn(C)c1OC[C@H]1CC[C@H](C1)CN1/C(=N/C2=O)Nc2ccc(N3CCN(CC4CCN(c5cc(F)c(C6CCC(=O)NC6=O)nc5C)CC4)CC3)cc21 ; Cc1cc2cc(n1)-c1cnn(C)c1OC[C@H]1CC[C@H](C1)CN1/C(=N/C2=O)Nc2ccc(N3CCN(CC4CCN(c5ccc(C6CCC(=O)NC6=O)nc5C)CC4)CC3)cc21 ; Cc1cc2cc(n1)-c1cnn(C)c1OC[C@H]1CC[C@H](C1)CN1/C(=N/C2=O)Nc2ccc(N3CCN([C@H]4CCN(c5cc(F)c(C6CCC(=O)NC6=O)nc5C)C[C@H]4F)CC3)cc21 ; Cc1cc2cc(n1)-c1cnn(C)c1OC[C@H]1CC[C@H](C1)CN1/C(=N/C2=O)Nc2ccc(N3CCN(C4CCN(c5ccc6c(c5)C(=O)N(C5CCC(=O)NC5=O)C6=O)CC4)CC3)cc21]

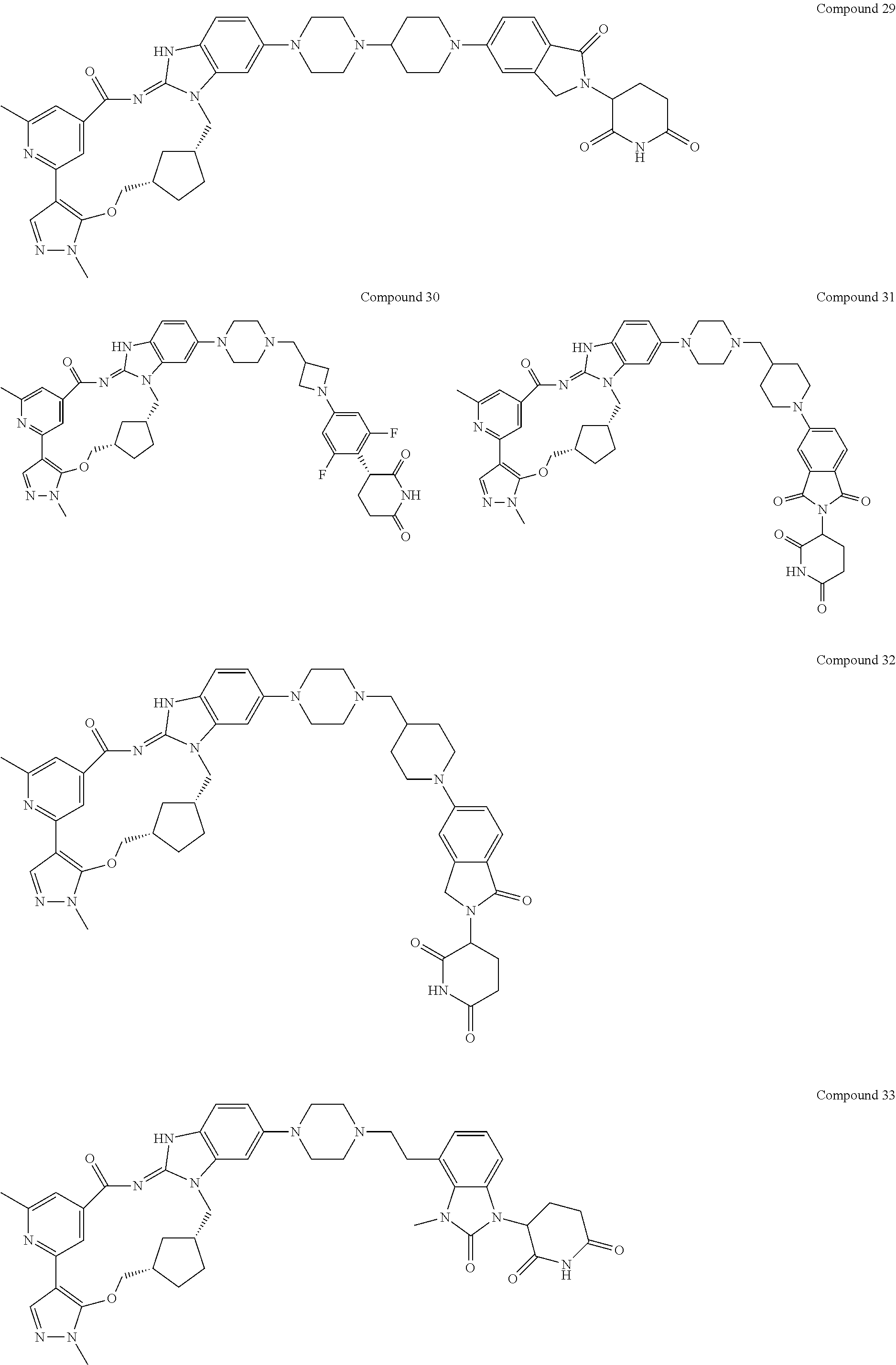

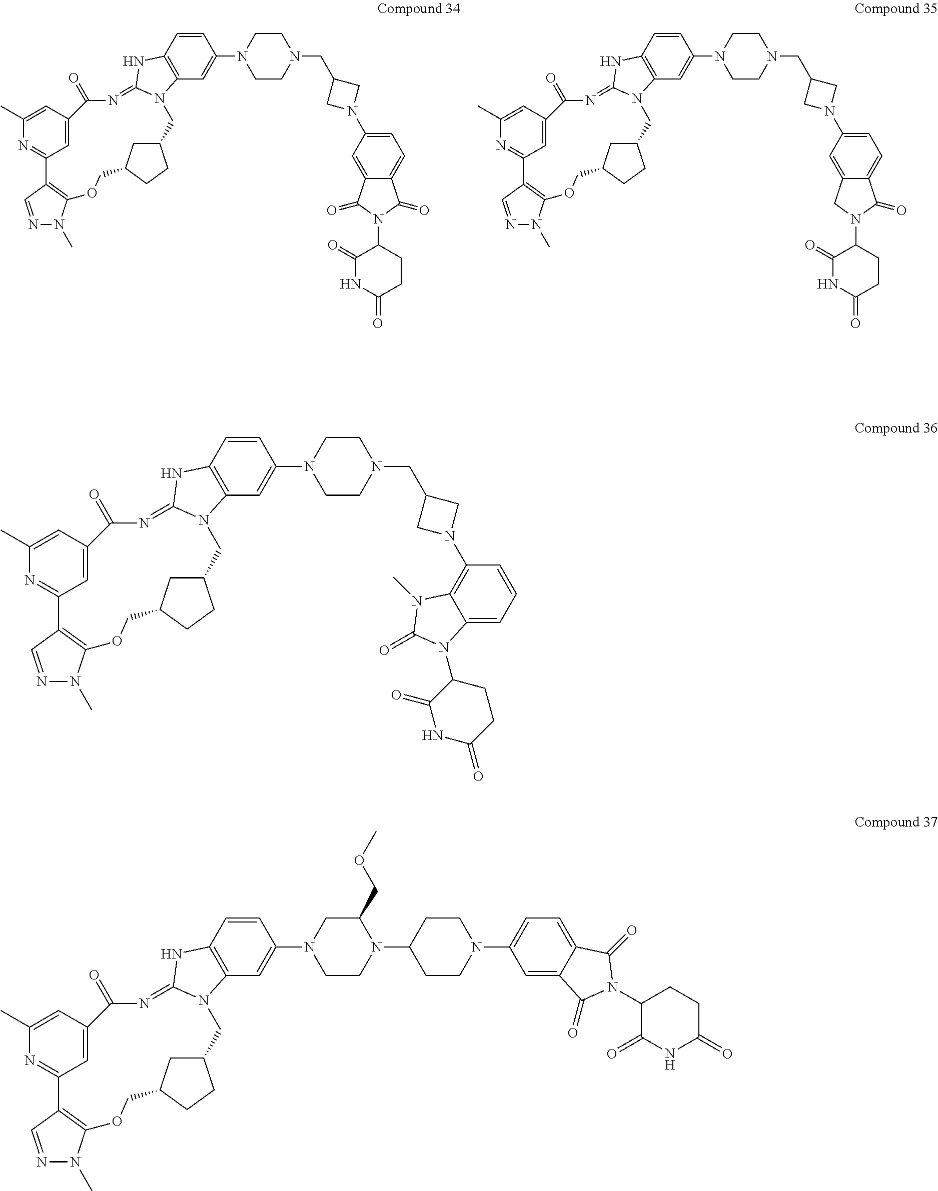

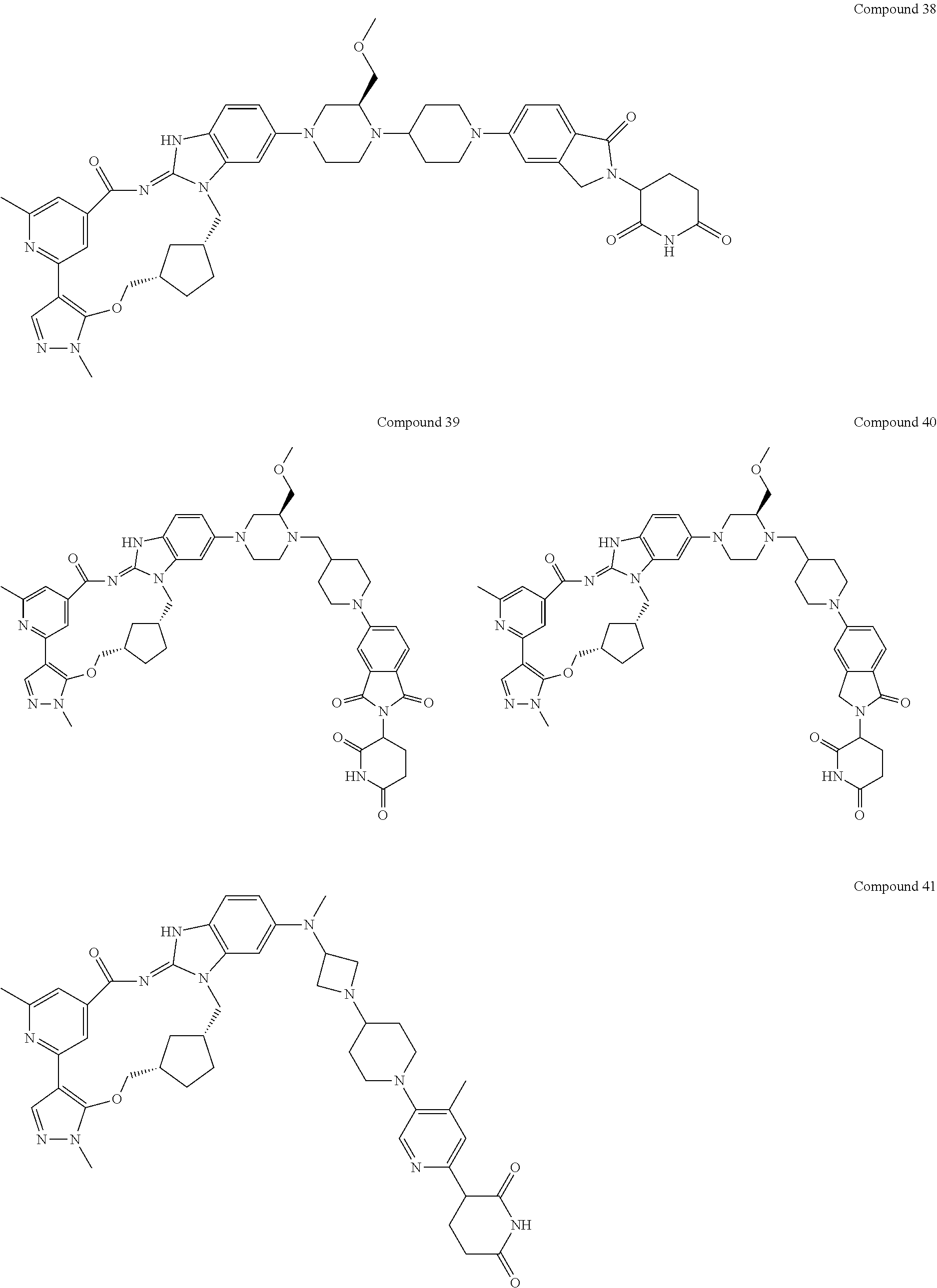

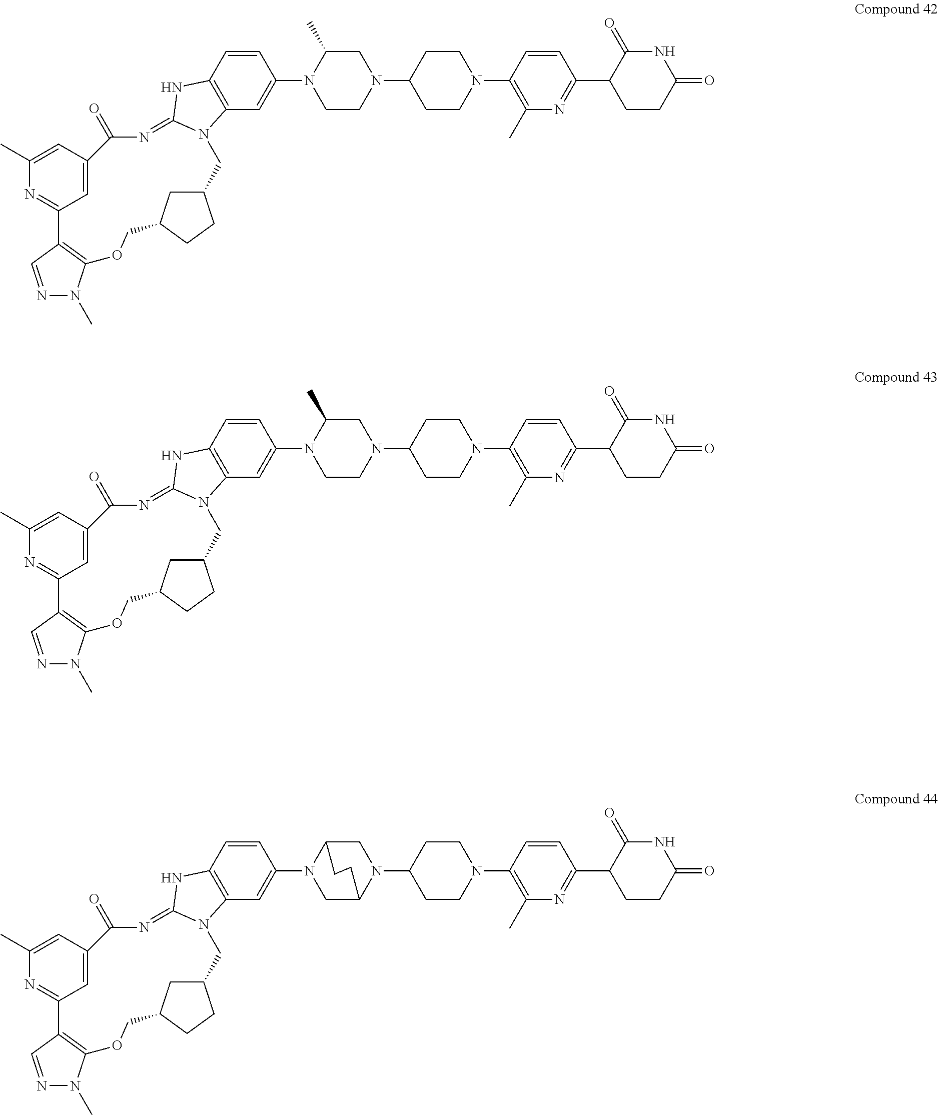 [SMILES: Cc1cc2cc(n1)-c1cnn(C)c1OC[C@H]1CC[C@H](C1)CN1/C(=N/C2=O)Nc2ccc(N3CCN(C4CCN(c5ccc(C6CCC(=O)NC6=O)nc5C)CC4)C[C@H]3C)cc21 ; Cc1cc2cc(n1)-c1cnn(C)c1OC[C@H]1CC[C@H](C1)CN1/C(=N/C2=O)Nc2ccc(N3CCN(C4CCN(c5ccc(C6CCC(=O)NC6=O)nc5C)CC4)C[C@@H]3C)cc21 ; Cc1cc2cc(n1)-c1cnn(C)c1OC[C@H]1CC[C@H](C1)CN1/C(=N/C2=O)Nc2ccc(N3CC4CCC3CN4C3CCN(c4ccc(C5CCC(=O)NC5=O)nc4C)CC3)cc21]

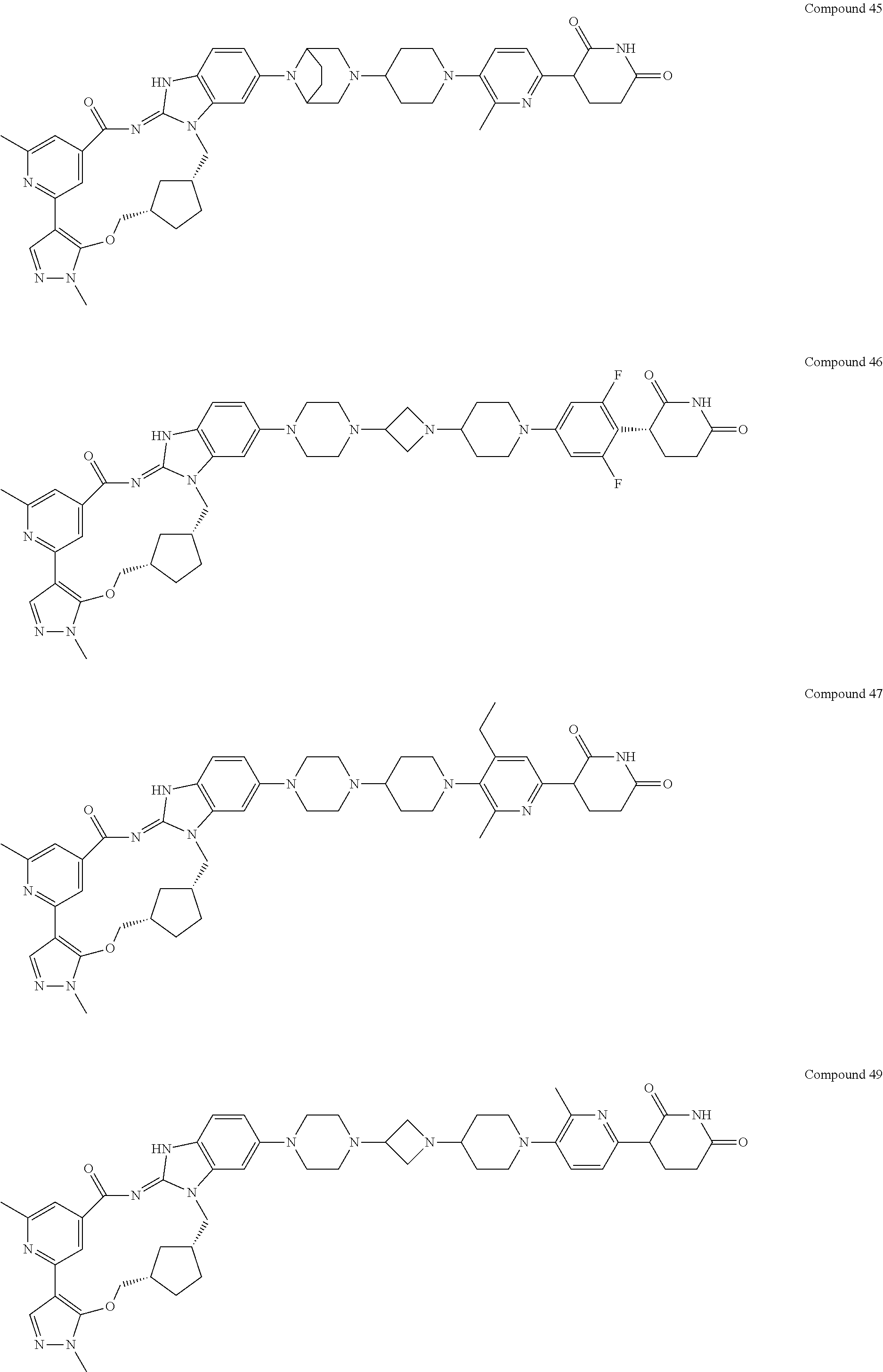 [SMILES: Cc1cc2cc(n1)-c1cnn(C)c1OC[C@H]1CC[C@H](C1)CN1/C(=N/C2=O)Nc2ccc(N3C4CCC3CN(C3CCN(c5ccc(C6CCC(=O)NC6=O)nc5C)CC3)C4)cc21 ; Cc1cc2cc(n1)-c1cnn(C)c1OC[C@H]1CC[C@H](C1)CN1/C(=N/C2=O)Nc2ccc(N3CCN(C4CN(C5CCN(c6cc(F)c([C@H]7CCC(=O)NC7=O)c(F)c6)CC5)C4)CC3)cc21 ; CCc1cc(C2CCC(=O)NC2=O)nc(C)c1N1CCC(N2CCN(c3ccc4c(c3)N3C[C@@H]5CC[C@H](COc6c(cnn6C)-c6cc(cc(C)n6)C(=O)/N=C/3N4)C5)CC2)CC1 ; Cc1cc2cc(n1)-c1cnn(C)c1OC[C@H]1CC[C@H](C1)CN1/C(=N/C2=O)Nc2ccc(N3CCN(C4CN(C5CCN(c6ccc(C7CCC(=O)NC7=O)nc6C)CC5)C4)CC3)cc21]

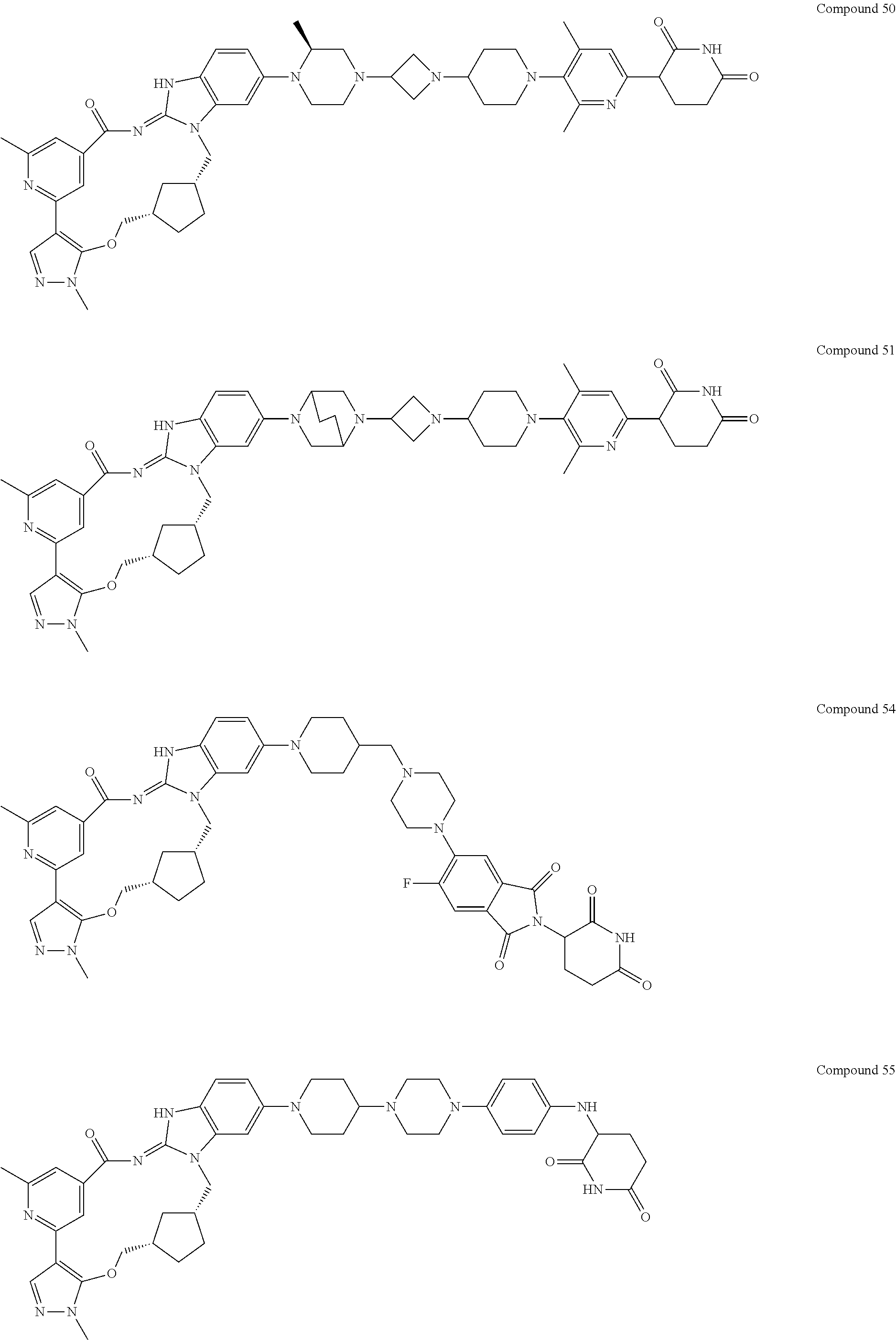

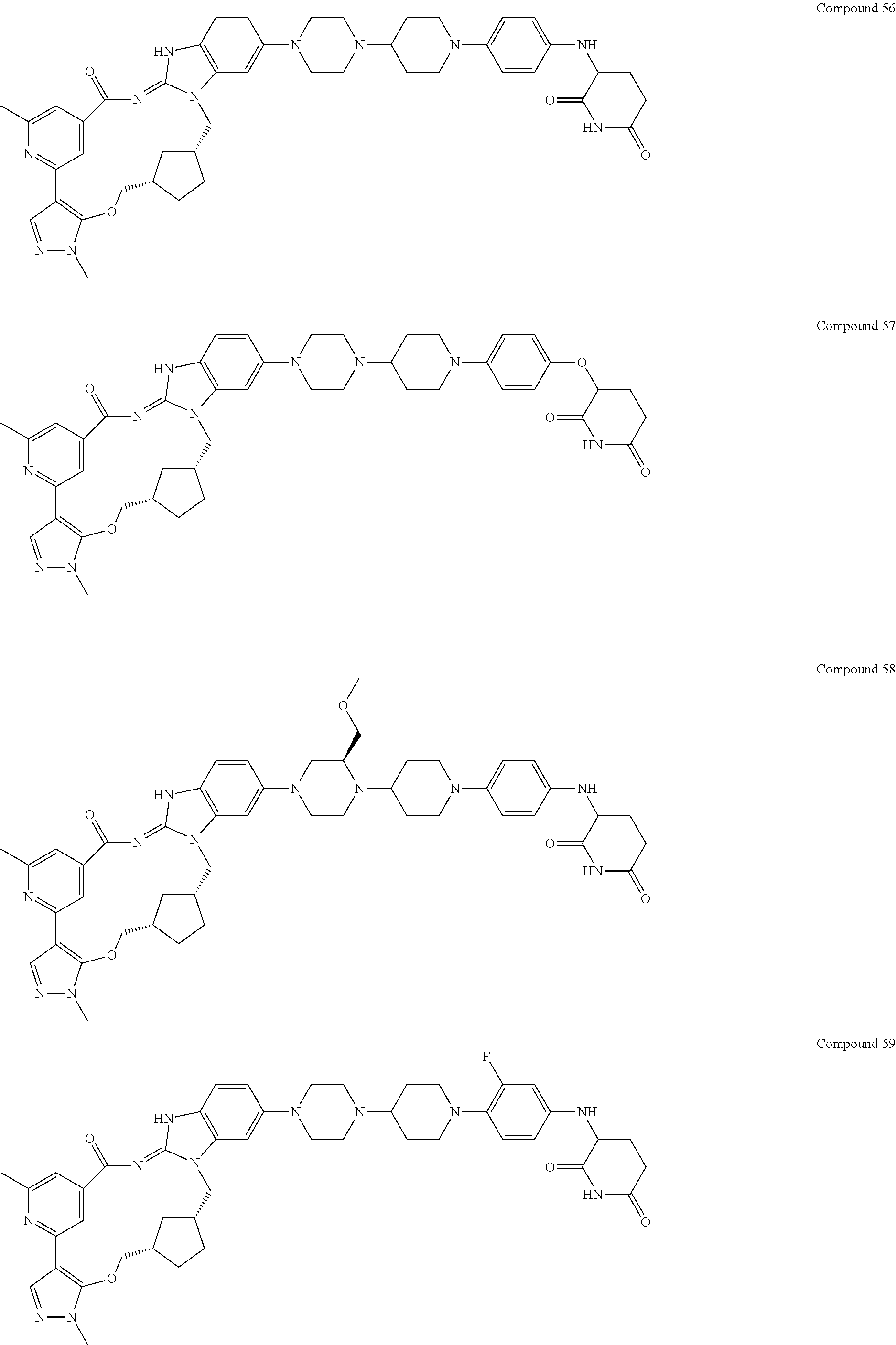 [SMILES: Cc1cc2cc(n1)-c1cnn(C)c1OC[C@H]1CC[C@H](C1)CN1/C(=N/C2=O)Nc2ccc(N3CCN(C4CCN(c5ccc(NC6CCC(=O)NC6=O)cc5)CC4)CC3)cc21 ; Cc1cc2cc(n1)-c1cnn(C)c1OC[C@H]1CC[C@H](C1)CN1/C(=N/C2=O)Nc2ccc(N3CCN(C4CCN(c5ccc(OC6CCC(=O)NC6=O)cc5)CC4)CC3)cc21 ; COC[C@@H]1CN(c2ccc3c(c2)N2C[C@@H]4CC[C@H](COc5c(cnn5C)-c5cc(cc(C)n5)C(=O)/N=C/2N3)C4)CCN1C1CCN(c2ccc(NC3CCC(=O)NC3=O)cc2)CC1 ; Cc1cc2cc(n1)-c1cnn(C)c1OC[C@H]1CC[C@H](C1)CN1/C(=N/C2=O)Nc2ccc(N3CCN(C4CCN(c5ccc(NC6CCC(=O)NC6=O)cc5F)CC4)CC3)cc21]

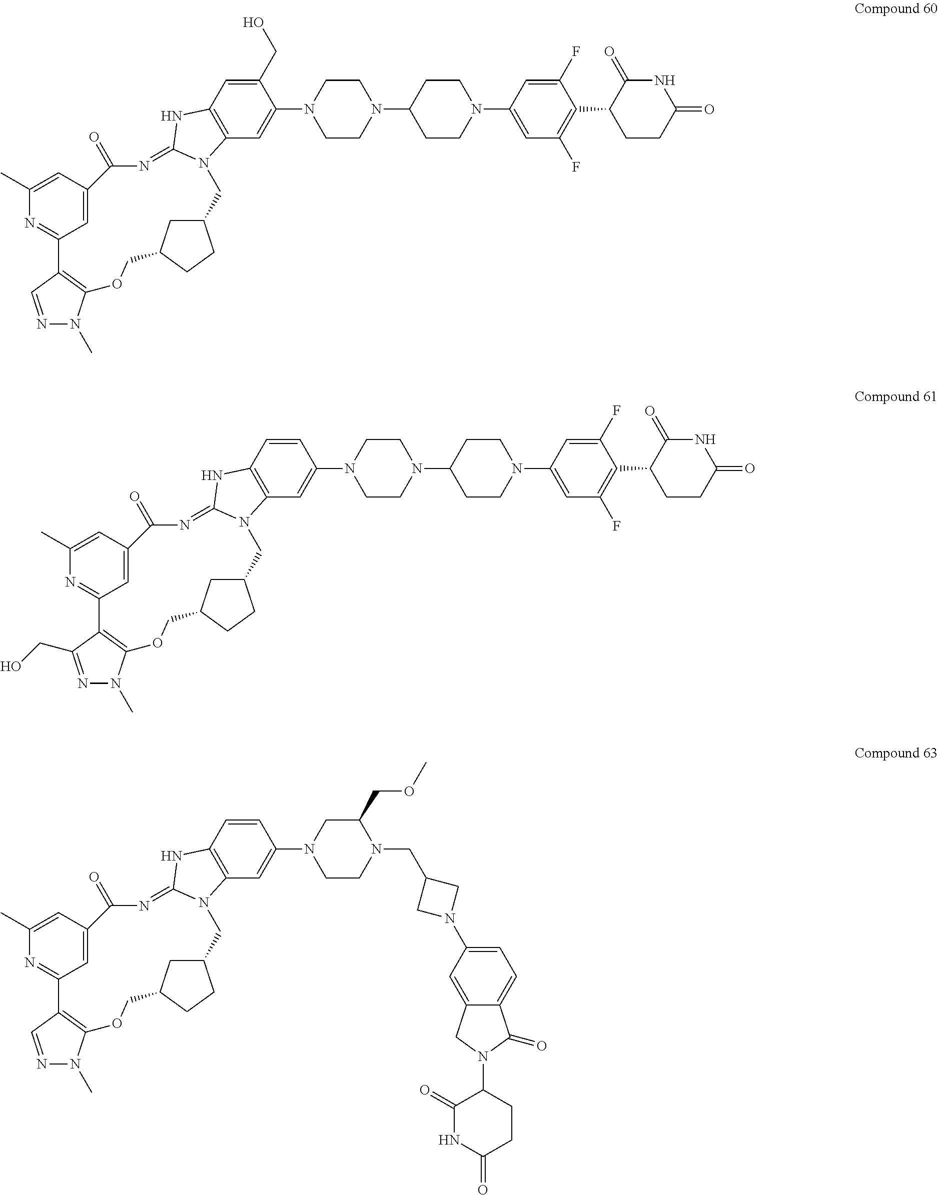 [SMILES: Cc1cc2cc(n1)-c1cnn(C)c1OC[C@H]1CC[C@H](C1)CN1/C(=N/C2=O)Nc2cc(CO)c(N3CCN(C4CCN(c5cc(F)c([C@H]6CCC(=O)NC6=O)c(F)c5)CC4)CC3)cc21 ; Cc1cc2cc(n1)-c1c(CO)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)CC3)cc21 ; COC[C@@H]1CN(c2ccc3c(c2)N2C[C@@H]4CC[C@H](COc5c(cnn5C)-c5cc(cc(C)n5)C(=O)/N=C/2N3)C4)CCN1CC1CN(c2ccc3c(c2)CN(C2CCC(=O)NC2=O)C3=O)C1]

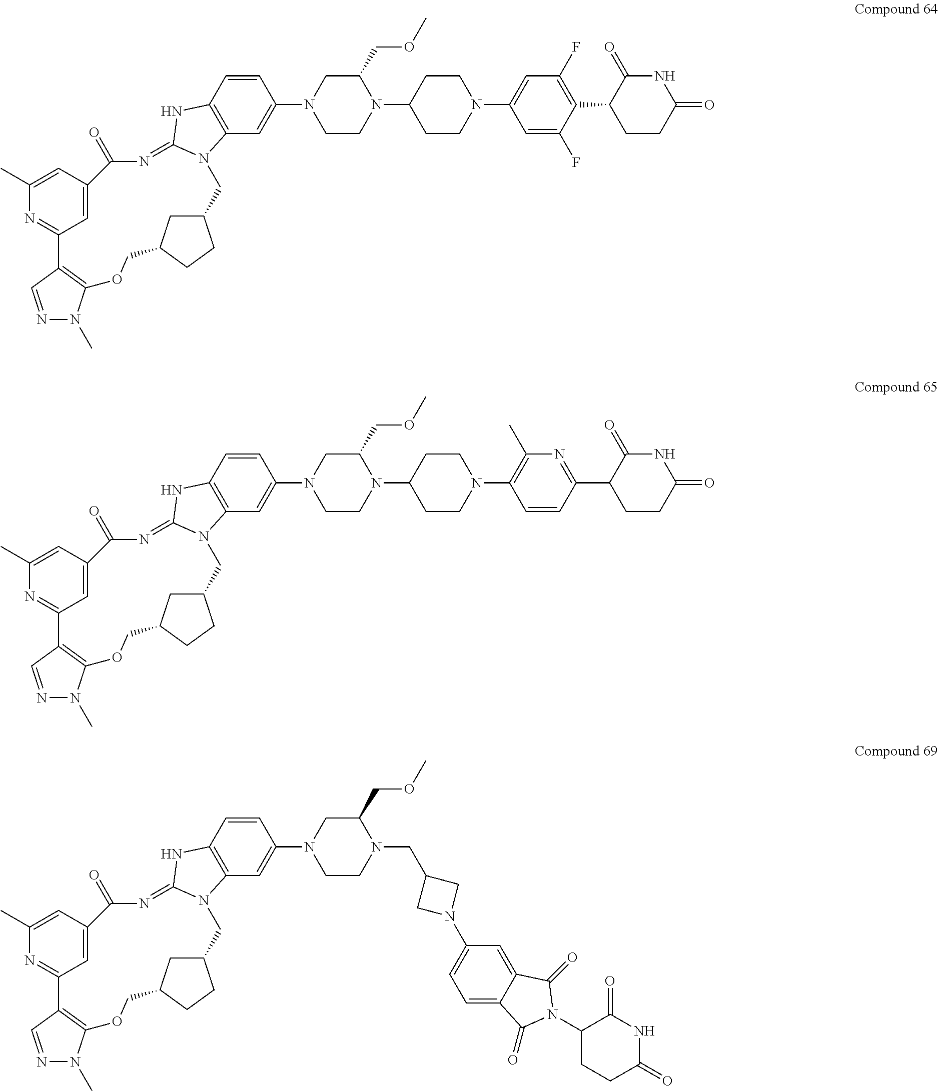 [SMILES: COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1 ; COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2ccc(C3CCC(=O)NC3=O)nc2C)CC1 ; COC[C@@H]1CN(c2ccc3c(c2)N2C[C@@H]4CC[C@H](COc5c(cnn5C)-c5cc(cc(C)n5)C(=O)/N=C/2N3)C4)CCN1CC1CN(c2ccc3c(c2)C(=O)N(C2CCC(=O)NC2=O)C3=O)C1]

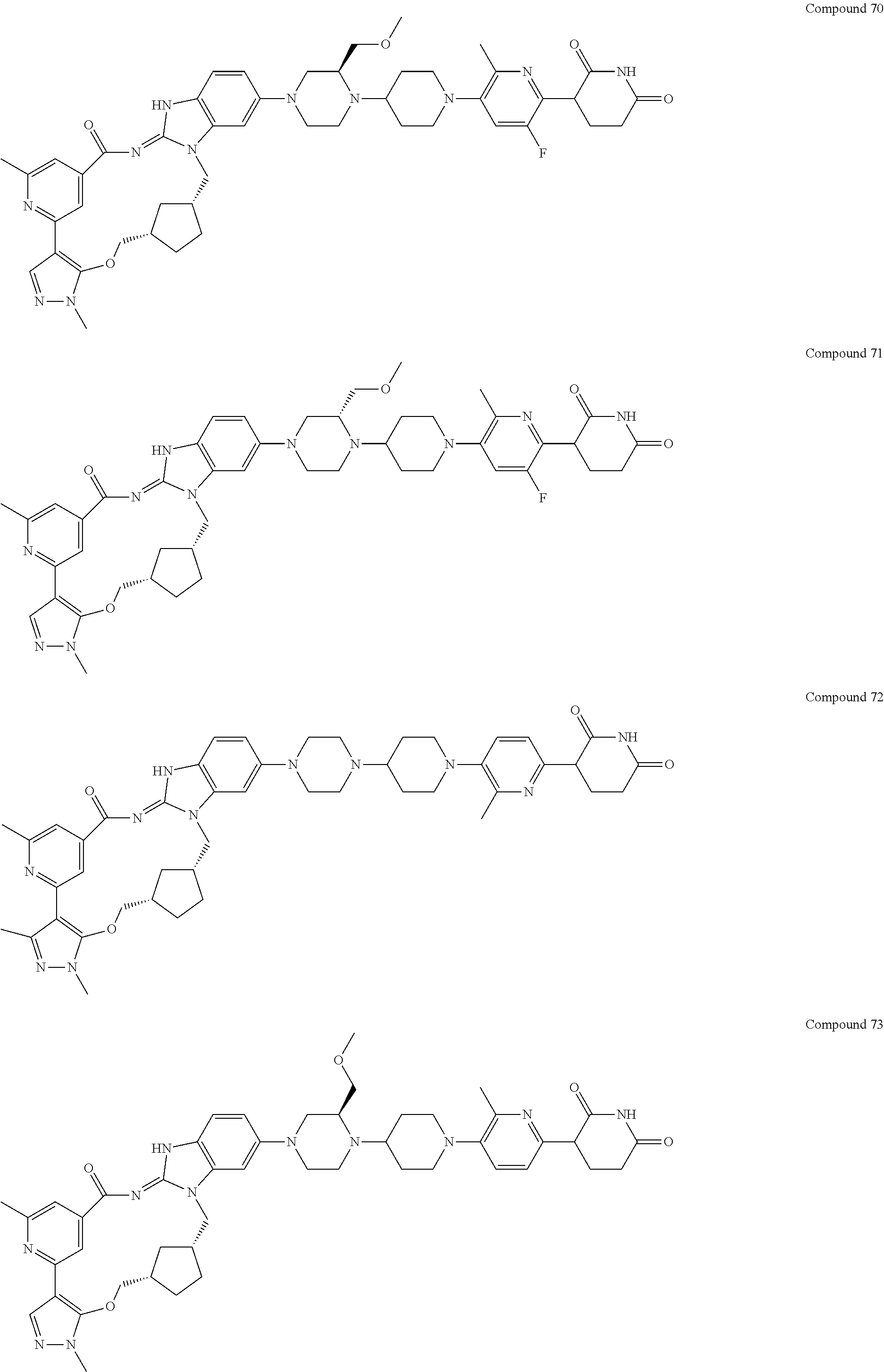

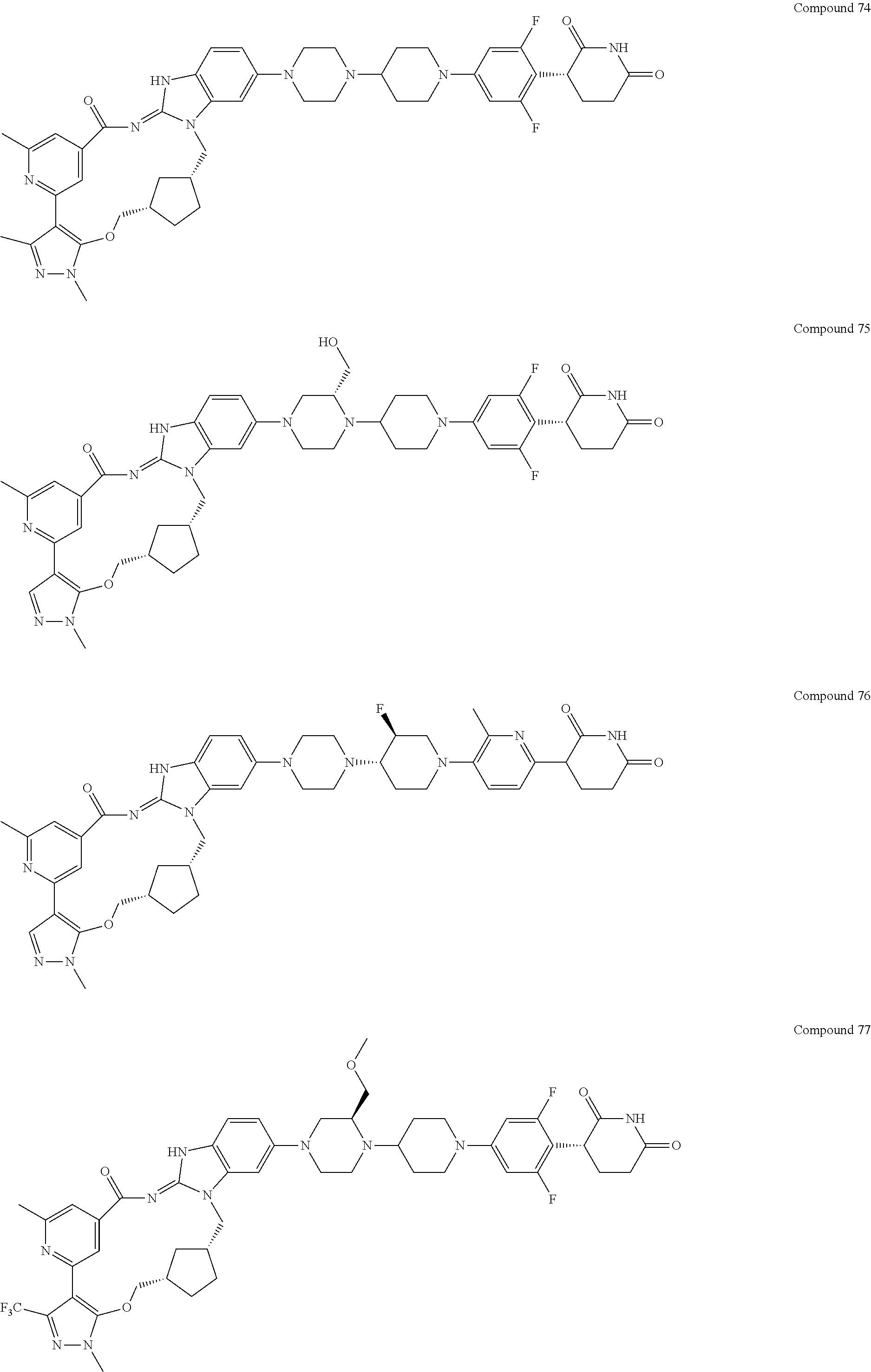

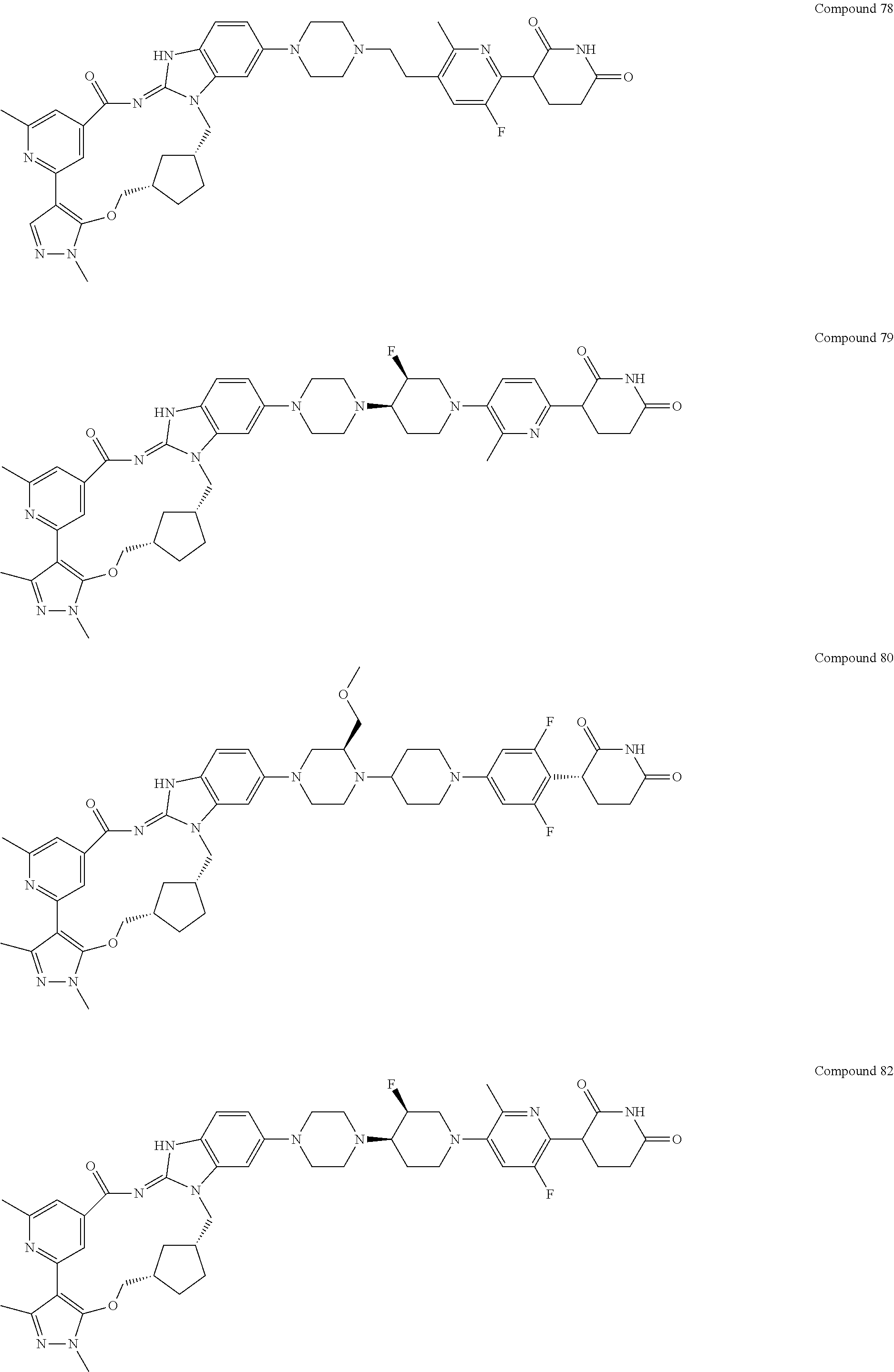

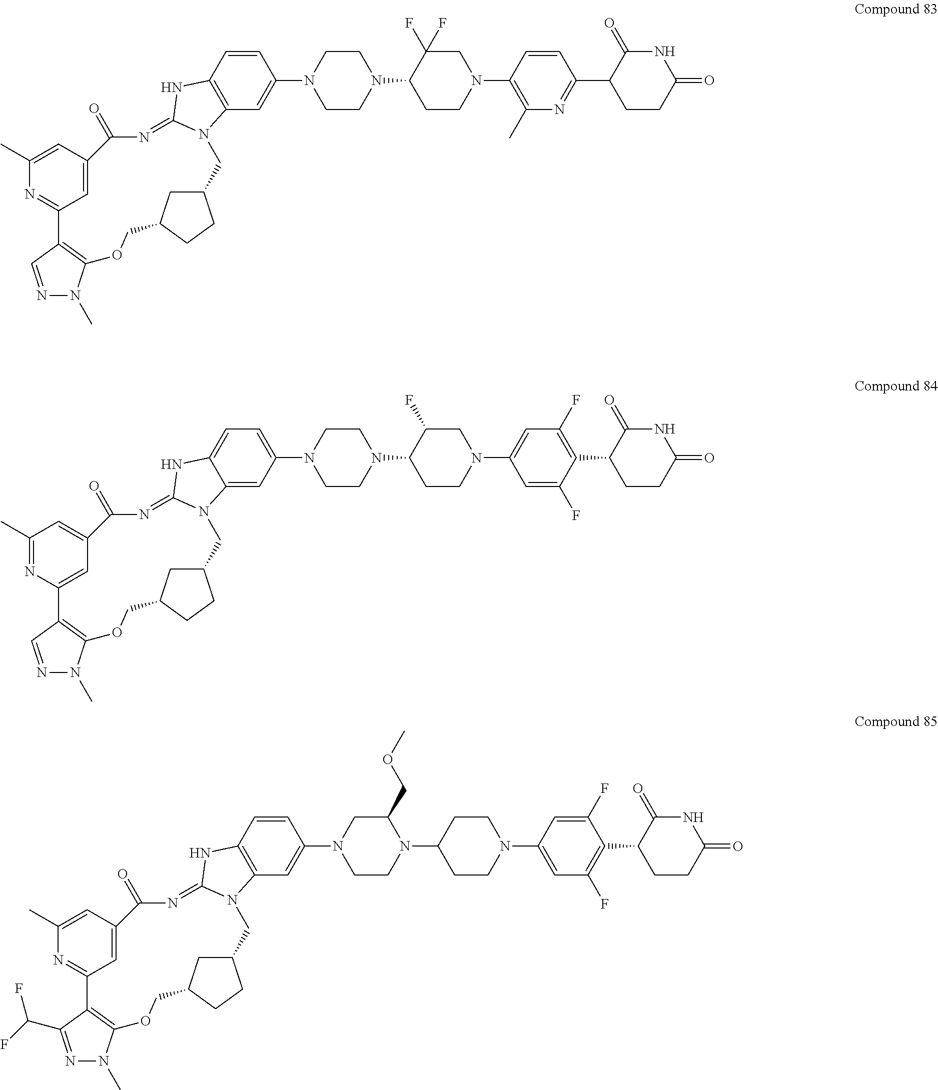 [SMILES: Cc1cc2cc(n1)-c1cnn(C)c1OC[C@H]1CC[C@H](C1)CN1/C(=N/C2=O)Nc2ccc(N3CCN([C@H]4CCN(c5ccc(C6CCC(=O)NC6=O)nc5C)CC4(F)F)CC3)cc21 ; Cc1cc2cc(n1)-c1cnn(C)c1OC[C@H]1CC[C@H](C1)CN1/C(=N/C2=O)Nc2ccc(N3CCN([C@H]4CCN(c5cc(F)c([C@H]6CCC(=O)NC6=O)c(F)c5)C[C@H]4F)CC3)cc21 ; COC[C@@H]1CN(c2ccc3c(c2)N2C[C@@H]4CC[C@H](COc5c(c(C(F)F)nn5C)-c5cc(cc(C)n5)C(=O)/N=C/2N3)C4)CCN1C1CCN(c2cc(F)c([C@H]3CCC(=O)NC3=O)c(F)c2)CC1]

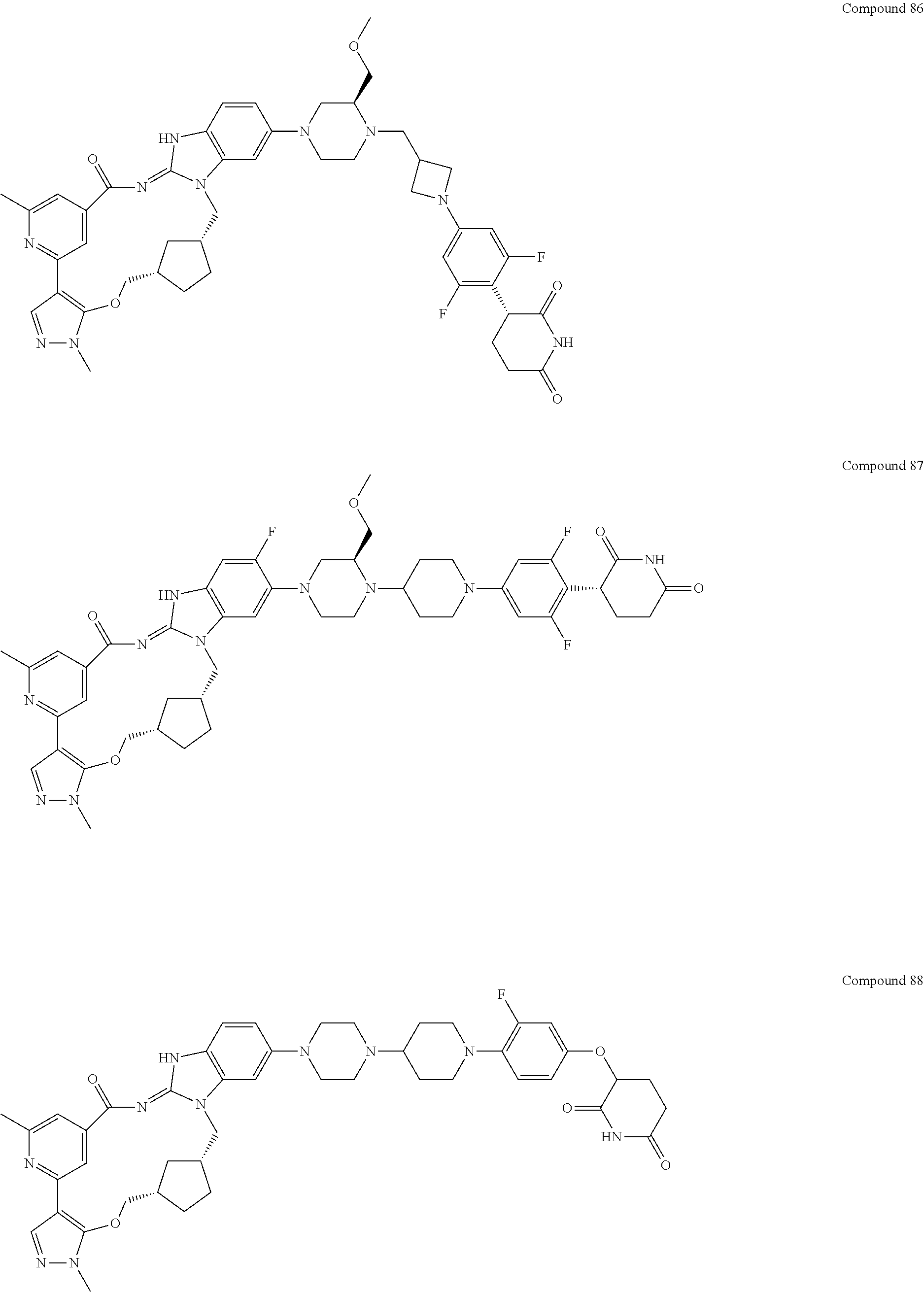 [SMILES: COC[C@@H]1CN(c2ccc3c(c2)N2C[C@@H]4CC[C@H](COc5c(cnn5C)-c5cc(cc(C)n5)C(=O)/N=C/2N3)C4)CCN1CC1CN(c2cc(F)c([C@H]3CCC(=O)NC3=O)c(F)c2)C1 ; COC[C@@H]1CN(c2cc3c(cc2F)N/C2=N\C(=O)c4cc(C)nc(c4)-c4cnn(C)c4OC[C@H]4CC[C@H](C4)CN23)CCN1C1CCN(c2cc(F)c([C@H]3CCC(=O)NC3=O)c(F)c2)CC1 ; Cc1cc2cc(n1)-c1cnn(C)c1OC[C@H]1CC[C@H](C1)CN1/C(=N/C2=O)Nc2ccc(N3CCN(C4CCN(c5ccc(OC6CCC(=O)NC6=O)cc5F)CC4)CC3)cc21]

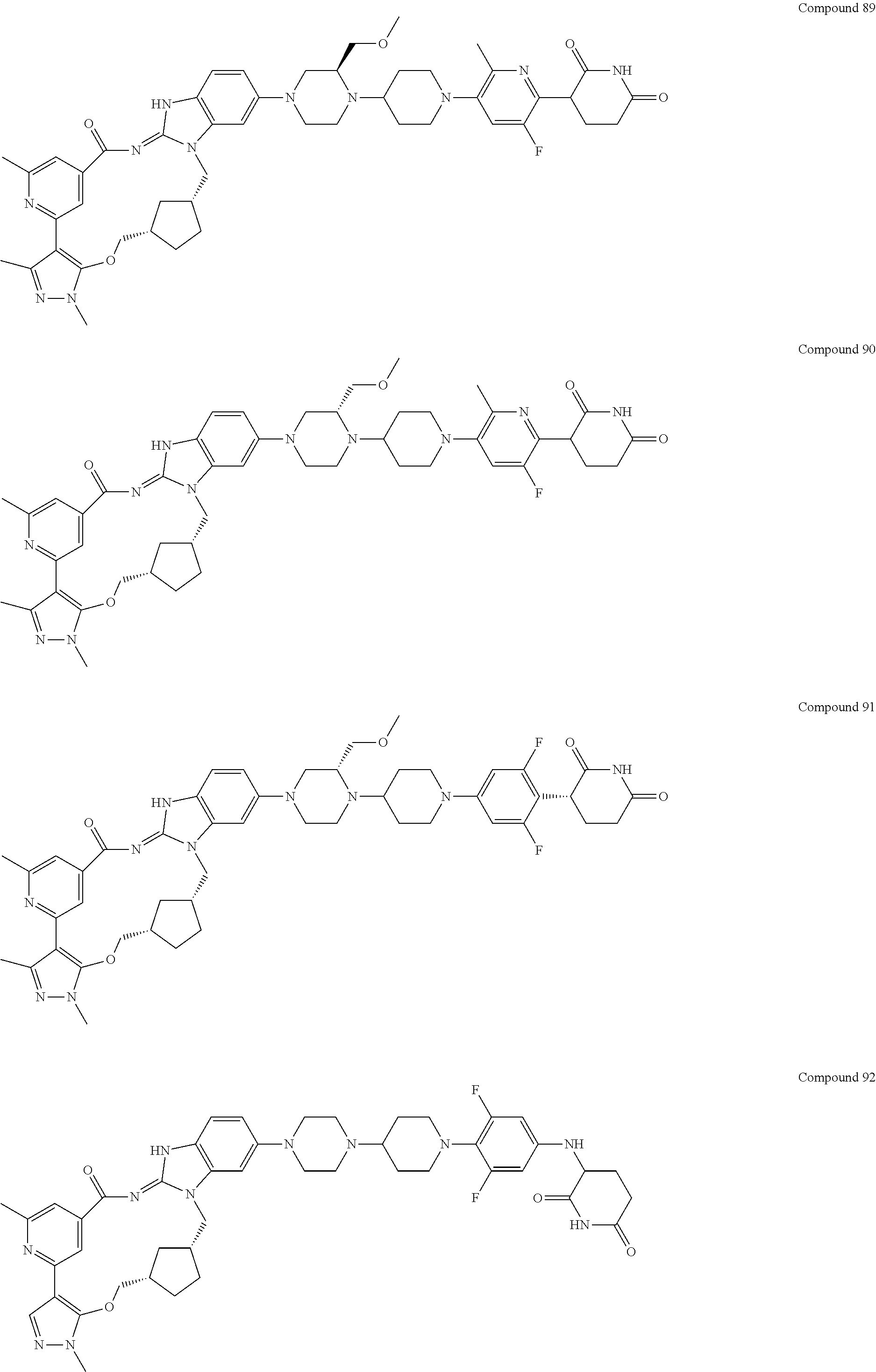

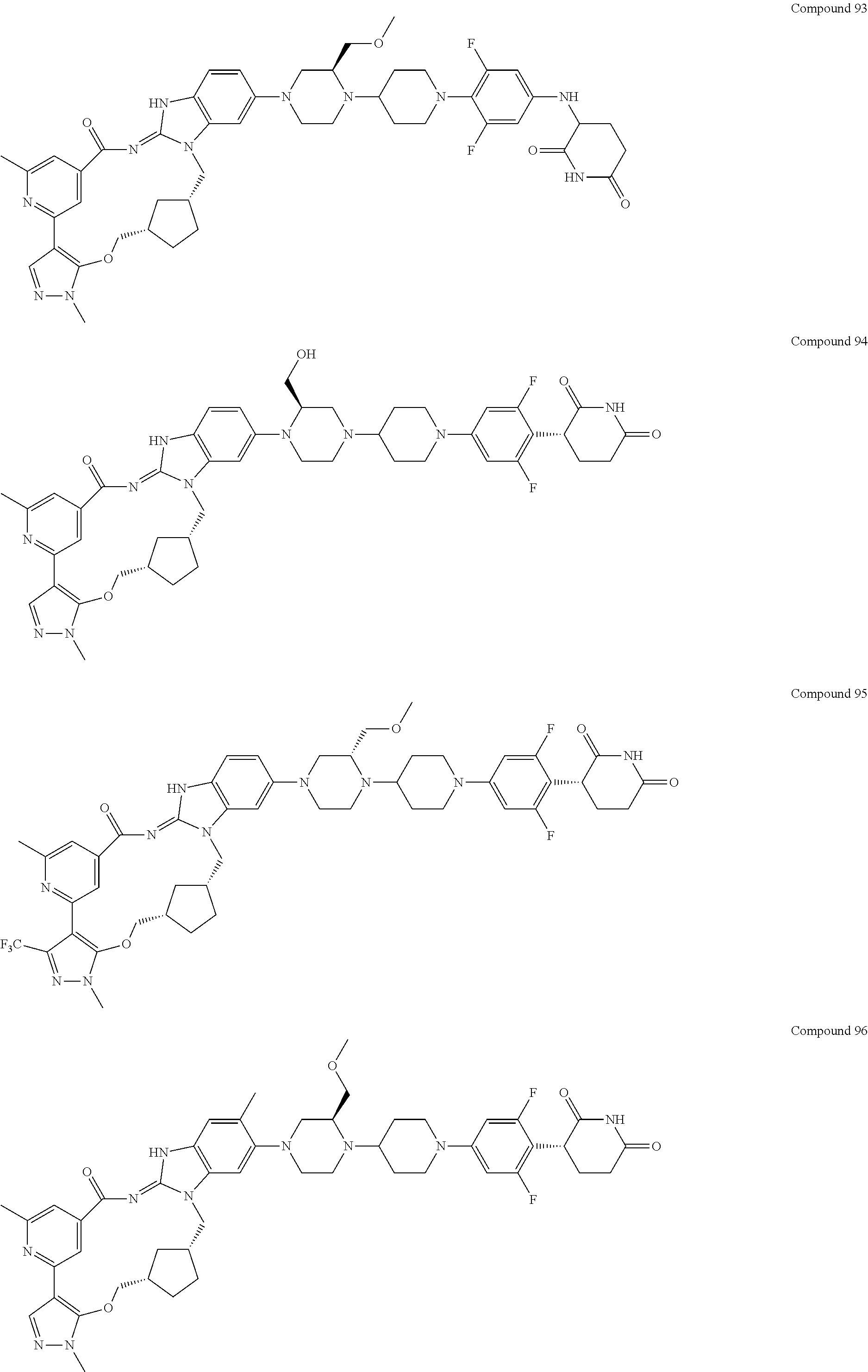

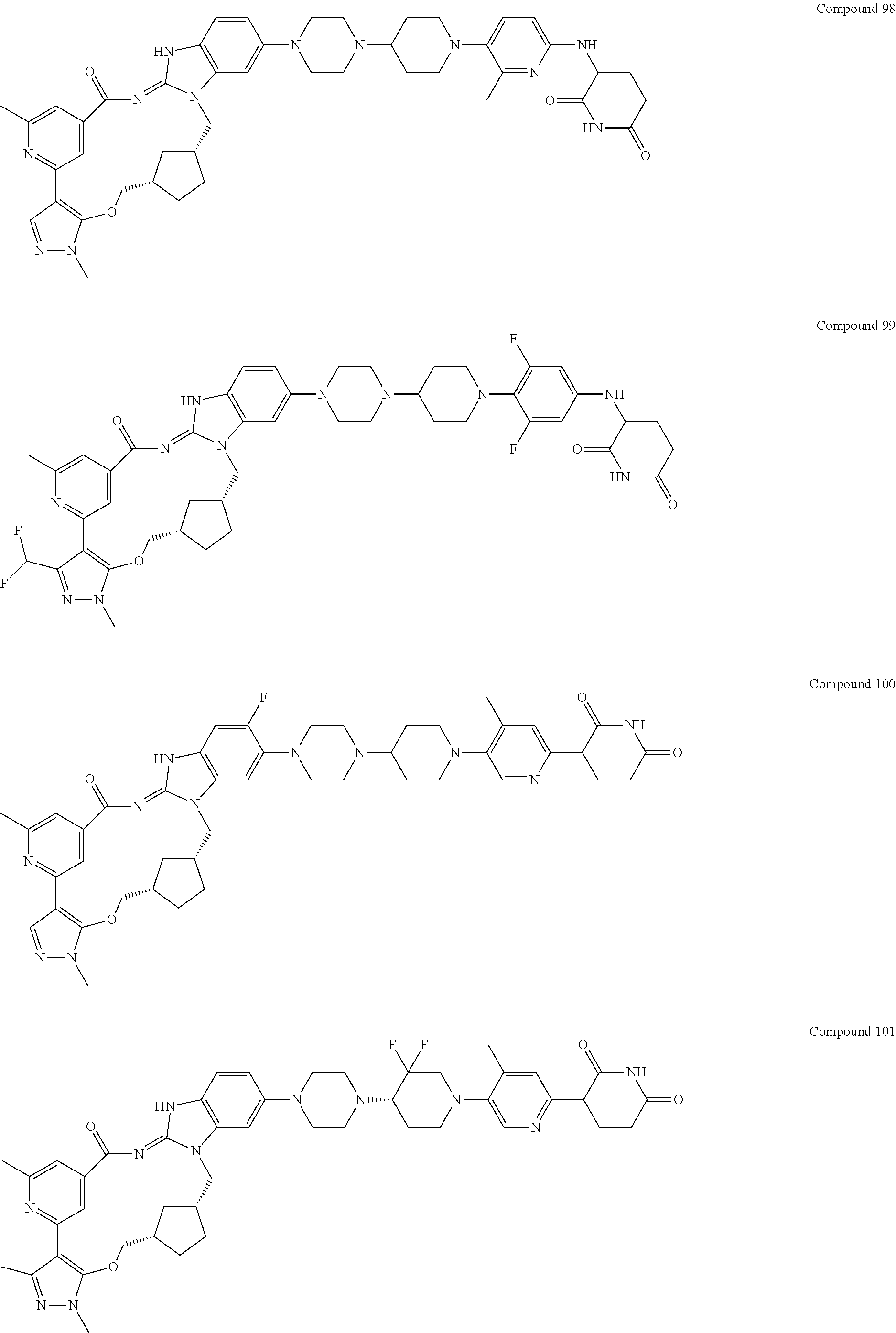 [SMILES: Cc1cc2cc(n1)-c1cnn(C)c1OC[C@H]1CC[C@H](C1)CN1/C(=N/C2=O)Nc2ccc(N3CCN(C4CCN(c5ccc(NC6CCC(=O)NC6=O)nc5C)CC4)CC3)cc21 ; Cc1cc2cc(n1)-c1c(C(F)F)nn(C)c1OC[C@H]1CC[C@H](C1)CN1/C(=N/C2=O)Nc2ccc(N3CCN(C4CCN(c5c(F)cc(NC6CCC(=O)NC6=O)cc5F)CC4)CC3)cc21 ; Cc1cc2cc(n1)-c1cnn(C)c1OC[C@H]1CC[C@H](C1)CN1/C(=N/C2=O)Nc2cc(F)c(N3CCN(C4CCN(c5cnc(C6CCC(=O)NC6=O)cc5C)CC4)CC3)cc21 ; Cc1cc2cc(n1)-c1c(C)nn(C)c1OC[C@H]1CC[C@H](C1)CN1/C(=N/C2=O)Nc2ccc(N3CCN([C@H]4CCN(c5cnc(C6CCC(=O)NC6=O)cc5C)CC4(F)F)CC3)cc21]

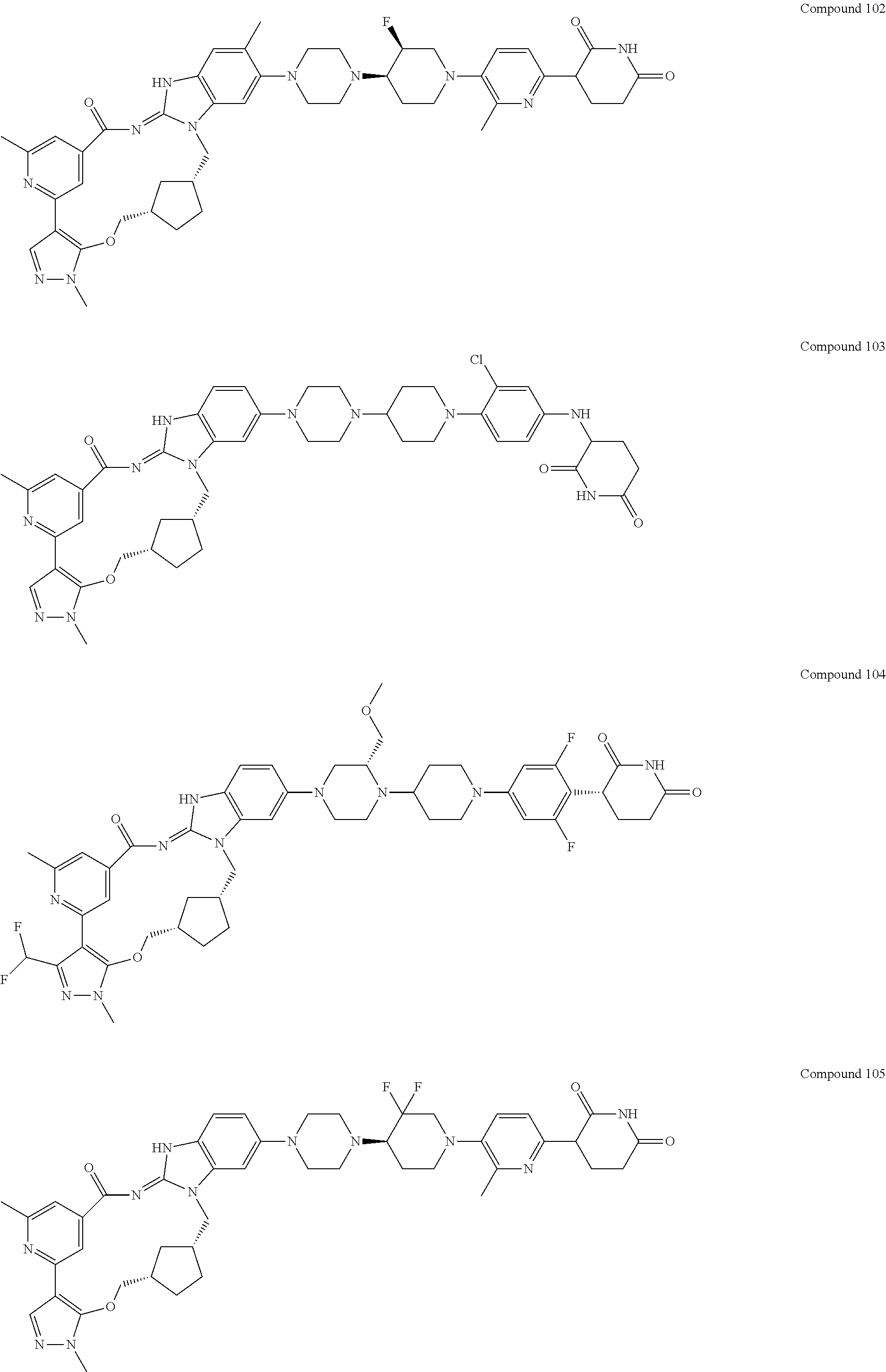

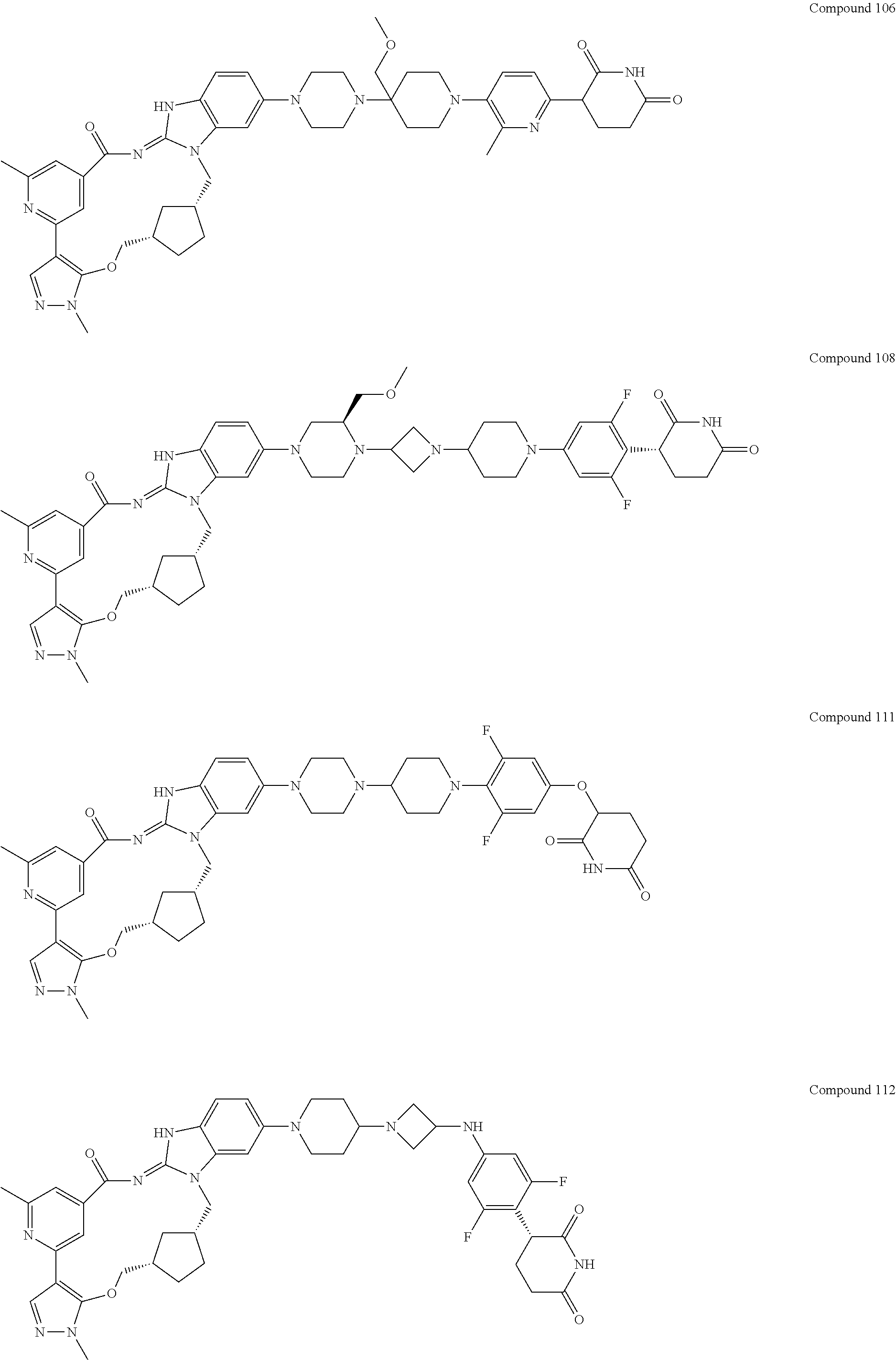

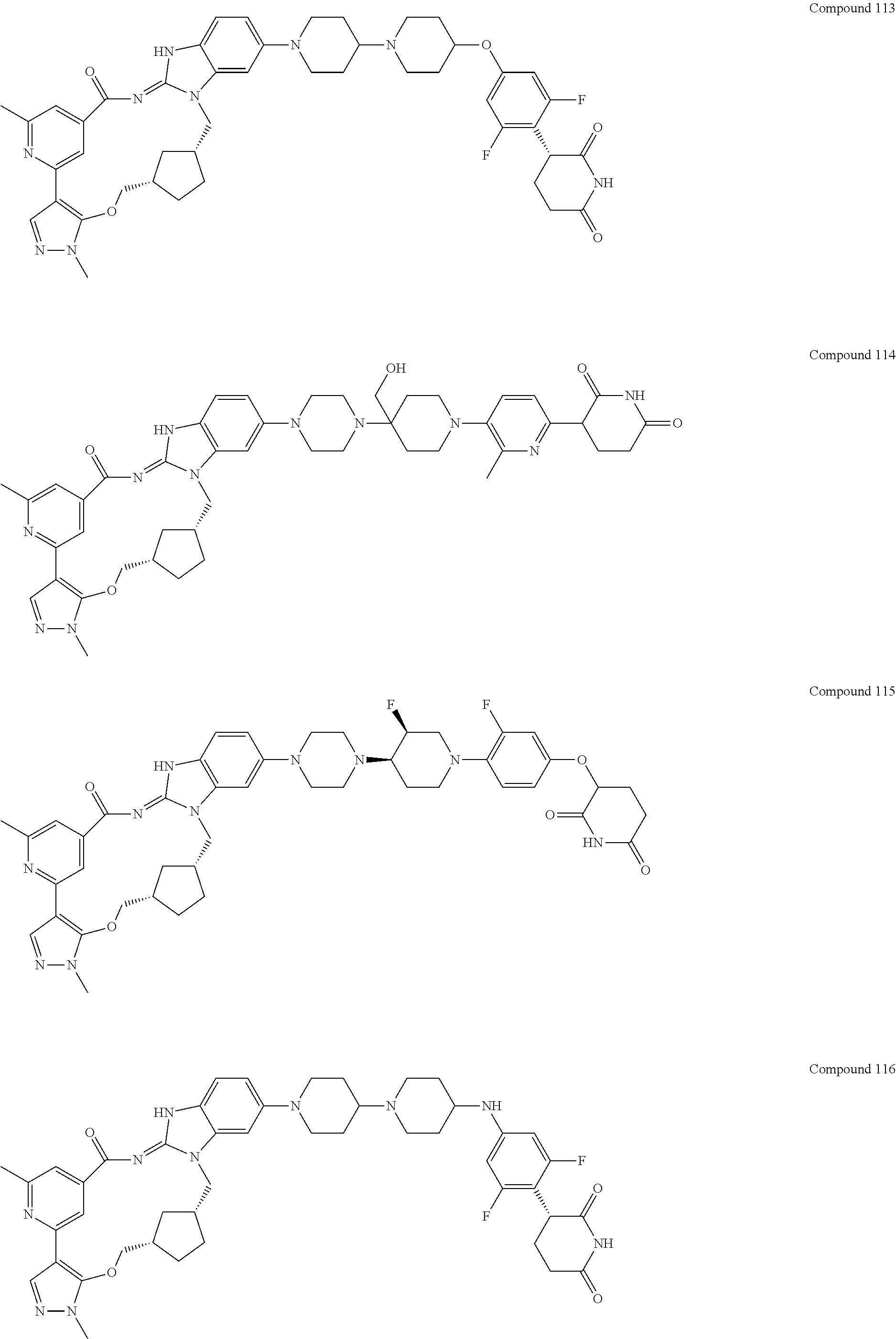 [SMILES: Cc1cc2cc(n1)-c1cnn(C)c1OC[C@H]1CC[C@H](C1)CN1/C(=N/C2=O)Nc2ccc(N3CCC(N4CCC(Oc5cc(F)c([C@H]6CCC(=O)NC6=O)c(F)c5)CC4)CC3)cc21 ; Cc1cc2cc(n1)-c1cnn(C)c1OC[C@H]1CC[C@H](C1)CN1/C(=N/C2=O)Nc2ccc(N3CCN(C4(CO)CCN(c5ccc(C6CCC(=O)NC6=O)nc5C)CC4)CC3)cc21 ; Cc1cc2cc(n1)-c1cnn(C)c1OC[C@H]1CC[C@H](C1)CN1/C(=N/C2=O)Nc2ccc(N3CCN([C@@H]4CCN(c5ccc(OC6CCC(=O)NC6=O)cc5F)C[C@@H]4F)CC3)cc21 ; Cc1cc2cc(n1)-c1cnn(C)c1OC[C@H]1CC[C@H](C1)CN1/C(=N/C2=O)Nc2ccc(N3CCC(N4CCC(Nc5cc(F)c([C@H]6CCC(=O)NC6=O)c(F)c5)CC4)CC3)cc21]

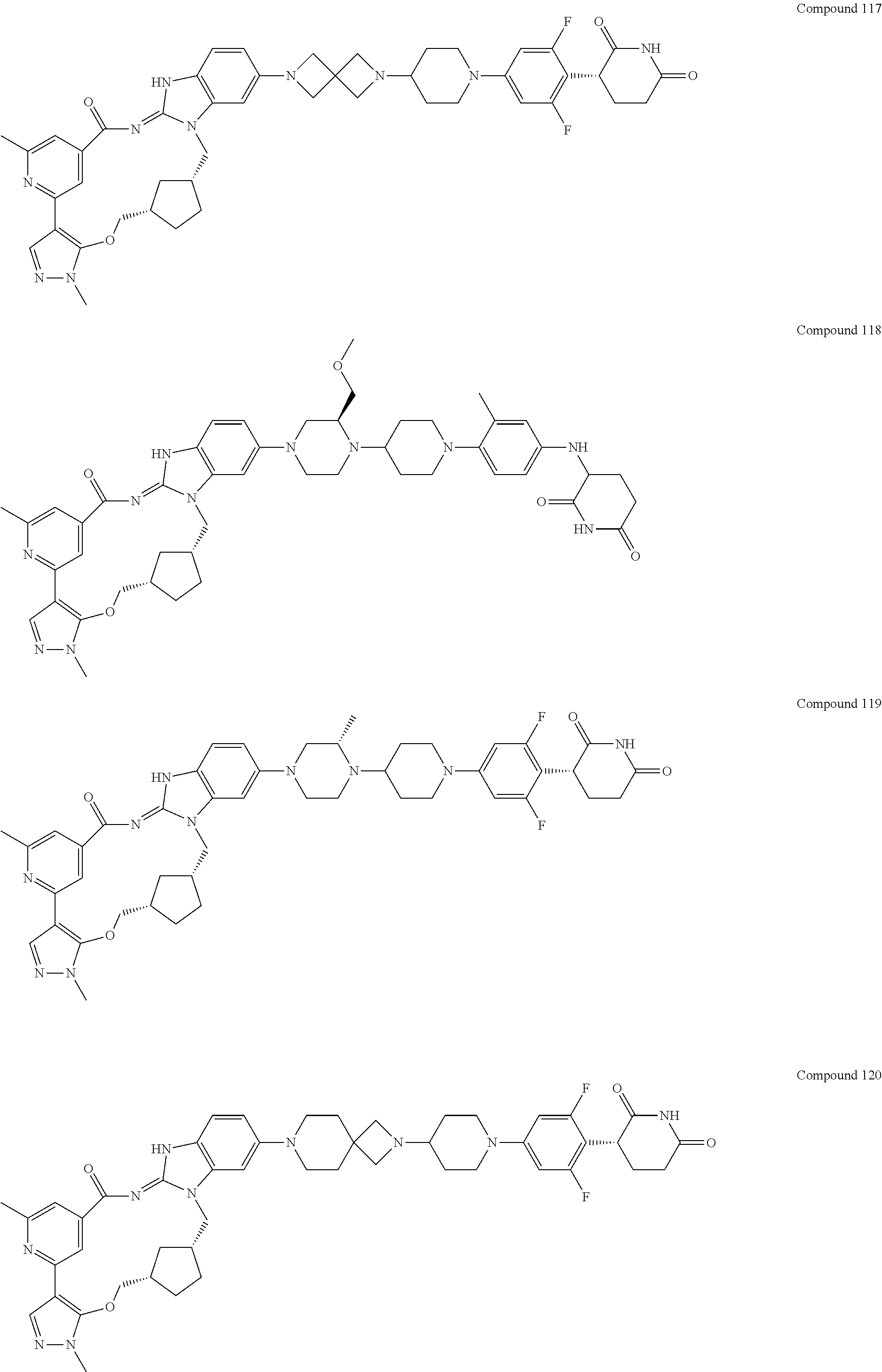

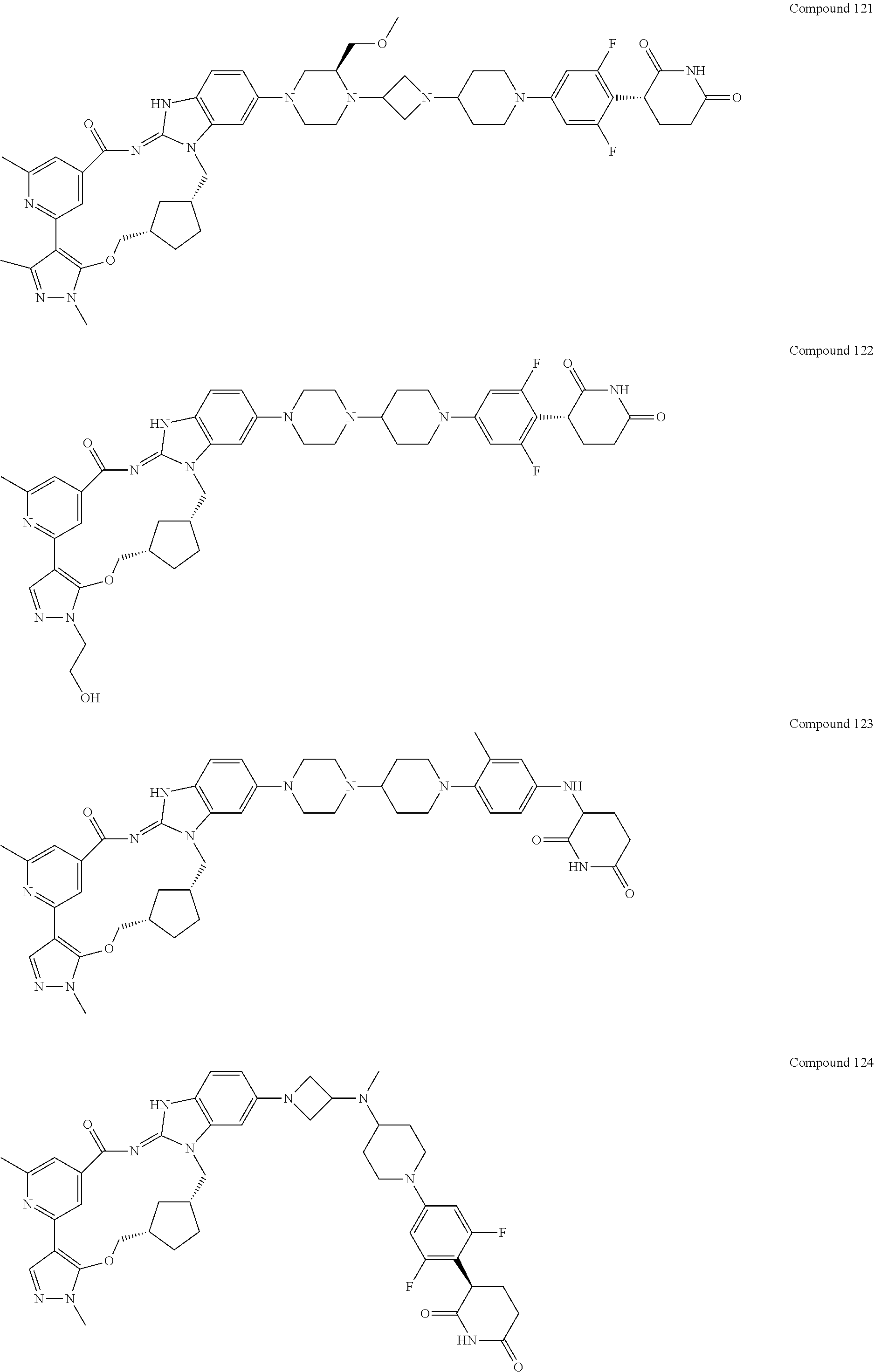

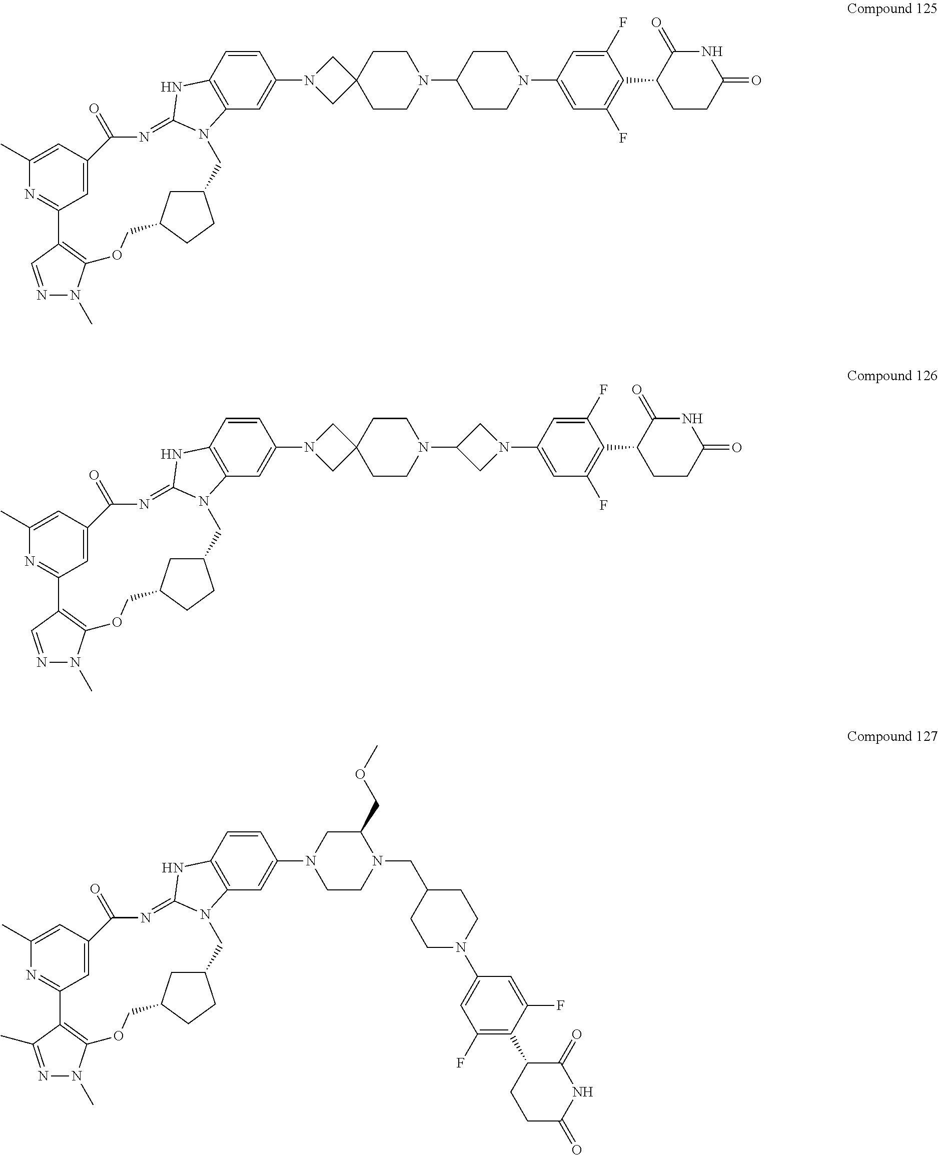 [SMILES: Cc1cc2cc(n1)-c1cnn(C)c1OC[C@H]1CC[C@H](C1)CN1/C(=N/C2=O)Nc2ccc(N3CC4(CCN(C5CCN(c6cc(F)c([C@H]7CCC(=O)NC7=O)c(F)c6)CC5)CC4)C3)cc21 ; Cc1cc2cc(n1)-c1cnn(C)c1OC[C@H]1CC[C@H](C1)CN1/C(=N/C2=O)Nc2ccc(N3CC4(CCN(C5CN(c6cc(F)c([C@H]7CCC(=O)NC7=O)c(F)c6)C5)CC4)C3)cc21 ; COC[C@@H]1CN(c2ccc3c(c2)N2C[C@@H]4CC[C@H](COc5c(c(C)nn5C)-c5cc(cc(C)n5)C(=O)/N=C/2N3)C4)CCN1CC1CCN(c2cc(F)c([C@H]3CCC(=O)NC3=O)c(F)c2)CC1]

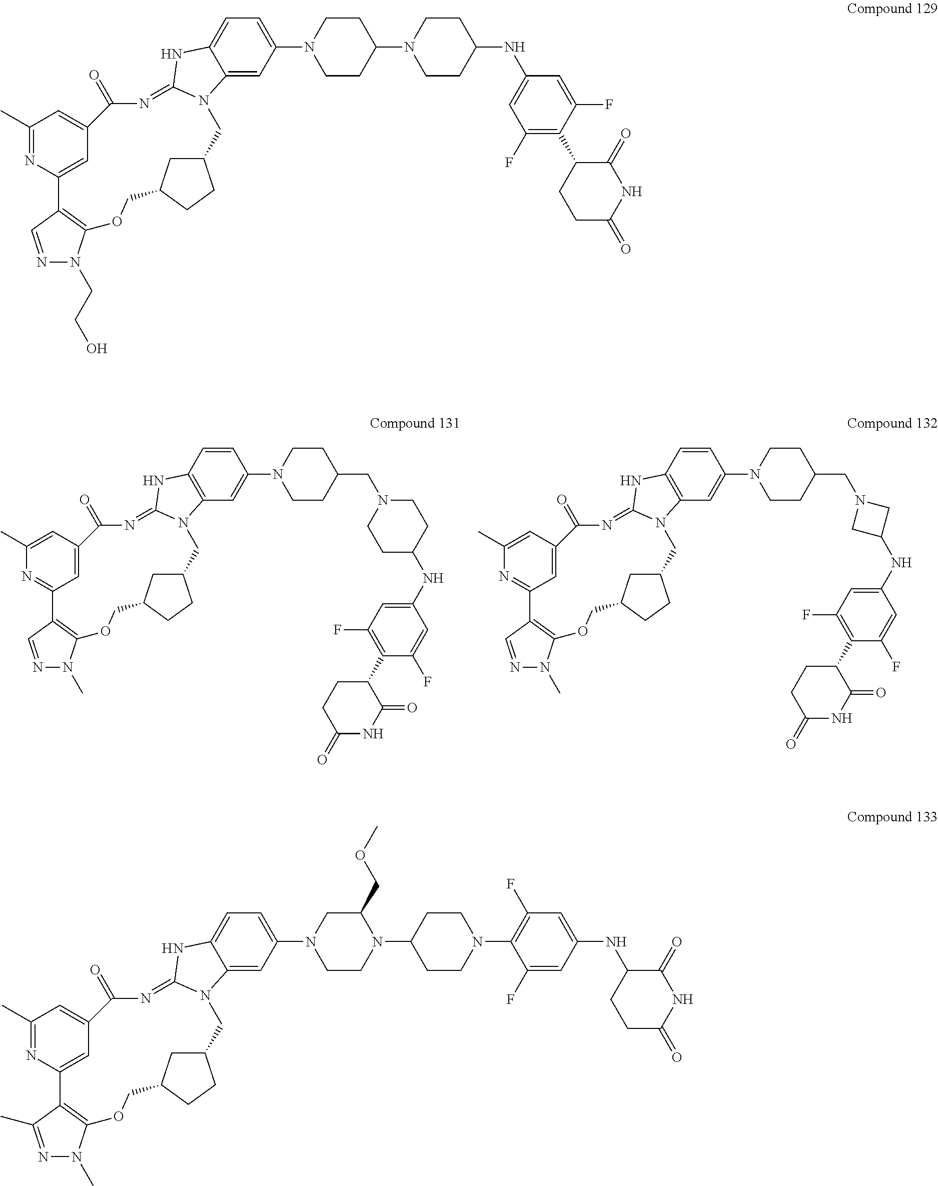

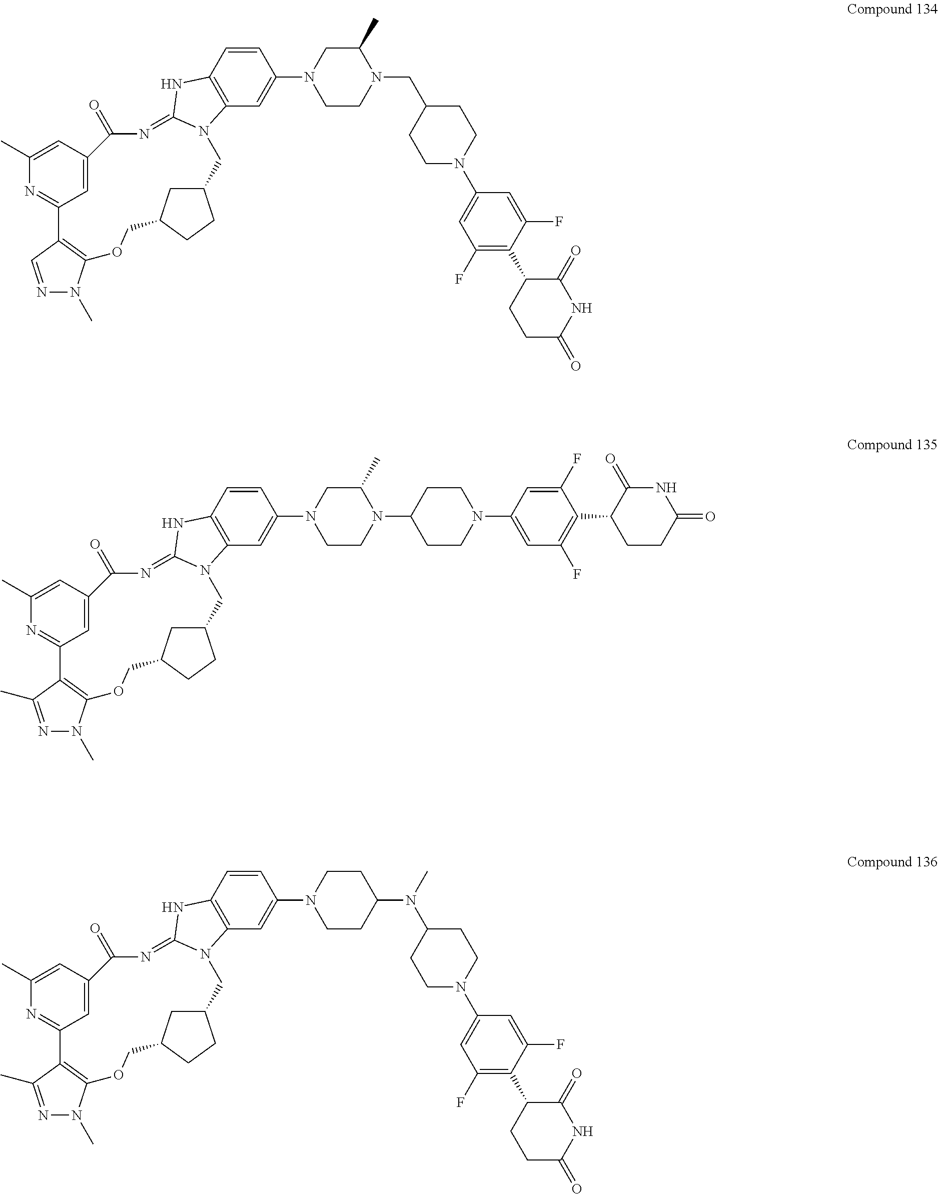 [SMILES: Cc1cc2cc(n1)-c1cnn(C)c1OC[C@H]1CC[C@H](C1)CN1/C(=N/C2=O)Nc2ccc(N3CCN(CC4CCN(c5cc(F)c([C@H]6CCC(=O)NC6=O)c(F)c5)CC4)[C@H](C)C3)cc21 ; Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)[C@@H](C)C3)cc21 ; Cc1cc2cc(n1)-c1c(C)nn(C)c1OC[C@H]1CC[C@H](C1)CN1/C(=N/C2=O)Nc2ccc(N3CCC(N(C)C4CCN(c5cc(F)c([C@H]6CCC(=O)NC6=O)c(F)c5)CC4)CC3)cc21]

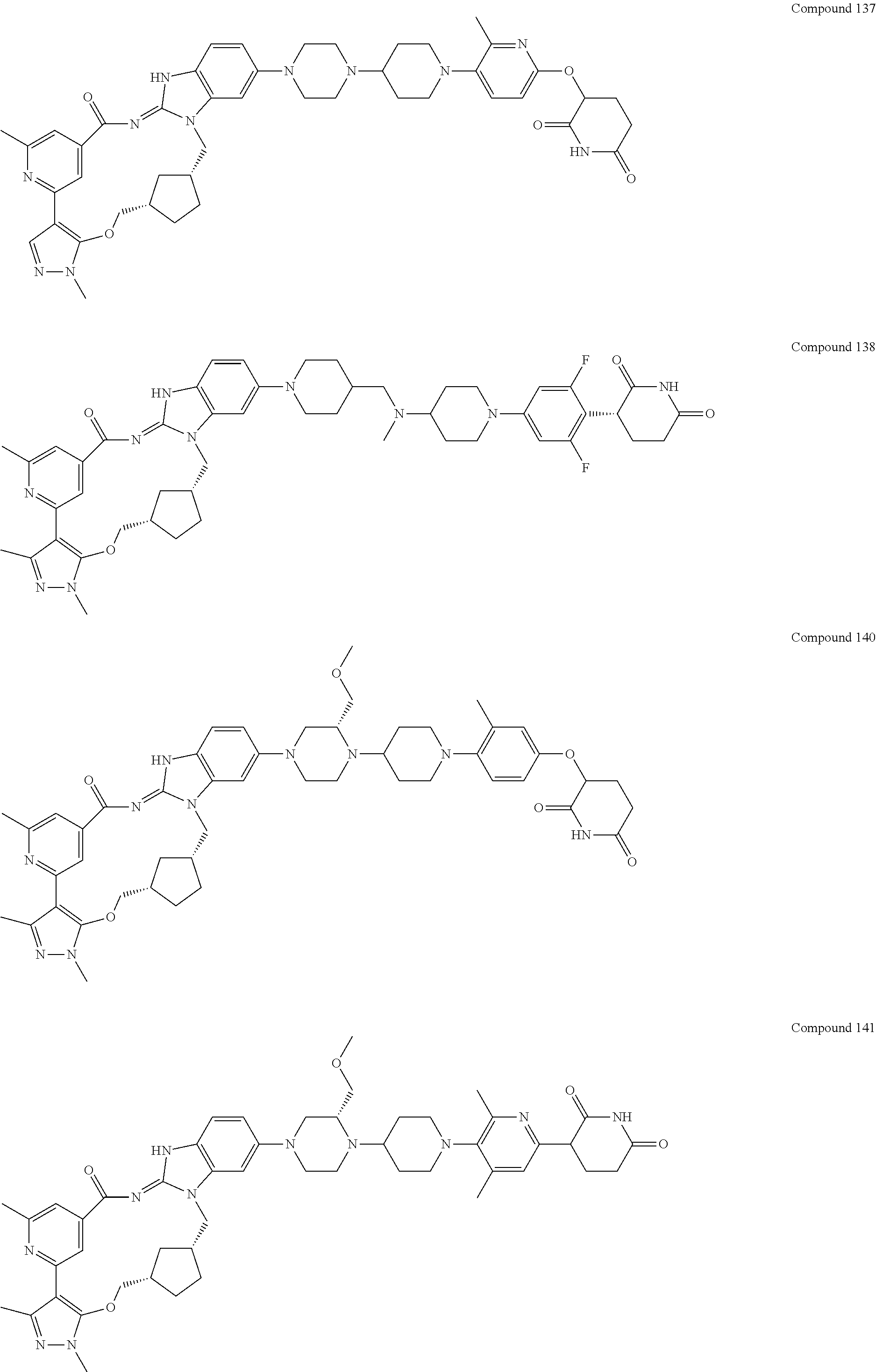

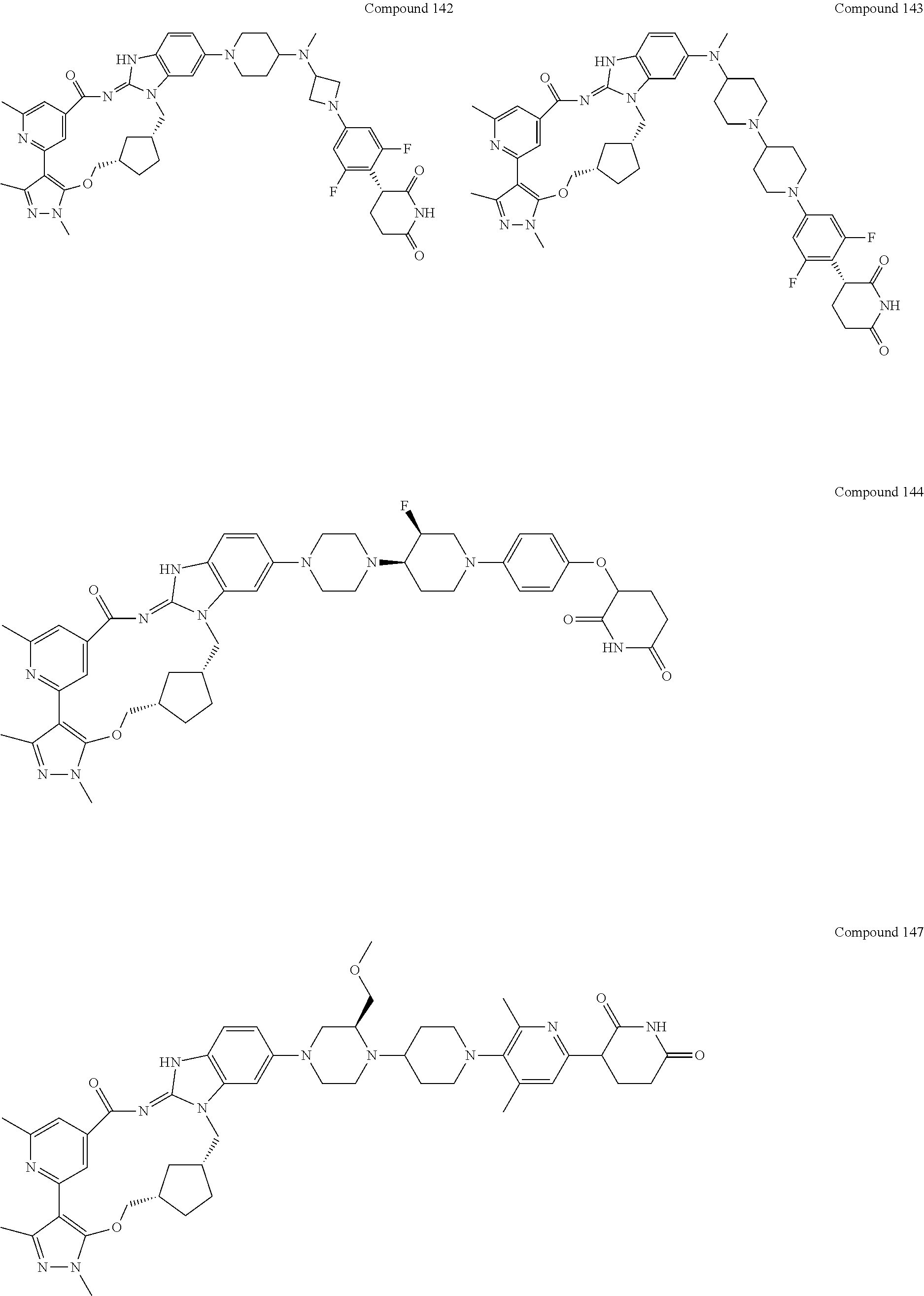

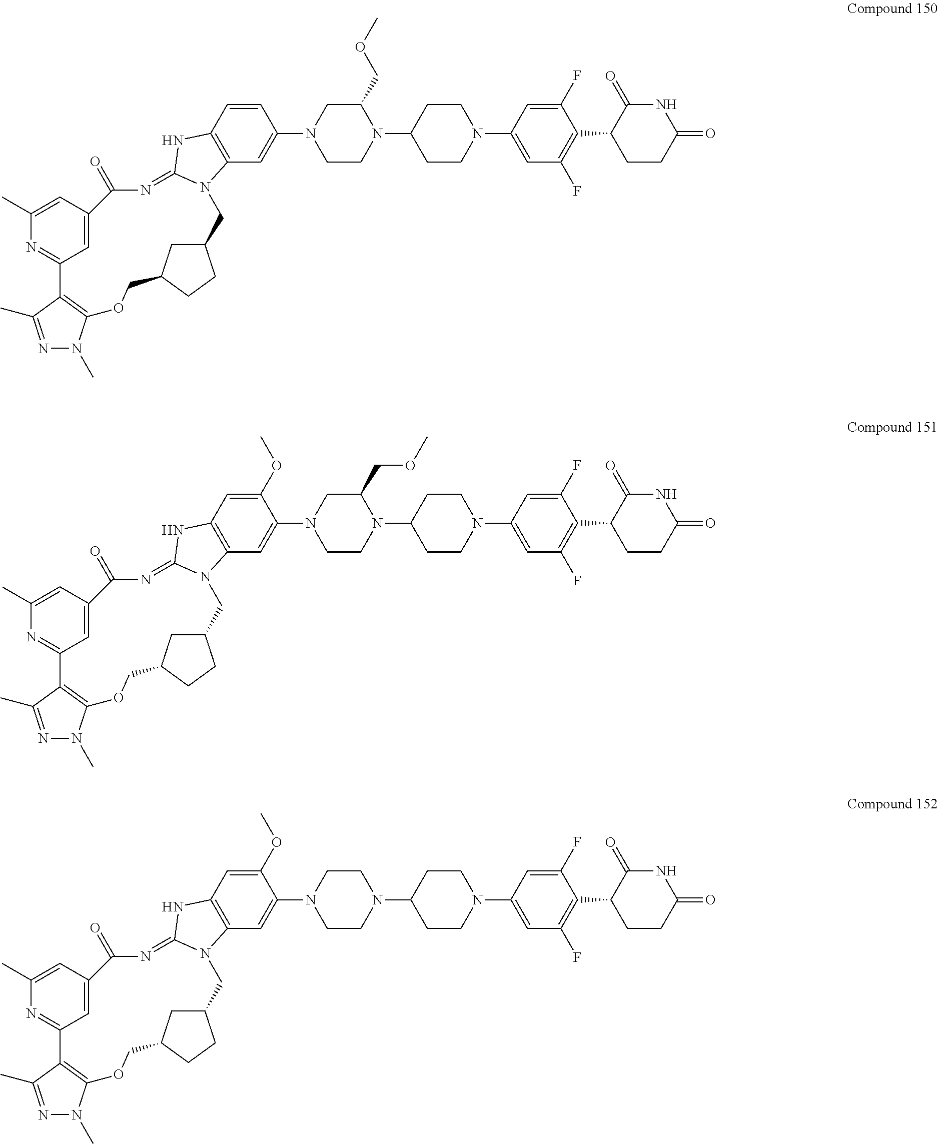 [SMILES: COC[C@H]1CN(c2ccc3c(c2)N2C[C@H]4CC[C@@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1 ; COC[C@@H]1CN(c2cc3c(cc2OC)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1 ; COc1cc2c(cc1N1CCN(C3CCN(c4cc(F)c([C@H]5CCC(=O)NC5=O)c(F)c4)CC3)CC1)N1C[C@@H]3CC[C@H](COc4c(c(C)nn4C)-c4cc(cc(C)n4)C(=O)/N=C/1N2)C3]

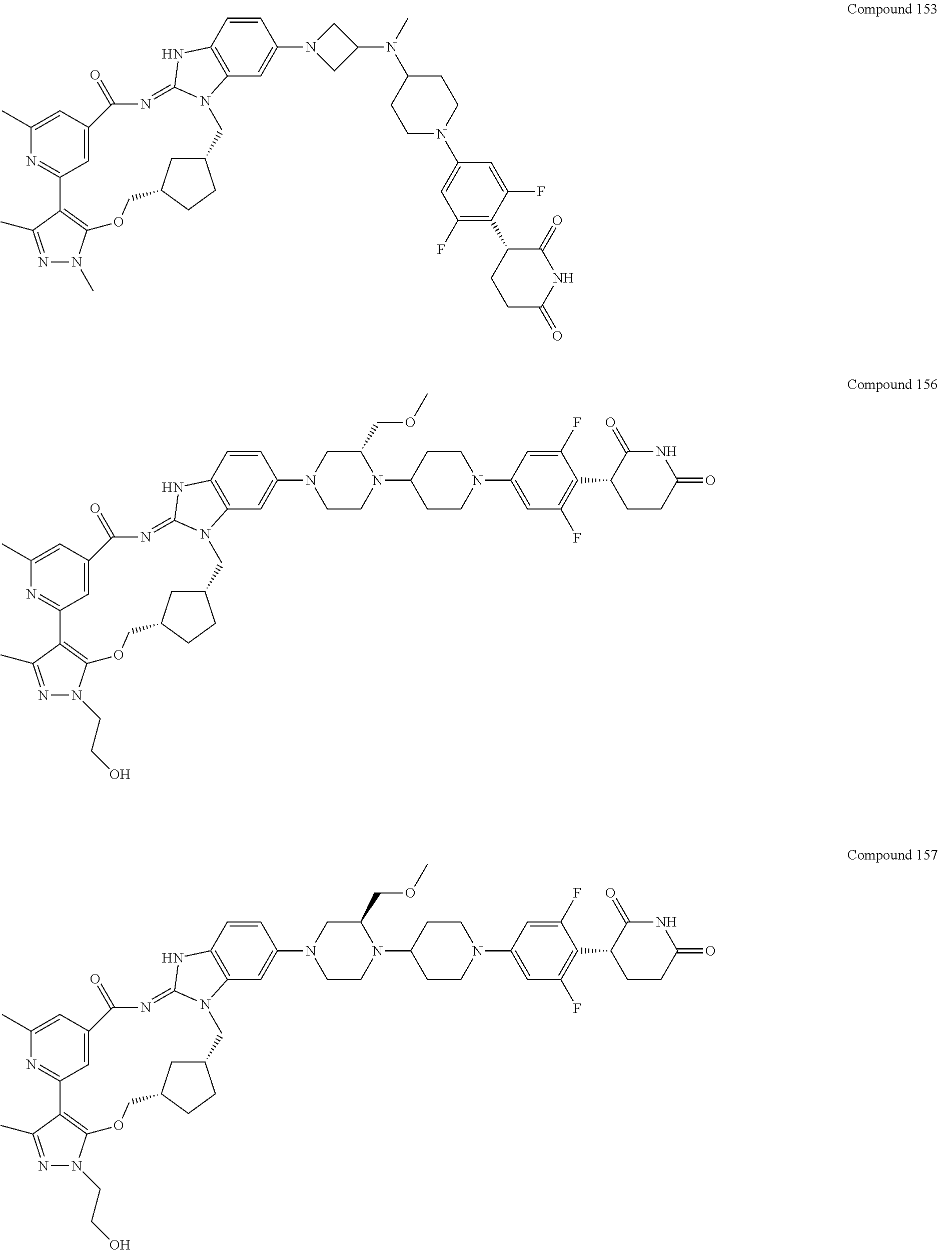 [SMILES: Cc1cc2cc(n1)-c1c(C)nn(C)c1OC[C@H]1CC[C@H](C1)CN1/C(=N/C2=O)Nc2ccc(N3CC(N(C)C4CCN(c5cc(F)c([C@H]6CCC(=O)NC6=O)c(F)c5)CC4)C3)cc21 ; COC[C@H]1CN(c2ccc3c(c2)N2C[C@@H]4CC[C@H](COc5c(c(C)nn5CCO)-c5cc(cc(C)n5)C(=O)/N=C/2N3)C4)CCN1C1CCN(c2cc(F)c([C@H]3CCC(=O)NC3=O)c(F)c2)CC1 ; COC[C@@H]1CN(c2ccc3c(c2)N2C[C@@H]4CC[C@H](COc5c(c(C)nn5CCO)-c5cc(cc(C)n5)C(=O)/N=C/2N3)C4)CCN1C1CCN(c2cc(F)c([C@H]3CCC(=O)NC3=O)c(F)c2)CC1]

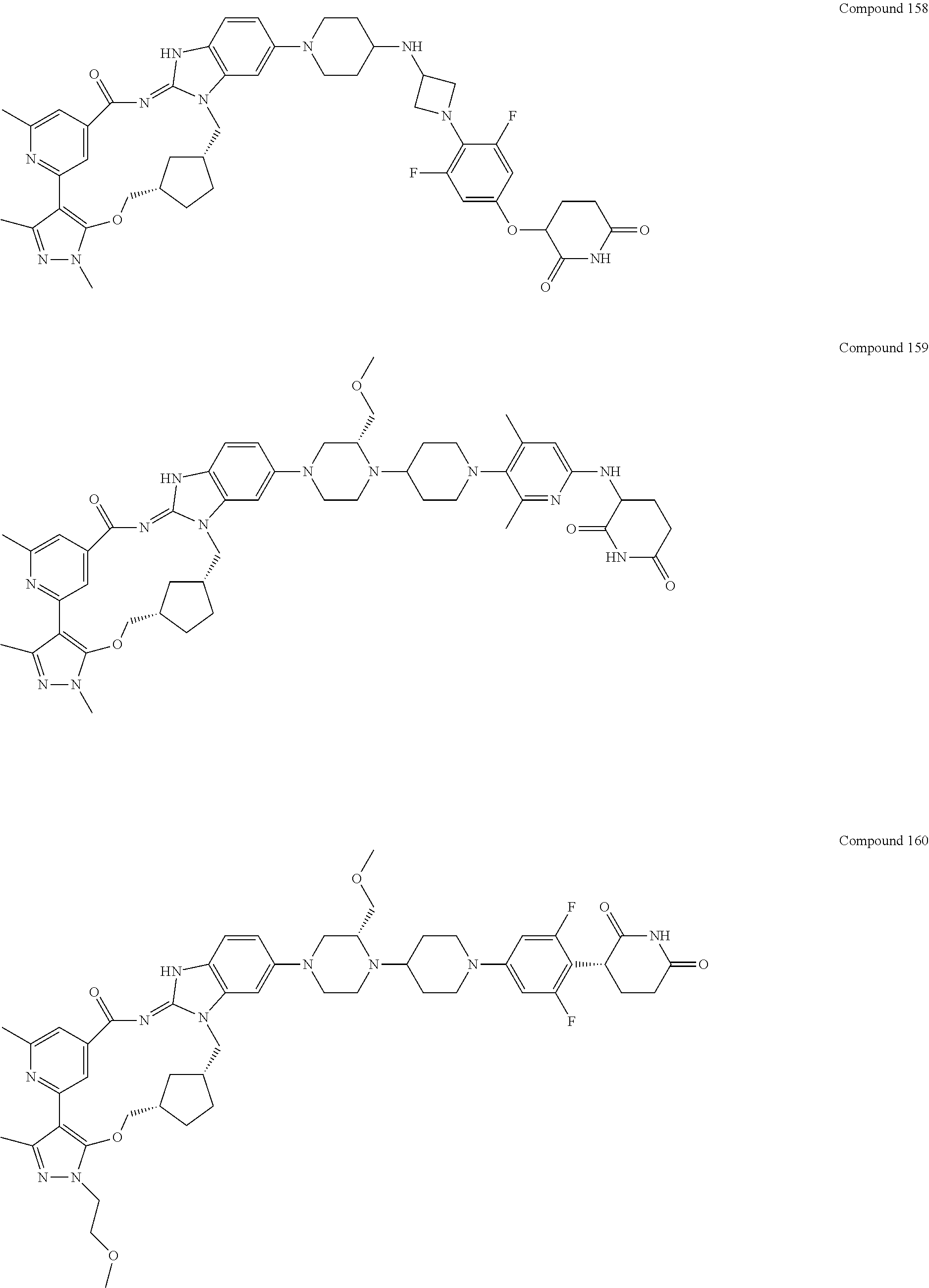 [SMILES: Cc1cc2cc(n1)-c1c(C)nn(C)c1OC[C@H]1CC[C@H](C1)CN1/C(=N/C2=O)Nc2ccc(N3CCC(NC4CN(c5c(F)cc(OC6CCC(=O)NC6=O)cc5F)C4)CC3)cc21 ; COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(NC3CCC(=O)NC3=O)nc2C)CC1 ; COCCn1nc(C)c2c1OC[C@H]1CC[C@H](C1)CN1/C(=N/C(=O)c3cc(C)nc-2c3)Nc2ccc(N3CCN(C4CCN(c5cc(F)c([C@H]6CCC(=O)NC6=O)c(F)c5)CC4)[C@@H](COC)C3)cc21]

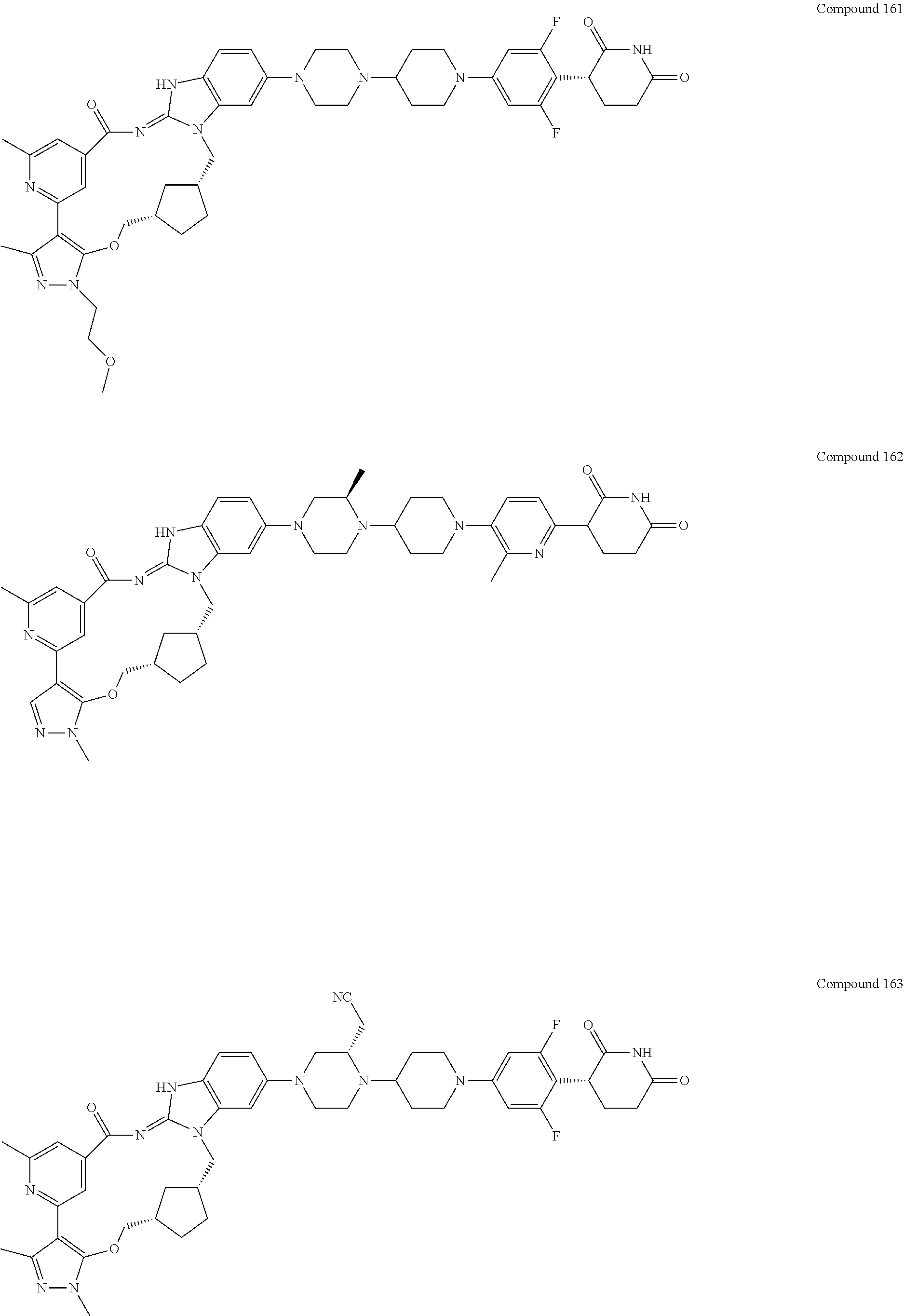 [SMILES: COCCn1nc(C)c2c1OC[C@H]1CC[C@H](C1)CN1/C(=N/C(=O)c3cc(C)nc-2c3)Nc2ccc(N3CCN(C4CCN(c5cc(F)c([C@H]6CCC(=O)NC6=O)c(F)c5)CC4)CC3)cc21 ; Cc1cc2cc(n1)-c1cnn(C)c1OC[C@H]1CC[C@H](C1)CN1/C(=N/C2=O)Nc2ccc(N3CCN(C4CCN(c5ccc(C6CCC(=O)NC6=O)nc5C)CC4)[C@H](C)C3)cc21 ; Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)[C@@H](CC#N)C3)cc21]

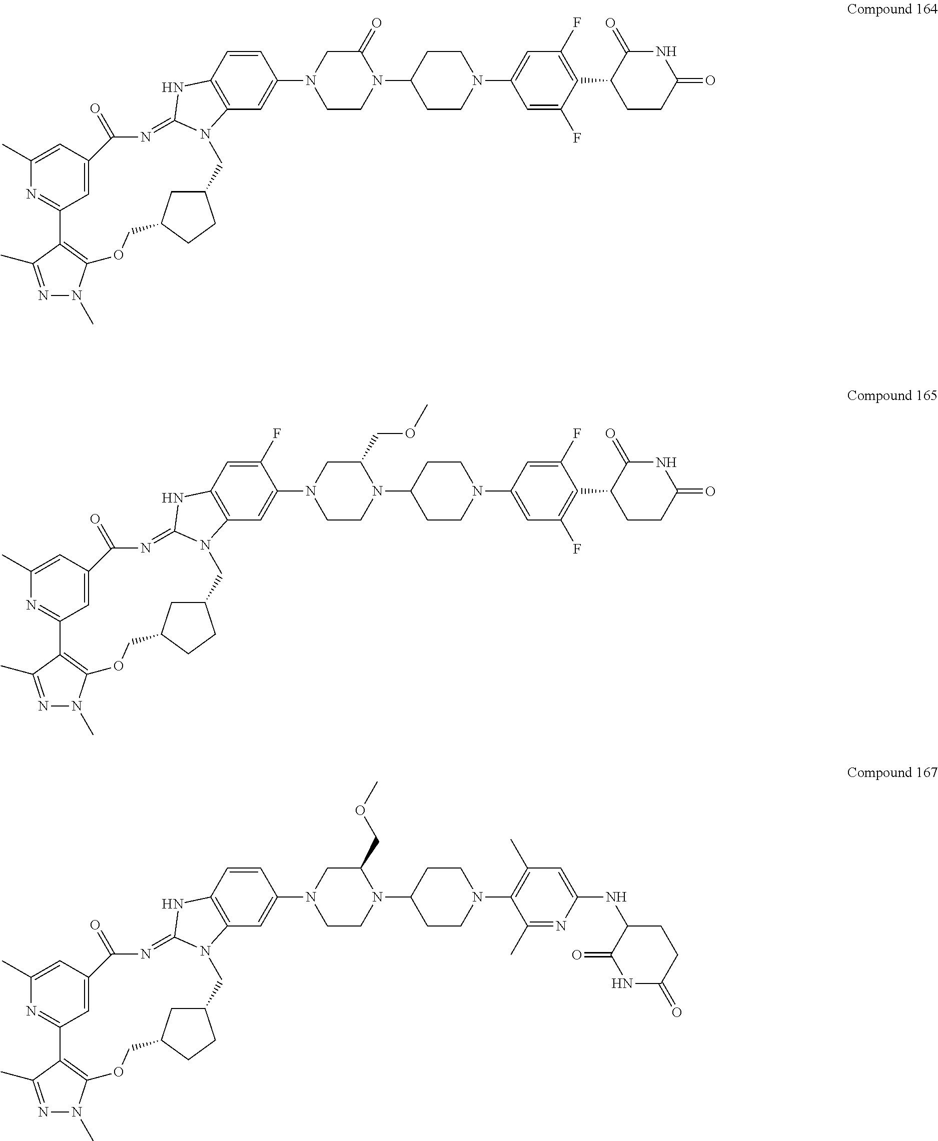 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C(=O)C3)cc21 ; COC[C@H]1CN(c2cc3c(cc2F)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1 ; COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(NC3CCC(=O)NC3=O)nc2C)CC1]

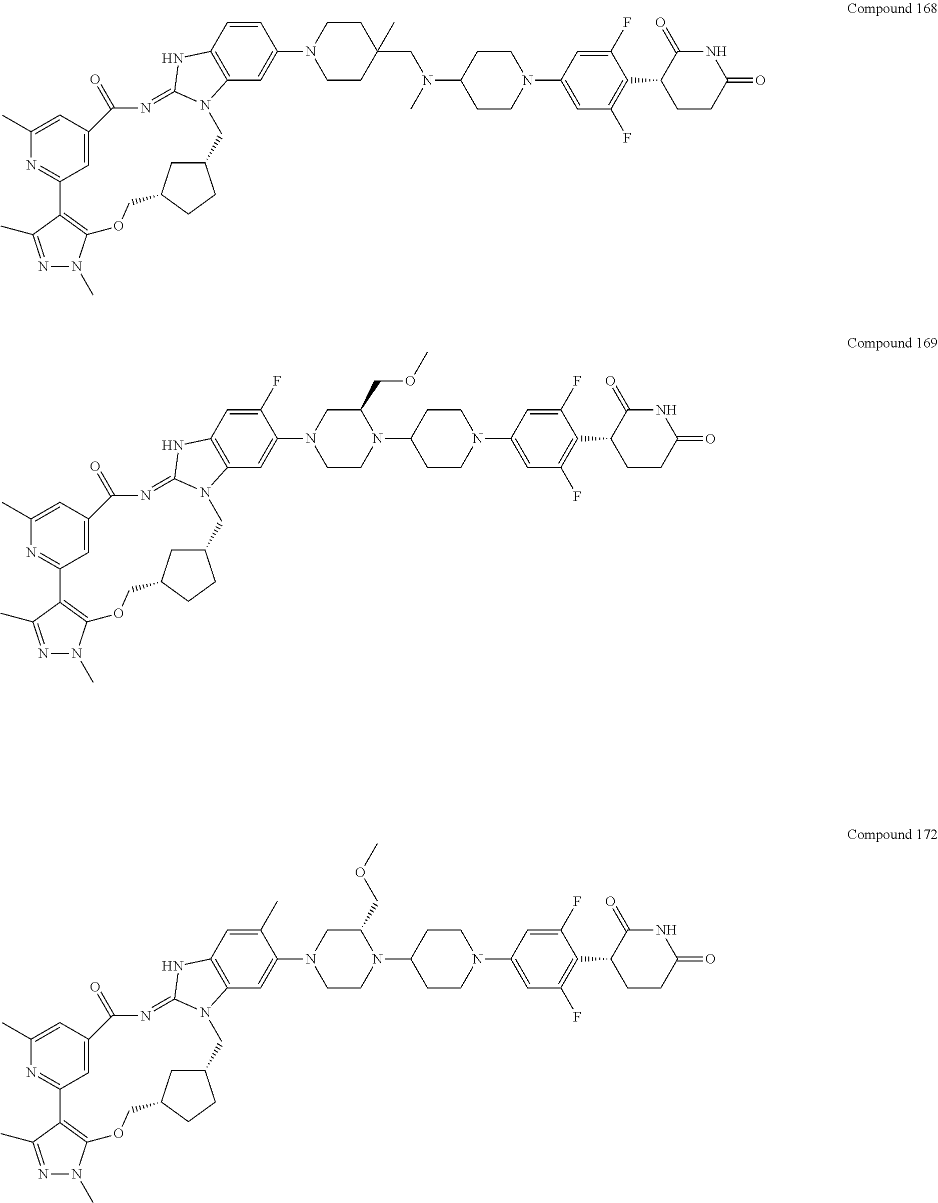 [SMILES: Cc1cc2cc(n1)-c1c(C)nn(C)c1OC[C@H]1CC[C@H](C1)CN1/C(=N/C2=O)Nc2ccc(N3CCC(C)(CN(C)C4CCN(c5cc(F)c([C@H]6CCC(=O)NC6=O)c(F)c5)CC4)CC3)cc21 ; COC[C@@H]1CN(c2cc3c(cc2F)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1 ; COC[C@H]1CN(c2cc3c(cc2C)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

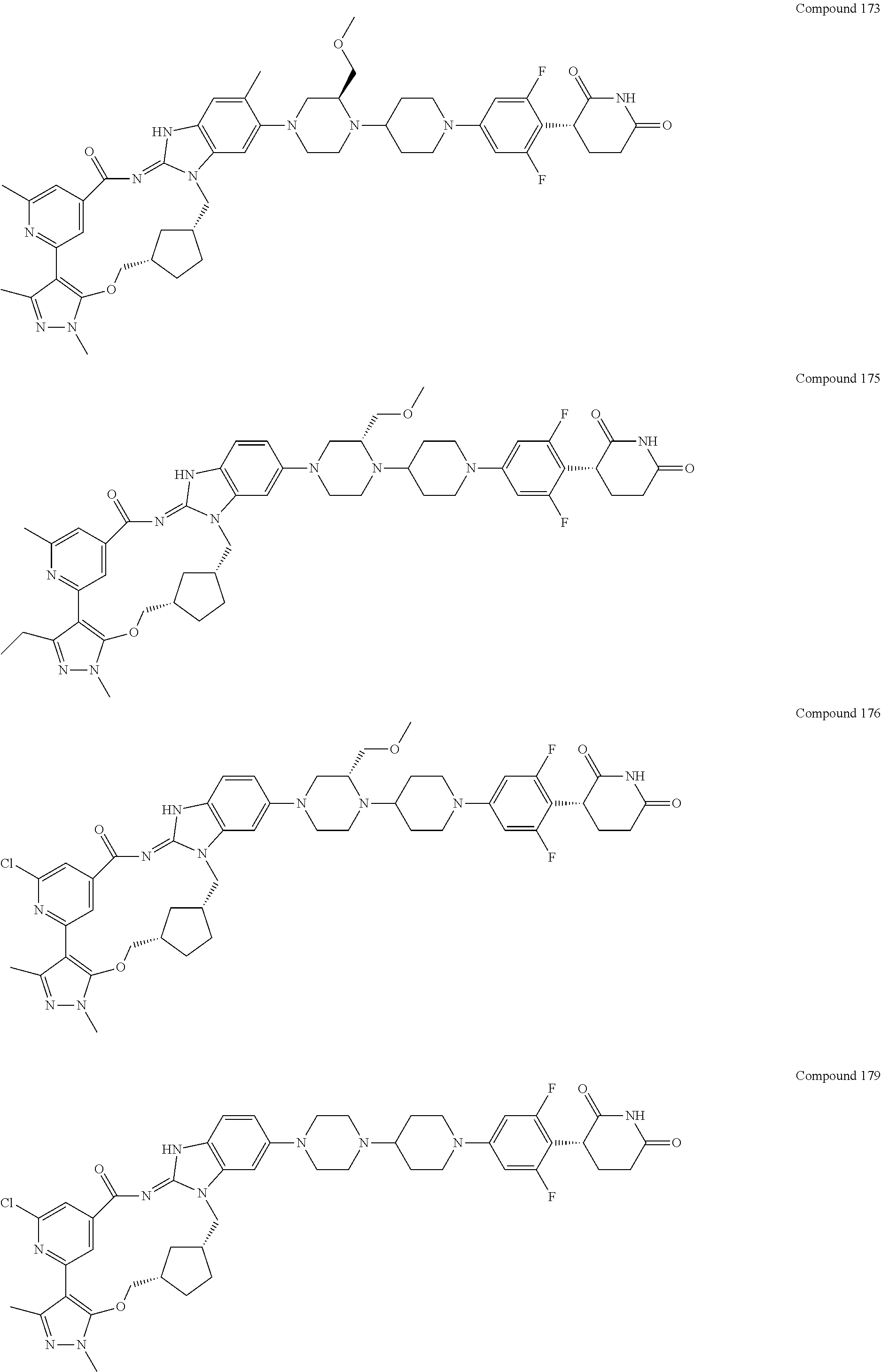

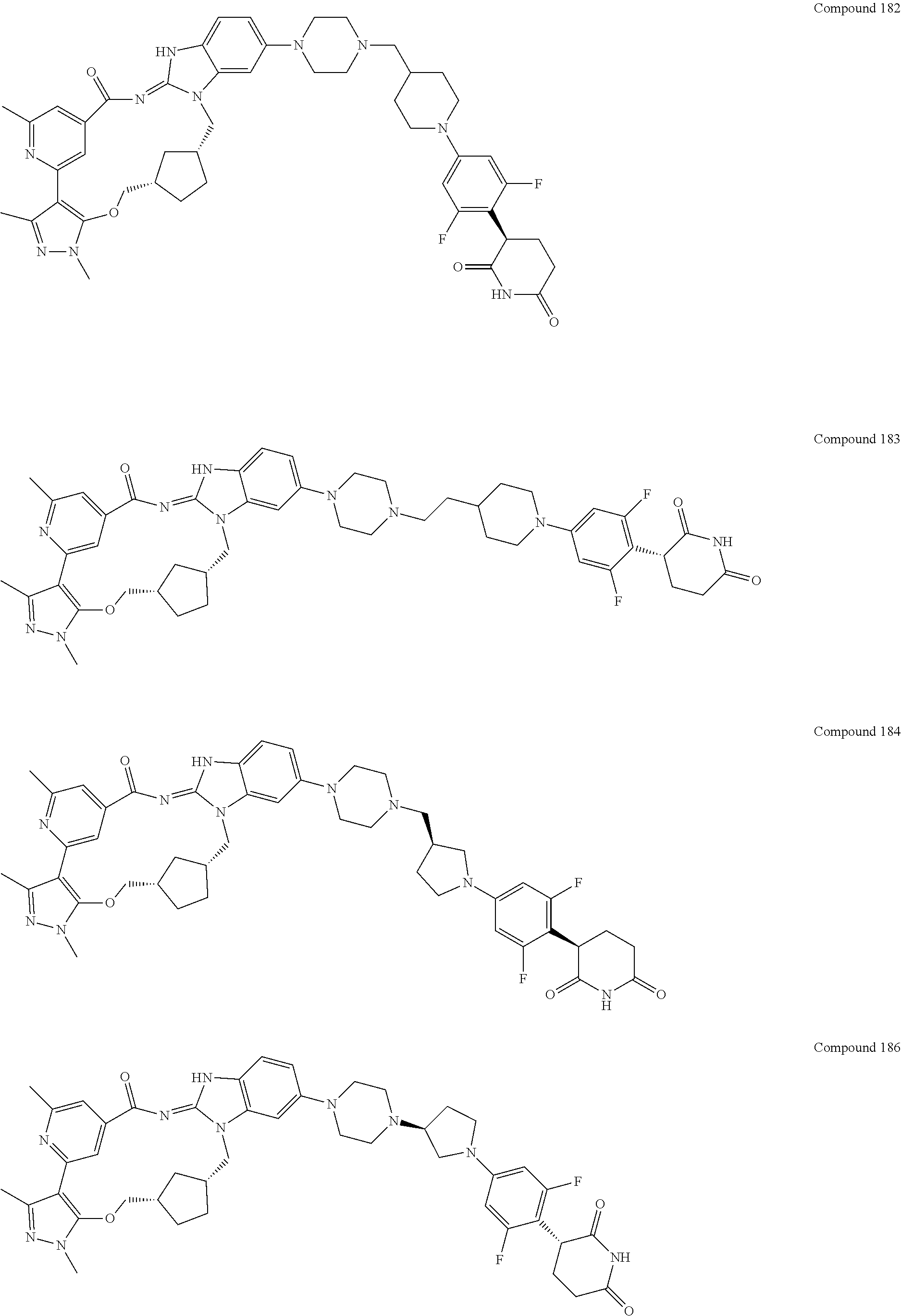

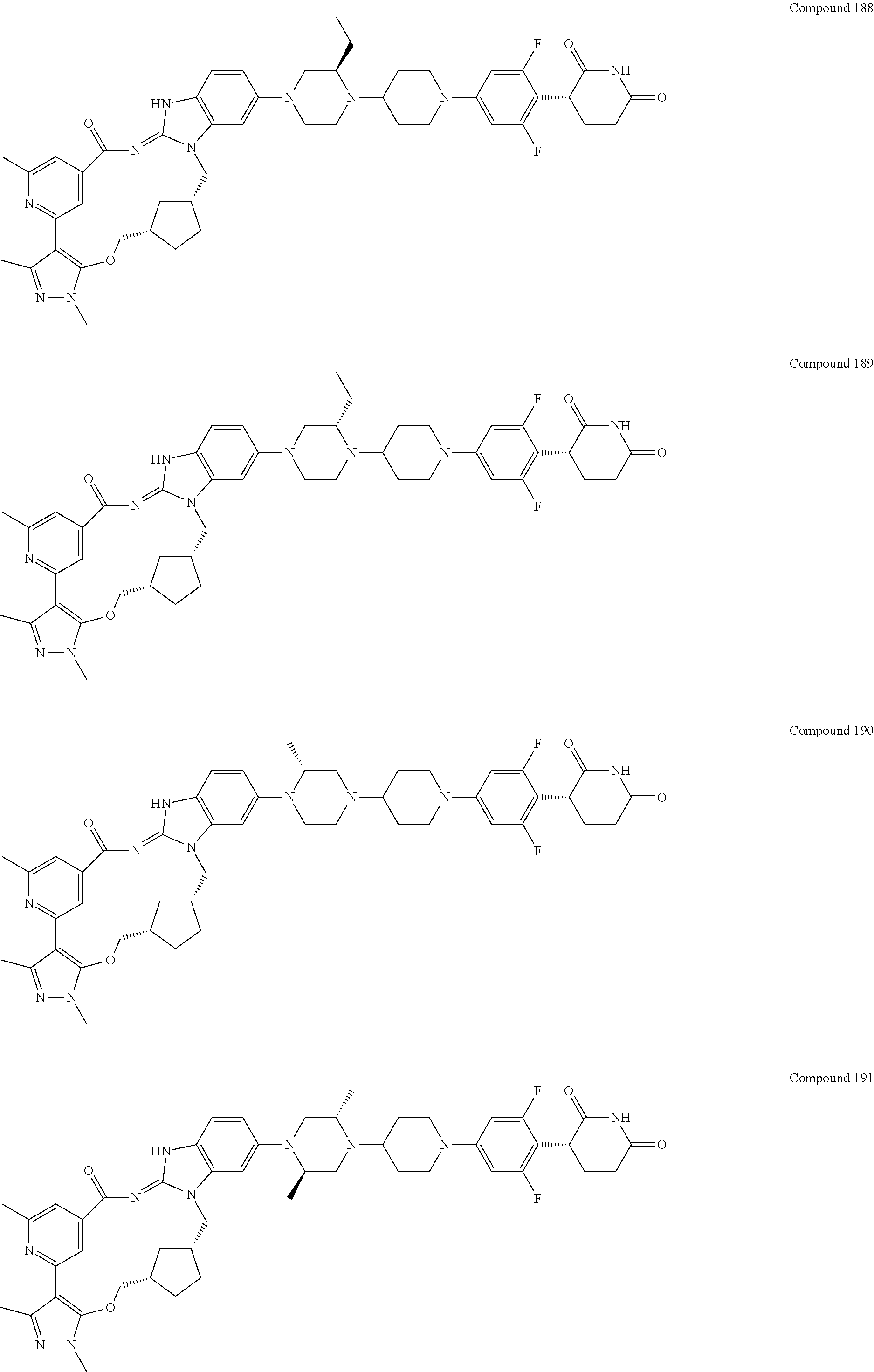

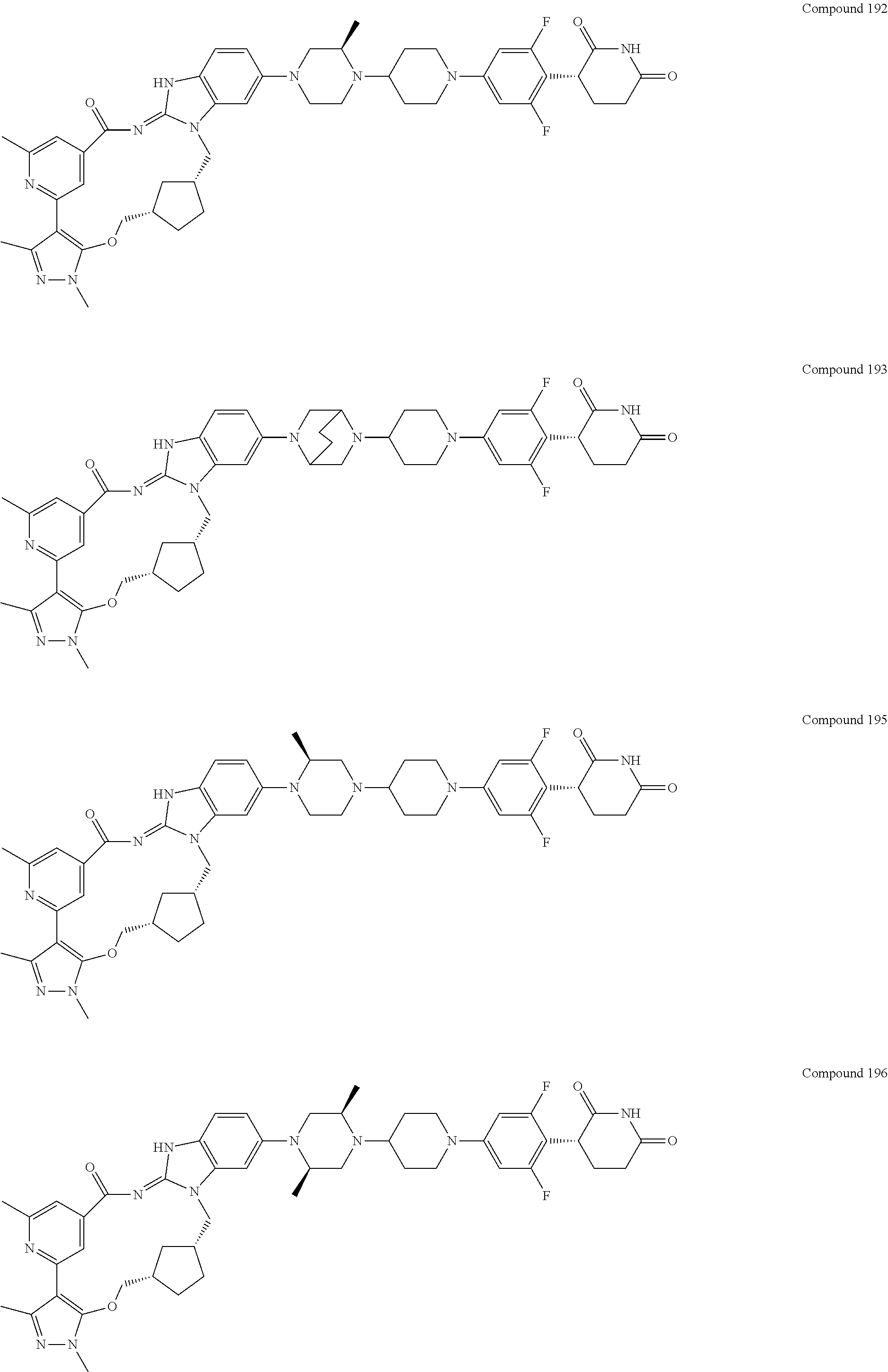

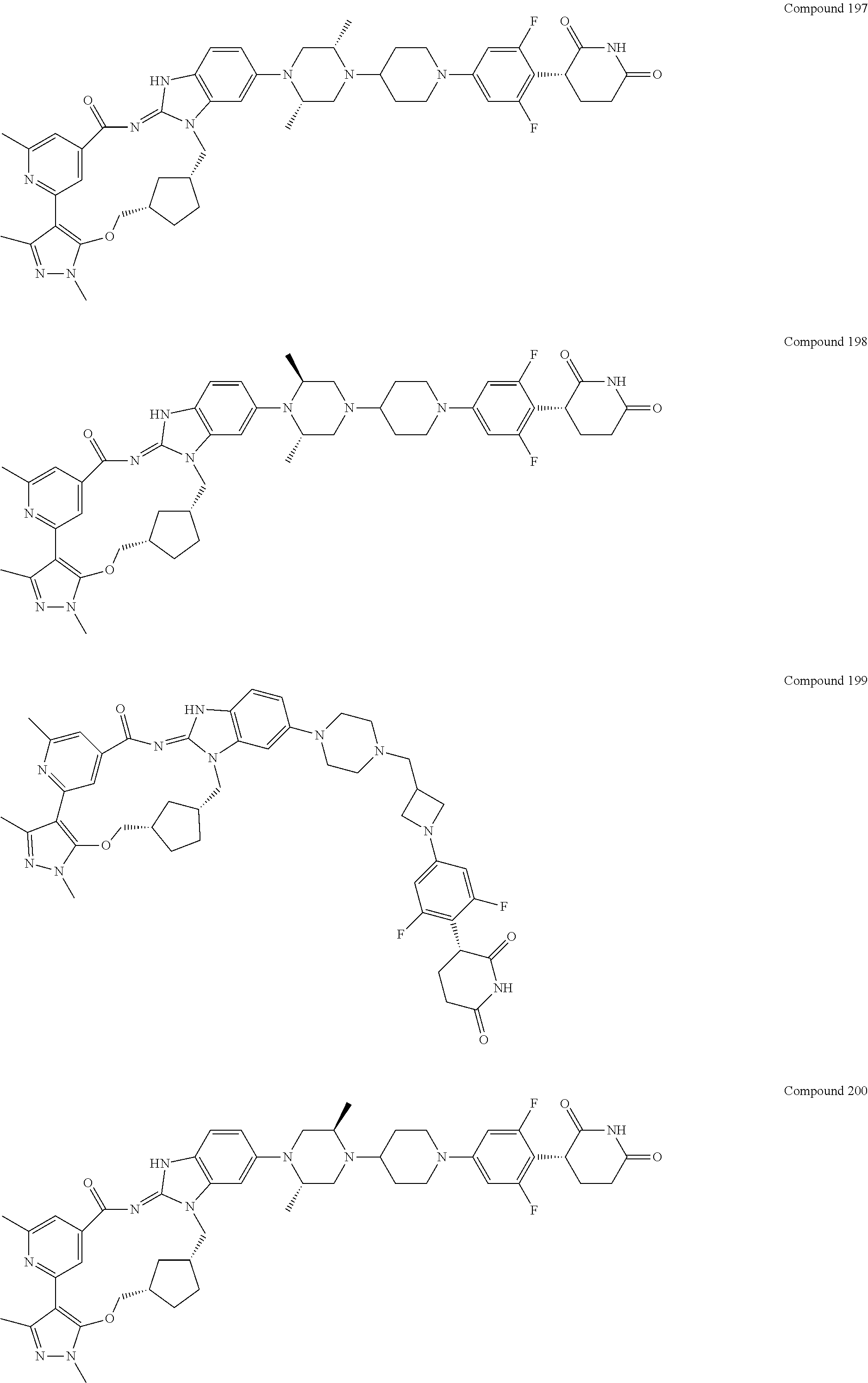

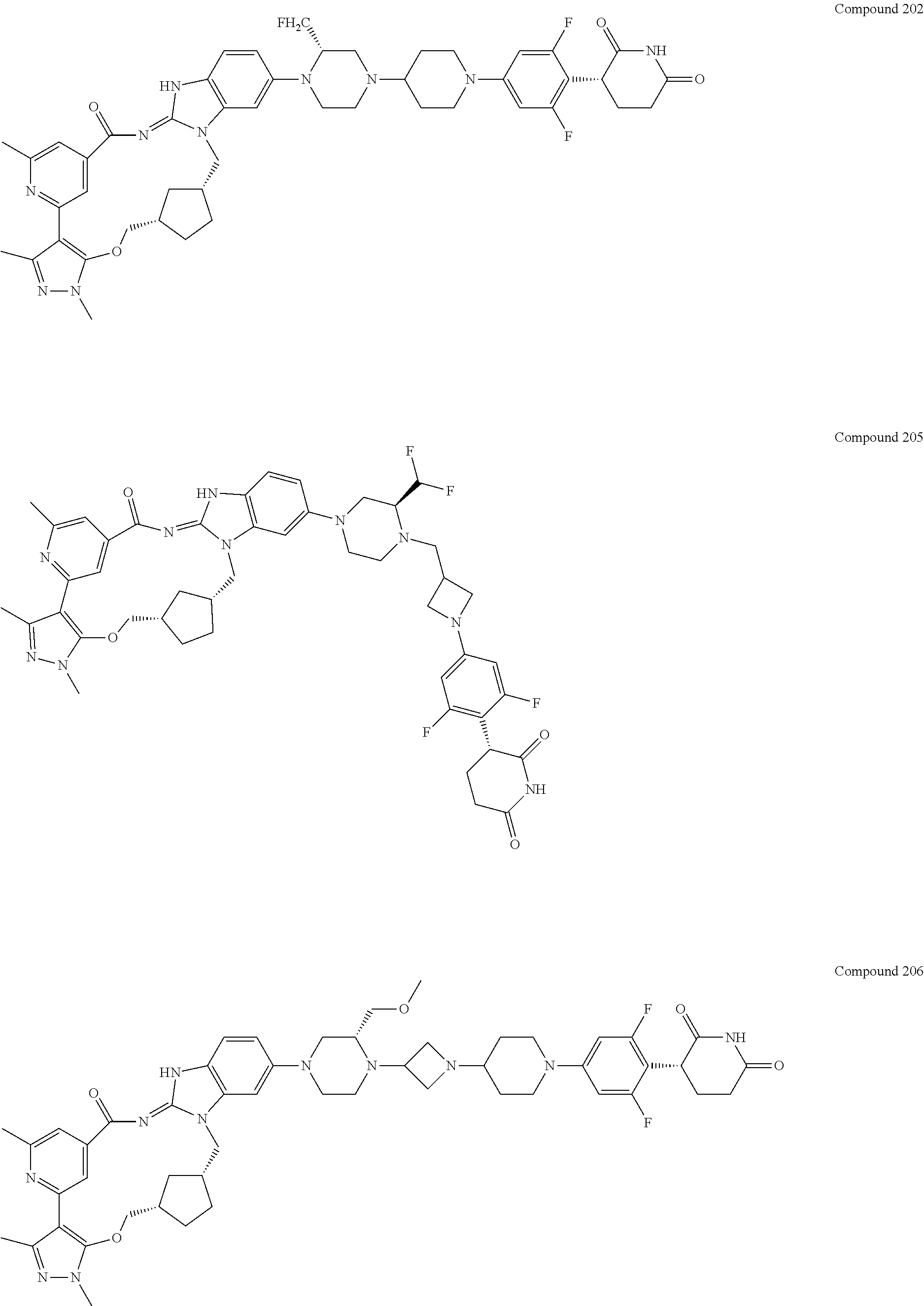

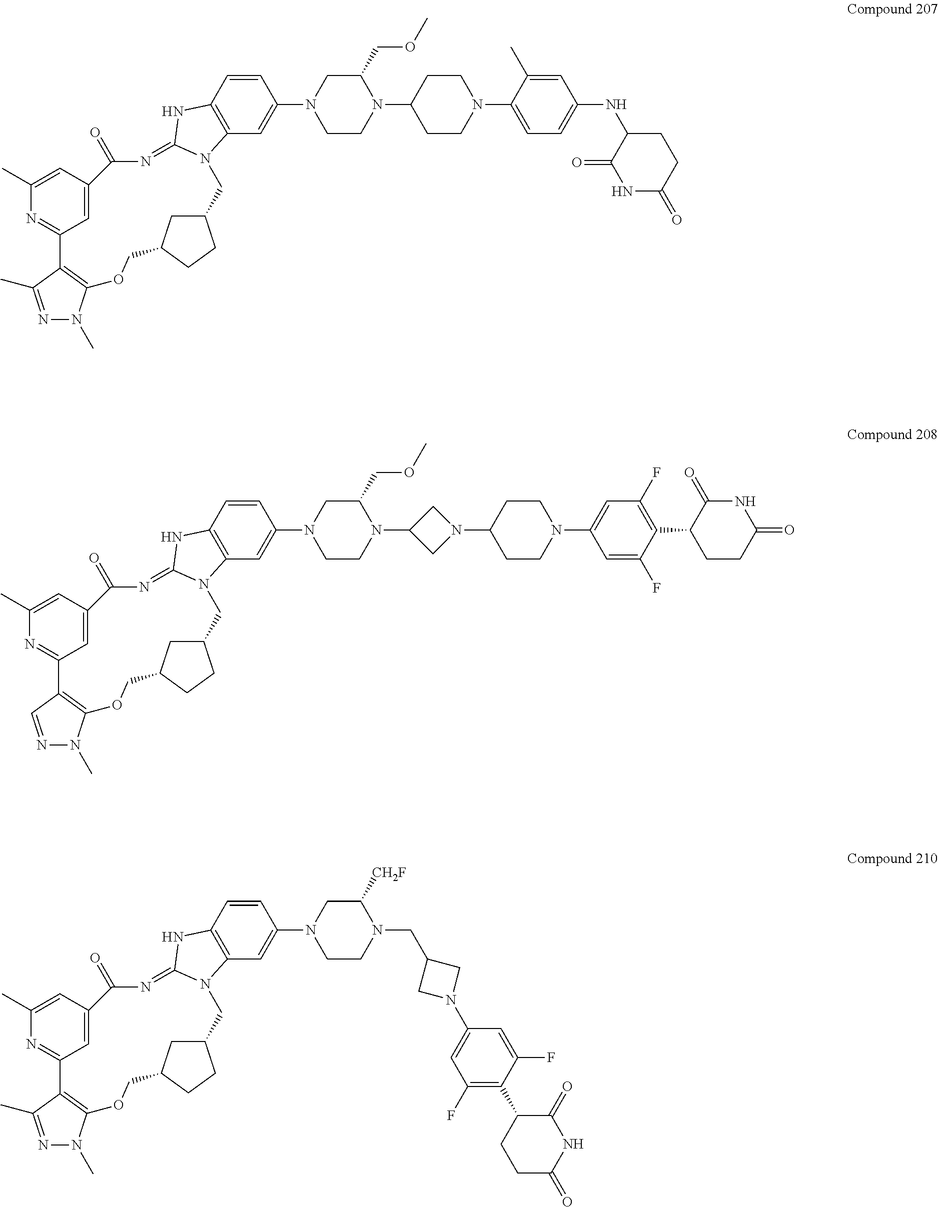

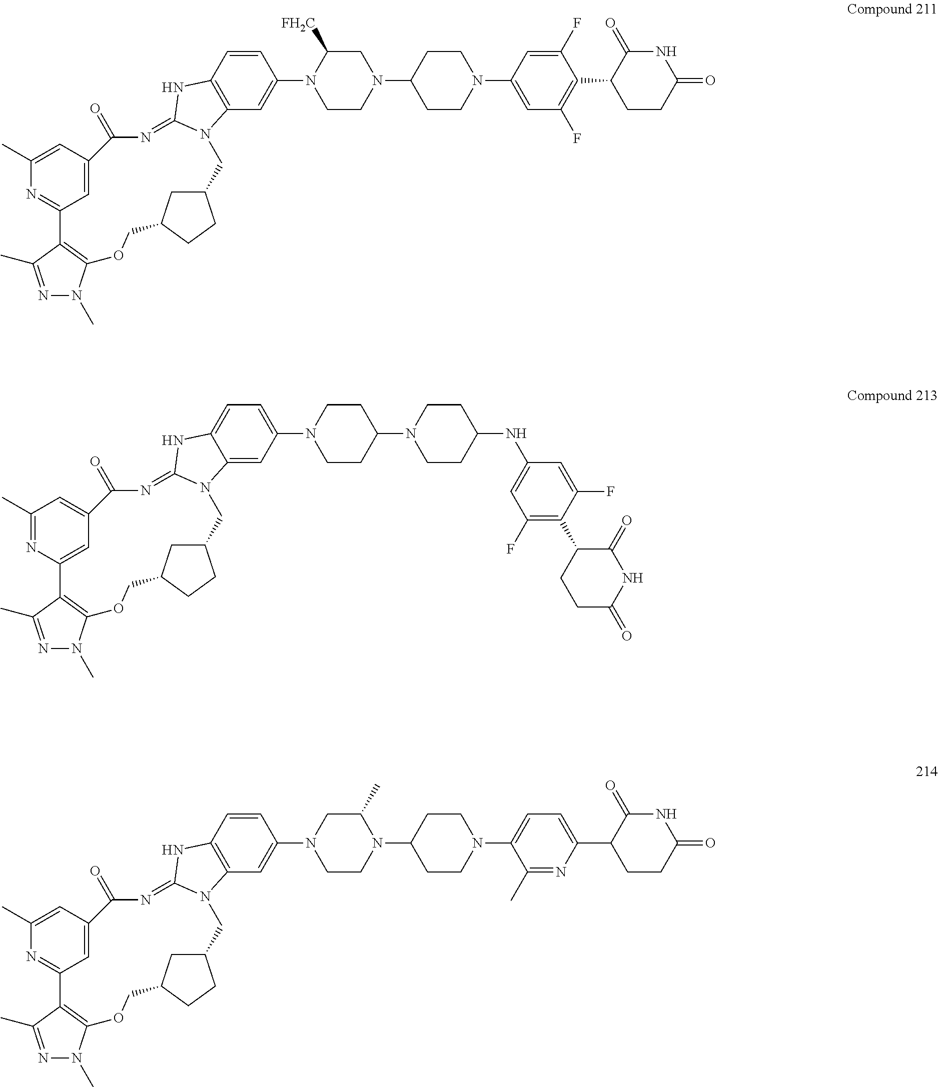

Aspect 36. A pharmaceutical composition comprising a compound of any one of Aspects 1-35 or an N-oxide, pharmaceutically acceptable salt, stereoisomer, tautomer, deuterated analog, or a prodrug, together with pharmaceutically acceptable excipients.

In one embodiment, A pharmaceutical composition comprising a compound of any one of Aspects 1-35 or a pharmaceutically acceptable salt, stereoisomer, or tautomer, together with pharmaceutically acceptable excipients.
In one embodiment, A pharmaceutical composition comprising a compound of any one of Aspects 1-35 together with pharmaceutically acceptable excipients. In one embodiment, A pharmaceutical composition comprising a compound of any one of Aspects 1-35 or a pharmaceutically acceptable salt together with pharmaceutically acceptable excipients. In one embodiment, A pharmaceutical composition comprising a compound of any one of Aspects 1-35 or a pharmaceutically acceptable stereoisomer together with pharmaceutically acceptable excipients. In one embodiment, A pharmaceutical composition comprising a compound of any one of Aspects 1-35 or a pharmaceutically acceptable tautomer together with pharmaceutically acceptable excipients.

Aspect 37. A method of treating a disease that can be affected by EGFR modulation, comprises administrating a subject in need thereof an effective amount of a compound of any one of Aspects 1-35 or an N-oxide, pharmaceutically acceptable salt, stereoisomer, tautomer, deuterated analog, or a prodrug thereof.

In one embodiment, A method of treating a disease that can be affected by EGFR modulation, comprises administrating a subject in need thereof an effective amount of a compound of any one of Aspects 1-35 or a pharmaceutically acceptable salt, stereoisomer, or a tautomer thereof.
In one embodiment, A method of treating a disease that can be affected by EGFR modulation, comprises administrating a subject in need thereof an effective amount of a compound of any one of Aspects 1-35. In one embodiment, A method of treating a disease that can be affected by EGFR modulation, comprises administrating a subject in need thereof an effective amount of a compound of any one of Aspects 1-35 or a pharmaceutically acceptable salt thereof. In one embodiment, A method of treating a disease that can be affected by EGFR modulation, comprises administrating a subject in need thereof an effective amount of a compound of any one of Aspects 1-35 or a pharmaceutically acceptable stereoisomer thereof. In one embodiment, A method of treating a disease that can be affected by EGFR modulation, comprises administrating a subject in need thereof an effective amount of a compound of any one of Aspects 1-35 or a pharmaceutically acceptable tautomer thereof.

Aspect 38. The method of Aspect 37, wherein the disease is selected from cancer, preferred pancreatic cancer, breast cancer, glioblastoma multiforme, head and neck cancer, or non-small cell lung cancer.

Aspect 39. Use of a compound of any one of Aspects 1-35 or a pharmaceutically acceptable salt, stereoisomer, tautomer or prodrug thereof in the preparation of a medicament for treating a disease that can be affected by EGFR modulation.

Aspect 40. The use of Aspect 39, wherein the disease is cancer, preferred pancreatic cancer, breast cancer, glioblastoma multiforme, head and neck cancer, or non-small cell lung cancer.

The 5-membered ring moiety

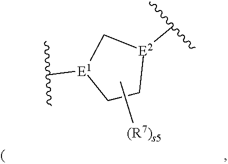

preferably

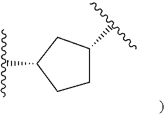 [SMILES: *[C@@H]1CC[C@H](*)C1]

makes the molecules of this application have much lower toxicity than the 3- or 4-membered ring molecules. The toxicity can be tested by the common methods of the field. The 5-membered ring moiety

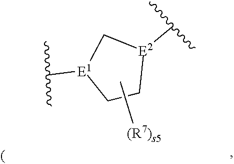

preferably

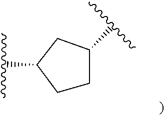 [SMILES: *[C@@H]1CC[C@H](*)C1]

makes the molecules of this application have much better activity on L858R mutation and L858R/C797S double mutation of EGFR than the 3- or 4-membered ring molecules. At the same time, the 5-membered ring moiety

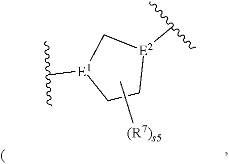

preferably

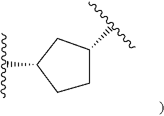 [SMILES: *[C@@H]1CC[C@H](*)C1]

makes the molecules of this application have comparable or better activity on Del19 single mutation, Del19/C797S double mutation, Del19/T790M/C797S triple mutation and L858R/T790M/C797S triple mutation of EGFR with the 3- or 4-membered ring molecules. The degradation activity can be tested by the methods recorded in this application. The 5-membered ring moiety

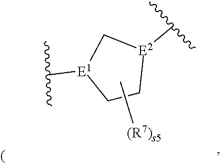

preferably

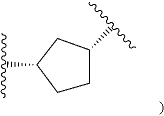 [SMILES: *[C@@H]1CC[C@H](*)C1]

makes the molecules of this application have better rat PK than the 3- or 4-membered ring molecules.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms. Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

The term “aryl” used alone or in combination with other terms includes a group selected from: 5- and 6-membered carbocyclic aromatic rings, e.g., phenyl; bicyclic ring systems such as 7 to 12 membered bicyclic ring systems, wherein at least one ring is carbocyclic and aromatic, e.g., naphthyl and indanyl; and, tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable. For another example, compounds including pyrazolyl may undergo tautomerism to form a different ring like below:

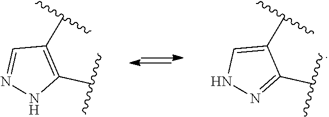 [SMILES: *c1cn[nH]c1* ; *c1c[nH]nc1*]

For another example, compounds including guanidinyl in the ring may undergo tautomerism to form a different ring like below:

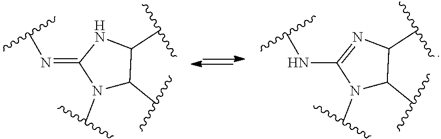 [SMILES: */N=C1\NC(*)C(*)N1* ; *NC1=NC(*)C(*)N1*]

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Deuterated analog” refers to a derivative of an active agent that an arbitrary ¹H atom is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “treated”, “treating” or “treatment” as used herein also generally refers to the acquisition of the desired pharmacological and/or physiological effect. The effect may be prophylactic according to the prevention of the disease or its symptoms in whole or in part; and/or may be therapeutic according to the partial or complete stabilization or cure of the disease and/or the side effect due to the disease. As used herein, “treated”, “treating” or “treatment” encompasses any treatment for the disease of a patient, including: (a) prevention of the disease or condition in the patient that may be predisposed to the disease or condition but has not yet been diagnosed; (b) inhibition of the symptoms of the disease, i.e., preventing its development; or (c) remission of the symptoms of the disease, i.e., causing regression of the disease or symptoms in whole or in part.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless otherwise specified, the percentages, proportions, ratios or parts used in the present application are by weight or volume. It can be determined easily by those skilled in the art.

Hereinafter, the present application will demonstrate the beneficial effects of the present application by way of examples. Those skilled in the art will recognize that these examples are illustrative and not restrictive. These examples do not limit the scope of the present application in any way. The experimental methods described in the following examples, unless otherwise specified, are conventional methods; the reagents and materials, unless otherwise specified, are commercially available.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

1H NMR spectra were recorded on an Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-3: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (0)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below may be useful:

| | |
|---|---|
| (BPin)2 | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| Cbz | Benzyloxycarbonyl |
| DCM | Dichloromethane |
| DBU | 1,8-diazabicyclo [5.4.0]undec-7-ene |
| DCE | dichloroethane |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h, hr or hrs | Hour |
| HATU | 2-(7-Azabenzotriazol-1-yl)-N,N,N′,N′-tetramethyluronium |
| | hexafluorophosphate |
| Hex | Hexane |
| HPLC | High Performance Liquid Chromatography |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCI | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| Johnphos | (2-Biphenyl)di-tert-butylphosphine |
| TCFH | N,N,N′,N′-Tetramethylchloroformamidinium |
| | hexafluorophosphate |
| CDI | 1,1′-Carbonyldiimidazole |
| aq. | Aqueous |
| DCE | Dichloroethane |
| DEA | N,N-Diethylaniline |
| DIAD | Diisopropyl azodicarboxylate |
| DMA | N,N-Dimethylacetamide |
| DMC | Dimethyl carbonate |
| LCMS | Liquid Chromatography Mass Spectrometry |
| LDA | Lithium diisopropylamide |
| NCS | N-Chlorosuccinimide |
| NMR | Nuclear Magnetic Resonance |
| dtbpf | 1,1′-Bis(di-tert-butylphosphino)ferrocene |
| Pd(dtbpf)Cl₂ | [1,1′-Bis(di-tert-butylphosphino)ferrocene]dichloropalladium(II) |
| Pd-PEPPSI-IPentCl | [1,3-bis[2,6-bis(1-propylbutyl)phenyl]-4,5-dichloro-imidazol-2- |
| | ylidene]-dichloro-(3-chloropyridin-1-ium-1-y1)palladium |
| PPh₃ | triphenylphosphine |
| PPh₃O | Triphenylphosphine oxide |
| Prep-HPLC | Preparative-scale High-Performance Liquid Chromatography |
| RuPhos | 2-Dicyclohexylphosphino-2′,6′-diisopropoxy-1,1′-biphenyl |
| RuPhos Pd G3 | Methanesulfonato(2-dicyclohexylphosphino-2′,6′-di-isopropoxy- |
| | 1,1′-biphenyl)(2′-amino-1,1′-biphenyl-2-yl)palladium(II) |
| T₃P | Propylphosphonic anhydride |
| 9-BBN | 9-borabicyclo[3.3.1]nonane |
| CAN | Ammonium cerium(IV) nitrate |
| DEA | Diethylamine |
| Del19 | exon 19 deletion |
| DIAD | Diisopropyl azodicarboxylate |
| DMA | N,N-Dimethylacetamide |
| FBS | Fetal bovine serum |
| HTRF | Homogeneous Time Resolved Fluorescence |
| LDA | Lithium diisopropylamide |
| Ms | Mesylate |
| MtBE | tert-Butyl methyl ether |
| NCS | N-Chlorosuccinimide |
| NMP | 1-Methyl-2-Pyrrolidone |
| NMR | Nuclear Magnetic Resonance |
| Pd(PPh3)2Cl2 | Bis(triphenylphosphine)palladium(II) dichloride |
| Pd(PPh3)4 | Tetrakis(triphenylphosphine)palladium(0) |
| Pd2(dba)3 | Tris(dibenzylideneacetone)dipalladium(0) |
| Pd-PEPPSI-IPentCl | [1,3-bis[2,6-bis(1-propylbutyl)phenyl]-4,5-dichloro-imidazol-2- |
| | ylidene]-dichloro-(3-chloropyridin-1-ium-1-yl)palladium |
| Pd-Ruphos-G3 or Pd- | (2-Dicyclohexylphosphino-2′,6′-diisopropoxy-1,1′-biphenyl)[2- |
| G3 RuPhos or RuPhos | (2′-amino-1,1′-biphenyl)]palladium(II) methanesulfonate |
| Pd G3 | |
| PE | Petroleum ether |
| PPh3 | Triphenylphosphine |
| Prep-SFC | preparative supercritical fluid chromatography |
| PS | Penicillin-Streptomycin Solution |
| RT | room temperature |
| Ruphos | {2-[2,6-bis(propan-2- |
| | yloxy)phenyl]phenyl}dicyclohexylphosphane |
| t-BuONO | tert-Butyl nitrite |

### General Synthesis for the Intermediates

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art. In the synthesis schemes, some protection/deprotection steps are not shown and can be incorporated before, after or in between any steps. The protecting group shown in the synthesis schemes may or may not be used based on reaction conditions. The sequences of reactions may vary and provide similar results.

Reactions can be monitored according to any suitable method known in the art, such as NMR, UV, HPLC, LC-MS and TLC. Compounds can be purified by a variety of methods, including prep-HPLC and silica gel chromatography. Unless specified, prep-HPLC uses a buffered acetonitrile/water systems and silica gel chromatography (including column chromatography and prep-TLC) uses PE/EtOAc, EtOAc/MeOH or DCM/MeOH systems as mobile phases. NMR spectra are recorded using a Bruker or Varian instrument with preset pulse sequences.

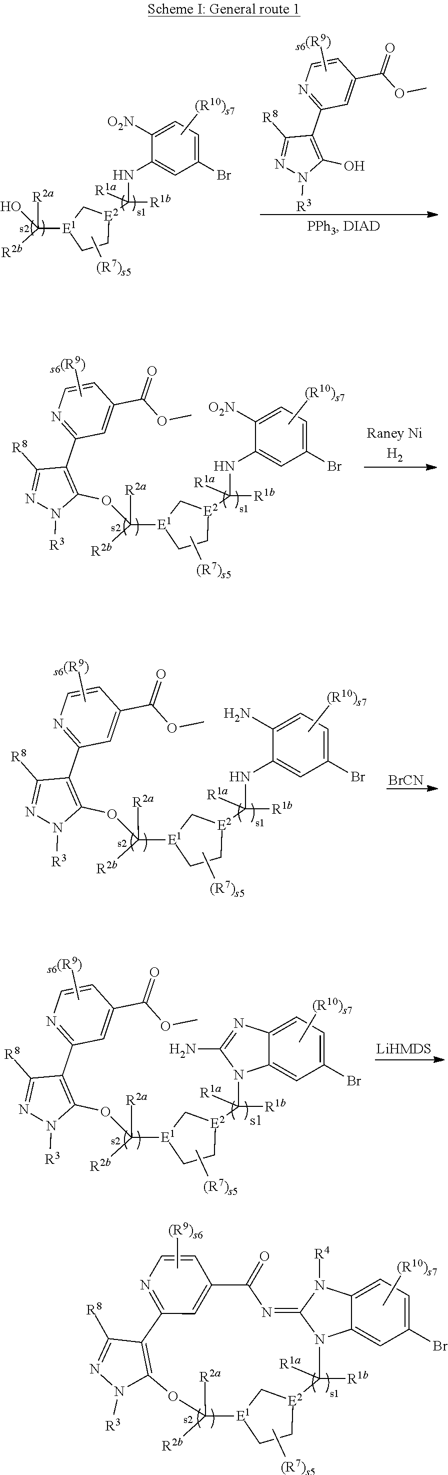

Intermediates can be synthesized through General route 1, such as Intermediate 1, 57 or 75.

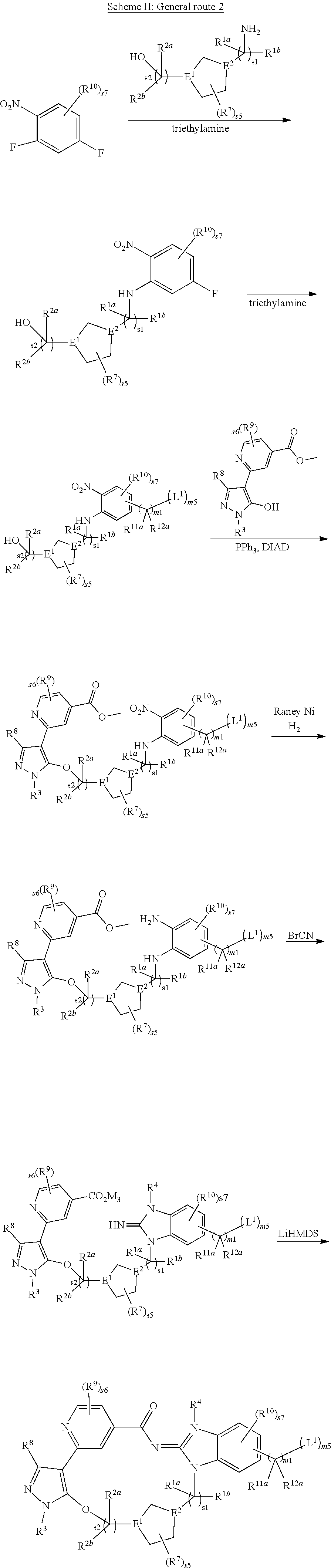

Intermediates can be synthesized through General route 2, such as Intermediate 135.

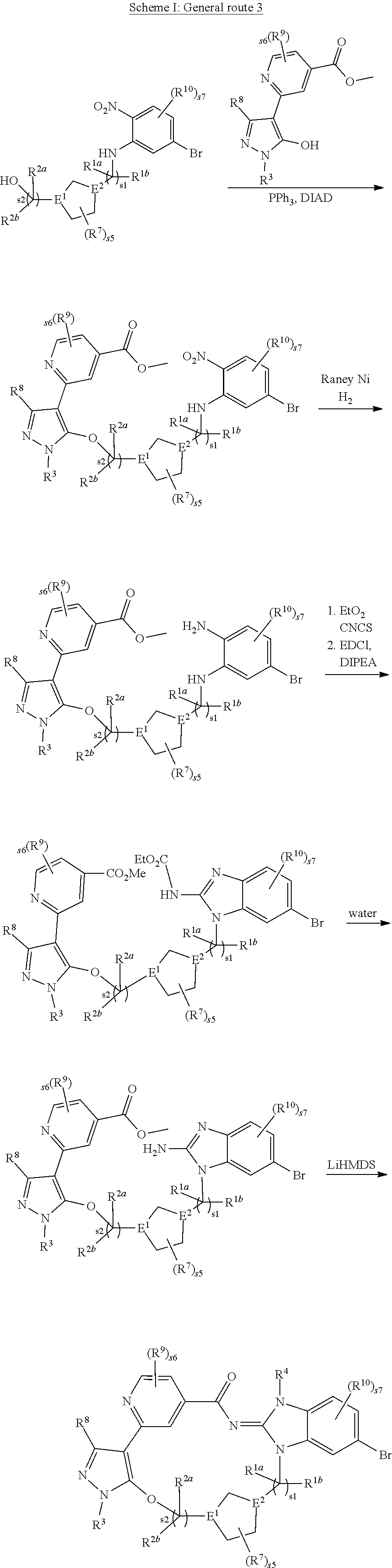

Intermediates can be synthesized through General route 3, such as Intermediate 134.

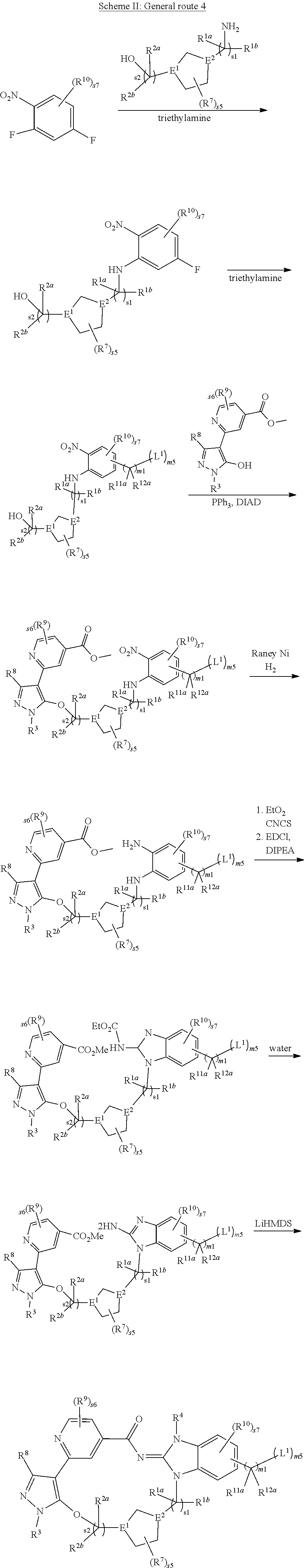

Intermediates can be synthesized through General route IV, such as Intermediate 137.

### Intermediate Synthesis

### Intermediate 1: (7¹R,7³S,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

### Step 1: 3-oxabicyclo[3.2.1]octane-2,4-dione

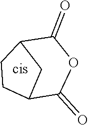

A 2 L round bottom flask equipped with a magnetic stirrer were charged with cis-cyclopentane-1,3-dicarboxylic acid (100 g, 633 mmol) and Ac₂O (600 ml). The mixture was stirred for 12 hours at 140° C. The mixture was concentrated under reduced pressure. The residue was triturated with PE to afford the crude product (70 g, 496.4 mmol, 78.5%). ¹H NMR (400 MHz, DMSO) δ 3.18 (q, J=3.9 Hz, 2H), 2.35 (d, J=12.4 Hz, 1H), 2.17-2.01 (m, 2H), 1.90 (t, J=7.5 Hz, 2H), 1.69 (dt, J=12.4, 4.2 Hz, 1H).

### Step 2: cis-3-carbamoylcyclopentane-1-carboxylic acid

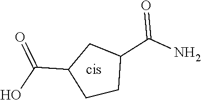 [SMILES: NC(=O)C1CCC(C(=O)O)C1]

A 2 L round bottom flask equipped with a magnetic stirrer were charged with (1R,5S)-3-oxabicyclo[3.2.1]octane-2,4-dione (70 g, 496.4 mmol), THF (700 ml) and NH₃-MeOH 7M (4 eq) at 0° C. The mixture was stirred for 12 hours at RT. The residue was quenched by MeOH (500 ml) at 0° C. The mixture was concentrated under reduced pressure to afford crude product (56 g, 354.4 mmol, 74.4%). [M+H]⁺=158.

### Step 3: methyl cis-3-carbamoylcyclopentane-1-carboxylate

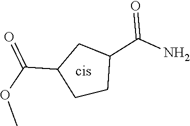

A 1 L round bottom flask equipped with a magnetic stirrer, were charged with cis-3-carbamoylcyclopentane-1-carboxylic acid (56 g, 354.4 mmol) and MeOH (600 mL). The temperature was lowered to 0° C., and H₂SO₄ (34.7 g, 354 mmol) was added dropwise into the mixture while maintaining temperature at 0° C. The resulting mixture was stirred at RT for 12 hrs. The mixture was concentrated under reduced pressure to afford crude product (49 g, 284.8 mmol, 80.3%). [M+H]⁺=172.

### Step 4: ((cis)-3-(aminomethyl)cyclopentyl)methanol

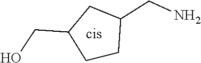

A 1 L round bottom flask equipped with a magnetic stirrer, were charged with methyl cis-3-carbamoylcyclopentane-1-carboxylate (49 g, 284.8 mmol) and THF (500 mL). The temperature was lowered to 0° C., and BH₃-THF (1140 ml, 1140 mmol) was added dropwise into the mixture while maintaining temperature at 0° C. The resulting mixture was stirred at RT for 24 hrs. The residue was quenched by MeOH (500 ml) at 0° C. The mixture was concentrated under reduced pressure to afford crude product (35 g, 269.2 mmol, 79.5%). [M+H]⁺=130.

### Step 5: ((cis)-3-(((5-bromo-2-nitrophenyl)amino)methyl)cyclopentyl)methanol

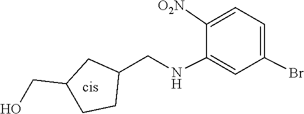 [SMILES: O=[N+]([O-])c1ccc(Br)cc1NCC1CCC(CO)C1]

A 1 L round bottom flask equipped with a magnetic stirrer, were charged with ((cis)-3-(aminomethyl)cyclopentyl)methanol (35 g, 269.2 mmol), 4-bromo-2-fluoro-1-nitrobenzene (64.8 g, 295.9 mmol), DIEA (104.2 g, 807.8 mmol), and DMSO (500 mL). The mixture was stirred at 80° C. under N₂ for 4 hrs. After cooled to room temperature, the mixture was poured into EA (500 mL), and washed with brine (200 mL), water (3′200 mL), brine (200 mL) in turn, then dried over anhydrous Na₂SO₄, concentrated in vacuum. The residue was purified by column chromatography (EA/PE, 24%) to afford the title product (20 g, 60.8 mmol, 22.6%). [M+H]⁺=329.

### Step 6: ((1S,3R)-3-(((5-bromo-2-nitrophenyl)amino)methyl)cyclopentyl)methanol

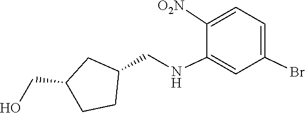 [SMILES: O=[N+]([O-])c1ccc(Br)cc1NC[C@@H]1CC[C@H](CO)C1]

The crude product (20 g) was purified by Prep-SFC with the following conditions ((Lux 3u Cellulose-3 4.6*50 mm, 3 m), Temperature: 35° C., Flow (mL/min): 4, solvent A: CO₂, solvent B: IPA (0.5% 2 mM NH₃-MeOH), gradient (B %): 10% to 50% in 2.0 min, hold 1.0 min at 50%, retention time 0.971 min) to afford ((1S,3R)-3-(((5-bromo-2-nitrophenyl)amino)methyl)cyclopentyl)methanol (5.6 g, 28.1%, ee=96.08%). [M+H]⁺=329. The crude product could also be purified by chiral HPLC with the following conditions: column (chiralpak IC-3 4.6×50 mm, 3 mm), mobile phase (Hex (0.1% DEA): EtOH=80:20), flow 1.0 mL/min, temp 25° C., retention time 2.836 min.

¹H NMR (300 MHz, DMSO-d6) δ 8.14 (t, J=5.6 Hz, 1H), 7.98 (d, J=9.1 Hz, 1H), 7.27 (d, J=2.1 Hz, 1H), 6.83 (dd, J=9.1, 2.0 Hz, 1H), 4.45 (t, J=5.3 Hz, 1H), 4.34 (s, 1H), 3.43 (s, 1H), 2.23 (dt, J=15.3, 7.5 Hz, 1H), 2.11-1.83 (m, 2H), 1.81-1.54 (m, 1H), 1.45-1.20 (m, 2H), 1.06 (t, J=7.0 Hz, 2H), 1.00-0.71 (m, 1H).

### Step 7: methyl 2-(5-(((1S,3R)-3-(((5-bromo-2-nitrophenyl)amino)methyl)cyclopentyl)methoxy)-1-methyl-1H-pyrazol-4-yl)-6-methylisonicotinate

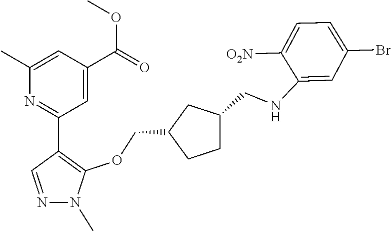

To a stirred solution of ((1S,3R)-3-(((5-bromo-2-nitrophenyl)amino)methyl)cyclopentyl)methanol (5.1 g, 15.5 mmol), methyl 2-(5-hydroxy-1-methyl-1H-pyrazol-4-yl)-6-methylisonicotinate (4.2 g, 17.0 mmol), and PPh₃ (4.9 g, 18.6 mmol) in THF (100 mL) was added DIAD (3.8 g, 18.6 mmol). Then the mixture was stirred for 2 h at room temperature. Then the mixture was concentrated and purified by silica gel column chromatography, eluted with EtOAc/PE (0-80%) to afford the product mixed with PPh₃O (9.3 g); [M+H]+=558.3.

### Step 8: methyl 2-(5-(((1S,3R)-3-(((2-amino-5-bromophenyl)amino)methyl)cyclopentyl)methoxy)-1-methyl-1H-pyrazol-4-yl)-6-methylisonicotinate

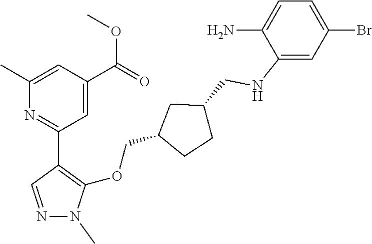 [SMILES: COC(=O)c1cc(C)nc(-c2cnn(C)c2OC[C@H]2CC[C@@H](CNc3cc(Br)ccc3N)C2)c1]

To a stirred solution of methyl 2-(5-(((1S,3R)-3-(((5-bromo-2-nitrophenyl)amino)methyl)cyclopentyl)methoxy)-1-methyl-1H-pyrazol-4-yl)-6-methylisonicotinate (9.3 g, 16.7 mmol) in THF (100 mL) was added Raney nickel (4.5 g). The resulting mixture was stirred under hydrogen atmosphere (1 atm) at room temperature for 2 hr. Then the mixture was filtered and the filtrate was concentrated. The resulting mixture (8.4 g) was used for next step without purification. [M+H]⁺=528.3.

### Step 9: methyl 2-(5-(((1S,3R)-3-((6-bromo-2-imino-2,3-dihydro-1H-benzo[d]imidazol-1-yl)methyl)cyclopentyl)methoxy)-1-methyl-1H-pyrazol-4-yl)-6-methylisonicotinate

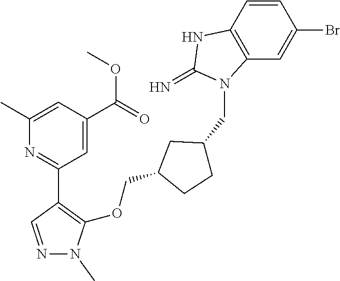 [SMILES: COC(=O)c1cc(C)nc(-c2cnn(C)c2OC[C@H]2CC[C@@H](Cn3c(=N)[nH]c4ccc(Br)cc43)C2)c1]

A solution of methyl 2-(5-(((1S,3R)-3-(((2-amino-5-bromophenyl)amino)methyl)cyclopentyl)methoxy)-1-methyl-1H-pyrazol-4-yl)-6-methylisonicotinate (8.4 g, 15.9 mmol) and BrCN (2.5 g, 23.8 mmol) in MeOH (50 mL) was stirred for 4 h at room temperature. Then the mixture was concentrated and diluted with DCM (200 mL), and then washed with sat. aq. NaHCO₃ (3×100 mL) and brine (100 mL). The organic layer was dried over anhydrous Na₂SO₄, filtered and concentrated under reduced pressure. The residue was purified by silica gel column chromatography, eluted with MeOH/DCM (0-15%) to afford the product (7.1 g, 80.7%); [M+H]⁺=553.4.

### Step 10: (7¹R,7³S,E)-5⁶-bromo-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

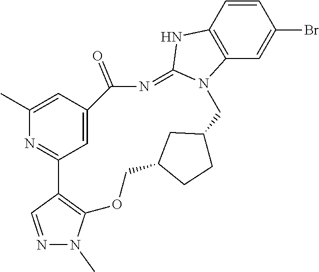 [SMILES: Cc1cc2cc(n1)-c1cnn(C)c1OC[C@H]1CC[C@H](C1)CN1/C(=N/C2=O)Nc2ccc(Br)cc21]

To a stirred solution of methyl 2-(5-(((1S,3R)-3-((6-bromo-2-imino-2,3-dihydro-1H-benzo[d]imidazol-1-yl)methyl)cyclopentyl)methoxy)-1-methyl-1H-pyrazol-4-yl)-6-methylisonicotinate (3.2 g, 5.8 mmol) in 80 mL THF was added LiHMDS (1 N in THF, 14.5 mL) at room temperature for 15 min. The resulting mixture was stirred at room temperature for 1 hr. Then the mixture was diluted with EA (100 mL), washed with sat. aq. NH₄Cl (2×100 mL) and brine (100 mL). The organic layer was dried over anhydrous Na₂SO₄, filtered and concentrated under reduced pressure. The residue was suspended in 100 mL EA and stirred at room temperature for 1 h. Then the mixture was filtered to afford the product (2.6 g, 86.2%); [M+H]⁺=521.2.

### Intermediate 2: (7¹R,7³S,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

### Step 1: tert-butyl 4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate

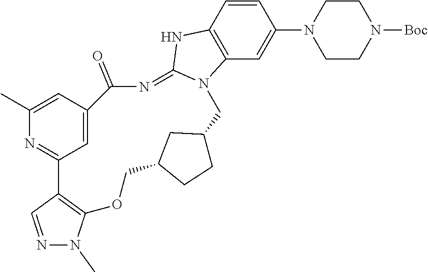 [SMILES: Cc1cc2cc(n1)-c1cnn(C)c1OC[C@H]1CC[C@H](C1)CN1/C(=N/C2=O)Nc2ccc(N3CCN(C(=O)OC(C)(C)C)CC3)cc21]

The solution of intermediate 1 (1.0 g, 1.92 mmol), tert-butyl piperazine-1-carboxylate (535 mg, 2.88 mmol), Pd₂(dba)₃ (360 mg, 0.4 mmol), Ruphos (360 mg, 0.8 mmol) and NaO^{t}Bu (550 mg, 5.75 mmol) in DMA (30 mL) was stirred at 100° C. for 2 hrs. The reaction was concentrated in vacuo, the residue was purified by silica gel column (DCM:CH₃OH=15:1) to afford tert-butyl 4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate (1.0 g, 83.2%). [M+H]⁺=627.

### Step 2: (7¹R,7³S,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

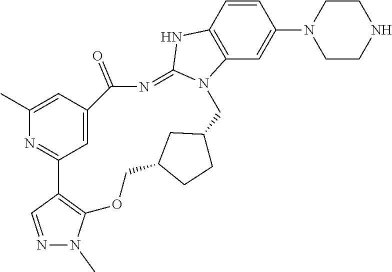 [SMILES: Cc1cc2cc(n1)-c1cnn(C)c1OC[C@H]1CC[C@H](C1)CN1/C(=N/C2=O)Nc2ccc(N3CCNCC3)cc21]

To a mixture of tert-butyl 4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate (1.0 g, 1.6 mmol) in DCM (20 mL) was added TFA (7 mL). The reaction mixture was stirred at r.t for 2 hrs, and the resulting mixture was concentrated in vacuum. The residue was treated with sat. aq. NaHCO₃ to PH=7-8, then extracted with DCM:MeOH (10:1) (100 mL×3). The combined organic phase was washed with brine (80 mL), dried over Na₂SO₄, filtered and concentrated in vacuum to afford (7¹R,7³S,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one (700 mg, 83.3%). [M+H]⁺=527.

### Intermediate 3: (7¹R,7³S,E)-5⁶-((S)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

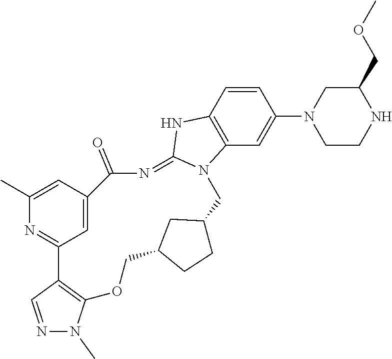

The title compound was prepared in a manner similar to that in Intermediate 2. [M+H]⁺=571.5

### Intermediate 4: (7¹R,7³S,E)-1¹,2⁶-dimethyl-5⁶-(2-oxa-5,8-diazaspiro[3.5]nonan-5-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

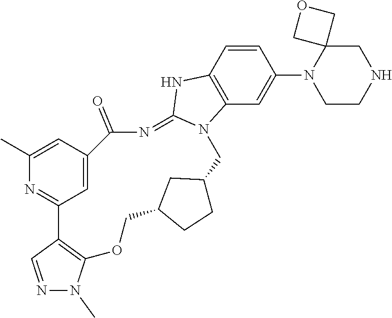 [SMILES: Cc1cc2cc(n1)-c1cnn(C)c1OC[C@H]1CC[C@H](C1)CN1/C(=N/C2=O)Nc2ccc(N3CCNCC34COC4)cc21]

The title compound was prepared in a manner similar to that in Intermediate 2. [M+H]⁺=569.5

### Intermediate 5: (7 S,7³R,E)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

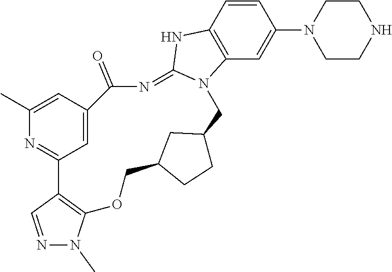 [SMILES: Cc1cc2cc(n1)-c1cnn(C)c1OC[C@@H]1CC[C@@H](C1)CN1/C(=N/C2=O)Nc2ccc(N3CCNCC3)cc21]

The title compound was prepared in a manner similar to that in Intermediates 1 and 2. [M+H]⁺=527.5

### Intermediate 6: 2,6-bis(benzyloxy)-3-(4-bromo-2,6-difluorophenyl)pyridine

### Step 1: 2,6-bis(benzyloxy)-3-(4,4,5,5-tetramethyl-1,3,2-dioxaborolan-2-yl)pyridine

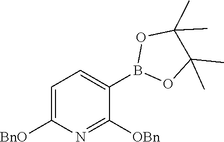 [SMILES: CC1(C)OB(c2ccc(OCc3ccccc3)nc2OCc2ccccc2)OC1(C)C]

4 reactions were carried out in parallel.

To a solution of 2,6-dibenzyloxy-3-bromo-pyridine (2.00 kg, 5.40 mol, 1.00 eq) in dioxane (12.0 L) was added 4,4,5,5-tetramethyl-1,3,2-dioxaborolane (760 g, 5.94 mol, 862 mL, 1.10 eq) and TEA (1.09 kg, 10.8 mol, 1.50 L, 2.00 eq), degassed and purged with N₂, then Pd(PPh₃)₂Cl₂ (189 g, 270 mmol, 0.05 eq) was added to the mixture. The mixture was stirred at 90° C. for 16 hrs. The reaction mixture was filtered and concentrated. 4 reactions were combined. The residue was purified by column chromatography (SiO₂, Petroleum ether/Ethyl acetate=1/0 to 0/1). 2,6-bis(benzyloxy)-3-(4,4,5,5-tetramethyl-1,3,2-dioxaborolan-2-yl)pyridine (5.80 kg, 13.9 mol, 64.3% yield) was obtained. [M+H]⁺=418.3

### Step 2: 5-bromo-1,3-difluoro-2-iodobenzene

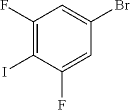

5 reactions were carried out in parallel.

To a solution of 1-bromo-3,5-difluoro-benzene (1.00 kg, 5.18 mol, 595 mL) in THF (4.00 L) was added LDA (2 M, 2.59 L) at −70° C. The mixture was stirred at −70° C. for 1 hr. Then to the mixture was added solution of I₂ (1.33 kg, 5.23 mol, 1.05 L) in THF (1.00 L) at −70° C. The mixture was stirred at −70° C. for 1 hr. The reaction mixture was poured into H₂O (5.00 L), extracted with EtOAc (3.00 L×2), 5 reactions were combined, the combined organic layers were washed by brine (5.00 L), dried over Na₂SO₄, filtered and concentrated in vacuum at 40° C. to give a residue. The crude product was triturated with petroleum ether (6.00 L). Compound 5-bromo-1,3-difluoro-2-iodobenzene (4.50 kg, 14.1 mol, 54.4% yield) was obtained. [M+H]⁺=318.8.

### Step 3: 2,6-bis(benzyloxy)-3-(4-bromo-2,6-difluorophenyl)pyridine

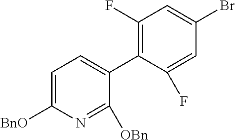 [SMILES: Fc1cc(Br)cc(F)c1-c1ccc(OCc2ccccc2)nc1OCc1ccccc1]

To a solution of 5-bromo-1,3-difluoro-2-iodobenzene (4.50 kg, 14.1 mol) and 2,6-bis(benzyloxy)-3-(4,4,5,5-tetramethyl-1,3,2-dioxaborolan-2-yl)pyridine (5.89 kg, 14.1 mol) in dioxane (22.5 L) and H₂O (4.50 L) was added Pd(PPh₃)₄ (1.63 kg, 1.41 mol), K₃PO₄ (8.99 kg, 42.3 mol). The mixture was stirred at 90° C. for 12 hrs. The mixture was filtered and filtrate was extracted with EtOAc (5.00 L), the combined organic was washed with brine (5.00 L), dried over Na₂SO₄, concentrated in vacuum. The residue was purified by column chromatography (SiO₂, Petroleum ether:Ethyl acetate=1:0 to 1:1). Compound 2,6-bis(benzyloxy)-3-(4-bromo-2,6-difluorophenyl)pyridine (3.00 kg, 6.01 mol, 42.5% yield, 96.5% purity) was obtained. ¹H NMR (400 MHz, CHLOROFORM-d) 7.47-7.40 (m, 1H), 7.39-7.34 (m, 2H), 7.34-7.29 (m, 2H), 7.29-7.25 (m, 4H), 7.25-7.17 (m, 2H), 7.12-7.05 (m, 2H), 6.42 (d, J=8.0 Hz, 1H), 5.32 (s, 2H), 5.28 (s, 2H); [M+H]⁺=482.1.

### Intermediate 7: (R)-3-(2,6-difluoro-4-(4-oxopiperidin-1-yl)phenyl)piperidine-2,6-dione

### Step 1: 8-(4-(2,6-bis(benzyloxy)pyridin-3-yl)-3,5-difluorophenyl)-1,4-dioxa-8-azaspiro[4.5]decane

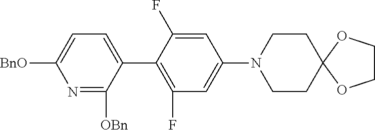 [SMILES: Fc1cc(N2CCC3(CC2)OCCO3)cc(F)c1-c1ccc(OCc2ccccc2)nc1OCc1ccccc1]

To the solution of intermediate 6 (30 g, 62.24 mmol), 1,4-dioxa-8-azaspiro[4.5]decane (10.68 g, 74.69 mmol) and Cs₂CO₃ (40.58 g, 124.48 mmol) in 500 mL dioxane, Pd₂(dba)₃ (2.85 g, 3.11 mmol) and Xantphos (3.6 g, 6.22 mmol) was added at N₂ atmosphere. The mixture was stirred at 80° C. for 16 hours under N₂ protected. The mixture was diluted with EtOAc and filtered. The filtrated was concentrated in vacuum and purified by silica column chromatography (EA:PE=0-80%) to afford the crude product. The crude was recrystallized with MeOH and filtered. The filter cake was dried to afford the title compound (26.8 g, 79% yield); [M+H]⁺=544.9.

### Step 2: 3-(2,6-difluoro-4-(1,4-dioxa-8-azaspiro[4.5]decan-8-yl)phenyl)piperidine-2,6-dione

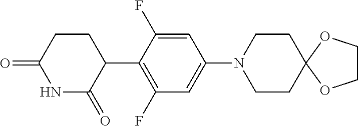 [SMILES: O=C1CCC(c2c(F)cc(N3CCC4(CC3)OCCO4)cc2F)C(=O)N1]

To the solution of 8-(4-(2,6-bis(benzyloxy)pyridin-3-yl)-3,5-difluorophenyl)-1,4-dioxa-8-azaspiro[4.5]decane (26.8 g, 49.26 mmol) in 400 mL DMF and 80 mL iPrOH, Pd/C (27 g, 10 wt. %, wet) was added. The mixture was stirred at 45° C. for 16 hours at H₂ atmosphere (4 bar). The mixture was filtered and the filter cake was washed with DMF. The combined liquid was concentrated in vacuum to afford the title compound (15 g, 83.2% yield); [M+H]⁺=367.1.

### Step 3: (R)-3-(2,6-difluoro-4-(1,4-dioxa-8-azaspiro[4.5]decan-8-yl)phenyl)piperidine-2,6-dione

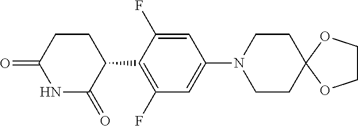 [SMILES: O=C1CC[C@H](c2c(F)cc(N3CCC4(CC3)OCCO4)cc2F)C(=O)N1]

The title compound was purified by chiral HPLC (CHIRALPAK IF (2*25 cm, 5 um), MtBE (0.1% DEA):(MeOH:DCM=1:1)=50:50, 100 bar, 20 ml/min) and the title compound corresponded to peak A @ 0.990 min/254 nm (4.47 g, 37% yield from 12 g racemate); [M+H]⁺=367.1.

### Step 4: (R)-3-(2,6-difluoro-4-(4-oxopiperidin-1-yl)phenyl)piperidine-2,6-dione

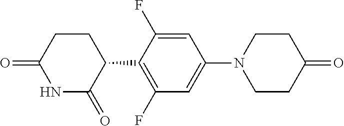 [SMILES: O=C1CCN(c2cc(F)c([C@H]3CCC(=O)NC3=O)c(F)c2)CC1]

(R)-3-(2,6-difluoro-4-(1,4-dioxa-8-azaspiro[4.5]decan-8-yl)phenyl)piperidine-2,6-dione (1 g, 2.73 mmol) was placed in 100 mL round bottom flask with a magnetic stir bar. Then, 10 mL 8 N HCl aqueous was added. The mixture was stirred at room temperature for 30 minutes. The mixture was added dropwise to sat. aq. NaHCO₃ solution and finally pH=6-7. The liquid was extracted with DCM and separated. The organic phase was concentrated in vacuum and purified with silica gel column chromatography (MeOH:DCM=0-5%) to afford the title compound (850 mg, 96.7% yield); [M+H]⁺=323.1.

### Intermediate 9: 3-(4-((3S,4R)-3-fluoro-4-(piperazin-1-yl)piperidin-1-yl)-3,3-dimethyl-2-oxoindolin-1-yl)piperidine-2,6-dione

### Step 1: 4-((3S,4R)-4-(4-benzylpiperazin-1-yl)-3-fluoropiperidin-1-yl)-1-(2,6-bis(benzyloxy)pyridin-3-yl)indolin-2-one

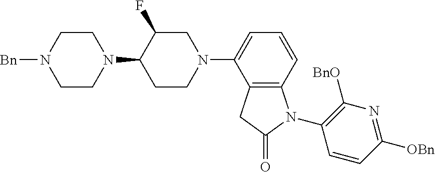 [SMILES: O=C1Cc2c(N3CC[C@@H](N4CCN(Cc5ccccc5)CC4)[C@@H](F)C3)cccc2N1c1ccc(OCc2ccccc2)nc1OCc1ccccc1]

The solution of 1-(2,6-bis(benzyloxy)pyridin-3-yl)-4-bromoindolin-2-one (1 g, 2 mmol, see step 2 in intermediate 10 synthesis), 1-benzyl-4-((3S,4R)-3-fluoropiperidin-4-yl)piperazine (831 mg, 3 mmol, prepared in a manner similar to that in intermediate 20), ^{t-}BuONa (576 mg, 6 mmol), Pd-PEPPSI-IPentCl (195 mg, 0.2 mmol) in DMA (15 mL) was degassed under reduced pressure and purged with N₂ for five times, and stirred under N₂ at 90° C. for 1 hour. After cooled to rt, the mixture was diluted with DCM (100 mL), then filtered through a pad of celite. The filtrate was washed with brine (150 mL), dried over anhydrous Na₂SO₄, concentrated under reduced pressure. The residue was purified by silica column chromatography (EA/PE, 40%) to afford product (810 mg, 60%). [M+H]⁺=698.3.

### Step 2: 4-((3S,4R)-4-(4-benzylpiperazin-1-yl)-3-fluoropiperidin-1-yl)-1-(2,6-bis(benzyloxy)pyridin-3-yl)-3,3-dimethylindolin-2-one

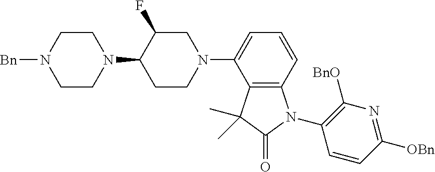 [SMILES: CC1(C)C(=O)N(c2ccc(OCc3ccccc3)nc2OCc2ccccc2)c2cccc(N3CC[C@@H](N4CCN(Cc5ccccc5)CC4)[C@@H](F)C3)c21]

4-((3S,4R)-4-(4-benzylpiperazin-1-yl)-3-fluoropiperidin-1-yl)-1-(2,6-bis(benzyloxy)pyridin-3-yl)indolin-2-one (697 mg, 1 mmol) in DMF (10 mL) and stirred under N₂ at 0° C. NaH (100 mg, 2.5 mmol, 60% in oil) added at this temperature. After 30 min CH₃I (355 mg, 2.5 mmol) was added and stirred under N₂ at room temperature for 1 hour. To this reaction solution was quenched with 20 mL water, it was extracted with DCM. The organic layer was washed with brine, saturated aqueous sodium thiosulfate and water, dried over anhydrous sodium sulfate. It was filtered and the resulting residue was concentrated, it was purified by using column chromatography (EA/PE, 40%) to afford product (500 mg, 69%). [M+H]⁺=726.5.

### Step 3: tert-butyl 4-((3S,4R)-1-(1-(2,6-dioxopiperidin-3-yl)-3,3-dimethyl-2-oxoindolin-4-yl)-3-fluoropiperidin-4-yl)piperazine-1-carboxylate

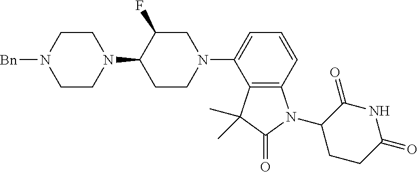 [SMILES: CC1(C)C(=O)N(C2CCC(=O)NC2=O)c2cccc(N3CC[C@@H](N4CCN(Cc5ccccc5)CC4)[C@@H](F)C3)c21]

Under N₂, to a solution of 4-((3S,4R)-4-(4-benzylpiperazin-1-yl)-3-fluoropiperidin-1-yl)-1-(2,6-bis(benzyloxy)pyridin-3-yl)-3,3-dimethylindolin-2-one (726 mg, 1 mmol) in DMF/ⁱ⁻PrOH (5 mL/5 mL) was added 10% Pd/C (100 mg) and (Boc)₂O at room temperature. And then the mixture was exchanged with H₂ two times and stirred under H₂ atmosphere at 50° C. for 15 hours. The mixture was filtered through a pad of Celite and washed with MeOH (50 mL). The filtrate was concentrated under vacuum to obtain the product (500 mg, 90%); [M+H]⁺=558.5.

### Step 4: 3-(4-((3S,4R)-3-fluoro-4-(piperazin-1-yl)piperidin-1-yl)-3,3-dimethyl-2-oxoindolin-1-yl)piperidine-2,6-dione

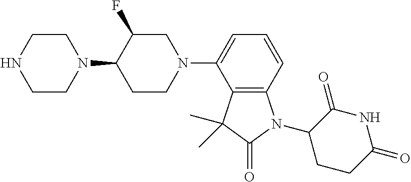 [SMILES: CC1(C)C(=O)N(C2CCC(=O)NC2=O)c2cccc(N3CC[C@@H](N4CCNCC4)[C@@H](F)C3)c21]

To a solution of tert-butyl 4-((3S,4R)-1-(1-(2,6-dioxopiperidin-3-yl)-3,3-dimethyl-2-oxoindolin-4-yl)-3-fluoropiperidin-4-yl)piperazine-1-carboxylate (279 mg, 0.5 mmol) in DCM (3 mL) was added TFA (1 mL). The reaction was stirred at room temperature for 2 hours and then concentrated in vacuo. The residue was dissolved in DCM (20 mL), washed with sat. NaHCO₃ solution (3×20 mL) and brine (50 mL), dried over Na₂SO₄, filtered and concentrated under reduced pressure to afford the product (180 mg, 78.6%); [M+H]⁺=458.5.

### Intermediate 10: 3-(3,3-dimethyl-2-oxo-4-(4-oxopiperidin-1-yl)indolin-1-yl)piperidine-2,6-dione

### Step 1: N-(2,6-bis(benzyloxy)pyridin-3-yl)-2-(2,6-dibromophenyl)acetamide

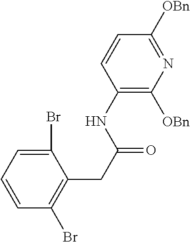 [SMILES: O=C(Cc1c(Br)cccc1Br)Nc1ccc(OCc2ccccc2)nc1OCc1ccccc1]

To a solution of 2-(2,6-dibromophenyl)acetic acid (10 g, 34.2 mmol), 2,6-bis(benzyloxy)pyridin-3-amine (11.5 g, 37.6 mml), DIEA (13.3 g, 102.6 mmol) in DMF (200 ml), were added HATU (19.5 g, 51.3 mmol) at 0° C. The mixture was stirred at RT overnight. The mixture was diluted with EA (500 mL) and then get solid filter. The mixture was concentrated under reduced pressure to afford crude product (15 g, 75.7%). [M+H]⁺=581.

### Step 2: 1-(2,6-bis(benzyloxy)pyridin-3-yl)-4-bromoindolin-2-one

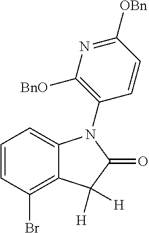 [SMILES: O=C1Cc2c(Br)cccc2N1c1ccc(OCc2ccccc2)nc1OCc1ccccc1]

A 500 mL round bottom flask equipped with a magnetic stirrer, were charged with N-(2,6-bis(benzyloxy)pyridin-3-yl)-2-(2,6-dibromophenyl)acetamide (15 g, 25.9 mmol), ((2-bromoethoxy)methyl)benzene (15.4 g, 71.5 mmol), K₂CO₃ (17.8 g, 129.5 mmol), CuCl (2.56 g, 25.9 mmol), pentane-2,4-dione (5.17 g, 51.8 mmol) and NMP (200 mL). The mixture was degassed under vacuum and purged with N₂ for three times. The resulting mixture was stirred for 3 hours at 85° C. under N₂. After cooled to rt, the mixture was diluted with ethyl acetate (300 mL), then filtered through a pad of celite. The filtrate was washed with brine (300 mL), dried over anhydrous Na₂SO₄, concentrated under reduced pressure. The residue was purified by silica column chromatography (EA/PE, 15%) to afford product (8.00 g, 61.8%). [M+H]⁺=501.

### Step 3:1-(2,6-bis(benzyloxy)pyridin-3-yl)-4-(1,4-dioxa-8-azaspiro[4.5]decan-8-yl)indolin-2-one

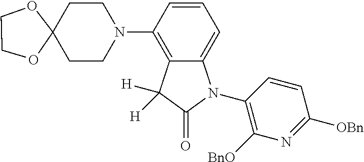 [SMILES: O=C1Cc2c(N3CCC4(CC3)OCCO4)cccc2N1c1ccc(OCc2ccccc2)nc1OCc1ccccc1]

The solution of 1-(2,6-bis(benzyloxy)pyridin-3-yl)-4-bromoindolin-2-one (8 g, 15.9 mmol), 1,4-dioxa-8-azaspiro[4.5]decane (3.43 g, 23.9 mmol), Cs₂CO₃ (15.62 g, 47.7 mmol), Pd-PEPPSI-IPentCl (0.67 g, 0.80 mmol) in dioxane (120 mL) was degassed under reduced pressure and purged with N₂ for five times, and stirred under N₂ at 100° C. overnight. After cooled to rt, the mixture was diluted with ethyl acetate (100 mL), then filtered through a pad of celite. The filtrate was washed with brine (150 mL), dried over anhydrous Na₂SO₄, concentrated under reduced pressure. The residue was purified by silica column chromatography (EA/PE, 40%) to afford product (2 g, 22.2%). [M+H]⁺=564.

### Step 4: 1-(2,6-bis(benzyloxy)pyridin-3-yl)-3,3-dimethyl-4-(1,4-dioxa-8-azaspiro[4.5]decan-8-yl)indolin-2-one

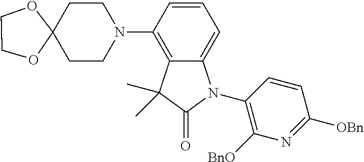 [SMILES: CC1(C)C(=O)N(c2ccc(OCc3ccccc3)nc2OCc2ccccc2)c2cccc(N3CCC4(CC3)OCCO4)c21]

The solution of 1-(2,6-bis(benzyloxy)pyridin-3-yl)-4-(1,4-dioxa-8-azaspiro[4.5]decan-8-yl)indolin-2-one (2 g, 3.55 mmol) and NaH (0.71 g, 17.75 mmol) in DMF (20 mL) was stirred at 0° C. for 30 min. Then CH₃I (2.5 g, 17.75 mmol) was added dropwise into the mixture while maintaining temperature at 0° C. The resulting mixture was stirred at RT for 4 hrs. The mixture was poured into EA (20 mL), and washed with brine (20 mL), water (3′20 mL), brine (20 mL) in turn, then dried over anhydrous Na₂SO₄, concentrated in vacuum. The residue was purified by silica column chromatography (EA/PE, 15%) to afford product (1.5 g, 71.4%). [M+H]⁺=592.

### Step 5: 3-(3,3-dimethyl-2-oxo-4-(1,4-dioxa-8-azaspiro[4.5]decan-8-yl)indolin-1-yl)piperidine-2,6-dione

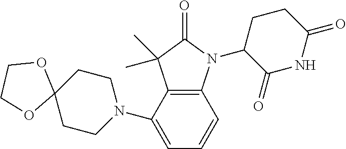 [SMILES: CC1(C)C(=O)N(C2CCC(=O)NC2=O)c2cccc(N3CCC4(CC3)OCCO4)c21]

A 100 mL round bottom flask equipped with a magnetic stirrer were charged with 1-(2,6-bis(benzyloxy)pyridin-3-yl)-3,3-dimethyl-4-(1,4-dioxa-8-azaspiro[4.5]decan-8-yl)indolin-2-one (1.5 g, 2.53 mmol), DCM/ETOH (10 mL/20 mL), and Pd/C (10 wt %, 1.5 g). The resulting mixture was degassed under reduced pressure and purged with H₂ for five times, then stirred at RT for overnight. The mixture was diluted with DCM/MEOH (50 mL/50 mL), then sonicated in an ultrasonic washer for 5 minutes, followed by filtration through a pad of celite. The filtrate was concentrated under vacuum. The residue was purified by silica column chromatography (PE/EA=1:1) to afford product (560.1 mg, 53.6%). [M+H]⁺=414.

¹H NMR (300 MHz, DMSO) δ 11.05 (s, 1H), 7.22 (t, J=7.4 Hz, 1H), 7.03 (d, J=8.2 Hz, 1H), 6.81 (d, J=7.7 Hz, 1H), 5.21 (s, 1H), 3.93 (s, 4H), 3.32 (s, 2H), 2.87 (t, J=5.5 Hz, 5H), 1.94 (s, 1H), 1.86-1.71 (m, 4H), 1.40 (s, 6H).

### Step 6: 3-(3,3-dimethyl-2-oxo-4-(4-oxopiperidin-1-yl)indolin-1-yl)piperidine-2,6-dione

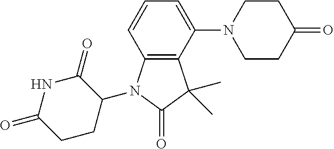 [SMILES: CC1(C)C(=O)N(C2CCC(=O)NC2=O)c2cccc(N3CCC(=O)CC3)c21]

The compound was prepared in a procedure similar to that in intermediate 7 step 4.

### Intermediate 11: 2′,6′-bis(benzyloxy)-3-fluoro-5-iodo-6-methyl-2,3′-bipyridine

### Step 1: 5-fluoro-2-methylpyridin-3-amine

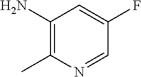

To a solution of 2-bromo-5-fluoropyridin-3-amine (20 g, 105.26 mmol) in dioxane/H₂O (200/40 mL) were added 2,4,6-trimethyl-1,3,5,2,4,6-trioxatriborinane (15.91 g, 126.32 mmol), Pd(dppf)Cl₂ (8.59 g, 10.53 mmol), and K₂CO₃ (43.57 g, 315.79 mmol). The resulting solution was stirred overnight at 120° C. under N₂ atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with MeOH/DCM (0˜10%) to afford 5-fluoro-2-methylpyridin-3-amine (12 g, 90.1%). [M+H]⁺=127.1

### Step 2: 6-chloro-5-fluoro-2-methylpyridin-3-amine

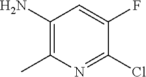

To a stirred mixture of 5-fluoro-2-methylpyridin-3-amine (6 g, 47.62 mmol) in DMF (120 mL) were added NCS (8.43 g, 47.62 mmol) at 0° C. The mixture was stirred overnight at 60° C. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with EA/PE (0˜50%) to afford 6-chloro-5-fluoro-2-methylpyridin-3-amine (3 g, 39.5%). [M+H]⁺=161.0.

### Step 3: 2′,6′-bis(benzyloxy)-3-fluoro-6-methyl-[2,3′-bipyridin]-5-amine

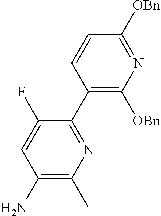 [SMILES: Cc1nc(-c2ccc(OCc3ccccc3)nc2OCc2ccccc2)c(F)cc1N]

To a solution of 6-chloro-5-fluoro-2-methylpyridin-3-amine (3 g, 18.63 mmol) in dioxane/H₂O (30/5 mL) were added 2,6-bis(benzyloxy)-3-(4,4,5,5-tetramethyl-1,3,2-dioxaborolan-2-yl)pyridine (9.32 g, 22.36 mmol), Pd(dppf)Cl₂ (1.52 g, 1.86 mmol), and K₂CO₃ (7.71 g, 55.89 mmol). The resulting solution was stirred overnight at 100° C. under N₂ atmosphere. After cooling to rt, diluted with H₂O and extracted with EtOAc. The combined organic layer was washed with brine, dried by Na₂SO₄ and concentrated. The filtrate was concentrated under reduced pressure. The crude product was purified by silica column chromatography (EA/PE=0-50%) to afford 2′,6′-bis(benzyloxy)-3-fluoro-6-methyl-[2,3′-bipyridin]-5-amine (5 g, 64.3%). [M+H]⁺=416.2

### Step 4: 2′,6′-bis(benzyloxy)-3-fluoro-5-iodo-6-methyl-2,3′-bipyridine

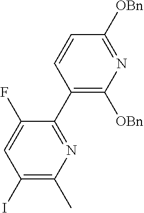 [SMILES: Cc1nc(-c2ccc(OCc3ccccc3)nc2OCc2ccccc2)c(F)cc1I]

To a solution of 2′,6′-bis(benzyloxy)-3-fluoro-6-methyl-[2,3′-bipyridin]-5-amine (3 g, 7.23 mmol) in ACN (30 mL) were added KI (6 g, 36.14 mmol), CuI (1.65 g, 8.67 mmol), and t-BuONO (3.72 g, 36.14 mmol). The resulting solution was stirred overnight at 80° C. under N₂ atmosphere. After cooling to rt, the mixture was diluted with H₂O and extracted with EtOAc. The combined organic layer was washed with brine, dried by Na₂SO₄ and concentrated. The filtrate was concentrated under reduced pressure. The crude product purified by silica column chromatography (EA/PE=0-15%) to afford 2′,6′-bis(benzyloxy)-3-fluoro-5-iodo-6-methyl-2,3′-bipyridine (2.5 g, 65.79%). [M+H]⁺=527.1

### Intermediate 12: 3-(3-fluoro-6-methyl-5-(4-oxopiperidin-1-yl)pyridin-2-yl)piperidine-2,6-dione

### Step 1: 8-(2′,6′-bis(benzyloxy)-3-fluoro-6-methyl-[2,3′-bipyridin]-5-yl)-1,4-dioxa-8-azaspiro[4.5]decane

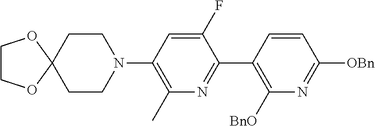 [SMILES: Cc1nc(-c2ccc(OCc3ccccc3)nc2OCc2ccccc2)c(F)cc1N1CCC2(CC1)OCCO2]

A mixture of intermediate 11 (5.2 g, 9.89 mmol), 1,4-dioxa-8-azaspiro[4.5]decane (2.11 g, 14.83 mmol), Ruphos (0.94 g, 0.20 mmol), Pd₂(dba)₃ (0.9 g, 0.99 mol), and Cs₂CO₃ (9.7 g, 29.66 mmol) in 1,4-dioxane (50 mL) was stirred for 8 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product was purified by silica column chromatography (EA/PE=0-30%) to afford 8-(2′,6′-bis(benzyloxy)-3-fluoro-6-methyl-[2,3′-bipyridin]-5-yl)-1,4-dioxa-8-azaspiro[4.5]decane (4.5 g, 85.0%). [M+H]⁺=542.0

### Step 2: 3-(3-fluoro-6-methyl-5-(1,4-dioxa-8-azaspiro[4.5]decan-8-yl)pyridin-2-yl)piperidine-2,6-dione

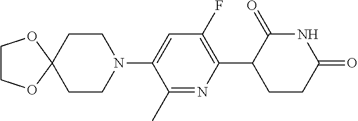 [SMILES: Cc1nc(C2CCC(=O)NC2=O)c(F)cc1N1CCC2(CC1)OCCO2]

To a stirred solution of 8-(2′,6′-bis(benzyloxy)-3-fluoro-6-methyl-[2,3′-bipyridin]-5-yl)-1,4-dioxa-8-azaspiro[4.5]decane (4.5 g, 8.31 mmol) in THF (100 mL) was added Pd/C (10 wt %, 4.5 g). The resulting mixture was degassed under reduced pressure and purged with H₂ for five times, then stirred overnight at rt. The resulting mixture was filtered, and the filter cake was washed with THF. The filtrate was concentrated under reduced pressure. And the residue was purified by column chromatography (EA/PE, 30-50%) to afford 3-(3-fluoro-6-methyl-5-(1,4-dioxa-8-azaspiro[4.5]decan-8-yl)pyridin-2-yl)piperidine-2,6-dione (3.53 g, 9.8 mmol, 83.0%).

¹H NMR (300 MHz, DMSO) δ 10.86 (s, 1H), 7.35 (d, J=12 Hz, 1H), 4.13 (m, 1H), 3.32 (s, 1H), 2.94 (t, J=6 Hz, 4H), 2.71 (m, 1H), 2.62-2.50 (m, 1H), 2.37 (d, J=3 Hz, 3H), 2.26-2.15 (m, 1H), 2.03 (m, 1H), 1.78 (t, J=6 Hz, 4H), 1.36 (s, 1H), 1.22 (d, J=12 Hz, 1H), 0.99-0.80 (m, 1H).

### Step 3: 3-(3-fluoro-6-methyl-5-(4-oxopiperidin-1-yl)pyridin-2-yl)piperidine-2,6-dione

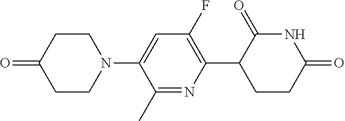 [SMILES: Cc1nc(C2CCC(=O)NC2=O)c(F)cc1N1CCC(=O)CC1]

The compound was prepared in a procedure similar to that in intermediate 7 step 4.

### Intermediate 13: 3-(4,6-dimethyl-5-(4-oxopiperidin-1-yl)pyridin-2-yl)piperidine-2,6-dione

### Step 1:2′,6′-bis(benzyloxy)-5-bromo-4,6-dimethyl-2,3′-bipyridine

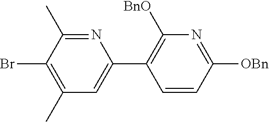 [SMILES: Cc1cc(-c2ccc(OCc3ccccc3)nc2OCc2ccccc2)nc(C)c1Br]

To a solution of 3-bromo-6-chloro-2,4-dimethylpyridine (9 g, 40.91 mmol) in dioxane/H₂O (100/20 mL) were added 2,6-bis(benzyloxy)-3-(4,4,5,5-tetramethyl-1,3,2-dioxaborolan-2-yl)pyridine (17.1 g, 40.91 mmol), Pd(PPh₃)₄ (4.64 g, 4.10 mmol), and K₂CO₃ (16.94 g, 122.73 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-50%) to afford 2′,6′-bis(benzyloxy)-5-bromo-4,6-dimethyl-2,3′-bipyridine (12 g, 62.2%). [M+H]⁺=475.1

### Step 2: 8-(2′,6′-bis(benzyloxy)-4,6-dimethyl-[2,3′-bipyridin]-5-yl)-1,4-dioxa-8-azaspiro[4.5]decane

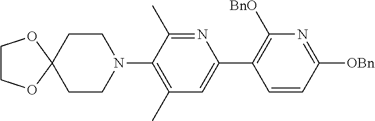 [SMILES: Cc1cc(-c2ccc(OCc3ccccc3)nc2OCc2ccccc2)nc(C)c1N1CCC2(CC1)OCCO2]

To a stirred mixture of 2′,6′-bis(benzyloxy)-5-bromo-4,6-dimethyl-2,3′-bipyridine (2.3 g, 4.84 mmol), 1,4-dioxa-8-azaspiro[4.5]decane (1.0 g, 7.26 mmol), and Cs₂CO₃ (4.7 g, 14.52 mmol) in DMF (20 mL) was added Pd-PEPPSI-IPentCl (406.7 mg, 0.48 mmol). The resulting mixture was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-30%) to afford 8-(2′,6′-bis(benzyloxy)-4,6-dimethyl-[2,3′-bipyridin]-5-yl)-1,4-dioxa-8-azaspiro[4.5]decane (1.5 g, 57.7%). [M+H]+=538.3

### Step 3: 3-(4,6-dimethyl-5-(1,4-dioxa-8-azaspiro[4.5]decan-8-yl)pyridin-2-yl)piperidine-2,6-dione

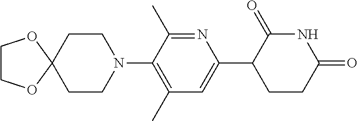 [SMILES: Cc1cc(C2CCC(=O)NC2=O)nc(C)c1N1CCC2(CC1)OCCO2]

To a stirred solution of 8-(2′,6′-bis(benzyloxy)-4,6-dimethyl-[2,3′-bipyridin]-5-yl)-1,4-dioxa-8-azaspiro[4.5]decane (3.4 g, 8.31 mmol) in THF (50 mL) was added Pd/C (10 wt %, 3.5 g). The resulting mixture was degassed under reduced pressure and purged with H₂ for five times, then stirred overnight at rt. The resulting mixture was filtered, the filter cake was washed with THF. The filtrate was concentrated under reduced pressure. And the residue was purified by column chromatography (EA/PE=30-50%) to afford 3-(4,6-dimethyl-5-(1,4-dioxa-8-azaspiro[4.5]decan-8-yl)pyridin-2-yl)piperidine-2,6-dione (2.09 g, 83.0%). [M+H]⁺=360.2. ¹H NMR (300 MHz, DMSO) δ 10.77 (s, 1H), 6.96 (s, 1H), 3.92 (s, 4H), 3.83 (m, 1H), 3.03 (t, J=6 Hz, 4H), 2.62-2.51 (m, 2H), 2.39 (s, 3H), 2.26 (s, 3H), 2.18 (m, 1H), 2.12-2.00 (m, 1H), 1.72 (t, J=6 Hz, 4H).

### Step 4: 3-(4,6-dimethyl-5-(4-oxopiperidin-1-yl)pyridin-2-yl)piperidine-2,6-dione

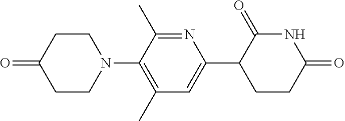 [SMILES: Cc1cc(C2CCC(=O)NC2=O)nc(C)c1N1CCC(=O)CC1]

The compound was prepared in a procedure similar to that in intermediate 7 step 4.

### Intermediate 14: 3-(4-methyl-5-(4-oxopiperidin-1-yl)pyridin-2-yl)piperidine-2,6-dione

### Step 1: 2′,6′-bis(benzyloxy)-5-bromo-4-methyl-2,3′-bipyridine

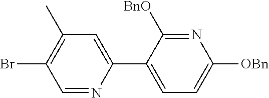 [SMILES: Cc1cc(-c2ccc(OCc3ccccc3)nc2OCc2ccccc2)ncc1Br]

To the solution of 5-bromo-2-iodo-4-methylpyridine (4.8 g, 16.1 mmol), 2,6-bis(benzyloxy)-3-(4,4,5,5-tetramethyl-1,3,2-dioxaborolan-2-yl)pyridine (6.7 g, 16.1 mmol) and K₂CO₃ (4.5 g, 32.2 mmol) in 100 mL 1,4-dioxane and 20 mL H₂O was added Pd(dppf)Cl₂ (1.2 g, 1.61 mmol). The mixture was stirred at 90° C. for 16 hours. The mixture was concentrated under reduced pressure to give the crude residue, which was purified by silica column chromatography (PE:EA=100:1-5:1) to afford the product (6.7 g, 90.5%). [M+H]⁺=461.5.

### Step 2: 8-(2′,6′-bis(benzyloxy)-4-methyl-[2,3′-bipyridin]-5-yl)-1,4-dioxa-8-azaspiro[4.5]decane

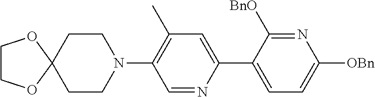 [SMILES: Cc1cc(-c2ccc(OCc3ccccc3)nc2OCc2ccccc2)ncc1N1CCC2(CC1)OCCO2]

To the solution of 2′,6′-bis(benzyloxy)-5-bromo-4-methyl-2,3′-bipyridine (6.7 g, 14.5 mmol), 1,4-dioxa-8-azaspiro[4.5]decane (5.2 g, 36.3 mmol) and Cs₂CO₃ (9.4 g, 29.0 mmol) in 80 mL DMA, were added Pd₂(dba)₃ (2.6 g, 2.9 mmol) and RuPhos (2.7 g, 5.8 mmol). The mixture was stirred at 100° C. for 16 hours under nitrogen atmosphere. The mixture was filtered through a celite pad and washed with DCM. The filtrate was concentrated under reduced pressure to give the crude residue, which was purified by silica column chromatography (PE:EA=50:1-2:1) to afford the product (5.3 g, 69.7%). [M+H]⁺=524.5.

### Step 3: 3-(4-methyl-5-(1,4-dioxa-8-azaspiro[4.5]decan-8-yl)pyridin-2-yl)piperidine-2,6-dione

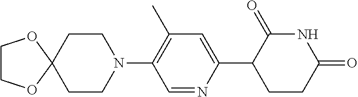 [SMILES: Cc1cc(C2CCC(=O)NC2=O)ncc1N1CCC2(CC1)OCCO2]

To the solution of 8-(2′,6′-bis(benzyloxy)-4-methyl-[2,3′-bipyridin]-5-yl)-1,4-dioxa-8-azaspiro[4.5]decane (5.3 g, 10.1 mmol) in 75 mL DMF and 75 mL iPrOH, Pd/C (2.0 g, 10 wt. %, wet) was added. The mixture was stirred at 50° C. for 20 hours under hydrogen atmosphere (balloon). The mixture was cooled to room temperature and filtered by celite directly. The filtrate was concentrated in vacuum to afford the desired product (2.7 g, 77.1%). [M+H]⁺=346.6.

### Step 4: 3-(4-methyl-5-(4-oxopiperidin-1-yl)pyridin-2-Yl)piperidine-2,6-dione

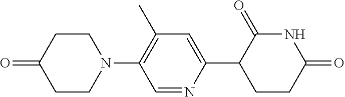 [SMILES: Cc1cc(C2CCC(=O)NC2=O)ncc1N1CCC(=O)CC1]

3-(4-methyl-5-(1,4-dioxa-8-azaspiro[4.5]decan-8-yl)pyridin-2-yl)piperidine-2,6-dione (2.7 g, 7.8 mmol) was placed in 250 mL round bottom flask with a magnetic stir bar. Then 45 mL 8 N HCl aqueous was added. The mixture was stirred at room temperature for 2 hours. The mixture was added dropwise to sat. aq. NaHCO₃ and the pH was adjusted to 6-7. The liquid was extracted with DCM (40 mL×3) and separated. The combined organic phase was concentrated in vacuum and purified with combiflash (DCM:MeOH=25:1) to afford the title compound (2.2 g, 93.6% yield). [M+H]⁺=302.5.

### Intermediate 15: 3-(6-methyl-5-(4-oxopiperidin-1-yl)pyridin-2-yl)piperidine-2,6-dione

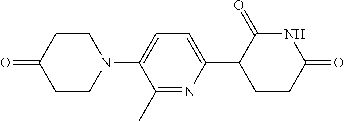 [SMILES: Cc1nc(C2CCC(=O)NC2=O)ccc1N1CCC(=O)CC1]

The title compound was prepared in a procedure similar to that in intermediate 14.

### Intermediate 16: 3-(4-ethyl-5-(4-oxopiperidin-1-yl)pyridin-2-yl)piperidine-2,6-dione

### Step 1: 5-bromo-2-chloro-4-vinylpyridine

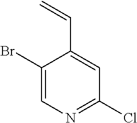

To a stirred mixture of 5-bromo-2-chloro-4-iodopyridine (16 g, 50.3 mmol), 4,4,5,5-tetramethyl-2-vinyl-1,3,2-dioxaborolane (7.75 g, 50.3 mmol), and Na₂CO₃ (16.00 g, 150.9 mmol) in 1,4-Dioxacyclohexane (200 mL) and H₂O (40 mL) was added Pd(dppf)Cl₂ (3.65 g, 5.03 mmol). The resulting mixture was degassed under reduced pressure and purged with N₂ for three times. Then it was stirred at 100° C. for 5 hours. The reaction mixture was diluted with water and extracted with EA. The combined organic layers were washed with brine, dried, and concentrated. The residue was purified by column chromatography (EA/PE=20-30%) to afford 5-bromo-2-chloro-4-vinylpyridine (6.8 g, 62.0%). [M+H]⁺=218.

### Step 2: 8-(6-chloro-4-vinylpyridin-3-yl)-1,4-dioxa-8-azaspiro[4.5]decane

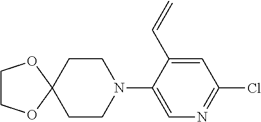 [SMILES: C=Cc1cc(Cl)ncc1N1CCC2(CC1)OCCO2]

To a stirred mixture of 5-bromo-2-chloro-4-vinylpyridine (6.8 g, 31.2 mmol), 1,4-dioxa-8-azaspiro[4.5]decane (4.46 g, 31.2 mmol), and Cs₂CO₃ (20.3 g, 62.4 mmol) in 1,4-dioxacyclohexane (140 mL) was added Pd-PEPPSI-IPentCl (1.3 g, 1.56 mmol). The resulting mixture was degassed under reduced pressure and purged with N₂ for three times. Then it was stirred overnight at 100° C. The reaction mixture was filtered through a celite pad, and the filtrate was concentrated. The residue was purified by column chromatography (EA/PE=26-28%) to afford 8-(6-chloro-4-vinylpyridin-3-yl)-1,4-dioxa-8-azaspiro[4.5]decane (4.2 g, 47.9%). [M+H]+=281.

### Step 3: 8-(2′,6′-bis(benzyloxy)-4-vinyl-[2,3′-bipyridin]-5-yl)-1,4-dioxa-8-azaspiro[4.5]decane

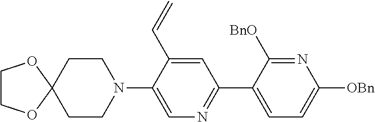 [SMILES: C=Cc1cc(-c2ccc(OCc3ccccc3)nc2OCc2ccccc2)ncc1N1CCC2(CC1)OCCO2]

To a stirred mixture of 8-(6-chloro-4-vinylpyridin-3-yl)-1,4-dioxa-8-azaspiro[4.5]decane (4.2 g, 14.95 mmol), 2,6-bis(benzyloxy)-3-(4,4,5,5-tetramethyl-1,3,2-dioxaborolan-2-yl)pyridine (6.23 g, 14.95 mmol), and K₂CO₃ (6.19 g, 44.85 mmol) in 1,4-Dioxacyclohexane (50 mL) and H₂O (10 mL) was added Pd(dppf)Cl₂ (1.09 g, 1.5 mmol). The resulting mixture was degassed under reduced pressure and purged with N₂ for three times. Then it was stirred at 100° C. for 5 hours. The reaction mixture was diluted with water and extracted with EA. The combined organic layers were washed with brine, dried, and concentrated. The residue was purified by column chromatography (EA/PE=20-30%) to afford 8-(2′,6′-bis(benzyloxy)-4-vinyl-[2,3′-bipyridin]-5-yl)-1,4-dioxa-8-azaspiro[4.5]decane (6.3 g, 78.9%). [M+H]⁺=536

### Step 4: 3-(4-ethyl-5-(1,4-dioxa-8-azaspiro[4.5]decan-8-yl)pyridin-2-yl)piperidine-2,6-dione

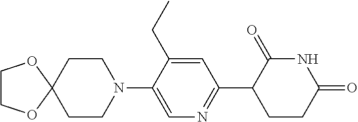 [SMILES: CCc1cc(C2CCC(=O)NC2=O)ncc1N1CCC2(CC1)OCCO2]

To a stirred solution of 8-(2′,6′-bis(benzyloxy)-4-vinyl-[2,3′-bipyridin]-5-yl)-1,4-dioxa-8-azaspiro[4.5]decane (6.3 g, 11.8 mmol) in THF (150 mL) was added Pd/C (10 wt %, 6.3 g). The resulting mixture was degassed under reduced pressure and purged with H₂ for five times, then stirred overnight at 50° C. The reaction mixture was diluted with THF/DCM (1:1), then filtered through a Celite pad. The filtrate was concentrated under vacuum. And the residue was purified by column chromatography (EA/PE, 30-50%) to afford 3-(4-ethyl-5-(1,4-dioxa-8-azaspiro[4.5]decan-8-yl)pyridin-2-yl)piperidine-2,6-dione. (3.53 g, 9.8 mmol, 83.05%). [M+H]+=360. ¹H NMR (300 MHz, DMSO) δ 10.80 (s, 1H), 8.19 (s, 1H), 7.20 (s, 1H), 3.99-3.90 (m, 1H), 3.32 (s, 1H), 2.96 (t, J=3 Hz, 4H), 2.72-2.50 (m, 3H), 2.26-2.04 (m, 1H), 1.76 (t, J=6 Hz, 4H), 1.20 (t, J=9 Hz, 3H), 1.07 (s, 2H).

### Step 5: 3-(4-ethyl-5-(4-oxopiperidin-1-yl)pyridin-2-yl)piperidine-2,6-dione

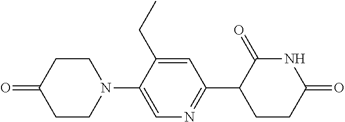 [SMILES: CCc1cc(C2CCC(=O)NC2=O)ncc1N1CCC(=O)CC1]

The compound was prepared in a procedure similar to that in intermediate 14 step 4.

### Intermediate 17: 3-(6-ethyl-5-(4-oxopiperidin-1-yl)pyridin-2-yl)piperidine-2,6-dione

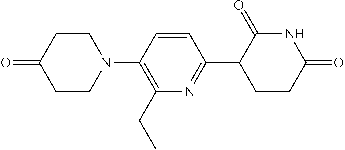 [SMILES: CCc1nc(C2CCC(=O)NC2=O)ccc1N1CCC(=O)CC1]

The compound was prepared in a procedure similar to that in intermediate 16.

### Intermediate 18: (1-(6-(2,6-dioxopiperidin-3-yl)-2,4-dimethylpyridin-3-yl)azetidin-3-yl)methyl methanesulfonate

### Step 1: 2′,6′-bis(benzyloxy)-5-(3-((benzyloxy)methyl)azetidin-1-yl)-4,6-dimethyl-2,3′-bipyridine

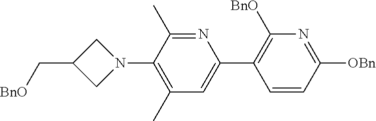 [SMILES: Cc1cc(-c2ccc(OCc3ccccc3)nc2OCc2ccccc2)nc(C)c1N1CC(COCc2ccccc2)C1]

The solution of 2′,6′-bis(benzyloxy)-5-bromo-4,6-dimethyl-2,3′-bipyridine (5 g, 10.5 mmol), 3-((benzyloxy)methyl)azetidine (2.81 g, 15.79 mml), Cs₂CO₃ (10.29 g, 31.59 mmol), Pd-PEPPSI-IPentCl (0.44 g, 0.53 mml) in DMF (60 ml) was degassed under reduced pressure and purged with N₂ for five times, and stirred under N₂ at 100° C. for 5 hrs. After cooled to rt, the mixture was poured into EA (100 mL), and washed with brine (100 mL), water (3′100 mL), brine (100 mL) in turn, then dried over anhydrous Na₂SO₄, concentrated in vacuum. The residue was purified by silica column chromatography PE/EA (6:1) to afford 2′,6′-bis(benzyloxy)-5-(3-((benzyloxy)methyl)azetidin-1-yl)-4,6-dimethyl-2,3′-bipyridine (3.7 g, 61.4%). [M+H]⁺=572.

### Step 2: 3-(5-(3-(hydroxymethyl)azetidin-1-yl)-4,6-dimethylpyridin-2-yl)piperidine-2,6-dione

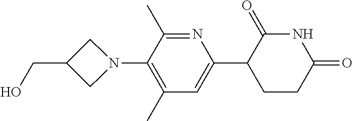 [SMILES: Cc1cc(C2CCC(=O)NC2=O)nc(C)c1N1CC(CO)C1]

A 250 mL round bottom flask equipped with a magnetic stirrer, were charged with 2′,6′-bis(benzyloxy)-5-(3-((benzyloxy)methyl)azetidin-1-yl)-4,6-dimethyl-2,3′-bipyridine (3.7 g, 6.49 mmol), THF (50 mL), and Pd/C (10 wt %, 3.7 g). The resulting mixture was degassed under reduced pressure and purged with H₂ for five times, then stirred at 50° C. for overnight. The mixture was diluted with THF (100 mL), then sonicated in an ultrasonic washer for 5 minutes, followed by filtration through a pad of celite. The filtrate was concentrated under vacuum. The residue was triturated with PE to afford 3-(5-(3-(hydroxymethyl)azetidin-1-yl)-4,6-dimethylpyridin-2-yl)piperidine-2,6-dione (1.03 g, 52.6%). [M+H]⁺=304.

### Step 3: (1-(6-(2,6-dioxopiperidin-3-yl)-2,4-dimethylpyridin-3-yl)azetidin-3-yl)methyl methanesulfonate

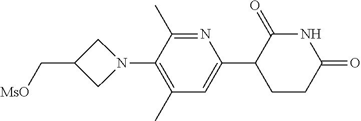 [SMILES: Cc1cc(C2CCC(=O)NC2=O)nc(C)c1N1CC(COS(C)(=O)=O)C1]

The compound was prepared in a manner similar to that in Intermediate 19 step 5.

### Intermediate 19: 2-(6-(2,6-dioxopiperidin-3-yl)-2,4-dimethylpyridin-3-yl)ethyl methanesulfonate

### Step 1: 2′,6′-bis(benzyloxy)-5-bromo-4,6-dimethyl-2,3′-bipyridine

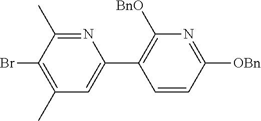 [SMILES: Cc1cc(-c2ccc(OCc3ccccc3)nc2OCc2ccccc2)nc(C)c1Br]

To a solution of 3-bromo-6-chloro-2,4-dimethylpyridine (9 g, 40.9 mmol) in dioxane/H₂O (100/20 mL) were added 2,6-bis(benzyloxy)-3-(4,4,5,5-tetramethyl-1,3,2-dioxaborolan-2-yl)pyridine (17.1 g, 40.9 mmol), Pd(PPh₃)₄ (4.64 g, 4.10 mmol), and K₂CO₃ (16.94 g, 122.73 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-50%) to afford 2′,6′-bis(benzyloxy)-5-bromo-4,6-dimethyl-2,3′-bipyridine (12 g, 62.2%). [M+H]⁺=475.1.

### Step 2: 2′,6′-bis(benzyloxy)-4,6-dimethyl-5-vinyl-2,3′-bipyridine

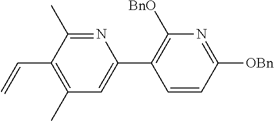 [SMILES: C=Cc1c(C)cc(-c2ccc(OCc3ccccc3)nc2OCc2ccccc2)nc1C]

To a stirred mixture of 2′,6′-bis(benzyloxy)-5-bromo-4,6-dimethyl-2,3′-bipyridine (3 g, 6.31 mmol), 4,4,5,5-tetramethyl-2-vinyl-1,3,2-dioxaborolane (1.17 g, 7.58 mmol), and K₂CO₃ (2.61 g, 18.93 mmol) in 1,4-dioxane (30 mL) and H₂O (6 mL) was added Pd(dppf)Cl₂ (514.1 mg, 0.63 mmol). The resulting solution was stirred for 5 h at 100° C. under N₂ atmosphere. After cooling to rt, diluted with H₂O and extracted with EtOAc, the combined organic layer was washed with brine, dried by Na₂SO₄ and concentrated. The filtrate was concentrated under reduced pressure. The crude product purified by silica column chromatography (EA/PE=0-20%) to afford 2′,6′-bis(benzyloxy)-4,6-dimethyl-5-vinyl-2,3′-bipyridine (1.7 g, 63.9%). [M+H]⁺=423.0

### Step 3: 2-(2′,6′-bis(benzyloxy)-4,6-dimethyl-[2,3′-bipyridin]-5-yl)ethan-1-ol

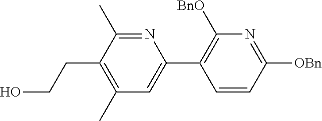 [SMILES: Cc1cc(-c2ccc(OCc3ccccc3)nc2OCc2ccccc2)nc(C)c1CCO]

To a stirred mixture of 2′,6′-bis(benzyloxy)-4,6-dimethyl-5-vinyl-2,3′-bipyridine (1.7 g, 4.01 mmol) in THF (20 mL) was added 9-BBN (0.5 M in THF, 40 mL, 20.0 mmol) dropwise at 0° C. The reaction mixture was stirred overnight at rt, then NaOH (2M in water, 4 mL, 8.03 mmol) and H₂O₂ (30%, 40.2 mL, 12.1 mmol) were added at 0° C. The reaction mixture was stirred at rt for 2 h, The mixture was diluted with H₂O and extracted with EA, and the residue was purified by column chromatography (EA/PE=0-35%) to afford 2-(2′,6′-bis(benzyloxy)-4,6-dimethyl-[2,3′-bipyridin]-5-yl)ethan-1-ol (1.4 g, 79.5%). [M+H]⁺=441.2

### Step 4: 3-(5-(2-hydroxyethyl)-4,6-dimethylpyridin-2-yl)piperidine-2,6-dione

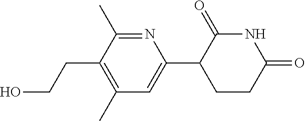 [SMILES: Cc1cc(C2CCC(=O)NC2=O)nc(C)c1CCO]

To a stirred solution 2-(2′,6′-bis(benzyloxy)-4,6-dimethyl-[2,3′-bipyridin]-5-yl)ethan-1-ol (1.4 g, 8.31 mmol) in THF (50 mL) was added Pd/C (10 wt %, 1.5 g). The resulting mixture was degassed under reduced pressure and purged with H₂ for five times, then stirred overnight at 50° C. The resulting mixture was filtered, the filter cake was washed with THF. The filtrate was concentrated under reduced pressure. And the residue was purified by column chromatography (EA/PE=30-50%) to afford 3-(5-(2-hydroxyethyl)-4,6-dimethylpyridin-2-yl)piperidine-2,6-dione (855.7 mg, 83.0%). [M+H]⁺=263.1. ¹H NMR (300 MHz, DMSO) δ 10.80 (s, 1H), 6.96 (s, 1H), 4.83-4.74 (m, 1H), 3.83 (m, 1H), 3.52 (m, 2H), 2.78 (t, J=9 Hz, 2H), 2.64-2.54 (m, 3H), 2.44 (s, 3H), 2.29 (s, 3H), 2.26-2.02 (m, 1H).

### Step 5: 2-(6-(2,6-dioxopiperidin-3-yl)-2,4-dimethylpyridin-3-yl)ethyl methanesulfonate

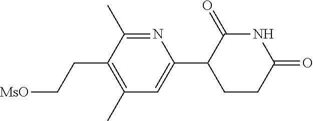 [SMILES: Cc1cc(C2CCC(=O)NC2=O)nc(C)c1CCOS(C)(=O)=O]

To a solution of 3-(5-(2-hydroxyethyl)-4,6-dimethylpyridin-2-yl)piperidine-2,6-dione (300 mg, 1.15 mmol) and Et₃N (347 mg, 3.44 mmol) in DCM (6 mL) and THF (6 mL), MsCl (172 mg, 1.49 mmol) was slowly added at 0° C. The mixture was stirred at 25° C. for 2 hours. The mixture was quenched with water (10 mL). The organic phase was separated and concentrated in vacuum. The residue was purified by prep-TLC (DCM:MeOH=20:1) to afford product (220 mg, 56.5% yield). [M+H]⁺=341.5.

### Intermediate 20: 3-(3-fluoro-5-((3R,4S)-3-fluoro-4-(piperazin-1-yl)piperidin-1-yl)-6-methylpyridin-2-yl)piperidine-2,6-dione

### Step 1: tert-butyl (3R,4S)-4-(4-benzylpiperazin-1-yl)-3-fluoropiperidine-1-carboxylate

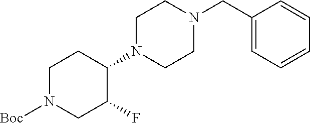 [SMILES: CC(C)(C)OC(=O)N1CC[C@H](N2CCN(Cc3ccccc3)CC2)[C@H](F)C1]

The solution of tert-butyl (3R,4S)-4-amino-3-fluoropiperidine-1-carboxylate (2.5 g, 11.46 mmol) N-benzyl-2-chloro-N-(2-chloroethyl)ethan-1-amine hydrochloride (3 g, 11.46 mmol) and NaHCO₃ (3.85 g, 45.84 mmol) in 50 mL EtOH was stirred at 80° C. for 16 hours. The mixture was concentrated, diluted with water and extracted by DCM. The organic layer was separated and concentrated. The mixture was purified by silica column chromatography (MeOH:DCM=0-4%) to afford tert-butyl (3R,4S)-4-(4-benzylpiperazin-1-yl)-3-fluoropiperidine-1-carboxylate (2 g, 5.3 mmol, 46.3%). [M+H]⁺=378.6.

### Step 2: 1-benzyl-4-((3R,4S)-3-fluoropiperidin-4-yl)piperazine

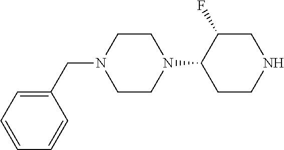 [SMILES: F[C@@H]1CNCC[C@@H]1N1CCN(Cc2ccccc2)CC1]

To a solution of tert-butyl (3R,4S)-4-(4-benzylpiperazin-1-yl)-3-fluoropiperidine-1-carboxylate (5 g, 13.2 mmol) in DCM (20 mL) was added TFA (10 mL). The reaction was stirred at room temperature for 2 hr and then concentrated in vacuo. The residue was dissolved in DCM (200 mL), washed with sat. NaHCO₃ solution (3×100 mL) and brine (50 mL), dried over Na₂SO₄, filtered and concentrated under reduced pressure to afford the product (3 g, 81.7%). [M+H]⁺=278.4.

### Step 3: 2′,6′-bis(benzyloxy)-5-((3R,4S)-4-(4-benzylpiperazin-1-yl)-3-fluoropiperidin-1-yl)-3-fluoro-6-methyl-2,3′-bipyridine

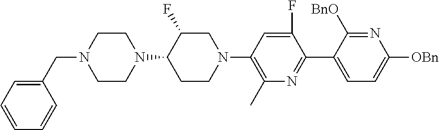 [SMILES: Cc1nc(-c2ccc(OCc3ccccc3)nc2OCc2ccccc2)c(F)cc1N1CC[C@H](N2CCN(Cc3ccccc3)CC2)[C@H](F)C1]

To a stirred solution of intermediate 11 (0.95 g, 1.8 mmol) in dioxane (20 mL) were added Cs₂CO₃ (1.2 g, 3.6 mmol), 1-benzyl-4-((3R,4S)-3-fluoropiperidin-4-yl)piperazine (0.5 g, 1.8 mmol) and Pd-Ruphos-G3 (0.3 g, 0.4 mmol) at room temperature under nitrogen atmosphere. The resulting mixture was stirred for 12 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (0-5%) to afford the product (0.95 g, 78%). [M+H]⁺=676.7.

### Step 4: tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate

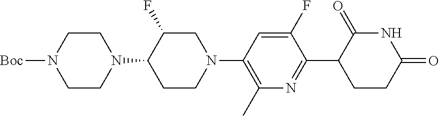 [SMILES: Cc1nc(C2CCC(=O)NC2=O)c(F)cc1N1CC[C@H](N2CCN(C(=O)OC(C)(C)C)CC2)[C@H](F)C1]

To a stirred mixture of 2′,6′-bis(benzyloxy)-5-((3R,4S)-4-(4-benzylpiperazin-1-yl)-3-fluoropiperidin-1-yl)-3-fluoro-6-methyl-2,3′-bipyridine (0.95 g, 1.4 mmol), di-tert-butyl dicarbonate (1.5 g, 7 mmol) in i-PrOH (40 mL) and DMF (40 mL) was added Pd on carbon (1 g, wet, 10 wt %). The resulting mixture was stirred under hydrogen atmosphere (1 atm) at room temperature for 24 hr. Then the mixture was filtered and the filtrate was concentrated. The residue was purified by silica gel column chromatography, eluted with MeOH/DCM (0-7%) to afford the product (0.46 g, 64.5%). [M+H]⁺=508.4.

### Step 5: 3-(3-fluoro-5-((3R,4S)-3-fluoro-4-(piperazin-1-yl)piperidin-1-yl)-6-methylpyridin-2-yl)piperidine-2,6-dione

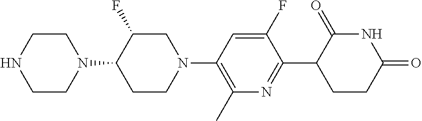 [SMILES: Cc1nc(C2CCC(=O)NC2=O)c(F)cc1N1CC[C@H](N2CCNCC2)[C@H](F)C1]

To a solution of tert-butyl 4-((3R,4S)-1-(6-(2,6-dioxopiperidin-3-yl)-5-fluoro-2-methylpyridin-3-yl)-3-fluoropiperidin-4-yl)piperazine-1-carboxylate (460 mg, 0.9 mmol) in DCM (10 mL) was added TFA (5 mL). The reaction was stirred at room temperature for 2 hr and then concentrated in vacuo. The residue was dissolved in DCM (50 mL), washed with sat. NaHCO₃ solution (3×20 mL) and brine (20 mL), dried over Na₂SO₄, filtered and concentrated under reduced pressure to afford the product (260 mg, 70.4%). [M+H]⁺=408.5.

### Intermediate 21: 3-(3-fluoro-5-((3S,4R)-3-fluoro-4-(piperazin-1-yl)piperidin-1-yl)-6-methylpyridin-2-yl)piperidine-2,6-dione

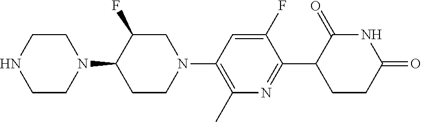 [SMILES: Cc1nc(C2CCC(=O)NC2=O)c(F)cc1N1CC[C@@H](N2CCNCC2)[C@@H](F)C1]

The title compound was prepared in a manner similar to that in Intermediate 20.

### Intermediate 22: 3-(5-((3S,4R)-3-fluoro-4-(piperazin-1-yl)piperidin-1-yl)-4,6-dimethylpyridin-2-yl)piperidine-2,6-dione

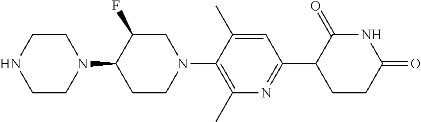 [SMILES: Cc1cc(C2CCC(=O)NC2=O)nc(C)c1N1CC[C@@H](N2CCNCC2)[C@@H](F)C1]

The title compound was prepared in a manner similar to that in Intermediate 20.

### Intermediate 23: 3-(5-((3R,4S)-3-fluoro-4-(piperazin-1-yl)piperidin-1-yl)-4-methylpyridin-2-yl)piperidine-2,6-dione

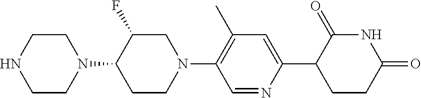 [SMILES: Cc1cc(C2CCC(=O)NC2=O)ncc1N1CC[C@H](N2CCNCC2)[C@H](F)C1]

The title compound was prepared in a manner similar to that in Intermediate 20.

### Intermediate 24: 3-(5-((3R,4S)-3-fluoro-4-(piperazin-1-yl)piperidin-1-yl)-6-methylpyridin-2-yl)piperidine-2,6-dione

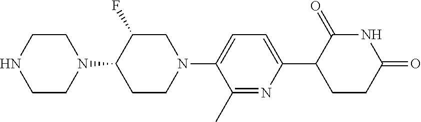 [SMILES: Cc1nc(C2CCC(=O)NC2=O)ccc1N1CC[C@H](N2CCNCC2)[C@H](F)C1]

The title compound was prepared in a manner similar to that in Intermediate 20.

### Intermediate 25: 3-(5-((R)-3,3-difluoro-4-(piperazin-1-yl)piperidin-1-yl)-6-methylpyridin-2-yl)piperidine-2,6-dione

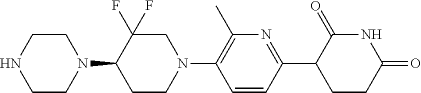 [SMILES: Cc1nc(C2CCC(=O)NC2=O)ccc1N1CC[C@@H](N2CCNCC2)C(F)(F)C1]

The title compound was prepared in a procedure similar to that in intermediate 20.

### Intermediate 26: 3-(5-((3S,4R)-3-fluoro-4-(piperazin-1-yl)piperidin-1-yl)-6-methylpyridin-2-yl)piperidine-2,6-dione

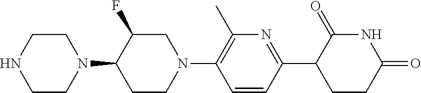 [SMILES: Cc1nc(C2CCC(=O)NC2=O)ccc1N1CC[C@@H](N2CCNCC2)[C@@H](F)C1]

The title compound was prepared in a procedure similar to that in intermediate 20.

### Intermediate 27: 3-(5-((3S,4R)-3-fluoro-4-(piperazin-1-yl)piperidin-1-yl)-4-methylpyridin-2-yl)piperidine-2,6-dione

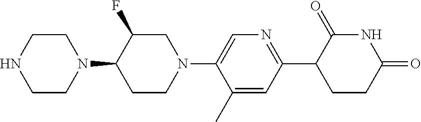 [SMILES: Cc1cc(C2CCC(=O)NC2=O)ncc1N1CC[C@@H](N2CCNCC2)[C@@H](F)C1]

The title compound was prepared in a manner similar to that in intermediate 20.

### Intermediate 28: 1-(6-(2,6-dioxopiperidin-3-yl)-2-methylpyridin-3-yl)piperidine-4-carbaldehyde

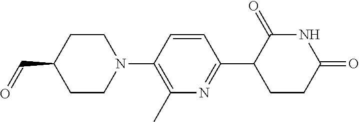

The title compound was prepared in a manner similar to that in intermediate 14.

### Intermediate 29: 1-(2-(2,6-dioxopiperidin-3-yl)-1-oxoisoindolin-5-yl)azetidine-3-carbaldehyde

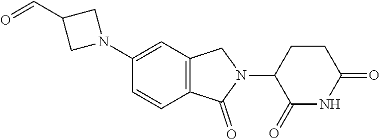 [SMILES: O=CC1CN(c2ccc3c(c2)CN(C2CCC(=O)NC2=O)C3=O)C1]

### Step 1: Methyl 2-cyano-4-(3-(hydroxymethyl) azetidin-1-yl)benzoate

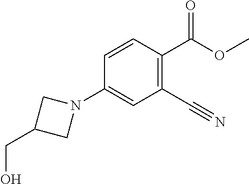 [SMILES: COC(=O)c1ccc(N2CC(CO)C2)cc1C#N]

To a stirred mixture of methyl 2-cyano-5-fluorobenzoate (20 g, 112 mmol) and azetidin-3-ylmethanol (hydrogen chloride) (14 g, 112 mmol) in DMSO (100 mL) was added DIEA (29 g, 223 mmol) in 3 h at 60° C. The resulting mixture was extracted with EtOAc (500 mL). The combined organic layers were washed with brine, dried over anhydrous Na₂SO₄. After filtration, the filtrate was concentrated under reduced pressure. Methyl 2-cyano-4-(3-(hydroxymethyl) azetidin-1-yl)benzoate (18 g, 66.7%) was obtained. [M+H]=247.0.

### Step 2: Methyl 2-formyl-4-(3-(hydroxymethyl) azetidin-1-yl)benzoate

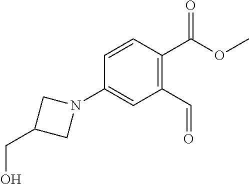 [SMILES: COC(=O)c1ccc(N2CC(CO)C2)cc1C=O]

To a stirred mixture of methyl 2-cyano-4-(3-(hydroxymethyl) azetidin-1-yl)benzoate (18 g, 72.9 mmol) in AcOH (150 mL) and H₂O (75 mL) was added Raney-Ni (15 g) in portions for 4 h at 40° C. under air atmosphere. The resulting mixture was filtered, the filter cake was washed with DCM and MeOH (300 mL). The filtrate was concentrated under reduced pressure. The filtrate was extracted with EtOAc (500 mL). The combined organic layers were washed with brine (500 mL), dried over anhydrous Na₂SO₄. After filtration, the filtrate was concentrated under reduced pressure. The residue was purified by silica gel column chromatography, eluted with CH₂Cl₂/MeOH (9:1). Methyl 2-formyl-4-(3-(hydroxymethyl) azetidin-1-yl)benzoate (10 g, 55.6%) was obtained. [M+H]⁺=250.1.

### Step 3: 3-(5-(3-(hydroxymethyl) azetidin-1-yl)-1-oxoisoindolin-2-yl) piperidine-2,6-dione

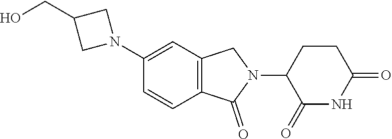 [SMILES: O=C1CCC(N2Cc3cc(N4CC(CO)C4)ccc3C2=O)C(=O)N1]

A mixture of 3-aminopiperidine-2,6-dione (hydrogen chloride) (9.9 g, 60.2 mmol) and DIEA (10.4 g, 80.3 mmol) in DMF (90 mL) was stirred for 5 h at room temperature. The mixture was acidified to pH<7 with AcOH (12 g, 201 mmol) followed by the addition of methyl 2-formyl-4-(3-(hydroxymethyl) azetidin-1-yl)benzoate (10 g, 40.2 mmol) in DMF (10 mL) at room temperature. The resulting mixture was stirred overnight at room temperature. To the above mixture was added NaBH₃CN (7.5 g, 119.35 mmol) in portions at room temperature. The resulting mixture was stirred for additional 2 h at room temperature. The reaction was quenched with water at room temperature. The resulting mixture was concentrated under reduced pressure. The residue was purified by silica gel column chromatography, eluted with CH₂Cl₂/MeOH (10:1) to afford crude product. The residue was purified by trituration with DCM. This resulted in 3-(5-(3-(hydroxymethyl) azetidin-1-yl)-1-oxoisoindolin-2-yl) piperidine-2,6-dione (4 g, 30.8%). ¹H NMR (300 MHz, DMSO) δ 10.94 (s, 1H), 7.48 (d, J=8 Hz, 1H), 6.52-6.42 (m, 2H), 5.04 (m, 1H), 4.81 (t, J=8 Hz, 1H), 4.30 (d, J=16 Hz, 1H), 4.18 (d, J=20 Hz, 1H), 3.93 (m, 2H), 3.65 (m, 2H), 3.59 (t, J=8 Hz, 2H), 2.97-2.76 (m, 2H), 2.64-2.53 (m, 1H), 2.35 (m, 1H), 1.95 (m, 1H). [M+H]⁺=330.1.

### Step 4: 1-(2-(2,6-dioxopiperidin-3-yl)-1-oxoisoindolin-5-yl)azetidine-3-carbaldehyde

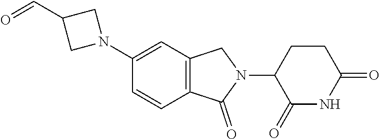 [SMILES: O=CC1CN(c2ccc3c(c2)CN(C2CCC(=O)NC2=O)C3=O)C1]

The title compound was prepared in a manner similar to that in Intermediate 33 step 2. [M+H]⁺=328.1

### Intermediate 30: (7¹R,7³S,E)-5⁶-((R)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

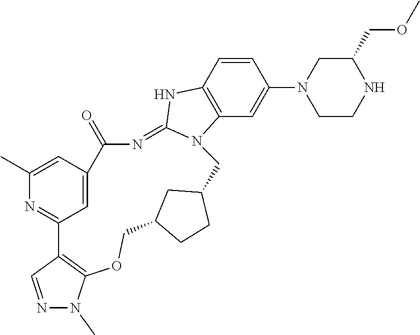

The title compound was prepared in a manner similar to that in Intermediate 2. [M+H]⁺=571.5

### Intermediate 31: 3-(4-(4-oxopiperidin-1-yl)phenoxy)piperidine-2,6-dione

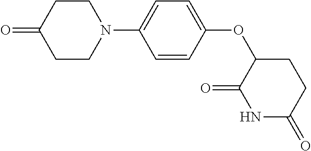 [SMILES: O=C1CCN(c2ccc(OC3CCC(=O)NC3=O)cc2)CC1]

### Step 1: 3-(4-bromophenoxy)piperidine-2,6-dione

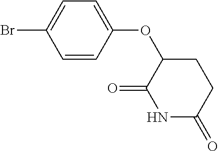 [SMILES: O=C1CCC(Oc2ccc(Br)cc2)C(=O)N1]

The solution of 4-bromophenol (2 g, 11.6 mmol), 3-bromopiperidine-2,6-dione (4.4 g, 23.1 mmol) and Cs₂CO₃ (11.3 g, 34.7 mmol) in DMF (50 mL) was stirred at 60° C. for 5 hr and then diluted with EtOAc (400 mL) and washed with brine (3×150 mL). The organic layer was dried over anhydrous Na₂SO₄, filtered and concentrated under reduced pressure. The residue was purified by silica gel column chromatography, eluted with DCM/EA (0-30%) to afford the product (1 g, 30.5%); [M+H]⁺=284.2.

### Step 2: 3-(4-(1,4-dioxa-8-azaspiro[4.5]decan-8-yl)phenoxy)piperidine-2,6-dione

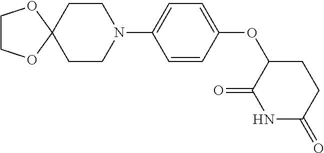 [SMILES: O=C1CCC(Oc2ccc(N3CCC4(CC3)OCCO4)cc2)C(=O)N1]

To a stirred solution of 3-(4-bromophenoxy)piperidine-2,6-dione (900 mg, 3.2 mmol) and 1,4-dioxa-8-azaspiro[4.5]decane (544 mg, 1.3 mmol) in DMA (20 mL) were added Cs₂CO₃ (2 g, 6.3 mmol), Ruphos (591 mg, 0.24 mmol) and Pd₂(dba)₃ (580 mg, 0.6 mmol) at room temperature under nitrogen atmosphere. The resulting mixture was stirred for 6 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (25:1) to afford the product (130 mg, 11.9%); [M+H]+=347.3.

### Step 3: 3-(4-(4-oxopiperidin-1-yl)phenoxy)piperidine-2,6-dione

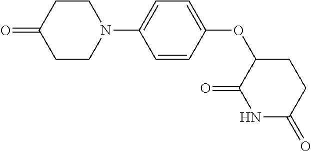 [SMILES: O=C1CCN(c2ccc(OC3CCC(=O)NC3=O)cc2)CC1]

The solution of 3-(4-(1,4-dioxa-8-azaspiro[4.5]decan-8-yl)phenoxy)piperidine-2,6-dione (130 mg, 0.4 mmol) in HCl aq. (8 N, 5 mL) was stirred at r.t. for 2 hr and DCM (100 mL) was added. The resulting mixture was adjusted to pH=7 with NaHCO₃ aq. and the organic layer was separated and dried over anhydrous Na₂SO₄, filtered and concentrated under reduced pressure to afford the crude product (100 mg, 88.1%); [M+H]⁺=303.2.

### Intermediate 32: 3-((3-fluoro-4-(4-oxopiperidin-1-yl)phenyl)amino)piperidine-2,6-dione

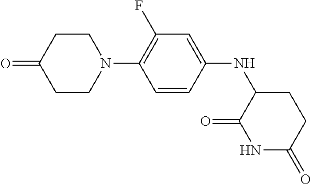 [SMILES: O=C1CCN(c2ccc(NC3CCC(=O)NC3=O)cc2F)CC1]

### Step 1: 8-(2-fluoro-4-nitrophenyl)-1,4-dioxa-8-azaspiro[4.5]decane

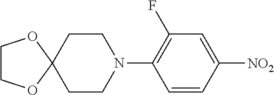 [SMILES: O=[N+]([O-])c1ccc(N2CCC3(CC2)OCCO3)c(F)c1]

To the solution of 1,2-difluoro-4-nitrobenzene (3.0 g, 18.9 mmol) and 1,4-dioxa-8-azaspiro[4.5]decane (4.0 g, 28.3 mmol) in 60 mL DMF were added K₂CO₃ (5.2 g, 37.8 mmol). The resulting mixture was stirred at 80° C. for 16 hours. The reaction was poured into water (150 mL), and the precipitate was filtered, washed with water and dried under air to afford the crude product (5.3 g, 96.2%). [M+H]⁺=283.5.

### Step 2: 3-fluoro-4-(1,4-dioxa-8-azaspiro[4.5]decan-8-yl)aniline

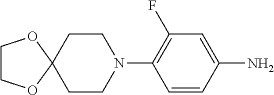 [SMILES: Nc1ccc(N2CCC3(CC2)OCCO3)c(F)c1]

To the solution of 8-(2-fluoro-4-nitrophenyl)-1,4-dioxa-8-azaspiro[4.5]decane (5.1 g, 18.0 mmol) in 40 mL DCM and 40 mL MeOH was added Pd/C (2.5 g, 10 wt. %, wet). The mixture was stirred at RT for 12 hours under hydrogen atmosphere (balloon). The mixture was filtered through a celite pad and washed with DCM. The filtrate was concentrated in vacuum to afford the crude product (4.4 g, 96.5%). [M+H]⁺=253.5.

### Step 3: 3-((3-fluoro-4-(1,4-dioxa-8-azaspiro[4.5]decan-8-yl)phenyl)amino)piperidine-2,6-dione

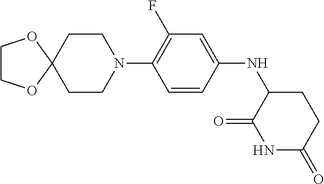 [SMILES: O=C1CCC(Nc2ccc(N3CCC4(CC3)OCCO4)c(F)c2)C(=O)N1]

To the solution of 3-fluoro-4-(1,4-dioxa-8-azaspiro[4.5]decan-8-yl)aniline (700 mg, 2.8 mmol) and 3-bromopiperidine-2,6-dione (525 mg, 2.8 mmol) in 10 mL DMF were added Na₂CO₃ (595 mg, 5.6 mmol). The resulting mixture was heated at 70° C. for 16 hours. The mixture was quenched with water and extracted with EA (2×50 mL). The combined organic phase was washed with brine (2×30 mL), concentrated in vacuum and purified with combiflash (DCM:EA=1:2) to afford the title compound (345 mg, 34.5%). [M+H]⁺=364.5.

### Step 4: 3-((3-fluoro-4-(4-oxopiperidin-1-yl)phenyl)amino)piperidine-2,6-dione

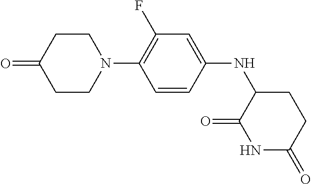 [SMILES: O=C1CCN(c2ccc(NC3CCC(=O)NC3=O)cc2F)CC1]

3-((3-fluoro-4-(1,4-dioxa-8-azaspiro[4.5]decan-8-yl)phenyl)amino)piperidine-2,6-dione (345 mg, 0.95 mmol) was placed in 100 mL round bottom flask with a magnetic stir bar. Then 10 mL 8 N HCl aqueous was added. The mixture was stirred at room temperature for 2 hours. The mixture was added dropwise to sat. aq. NaHCO₃ solution and finally pH=6-7. The liquid was extracted with DCM (2×50 mL). The combined organic phase was concentrated in vacuum and purified with combiflash (DCM:MeOH=25:1) to afford the title compound (300 mg, 99.0%). [M+H]⁺=320.5.

### Intermediate 33: 1-(2-(2,6-dioxopiperidin-3-yl)-1,3-dioxoisoindolin-5-yl)azetidine-3-carbaldehyde

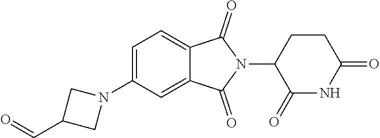 [SMILES: O=CC1CN(c2ccc3c(c2)C(=O)N(C2CCC(=O)NC2=O)C3=O)C1]

### Step 1: 2-(2,6-dioxopiperidin-3-yl)-5-(3-(hydroxymethyl)azetidin-1-yl)isoindoline-1,3-dione

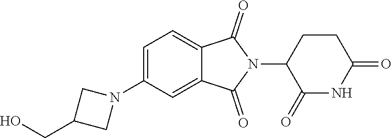 [SMILES: O=C1CCC(N2C(=O)c3ccc(N4CC(CO)C4)cc3C2=O)C(=O)N1]

A mixture of 2-(2,6-dioxopiperidin-3-yl)-5-fluoroisoindoline-1,3-dione (2.76 g, 1 mmol), azetidin-3-ylmethanol (870 mg, 1 mmol) and DIEA (2.85 g, 2 mmol) in DMSO (30 mL) was stirred in a round bottom flask at 100° C. for 12 hours under N₂. The mixture was diluted with H₂O and extracted with EtOAc (100 mL×3). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (10:1). 2-(2,6-dioxopiperidin-3-yl)-5-(3-(hydroxymethyl)azetidin-1-yl)isoindoline-1,3-dione (3 g, 87%) was obtained. [M+H]⁺=344.3

### Step 2: 1-(2-(2,6-dioxopiperidin-3-yl)-1,3-dioxoisoindolin-5-yl)azetidine-3-carbaldehyde

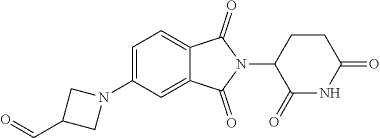 [SMILES: O=CC1CN(c2ccc3c(c2)C(=O)N(C2CCC(=O)NC2=O)C3=O)C1]

A mixture of 2-(2,6-dioxopiperidin-3-yl)-5-(3-(hydroxymethyl)azetidin-1-yl)isoindoline-1,3-dione (1.71 g, 0.5 mmol), 2-Iodoxybenzoic acid (2.8 g, 1 mmol) in DMSO (15 mL) was stirred in a round bottom flask at 25° C. for 12 hours under N₂. The mixture was diluted with H₂O and extracted with DCM (100 mL×3). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH (10:1) to afford the title compound (1.5 g, 88%). [M+H]⁺=342.3

### Intermediate 34: (7¹R,7³S,E)-1¹,1³,2⁶-trimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

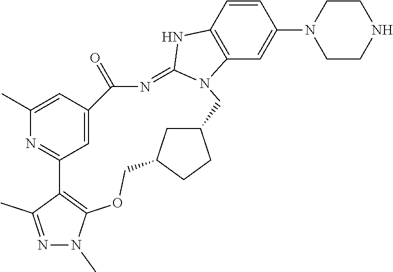 [SMILES: Cc1cc2cc(n1)-c1c(C)nn(C)c1OC[C@H]1CC[C@H](C1)CN1/C(=N/C2=O)Nc2ccc(N3CCNCC3)cc21]

The title compound was prepared in a manner similar to that in Intermediate 40 with intermediate 39. [M+H]⁺=541.5.

### Intermediate 35: (7¹R,7³S,E)-5⁶-((R)-3-(hydroxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

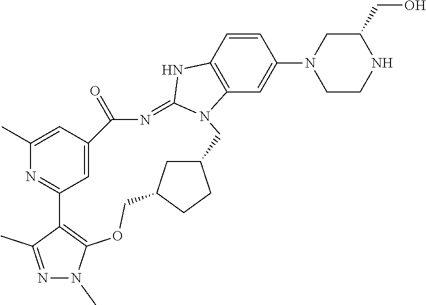 [SMILES: Cc1cc2cc(n1)-c1c(C)nn(C)c1OC[C@H]1CC[C@H](C1)CN1/C(=N/C2=O)Nc2ccc(N3CCN[C@@H](CO)C3)cc21]

The title compound was prepared in a manner similar to that in Intermediate 2. [M+H]⁺=557.5.

### Intermediate 36: 3-(5-((3S,4S)-3-fluoro-4-(piperazin-1-yl)piperidin-1-yl)-6-methylpyridin-2-yl)piperidine-2,6-dione

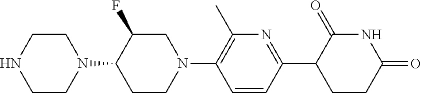 [SMILES: Cc1nc(C2CCC(=O)NC2=O)ccc1N1CC[C@H](N2CCNCC2)[C@@H](F)C1]

The title compound was prepared in a manner similar to that in intermediate 20. [M+H]⁺=390.3.

### Intermediate 37: (7¹R,7³S,E)-5⁶-((S)-3-(methoxymethyl)piperazin-1-yl)-1¹,2⁶-dimethyl-1³-(trifluoromethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

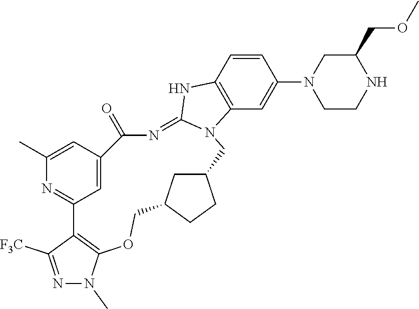 [SMILES: COC[C@@H]1CN(c2ccc3c(c2)N2C[C@@H]4CC[C@H](COc5c(c(C(F)(F)F)nn5C)-c5cc(cc(C)n5)C(=O)/N=C/2N3)C4)CCN1]

The title compound was prepared in a manner similar to that in Intermediates 1 and 2. [M+H]⁺=639.5.

### Intermediate 38: 2-(6-(2,6-dioxopiperidin-3-yl)-5-fluoro-2-methylpyridin-3-yl)acetaldehyde

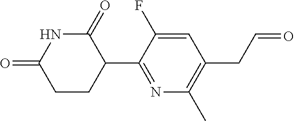 [SMILES: Cc1nc(C2CCC(=O)NC2=O)c(F)cc1CC=O]

### Step 1: 2′,6′-bis(benzyloxy)-5-(2,2-dimethoxyethyl)-3-fluoro-6-methyl-2,3′-bipyridine

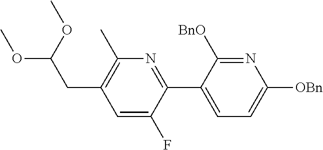 [SMILES: COC(Cc1cc(F)c(-c2ccc(OCc3ccccc3)nc2OCc2ccccc2)nc1C)OC]

To a mixture of 2′,6′-bis(benzyloxy)-5-bromo-3-fluoro-6-methyl-2,3′-bipyridine (700 mg, 1.5 mmol, obtained from the same way of WO2023098656 A1), 3-bromo-1,1-dimethoxypropane (400 mg, 2.2 mmol), NiI₂ (91 mg, 0.3 mmol), HCl salt of picolinimidamide (46 mg, 0.3 mmol), NaI (87 mg, 0.58 mmol), Mn (160 mg, 3 mmol) in DMA (15 mL) was added TFA (25 mg, 0.25 mmol) under N₂. The resulting mixture was heated to 100° C. for 3 hrs under N₂. After cooling to r.t, the reaction was diluted with EA (60 mL) and then washed with brine (20 mL×3), dried over Na₂SO₄, filtered and concentrated in vacuum. The residue was purified by silica gel column (PE:EA=5:1) to afford the product (560 mg, 78.5%); [M+H]⁺=489.6.

### Step 2: 3-(5-(2,2-dimethoxyethyl)-3-fluoro-6-methylpyridin-2-yl)piperidine-2,6-dione

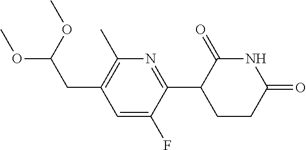 [SMILES: COC(Cc1cc(F)c(C2CCC(=O)NC2=O)nc1C)OC]

Under N₂, to a solution of 2′,6′-bis(benzyloxy)-5-(2,2-dimethoxyethyl)-3-fluoro-6-methyl-2,3′-bipyridine (560 mg, 1.15 mmol) in DMF (10 mL)/i-PrOH (10 mL) was added 10% Pd/C (500 mg) at 25° C. Then the mixture was exchanged with H₂ twice and stirred under H₂ atmosphere at 50° C. for 12 hours. The mixture was filtered through a pad of Celite and washed with MeOH (50 mL). The filtrate was concentrated under vacuum to afford the crude product (120 mg, 33.7%). [M+H]⁺=311.4.

### Step 3: 2-(6-(2,6-dioxopiperidin-3-yl)-5-fluoro-2-methylpyridin-3-yl)acetaldehyde

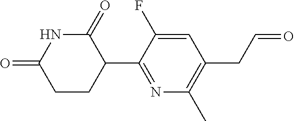 [SMILES: Cc1nc(C2CCC(=O)NC2=O)c(F)cc1CC=O]

The solution of 3-(5-(2,2-dimethoxyethyl)-3-fluoro-6-methylpyridin-2-yl)piperidine-2,6-dione (120 mg, 0.4 mmol) in HCl aq. (8 N, 5 mL) was stirred at r.t. for 2 hr and DCM (100 mL) was added. The resulting mixture was adjusted to pH=7 with NaHCO₃ aq. and the organic layer was separated and dried over anhydrous Na₂SO₄, filtered and concentrated under reduced pressure to afford the product (100 mg, 97.8%); [M+H]⁺=265.2.

### Intermediate 39: (7¹R,7³S,E)-5⁶-bromo-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

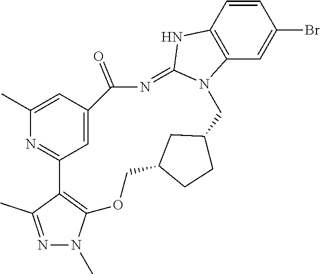 [SMILES: Cc1cc2cc(n1)-c1c(C)nn(C)c1OC[C@H]1CC[C@H](C1)CN1/C(=N/C2=O)Nc2ccc(Br)cc21]

### Step 1: methyl 2-(5-hydroxy-1,3-dimethyl-1H-pyrazol-4-yl)-6-methylisonicotinate

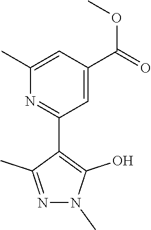 [SMILES: COC(=O)c1cc(C)nc(-c2c(C)nn(C)c2O)c1]

To the solution of methyl 2-chloro-6-methylisonicotinate (1.85 g, 10 mmol), 1,3-dimethyl-1H-pyrazol-5-ol (2.24 g, 20 mmol) and Na₂CO₃ (2.12 g, 20 mmol) in 50 mL anisole, Pd(dppf)Cl₂ (1.46 g, 2 mmol) was added at N₂ atmosphere. The mixture was stirred at 130° C. for 16 hours at N₂ atmosphere. The mixture was filtered and the filtrate was concentrated in vacuum. The residue was purified by silica column chromatography (MeOH:DCM=0-7%) to afford methyl 2-(5-hydroxy-1,3-dimethyl-1H-pyrazol-4-yl)-6-methylisonicotinate (990 mg, 3.79 mmol, 37.9% yield). [M+H]⁺=262.2.

### Step 2: methyl 2-(5-(((1S,3R)-3-(((5-bromo-2-nitrophenyl)amino)methyl)cyclopentyl)methoxy)-1,3-dimethyl-1H-pyrazol-4-yl)-6-methylisonicotinate

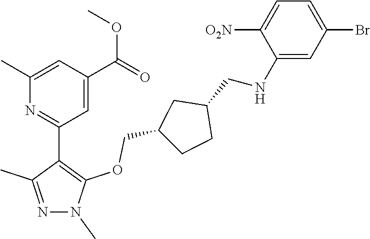 [SMILES: COC(=O)c1cc(C)nc(-c2c(C)nn(C)c2OC[C@H]2CC[C@@H](CNc3cc(Br)ccc3[N+](=O)[O-])C2)c1]

To the solution of methyl 2-(5-hydroxy-1,3-dimethyl-1H-pyrazol-4-yl)-6-methylisonicotinate (990 mg, 3.79 mmol), ((1S,3R)-3-(((5-bromo-2-nitrophenyl)amino)methyl)cyclopentyl)methanol (1.24 g, 3.79 mmol) and PPh₃ (1.19 g, 4.55 mmol) in 30 mL THF, DIAD (920 mg, 4.55 mmol) was added at 0° C. The mixture was stirred at room temperature for 4 hours. The mixture was concentrated in vacuum and purified by silica column chromatography (MeOH:DCM=0-5%) to afford methyl 2-(5-(((1S,3R)-3-(((5-bromo-2-nitrophenyl)amino)methyl)cyclopentyl)methoxy)-1,3-dimethyl-1H-pyrazol-4-yl)-6-methylisonicotinate (2.6 g crude with PPh₃O). [M+H]⁺=572.2.

### Step 3: methyl 2-(5-(((1S,3R)-3-(((2-amino-5-bromophenyl)amino)methyl)cyclopentyl)methoxy)-1,3-dimethyl-1H-pyrazol-4-yl)-6-methylisonicotinate

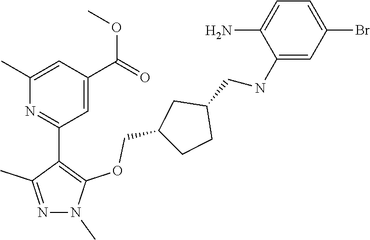 [SMILES: COC(=O)c1cc(C)nc(-c2c(C)nn(C)c2OC[C@H]2CC[C@@H](CNc3cc(Br)ccc3N)C2)c1]

To the solution of methyl 2-(5-(((1S,3R)-3-(((5-bromo-2-nitrophenyl)amino)methyl)cyclopentyl)methoxy)-1,3-dimethyl-1H-pyrazol-4-yl)-6-methylisonicotinate (2.6 g crude with PPh₃O) in 50 mL THF, Raney Ni was added. The mixture was stirred at room temperature for 1 hour at H₂ atmosphere. The mixture was filtered by celite and the filtrate was concentrated in vacuum to afford methyl 2-(5-(((1S,3R)-3-(((2-amino-5-bromophenyl)amino)methyl)cyclopentyl)methoxy)-1,3-dimethyl-1H-pyrazol-4-yl)-6-methylisonicotinate (2.5 g crude). [M+H]⁺=542.2.

### Step 4: methyl 2-(5-(((1S,3R)-3-((6-bromo-2-imino-2,3-dihydro-1H-benzo[d]imidazol-1-yl)methyl)cyclopentyl)methoxy)-1,3-dimethyl-1H-pyrazol-4-yl)-6-methylisonicotinate

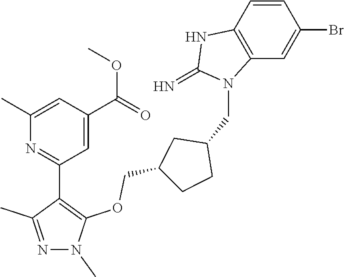 [SMILES: COC(=O)c1cc(C)nc(-c2c(C)nn(C)c2OC[C@H]2CC[C@@H](Cn3c(=N)[nH]c4ccc(Br)cc43)C2)c1]

To the solution of methyl 2-(5-(((1S,3R)-3-(((2-amino-5-bromophenyl)amino)methyl)cyclopentyl)methoxy)-1,3-dimethyl-1H-pyrazol-4-yl)-6-methylisonicotinate (2.5 g crude) in 30 mL MeOH, BrCN (630 mg, 6 mmol) was added. The mixture was stirred at room temperature for 4 hours. The mixture was concentrated in vacuum. The residue was dissolved in DCM (100 mL) and washed with sat. aq. K₂CO₃ (50 mL×3) and separated. The organic layer was dried and concentrated and purified by silica column chromatography (MeOH:DCM=0-8%) to afford methyl 2-(5-(((1S,3R)-3-((6-bromo-2-imino-2,3-dihydro-1H-benzo[d]imidazol-1-yl)methyl)cyclopentyl)methoxy)-1,3-dimethyl-1H-pyrazol-4-yl)-6-methylisonicotinate (1.6 g, 2.83 mmol). [M+H]⁺=567.2.

### Step 5: (7¹R,7³S,E)-5⁶-bromo-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

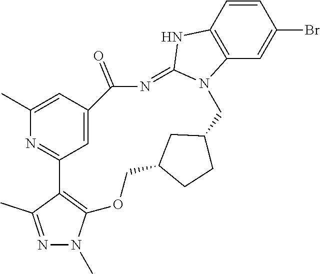 [SMILES: Cc1cc2cc(n1)-c1c(C)nn(C)c1OC[C@H]1CC[C@H](C1)CN1/C(=N/C2=O)Nc2ccc(Br)cc21]

To the solution of methyl 2-(5-(((1S,3R)-3-((6-bromo-2-imino-2,3-dihydro-1H-benzo[d]imidazol-1-yl)methyl)cyclopentyl)methoxy)-1,3-dimethyl-1H-pyrazol-4-yl)-6-methylisonicotinate (1.6 g, 2.83 mmol) in 30 mL THF, 5 mL 1N LiHMDS in THF was added. The mixture was stirred at room temperature for 1 hour. The mixture was quenched with sat. aq. NH₄Cl (30 mL) and concentrated. The residue was dissolved in DCM (70 mL) and washed with brine (40 mL×3). The organic layer was separated and concentrated and purified by silica column chromatography (MeOH:DCM=0-4%) to afford (7¹R,7³S,E)-5⁶-bromo-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one (780 mg, 1.46 mmol, 51.6% yield). [M+H]⁺=535.2.

### Intermediate 40: (7¹R,7³S,E)-5⁶-((S)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

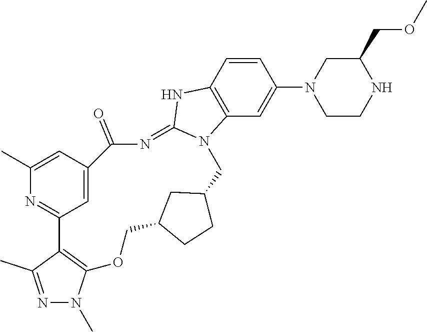

The title compound was prepared in a manner similar to that in Intermediate 2 with intermediate 39. [M+H]+=585.1.

### Intermediate 41: 3-(5-((S)-3,3-difluoro-4-(piperazin-1-yl)piperidin-1-yl)-6-methylpyridin-2-yl)piperidine-2,6-dione

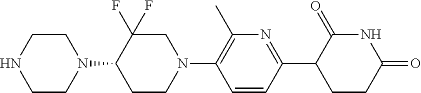 [SMILES: Cc1nc(C2CCC(=O)NC2=O)ccc1N1CC[C@H](N2CCNCC2)C(F)(F)C1]

The title compound was prepared in a procedure similar to that in intermediate 20. [M+H]⁺=408.1.

### Intermediate 42: 3-(2,6-difluoro-4-((3R,4S)-3-fluoro-4-(piperazin-1-yl)piperidin-1-yl)phenyl)piperidine-2,6-dione

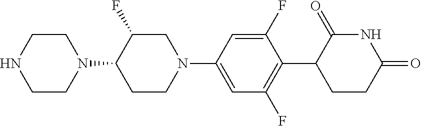 [SMILES: O=C1CCC(c2c(F)cc(N3CC[C@H](N4CCNCC4)[C@H](F)C3)cc2F)C(=O)N1]

### Step 1: 1-benzyl-4-((3R,4S)-1-(4-(2,6-bis(benzyloxy)pyridin-3-yl)-3,5-difluorophenyl)-3-fluoropiperidin-4-yl)piperazine

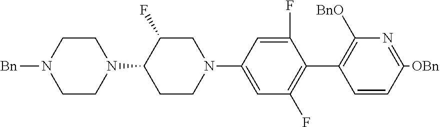 [SMILES: Fc1cc(N2CC[C@H](N3CCN(Cc4ccccc4)CC3)[C@H](F)C2)cc(F)c1-c1ccc(OCc2ccccc2)nc1OCc1ccccc1]

To the solution of 2,6-bis(benzyloxy)-3-(4-bromo-2,6-difluorophenyl)pyridine (1.0 g, 2.1 mmol), 1-benzyl-4-((3R,4S)-3-fluoropiperidin-4-yl)piperazine (694 mg, 2.5 mmol) and Cs₂CO₃ (2.0 g, 6.3 mmol) in 20 mL 1,4-dioxane, were added Pd-G3 RuPhos (351 mg, 0.42 mmol) and RuPhos (392 mg, 0.84 mmol). The mixture was stirred at 100° C. for 16 hours under N₂. The mixture was filtered through a celite pad and washed with DCM. The filtrate was concentrated under reduced pressure to give the crude residue, which was purified by silica column chromatography (DCM:MeOH=100:1-20:1) to afford the product (1.1 g, 78.0%). [M+H]⁺=679.5.

### Step 2: tert-butyl 4-((3R,4S)-1-(4-(2,6-dioxopiperidin-3-yl)-3,5-difluorophenyl)-3-fluoropiperidin-4-yl)piperazine-1-carboxylate

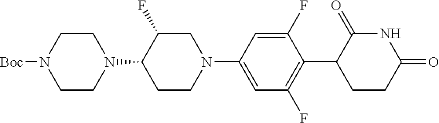 [SMILES: CC(C)(C)OC(=O)N1CCN([C@H]2CCN(c3cc(F)c(C4CCC(=O)NC4=O)c(F)c3)C[C@H]2F)CC1]

To the solution of 1-benzyl-4-((3R,4S)-1-(4-(2,6-bis(benzyloxy)pyridin-3-yl)-3,5-difluorophenyl)-3-fluoropiperidin-4-yl)piperazine (1.1 g, 1.6 mmol) and (Boc)₂O (1.0 g, 4.8 mmol) in 20 mL DMF and 20 mL iPrOH was added Pd/C (0.8 g, 10 wt. %, wet). The mixture was stirred at 50° C. for 16 hours under hydrogen atmosphere (balloon). The mixture was cooled to room temperature and filtered by celite. The filtrate was concentrated in vacuum to give the crude residue, which was purified by silica gel column chromatography (DCM:MeOH=100:0-20:1) to afford the product (790 mg, 95.5%). [M+H]⁺=511.5.

### Step 3: 3-(2,6-difluoro-4-((3R,4S)-3-fluoro-4-(piperazin-1-yl)piperidin-1-yl)phenyl)piperidine-2,6-dione

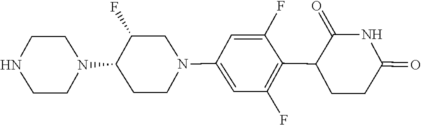 [SMILES: O=C1CCC(c2c(F)cc(N3CC[C@H](N4CCNCC4)[C@H](F)C3)cc2F)C(=O)N1]

To a solution of 4-((3R,4S)-1-(4-(2,6-dioxopiperidin-3-yl)-3,5-difluorophenyl)-3-fluoropiperidin-4-yl)piperazine-1-carboxylate (790 mg, 1.5 mmol) in DCM (6 mL) was added TFA (2 mL). The mixture was stirred at room temperature for 1 h. The mixture was concentrated and basified with sat. aq. NaHCO₃. The liquid was extracted with DCM/CF₃CH₂OH (3×30 mL). The combined organic phase was dried over Na₂SO₄, filtered and concentrated in vacuum to afford the title product (600 mg, 94.5%). [M+H]⁺=411.5.

### Intermediate 43: (7¹R,7³S,E)-1³-(difluoromethyl)-5⁶-((S)-3-(methoxymethyl)piperazin-1-yl)-1¹,2⁶-dimethyl-5²,5³-dihydro-1¹H,5¹H-9-oxa-4-aza-5(2,1)-benzol[d]imidazola-2(2,4)-pyridina-1(4,5)-pyrazola-7(1,3)-cyclopentanacyclononaphan-3-one

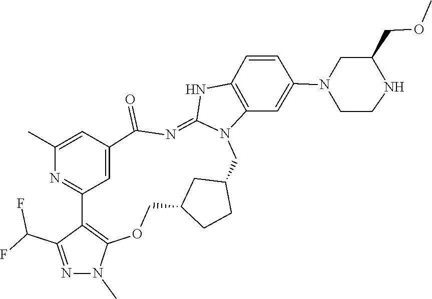 [SMILES: COC[C@@H]1CN(c2ccc3c(c2)N2C[C@@H]4CC[C@H](COc5c(c(C(F)F)nn5C)-c5cc(cc(C)n5)C(=O)/N=C/2N3)C4)CCN1]

The title compound was prepared in a manner similar to that in Intermediate 40. [M+H]⁺=621.3.

### Intermediate 44: (R)-1-(4-(2,6-dioxopiperidin-3-yl)-3,5-difluorophenyl)azetidine-3-carbaldehyde

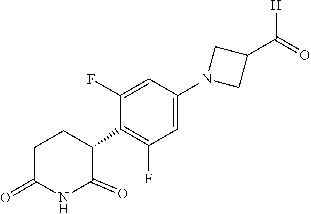 [SMILES: O=CC1CN(c2cc(F)c([C@H]3CCC(=O)NC3=O)c(F)c2)C1]

### Step 1: methyl 1-(4-(2,6-bis(benzyloxy)pyridin-3-yl)-3,5-difluorophenyl)azetidine-3-carboxylate

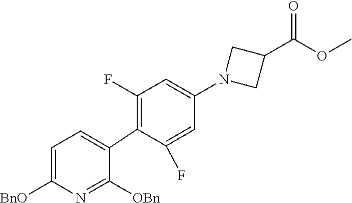 [SMILES: COC(=O)C1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (3.00 g, 6.22 mmol), methyl azetidine-3-carboxylate hydrochloride (1.41 g, 9.33 mmol), Cs₂CO₃ (6.06 g, 18.7 mmol) and RuPhos Pd G3 (520.7 mg, 0.62 mmol) in toluene (50 mL) was stirred overnight at 100° C. under nitrogen atmosphere. The resulting mixture was diluted with brine (300 mL) and extracted with EtOAc (100 mL×3). The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+H]⁺=517.1.

### Step 2: 1-(4-(2,6-bis(benzyloxy)pyridin-3-yl)-3,5-difluorophenyl)azetidine-3-carboxylic acid

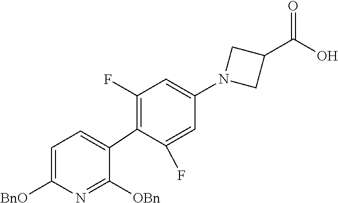 [SMILES: O=C(O)C1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiOH H₂O (168 mg, 4 mmol) in 10 mL water dropwise at room temperature. Then the mixture was stirred for 2 hours. The resulting mixture was concentrated in vacuum. The water layer was adjusted to pH<5 with 1N HCl and then extracted with EtOAc (3×40 mL). The combined organic layers were washed with brine, dried over anhydrous Na₂SO₄. After filtration, the filtrate was concentrated under reduced pressure to afford the product (1.4 g, 85%), which was used in next step without further purification. [M+H]⁺=503.2.

### Step 3: (R)-1-(4-(2,6-dioxopiperidin-3-yl)-3,5-difluorophenyl)azetidine-3-carboxylic acid

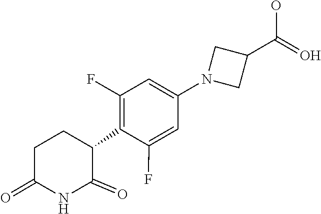 [SMILES: O=C1CC[C@H](c2c(F)cc(N3CC(C(=O)O)C3)cc2F)C(=O)N1]

To a solution of 1-(4-(2,6-bis(benzyloxy)pyridin-3-yl)-3,5-difluorophenyl)azetidine-3-carboxylic acid (1.40 g, 2.79 mmol) in iPrOH (20 mL) and DCM (20 mL) was added Pd/C (1.0 g, 10% wt), and the mixture was stirred at room temperature under hydrogen atmosphere for 48 hours. The resulting mixture was filtered, the filter cake was washed with MeOH (20 mL). The filtrate was concentrated under reduced pressure and purified by chiral HPLC (analytical method: CHIRALPAK AD-3 3.0*100 mm, 3 m, MeOH (0.1% DEA)), and the title compound corresponded to peak A @ 1.849 min/254 nm (190 mg, 22%). [M+H]⁺=325.3.

### Step 4: (R)-3-(2,6-difluoro-4-(3-(hydroxymethyl)azetidin-1-yl)phenyl)piperidine-2,6-dione

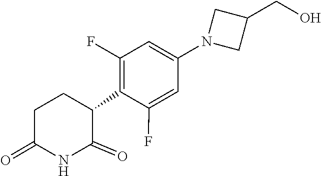 [SMILES: O=C1CC[C@H](c2c(F)cc(N3CC(CO)C3)cc2F)C(=O)N1]

To the solution of (R)-1-(4-(2,6-dioxopiperidin-3-yl)-3,5-difluorophenyl)azetidine-3-carboxylic acid (6.5 g, 20 mmol) in THF was added BH₃.THF (30 mL, 1 M in THF) dropwise at 0° C. The reaction mixture was stirred at room temperature overnight. Then the mixture was quenched with MeOH (20 mL) and diluted with DCM (150 mL), washed with sat. aq. NaHCO₃ (3×100 mL) and brine (100 mL). The organic layer was dried over anhydrous Na₂SO₄, filtered and concentrated under reduced pressure to give the crude product, which was purified by silica gel column chromatography, eluted with DCM/MeOH (30:1 to 15:1) to afford the title product (3.8 g, 61.2%). [M+H]⁺=311.2.

### Step 5: (R)-1-(4-(2,6-dioxopiperidin-3-yl)-3,5-difluorophenyl)azetidine-3-carbaldehyde

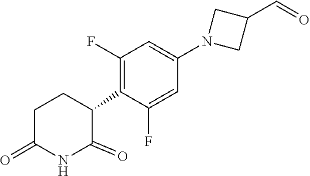 [SMILES: O=CC1CN(c2cc(F)c([C@H]3CCC(=O)NC3=O)c(F)c2)C1]

A mixture of (R)-3-(2,6-difluoro-4-(3-(hydroxymethyl)azetidin-1-yl)phenyl)piperidine-2,6-dione (3.8 g, 12.3 mmol) and IBX (6.8 g, 24.6 mmol) in DMSO (80 mL) was stirred in a flask at room temperature overnight. The reaction was quenched with water and the mixture was extracted with DCM (60 mL×3). The combined organic layers were washed with sat. aq. Na₂S₂O₃ (100 mL), sat. aq. NaHCO₃ (100 mL×2), sat. aq. NaCl (100 mL×2), dried over anhydrous Na₂SO₄, filtered and concentrated under vacuum to afford the product (2.3 g, 70.1%), which was used without further purification. [M+H]⁺=309.1.

### Intermediate 45: (7¹R,7³S,E)-5⁵-fluoro-5⁶-((S)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

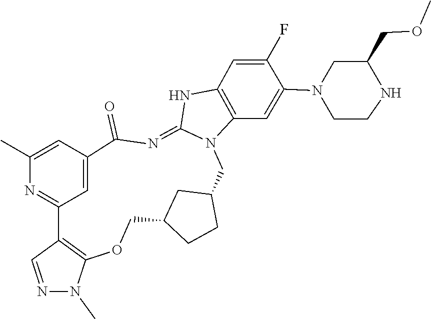

The title compound was prepared in a manner similar to that in Intermediates 1 and 2. [M+H]⁺=589.3.

### Intermediate 46: 3-(3-fluoro-4-(4-oxopiperidin-1-yl)phenoxy)piperidine-2,6-dione

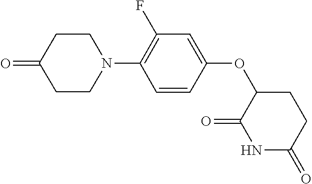 [SMILES: O=C1CCN(c2ccc(OC3CCC(=O)NC3=O)cc2F)CC1]

The title compound was prepared in a manner similar to that in Intermediate 31. [M+H]⁺=321.2

### Intermediate 47: (7¹R,7³S,E)-5⁶-((R)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

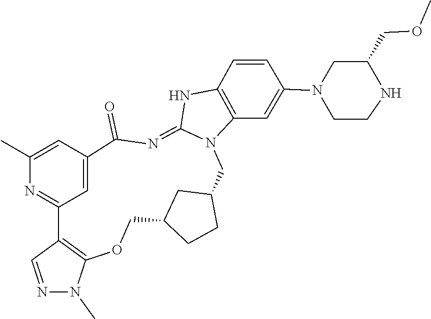

### Step 1: tert-butyl (R)-2-(methoxymethyl)-4-((7¹R,7³S,E)-1¹,1³2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate

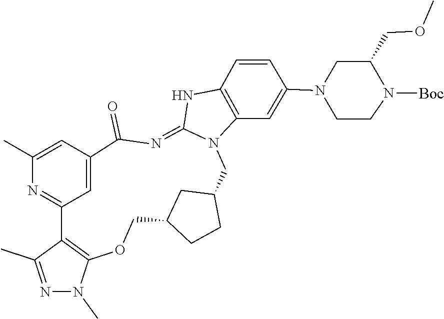

To the solution of intermediate 39 (150 mg, 0.28 mmol), tert-butyl (R)-2-(methoxymethyl)piperazine-1-carboxylate (129 mg, 0.56 mmol) and tBuONa (135 mg, 1.4 mmol) in 5 mL DMA was added Pd₂(dba)₃ (51 mg, 0.056 mmol) and RuPhos (52 mg, 0.12 mmol). The mixture was stirred at 90° C. for 1 hour. The mixture was concentrated in vacuum and purified by silica column chromatography (MeOH:DCM=0-4%) to afford the title compound (180 mg, 93.8%). [M+H]⁺=685.2.

### Step 2: (7¹R,7³S,E)-5⁶-((R)-3-(methoxymethyl)piperazin-1-yl)-1¹,1³2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

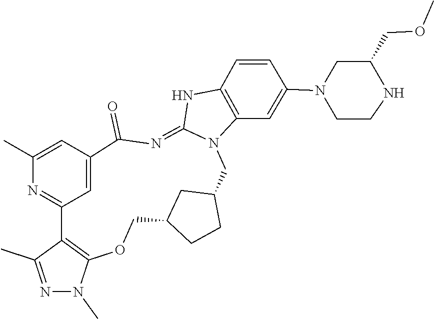

To the solution of tert-butyl (R)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate (180 mg, 0.26 mmol) in 2 mL DCM was added 2 mL TFA. The mixture was stirred at room temperature for 15 minutes. The mixture was concentrated and dissolved in DCM (20 mL). The residue was washed with sat. aq. NaHCO₃ (10 mL×3) and separated. The organic layer was concentrated and purified by silica column chromatography ((MeOH+2% NH₃·H₂O):DCM=0-15%) to afford the title compound (130 mg, 84.6%). [M+H]⁺=585.2.

### Intermediate 48: 3-((3,5-difluoro-4-(4-oxopiperidin-1-yl)phenyl)amino)piperidine-2,6-dione

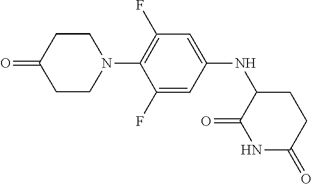 [SMILES: O=C1CCN(c2c(F)cc(NC3CCC(=O)NC3=O)cc2F)CC1]

The title compound was prepared in a manner similar to that in Intermediate 32. [M+H]⁺=338.2.

### Intermediate 49: (7¹R,7³S,E)-5⁶-((R)-2-(hydroxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

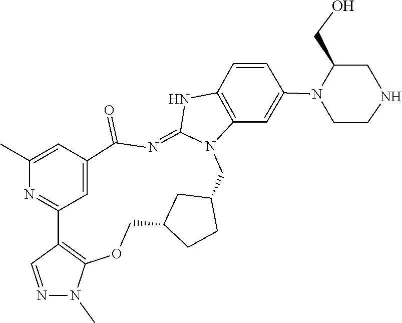 [SMILES: Cc1cc2cc(n1)-c1cnn(C)c1OC[C@H]1CC[C@H](C1)CN1/C(=N/C2=O)Nc2ccc(N3CCNC[C@@H]3CO)cc21]

The title compound was prepared in a manner similar to that in Intermediate 2. [M+H]⁺=557.2.

### Intermediate 50: (7¹R,7³S,E)-5⁶-((R)-3-(methoxymethyl)piperazin-1-yl)-1¹,2⁶-dimethyl-1³-(trifluoromethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

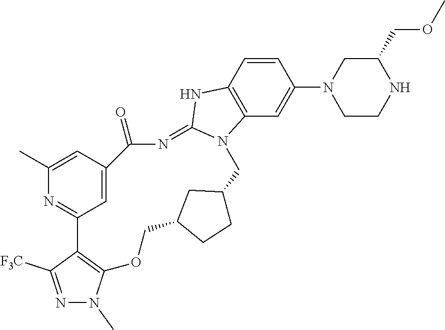 [SMILES: COC[C@H]1CN(c2ccc3c(c2)N2C[C@@H]4CC[C@H](COc5c(c(C(F)(F)F)nn5C)-c5cc(cc(C)n5)C(=O)/N=C/2N3)C4)CCN1]

The title compound was prepared in a manner similar to that in Intermediate 47 [M+H]⁺=639.3.

### Intermediate 51: (7¹R,7³S,E)-5⁶-((S)-3-(methoxymethyl)piperazin-1-yl)-1¹,2⁶,5⁵-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

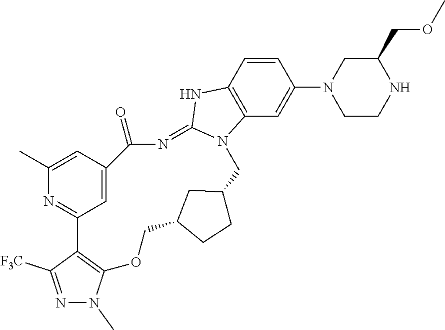 [SMILES: COC[C@@H]1CN(c2ccc3c(c2)N2C[C@@H]4CC[C@H](COc5c(c(C(F)(F)F)nn5C)-c5cc(cc(C)n5)C(=O)/N=C/2N3)C4)CCN1]

The title compound was prepared in a manner similar to that in Intermediates 1 and 2. [M+H]⁺=585.3.

### Intermediate 52: 3-((6-methyl-5-(4-oxopiperidin-1-yl)pyridin-2-yl)amino)piperidine-2,6-dione

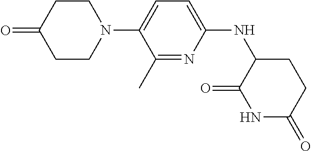 [SMILES: Cc1nc(NC2CCC(=O)NC2=O)ccc1N1CCC(=O)CC1]

The title compound was prepared in a manner similar to that in Intermediate 32. [M+H]⁺=317.2.

### Intermediate 53: (7¹R,7³S,E)-1³-(difluoromethyl)-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

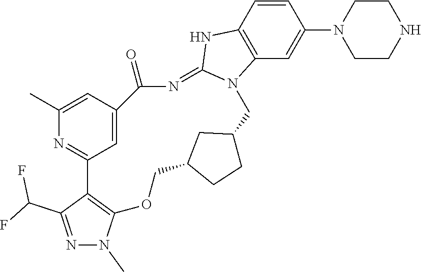 [SMILES: Cc1cc2cc(n1)-c1c(C(F)F)nn(C)c1OC[C@H]1CC[C@H](C1)CN1/C(=N/C2=O)Nc2ccc(N3CCNCC3)cc21]

The title compound was prepared in a manner similar to that in Intermediates 1 and 2. [M+H]⁺=577.3.

### Intermediate 54: (7¹R,7³S,E)-5⁵-fluoro-1¹,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

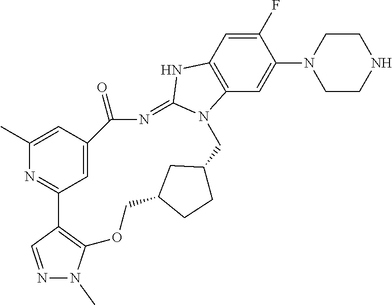 [SMILES: Cc1cc2cc(n1)-c1cnn(C)c1OC[C@H]1CC[C@H](C1)CN1/C(=N/C2=O)Nc2cc(F)c(N3CCNCC3)cc21]

The title compound was prepared in a manner similar to that in Intermediates 1 and 2. [M+H]⁺=545.3.

### Intermediate 55: 3-((3-chloro-4-(4-oxopiperidin-1-yl)phenyl)amino)piperidine-2,6-dione

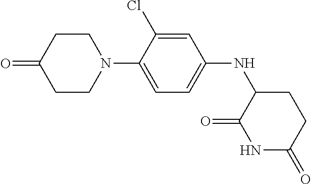 [SMILES: O=C1CCN(c2ccc(NC3CCC(=O)NC3=O)cc2Cl)CC1]

### Step 1: 8-(2-chloro-4-nitrophenyl)-1,4-dioxa-8-azaspiro[4.5]decane

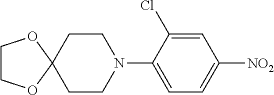 [SMILES: O=[N+]([O-])c1ccc(N2CCC3(CC2)OCCO3)c(Cl)c1]

To a stirred solution 2-chloro-1-fluoro-4-nitrobenzene (2 g, 11.5 mmol) in DMF (30 mL) were added 1,4-dioxa-8-azaspiro[4.5]decane (2.0 g, 13.8 mmol) and K₂CO₃ (3.2 g, 23 mmol). The resulting mixture was stirred for 12 hours at 80° C. The mixture was allowed to cool down to room temperature. The resulting mixture was poured into water (50 mL), filtered and the filter cake was washed with water to afford the product (3 g, 87%). [M+H]⁺=299.3.

### Step 2: 3-chloro-4-(1,4-dioxa-8-azaspiro[4.5]decan-8-yl)aniline

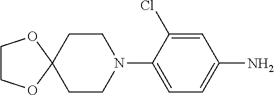 [SMILES: Nc1ccc(N2CCC3(CC2)OCCO3)c(Cl)c1]

To a stirred solution 8-(2-chloro-4-nitrophenyl)-1,4-dioxa-8-azaspiro[4.5]decane (3 g, 10.1 mmol) in THF (40 mL) were added Raney Ni (1 g). The resulting mixture was stirred for 1 hour at rt under H₂. The mixture was filtered and concentrated in vacuum to afford the product (2.6 g, 95%). [M+H]⁺=269.3.

### Step 3: 3-((3-chloro-4-(1,4-dioxa-8-azaspiro[4.5]decan-8-yl)phenyl)amino)piperidine-2,6-dione

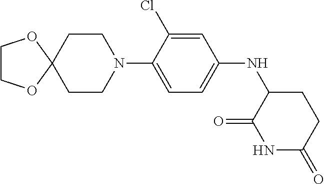 [SMILES: O=C1CCC(Nc2ccc(N3CCC4(CC3)OCCO4)c(Cl)c2)C(=O)N1]

To a solution of 3-chloro-4-(1,4-dioxa-8-azaspiro[4.5]decan-8-yl)aniline (2.6 g, 9.7 mmol) in DMF (40 mL) were added 3-bromopiperidine-2,6-dione (3.7 g, 19.4 mmol), and Na₂CO₃ (3.1 g, 29.1 mmol). The resulting mixture was stirred for 12 hours at 70° C. The mixture was poured into water and extracted with DCM (3×20 mL). The combined organic phase was washed with brine (1×15 mL), dried over Na₂SO₄, filtered and concentrated under reduced pressure to give the crude residue, which was purified by silica column chromatography (DCM:MeOH=100:1-10:1) to afford the product (1.8 g, 49%). [M+H]⁺=380.3.

### Step 4: 3-((3-chloro-4-(4-oxopiperidin-1-yl)phenyl)amino)piperidine-2,6-dione

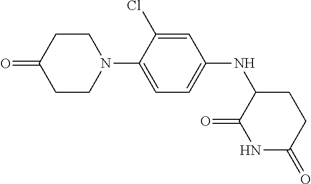 [SMILES: O=C1CCN(c2ccc(NC3CCC(=O)NC3=O)cc2Cl)CC1]

The solution of 3-((3-chloro-4-(1,4-dioxa-8-azaspiro[4.5]decan-8-yl)phenyl)amino)piperidine-2,6-dione (1 g, 2.6 mmol) in HCl (8 M, 10 mL) was stirred for 2 hours at rt. To the mixture was added sat. aq. NaHCO₃ dropwise and the pH was adjusted to 6-7. The liquid was extracted with DCM (30 mL×3). The combined organic phase was dried over anhydrous Na₂SO₄, filtered, and concentrated in vacuum to afford the title compound (800 mg, 92%). [M+H]⁺=336.2.

### Intermediate 56: 3-(5-((S)-3,3-difluoro-4-(piperazin-1-yl)piperidin-1-yl)-4-methylpyridin-2-yl)piperidine-2,6-dione

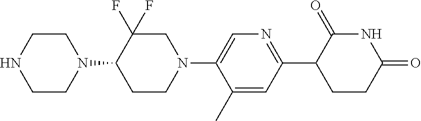 [SMILES: Cc1cc(C2CCC(=O)NC2=O)ncc1N1CC[C@H](N2CCNCC2)C(F)(F)C1]

The title compound was prepared in a procedure similar to that in intermediate 20. [M+H]⁺=408.2.

### Intermediate 57: (7¹R,7³S,E)-5⁶-bromo-1¹,2⁶,5⁵-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

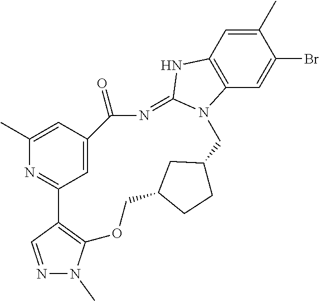 [SMILES: Cc1cc2cc(n1)-c1cnn(C)c1OC[C@H]1CC[C@H](C1)CN1/C(=N/C2=O)Nc2cc(C)c(Br)cc21]

The title compound was prepared in a procedure similar to that in intermediate 1. [M+H]⁺=535.2.

### Intermediate 58: (7¹R,7³S,E)-1³-(difluoromethyl)-5⁶-((R)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

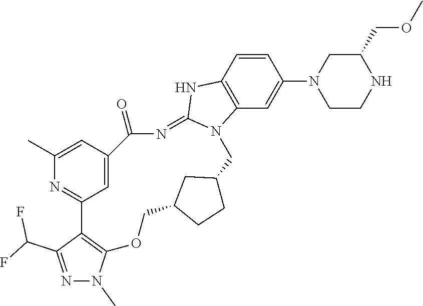 [SMILES: COC[C@H]1CN(c2ccc3c(c2)N2C[C@@H]4CC[C@H](COc5c(c(C(F)F)nn5C)-c5cc(cc(C)n5)C(=O)/N=C/2N3)C4)CCN1]

The title compound was prepared in a manner similar to that in Intermediates 1 and 2. [M+H]⁺=621.2.

### Intermediate 59: 3-(5-(4-(methoxymethyl)-4-(piperazin-1-yl)piperidin-1-yl)-6-methylpyridin-2-yl)piperidine-2,6-dione

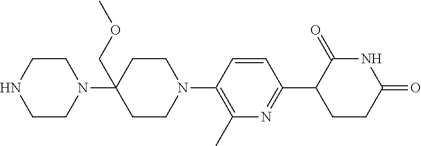 [SMILES: COCC1(N2CCNCC2)CCN(c2ccc(C3CCC(=O)NC3=O)nc2C)CC1]

The title compound was prepared in a manner similar to that in Intermediate 20. [M+H]⁺=416.5.

### Intermediate 61: (7¹R,7³S,E)-5⁶-((S)-4-(azetidin-3-yl)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

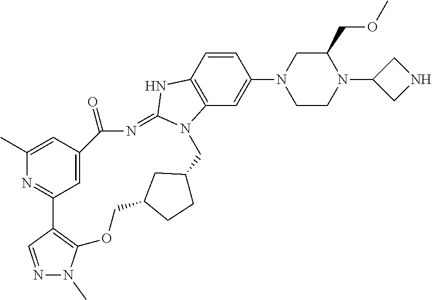 [SMILES: COC[C@@H]1CN(c2ccc3c(c2)N2C[C@@H]4CC[C@H](COc5c(cnn5C)-c5cc(cc(C)n5)C(=O)/N=C/2N3)C4)CCN1C1CNC1]

### Step 1: tert-butyl 3-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)azetidine-1-carboxylate

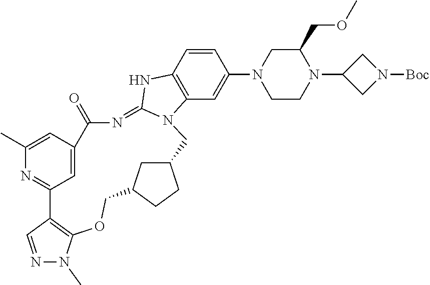

To a solution of intermediate 3 (80 mg, 0.14 mmol) and tert-butyl 3-oxoazetidine-1-carboxylate (50 mg, 0.28 mmol) in DCE (6 mL) was added STAB (89 mg, 0.42 mmol). Then the mixture was stirred at RT overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=20:1) to afford the title product (95 mg, 93.1%). [M+H]⁺=726.6.

### Step 2: (7¹R,7³S,E)-5⁶-((S)-4-(azetidin-3-yl)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

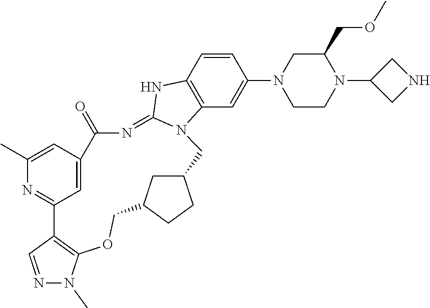

To a solution of tert-butyl 3-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)azetidine-1-carboxylate (95 mg, 0.13 mmol) in DCM (5 mL) was added TFA (1 mL). The mixture was stirred at room temperature for 60 minutes. The mixture was concentrated and basified with sat. aq. NaHCO₃. The liquid was extracted with DCM/MeOH (3×20 mL). The combined organic phase was dried over Na₂SO₄, filtered and concentrated in vacuum to afford the title product (78 mg, 95.1%). [M+H]⁺=626.6.

### Intermediate 62: 3-(3,5-difluoro-4-(4-oxopiperidin-1-yl)phenoxy)piperidine-2,6-dione

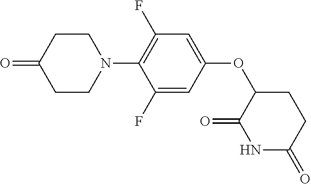 [SMILES: O=C1CCN(c2c(F)cc(OC3CCC(=O)NC3=O)cc2F)CC1]

The title compound was prepared in a manner similar to that in Intermediate 31. [M+H]⁺=339.3.

### Intermediate 63: 3-(4-(azetidin-3-ylamino)-2,6-difluorophenyl)piperidine-2,6-dione

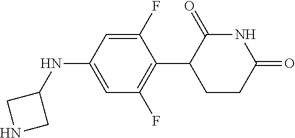 [SMILES: O=C1CCC(c2c(F)cc(NC3CNC3)cc2F)C(=O)N1]

### Step 1: tert-butyl 3-((4-(2,6-bis(benzyloxy)pyridin-3-yl)-3,5-difluorophenyl)amino)azetidine-1-carboxylate

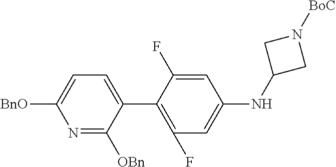

To the solution of intermediate 6 (3.0 g, 6.2 mmol), tert-butyl 3-aminoazetidine-1-carboxylate (2.1 g, 12.4 mmol) and Cs₂CO₃ (4.0 g, 12.4 mmol) in 80 mL 1,4-dioxane, were added Pd₂(dba)₃ (1.1 g, 1.24 mmol) and XantPhos (1.4 g, 2.48 mmol). The mixture was stirred at 100° C. for 16 hours under N₂. The mixture was filtered through a celite pad and washed with DCM (50 mL). The filtrate was concentrated under reduced pressure to give the crude residue, which was purified by silica gel column chromatography (PE:EA=100:1-2:1) to afford the product (3.4 g, 95.0%). [M+H]⁺=574.5.

### Step 2: tert-butyl 3-((4-(2,6-dioxopiperidin-3-yl)-3,5-difluorophenyl)amino)azetidine-1-carboxylate

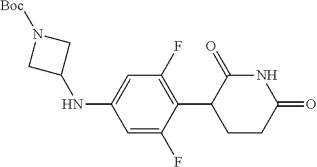 [SMILES: CC(C)(C)OC(=O)N1CC(Nc2cc(F)c(C3CCC(=O)NC3=O)c(F)c2)C1]

To the solution of tert-butyl 3-((4-(2,6-bis(benzyloxy)pyridin-3-yl)-3,5-difluorophenyl)amino)azetidine-1-carboxylate (3.4 g, 5.9 mmol) in 40 mL DMF and 40 mL iPrOH was added Pd/C (2.0 g, 10 wt. %, wet). The mixture was stirred at 50° C. for 48 hours under hydrogen atmosphere (balloon). The mixture was cooled to room temperature and filtered by celite directly. The filtrate was concentrated in vacuum to give the crude residue, which was purified by silica gel column chromatography (DCM:MeOH=100:0-20:1) to afford the product (1.8 g, 76.9%). [M+H]⁺=396.5.

### Step 3: 3-(4-(azetidin-3-ylamino)-2,6-difluorophenyl)piperidine-2,6-dione

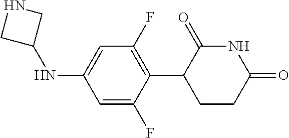 [SMILES: O=C1CCC(c2c(F)cc(NC3CNC3)cc2F)C(=O)N1]

To a solution of tert-butyl 3-((4-(2,6-dioxopiperidin-3-yl)-3,5-difluorophenyl)amino)azetidine-1-carboxylate (530 mg, 1.3 mmol) in DCM (6 mL) was added TFA (2 mL). The reaction was stirred at room temperature for 60 minutes. The mixture was concentrated in vacuum to afford the title product as TFA salt (395 mg, 99.5%). [M+H]⁺=296.5.

### Intermediate 64: (7¹R,7³S,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

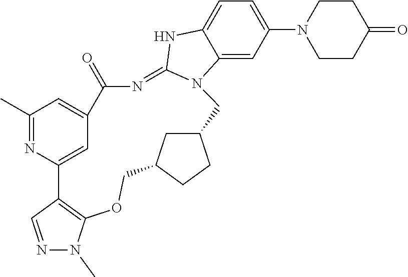 [SMILES: Cc1cc2cc(n1)-c1cnn(C)c1OC[C@H]1CC[C@H](C1)CN1/C(=N/C2=O)Nc2ccc(N3CCC(=O)CC3)cc21]

### Step 1: (7¹R,7³S,E)-1¹,2⁶-dimethyl-5⁶-(1,4-dioxa-8-azaspiro[4.5]decan-8-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

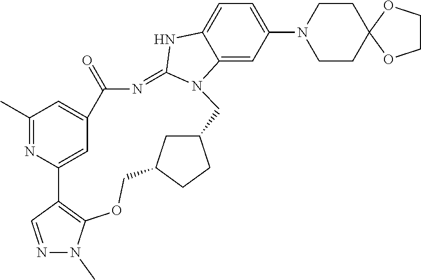 [SMILES: Cc1cc2cc(n1)-c1cnn(C)c1OC[C@H]1CC[C@H](C1)CN1/C(=N/C2=O)Nc2ccc(N3CCC4(CC3)OCCO4)cc21]

To the solution of intermediate 1 (1 g, 1.92 mmol), 1,4-dioxa-8-azaspiro[4.5]decane (549 mg, 3.84 mmol) and t-BuONa (369 mg, 3.84 mmol) in 20 mL DMA were added Pd₂(dba)₃ (348 mg, 0.38 mmol) and RuPhos (177 mg, 0.38 mmol). The mixture was stirred at 80° C. for 0.5 hour under N₂. The reaction was quenched with NH₄Cl/H₂O (15 mL) and extracted by DCM (3×15 mL). The combined organic phase was washed with brine (1×15 mL), dried over Na₂SO₄, filtered and concentrated in vacuum. The residue was purified by silica gel column (DCM:CH₃OH=15:1) to give the title product (1 g, 88.5%). [M+H]⁺=584.3.

### Step 2: (7¹R,7³S,E)-1¹,2⁶-dimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

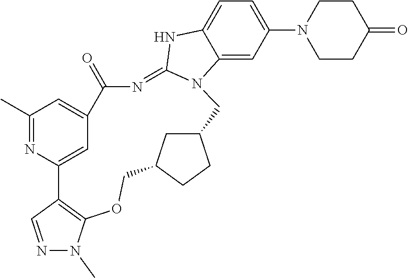 [SMILES: Cc1cc2cc(n1)-c1cnn(C)c1OC[C@H]1CC[C@H](C1)CN1/C(=N/C2=O)Nc2ccc(N3CCC(=O)CC3)cc21]

The solution of (7¹R,7³S,E)-1¹,2⁶-dimethyl-5⁶-(1,4-dioxa-8-azaspiro[4.5]decan-8-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one (1 g, 2.6 mmol) in HCl (8 M, 10 mL) was stirred for 2 hours at rt. To the mixture was added sat. aq. NaHCO₃ dropwise and the pH was adjusted to 6-7. The liquid was extracted with DCM (20 mL×3). The combined organic phase was dried over anhydrous Na₂SO₄, filtered and concentrated in vacuum to afford the title compound (800 mg, 87%). [M+H]⁺=540.3.

### Intermediate 65: (R)-3-(2,6-difluoro-4-(piperidin-4-yloxy)phenyl)piperidine-2,6-dione

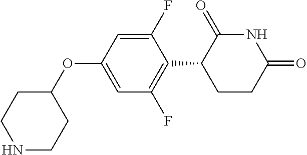 [SMILES: O=C1CC[C@H](c2c(F)cc(OC3CCNCC3)cc2F)C(=O)N1]

### Step 1: 2,6-bis(benzyloxy)-3-(2,6-difluoro-4-(4,4,5,5-tetramethyl-1,3,2-dioxaborolan-2-yl)phenyl)pyridine

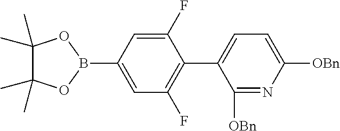 [SMILES: CC1(C)OB(c2cc(F)c(-c3ccc(OCc4ccccc4)nc3OCc3ccccc3)c(F)c2)OC1(C)C]

To a solution of intermediate 6 (35 g, 72.8 mmol) in dioxane (300 mL) were added B(pin)₂(37.0 g, 146 mmol), Pd(dppf)Cl₂ (5.28 g, 7.30 mmol), and K₂CO₃ (30.1 g, 218 mmol). The resulting solution was stirred overnight at 100° C. under N₂ atmosphere. After cooled to room temperature and filtered, the filter cake was washed with EA (100 mL). The filtrate was concentrated under reduced pressure. The crude product was purified by silica column chromatography (EA:PE=0-10%) to afford 2,6-bis(benzyloxy)-3-(2,6-difluoro-4-(4,4,5,5-tetramethyl-1,3,2-dioxaborolan-2-yl)phenyl)pyridine (30 g, 77.9%). [M+H]⁺=530.4.

### Step 2: 4-(2,6-bis(benzyloxy)pyridin-3-yl)-3,5-difluorophenol

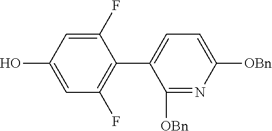 [SMILES: Oc1cc(F)c(-c2ccc(OCc3ccccc3)nc2OCc2ccccc2)c(F)c1]

To a stirred mixture of 2,6-bis(benzyloxy)-3-(2,6-difluoro-4-(4,4,5,5-tetramethyl-1,3,2-dioxaborolan-2-yl)phenyl)pyridine (30 g, 56.7 mmol) in AcOH (100 mL) and THF (100 mL) were added H₂O₂ (100 mL) in portions at 0° C. The mixture was stirred overnight at rt. Then sat. aq. Na₂S₂O₃ (150 mL) was added and then the mixture was extracted with EA (50 mL×3). The organic layer was washed with brine, dried over anhydrous Na₂SO₄. After filtration, the filtrate was concentrated under reduced pressure and purified by silica column chromatography (EA:PE=0-30%) to afford 4-(2,6-bis(benzyloxy)pyridin-3-yl)-3,5-difluorophenol (21 g, 88.48%). [M+H]⁺=420.43.

### Step 3: tert-butyl 4-(4-(2,6-bis(benzyloxy)pyridin-3-yl)-3,5-difluorophenoxy)piperidine-1-carboxylate

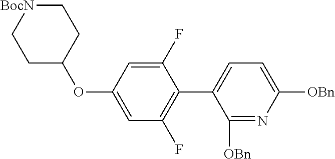 [SMILES: CC(C)(C)OC(=O)N1CCC(Oc2cc(F)c(-c3ccc(OCc4ccccc4)nc3OCc3ccccc3)c(F)c2)CC1]

A 500 mL round-bottomed flask equipped with a magnetic stirrer were charged with 4-(2,6-bis(benzyloxy)pyridin-3-yl)-3,5-difluorophenol (20.5 g, 48.9 mmol), tert-butyl 4-((methylsulfonyl)oxy)piperidine-1-carboxylate (20.5 g, 73.4 mmol), Cs₂CO₃ (47.8 g, 146.6 mmol), and DMF (210 mL). The resulting mixture was degassed under reduced pressure and purged with N₂ for three times, then stirred at 110° C. for 2 hours. After cooled to room temperature, the reaction was quenched with water (400 mL) and extracted with EtOAc (100 mL×3). The combined organic layers were washed with brine (100 mL×3), dried over anhydrous Na₂SO₄. After filtration, the filtrate was concentrated under reduced pressure. The residue was purified by column chromatography (EA/PE, 12-16%) to afford tert-butyl 4-(4-(2,6-bis(benzyloxy)pyridin-3-yl)-3,5-difluorophenoxy)piperidine-1-carboxylate (14 g, 47.6%). [M+H]⁺=603.3.

### Step 4: tert-butyl 4-(4-(2,6-dioxopiperidin-3-yl)-3,5-difluorophenoxy)piperidine-1-carboxylate

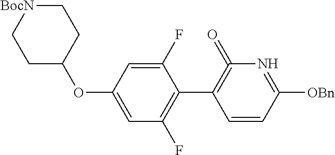

A 500 mL round-bottomed flask equipped with a magnetic stirrer, were charged with tert-butyl 4-(4-(2,6-bis(benzyloxy)pyridin-3-yl)-3,5-difluorophenoxy)piperidine-1-carboxylate (14 g, 23.3 mmol), dry THF (240 m1), and Pd/C (10 wt %, 27 g). The resulting mixture was degassed under reduced pressure and purged with H₂ for five times, then stirred overnight at 50° C. under H₂ atmosphere. The mixture was diluted with THF/DCM/MEOH (200 mL/200 mL/200 mL), then sonicated in an ultrasonic washer for 5 minutes, followed by filtration through a pad of celite. The filtrate was concentrated under vacuum. The residue was purified by column chromatography (EA/PE, 40-50%) to afford tert-butyl 4-(4-(2,6-dioxopiperidin-3-yl)-3,5-difluorophenoxy)piperidine-1-carboxylate (6.9 g, 70.0%). [M+H]⁺=425.3.

### Step 5: tert-butyl (R)-4-(4-(2,6-dioxopiperidin-3-yl)-3,5-difluorophenoxy)piperidine-1-carboxylate

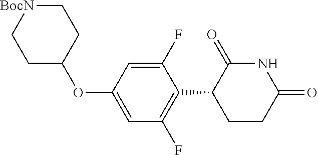 [SMILES: CC(C)(C)OC(=O)N1CCC(Oc2cc(F)c([C@H]3CCC(=O)NC3=O)c(F)c2)CC1]

The crude product (6.7 g) was purified by Prep-SFC with the following conditions (column: CHIRALPAK IC 5*25 cm, 5 m, Mobile phase A: CO₂, mobile phase B: MeOH (1% 2 mM NH₃-MeOH), Gradient A:B=40:60, Flow rate 130 mL/min, column temp 30° C.) to afford tert-butyl (R)-4-(4-(2,6-dioxopiperidin-3-yl)-3,5-difluorophenoxy)piperidine-1-carboxylate, which corresponds to peak A @ 8.45 min. [M+H]⁺=425.3.

### Step 6: (R)-3-(2,6-difluoro-4-(piperidin-4-yloxy)phenyl)piperidine-2,6-dione

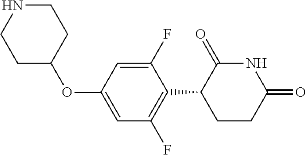 [SMILES: O=C1CC[C@H](c2c(F)cc(OC3CCNCC3)cc2F)C(=O)N1]

To a solution of tert-butyl (R)-4-(4-(2,6-dioxopiperidin-3-yl)-3,5-difluorophenoxy)piperidine-1-carboxylate (500 mg, 1.18 mmol) in DCM (10 mL) was added HCl (4 M in dioxane, 3 mL). The mixture was stirred in a flask at room temperature for 2 h. The mixture was evaporated in vacuum to afford the crude product (350 mg, 91.6%), which was used for next step without further purification. [M+H]⁺=325.2.

### Intermediate 66: 3-(5-(4-(hydroxymethyl)-4-(piperazin-1-yl)piperidin-1-yl)-6-methylpyridin-2-yl)piperidine-2,6-dione

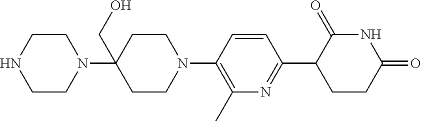 [SMILES: Cc1nc(C2CCC(=O)NC2=O)ccc1N1CCC(CO)(N2CCNCC2)CC1]

The title compound was prepared in a manner similar to that in Intermediate 20. [M+H]⁺=402.3.

### Intermediate 67: 3-(3-fluoro-4-((3S,4R)-3-fluoro-4-(piperazin-1-yl)piperidin-1-yl)phenoxy)piperidine-2,6-dione

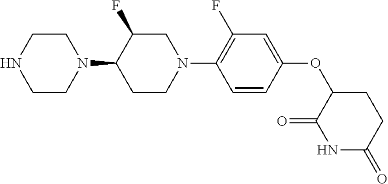 [SMILES: O=C1CCC(Oc2ccc(N3CC[C@@H](N4CCNCC4)[C@@H](F)C3)c(F)c2)C(=O)N1]

### Step 1: 1-benzyl-4-((3S,4R)-1-(4-(benzyloxy)-2-fluorophenyl)-3-fluoropiperidin-4-yl)piperazine

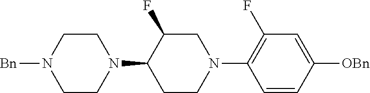 [SMILES: Fc1cc(OCc2ccccc2)ccc1N1CC[C@@H](N2CCN(Cc3ccccc3)CC2)[C@@H](F)C1]

To a stirred solution of 4-(benzyloxy)-1-bromo-2-fluorobenzene (1 g, 3.6 mmol) and 1-benzyl-4-((3S,4R)-3-fluoropiperidin-4-yl)piperazine (986 mg, 3.6 mmol, prepared in a manner similar to steps 1-2 in intermediate 20) in dioxane (20 mL) were added Cs₂CO₃ (2.2 g, 7.1 mmol), Ruphos (663 mg, 1.42 mmol) and Pd₂(dba)₃ (651 mg, 0.7 mmol) at room temperature under nitrogen atmosphere. The resulting mixture was stirred for 6 h at 100° C. under nitrogen atmosphere. The mixture was allowed to cool down to room temperature. The resulting mixture was concentrated under reduced pressure. The residue was diluted with EtOAc (100 mL), washed with water (3×50 mL) and brine (50 mL). The organic layer was dried over anhydrous Na₂SO₄, filtered and concentrated under reduced pressure. The residue was purified by silica gel column chromatography, eluted with DCM/MeOH (15:1) to afford the product (700 mg, 41.2%); [M+H]⁺=478.3.

### Step 2: tert-butyl 4-((3S,4R)-3-fluoro-1-(2-fluoro-4-hydroxyphenyl)piperidin-4-yl)piperazine-1-carboxylate

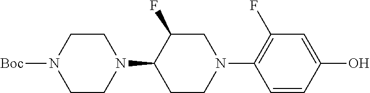 [SMILES: CC(C)(C)OC(=O)N1CCN([C@@H]2CCN(c3ccc(O)cc3F)C[C@@H]2F)CC1]

Under N₂, to a solution of 1-benzyl-4-((3S,4R)-1-(4-(benzyloxy)-2-fluorophenyl)-3-fluoropiperidin-4-yl)piperazine (700 mg, 1.5 mmol) and di-tert-butyl dicarbonate (640 mg, 2.9 mmol) in DMF (10 mL)/i-PrOH (10 mL) was added 10% Pd/C (700 mg) at 25° C. Then the mixture was exchanged with H₂ twice and stirred under H₂ atmosphere at 50° C. for 12 hours. The mixture was filtered through a pad of Celite and washed with MeOH (50 mL). The filtrate was concentrated under vacuum. The residue was purified by silica gel column chromatography, eluted with DCM/MeOH (20:1) to afford the product (460 mg, 79%); [M+H]⁺=398.4.

### Step 3: tert-butyl 4-((3S,4R)-1-(4-((2,6-dioxopiperidin-3-yl)oxy)-2-fluorophenyl)-3-fluoropiperidin-4-yl)piperazine-1-carboxylate

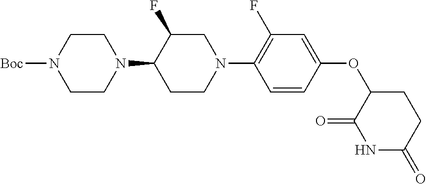 [SMILES: CC(C)(C)OC(=O)N1CCN([C@@H]2CCN(c3ccc(OC4CCC(=O)NC4=O)cc3F)C[C@@H]2F)CC1]

The solution of tert-butyl 4-((3S,4R)-3-fluoro-1-(2-fluoro-4-hydroxyphenyl)piperidin-4-yl)piperazine-1-carboxylate (450 mg, 1.1 mmol), 3-bromopiperidine-2,6-dione (434 mg, 2.3 mmol) and Cs₂CO₃ (1.1 g, 3.4 mmol) in DMSO (15 mL) was stirred at 50° C. for 5 hr and diluted with EtOAc (400 mL), washed with brine (3×150 mL). The organic layer was dried over anhydrous Na₂SO₄, filtered and concentrated under reduced pressure. The residue was purified by silica gel column chromatography, eluted with DCM/MeOH (10:1) to afford the product (210 mg, 36.5%); [M+H]⁺=509.6.

### Step 4: 3-(3-fluoro-4-((3S,4R)-3-fluoro-4-(piperazin-1-yl)piperidin-1-yl)phenoxy)piperidine-2,6-dione

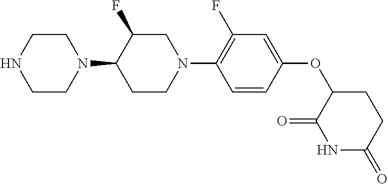 [SMILES: O=C1CCC(Oc2ccc(N3CC[C@@H](N4CCNCC4)[C@@H](F)C3)c(F)c2)C(=O)N1]

To a solution of tert-butyl 4-((3S,4R)-1-(4-((2,6-dioxopiperidin-3-yl)oxy)-2-fluorophenyl)-3-fluoropiperidin-4-yl)piperazine-1-carboxylate (210 mg, 0.4 mmol) in DCM (5 mL) was added TFA (2 mL). The reaction was stirred at room temperature for 2 hr and then concentrated in vacuo. The residue was dissolved in DCM (100 mL), washed with sat. aq. NaHCO₃ (3×50 mL) and brine (50 mL), dried over Na₂SO₄, filtered and concentrated under reduced pressure to afford the product (130 mg, 77%); [M+H]⁺=409.5.

### Intermediate 68: 3-(2,6-difluoro-4-(piperidin-4-ylamino)phenyl)piperidine-2,6-dione

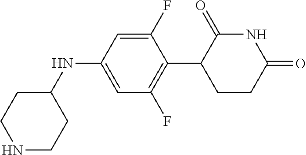 [SMILES: O=C1CCC(c2c(F)cc(NC3CCNCC3)cc2F)C(=O)N1]

The title compound was prepared in a manner similar to that in Intermediate 63. [M+H]⁺=324.2.

### Intermediate 69: (7¹R,7³S,E)-1¹,2⁶-dimethyl-5⁶-(2,6-diazaspiro[3.3]heptan-2-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

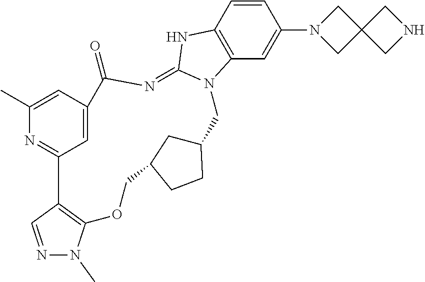 [SMILES: Cc1cc2cc(n1)-c1cnn(C)c1OC[C@H]1CC[C@H](C1)CN1/C(=N/C2=O)Nc2ccc(N3CC4(CNC4)C3)cc21]

The title compound was prepared in a manner similar to that in Intermediate 2. [M+H]⁺=539.5.

### Intermediate 70: 3-((3-methyl-4-(4-oxopiperidin-1-yl)phenyl)amino)piperidine-2,6-dione

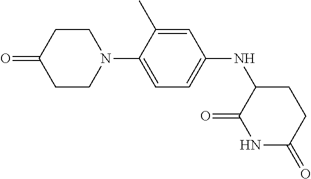 [SMILES: Cc1cc(NC2CCC(=O)NC2=O)ccc1N1CCC(=O)CC1]

### Step 1: 8-(2-methyl-4-nitrophenyl)-1,4-dioxa-8-azaspiro[4.5]decane

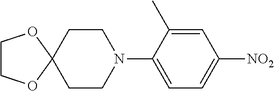 [SMILES: Cc1cc([N+](=O)[O-])ccc1N1CCC2(CC1)OCCO2]

To a stirred solution 1-fluoro-2-methyl-4-nitrobenzene (2 g, 12.9 mmol) in DMF (30 mL) were added 1,4-dioxa-8-azaspiro[4.5]decane (2.2 g, 15.5 mmol) and K₂CO₃ (3.6 g, 25.8 mmol). The resulting mixture was stirred for 12 hours at 80° C. The mixture was allowed to cool down to room temperature. The resulting mixture was poured into water (30 mL), filtered and the filter cake was washed with water (30 mL) to afford the product (3.2 g, 89%). [M+H]⁺=279.3.

### Step 2: 3-methyl-4-(1,4-dioxa-8-azaspiro[4.5]decan-8-yl)aniline

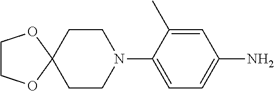 [SMILES: Cc1cc(N)ccc1N1CCC2(CC1)OCCO2]

To a stirred solution 8-(2-methyl-4-nitrophenyl)-1,4-dioxa-8-azaspiro[4.5]decane (3.2 g, 11.5 mmol) in THF (40 mL) was added Raney Ni (1 g). The resulting mixture was stirred for 1 hour at rt under H₂. The mixture was filtered and concentrated in vacuum to afford the product (2.7 g, 95%). [M+H]⁺=249.3.

### Step 3: 2,6-bis(benzyloxy)-N-(3-methyl-4-(1,4-dioxa-8-azaspiro[4.5]decan-8-yl)phenyl)pyridin-3-amine

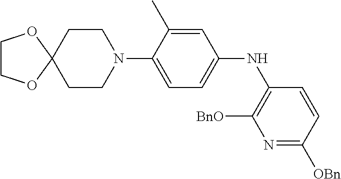 [SMILES: Cc1cc(Nc2ccc(OCc3ccccc3)nc2OCc2ccccc2)ccc1N1CCC2(CC1)OCCO2]

To the solution of 3-methyl-4-(1,4-dioxa-8-azaspiro[4.5]decan-8-yl)aniline (2 g, 8.1 mmol) in dioxane (40 mL) were added 2,6-bis(benzyloxy)-3-bromopyridine (3.3 g, 8.9 mmol), Pd₂(dba)₃ (1.5 g, 1.6 mmol), RuPhos (745 mg, 1.6 mmol) and Cs₂CO₃ (5.3 g, 16.2 mmol). The mixture was stirred at 100° C. for 12 hours under N₂. The mixture was diluted with water (60 mL) and extracted by EtOAc (40 mL×3). The organic layer was washed with brine (30 mL×3), dried over anhydrous Na₂SO₄, filtered and concentrated under reduced pressure. The resulting mixture was purified by silica column chromatography (PE:EA=10:1-5:1) to afford the product (3.5 g, 80%). [M+H]⁺=538.4.

### Step 4: 3-((3-methyl-4-(1,4-dioxa-8-azaspiro[4.5]decan-8-yl)phenyl)amino)piperidine-2,6-dione

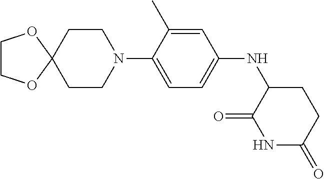 [SMILES: Cc1cc(NC2CCC(=O)NC2=O)ccc1N1CCC2(CC1)OCCO2]

To a stirred solution 2,6-bis(benzyloxy)-N-(3-methyl-4-(1,4-dioxa-8-azaspiro[4.5]decan-8-yl)phenyl)pyridin-3-amine (3.5 g, 6.5 mmol) in DMF/i-PrOH (100 mL/40 mL) were added Pd/C (3.5 g, 10 wt %, wet). The resulting mixture was stirred for 16 hour at 50° C. under H₂. The mixture was filtered and concentrated in vacuum to afford the product (2.0 g, 86%). [M+H]⁺=360.3.

### Step 5: 3-((3-methyl-4-(4-oxopiperidin-1-yl)phenyl)amino)piperidine-2,6-dione

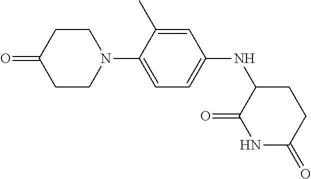 [SMILES: Cc1cc(NC2CCC(=O)NC2=O)ccc1N1CCC(=O)CC1]

The solution of 3-((3-methyl-4-(1,4-dioxa-8-azaspiro[4.5]decan-8-yl)phenyl)amino)piperidine-2,6-dione (1 g, 2.8 mmol) in HCl (8 M, 10 mL) was stirred for 2 hours at rt. To the mixture was added sat. aq. NaHCO₃ dropwise and the pH was adjusted to 6-7. The liquid was extracted with DCM (20 mL×3). The combined organic phase was dried over anhydrous Na₂SO₄, filtered and concentrated in vacuum to afford the title compound (800 mg, 89%). [M+H]⁺=316.2.

### Intermediate 71: (7¹R,7³S,E)-1¹,2⁶-dimethyl-5⁶-((S)-3-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

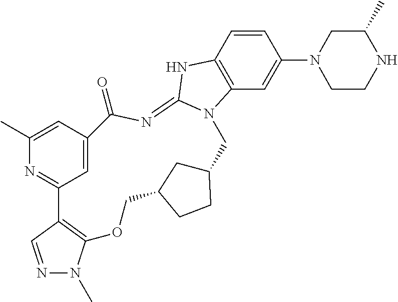

The title compound was prepared in a manner similar to that in Intermediate 2. [M+H]⁺=541.2.

### Intermediate 72: (7¹R,7³S,E)-1¹,2⁶-dimethyl-5⁶-(2,7-diazaspiro[3.5]nonan-7-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

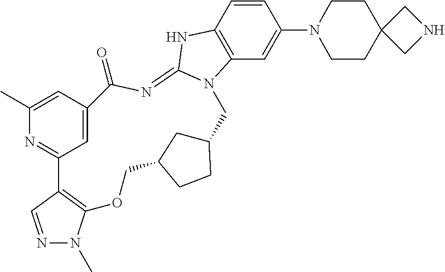 [SMILES: Cc1cc2cc(n1)-c1cnn(C)c1OC[C@H]1CC[C@H](C1)CN1/C(=N/C2=O)Nc2ccc(N3CCC4(CC3)CNC4)cc21]

The title compound was prepared in a manner similar to that in Intermediate 2. [M+H]⁺=567.5.

### Intermediate 73: (7¹R,7³S,E)-5⁶-((S)-4-(azetidin-3-yl)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

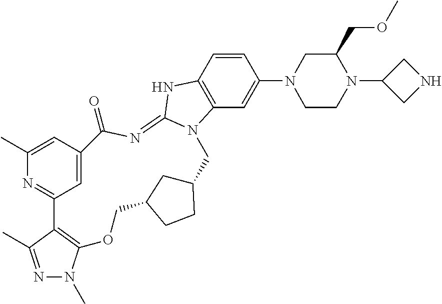 [SMILES: COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CNC1]

The title compound was prepared in a manner similar to that in Intermediate 61. [M+H]⁺=640.5.

### Intermediate 74: methyl 2-(1-(2-(benzyloxy)ethyl)-5-hydroxy-1H-pyrazol-4-yl)-6-methylisonicotinate

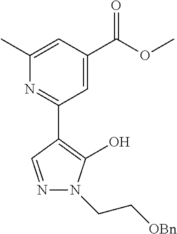

### Step 1: (2-(benzyloxy)ethyl)hydrazine

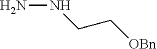 [SMILES: NNCCOCc1ccccc1]

To a stirred mixture of ((2-bromoethoxy)methyl)benzene (10 g, 46.7 mmol) in EtOH (100 mL) was added hydrazine hydrate (58 mL, 467 mmol) dropwise at 0° C. The resulting mixture was stirred overnight at rt. The resulting mixture was concentrated under reduced pressure, and extracted with EA. The combined organic layers were washed with brine, and dried over anhydrous Na₂SO₄. The crude product (10 g) was used in the next step directly without further purification. [M+H]⁺=167.1.

### Step 2: methyl 2-(4-bromo-6-methylpyridin-2-yl)acetate

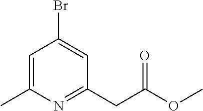

To a stirred mixture of 4-bromo-2,6-dimethylpyridine (20 g, 108.1 mmol) in THF (200 mL) was added LDA (108.1 mL, 2 M in THF) dropwise at −78° C. The resulting mixture was stirred for 30 min at −78° C. under N₂ atmosphere. To the above mixture was added dimethyl carbonate (9.73 g, 108.1 mmol) dropwise over 10 min at −78° C. The resulting mixture was stirred for additional 2 h at rt. The reaction mixture was quenched with NH₄Cl (aq.), and extracted with EA. The combined organic layers were washed with brine, dried over anhydrous Na₂SO₄. After filtration, the filtrate was concentrated under reduced pressure. The residue was purified by silica gel column chromatography, eluted with PE/EA (3:1) to afford methyl 2-(4-bromo-6-methylpyridin-2-yl)acetate (9 g, 34%). [M+H]⁺=244.1.

### Step 3: methyl (Z)-2-(4-bromo-6-methylpyridin-2-yl)-3-(dimethylamino)acrylate

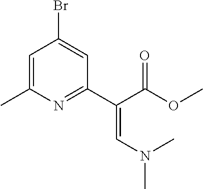

A mixture of methyl 2-(4-bromo-6-methylpyridin-2-yl)acetate (9 g, 37.0 mmol) and 1,1-dimethoxy-N,N-dimethylmethanamine (5.29 g, 44.4 mmol) in DMF (100 mL) was stirred overnight at 80° C. The resulting mixture was concentrated under reduced pressure. The crude product (11 g) was used in the next step directly without further purification. [M+H]⁺=299.2.

### Step 4: 1-(2-(benzyloxy)ethyl)-4-(4-bromo-6-methylpyridin-2-yl)-1H-pyrazol-5-ol

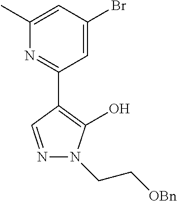 [SMILES: Cc1cc(Br)cc(-c2cnn(CCOCc3ccccc3)c2O)n1]

A mixture of methyl (Z)-2-(4-bromo-6-methylpyridin-2-yl)-3-(dimethylamino)acrylate (11 g crude, 36.9 mmol) and (2-(benzyloxy)ethyl)hydrazine (9.1 g crude, 55.35 mmol) in MeOH was stirred overnight at rt. The mixture was concentrated under reduced pressure. The residue was purified by silica gel column chromatography, eluted with PE/EA (5:1) to afford 1-(2-(benzyloxy)ethyl)-4-(4-bromo-6-methylpyridin-2-yl)-1H-pyrazol-5-ol (3.8 g, 27.1%). [M+H]⁺=388.3.

### Step 5: methyl 2-(1-(2-(benzyloxy)ethyl)-5-hydroxy-1H-pyrazol-4-yl)-6-methylisonicotinate

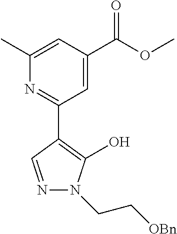

A mixture of 1-(2-(benzyloxy)ethyl)-4-(4-bromo-6-methylpyridin-2-yl)-1H-pyrazol-5-ol (3.4 g, 8.78 mmol), Pd(dppf)Cl₂ (0.72 g, 0.88 mmol) and DIEA (5.65 g, 43.8 mmol) in MeOH (30 mL) was stirred for 3 h at 100° C. under CO (20 atm.) atmosphere. The mixture was allowed to cool down to room temperature and concentrated under reduced pressure. The residue was purified by silica gel column chromatography, eluted with PE/EA (3:1) to afford methyl 2-(1-(2-(benzyloxy)ethyl)-5-hydroxy-1H-pyrazol-4-yl)-6-methylisonicotinate (3 g, 93.8%). [M+H]⁺=368.2. ¹H NMR (400 MHz, DMSO) δ 7.99 (s, 1H), 7.91 (s, 1H), 7.52 (s, 1H), 7.35-7.21 (m, 5H), 4.47 (s, 2H), 4.04 (t, J=4 Hz, 2H), 3.91 (s, 3H), 3.73 (t, J=4 Hz, 2H), 2.56 (s, 3H).

### Intermediate 75: (7¹R,7³S,E)-1¹-(2-(benzyloxy)ethyl)-5⁶-bromo-2⁶-methyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

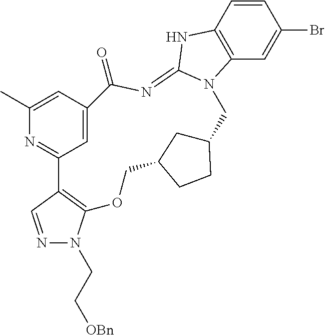 [SMILES: Cc1cc2cc(n1)-c1cnn(CCOCc3ccccc3)c1OC[C@H]1CC[C@H](C1)CN1/C(=N/C2=O)Nc2ccc(Br)cc21]

The title compound was prepared in a manner similar to that in Intermediate 1 with intermediate 74.

### Intermediate 76: (7¹R,7³S,E)-1¹-(2-hydroxyethyl)-2⁶-methyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

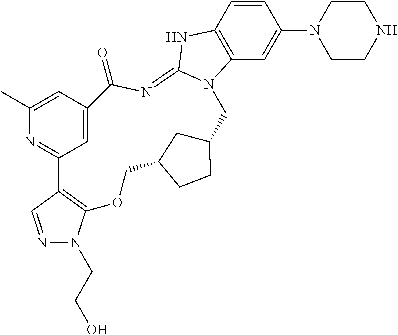 [SMILES: Cc1cc2cc(n1)-c1cnn(CCO)c1OC[C@H]1CC[C@H](C1)CN1/C(=N/C2=O)Nc2ccc(N3CCNCC3)cc21]

### Step 1: tert-butyl 4-((7¹R,7³S,E)-1-(2-(benzyloxy)ethyl)-2⁶-methyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-vl)piperazine-1-carboxylate

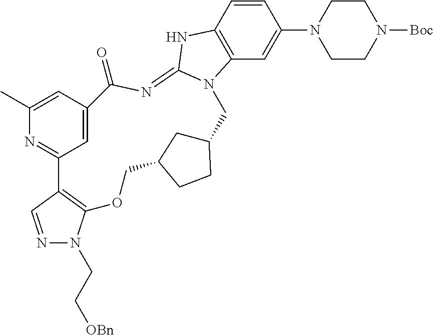 [SMILES: Cc1cc2cc(n1)-c1cnn(CCOCc3ccccc3)c1OC[C@H]1CC[C@H](C1)CN1/C(=N/C2=O)Nc2ccc(N3CCN(C(=O)OC(C)(C)C)CC3)cc21]

To the solution of intermediate 75 (300 mg, 0.47 mmol), tert-butyl piperazine-1-carboxylate (175 mg, 0.94 mmol) and t-BuONa (135 mg, 1.41 mmol) in 8 mL DMA were added Pd₂(dba)₃ (86 mg, 0.09 mmol) and RuPhos (84 mg, 0.18 mmol). The resulting mixture was stirred at 90° C. for 1 hour under N₂. The mixture was concentrated under reduced pressure to give the crude residue, which was purified by silica gel column chromatography (DCM:MeOH=100:1-20:1) to afford the product (265 mg, 75.7%). [M+H]⁺=747.5.

### Step 2: (7¹R,7³S,E)-1¹-(2-hydroxyethyl)-2⁶-methyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

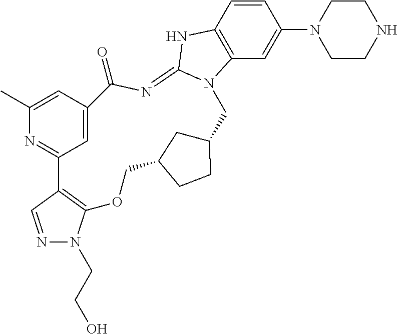 [SMILES: Cc1cc2cc(n1)-c1cnn(CCO)c1OC[C@H]1CC[C@H](C1)CN1/C(=N/C2=O)Nc2ccc(N3CCNCC3)cc21]

To the solution of tert-butyl 4-((7¹R,7³S,E)-1¹-(2-(benzyloxy)ethyl)-2⁶-methyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazine-1-carboxylate (265 mg, 0.35 mmol) in 10 mL DCM was added BBr₃ in DCM (1 M, 1.0 mL). After stirring at room temperature for 1 hour, the reaction mixture was quenched with aq. NaHCO₃, extracted with DCM (3×30 mL). The combined organic phase was dried over Na₂SO₄, filtered and concentrated in vacuum to give the crude residue, which was purified by silica gel column chromatography (DCM:MeOH=100:1-5:1) to afford the product (90 mg, 45.7%). [M+H]⁺=557.5.

### Intermediate 77: (7¹R,7³S,E)-1¹,2⁶-dimethyl-5⁶-(3-(methylamino)azetidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

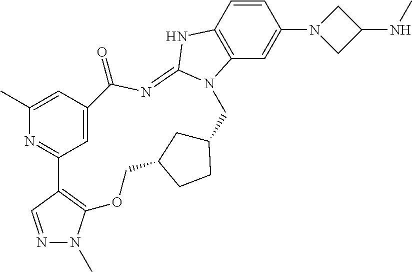

The title compound was prepared in a manner similar to that in Intermediate 2. [M+H]⁺=527.5.

### Intermediate 78: (7¹R,7³S,E)-1¹,2⁶-dimethyl-5⁶-(2,7-diazaspiro[3.5]nonan-2-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

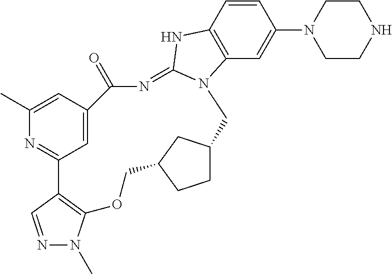 [SMILES: Cc1cc2cc(n1)-c1cnn(C)c1OC[C@H]1CC[C@H](C1)CN1/C(=N/C2=O)Nc2ccc(N3CCNCC3)cc21]

The title compound was prepared in a manner similar to that in Intermediate 2. [M+H]⁺=567.5

### Intermediate 79: (R)-3-(2,6-difluoro-4-(3-oxoazetidin-1-yl)phenyl)piperidine-2,6-dione

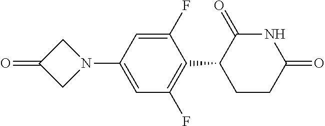 [SMILES: O=C1CN(c2cc(F)c([C@H]3CCC(=O)NC3=O)c(F)c2)C1]

### Step 1:2,6-bis(benzyloxy)-3-(4-(3-(benzyloxy)azetidin-1-yl)-2,6-difluorophenyl)pyridine

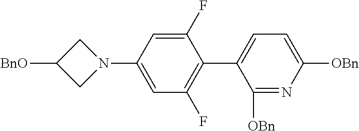 [SMILES: Fc1cc(N2CC(OCc3ccccc3)C2)cc(F)c1-c1ccc(OCc2ccccc2)nc1OCc1ccccc1]

A solution of intermediate 6 (20 g, 41.49 mmol), 3-(benzyloxy)azetidine hydrochloride (9.96 g, 49.79 mmol), Pd₂(dba)₃(3.79 g, 4.15 mmol), RuPhos (3.88 g, 8.3 mmol) and Cs₂CO₃ (40.58 g, 124.47 mmol) in dioxane (400 mL) was stirred at 100° C. for 3 h under nitrogen atmosphere. After cooled to room temperature, the reaction was quenched with water and extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (10:1) to afford 2,6-bis(benzyloxy)-3-(4-(3-(benzyloxy)azetidin-1-yl)-2,6-difluorophenyl)pyridine (17 g, 72.6%). [M+H]⁺=565.6.

### Step 2: 3-(2,6-difluoro-4-(3-hydroxyazetidin-1-yl)phenyl)piperidine-2,6-dione

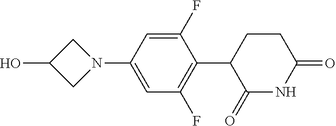 [SMILES: O=C1CCC(c2c(F)cc(N3CC(O)C3)cc2F)C(=O)N1]

To a solution of 2,6-bis(benzyloxy)-3-(4-(3-(benzyloxy)azetidin-1-yl)-2,6-difluorophenyl)pyridine (17 g, 30.09 mmol) in DCM/THF (200 mL/200 mL), was added Pd/C (10 wt %, wet, 34 g). The mixture was stirred for 2 days at 65° C. under hydrogen atmosphere. After cooled to room temperature. The resulting mixture was filtered, the filter cake was washed with iPrOH. The filtrate was concentrated under reduced pressure and purified by trituration with DCM/MeOH (10/1) to afford 3-(2,6-difluoro-4-(3-hydroxyazetidin-1-yl)phenyl)piperidine-2,6-dione (8 g, 89.9%). [M+H]⁺=297.1

### Step 3: 3-(4-(3-((tert-butyldimethylsilyl)oxy)azetidin-1-yl)-2,6-difluorophenyl)piperidine-2,6-dione

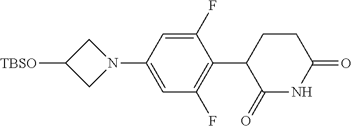 [SMILES: CC(C)(C)[Si](C)(C)OC1CN(c2cc(F)c(C3CCC(=O)NC3=O)c(F)c2)C1]

The solution of 3-(2,6-difluoro-4-(3-hydroxyazetidin-1-yl)phenyl)piperidine-2,6-dione (11 g, 37.16 mmol), TBSCl (11.15 g, 74.32 mmol) and imidazole (7.58 g, 111.49 mmol) in DMF (200 mL) was stirred for 2 h at rt. The resulting mixture was diluted with sat. aq. NaHCO₃ (500 mL) and extracted with EA (200 mL×3). The combined organic layers were washed with brine, dried over anhydrous Na₂SO₄. After filtration, the filtrate was concentrated under reduced pressure. The residue was purified by silica gel column chromatography, eluted with PE/EA (10:1) to afford 3-(4-(3-((tert-butyldimethylsilyl)oxy)azetidin-1-yl)-2,6-difluorophenyl)piperidine-2,6-dione (11 g, 72.4%). [M+H]⁺=411.2

### Step 4: (R)-3-(4-(3-((tert-butyldimethylsilyl)oxy)azetidin-1-yl)-2,6-difluorophenyl)piperidine-2,6-dione

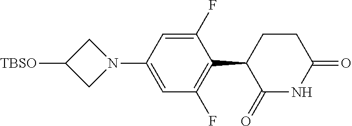 [SMILES: CC(C)(C)[Si](C)(C)OC1CN(c2cc(F)c([C@H]3CCC(=O)NC3=O)c(F)c2)C1]

The crude product (11 g) was purified by Prep-SFC and the title compound corresponds to (CHIRALPAK IF-3, 4.6*50 mm, 3 μm, MtBE (0.1% DEA):(MeOH:DCM=1:1)=80:20, peak A @1.084 min). (4 g, 36%, ee=100%). [M+H]⁺=411.2

### Step 5: (R)-3-(2,6-difluoro-4-(3-hydroxyazetidin-1-yl)phenyl)piperidine-2,6-dione

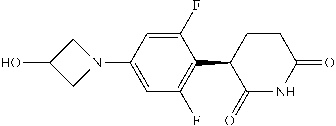 [SMILES: O=C1CC[C@H](c2c(F)cc(N3CC(O)C3)cc2F)C(=O)N1]

(R)-3-(4-(3-((tert-butyldimethylsilyl)oxy)azetidin-1-yl)-2,6-difluorophenyl)piperidine-2,6-dione (2.6 g, 6.3 mmol) was placed in a 250 mL round bottom flask with a magnetic stir bar. Then, 60 mL THF and 60 mL 1M HCl were added at 0° C. The mixture was stirred at room temperature for 3 hours. The mixture was added dropwise to sat. aq. NaHCO₃ and pH was adjusted to 6-7. The liquid was extracted with DCM (50 mL×3) and separated. The combined organic phase was concentrated in vacuum and purified with combiflash (DCM:MeOH=20:1) to afford the title compound (1.8 g, 95% yield). [M+H]⁺=297.2.

### Step 6: (R)-3-(2,6-difluoro-4-(3-oxoazetidin-1-yl)phenyl)piperidine-2,6-dione

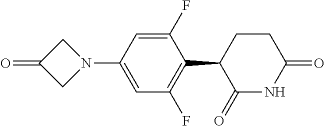 [SMILES: O=C1CN(c2cc(F)c([C@H]3CCC(=O)NC3=O)c(F)c2)C1]

To a stirred solution of (R)-3-(2,6-difluoro-4-(3-hydroxyazetidin-1-yl)phenyl)piperidine-2,6-dione (1.8 g, 6.0 mmol) in DCM (60 mL) were added Dess-Martin periodinane (3.8 g, 9.0 mmol) at room temperature. The resulting mixture was stirred for 2 h at room temperature. Then the mixture was diluted with DCM (100 mL), washed with sat. aq. Na₂S₂O₃ (100 mL), sat. aq. NaHCO₃ (3×100 mL) and brine (100 mL). The organic layer was dried over anhydrous Na₂SO₄, filtered and concentrated under reduced pressure to give the crude product (2.0 g), which was used without further purification. [M+H]⁺=295.2.

### Intermediate 80: (R)-1-(4-(2,6-dioxopiperidin-3-yl)-3,5-difluorophenyl)piperidine-4-carbaldehyde

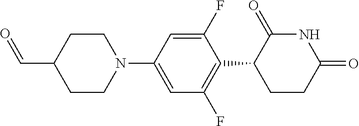 [SMILES: O=CC1CCN(c2cc(F)c([C@H]3CCC(=O)NC3=O)c(F)c2)CC1]

The title compound was prepared in a manner similar to that in Intermediate 7. [M+H]⁺=337.2

### Intermediate 82: 1-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidine-4-carbaldehyde

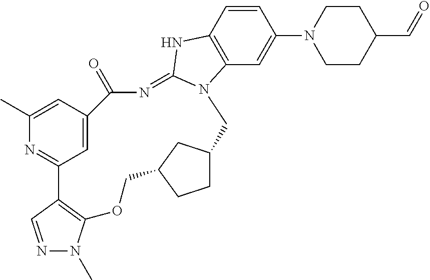 [SMILES: Cc1cc2cc(n1)-c1cnn(C)c1OC[C@H]1CC[C@H](C1)CN1/C(=N/C2=O)Nc2ccc(N3CCC(C=O)CC3)cc21]

The title compound was prepared in a manner similar to that in Intermediate 64. [M+H]⁺=554.5

### Intermediate 83: 3-((3,5-difluoro-4-(4-oxopiperidin-1-yl)phenyl)amino)piperidine-2,6-dione

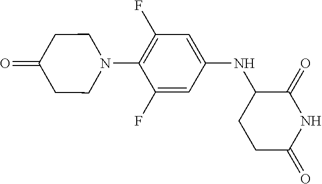 [SMILES: O=C1CCN(c2c(F)cc(NC3CCC(=O)NC3=O)cc2F)CC1]

The title compound was prepared in a manner similar to that in Intermediate 32. [M+H]⁺=338.2

### Intermediate 84: (7¹R,7³S,E)-1¹,1³,2⁶-trimethyl-5⁶-((R)-3-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

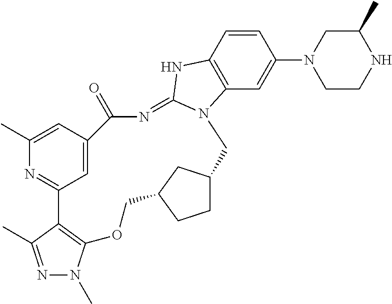

The title compound was prepared in a manner similar to that in intermediate 2 with intermediate 39. [M+H]+=555.3

### Intermediate 85: (7¹R,7³S,E)-1¹,1³,2⁶-trimethyl-5⁶-((S)-3-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

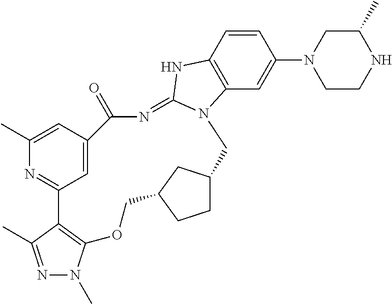

The title compound was prepared in a manner similar to that in intermediate 2 with intermediate 39. [M+H]+=555.3

### Intermediate 86: (7¹R,7³S,E)-1¹,1³,2⁶-trimethyl-5⁶-(4-(methylamino)piperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

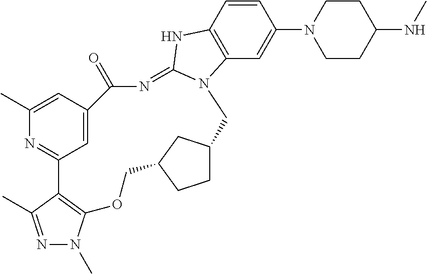

The title compound was prepared in a manner similar to that in intermediate 2 with intermediate 39. [M+H]+=569.3

### Intermediate 87: 3-((6-methyl-5-(4-oxopiperidin-1-yl)pyridin-2-yl)oxy)piperidine-2,6-dione

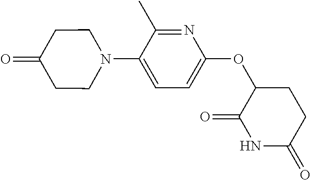 [SMILES: Cc1nc(OC2CCC(=O)NC2=O)ccc1N1CCC(=O)CC1]

### Step 1: 2,6-bis(benzyloxy)pyridin-3-ol

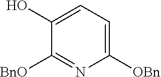 [SMILES: Oc1ccc(OCc2ccccc2)nc1OCc1ccccc1]

To a solution of 2,6-bis(benzyloxy)-3-(4,4,5,5-tetramethyl-1,3,2-dioxaborolan-2-yl)pyridine (20.0 g, 47.96 mmoL) and NaOH (3.8 g, 95.92 mmoL) in DCM (200 mL) was added H₂O₂ (40 mL). The reaction mixture was stirred for 2 h at rt. The reaction mixture was concentrated under reduced pressure. The residue was purified by silica gel column chromatography (PE/EA=0-6%) to afford 2,6-bis(benzyloxy)pyridin-3-ol (10.0 g, 68.02% yield). [M+H]⁺=308.12

### Step 2: 2,6-bis(benzyloxy)-3-((5-bromo-6-methylpyridin-2-yl)oxy)pyridine

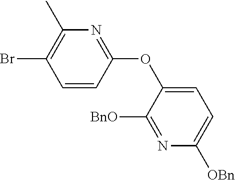 [SMILES: Cc1nc(Oc2ccc(OCc3ccccc3)nc2OCc2ccccc2)ccc1Br]

To a solution of 2,6-bis(benzyloxy)pyridin-3-ol (10.0 g, 32.57 mmoL) and 3-bromo-6-fluoro-2-methylpyridine (6.1 g, 32.57 mmoL) in DMSO (100 mL) was added Cs₂CO₃ (31.7 g, 97.71 mmoL). The reaction mixture was stirred for overnight at 80° C. The reaction mixture was diluted with H₂O and extracted with EtOAc. The combined organic layers were washed with brine, dried over anhydrous Na₂SO₄, filtered and concentrated under reduced pressure. The residue was purified by silica gel column chromatography (EA/PE=0-3%) to afford 2,6-bis(benzyloxy)-3-((5-bromo-6-methylpyridin-2-yl)oxy)pyridine (13.0 g, 83.87% yield). [M+H]⁺=477.07

### Step 3: 8-(6-((2,6-bis(benzyloxy)pyridin-3-yl)oxy)-2-methylpyridin-3-yl)-1,4-dioxa-8-azaspiro[4.5]decane

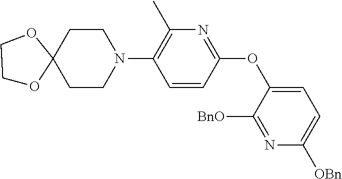 [SMILES: Cc1nc(Oc2ccc(OCc3ccccc3)nc2OCc2ccccc2)ccc1N1CCC2(CC1)OCCO2]

A mixture of 2,6-bis(benzyloxy)-3-((5-bromo-6-methylpyridin-2-yl)oxy)pyridine (13 g, 27.31 mmol), 1,4-dioxa-8-azaspiro[4.5]decane (5.85 g, 41.0 mmoL), Pd₂(dba)₃ (2.49 g, 2.73 mmol), Ruphos (2.59 g, 5.46 mmoL) and Cs₂CO₃ (26.6 g, 81.9 mmoL) in dioxane (130 mL) was stirred at 100° C. for overnight under nitrogen atmosphere. The reaction mixture was diluted with H₂O and extracted with EtOAc. The combined organic layers were washed with brine, dried over anhydrous Na₂SO₄, filtered and concentrated under reduced pressure. The residue was purified by silica gel column chromatography (EA/PE=0-20%) to afford 8-(6-((2,6-bis(benzyloxy)pyridin-3-yl)oxy)-2-methylpyridin-3-yl)-1,4-dioxa-8-azaspiro[4.5]decane (6.0 g, 40.8% yield). [M+H]⁺=540.24

### Step 4: 6-methyl-5-(1,4-dioxa-8-azaspiro[4.5]decan-8-yl)pyridin-2-ol

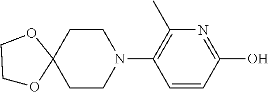 [SMILES: Cc1nc(O)ccc1N1CCC2(CC1)OCCO2]

A 250 mL round bottom flask equipped with a magnetic stirrer, were charged with 8-(6-((2,6-bis(benzyloxy)pyridin-3-yl)oxy)-2-methylpyridin-3-yl)-1,4-dioxa-8-azaspiro[4.5]decane (6.0 g, 11.1 mmol), THF (60.0 mL) and Pd/C (60 wt %, 6.0 g). The resulting mixture was degassed under reduced pressure and purged with H₂ for five times, then stirred at 50° C. for overnight. The mixture was filtration through a pad of celite. The filtrate was concentrated under reduced pressure. The residue was purified by silica gel column chromatography (MeOH/DCM=0-10%) to afford 6-methyl-5-(1,4-dioxa-8-azaspiro[4.5]decan-8-yl)pyridin-2-ol (2.1 g, 75.5%). [M+H]⁺=251.13

### Step 5: 3-((6-methyl-5-(1,4-dioxa-8-azaspiro[4.5]decan-8-yl)pyridin-2-yl)oxy)piperidine-2,6-dione

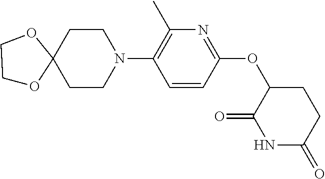 [SMILES: Cc1nc(OC2CCC(=O)NC2=O)ccc1N1CCC2(CC1)OCCO2]

To a solution of 6-methyl-5-(1,4-dioxa-8-azaspiro[4.5]decan-8-yl)pyridin-2-ol (2.1 g, 8.39 mmol) and 3-bromopiperidine-2,6-dione (2.0 g, 10.5 mmoL) in THF (21 mL) was added NaH (671.6 mg, 16.78 mmoL). The reaction mixture was stirred for overnight at rt. The reaction mixture was diluted with aq. sat. NH₄Cl and extracted with EtOAc. The combined organic layers were washed with brine, dried over anhydrous Na₂SO₄, filtered and concentrated under reduced pressure. The residue was purified by silica gel column chromatography (EA/PE=0-60%) to afford 3-((6-methyl-5-(1,4-dioxa-8-azaspiro[4.5]decan-8-yl)pyridin-2-yl)oxy)piperidine-2,6-dione (1.25 g, 41.6%). [M+H]⁺=362.2

### Step 6: 3-((6-methyl-5-(4-oxopiperidin-1-yl)pyridin-2-yl)oxy)piperidine-2,6-dione

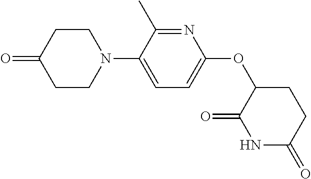 [SMILES: Cc1nc(OC2CCC(=O)NC2=O)ccc1N1CCC(=O)CC1]

The title compound was prepared in a manner similar to that in intermediate 7 step 4. [M+H]⁺=318.2

### Intermediate 88: (7¹R,7³S,E)-1¹,1³,2-trimethyl-5⁶-(4-((methylamino)methyl)piperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

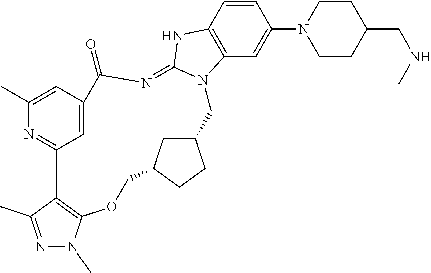 [SMILES: CNCC1CCN(c2ccc3c(c2)N2C[C@@H]4CC[C@H](COc5c(c(C)nn5C)-c5cc(cc(C)n5)C(=O)/N=C/2N3)C4)CC1]

The title compound was prepared in a manner similar to that in intermediate 2 with intermediate 39. [M+H]+=583.5

### Intermediate 89: 3-(3-methyl-4-(4-oxopiperidin-1-yl)phenoxy)piperidine-2,6-dione

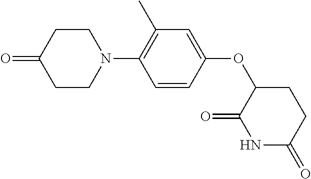 [SMILES: Cc1cc(OC2CCC(=O)NC2=O)ccc1N1CCC(=O)CC1]

The title compound was prepared in a manner similar to that in intermediate 31. [M+H]⁺=317.2

### Intermediate 90: (7¹R,7³S,E)-1¹,1³,2⁶-trimethyl-5⁶-(methyl(piperidin-4-yl)amino)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

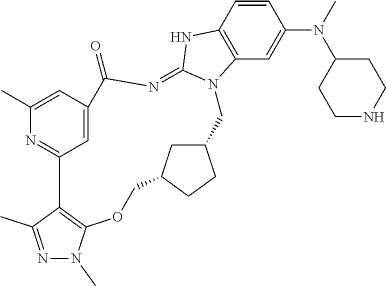 [SMILES: Cc1cc2cc(n1)-c1c(C)nn(C)c1OC[C@H]1CC[C@H](C1)CN1/C(=N/C2=O)Nc2ccc(N(C)C3CCNCC3)cc21]

The title compound was prepared in a manner similar to that in intermediate 2. [M+H]⁺=569.5

### Intermediate 91: 3-(4-((3S,4R)-3-fluoro-4-(piperazin-1-yl)piperidin-1-yl)phenoxy)piperidine-2,6-dione

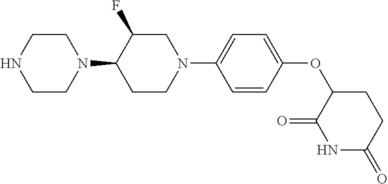 [SMILES: O=C1CCC(Oc2ccc(N3CC[C@@H](N4CCNCC4)[C@@H](F)C3)cc2)C(=O)N1]

The title compound was prepared in a manner similar to that in intermediates 31 and 20. [M+H]⁺=391.3

### Intermediate 93: (7¹S,7³R,E)-5⁶-((R)-3-(methoxymethyl)piperazin-1-yl)-1¹,1³,2⁶-trimethyl-5²,5³-dihydro-1H,5¹H-9-oxa-4-aza-5(2,1)-benzo[d]imidazola-2(2,4)-pyridina-1(4,5)-pyrazola-7(1,3)-cyclopentanacyclononaphan-3-one

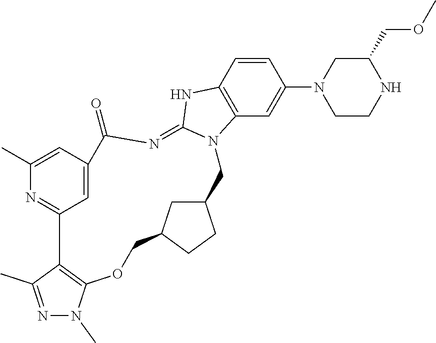

The title compound was prepared in a manner similar to that in intermediates 1 and 2. [M+H]⁺=585.4.

### Intermediate 94: (7¹R,7³S,E)-5⁵-methoxy-5⁶-((S)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

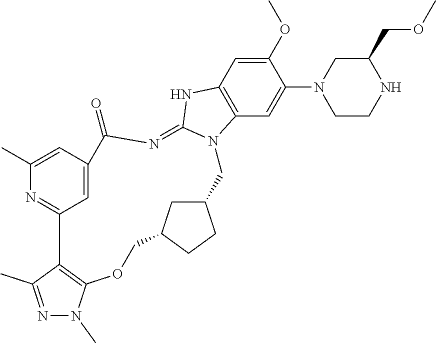

The title compound was prepared in a manner similar to that in intermediates 1 and 2. [M+H]⁺=615.4.

### Intermediate 95: (7¹R,7³S,E)-5-methoxy-1¹,1³,2⁶)-trimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

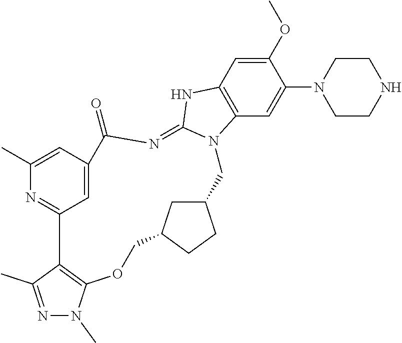 [SMILES: COc1cc2c(cc1N1CCNCC1)N1C[C@@H]3CC[C@H](COc4c(c(C)nn4C)-c4cc(cc(C)n4)C(=O)/N=C/1N2)C3]

The title compound was prepared in a manner similar to that in intermediates 1 and 2. [M+H]⁺=571.5.

### Intermediate 96: (7¹R,7³S,E)-1¹,1³,2⁶-trimethyl-5⁶-(3-(methylamino)azetidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

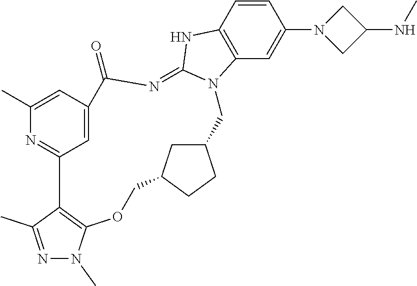

The title compound was prepared in a manner similar to that in intermediate 2. [M+H]⁺=541.5.

### Intermediate 98: (7¹R,7³S,E)-1¹-(2-hydroxyethyl)-5⁶-((R)-3-(methoxymethyl)piperazin-1-yl)-1³,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

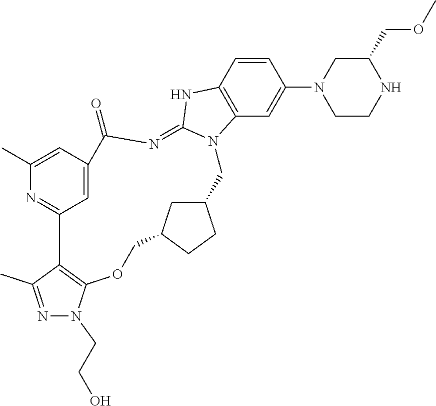

The title compound was prepared in a manner similar to that in intermediates 75 and 76. [M+H]⁺=615.5.

### Intermediate 99: (7¹R,7³S,E)-1¹-(2-hydroxyethyl)-5⁶-((S)-3-(methoxymethyl)piperazin-1-yl)-1³,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

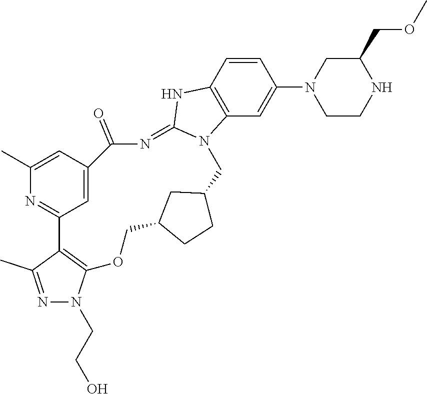

The title compound was prepared in a manner similar to that in intermediates 75 and 76. [M+H]⁺=615.5.

### Intermediate 100: 3-(4-(3-aminoazetidin-1-yl)-3,5-difluorophenoxy)piperidine-2,6-dione

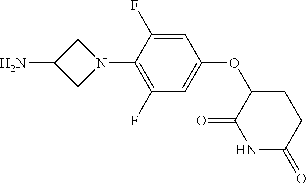 [SMILES: NC1CN(c2c(F)cc(OC3CCC(=O)NC3=O)cc2F)C1]

The title compound was prepared in a manner similar to that in intermediate 31 (steps 1) and 63 (steps 1 and 3). [M+H]⁺=312.5.

### Intermediate 101: (7¹R,7³S,E)-1¹,1³,2⁶-trimethyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

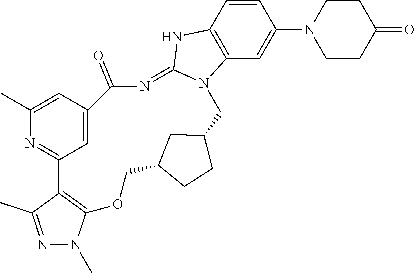 [SMILES: Cc1cc2cc(n1)-c1c(C)nn(C)c1OC[C@H]1CC[C@H](C1)CN1/C(=N/C2=O)Nc2ccc(N3CCC(=O)CC3)cc21]

The title compound was prepared in a manner similar to that in intermediate 64. [M+H]⁺=554.3.

### Intermediate 102: 3-((4,6-dimethyl-5-(4-oxopiperidin-1-yl)pyridin-2-yl)amino)piperidine-2,6-dione

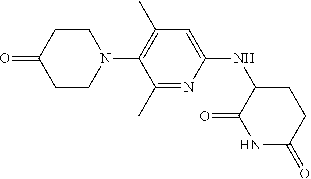 [SMILES: Cc1cc(NC2CCC(=O)NC2=O)nc(C)c1N1CCC(=O)CC1]

The title compound was prepared in a manner similar to that in intermediate 32. [M+H]⁺=331.3.

### Intermediate 103: (7¹R,7³S,E)-1¹-(2-methoxyethyl)-5⁶-((R)-3-(methoxymethyl)piperazin-1-yl)-1³,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

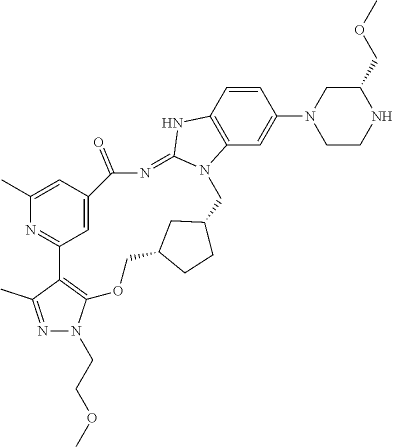

The title compound was prepared in a manner similar to that in intermediates 74, 75 and 2. [M+H]⁺=629.6.

### Intermediate 104: (7¹R,7³S,E)-1¹-(2-methoxyethyl)-1³,2⁶-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

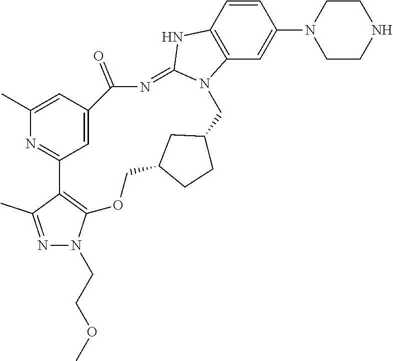 [SMILES: COCCn1nc(C)c2c1OC[C@H]1CC[C@H](C1)CN1/C(=N/C(=O)c3cc(C)nc-2c3)Nc2ccc(N3CCNCC3)cc21]

The title compound was prepared in a manner similar to that in intermediates 74, 75 and 2. [M+H]⁺=585.5.

### Intermediate 105: (7¹R,7³S,E)-1¹,2⁶-dimethyl-5⁶-((R)-3-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

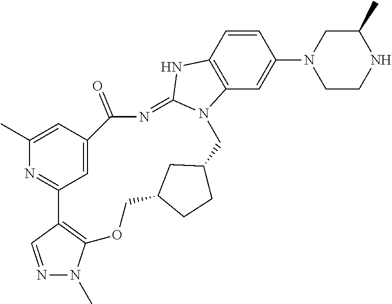

The title compound was prepared in a manner similar to that in intermediate 2. [M+H]⁺=541.5.

### Intermediate 106: 2-((S)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-56-yl)piperazin-2-yl)acetonitrile

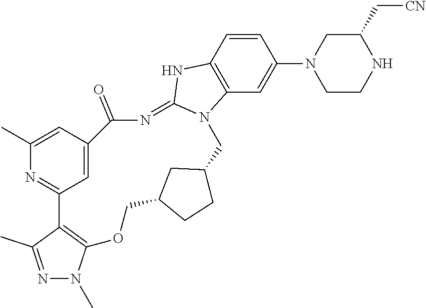 [SMILES: Cc1cc2cc(n1)-c1c(C)nn(C)c1OC[C@H]1CC[C@H](C1)CN1/C(=N/C2=O)Nc2ccc(N3CCN[C@@H](CC#N)C3)cc21]

The title compound was prepared in a manner similar to that in intermediate 2. [M+H]⁺=580.4.

### Intermediate 107: 3-(2,6-difluoro-4-(4-(2-oxopiperazin-1-yl)piperidin-1-yl)phenyl)piperidine-2,6-dione

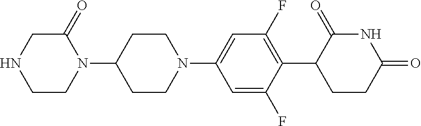 [SMILES: O=C1CCC(c2c(F)cc(N3CCC(N4CCNCC4=O)CC3)cc2F)C(=O)N1]

### Step 1: tert-butyl 4-(1-(4-(2,6-bis(benzyloxy)pyridin-3-yl)-3,5-difluorophenyl)piperidin-4-yl)-3-oxopiperazine-1-carboxylate

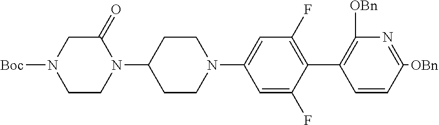 [SMILES: CC(C)(C)OC(=O)N1CCN(C2CCN(c3cc(F)c(-c4ccc(OCc5ccccc5)nc4OCc4ccccc4)c(F)c3)CC2)C(=O)C1]

To a stirred solution of intermediate 6 (1.5 g, 3.1 mmol) and tert-butyl 3-oxo-4-(piperidin-4-yl)piperazine-1-carboxylate (1.2 g, 4.2 mmol) in DMA (30 mL) were added Cs₂CO₃ (3.17 g, 9.75 mmol), Pd₂(dba)₃ (300 mg, 0.325 mmol) and Ruphos (300 mg, 0.65 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (200 mL), washed with water (3×100 mL) and brine (100 mL). The organic layer was dried over anhydrous Na₂SO₄, filtered and concentrated under reduced pressure. The residue was purified by silica gel column chromatography, eluted with PE/EtOAc (4:1) to afford the product (360 mg, 16.9%); [M+H]⁺=685.7.

### Step 2: tert-butyl 4-(1-(4-(2,6-dioxopiperidin-3-yl)-3,5-difluorophenyl)piperidin-4-yl)-3-oxopiperazine-1-carboxylate

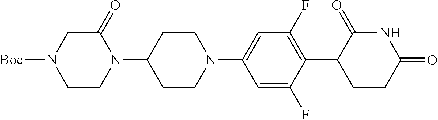 [SMILES: CC(C)(C)OC(=O)N1CCN(C2CCN(c3cc(F)c(C4CCC(=O)NC4=O)c(F)c3)CC2)C(=O)C1]

tert-butyl 4-(1-(4-(2,6-bis(benzyloxy)pyridin-3-yl)-3,5-difluorophenyl)piperidin-4-yl)-3-oxopiperazine-1-carboxylate (360 mg, 0.53 mmol) was dissolved in DMF (8 mL) and iPrOH (4 mL). Pd/C (350 mg, 10 wt. %, wet) was added to the solution in one portion. The resulting mixture was stirred under hydrogen atmosphere (1 atm) at 50° C. overnight. The solid was filtered off and the filtrate was concentrated to give the crude product. The crude was purified by silica gel column chromatography, eluted with DMC/MeOH (20:1) to afford the product (200 mg, 75.1%); [M+H]⁺=507.5.

### Step 3: 3-(2,6-difluoro-4-(4-(2-oxopiperazin-1-yl)piperidin-1-yl)phenyl)piperidine-2,6-dione

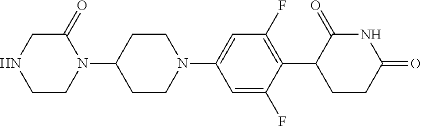 [SMILES: O=C1CCC(c2c(F)cc(N3CCC(N4CCNCC4=O)CC3)cc2F)C(=O)N1]

To a 100-mL round-bottomed flask equipped with a magnetic stir bar was added tert-butyl 4-(1-(4-(2,6-dioxopiperidin-3-yl)-3,5-difluorophenyl)piperidin-4-yl)-3-oxopiperazine-1-carboxylate (200 mg, 0.53 mmol), DCM (6 mL) and TFA (2 mL). After stirring for 1 h at room temperature, the reaction mixture was concentrated under reduced pressure to afford the product as TFA salt (270 mg), which was used without further purification. [M+H]⁺=407.4.

### Intermediate 108: (7¹R,7³S,E)-5⁵-fluoro-5⁶-((R)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

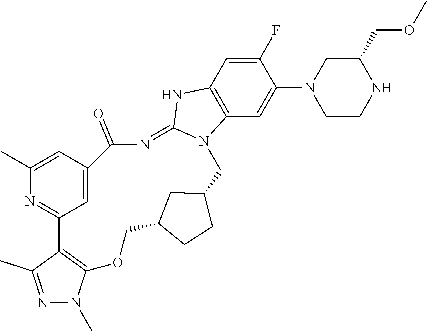

The title compound was prepared in a manner similar to that in intermediates 1 and 2. [M+H]⁺=603.5.

### Intermediate 109: 3-((4,6-dimethyl-5-(4-oxopiperidin-1-yl)pyridin-2-yl)oxy)piperidine-2,6-dione

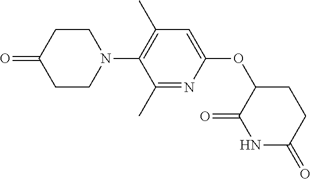 [SMILES: Cc1cc(OC2CCC(=O)NC2=O)nc(C)c1N1CCC(=O)CC1]

The title compound was prepared in a manner similar to that in intermediate 87. [M+H]⁺=332.2.

### Intermediate 110: (7¹R,7³S,E)-1¹,1³,2⁶-trimethyl-5⁶-(4-methyl-4-((methylamino)methyl)piperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

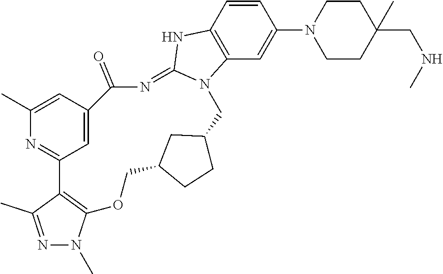 [SMILES: CNCC1(C)CCN(c2ccc3c(c2)N2C[C@@H]4CC[C@H](COc5c(c(C)nn5C)-c5cc(cc(C)n5)C(=O)/N=C/2N3)C4)CC1]

The title compound was prepared in a manner similar to that in intermediates 1 and 2. [M+H]⁺=597.6.

### Intermediate 111: (7¹R,7³S,E)-5⁵-fluoro-5⁶-((S)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

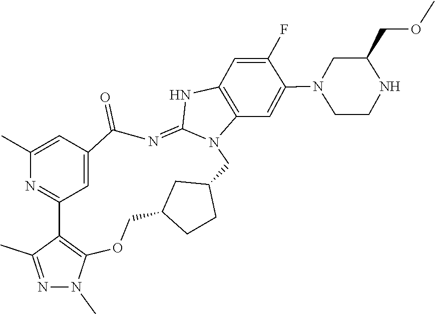

The title compound was prepared in a manner similar to that in intermediates 1 and 2. [M+H]⁺=603.5.

### Intermediate 112: (7¹R,7³S,E)-5⁶-((R)-3-(methoxymethyl)piperazin-1-yl)-1¹,1³,2⁶,5⁵-tetramethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

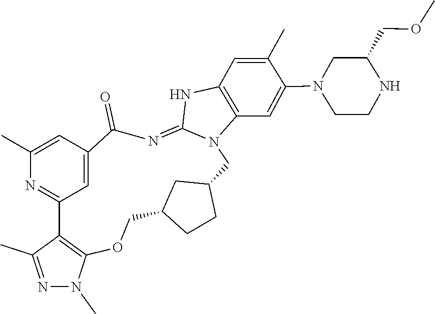

The title compound was prepared in a manner similar to that in intermediates 1 and 2. [M+H]⁺=599.5.

### Intermediate 113: (7¹R,7³S,E)-5⁶-((S)-3-(methoxymethyl)piperazin-1-yl)-1¹,1³,2⁶,5⁵-tetramethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

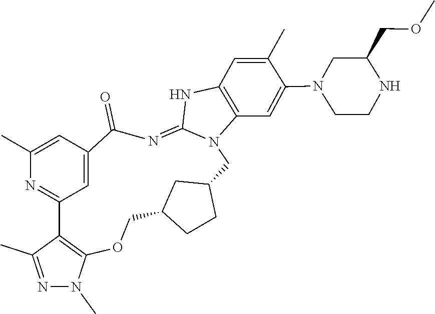

The title compound was prepared in a manner similar to that in intermediates 1 and 2. [M+H]⁺=599.5.

### Intermediate 114: (7¹R,7³S,E)-2⁶-chloro-1¹,1³-dimethyl-5⁶-(piperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

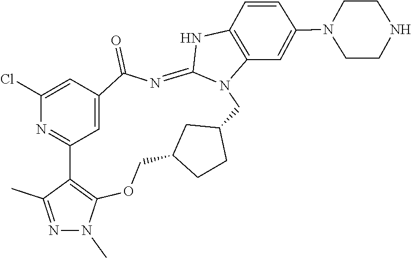 [SMILES: Cc1nn(C)c2c1-c1cc(cc(Cl)n1)C(=O)/N=C1\Nc3ccc(N4CCNCC4)cc3N1C[C@@H]1CC[C@H](CO2)C1]

The title compound was prepared in a manner similar to that in intermediates 1 and 2. [M+H]⁺=561.3.

### Intermediate 115: (R)-2-(1-(4-(2,6-dioxopiperidin-3-yl)-3,5-difluorophenyl)piperidin-4-yl)acetaldehyde

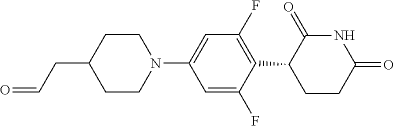 [SMILES: O=CCC1CCN(c2cc(F)c([C@H]3CCC(=O)NC3=O)c(F)c2)CC1]

The title compound was prepared in a manner similar to that in intermediate 44. [M+H]⁺=350.2.

### Intermediate 116: (R)-1-(4-((R)-2,6-dioxopiperidin-3-yl)-3,5-difluorophenyl)pyrrolidine-3-carbaldehyde

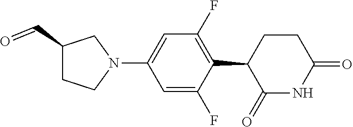 [SMILES: O=C[C@@H]1CCN(c2cc(F)c([C@H]3CCC(=O)NC3=O)c(F)c2)C1]

### Step 1: (R)-(1-(4-(2,6-bis(benzyloxy)pyridin-3-yl)-3,5-difluorophenyl)pyrrolidin-3-yl)methanol

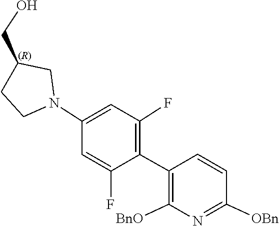

To a solution of (R)-pyrrolidin-3-ylmethanol (9.2 g, 91.0 mmol) in dioxane (600 mL) were added intermediate 6 (52.5 g, 109 mmoL), Pd₂(dba)₃ (8.3 g, 9.0 mmoL), Johnphos (5.39 g, 18.0 mmoL) and K₃PO₄ (57.8 g, 272 mmoL). The resulting mixture was stirred overnight at 110° C. under N₂ atmosphere. After cooled to room temperature and filtered, the filter cake was washed with EA. The filtrate was concentrated under reduced pressure. The crude product was purified by silica column chromatography (EA/PE=0-25%) to afford (R)-(1-(4-(2,6-bis(benzyloxy)pyridin-3-yl)-3,5-difluorophenyl)pyrrolidin-3-yl)methanol (33.5 g, 73.1%). [M+H]+=503.21

### Step 2: (R)-3-(2,6-difluoro-4-((R)-3-(hydroxymethyl)pyrrolidin-1-yl)phenyl)piperidine-2,6-dione

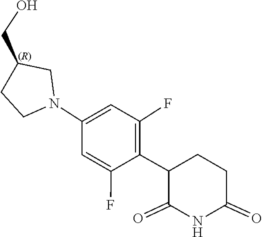

A 1000 mL round bottom flask equipped with a magnetic stirrer, was charged with (R)-(1-(4-(2,6-bis(benzyloxy)pyridin-3-yl)-3,5-difluorophenyl)pyrrolidin-3-yl)methanol (33.5 g, 66.7 mmol), dry THF (400 m1) and Pd/C (10 wt %, 34 g). The resulting mixture was degassed under reduced pressure and purged with H₂ for three times, then stirred overnight at 40° C. The mixture was diluted with THF/EA (1/1), then sonicated in an ultrasonic washer for 10 minutes, followed by filtration through a pad of celite. The filtrate was concentrated under vacuum, The residue was purified by trituration (PE/EA=1/1) to afford 3-(2,6-difluoro-4-((R)-3-(hydroxymethyl)pyrrolidin-1-yl)phenyl)piperidine-2,6-dione (16.5 g, 76.7%).

The title compound was purified by prep-chiral-SFC and analyzed by the following conditions corresponding to peak A @ 0.999 min. [M+H]⁺=325.0
Injection Volume (μL): 1.00
Column Name: (S,S) WHELK-01 4.6*50 mm, 3.5 μm
Solvent A: CO₂
Solvent B: MeOH (0.1% DEA)
Temperature (° C.): 35
Flow (mL/min): 4
Gradient (B %): 10% to 50% in 2.0 min, hold 1.0 min at 50%
Back Pressure (psi): 1500

### Step 3: (R)-1-(4-((R)-2,6-dioxopiperidin-3-yl)-3,5-difluorophenyl)pyrrolidine-3-carbaldehyde

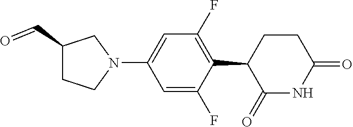 [SMILES: O=C[C@@H]1CCN(c2cc(F)c([C@H]3CCC(=O)NC3=O)c(F)c2)C1]

The title compound was prepared in a manner similar to that in intermediate 33 step 2. [M+H]⁺=323.0.

### Intermediate 117: 3-(2,6-difluoro-4-((S)-3-(piperazin-1-yl)pyrrolidin-1-yl)phenyl)piperidine-2,6-dione

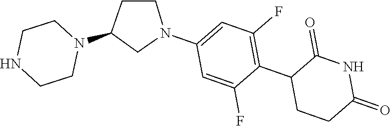 [SMILES: O=C1CCC(c2c(F)cc(N3CC[C@H](N4CCNCC4)C3)cc2F)C(=O)N1]

### Step 1: tert-butyl ((1R,3S)-3-(4-benzylpiperazin-1-yl)cyclopentyl)carbamate

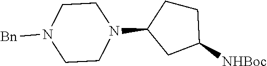 [SMILES: CC(C)(C)OC(=O)N[C@@H]1CC[C@H](N2CCN(Cc3ccccc3)CC2)C1]

A mixture of tert-butyl ((1R,3S)-3-aminocyclopentyl)carbamate (7 g, 35 mmol), benzyl-bis(2-chloroethyl)amine hydrochloride (9.38 g, 35 mmol), KI (581 mg, 3.5 mmol) and K₂CO₃ (24.2 g, 175 mmol) in acetonitrile (140 mL) was stirred at 80° C. for overnight. After the reaction mixture had cooled, it was diluted with dichloromethane and extracted with 1N HCl. The combined organic layers were washed with brine, dried over anhydrous Na₂SO₄, filtered and concentrated under reduced pressure. The residue was purified by reverse phase column (ACN/water (0.05% TFA)=0-30%) to afford tert-butyl ((1R,3S)-3-(4-benzylpiperazin-1-yl)cyclopentyl)carbamate (5.3 g, 42.0%). [M+H]⁺=360.3

### Step 2: (1R,3S)-3-(4-benzylpiperazin-1-yl)cyclopentan-1-amine trifluoroacetate

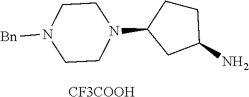 [SMILES: N[C@@H]1CC[C@H](N2CCN(Cc3ccccc3)CC2)C1 ; "CF3COOH"]

To a solution of tert-butyl ((1R,3S)-3-(4-benzylpiperazin-1-yl)cyclopentyl)carbamate (5.3 g, 14.8 mmol) in dichloromethane (40 mL) was added trifluoroacetic acid (20 mL). The reaction mixture was stirred at rt for overnight. The reaction mixture was concentrated under reduced pressure. The residue was purified by trituration (PE/EA=10/1) to afford (1R,3S)-3-(4-benzylpiperazin-1-yl)cyclopentan-1-amine trifluoroacetate (5.4 g, 98.1%). [M+H]⁺=260.11

### Step 3: N-((1R,3S)-3-(4-benzylpiperazin-1-yl)cyclopentyl)-4-(2,6-bis(benzyloxy)pyridin-3-yl)-3,5-difluoroaniline

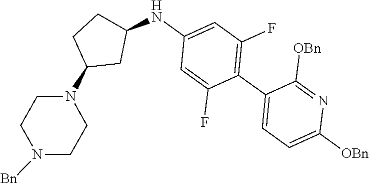 [SMILES: Fc1cc(N[C@@H]2CC[C@H](N3CCN(Cc4ccccc4)CC3)C2)cc(F)c1-c1ccc(OCc2ccccc2)nc1OCc1ccccc1]

A mixture of intermediate 6 (6.0 g, 12.5 mmol), (1R,3S)-3-(4-benzylpiperazin-1-yl)cyclopentan-1-amine trifluoroacetate (5.5 g, 15.0 mmol), Pd₂(dba)₃ (1.1 g, 1.24 mmol), Ruphos (593.7 mg, 1.24 mmol) and Cs₂CO₃ (12.1 g, 37.4 mmol) in dioxane (60 mL) was stirred at 100° C. for overnight under nitrogen atmosphere. The reaction mixture was diluted with H₂O and extracted with EtOAc. The combined organic layers were washed with brine, dried over anhydrous Na₂SO₄, filtered and concentrated under reduced pressure. The residue was purified by silica gel column chromatography (EA/PE=50%-100%) to afford crude solid. Then, the residue was purified by reverse phase column (ACN/water (0.05% TFA)=0-50%) to afford N-((1R,3S)-3-(4-benzylpiperazin-1-yl)cyclopentyl)-4-(2,6-bis(benzyloxy)pyridin-3-yl)-3,5-difluoroaniline (5.3 g, 64.3%). [M+H]⁺=661.33.

### Step 4: tert-butyl 4-((1S,3R)-3-((4-(2,6-dioxopiperidin-3-yl)-3,5-difluorophenyl)amino)cyclopentyl)piperazine-1-carboxylate

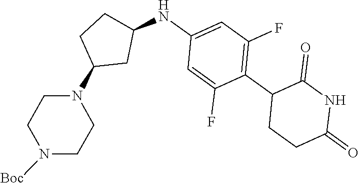 [SMILES: CC(C)(C)OC(=O)N1CCN([C@H]2CC[C@@H](Nc3cc(F)c(C4CCC(=O)NC4=O)c(F)c3)C2)CC1]

A mixture of N-((1R,3S)-3-(4-benzylpiperazin-1-yl)cyclopentyl)-4-(2,6-bis(benzyloxy)pyridin-3-yl)-3,5-difluoroaniline (5.0 g, 7.57 mmol), Boc₂O (2.0 g, 9.08 mmol) and Pd/C (5.0 g) in THF (80 mL) was stirred at 50° C. for overnight under H₂ atmosphere. After the reaction mixture had cooled, it was filtered and the filtrate was concentrated under reduced pressure. The residue was purified by silica gel column chromatography (MeOH/DCM=0-5%) to afford tert-butyl 4-((1S,3R)-3-((4-(2,6-dioxopiperidin-3-yl)-3,5-difluorophenyl)amino)cyclopentyl)piperazine-1-carboxylate (2.64 g, 71.3%). [M+H]⁺=493.25

### Step 5: 3-(2,6-difluoro-4-((S)-3-(piperazin-1-yl)pyrrolidin-1-yl)phenyl)piperidine-2,6-dione

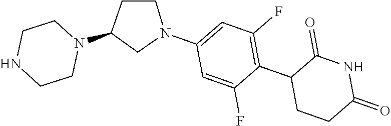 [SMILES: O=C1CCC(c2c(F)cc(N3CC[C@H](N4CCNCC4)C3)cc2F)C(=O)N1]

The title compound was prepared in a manner similar to that in intermediate 2 step 2. [M+H]⁺=379.2.

### Intermediate 118: (7¹R,7³S,E)-5⁶-((R)-3-ethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

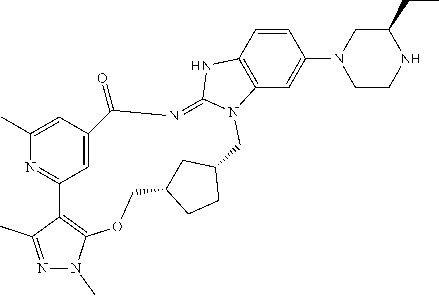 [SMILES: CC[C@@H]1CN(c2ccc3c(c2)N2C[C@@H]4CC[C@H](COc5c(c(C)nn5C)-c5cc(cc(C)n5)C(=O)/N=C/2N3)C4)CCN1]

The title compound was prepared in a manner similar to that in intermediate 1 and 2. [M+H]⁺=569.2.

### Intermediate 119: (7¹R,7³S,E)-5⁶-((S)-3-ethylpiperazin-1-yl)-1%,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

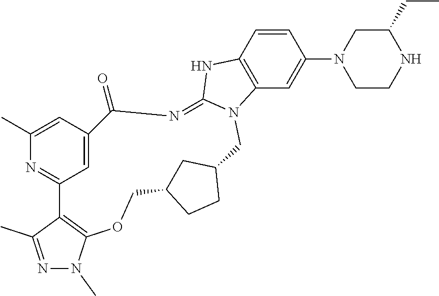 [SMILES: CC[C@H]1CN(c2ccc3c(c2)N2C[C@@H]4CC[C@H](COc5c(c(C)nn5C)-c5cc(cc(C)n5)C(=O)/N=C/2N3)C4)CCN1]

The title compound was prepared in a manner similar to that in intermediate 1 and 2. [M+H]⁺=569.2.

### Intermediate 120: (7¹R,7³S,E)-1¹,1³,2-trimethyl-5⁶-((R)-2-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

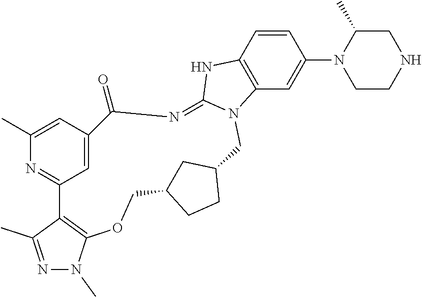 [SMILES: Cc1cc2cc(n1)-c1c(C)nn(C)c1OC[C@H]1CC[C@H](C1)CN1/C(=N/C2=O)Nc2ccc(N3CCNC[C@H]3C)cc21]

The title compound was prepared in a manner similar to that in intermediate 1 and 2. [M+H]⁺=555.2.

### Intermediate 121: (7¹R,7³S,E)-5⁶-((2R,5S)-2,5-dimethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

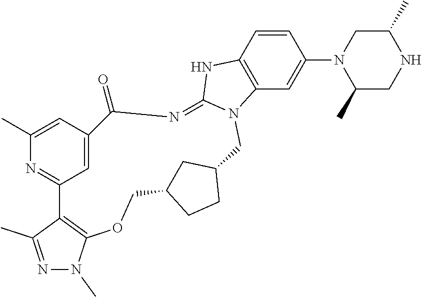

The title compound was prepared in a manner similar to that in intermediate 1 and 2. [M+H]⁺=569.2.

### Intermediate 122: (7¹R,7³S,E)-5⁶-(2,5-diazabicyclo[2.2.2]octan-2-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

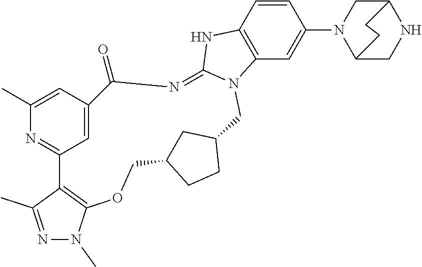 [SMILES: Cc1cc2cc(n1)-c1c(C)nn(C)c1OC[C@H]1CC[C@H](C1)CN1/C(=N/C2=O)Nc2ccc(N3CC4CCC3CN4)cc21]

The title compound was prepared in a manner similar to that in intermediate 1 and 2. [M+H]⁺=567.4

### Intermediate 123: (7¹R,7³S,E)-1¹,1³,2⁶-trimethyl-5⁶-((S)-2-methylpiperazin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

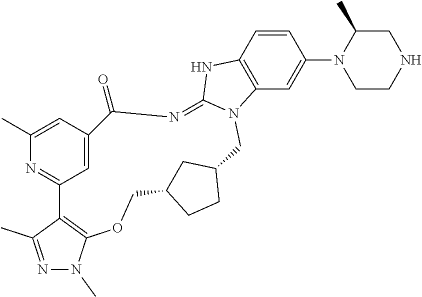 [SMILES: Cc1cc2cc(n1)-c1c(C)nn(C)c1OC[C@H]1CC[C@H](C1)CN1/C(=N/C2=O)Nc2ccc(N3CCNC[C@@H]3C)cc21]

The title compound was prepared in a manner similar to that in intermediate 1 and 2. [M+H]⁺=555.3

### Intermediate 124: (7¹R,7³S,E)-5⁶-((2R,5R)-2,5-dimethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

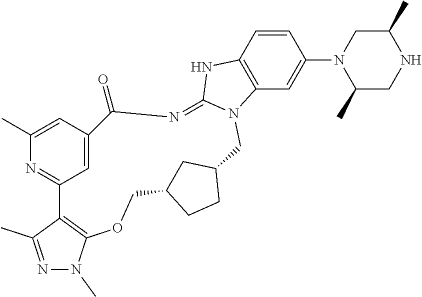

The title compound was prepared in a manner similar to that in intermediate 1 and 2. [M+H]⁺=569.2.

### Intermediate 125: (7¹R,7³S,E)-5⁶-((2S,5S)-2,5-dimethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

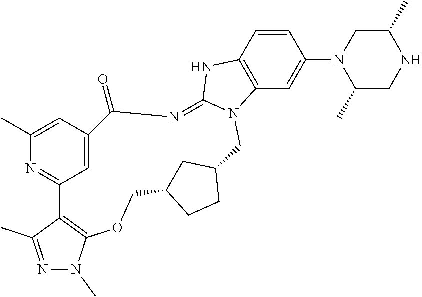

The title compound was prepared in a manner similar to that in intermediate 1 and 2. [M+H]⁺=569.2.

### Intermediate 126: (7¹R,7³S,E)-5⁶-((2S,6S)-2,6-dimethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

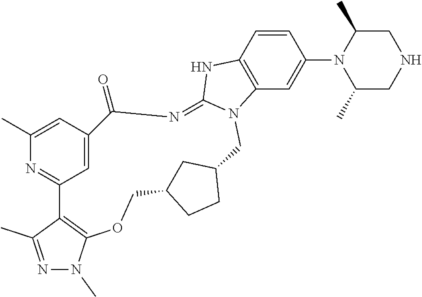 [SMILES: Cc1cc2cc(n1)-c1c(C)nn(C)c1OC[C@H]1CC[C@H](C1)CN1/C(=N/C2=O)Nc2ccc(N3[C@@H](C)CNC[C@@H]3C)cc21]

The title compound was prepared in a manner similar to that in intermediate 1 and 2. [M+H]⁺=569.2.

### Intermediate 127: (7¹R,7³S,E)-5⁶-((2S,5R)-2,5-dimethylpiperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

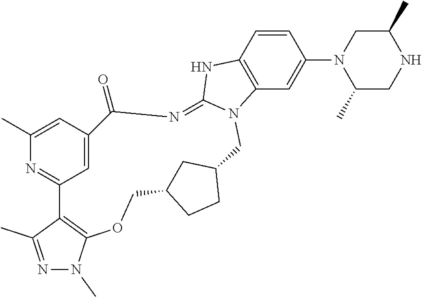

The title compound was prepared in a manner similar to that in intermediate 1 and 2. [M+H]⁺=569.2.

### Intermediate 128: (7¹R,7³S,E)-5⁶-((S)-2-(fluoromethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

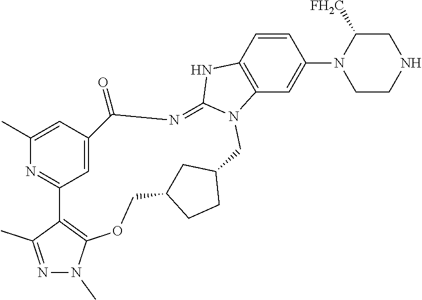

The title compound was prepared in a manner similar to that in intermediate 1 and 2. [M+H]⁺=573.6.

### Intermediate 129: (7¹R,7³S,E)-5⁶-((S)-3-(difluoromethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

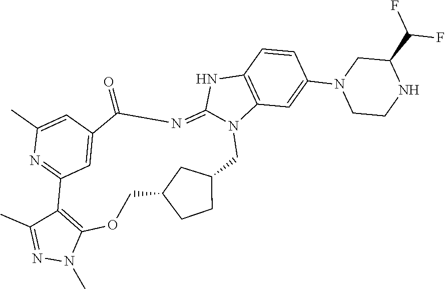 [SMILES: Cc1cc2cc(n1)-c1c(C)nn(C)c1OC[C@H]1CC[C@H](C1)CN1/C(=N/C2=O)Nc2ccc(N3CCN[C@H](C(F)F)C3)cc21]

The title compound was prepared in a manner similar to that in intermediate 1 and 2. [M+H]⁺=591.5.

### Intermediate 130: (7¹R,7³S,E)-5⁶-((R)-4-(azetidin-3-yl)-3-(methoxymethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

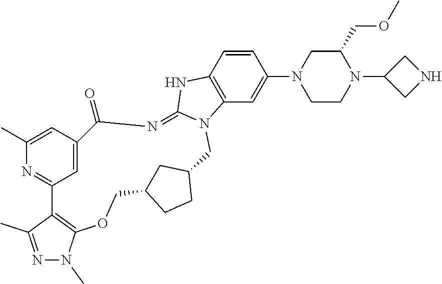 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CNC1]

The title compound was prepared in a manner similar to that in intermediate 61. [M+H]⁺=640.5.

### Intermediate 131: (7¹R,7³S,E)-5⁶-((R)-4-(azetidin-3-yl)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

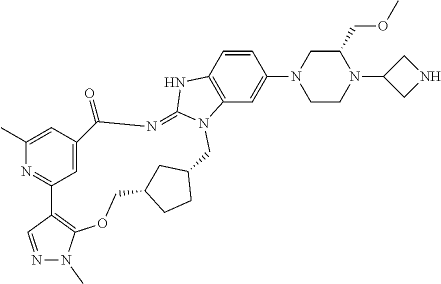 [SMILES: COC[C@H]1CN(c2ccc3c(c2)N2C[C@@H]4CC[C@H](COc5c(cnn5C)-c5cc(cc(C)n5)C(=O)/N=C/2N3)C4)CCN1C1CNC1]

The title compound was prepared in a manner similar to that in intermediate 61. [M+H]⁺=626.6

### Intermediate 132: (7¹R,7³S,E)-5⁶-((R)-3-(fluoromethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

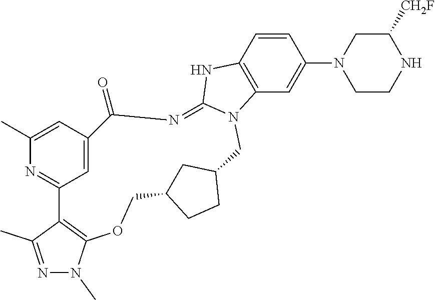

The title compound was prepared in a manner similar to that in intermediate 1 and 2. [M+H]⁺=573.5.

### Intermediate 133: (7¹R,7³S,E)-5⁶-((R)-2-(fluoromethyl)piperazin-1-yl)-1¹,1³,2⁶-trimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

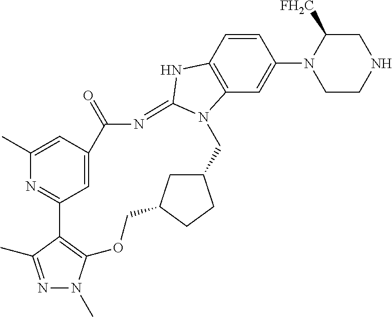

The title compound was prepared in a manner similar to that in intermediate 1 and 2. [M+H]⁺=573.6.

### Intermediate 134: (R)-3-(2,6-difluoro-4-(piperidin-4-ylamino)phenyl)piperidine-2,6-dione

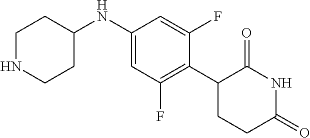 [SMILES: O=C1CCC(c2c(F)cc(NC3CCNCC3)cc2F)C(=O)N1]

The title compound was prepared in a manner similar to that in intermediate 63. [M+H]⁺=324.2.

### Intermediate 135: tert-butyl (S)-4-(2-imino-3-(((1R,3S)-3-(((4-(4-(methoxycarbonyl)-6-methylpyridin-2-yl)-1-methyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate

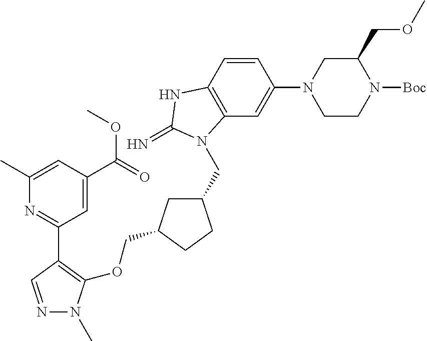

### Step 1: ((1S,3R)-3-(((5-fluoro-2-nitrophenyl)amino)methyl)cyclopentyl)methanol

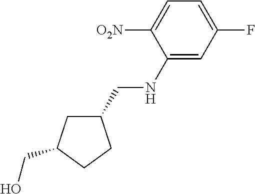 [SMILES: O=[N+]([O-])c1ccc(F)cc1NC[C@@H]1CC[C@H](CO)C1]

2,4-difluoronitrobenzene (1.15 g, 7.26 mmol, 1.1 equiv.) was added to a 50 mL 3-neck round-bottom flask and toluene (10 mL, 10 vol) was added. TEA (1.40 g, 13.9 mmol, 2.1 equiv.) was then added, followed by ((1S,3R)-3-(aminomethyl)cyclopentyl)methanol hydrochloride (1.1 g, 6.6 mmol, 1.0 equiv.). The mixture was heated to 75° C. and stirred at 75° C. overnight. The reaction mixture was cooled to rt and washed with water (˜2×10 vol). The organic layer was collected and solvent was swapped from toluene to acetonitrile. The compound in acetonitrile (5 vol) was directly used for next step. [M+H]⁺=269.2

### Step 2: tert-butyl (S)-4-(3-((((1R,3S)-3-(hydroxymethyl)cyclopentyl)methyl)amino)-4-nitrophenyl)-2-(methoxymethyl)piperazine-1-carboxylate

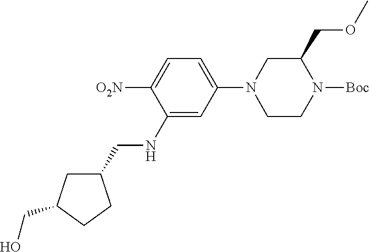 [SMILES: COC[C@@H]1CN(c2ccc([N+](=O)[O-])c(NC[C@@H]3CC[C@H](CO)C3)c2)CCN1C(=O)OC(C)(C)C]

To ((1S,3R)-3-(((5-fluoro-2-nitrophenyl)amino)methyl)cyclopentyl)methanol in acetonitrile was added TEA (1.0 g, 9.9 mmol, 1.5 equiv.) and tert-butyl (S)-2-(methoxymethyl)piperazine-1-carboxylate (1.27 g, 8.0 mmol, 1.2 equiv.) was then added. The mixture was heated to 75° C. and stirred at 75° C. for 24 hours. The reaction mixture was cooled to rt and diluted with DCM (10-15 vol) to dissolve the product. The mixture was washed with water (˜2×10 vol). The organic layer was concentrated and purified by silica column chromatography (EA/DCM=0-30%) to afford tert-butyl (S)-4-(3-((((1R,3S)-3-(hydroxymethyl)cyclopentyl)methyl)amino)-4-nitrophenyl)-2-(methoxymethyl)piperazine-1-carboxylate. [M+H]⁺=479.3

### Step 3: tert-butyl (S)-4-(3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)-4-nitrophenyl)-2-(methoxymethyl)piperazine-1-carboxylate

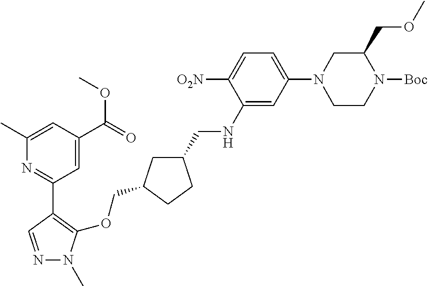

To a 50 mL flask, were added tert-butyl (S)-4-(3-((((1R,3S)-3-(hydroxymethyl)cyclopentyl)methyl)amino)-4-nitrophenyl)-2-(methoxymethyl)piperazine-1-carboxylate (1.14 g, 2.38 mmol, 1.0 equiv.) and methyl 2-(5-hydroxy-1-methyl-1H-pyrazol-4-yl)-6-methylisonicotinate (588 mg, 2.38 mmol, 1.0 equiv.). THF (10 mL, 10 vol) was then added to afford a slurry. Triphenylphosphine (750 mg, 2.86 mmol, 1.2 equiv.) was then added. After that DIAD (578 mg, 2.86 mmol, 1.2 equiv.) was added slowly using a syringe and stirred at R.T for 1 h. The reaction mixture was concentrated and purified by silica column chromatography (EA/DCM=0-30%) to afford tert-butyl (S)-4-(3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)-4-nitrophenyl)-2-(methoxymethyl)piperazine-1-carboxylate (1.55 g, 92.0% yield). [M+H]⁺=708.4

### Step 4: tert-butyl (S)-4-(4-amino-3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)phenyl)-2-(methoxymethyl)piperazine-1-carboxylate

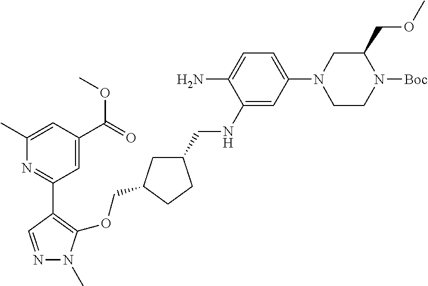

To a stainless steel reactor was charged with tert-butyl (S)-4-(3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)-4-nitrophenyl)-2-(methoxymethyl)piperazine-1-carboxylate (1.55 g, 2.2 mmol, 1.00 eq.) and (5% Wt. Pt, 62.5% H₂O, Kaili Catalyst & New Materials Co., Ltd) Pt/V/C (826 mg, 0.13 mmol, 0.06 eq.). The reactor was evacuated and filled with N₂ three cycles. 25 ml (15 V) anhydrous THF was transferred to the reactor. The reactor was purged with N₂ three times and H₂ three times. The reactor was pressurized with 0.4 MPa of hydrogen and stirred at 40° C. for 24 h. The mixture was filtered, and the solvent was concentrated to afford tert-butyl (S)-4-(4-amino-3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)phenyl)-2-(methoxymethyl)piperazine-1-carboxylate (1.35 g, 90.9% yield). [M+H]⁺=678.4

### Step 5: tert-butyl (S)-4-(2-imino-3-(((1R,3S)-3-(((4-(4-(methoxycarbonyl)-6-methylpyridin-2-yl)-1-methyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate

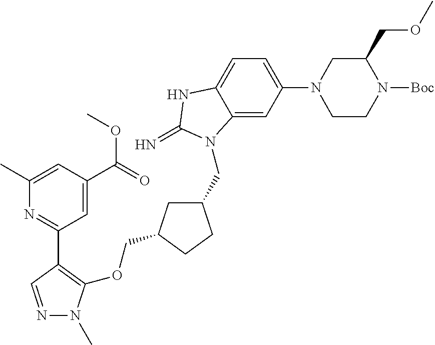

To the solution of tert-butyl (S)-4-(4-amino-3-((((1R,3S)-3-(((4-(4-(methoxycarbonyl)-6-methylpyridin-2-yl)-1-methyl-1H-pyrazol-5-yl)oxy)methyl)cyclopentyl)methyl)amino)phenyl)-2-(methoxymethyl)piperazine-1-carboxylate (1.35 g, 2.0 mmol) and BrCN (0.25 g, 2.4 mmol) in 30 mL MeOH was stirred at room temperature for 2 hrs. The mixture was extracted with DCM, washed with aq. sat. NaHCO₃ and separated. The organic layer was concentrated and purified by silica column chromatography (EA/DCM=0-30%) to afford tert-butyl (S)-4-(2-imino-3-(((1R,3S)-3-(((4-(4-(methoxycarbonyl)-6-methylpyridin-2-yl)-1-methyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate (1.30 g, 92.9% yield). [M+H]⁺=703.4

### Intermediate 136: methyl 2-(5-(((1S,3R)-3-((2-amino-6-bromo-1H-benzo[d]imidazol-1-yl)methyl)cyclopentyl)methoxy)-1-methyl-1H-pyrazol-4-yl)-6-methylisonicotinate

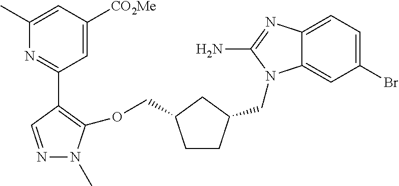 [SMILES: COC(=O)c1cc(C)nc(-c2cnn(C)c2OC[C@H]2CC[C@@H](Cn3c(N)nc4ccc(Br)cc43)C2)c1]

### Step 1: methyl 2-(5-(((1S,3R)-3-((6-bromo-2-((ethoxycarbonyl)amino)-1H-benzo[d]imidazol-1-yl)methyl)cyclopentyl)methoxy)-1-methyl-1H-pyrazol-4-yl)-6-methylisonicotinate

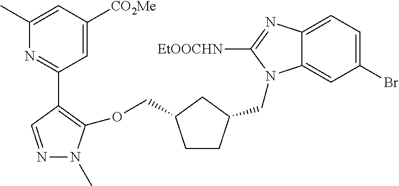

To a solution of methyl 2-(5-(((1S,3R)-3-(((2-amino-5-bromophenyl)amino)methyl)cyclopentyl)methoxy)-1-methyl-1H-pyrazol-4-yl)-6-methylisonicotinate (4.2 g, 8.07 mmol, 1 eq.) (product of intermediate 1 step 8) in THF (40 ml, 10 vol) was added O-ethyl carbonisothiocyanatidate (1.05 eq) in two batches to the reactor, and stirred at 30° C. for 30 minutes after each addition. The reaction was stirred at rt for 1 h to afford an intermediate ([M+H]⁺=659.6). Then, DIPEA (1.56 g, 12.1 mmol, 1.5 eq.) and EDCI (1.86 g, 9.68 mmol, 1.2 eq.) were added. The resulting reaction mixture was stirred at rt for 12 h. The reaction was quenched by H₂O and extracted by DCM. DCM was removed and the crude product was purified by column chromatography (5% MeOH/DCM) to give methyl 2-(5-(((1S,3R)-3-((6-bromo-2-((ethoxycarbonyl)amino)-1H-benzo[d]imidazol-1-yl)methyl)cyclopentyl)methoxy)-1-methyl-1H-pyrazol-4-yl)-6-methylisonicotinate (4.6 g, 93% yield). [M+H]⁺=625.5.

### Step 2: methyl 2-(5-(((1S,3R)-3-((2-amino-6-bromo-1H-benzo[d]imidazol-1-yl)methyl)cyclopentyl)methoxy)-1-methyl-1H-pyrazol-4-yl)-6-methylisonicotinate

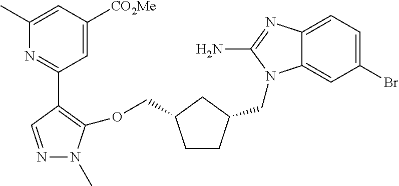 [SMILES: COC(=O)c1cc(C)nc(-c2cnn(C)c2OC[C@H]2CC[C@@H](Cn3c(N)nc4ccc(Br)cc43)C2)c1]

The solution of methyl 2-(5-(((1S,3R)-3-((6-bromo-2-((ethoxycarbonyl)amino)-1H-benzo[d]imidazol-1-yl)methyl)cyclopentyl)methoxy)-1-methyl-1H-pyrazol-4-yl)-6-methylisonicotinate (4.6 g, 7.37 mmol) in CH₃CN (46 ml, 10 vol) and H₂O (4.6 ml, 1 vol) was stirred at 80° C. for 20 h. The mixture was concentrated to afford methyl 2-(5-(((1S,3R)-3-((2-amino-6-bromo-1H-benzo[d]imidazol-1-yl)methyl)cyclopentyl)methoxy)-1-methyl-1H-pyrazol-4-yl)-6-methylisonicotinate (3.9 g, 95.8% yield). [M+H]⁺=553.5.

### Intermediate 137: tert-butyl (R)-4-(2-imino-3-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate

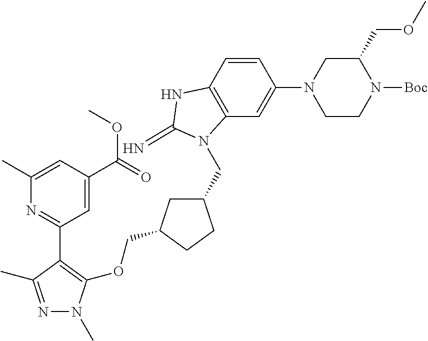

### Step 1: tert-butyl (R)-4-(3-((((1R,3S)-3-(hydroxymethyl)cyclopentyl)methyl)amino)-4-nitrophenyl)-2-(methoxymethyl)piperazine-1-carboxylate

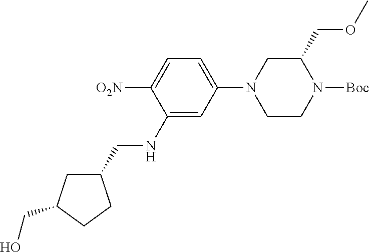 [SMILES: COC[C@H]1CN(c2ccc([N+](=O)[O-])c(NC[C@@H]3CC[C@H](CO)C3)c2)CCN1C(=O)OC(C)(C)C]

To ((1S,3R)-3-(((5-fluoro-2-nitrophenyl)amino)methyl)cyclopentyl)methanol in acetonitrile was added TEA (1.0 g, 9.9 mmol, 1.5 equiv.) and tert-butyl (R)-2-(methoxymethyl)piperazine-1-carboxylate (1.27 g, 8.0 mmol, 1.2 equiv.) was then added. The mixture was heated to 75° C. and stirred at 75° C. for 24 hours. The reaction mixture was cooled to rt and diluted with DCM (10-15 vol) to dissolve the product. The mixture was washed with water (˜2×10 vol). The organic layer was concentrated and purified by silica column chromatography (EA/DCM=0-30%) to afford tert-butyl (R)-4-(3-((((1R,3S)-3-(hydroxymethyl)cyclopentyl)methyl)amino)-4-nitrophenyl)-2-(methoxymethyl)piperazine-1-carboxylate. [M+H]⁺=479.3

### Step 2: tert-butyl (R)-4-(3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)-4-nitrophenyl)-2-(methoxymethyl)piperazine-1-carboxylate

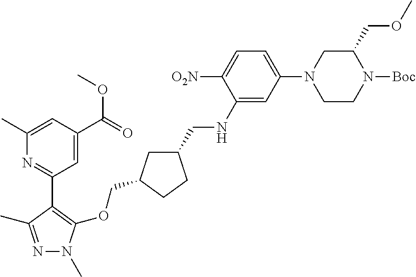

To a 50 mL flask, were added tert-butyl (R)-4-(3-((((1R,3S)-3-(hydroxymethyl)cyclopentyl)methyl)amino)-4-nitrophenyl)-2-(methoxymethyl)piperazine-1-carboxylate (1.14 g, 2.38 mmol, 1.0 equiv.) and methyl 2-(5-hydroxy-1,3-dimethyl-1H-pyrazol-4-yl)-6-methylisonicotinate (588 mg, 2.38 mmol, 1.0 equiv.). THF (10 mL, 10 vol) was then added to afford a slurry. Triphenylphosphine (750 mg, 2.86 mmol, 1.2 equiv.) was then added. After that DIAD (578 mg, 2.86 mmol, 1.2 equiv.) was added slowly using a syringe and stirred at rt for 1 h. The reaction mixture was concentrated and purified by silica column chromatography (EA/DCM=0-30%) to afford tert-butyl (R)-4-(3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)-4-nitrophenyl)-2-(methoxymethyl)piperazine-1-carboxylate (1.55 g, 92.0% yield). [M+H]⁺=722.4

### Step 3: tert-butyl (R)-4-(4-amino-3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)phenyl)-2-(methoxymethyl)piperazine-1-carboxylate

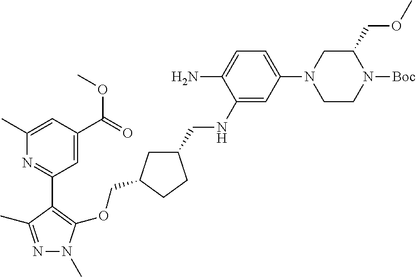

To a stainless steel reactor was charged with tert-butyl (R)-4-(3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)-4-nitrophenyl)-2-(methoxymethyl)piperazine-1-carboxylate (1.55 g, 2.2 mmol, 1.00 eq.) and (5% Wt. Pt, 62.5% H₂O, Kaili Catalyst & New Materials Co., Ltd) Pt/V/C (826 mg, 0.13 mmol, 0.06 eq.). The reactor was evacuated and filled with N₂ three cycles. 25 ml (15 V) anhydrous THF was transferred to the reactor. The reactor was purged with N₂ three times and H₂ three times. The reactor was pressurized with 0.4 MPa of hydrogen and stirred at 40° C. for 24 h. The mixture was filtered, and the solvent was concentrated to afford tert-butyl (R)-4-(4-amino-3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)phenyl)-2-(methoxymethyl)piperazine-1-carboxylate (1.35 g, 90.9% yield). [M+H]⁺=692.4

### Step 4: tert-butyl (R)-4-(2-((ethoxycarbonyl)amino)-1-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-1H-benzo[d]imidazol-6-yl)-2-(methoxymethyl)piperazine-1-carboxylate

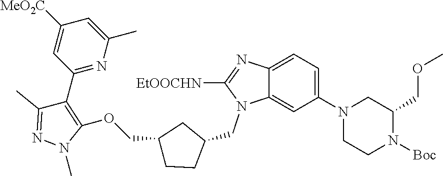

To a solution of tert-butyl (R)-4-(4-amino-3-((((1R,3S)-3-(((4-(4-(methoxycarbonyl)-6-methylpyridin-2-yl)-1,3-dimethyl-1H-pyrazol-5-yl)oxy)methyl)cyclopentyl)methyl)amino)phenyl)-2-(methoxymethyl)piperazine-1-carboxylate (1.35 g, 1.95 mmol, 1 eq.) in THF (15 ml, 10 vol) was added O-ethyl carbonisothiocyanatidate (1.05 eq) in two batches to the reactor, and stirred at 30° C. for 30 minutes after each addition. The reaction was stirred at rt for 1 h to afford an intermediate ([M+H]⁺=823.4). Then, DIPEA (377 mg, 2.9 mmol, 1.5 eq.) and EDCI (450 mg, 2.34 mmol, 1.2 eq.) were added. The resulting reaction mixture was stirred at rt for 12 h. The reaction was quenched by H₂O and extracted by DCM. DCM was removed and the crude product was purified by column chromatography (5% MeOH/DCM) to give tert-butyl (R)-4-(2-((ethoxycarbonyl)amino)-1-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-1H-benzo[d]imidazol-6-yl)-2-(methoxymethyl)piperazine-1-carboxylate (1.38 g, 89% yield). [M+H]⁺=789.4

### Step 5: tert-butyl (R)-4-(2-imino-3-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate

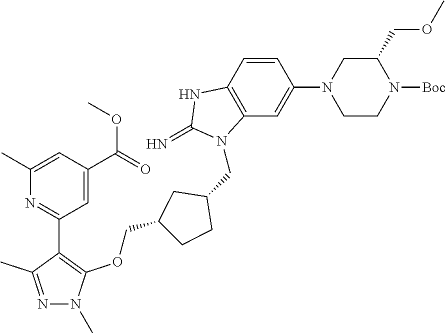

The solution of tert-butyl (R)-4-(2-((ethoxycarbonyl)amino)-1-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-1H-benzo[d]imidazol-6-yl)-2-(methoxymethyl)piperazine-1-carboxylate (1.38 g, 1.75 mmol) in CH₃CN (14 ml, 10 vol) and H₂O (1.5 ml, 1 vol) was stirred at 80° C. for 20 h. The mixture was concentrated to afford tert-butyl (R)-4-(2-imino-3-(((1R,3S)-3-(((4-(4-(methoxycarbonyl)-6-methylpyridin-2-yl)-1,3-dimethyl-1H-pyrazol-5-yl)oxy)methyl)cyclopentyl)methyl)-2,3-dihydro-1H-benzo[d]imidazol-5-yl)-2-(methoxymethyl)piperazine-1-carboxylate (1.15 g, 91.7% yield). [M+H]⁺=717.4

### Intermediate 138: (7¹R,7³S,E)-1³-ethyl-5⁶-((R)-3-(methoxymethyl)piperazin-1-yl)-1¹,2⁶-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

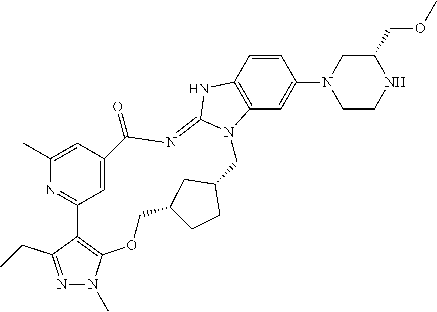 [SMILES: CCc1nn(C)c2c1-c1cc(cc(C)n1)C(=O)/N=C1\Nc3ccc(N4CCN[C@@H](COC)C4)cc3N1C[C@@H]1CC[C@H](CO2)C1]

The title compound was prepared in a manner similar to that in intermediates 1 and 2. [M+H]⁺=599.5.

### Intermediate 139: (7¹R,7³S,E)-2⁶-chloro-5⁶-((R)-3-(methoxymethyl)piperazin-1-yl)-1¹,1³-dimethyl-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

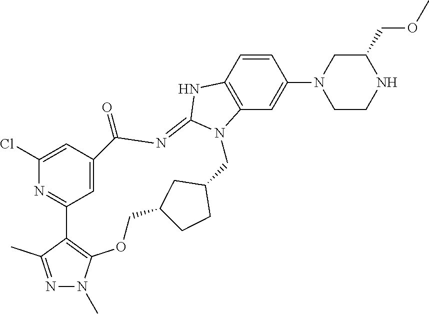

The title compound was prepared in a manner similar to that in intermediates 1 and 2. [M+H]⁺=605.3.

### Intermediate 140: (7¹R,7³S,E)-1¹-(2-hydroxyethyl)-2⁶-methyl-5⁶-(4-oxopiperidin-1-yl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphan-3-one

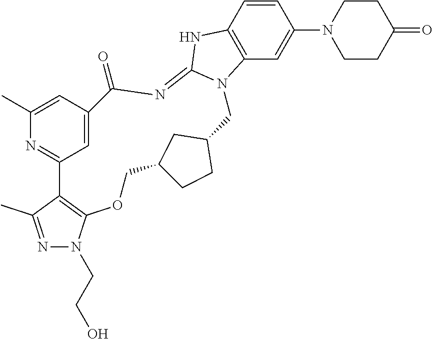 [SMILES: Cc1cc2cc(n1)-c1c(C)nn(CCO)c1OC[C@H]1CC[C@H](C1)CN1/C(=N/C2=O)Nc2ccc(N3CCC(=O)CC3)cc21]

The title compound was prepared in a manner similar to that in intermediates 64 with intermediate 75. [M+H]+=570.3.

### Compound Synthesis

### Example 1: (R)-3-(4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

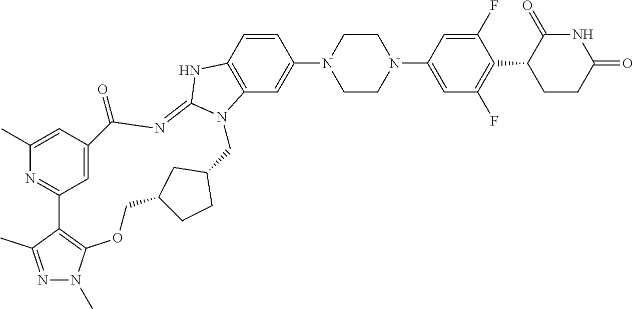 [SMILES: Cc1cc2cc(n1)-c1c(C)nn(C)c1OC[C@H]1CC[C@H](C1)CN1/C(=N/C2=O)Nc2ccc(N3CCN(c4cc(F)c([C@H]5CCC(=O)NC5=O)c(F)c4)CC3)cc21]

To a solution of intermediate 2 (80 mg, 0.15 mmol) and intermediate 7 (68 mg, 0.21 mmol) in DCE (8 mL) was added STAB (95 mg, 0.45 mmol). Then the mixture was stirred at 50° C. overnight. The reaction was quenched with sat. aq. NaHCO₃ and extracted with DCM (3×15 mL). The combined organic phase was washed with brine (1×10 mL), dried over Na₂SO₄, filtered and concentrated in vacuum. The residue was purified by silica gel column (DCM:CH₃OH=10:1), followed by Prep-HPLC chromatography to afford the title product (33 mg, 26.1%). ¹H NMR (500 MHz, DMSO) δ 12.42 (s, 1H), 10.87 (s, 1H), 8.52 (s, 1H), 7.89 (s, 1H), 7.45 (s, 1H), 7.36 (d, J=8.7 Hz, 1H), 7.01 (s, 1H), 6.90 (d, J=8.7 Hz, 1H), 6.66 (s, 1H), 6.63 (s, 1H), 4.30 (s, 1H), 4.19 (s, 1H), 4.05 (dd, J=12.5, 5.0 Hz, 3H), 3.81 (d, J=12.1 Hz, 2H), 3.74 (s, 3H), 3.16 (s, 4H), 2.77 (dd, J=15.1, 7.7 Hz, 3H), 2.68 (s, 4H), 2.65-2.57 (m, 2H), 2.55 (s, 3H), 2.53 (d, J=4.0 Hz, 2H), 2.49-2.40 (m, 2H), 2.09 (qd, J=13.4, 4.9 Hz, 1H), 1.99-1.79 (m, 6H), 1.67 (s, 1H), 1.54-1.43 (m, 2H). [M+H]⁺=833.7

### Example 2: 3-(5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

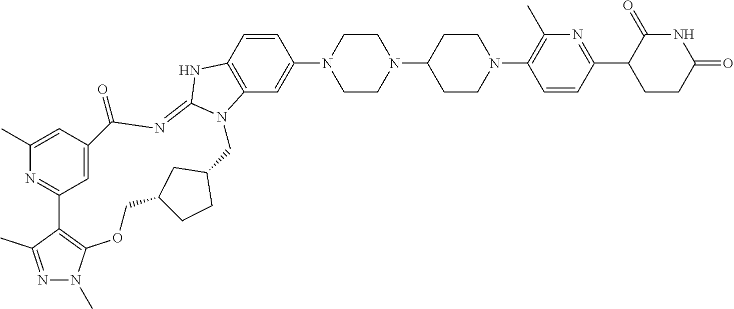 [SMILES: Cc1cc2cc(n1)-c1c(C)nn(C)c1OC[C@H]1CC[C@H](C1)CN1/C(=N/C2=O)Nc2ccc(N3CCN(C4CCN(c5ccc(C6CCC(=O)NC6=O)nc5C)CC4)CC3)cc21]

The title compound was prepared in a manner similar to that in Example 1 with intermediates 2 and 15. ¹H NMR (500 MHz, DMSO) δ 12.56 (s, 1H), 10.88 (s, 1H), 8.64 (s, 1H), 8.02 (s, 1H), 7.60 (s, 1H), 7.56 (d, J=8.1 Hz, 1H), 7.44 (d, J=8.7 Hz, 1H), 7.27 (d, J=7.9 Hz, 1H), 7.17 (s, 1H), 7.01 (d, J=8.3 Hz, 1H), 4.33 (s, 1H), 4.22 (s, 1H), 4.13 (s, 2H), 4.00 (dd, J=9.8, 5.2 Hz, 1H), 3.95 (d, J=11.6 Hz, 2H), 3.77 (s, 3H), 3.72 (d, J=10.9 Hz, 2H), 3.41-3.39 (m, 2H), 3.29 (d, J=10.7 Hz, 4H), 3.08 (d, J=11.8 Hz, 2H), 2.74 (t, J=11.4 Hz, 2H), 2.67-2.58 (m, 7H), 2.48 (s, 3H), 2.25 (d, J=10.1 Hz, 3H), 2.09 (dd, J=13.3, 5.2 Hz, 1H), 1.96-1.80 (m, 6H), 1.68 (s, 1H). [M+H]⁺=812.7

### Example 3: 3-(5-(4-(4-((7¹S,7³R,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

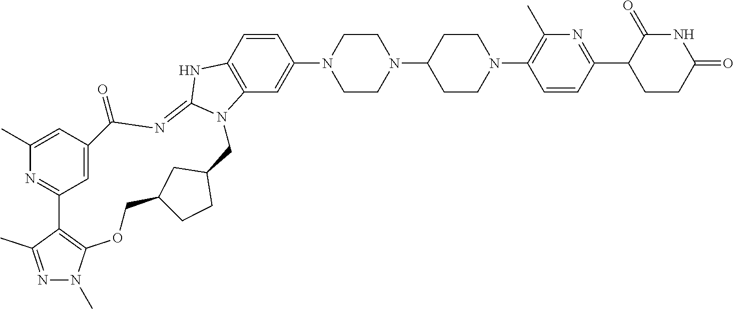 [SMILES: Cc1cc2cc(n1)-c1c(C)nn(C)c1OC[C@@H]1CC[C@@H](C1)CN1/C(=N/C2=O)Nc2ccc(N3CCN(C4CCN(c5ccc(C6CCC(=O)NC6=O)nc5C)CC4)CC3)cc21]

The title compound was prepared in a manner similar to that in Example 1 with intermediates 5 and 15. ¹H NMR (500 MHz, DMSO) δ 12.56 (s, 1H), 10.88 (s, 1H), 8.64 (s, 1H), 8.02 (s, 1H), 7.60 (s, 1H), 7.56 (d, J=8.1 Hz, 1H), 7.44 (d, J=8.7 Hz, 1H), 7.27 (d, J=7.9 Hz, 1H), 7.17 (s, 1H), 7.01 (d, J=8.3 Hz, 1H), 4.33 (s, 1H), 4.22 (s, 1H), 4.13 (s, 2H), 4.00 (dd, J=9.8, 5.2 Hz, 1H), 3.95 (d, J=11.6 Hz, 2H), 3.77 (s, 3H), 3.72 (d, J=10.9 Hz, 2H), 3.41-3.39 (m, 2H), 3.29 (d, J=10.7 Hz, 4H), 3.08 (d, J=11.8 Hz, 2H), 2.74 (t, J=11.4 Hz, 2H), 2.67-2.58 (m, 7H), 2.48 (s, 3H), 2.25 (d, J=10.1 Hz, 3H), 2.09 (dd, J=13.3, 5.2 Hz, 1H), 1.96-1.79 (m, 6H), 1.68 (s, 1H). [M+H]⁺=812.6

### Example 4: 3-(5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-ethylpyridin-2-yl)piperidine-2,6-dione

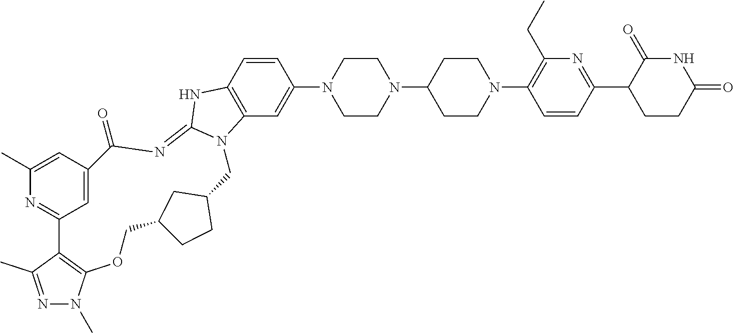 [SMILES: CCc1nc(C2CCC(=O)NC2=O)ccc1N1CCC(N2CCN(c3ccc4c(c3)N3C[C@@H]5CC[C@H](COc6c(c(C)nn6C)-c6cc(cc(C)n6)C(=O)/N=C/3N4)C5)CC2)CC1]

The title compound was prepared in a manner similar to that in Example 1 with intermediates 2 and 17. ¹H NMR (500 MHz, DMSO) δ 12.39 (s, 1H), 10.79 (s, 1H), 8.52 (s, 1H), 7.89 (s, 1H), 7.45 (s, 1H), 7.40 (d, J=8.2 Hz, 1H), 7.37 (d, J=8.7 Hz, 1H), 7.11 (d, J=8.2 Hz, 1H), 7.02 (s, 1H), 6.91 (dd, J=8.8, 1.7 Hz, 1H), 4.30 (s, 1H), 4.19 (t, J=8.0 Hz, 1H), 4.09 (s, 2H), 3.91 (dd, J=7.7, 5.6 Hz, 1H), 3.75 (d, J=5.0 Hz, 3H), 3.18 (s, 4H), 3.07 (s, 2H), 2.80 (td, J=14.8, 7.4 Hz, 1H), 2.75-2.52 (m, 15H), 2.43-2.34 (m, 1H), 2.23-2.09 (m, 2H), 1.99-1.75 (m, 6H), 1.70-1.57 (m, 3H), 1.18 (t, J=7.4 Hz, 3H). [M+H]⁺=826.6.

### Example 5: 3-(4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3,3-dimethyl-2-oxoindolin-1-yl)piperidine-2,6-dione

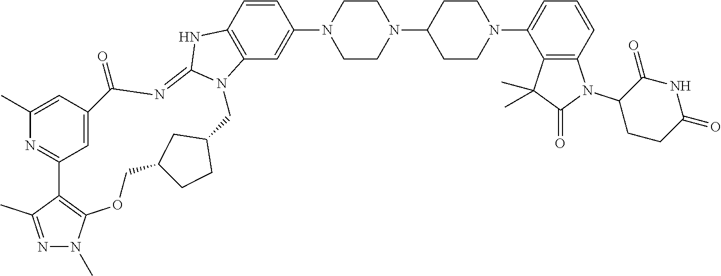 [SMILES: Cc1cc2cc(n1)-c1c(C)nn(C)c1OC[C@H]1CC[C@H](C1)CN1/C(=N/C2=O)Nc2ccc(N3CCN(C4CCN(c5cccc6c5C(C)(C)C(=O)N6C5CCC(=O)NC5=O)CC4)CC3)cc21]

The title compound was prepared in a manner similar to that in Example 1 with intermediates 2 and 10. ¹H NMR (500 MHz, DMSO) δ 12.40 (s, 1H), 11.06 (s, 1H), 8.52 (s, 1H), 7.89 (s, 1H), 7.45 (s, 1H), 7.37 (d, J=8.7 Hz, 1H), 7.23 (t, J=7.9 Hz, 1H), 7.05 (d, J=8.2 Hz, 1H), 7.03 (s, 1H), 6.92 (d, J=9.0 Hz, 1H), 6.80 (s, 1H), 5.31-5.09 (m, 1H), 4.32 (ddd, J=14.6, 11.5, 5.9 Hz, 1H), 4.19 (t, J=7.7 Hz, 1H), 4.14-4.01 (m, 2H), 3.74 (s, 3H), 3.23-3.13 (m, 4H), 2.94-2.86 (m, 3H), 2.85-2.77 (m, 3H), 2.72 (s, 4H), 2.67-2.57 (m, 4H), 2.55 (s, 3H), 2.52 (d, J=1.4 Hz, 1H), 2.41 (t, J=10.7 Hz, 1H), 1.96-1.90 (m, 4H), 1.87-1.79 (m, 2H), 1.68-1.57 (m, 3H), 1.42 (s, 3H), 1.41 (s, 3H). [M+H]⁺=880.7.

### Example 6: 3-(5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-fluoro-6-methylpyridin-2-yl)piperidine-2,6-dione

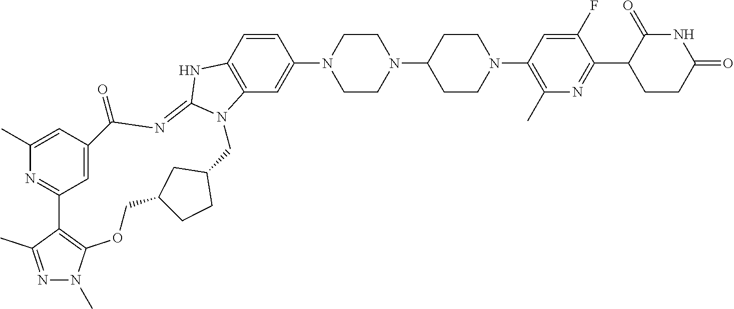 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c(C6CCC(=O)NC6=O)nc5C)CC4)CC3)cc21]

The title compound was prepared in a manner similar to that in Example 1 with intermediates 2 and 12. ¹H NMR (500 MHz, DMSO) δ 12.41 (s, 1H), 10.87 (s, 1H), 8.52 (s, 1H), 7.89 (s, 1H), 7.45 (s, 1H), 7.37 (d, J=8.7 Hz, 1H), 7.32 (d, J=11.6 Hz, 1H), 7.03 (s, 1H), 6.92 (dd, J=8.8, 1.9 Hz, 1H), 4.39-4.25 (m, 1H), 4.23-4.17 (m, 1H), 4.14 (dd, J=11.2, 5.3 Hz, 1H), 4.11-4.05 (m, 1H), 3.74 (s, 3H), 3.23-3.14 (m, 7H), 2.76-2.71 (m, 5H), 2.68 (d, J=5.2 Hz, 1H), 2.66 (s, 1H), 2.64-2.62 (m, 1H), 2.59-2.56 (m, 1H), 2.55 (s, 3H), 2.54-2.52 (m, 2H), 2.44-2.35 (m, 5H), 2.28-2.19 (m, 1H), 2.08-2.01 (m, 1H), 1.94 (d, J=11.3 Hz, 3H), 1.87-1.79 (m, 2H), 1.70-1.58 (m, 3H). [M+H]⁺=830.7.

### Example 7: 3-(5-(2-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)ethyl)-4,6-dimethylpyridin-2-yl)piperidine-2,6-dione

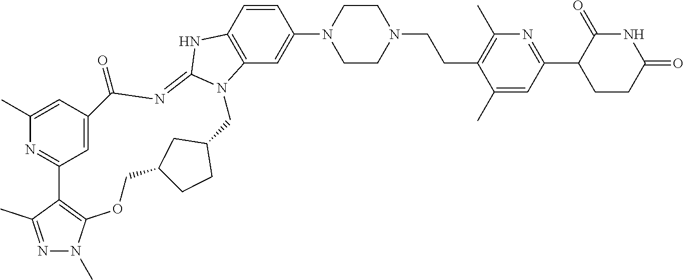 [SMILES: Cc1cc2cc(n1)-c1c(C)nn(C)c1OC[C@H]1CC[C@H](C1)CN1/C(=N/C2=O)Nc2ccc(N3CCN(CCc4c(C)cc(C5CCC(=O)NC5=O)nc4C)CC3)cc21]

To the solution of intermediate 2 (90 mg, 0.17 mmol), intermediate 19 (70 mg, 0.21 mmol) and KI (65 mg, 0.35 mmol) in MeCN (5 mL) and DIEA (67 mg, 0.52 mmol) in DMSO (2 mL) was added. The resulting mixture was heated at 80° C. overnight under N₂. The mixture was quenched with water and extracted with DCM (3×20 mL). The combined organic phase was washed with brine (1×15 mL), dried over Na₂SO₄, filtered and concentrated under reduced pressure to give the crude residue, which was purified by silica column chromatography (DCM:MeOH=100:1-10:1), followed by Prep-HPLC chromatography to afford the title product (21 mg, 16%). ¹H NMR (500 MHz, DMSO) δ 12.42 (s, 1H), 10.79 (s, 1H), 8.52 (s, 1H), 7.89 (s, 1H), 7.45 (s, 1H), 7.37 (d, J=8.7 Hz, 1H), 7.04 (s, 1H), 6.98 (s, 1H), 6.92 (d, J=8.8 Hz, 1H), 4.37-4.26 (m, 1H), 4.19 (t, J=7.9 Hz, 1H), 4.13-4.02 (m, 2H), 3.85 (dd, J=9.3, 5.3 Hz, 1H), 3.74 (s, 3H), 3.26-3.18 (m, 4H), 2.86-2.79 (m, 2H), 2.73-2.66 (m, 4H), 2.64-2.56 (m, 5H), 2.55 (s, 3H), 2.52 (s, 2H), 2.47 (s, 3H), 2.39-2.35 (m, 1H), 2.32 (s, 3H), 2.25-2.16 (m, 1H), 2.12-2.04 (m, 1H), 1.97-1.89 (m, 1H), 1.87-1.79 (m, 2H), 1.71-1.63 (m, 1H). [M+H]⁺=771.6.

### Example 8: 3-(5-(3-((4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)azetidin-1-yl)-4,6-dimethylpyridin-2-yl)piperidine-2,6-dione

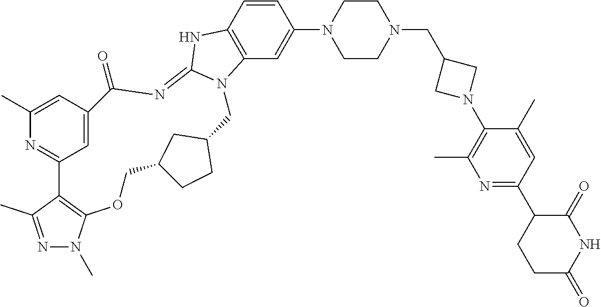 [SMILES: Cc1cc2cc(n1)-c1c(C)nn(C)c1OC[C@H]1CC[C@H](C1)CN1/C(=N/C2=O)Nc2ccc(N3CCN(CC4CN(c5c(C)cc(C6CCC(=O)NC6=O)nc5C)C4)CC3)cc21]

The title compound was prepared in a manner similar to that in Example 7 with intermediates 2 and 18. ¹H NMR (500 MHz, DMSO) δ 10.72 (s, 1H), 8.52 (s, 1H), 7.89 (s, 1H), 7.44 (s, 1H), 7.36 (d, J=8.7 Hz, 1H), 7.03 (d, J=1.7 Hz, 1H), 6.91 (dd, J=8.8, 2.0 Hz, 1H), 6.74 (s, 1H), 4.36-4.28 (m, 1H), 4.25 (t, J=7.4 Hz, 2H), 4.19 (t, J=8.1 Hz, 1H), 4.14-4.00 (m, 2H), 3.78 (t, J=6.5 Hz, 2H), 3.74 (s, 3H), 3.72 (d, J=5.3 Hz, 1H), 3.17 (s, 4H), 2.79 (dt, J=13.7, 6.7 Hz, 1H), 2.68-2.52 (m, 15H), 2.35 (s, 3H), 2.23 (s, 3H), 2.20-2.11 (m, 1H), 2.09-2.01 (m, 1H), 1.96-1.88 (m, 1H), 1.86-1.78 (m, 2H), 1.73-1.62 (m, 1H). [M+H]⁺=812.6.

### Example 9: (R)-3-(4-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

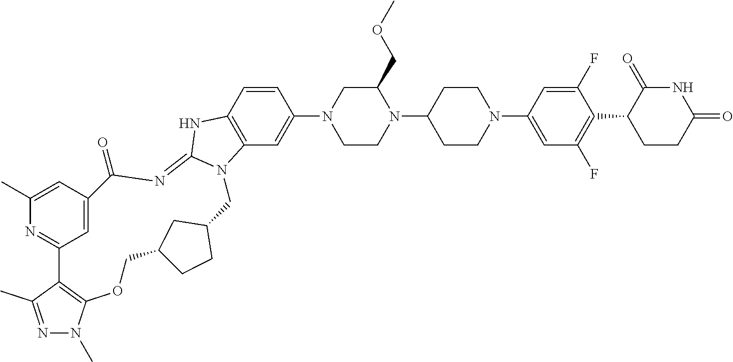 [SMILES: COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in Example 1 with intermediates 3 and 7. H NMR (500 MHz, DMSO) δ 12.41 (s, 1H), 10.87 (s, 1H), 8.52 (s, 1H), 7.89 (s, 1H), 7.45 (s, 1H), 7.37 (d, J=8.7 Hz, 1H), 6.98 (s, 1H), 6.87 (d, J=8.8 Hz, 1H), 6.65 (s, 1H), 6.63 (s, 1H), 4.36-4.25 (m, 1H), 4.19 (t, J=7.9 Hz, 1H), 4.13-4.08 (m, 1H), 4.05 (dd, J=12.6, 5.2 Hz, 2H), 3.83 (d, J=11.9 Hz, 2H), 3.74 (s, 3H), 3.57 (dd, J=9.6, 4.3 Hz, 1H), 3.51 (dd, J=9.7, 5.9 Hz, 1H), 3.30 (s, 3H), 3.28 (d, J=3.0 Hz, 1H), 3.24-3.16 (m, 1H), 3.10-3.01 (m, 2H), 2.98 (dd, J=11.1, 7.1 Hz, 1H), 2.94-2.86 (m, 1H), 2.86-2.68 (m, 7H), 2.67-2.57 (m, 3H), 2.55 (s, 3H), 2.09 (qd, J=12.9, 3.8 Hz, 1H), 2.00-1.81 (m, 5H), 1.74 (d, J=11.9 Hz, 1H), 1.68-1.57 (m, 2H), 1.44 (ddd, J=15.2, 12.4, 3.6 Hz, 1H). [M+H]⁺=877.6.

### Example 10: (R)-3-(4-(4-(5-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-oxa-5,8-diazaspiro[3.5]nonan-8-yl)piperidin-1-yl)-2,6-difluorophenyl)piperidine-2,6-dione

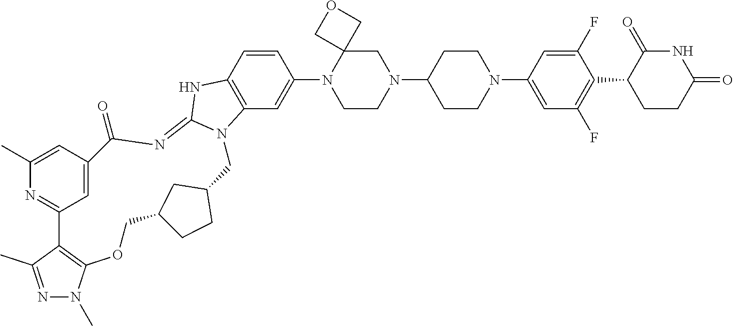 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)CC34COC4)cc21]

The title compound was prepared in a manner similar to that in Example 1 with intermediates 4 and 7. H NMR (500 MHz, DMSO) δ 12.50 (s, 1H), 10.87 (s, 1H), 8.52 (s, 1H), 7.89 (s, 1H), 7.45 (s, 1H), 7.40 (d, J=8.5 Hz, 1H), 7.02 (s, 1H), 6.75 (dd, J=8.5, 1.6 Hz, 1H), 6.66 (s, 1H), 6.63 (s, 1H), 4.53 (d, J=6.1 Hz, 2H), 4.38 (d, J=6.2 Hz, 2H), 4.35-4.27 (m, 1H), 4.19 (t, J=8.2 Hz, 1H), 4.10 (t, J=8.4 Hz, 1H), 4.05 (dd, J=12.6, 5.1 Hz, 2H), 3.80 (d, J=12.5 Hz, 2H), 3.74 (s, 3H), 3.26-3.18 (m, 2H), 2.99 (s, 2H), 2.84-2.74 (m, 3H), 2.65-2.51 (m, 7H), 2.49-2.46 (m, 4H), 2.09 (qd, J=13.0, 3.7 Hz, 1H), 2.00-1.89 (m, 2H), 1.83 (d, J=10.1 Hz, 4H), 1.75-1.66 (m, 1H), 1.56-1.46 (m, 2H). [M+H]⁺=875.7.

### Example 11: 3-(4-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-3,3-dimethyl-2-oxoindolin-1-yl)piperidine-2,6-dione

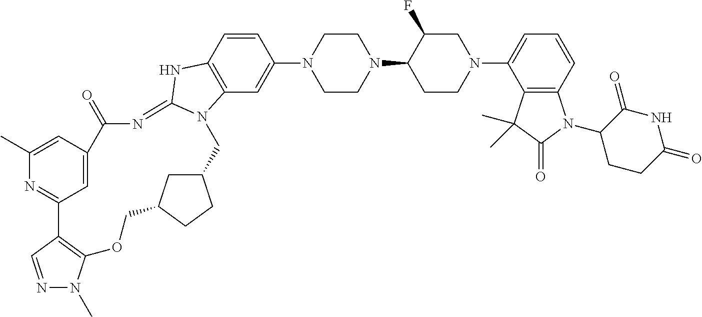 [SMILES: Cc1cc2cc(n1)-c1cnn(C)c1OC[C@H]1CC[C@H](C1)CN1/C(=N/C2=O)Nc2ccc(N3CCN([C@@H]4CCN(c5cccc6c5C(C)(C)C(=O)N6C5CCC(=O)NC5=O)C[C@@H]4F)CC3)cc21]

A mixture of intermediate 1 (104 mg, 0.2 mmol), intermediate 9 (137 mg, 0.3 mmol), Pd₂dba₃ (36.6 mg, 0.04 mmol), Ruphos (36.8 mg, 0.08 mmol) and ^{t-}BuONa (76.8 mg, 0.8 mmol) in DMA (5 mL) was stirred in a round bottom flask at 90° C. for 1 hour under N₂. Water (10 mL) was added, and the mixture was extracted with DCM (20 mL×3). The combined organic layer was dried over Na₂SO₄. The solvent was removed by evaporation, and the residue was purified by silica gel column chromatography to afford the product (40 mg, 22%). ¹H NMR (500 MHz, DMSO) δ 12.43 (s, 1H), 11.07 (s, 1H), 8.53 (s, 1H), 7.89 (s, 1H), 7.45 (s, 1H), 7.38 (d, J=8.7 Hz, 1H), 7.25 (t, J=8.0 Hz, 1H), 7.10-7.00 (m, 2H), 6.95-6.90 (m, 1H), 6.83 (s, 1H), 5.25-5.21 (m, 1H), 5.15-5.06 (m, 1H), 4.38-4.25 (m, 1H), 4.22-4.16 (m, 1H), 4.14-4.02 (m, 2H), 3.74 (s, 3H), 3.24-3.17 (m, 4H), 3.15-2.97 (m, 2H), 2.96-2.80 (m, 7H), 2.64-2.54 (m, 9H), 2.09-1.99 (m, 1H), 1.98-1.76 (m, 6H), 1.69-1.65 (m, 1H), 1.43 (dd, J=8.3, 3.8 Hz, 6H); [M+H]⁺=898.6.

### Example 12: 3-(5-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-6-methylpyridin-2-yl)piperidine-2,6-dione

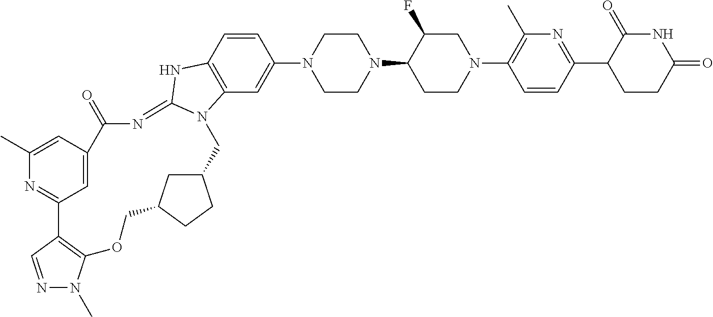 [SMILES: Cc1cc2cc(n1)-c1cnn(C)c1OC[C@H]1CC[C@H](C1)CN1/C(=N/C2=O)Nc2ccc(N3CCN([C@@H]4CCN(c5ccc(C6CCC(=O)NC6=O)nc5C)C[C@@H]4F)CC3)cc21]

The title compound was prepared in a manner similar to that in Example 11 with intermediates 1 and 26. ¹H NMR (500 MHz, DMSO) δ 12.36 (s, 1H), 10.79 (s, 1H), 8.52 (s, 1H), 7.89 (s, 1H), 7.45 (s, 1H), 7.40 (s, 1H), 7.37 (d, J=8.6 Hz, 1H), 7.12 (d, J=8.1 Hz, 1H), 7.04 (s, 1H), 6.93 (d, J=8.8 Hz, 1H), 5.16-5.06 (m, 1H), 4.38-4.26 (m, 1H), 4.23-4.16 (m, 1H), 4.14-4.01 (m, 2H), 3.90 (dd, J=9.5, 5.3 Hz, 1H), 3.74 (s, 3H), 3.19 (d, J=4.4 Hz, 5H), 2.92-2.79 (m, 6H), 2.65-2.63 (m, 1H), 2.62-2.56 (m, 4H), 2.54 (dd, J=6.1, 2.4 Hz, 5H), 2.44-2.42 (m, 3H), 2.26-2.17 (m, 1H), 2.12-2.00 (m, 2H), 1.96-1.75 (m, 5H), 1.72-1.62 (m, 1H). [M+H]⁺=830.6.

### Example 13: 3-(5-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-4,6-dimethylpyridin-2-yl)piperidine-2,6-dione

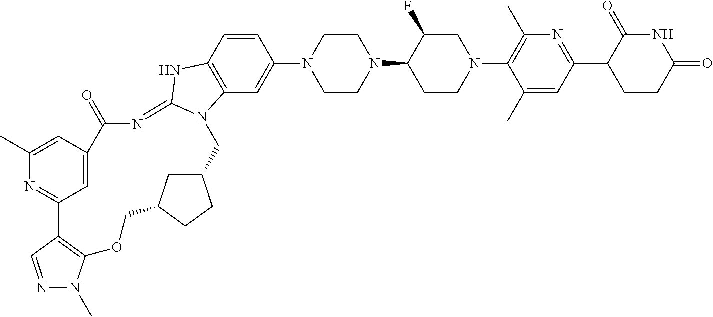 [SMILES: Cc1cc2cc(n1)-c1cnn(C)c1OC[C@H]1CC[C@H](C1)CN1/C(=N/C2=O)Nc2ccc(N3CCN([C@@H]4CCN(c5c(C)cc(C6CCC(=O)NC6=O)nc5C)C[C@@H]4F)CC3)cc21]

The title compound was prepared in a manner similar to that in Example 11 with intermediates 1 and 22. ¹H NMR (500 MHz, DMSO) δ 12.37 (s, 1H), 10.79 (s, 1H), 8.52 (s, 1H), 7.89 (s, 1H), 7.45 (s, 1H), 7.37 (d, J=8.7 Hz, 1H), 7.07-6.90 (m, 3H), 5.07-4.97 (m, 1H), 4.37-4.25 (m, 1H), 4.19 (t, J=7.5 Hz, 1H), 4.14-4.02 (m, 2H), 3.85 (dd, J=8.1, 5.8 Hz, 1H), 3.74 (s, 3H), 3.25-3.07 (m, 6H), 3.01-2.94 (m, 1H), 2.88-2.78 (m, 4H), 2.66-2.53 (m, 10H), 2.47-2.35 (m, 3H), 2.28 (d, J=13.9 Hz, 3H), 2.25-2.14 (m, 1H), 2.12-1.99 (m, 2H), 1.97-1.59 (m, 6H). [M+H]⁺=844.6.

### Example 14: 3-(5-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-3-fluoro-6-methylpyridin-2-yl)piperidine-2,6-dione

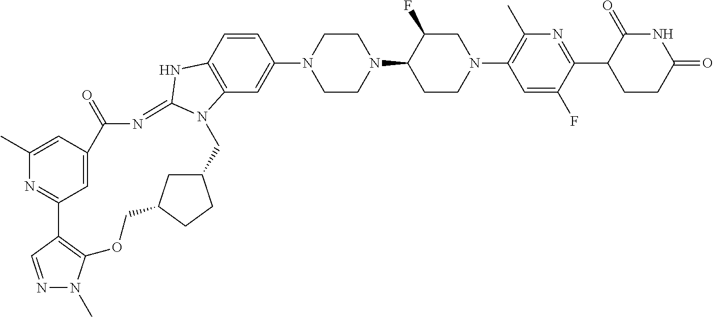 [SMILES: Cc1cc2cc(n1)-c1cnn(C)c1OC[C@H]1CC[C@H](C1)CN1/C(=N/C2=O)Nc2ccc(N3CCN([C@@H]4CCN(c5cc(F)c(C6CCC(=O)NC6=O)nc5C)C[C@@H]4F)CC3)cc21]

The title compound was prepared in a manner similar to that in Example 11 with intermediates 1 and 21. ¹H NMR (500 MHz, DMSO) δ 12.43 (s, 1H), 10.87 (s, 1H), 8.52 (s, 1H), 7.89 (s, 1H), 7.45 (s, 1H), 7.37 (d, J=8.9 Hz, 1H), 7.34 (s, 1H), 7.04 (s, 1H), 6.93 (d, J=9.1 Hz, 1H), 5.16-5.06 (m, 1H), 4.31 (s, 1H), 4.24-4.03 (m, 4H), 3.74 (s, 3H), 3.23-3.15 (m, 4H), 2.94-2.80 (m, 6H), 2.76-2.67 (m, 2H), 2.64-2.57 (m, 5H), 2.54 (d, J=3.8 Hz, 4H), 2.40 (s, 3H), 2.32-2.20 (m, 1H), 2.09-1.99 (m, 2H), 1.96-1.66 (m, 6H). [M+H]+=848.7.

### Example 15: 3-(5-((3R,4S)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-6-methylpyridin-2-yl)piperidine-2,6-dione

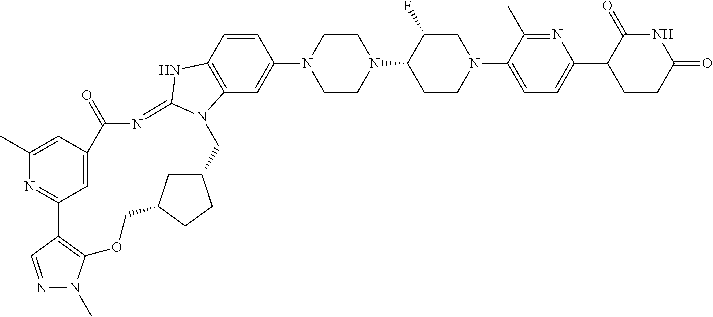 [SMILES: Cc1cc2cc(n1)-c1cnn(C)c1OC[C@H]1CC[C@H](C1)CN1/C(=N/C2=O)Nc2ccc(N3CCN([C@H]4CCN(c5ccc(C6CCC(=O)NC6=O)nc5C)C[C@H]4F)CC3)cc21]

The titled compound was prepared in a manner similar to that in Example 11 with intermediates 1 and 24. ¹H NMR (500 MHz, DMSO) δ 12.42 (s, 1H), 10.79 (s, 1H), 8.53 (s, 1H), 7.89 (s, 1H), 7.45 (s, 1H), 7.38 (t, J=8.2 Hz, 2H), 7.12 (d, J=8.3 Hz, 1H), 7.04 (s, 1H), 6.93 (d, J=9.1 Hz, 1H), 4.31 (s, 1H), 4.19 (s, 1H), 4.10 (s, 1H), 3.90 (dd, J=9.4, 5.4 Hz, 1H), 3.74 (s, 3H), 3.45 (s, 2H), 3.41-3.36 (m, 4H), 3.19 (s, 4H), 2.83 (s, 4H), 2.62-2.61 (m, 1H), 2.59-2.56 (m, 2H), 2.55 (s, 3H), 2.42 (s, 3H), 2.25-2.18 (m, 1H), 2.10-2.03 (m, 2H), 1.96-1.91 (m, 1H), 1.85-1.80 (m, 2H), 1.70-1.65 (m, 1H), 1.24 (s, 3H), 0.88-0.83 (m, 2H); [M+H]+=830.6

### Example 21: 3-(5-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-4-methylpyridin-2-yl)piperidine-2,6-dione

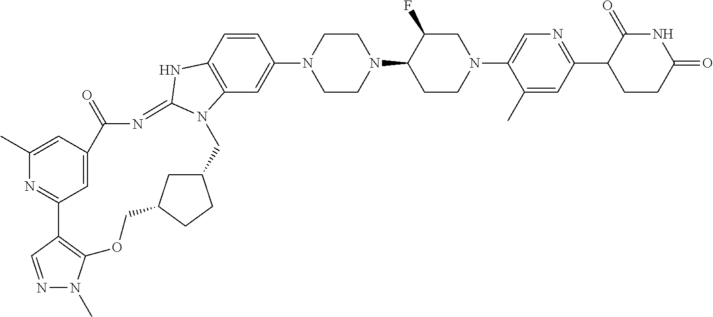 [SMILES: Cc1cc2cc(n1)-c1cnn(C)c1OC[C@H]1CC[C@H](C1)CN1/C(=N/C2=O)Nc2ccc(N3CCN([C@@H]4CCN(c5cnc(C6CCC(=O)NC6=O)cc5C)C[C@@H]4F)CC3)cc21]

The titled compound was prepared in a manner similar to that in Example 11 with intermediates 1 and 27.

¹H NMR (500 MHz, DMSO) δ 12.36 (s, 1H), 10.80 (s, 1H), 8.52 (s, 1H), 8.16 (s, 1H), 7.89 (s, 1H), 7.45 (s, 1H), 7.37 (d, J=8.8 Hz, 1H), 7.15 (s, 1H), 7.04 (s, 1H), 6.93 (d, J=8.7 Hz, 1H), 5.18-5.00 (m, 1H), 4.32 (dd, J=12.3, 10.5 Hz, 1H), 4.19 (t, J=7.1 Hz, 1H), 4.14-4.03 (m, 2H), 3.90 (dd, J=8.8, 5.4 Hz, 1H), 3.74 (s, 3H), 3.29-3.17 (m, 6H), 3.01-2.79 (m, 7H), 2.65-2.56 (m, 6H), 2.55 (s, 3H), 2.28 (s, 3H), 2.23-2.16 (m, 1H), 2.13-2.08 (m, 1H), 2.04-1.98 (m, 1H), 1.95-1.90 (m, 1H), 1.87-1.79 (m, 3H), 1.71-1.61 (m, 1H). [M+H]⁺=830.7.

### Example 23: 3-(5-(4-((4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)piperidin-1-yl)-6-methylpyridin-2-yl)piperidine-2,6-dione

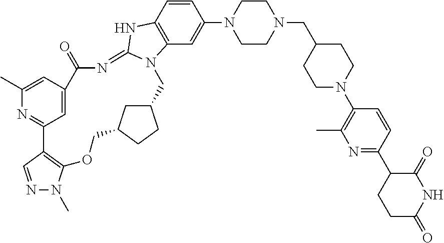 [SMILES: Cc1cc2cc(n1)-c1cnn(C)c1OC[C@H]1CC[C@H](C1)CN1/C(=N/C2=O)Nc2ccc(N3CCN(CC4CCN(c5ccc(C6CCC(=O)NC6=O)nc5C)CC4)CC3)cc21]

The title compound was prepared in a manner similar to that in Example 1 with intermediates 2 and 28.

¹H NMR (500 MHz, DMSO) δ 12.42 (s, 1H), 10.78 (s, 1H), 8.52 (s, 1H), 7.89 (s, 1H), 7.45 (s, 1H), 7.37 (dd, J=8.4, 2.5 Hz, 2H), 7.10 (d, J=8.2 Hz, 1H), 7.03 (s, 1H), 6.92 (d, J=8.9 Hz, 1H), 4.36-4.27 (m, 1H), 4.20 (dd, J=12.3, 8.2 Hz, 1H), 4.11-4.07 (m, 2H), 3.89 (dd, J=9.3, 5.3 Hz, 1H), 3.74 (s, 4H), 3.18 (s, 4H), 3.08 (d, J=11.1 Hz, 3H), 2.60-2.58 (m, 5H), 2.57-2.51 (m, 7H), 2.39 (s, 3H), 2.28 (d, J=7.2 Hz, 2H), 2.23-2.19 (m, 1H), 2.12-2.07 (m, 1H), 1.97-1.78 (m, 6H), 1.74-1.64 (m, 2H), 1.34-1.29 (m, 2H); [M+H]⁺=826.7.

### Example 27: 3-(5-((3R,4S)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-3-fluoro-6-methylpyridin-2-yl)piperidine-2,6-dione

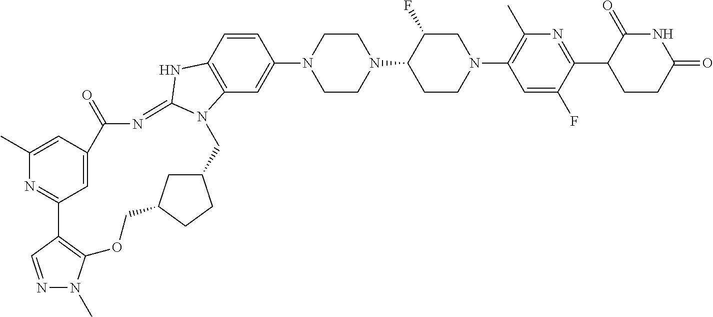 [SMILES: Cc1cc2cc(n1)-c1cnn(C)c1OC[C@H]1CC[C@H](C1)CN1/C(=N/C2=O)Nc2ccc(N3CCN([C@H]4CCN(c5cc(F)c(C6CCC(=O)NC6=O)nc5C)C[C@H]4F)CC3)cc21]

The title compound was prepared in a manner similar to that in Example 11 with intermediates 1 and 20.

¹H NMR (500 MHz, DMSO) δ 12.42 (s, 1H), 10.87 (s, 1H), 8.52 (s, 1H), 7.89 (s, 1H), 7.45 (s, 1H), 7.36 (dd, J=12.3, 10.2 Hz, 2H), 7.04 (s, 1H), 6.93 (d, J=8.8 Hz, 1H), 5.18-5.04 (m, 1H), 4.38-4.01 (m, 5H), 3.74 (s, 3H), 3.43 (s, 1H), 3.26-3.16 (m, 5H), 2.94-2.78 (m, 6H), 2.75-2.54 (m, 9H), 2.40 (s, 3H), 2.26 (dd, J=20.2, 11.4 Hz, 1H), 2.05 (dd, J=11.6, 7.1 Hz, 2H), 1.97-1.62 (m, 6H). [M+H]⁺=848.6.

### Example 35: 3-(5-(3-((4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)azetidin-1-yl)-1-oxoisoindolin-2-yl)piperidine-2,6-dione

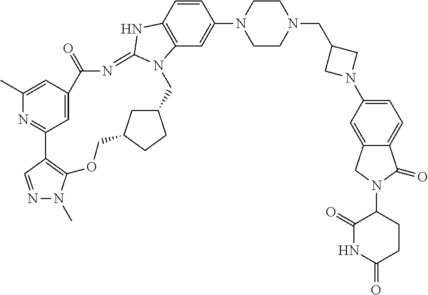 [SMILES: Cc1cc2cc(n1)-c1cnn(C)c1OC[C@H]1CC[C@H](C1)CN1/C(=N/C2=O)Nc2ccc(N3CCN(CC4CN(c5ccc6c(c5)CN(C5CCC(=O)NC5=O)C6=O)C4)CC3)cc21]

The title compound was prepared in a manner similar to that in Example 1 with intermediates 2 and 29.

¹H NMR (500 MHz, DMSO) δ 12.41 (s, 1H), 10.93 (s, 1H), 8.52 (s, 1H), 7.89 (s, 1H), 7.49 (d, J=8.2 Hz, 1H), 7.45 (s, 1H), 7.37 (d, J=8.7 Hz, 1H), 7.03 (s, 1H), 6.92 (d, J=8.6 Hz, 1H), 6.52 (s, 1H), 6.49 (d, J=8.3 Hz, 1H), 5.03-4.99 (m, 1H), 4.35-4.26 (m, 2H), 4.18-4.15 (m, 2H), 4.06-4.02 (m, 4H), 3.74-3.71 (m, 3H), 3.60-3.56 (m, 2H), 3.18-3.15 (m, 4H), 3.06-3.01 (m, 1H), 2.96-2.84 (m, 1H), 2.67-2.64 (m, 2H), 2.62-2.59 (m, 6H), 2.57-2.54 (m, 3H), 2.53-2.49 (m, 3H), 2.35-2.32 (m, 1H), 1.95-1.92 (m, 2H), 1.84-1.79 (m, 2H), 1.71-1.66 (m, 1H); [M+H]⁺=838.7.

### Example 57: 3-(4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenoxy)piperidine-2,6-dione

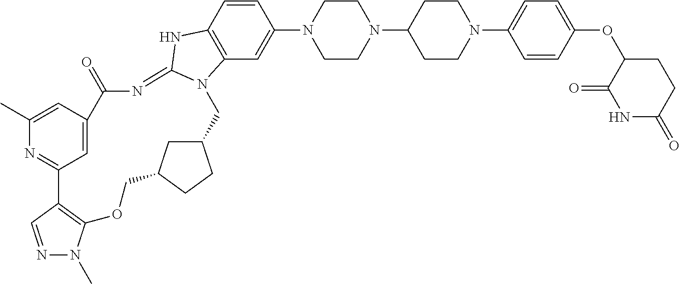 [SMILES: Cc1cc2cc(n1)-c1cnn(C)c1OC[C@H]1CC[C@H](C1)CN1/C(=N/C2=O)Nc2ccc(N3CCN(C4CCN(c5ccc(OC6CCC(=O)NC6=O)cc5)CC4)CC3)cc21]

The title compound was prepared in a manner similar to that in Example 1 with intermediates 2 and 31.

¹H NMR (500 MHz, DMSO) δ 12.42 (s, 1H), 10.88 (s, 1H), 8.52 (s, 1H), 7.89 (s, 1H), 7.45 (s, 1H), 7.36 (d, J=8.7 Hz, 1H), 7.02 (s, 1H), 6.95-6.85 (m, 5H), 5.02-4.49 (m, 1H), 4.30 (s, 1H), 4.18 (d, J=8.0 Hz, 1H), 4.09 (s, 2H), 3.74 (s, 3H), 3.61-3.58 (m, 2H), 3.17 (s, 4H), 2.78-2.63 (m, 6H), 2.62-2.56 (m, 5H), 2.55 (s, 3H), 2.51 (s, 1H), 2.38-2.34 (m, 1H), 2.20-2.14 (m, 1H), 2.13-2.05 (m, 1H), 1.91-1.98 (m, 3H), 1.83 (s, 2H), 1.67 (s, 1H), 1.60-1.53 (m, 2H). [M+H]⁺=813.6.

### Example 59: 3-((4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-fluorophenyl)amino)piperidine-2,6-dione

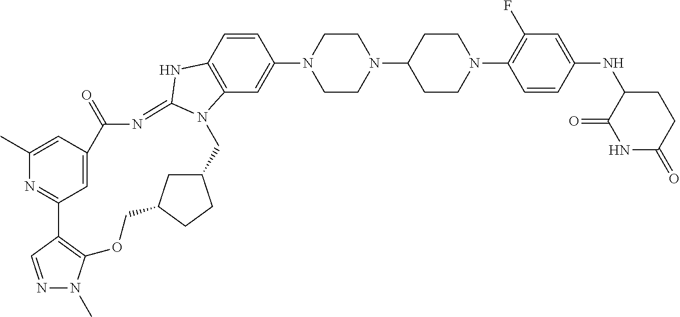 [SMILES: Cc1cc2cc(n1)-c1cnn(C)c1OC[C@H]1CC[C@H](C1)CN1/C(=N/C2=O)Nc2ccc(N3CCN(C4CCN(c5ccc(NC6CCC(=O)NC6=O)cc5F)CC4)CC3)cc21]

To a solution of intermediate 2 (60 mg, 0.11 mmol) and intermediate 32 (50 mg, 0.16 mmol) in DCE (6 mL) was added STAB (72 mg, 0.33 mmol). Then the mixture was stirred at 50° C. overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=10:1), followed by Prep-HPLC chromatography to afford the title product (31 mg, 33.1%). ¹H NMR (500 MHz, DMSO) δ 12.41 (s, 1H), 10.78 (s, 1H), 8.52 (s, 1H), 7.89 (s, 1H), 7.45 (s, 1H), 7.36 (s, 1H), 7.02 (s, 1H), 6.91 (dd, J=8.9, 1.9 Hz, 1H), 6.84 (t, J=9.4 Hz, 1H), 6.51 (dd, J=15.0, 2.4 Hz, 1H), 6.42 (dd, J=8.7, 2.1 Hz, 1H), 5.79 (d, J=7.6 Hz, 1H), 4.35-4.16 (m, 3H), 4.10 (s, 2H), 3.74 (s, 3H), 3.23-3.14 (m, 6H), 2.76-2.66 (m, 5H), 2.65-2.52 (m, 9H), 2.39-2.32 (m, 1H), 2.12-2.05 (m, 1H), 1.95-1.75 (m, 7H), 1.73-1.54 (m, 3H). [M+H]⁺=830.6.

### Example 63: 3-(5-(3-(((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)methyl)azetidin-1-yl)-1-oxoisoindolin-2-yl)piperidine-2,6-dione

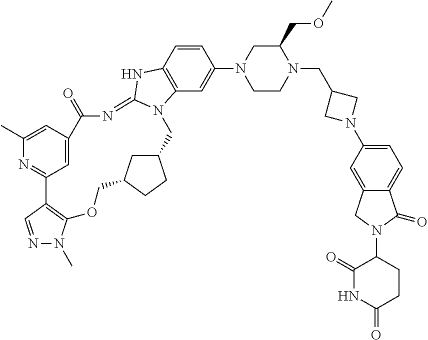 [SMILES: COC[C@@H]1CN(c2ccc3c(c2)N2C[C@@H]4CC[C@H](COc5c(cnn5C)-c5cc(cc(C)n5)C(=O)/N=C/2N3)C4)CCN1CC1CN(c2ccc3c(c2)CN(C2CCC(=O)NC2=O)C3=O)C1]

The title compound was prepared in a manner similar to that in Example 1 with intermediates 3 and 29.

¹H NMR (500 MHz, DMSO) δ 12.43 (s, 1H), 10.93 (s, 1H), 8.52 (s, 1H), 7.89 (s, 1H), 7.49 (d, J=8.3 Hz, 1H), 7.45 (s, 1H), 7.38 (d, J=8.7 Hz, 1H), 7.01 (s, 1H), 6.89 (dd, J=8.8, 1.6 Hz, 1H), 6.52 (s, 1H), 6.48 (dd, J=8.4, 1.6 Hz, 1H), 5.04 (dd, J=13.3, 5.1 Hz, 1H), 4.35-4.25 (m, 2H), 4.21-4.14 (m, 2H), 4.12-3.97 (m, 4H), 3.74 (s, 3H), 3.63-3.54 (m, 3H), 3.51-3.44 (m, 1H), 3.43-3.37 (m, 1H), 3.32 (s, 5H), 3.11-2.81 (m, 7H), 2.73-2.69 (m, 1H), 2.65-2.58 (m, 3H), 2.57 (s, 3H), 2.46-2.41 (m, 1H), 2.40-2.30 (m, 2H), 1.98-1.88 (m, 2H), 1.87-1.80 (m, 2H), 1.72-1.61 (m, 1H); [M+H]⁺=882.7.

### Example 64: (R)-3-(4-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

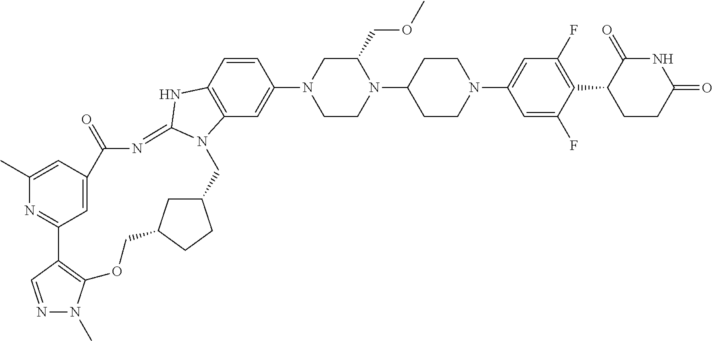 [SMILES: COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1]

To a solution of intermediate 30 (85 mg, 0.15 mmol) and intermediate 7 (67 mg, 0.21 mmol) in DCE (6 mL) was added STAB (96 mg, 0.45 mmol). Then the mixture was stirred at 50° C. overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=10:1), followed by Prep-HPLC chromatography to afford the title product (30 mg, 23.0%).

¹H NMR (500 MHz, DMSO) δ 12.42 (s, 1H), 10.87 (s, 1H), 8.52 (s, 1H), 7.89 (s, 1H), 7.45 (s, 1H), 7.37 (d, J=8.7 Hz, 1H), 6.97 (s, 1H), 6.87 (d, J=8.7 Hz, 1H), 6.64 (d, J=12.8 Hz, 2H), 4.35-4.25 (m, 1H), 4.23-4.15 (m, 1H), 4.11-3.96 (m, 3H), 3.82 (d, J=12.4 Hz, 2H), 3.74 (s, 3H), 3.61-3.55 (m, 1H), 3.53-3.48 (m, 1H), 3.32 (s, 5H), 3.27 (d, J=9.6 Hz, 1H), 3.23-3.16 (m, 1H), 3.09-2.96 (m, 3H), 2.90 (t, J=9.9 Hz, 1H), 2.85-2.70 (m, 5H), 2.69-2.64 (m, 1H), 2.59 (d, J=6.9 Hz, 1H), 2.55 (s, 3H), 2.16-2.03 (m, 1H), 2.00-1.79 (m, 6H), 1.75-1.71 (m, 1H), 1.69-1.55 (m, 2H), 1.50-1.39 (m, 1H); [M+H]⁺=877.7.

### Example 65: 3-(5-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-6-methylpyridin-2-yl)piperidine-2,6-dione

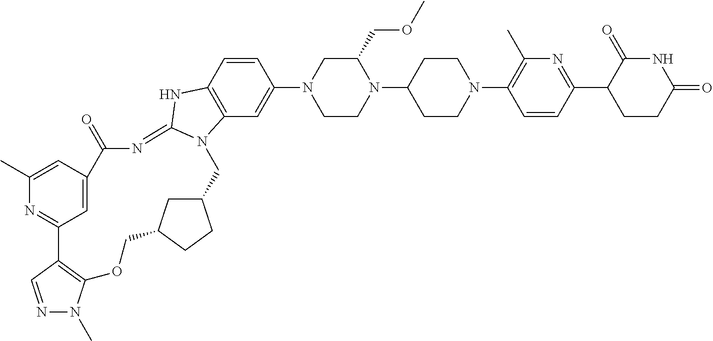 [SMILES: COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2ccc(C3CCC(=O)NC3=O)nc2C)CC1]

The title compound was prepared in a manner similar to that in Example 1 with intermediates 30 and 15.

¹H NMR (500 MHz, DMSO) δ 12.35 (s, 1H), 10.78 (s, 1H), 8.52 (s, 1H), 7.89 (s, 1H), 7.45 (s, 1H), 7.38 (dd, J=8.4, 3.8 Hz, 2H), 7.11 (d, J=8.1 Hz, 1H), 6.99 (s, 1H), 6.89 (d, J=9.7 Hz, 1H), 4.37-4.25 (m, 1H), 4.22-4.18 (m, 1H), 4.11-4.07 (m, 2H), 3.91-3.87 (m, 1H), 3.74 (s, 3H), 3.61-3.55 (m, 1H), 3.52-3.49 (m, 1H), 3.28-3.22 (m, 1H), 3.19-3.11 (m, 2H), 3.09-3.05 (m, 2H), 3.01-2.97 (m, 1H), 2.94-2.80 (m, 2H), 2.72-2.69 (m, 2H), 2.65-2.51 (m, 14H), 2.41 (s, 3H), 2.25-2.15 (m, 1H), 2.13-2.04 (m, 1H), 1.99-1.89 (m, 2H), 1.86-1.73 (m, 4H), 1.73-1.64 (m, 1H), 1.63-1.53 (m, 1H); [M+H]⁺=856.7.

### Example 69: 5-(3-(((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)methyl)azetidin-1-yl)-2-(2,6-dioxopiperidin-3-yl)isoindoline-1,3-dione

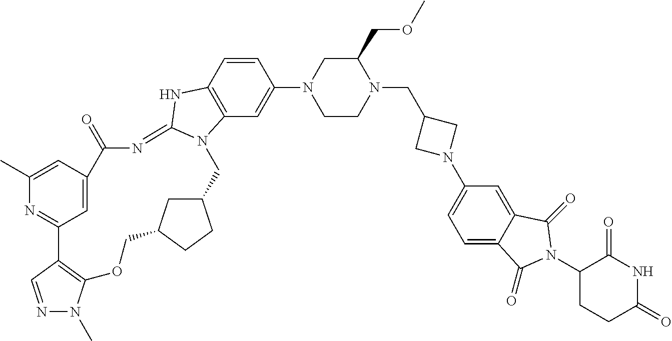 [SMILES: COC[C@@H]1CN(c2ccc3c(c2)N2C[C@@H]4CC[C@H](COc5c(cnn5C)-c5cc(cc(C)n5)C(=O)/N=C/2N3)C4)CCN1CC1CN(c2ccc3c(c2)C(=O)N(C2CCC(=O)NC2=O)C3=O)C1]

To a solution of intermediate 3 (85 mg, 0.15 mmol) and intermediate 33 (72 mg, 0.21 mmol) in DCE (6 mL) was added STAB (96 mg, 0.45 mmol). Then the mixture was stirred at room temperature overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=10:1), followed by Prep-HPLC chromatography to afford the title product (60 mg, 45%).

¹H NMR (500 MHz, DMSO) δ 12.55 (s, 1H), 11.08 (s, 1H), 8.61 (s, 1H), 7.99 (s, 1H), 7.70 (d, J=8.3 Hz, 1H), 7.57 (s, 1H), 7.43 (d, J=8.7 Hz, 1H), 7.13 (d, J=17.3 Hz, 1H), 7.00 (t, J=7.5 Hz, 1H), 6.84 (d, J=1.6 Hz, 1H), 6.71 (dd, J=8.4, 1.9 Hz, 1H), 5.07 (dd, J=12.7, 5.4 Hz, 1H), 4.37-4.18 (m, 5H), 4.15-4.04 (m, 3H), 3.88-3.81 (m, 10H), 3.76-3.72 (m, 3H), 3.69-3.62 (m, 2H), 3.58-3.51 (m, 1H), 3.48-3.42 (m, 1H), 3.32-3.21 (m, 2H), 3.09-2.99 (m, 2H), 2.94-2.84 (m, 1H), 2.64-2.55 (m, 6H), 2.06-1.99 (m, 1H), 1.93-1.88 (m, 1H), 1.84-1.79 (m, 2H), 1.68-1.59 (m, 1H); [M+H]⁺=896.7.

### Example 70: 3-(5-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-3-fluoro-6-methylpyridin-2-yl)piperidine-2,6-dione

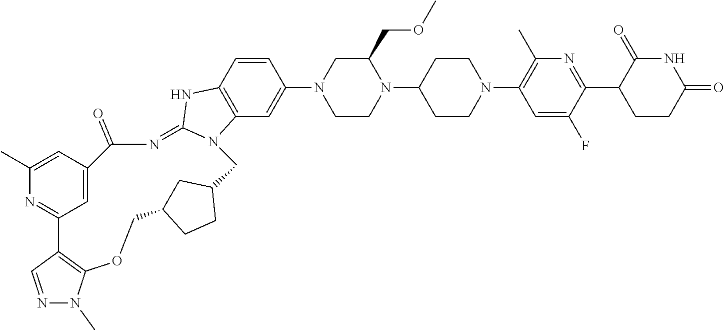

The title compound was prepared in a manner similar to that in Example 1 with intermediates 3 and 12.

¹H NMR (500 MHz, DMSO) δ 12.43 (s, 1H), 10.86 (s, 1H), 8.52 (s, 1H), 7.89 (s, 1H), 7.45 (s, 1H), 7.37 (d, J=8.6 Hz, 1H), 7.33 (d, J=11.6 Hz, 1H), 6.99 (s, 1H), 6.89 (d, J=8.4 Hz, 1H), 4.36-4.26 (m, 1H), 4.23-4.19 (m, 1H), 4.16-4.04 (m, 3H), 3.74 (s, 3H), 3.60-3.55 (m, 1H), 3.53-3.49 (m, 1H), 3.27-3.24 (m, 1H), 3.21-3.16 (m, 2H), 3.09-3.03 (m, 2H), 3.01-2.96 (m, 1H), 2.92-2.82 (m, 3H), 2.76-2.67 (m, 6H), 2.65-2.60 (m, 5H), 2.55 (s, 4H), 2.38 (s, 3H), 2.29-2.20 (m, 1H), 2.06-1.99 (m, 1H)), 1.98-1.91 (m, 2H), 1.87-1.72 (m, 5H), 1.63-1.59 (m, 1H); [M+H]⁺=874.7.

### Example 71: 3-(5-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-3-fluoro-6-methylpyridin-2-yl)piperidine-2,6-dione

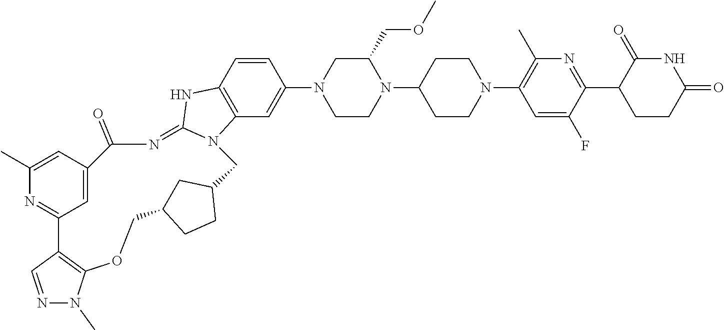

The title compound was prepared in a manner similar to that in Example 1 with intermediate 30 and 12.

¹H NMR (500 MHz, DMSO) δ 12.43 (s, 1H), 10.86 (s, 1H), 8.52 (s, 1H), 7.89 (s, 1H), 7.45 (s, 1H), 7.37 (d, J=8.6 Hz, 1H), 7.33 (d, J=11.6 Hz, 1H), 6.99 (s, 1H), 6.89 (d, J=8.4 Hz, 1H), 4.36-4.26 (m, 1H), 4.23-4.19 (m, 1H), 4.16-4.05 (m, 3H), 3.74 (s, 3H), 3.60-3.55 (m, 1H), 3.53-3.49 (m, 1H), 3.27-3.24 (m, 1H), 3.21-3.16 (m, 2H), 3.11-3.03 (m, 2H), 3.01-2.96 (m, 1H), 2.92-2.82 (m, 3H), 2.76-2.67 (m, 6H), 2.65-2.61 (m, 5H), 2.55 (s, 4H), 2.38 (s, 3H), 2.29-2.20 (m, 1H), 2.06-1.99 (m, 1H)), 1.98-1.91 (m, 2H), 1.88-1.72 (m, 5H), 1.63-1.59 (m, 1H); [M+H]⁺=874.7.

### Example 72: 3-(6-methyl-5-(4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)pyridin-2-yl)piperidine-2,6-dione

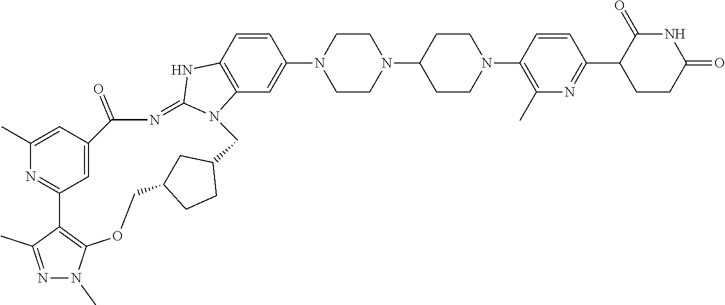

The title compound was prepared in a manner similar to that in Example 1 with intermediates 34 and 15.

¹H NMR (500 MHz, DMSO) δ 12.42 (s, 1H), 10.78 (s, 1H), 8.54 (s, 1H), 7.45 (s, 1H), 7.37 (d, J=8.9 Hz, 2H), 7.11 (d, J=8.2 Hz, 1H), 7.03 (s, 1H), 6.95-6.89 (m, 1H), 4.35-3.98 (m, 4H), 3.89 (dd, J=9.4, 5.3 Hz, 1H), 3.67 (s, 3H), 3.22-3.11 (m, 7H), 2.76-2.70 (m, 4H), 2.68-2.53 (m, 10H), 2.48 (s, 2H), 2.42-2.35 (m, 4H), 2.25-2.17 (m, 1H), 2.08 (dd, J=12.8, 6.0 Hz, 1H), 1.97-1.76 (m, 6H), 1.69-1.57 (m, 3H). [M+H]⁺=826.6.

### Example 73: 3-(5-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-6-methylpyridin-2-yl)piperidine-2,6-dione

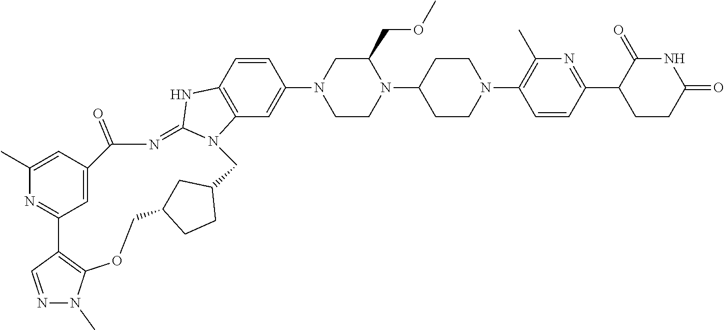

The title compound was prepared in a manner similar to that in Example 1 with intermediates 3 and 15.

¹H NMR (500 MHz, DMSO) δ 12.43 (s, 1H), 10.78 (s, 1H), 8.53 (s, 1H), 7.89 (s, 1H), 7.45 (s, 1H), 7.39 (d, J=3.1 Hz, 1H), 7.37 (d, J=3.6 Hz, 1H), 7.11 (d, J=8.1 Hz, 1H), 6.99 (s, 1H), 6.89 (d, J=8.7 Hz, 1H), 4.39-4.25 (m, 1H), 4.19 (t, J=8.0 Hz, 1H), 4.15-4.00 (m, 2H), 3.89 (dd, J=9.4, 5.3 Hz, 1H), 3.74 (s, 3H), 3.61-3.54 (m, 1H), 3.54-3.47 (m, 1H), 3.36-3.33 (m, 1H), 3.31-3.28 (m, 4H), 3.28-3.22 (m, 1H), 3.18-3.10 (m, 2H), 3.10-3.02 (m, 2H), 3.01-2.94 (m, 1H), 2.93-2.80 (m, 2H), 2.74-2.54 (m, 10H), 2.41 (s, 3H), 2.26-2.17 (m, 1H), 2.12-2.04 (m, 1H), 1.99-1.53 (m, 9H). [M+H]⁺=856.7.

### Example 74: (R)-3-(2,6-difluoro-4-(4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

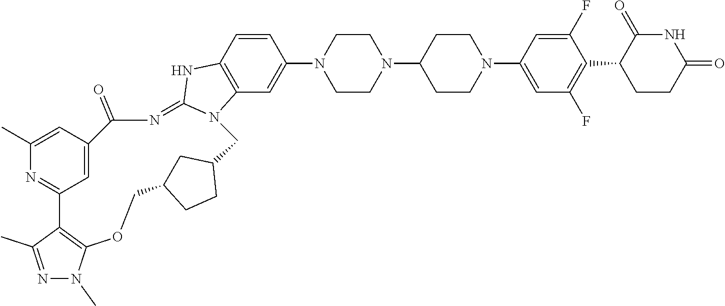

To a solution of intermediate 34 (80 mg, 0.15 mmol) and intermediate 7 (67 mg, 0.21 mmol) in DCE (6 mL) was added STAB (96 mg, 0.45 mmol). Then the mixture was stirred at 50° C. overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=10:1), followed by Prep-HPLC chromatography to afford the title product (35 mg, 28.0%). ¹H NMR (500 MHz, DMSO) δ 12.42 (s, 1H), 10.87 (s, 1H), 8.54 (s, 1H), 7.45 (s, 1H), 7.36 (d, J=8.7 Hz, 1H), 7.02 (s, 1H), 6.90 (dd, J=8.9, 1.9 Hz, 1H), 6.64 (d, J=12.7 Hz, 2H), 4.36-3.97 (m, 5H), 3.81 (d, J=12.6 Hz, 2H), 3.67 (s, 3H), 3.19-3.13 (m, 4H), 2.83-2.63 (m, 9H), 2.58-2.54 (m, 4H), 2.48-2.42 (m, 5H), 2.09 (dt, J=12.8, 9.5 Hz, 1H), 2.00-1.93 (m, 1H), 1.91-1.61 (m, 7H), 1.55-1.44 (m, 2H). [M+H]⁺=847.6.

### Example 75: (R)-3-(4-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(hydroxymethyl)piperazin-1-yl)piperidin-1-yl)-2,6-difluorophenyl)piperidine-2,6-dione

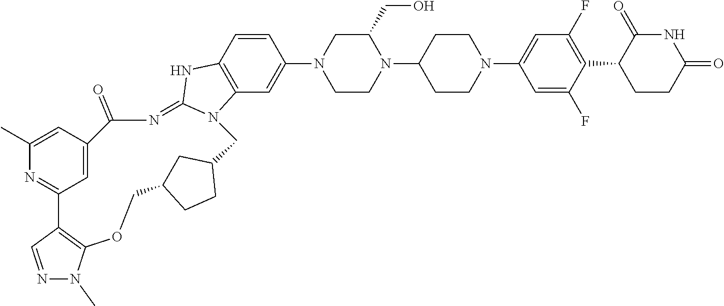

To a solution of intermediate 35 (83 mg, 0.15 mmol) and intermediate 7 (67 mg, 0.21 mmol) in DCE (6 mL) was added STAB (96 mg, 0.45 mmol). Then the mixture was stirred at 50° C. overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=10:1), followed by Prep-HPLC chromatography to afford the title product (10 mg, 7.7%).

¹H NMR (500 MHz, DMSO) δ 12.42 (s, 1H), 10.87 (s, 1H), 8.52 (s, 1H), 7.89 (s, 1H), 7.45 (s, 1H), 7.37 (d, J=8.7 Hz, 1H), 6.97 (s, 1H), 6.89 (d, J=8.7 Hz, 1H), 6.64 (d, J=12.8 Hz, 2H), 4.67-4.58 (m, 1H), 4.33-4.28 (m, 1H), 4.24-4.16 (m, 1H), 4.11-4.06 (m, 3H), 3.88-3.80 (m, 2H), 3.74 (s, 3H), 3.71-3.66 (m, 1H), 3.64-3.59 (m, 1H), 3.07-2.95 (m, 3H), 2.90-2.71 (m, 7H), 2.67-2.62 (m, 3H), 2.59-2.51 (m, 5H), 2.13-2.03 (m, 1H), 2.00-1.89 (m, 3H), 1.85-1.78 (m, 2H), 1.77-1.71 (m, 1H), 1.68-1.57 (m, 2H), 1.52-1.41 (m, 1H); [M+H]⁺=863.7.

### Example 76: 3-(5-((3S,4S)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-6-methylpyridin-2-yl)piperidine-2,6-dione

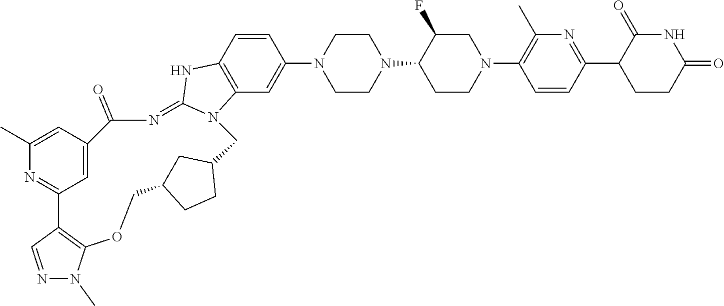

The title compound was prepared in a manner similar to that in Example 11 with intermediates 1 and 36.

¹H NMR (500 MHz, DMSO) δ 12.33 (s, 1H), 10.79 (s, 1H), 8.54 (s, 1H), 7.89 (s, 1H), 7.45 (s, 1H), 7.38 (dd, J=8.4, 4.6 Hz, 2H), 7.12 (d, J=8.1 Hz, 1H), 7.04 (s, 1H), 6.92 (d, J=8.7 Hz, 1H), 5.18-5.03 (m, 1H), 4.40-3.99 (m, 4H), 3.90 (dd, J=9.5, 5.3 Hz, 1H), 3.67 (s, 3H), 3.24-3.14 (m, 6H), 2.92-2.75 (m, 7H), 2.65-2.53 (m, 9H), 2.48 (s, 2H), 2.27-2.17 (m, 1H), 2.13-1.98 (m, 2H), 1.90-1.55 (m, 6H). [M+H]⁺=830.5.

### Example 77: (R)-3-(4-(4-((S)-4-((7¹R,7³S,E)-1¹,2⁶-dimethyl-3-oxo-1³-(trifluoromethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl) piperazin-1-yl)piperidin-1-yl)-2,6-difluorophenyl)piperidine-2,6-dione

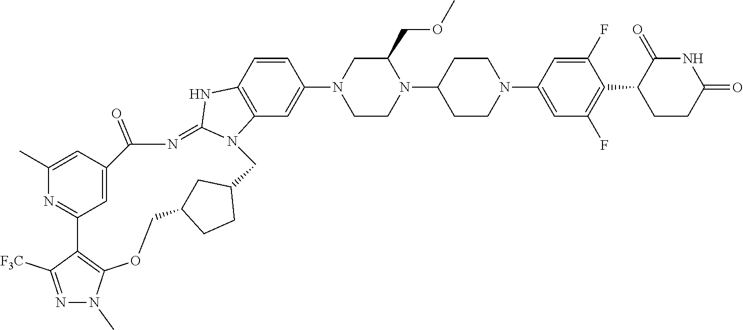

The title compound was prepared in a manner similar to that in Example 1 with intermediates 37 and 7.

¹H NMR (500 MHz, DMSO) δ 12.45 (s, 1H), 10.87 (s, 1H), 8.61 (s, 1H), 7.59 (s, 1H), 7.37 (d, J=8.7 Hz, 1H), 6.99 (s, 1H), 6.88 (d, J=7.8 Hz, 1H), 6.64 (d, J=12.7 Hz, 2H), 4.41-4.24 (m, 2H), 4.22-4.10 (m, 2H), 4.05 (dd, J=12.6, 4.7 Hz, 1H), 3.85 (s, 3H), 3.58-3.48 (m, 2H), 3.30 (s, 3H), 3.28 (s, 1H), 3.24-3.18 (m, 1H), 3.09-3.03 (m, 2H), 3.01-2.96 (m, 1H), 2.93-2.91 (m, 1H), 2.85-2.73 (m, 6H), 2.71-2.61 (m, 7H), 2.54 (s, 3H), 2.17-2.04 (m, 1H), 1.99-1.94 (m, 1H), 1.91-1.85 (m, 2H), 1.82-1.73 (m, 2H), 1.63-1.61 (m, 2H), 1.50-1.39 (m, 1H). [M+H]⁺=945.4.

### Example 78: 3-(5-(2-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)ethyl)-3-fluoro-6-methylpyridin-2-yl)piperidine-2,6-dione

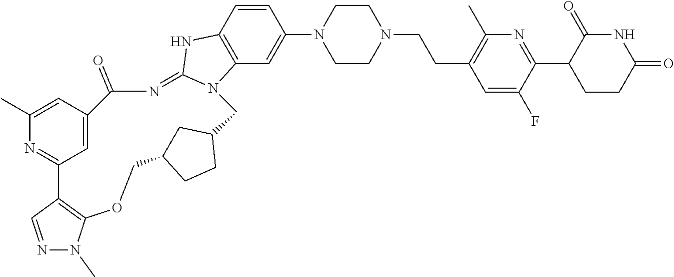

The title compound was prepared in a manner similar to that in Example 1 with intermediates 2 and 38.

¹H NMR (500 MHz, DMSO) δ 12.42 (s, 1H), 10.89 (s, 1H), 8.52 (s, 1H), 7.89 (s, 1H), 7.58 (d, J=10.7 Hz, 1H), 7.45 (s, 1H), 7.37 (d, J=8.7 Hz, 1H), 7.03 (s, 1H), 6.92 (d, J=8.6 Hz, 1H), 4.30 (s, 1H), 4.21-4.17 (m, 2H), 4.09 (s, 2H), 3.74 (s, 3H), 3.19 (s, 4H), 2.83-2.79 (m, 2H), 2.76-2.69 (m, 2H), 2.66 (s, 4H), 2.63-2.59 (m, 4H), 2.55 (s, 4H), 2.45 (s, 3H), 2.31-2.24 (m, 1H), 2.12-2.01 (m, 1H), 1.93-1.67 (m, 5H). [M+H]+=775.6.

### Example 79: 3-(5-((3S,4R)-3-fluoro-4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

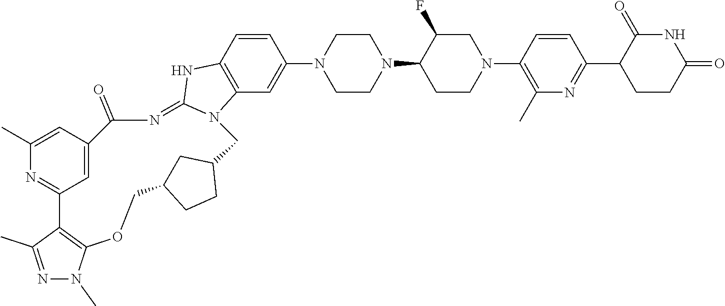

The title compound was prepared in a manner similar to that in Example 11 with intermediate 39 and 26.

¹H NMR (500 MHz, DMSO) δ 12.33 (s, 1H), 10.79 (s, 1H), 8.54 (s, 1H), 7.45 (s, 1H), 7.38 (dd, J=8.4, 4.6 Hz, 2H), 7.12 (d, J=8.1 Hz, 1H), 7.04 (s, 1H), 6.92 (d, J=8.7 Hz, 1H), 5.18-5.03 (m, 1H), 4.40-3.99 (m, 4H), 3.90 (dd, J=9.5, 5.3 Hz, 1H), 3.67 (s, 3H), 3.24-3.14 (m, 6H), 2.92-2.75 (m, 7H), 2.65-2.53 (m, 9H), 2.48 (s, 2H), 2.42 (s, 3H), 2.27-2.17 (m, 1H), 2.13-1.98 (m, 2H), 1.90-1.55 (m, 6H). [M+H]⁺=844.6.

### Example 80: (R)-3-(2,6-difluoro-4-(4-((S)-2-(methoxymethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

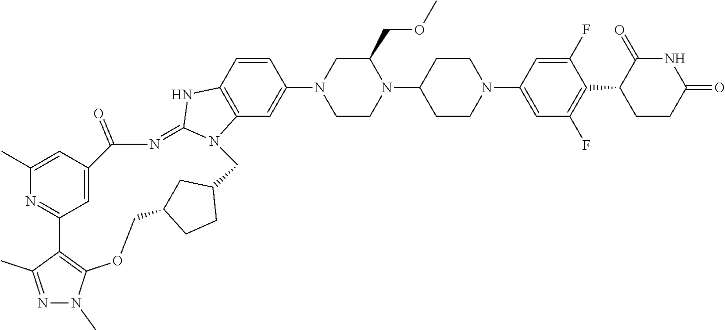

To a solution of intermediate 40 (85 mg, 0.15 mmol) and intermediate 7 (67 mg, 0.21 mmol) in DCM (6 mL) was added STAB (96 mg, 0.45 mmol). Then the mixture was stirred at RT for 48 hours. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=15:1), followed by Prep-HPLC chromatography to afford the title product (28 mg, 21.7%).

¹H NMR (500 MHz, DMSO) δ 12.42 (s, 1H), 10.87 (s, 1H), 8.54 (s, 1H), 7.45 (s, 1H), 7.37 (d, J=8.7 Hz, 1H), 6.98 (s, 1H), 6.87 (d, J=8.7 Hz, 1H), 6.64 (d, J=12.8 Hz, 2H), 4.31-4.03 (m, 5H), 3.82 (d, J=11.7 Hz, 2H), 3.67 (s, 3H), 3.57-3.52 (m, 2H), 3.31-3.27 (m, 4H), 3.20 (s, 1H), 3.08-2.88 (m, 4H), 2.83-2.54 (m, 12H), 2.48 (s, 3H), 2.13-2.04 (m, 1H), 1.97-1.71 (m, 7H), 1.62 (d, J=8.7 Hz, 2H), 1.45 (d, J=11.9 Hz, 1H). [M+H]⁺=891.6.

### Example 82: 3-(3-fluoro-5-((3S,4R)-3-fluoro-4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

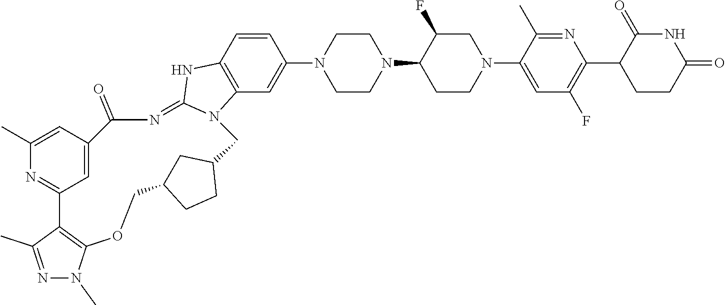

The title compound was prepared in a manner similar to that in Example 11 with intermediates 39 and 21.

¹H NMR (500 MHz, DMSO) δ 12.43 (s, 1H), 10.87 (s, 1H), 8.54 (s, 1H), 7.45 (s, 1H), 7.37 (d, J=8.9 Hz, 1H), 7.34 (s, 1H), 7.05 (s, 1H), 6.92 (d, J=8.0 Hz, 1H), 5.23-5.04 (m, 1H), 4.39-4.00 (m, 5H), 3.67 (s, 3H), 3.48-3.35 (m, 2H), 3.30-3.22 (m, 2H), 3.19 (d, J=2.6 Hz, 2H), 2.95-2.78 (m, 6H), 2.76-2.65 (m, 2H), 2.61-2.53 (m, 8H), 2.48 (s, 3H), 2.40 (s, 3H), 2.30-2.20 (m, 1H), 2.08-1.99 (m, 2H), 1.91-1.78 (m, 4H), 1.70-1.60 (m, 1H). [M+H]⁺=862.7.

### Example 83: 3-(5-((S)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3,3-difluoropiperidin-1-yl)-6-methylpyridin-2-yl)piperidine-2,6-dione

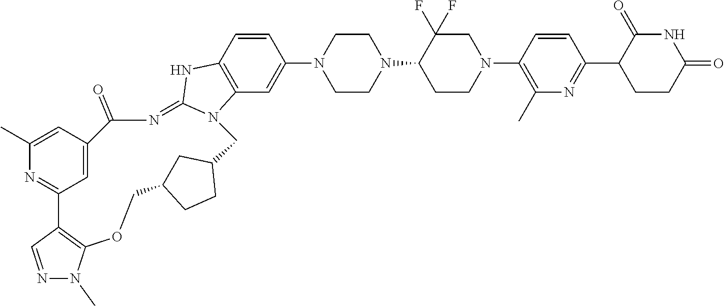

To a stirred solution of intermediate 1 (60 mg, 0.1 mmol) in DMA (5 mL) were added intermediate 41 (47 mg, 0.1 mmol), t-BuONa (44 mg, 0.5 mmol), Ruphos (21 mg, 0.05 mmol) and Pd₂(dba)₃ (46 mg, 0.02 mmol) at room temperature under nitrogen atmosphere. The resulting mixture was stirred for 15 min at 100° C. under nitrogen atmosphere. The mixture was allowed to cool down to room temperature. The resulting mixture was concentrated under reduced pressure. The residue was diluted with DCM (50 mL), washed with water (3×20 mL) and brine (20 mL). The organic layer was dried over anhydrous Na₂SO₄, filtered and concentrated under reduced pressure. The residue was purified by pre-HPLC to afford the title compound (8.97 mg, 9.2%).

¹H NMR (500 MHz, DMSO) δ 12.43 (s, 1H), 10.80 (s, 1H), 8.52 (s, 1H), 7.89 (s, 1H), 7.48-7.40 (m, 2H), 7.37 (d, J=8.7 Hz, 1H), 7.14 (d, J=8.2 Hz, 1H), 7.04 (s, 1H), 6.92 (d, J=8.7 Hz, 1H), 4.31 (s, 1H), 4.19 (s, 1H), 4.09 (s, 2H), 3.92 (dd, J=9.5, 5.4 Hz, 1H), 3.74 (s, 3H), 3.17 (s, 5H), 3.12-2.90 (m, 7H), 2.87-2.83 (m, 1H), 2.64-2.56 (m, 4H), 2.55-2.53 (m, 4H), 2.43 (s, 3H), 2.26-2.17 (m, 1H), 2.12-1.99 (m, 2H), 1.95-1.93 (m, 2H), 1.83-1.68 (m, 4H). [M+H]⁺=848.7.

### Example 84: (R)-3-(4-((3R,4S)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-2,6-difluorophenyl)piperidine-2,6-dione

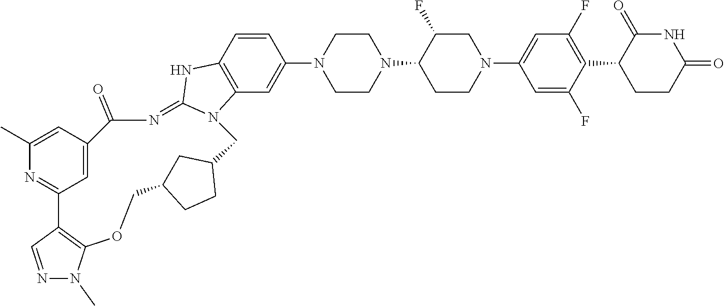

To the solution of intermediate 1 (100 mg, 0.19 mmol), intermediate 42 (94 mg, 0.23 mmol) and t-BuONa (73 mg, 0.76 mmol) in 6 mL DMA were added Pd₂(dba)₃ (35 mg, 0.038 mmol) and RuPhos (35 mg, 0.076 mmol). The mixture was stirred at 80° C. for 0.5 hour under N₂. The reaction was quenched with NH₄Cl/H₂O (15 mL) and extracted with DCM (3×15 mL). The combined organic phase was washed with brine (1×15 mL), dried over Na₂SO₄, filtered and concentrated in vacuum. The residue was purified by silica gel column (DCM:CH₃OH=15:1) to give the racemate, which was further purified by Prep chiral-HPLC to afford the title product (35 mg, 21.7%).

Chiral HPLC method: Column: CHIRALPAK IE, 2 cm×25 cm, 5 um

Mobile Phase A: MTBE

Mobile Phase B: MeOH:DCM=50:50

Flow rate: 20 mL/min

Gradient: 10% to 50% in 2.0 min, hold 1.0 min at 50%,

The title compound corresponds to peak A @ 4.083 min

¹H NMR (500 MHz, DMSO) δ 12.41 (s, 1H), 10.87 (s, 1H), 8.52 (s, 1H), 7.89 (s, 1H), 7.45 (s, 1H), 7.37 (d, J=8.7 Hz, 1H), 7.03 (s, 1H), 6.91 (d, J=8.8 Hz, 1H), 6.66 (d, J=12.8 Hz, 2H), 5.20-5.05 (m, 1H), 4.35-4.01 (m, 6H), 3.93 (d, J=11.7 Hz, 1H), 3.74 (s, 3H), 3.22-3.13 (m, 4H), 3.04-2.74 (m, 8H), 2.70-2.55 (m, 7H), 2.15-2.04 (m, 1H), 1.99-1.65 (m, 8H). [M+H]⁺=851.6.

### Example 85: (R)-3-(4-(4-((S)-4-((7¹R,7³S,E)-1³-(difluoromethyl)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

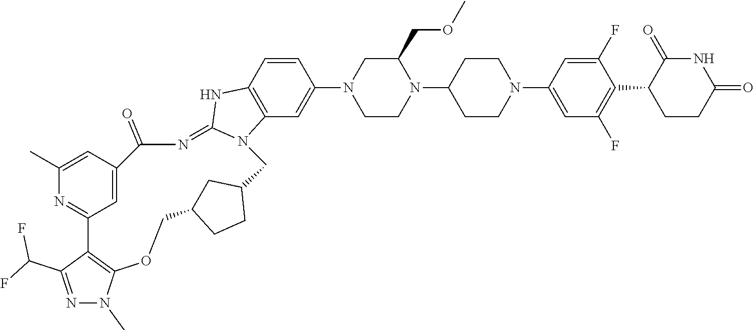

The title compound was prepared in a manner similar to that in Example 1 with intermediates 43 and 7.

¹H NMR (500 MHz, DMSO) δ 12.45 (s, 1H), 10.87 (s, 1H), 8.61 (s, 1H), 7.77 (t, J=54.5 Hz, 1H), 7.53 (s, 1H), 7.37 (d, J=8.7 Hz, 1H), 6.98 (s, 1H), 6.88 (d, J=8.8 Hz, 1H), 6.65 (s, 1H), 6.63 (s, 1H), 4.38-4.22 (m, 2H), 4.17-4.01 (m, 3H), 3.86-3.78 (m, 5H), 3.60-3.55 (m, 1H), 3.53-3.48 (m, 1H), 3.30 (s, 3H), 3.28-3.16 (m, 3H), 3.09-2.96 (m, 3H), 2.94-2.86 (m, 1H), 2.85-2.68 (m, 5H), 2.67-2.52 (m, 7H), 2.15-2.04 (m, 1H), 1.99-1.94 (m, 1H), 1.91-1.80 (m, 4H), 1.76-1.70 (m, 1H), 1.69-1.57 (m, 2H), 1.49-1.40 (m, 1H). [M+H]⁺=927.7.

### Example 86: (R)-3-(4-(3-(((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)methyl)azetidin-1-yl)-2,6-difluorophenyl)piperidine-2,6-dione

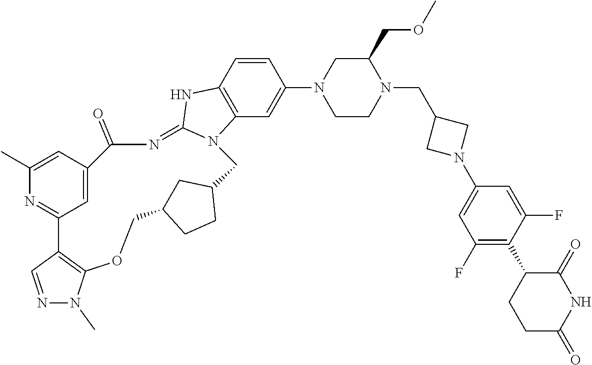

The title compound was prepared in a manner similar to that in Example 1 with intermediates 3 and 44.

¹H NMR (500 MHz, DMSO) δ 12.43 (s, 1H), 10.85 (s, 1H), 8.52 (s, 1H), 7.88 (d, J=6.0 Hz, 1H), 7.45 (s, 1H), 7.37 (d, J=8.7 Hz, 1H), 7.01 (s, 1H), 6.89 (d, J=8.6 Hz, 1H), 6.13 (s, 1H), 6.11 (s, 1H), 4.37-4.25 (m, 1H), 4.23-4.15 (m, 1H), 4.14-3.99 (m, 3H), 3.94 (q, J=7.8 Hz, 2H), 3.74 (s, 3H), 3.59 (dd, J=9.8, 4.2 Hz, 1H), 3.55-3.43 (m, 3H), 3.40-3.35 (m, 1H), 3.28 (s, 3H), 3.07-3.00 (m, 1H), 3.00-2.89 (m, 3H), 2.88-2.66 (m, 4H), 2.65-2.57 (m, 3H), 2.55 (s, 3H), 2.51 (s, 2H), 2.49-2.40 (m, 2H), 2.13-2.02 (m, 1H), 1.97-1.90 (m, 2H), 1.87-1.78 (m, 2H), 1.72-1.62 (m, 1H). [M+H]⁺=863.7.

### Example 87: (R)-3-(2,6-difluoro-4-(4-((S)-4-((7¹R,7³S,E)-5⁵-fluoro-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)phenyl)piperidine-2,6-dione

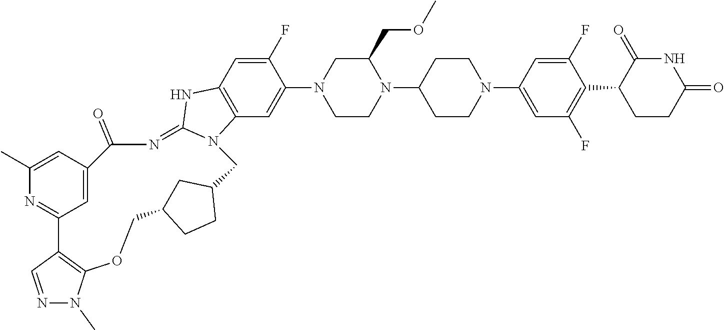

To a solution of intermediate 45 (60 mg, 0.1 mmol)) and intermediate 7 (64 mg, 0.2 mmol) in DCE (6 mL) was added STAB (64 mg, 0.3 mmol). Then the mixture was stirred at RT for 48 hours. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=15:1), followed by Prep-HPLC chromatography to afford the title product (17 mg, 19%).

¹H NMR (500 MHz, DMSO) δ 12.55 (s, 1H), 10.87 (s, 1H), 8.51 (s, 1H), 7.89 (s, 1H), 7.44 (s, 1H), 7.29 (d, J=11.5 Hz, 1H), 7.17 (d, J=7.2 Hz, 1H), 6.64 (d, J=12.8 Hz, 2H), 4.37-4.26 (m, 1H), 4.19 (d, J=8.0 Hz, 1H), 4.10-4.06 (m, 3H), 3.81 (d, J=11.9 Hz, 2H), 3.74 (s, 3H), 3.64-3.53 (m, 2H), 3.29 (s, 3H), 3.06 (dd, J=14.5, 10.1 Hz, 2H), 3.01 (s, 2H), 2.92-2.89 (m, 2H), 2.79-2.75 (m, 4H), 2.72-2.66 (m, 1H), 2.65-2.62 (m, 2H), 2.55 (s, 3H), 2.54-2.50 (m, 3H), 2.15-2.04 (m, 1H), 1.99-1.86 (m, 3H), 1.85-1.72 (m, 3H), 1.63-1.60 (m, 2H), 1.49-1.45 (m, 1H). [M+H]⁺=895.2.

### Example 88: 3-(4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-fluorophenoxy)piperidine-2,6-dione

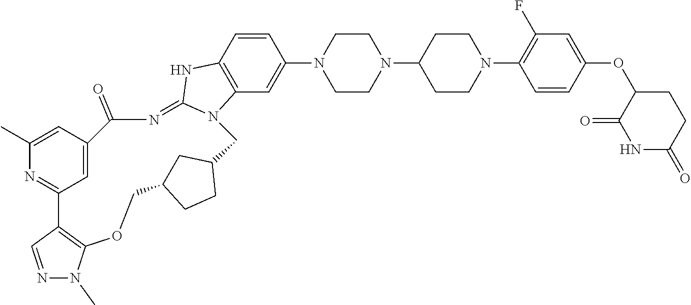

To a solution of intermediate 2 (50 mg, 0.1 mmol), intermediate 46 (61 mg, 0.2 mmol) in DCE (5 mL) was added STAB (60 mg, 0.3 mmol). Then the mixture was stirred at 50° C. for 1 hr. H₂O (10 mL) was added and the resulting mixture was extracted with DCM (10 mL×3). The combined organic phase was washed with brine (10 mL), dried over Na₂SO₄, filtered and concentrated under reduced pressure. The residue was purified by prep-HPLC to afford the title compound (3.7 mg, 4.7%).

¹H NMR (500 MHz, DMSO) δ 12.41 (s, 1H), 10.92 (s, 1H), 8.52 (s, 1H), 7.89 (s, 1H), 7.45 (s, 1H), 7.37 (d, J=8.6 Hz, 1H), 7.03-6.97 (m, 2H), 6.94-6.86 (m, 2H), 6.78-6.77 (m, 1H), 5.14-5.11 (m, 1H), 4.30 (s, 1H), 4.19 (s, 1H), 4.10 (s, 2H), 3.74 (s, 3H), 3.18 (s, 4H), 2.76-2.67 (m, 5H), 2.67-2.57 (m, 6H), 2.55 (s, 5H), 2.38-2.36 (m, 1H), 2.21-2.08 (m, 2H), 1.95-1.75 (m, 6H), 1.70-1.57 (m, 3H). [M+H]⁺=831.7.

### Example 89: 3-(3-fluoro-5-(4-((S)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

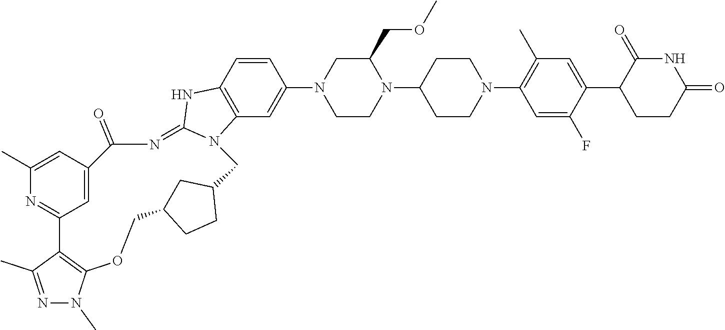

The title compound was prepared in a manner similar to that in Example 1 with intermediates 40 and 12.

¹H NMR (500 MHz, DMSO) δ 12.39 (s, 1H), 10.87 (s, 1H), 8.54 (s, 1H), 7.45 (s, 1H), 7.38 (d, J=8.7 Hz, 1H), 7.33 (d, J=11.6 Hz, 1H), 6.99 (s, 1H), 6.92-6.86 (m, 1H), 4.35-3.96 (m, 5H), 3.67 (s, 3H), 3.59-3.48 (m, 2H), 3.31-3.16 (m, 8H), 3.07-2.85 (m, 5H), 2.75-2.53 (m, 11H), 2.48 (s, 2H), 2.38 (s, 3H), 2.30-2.20 (m, 1H), 2.08-2.01 (m, 1H), 1.97-1.54 (m, 9H). [M+H]⁺=888.6.

### Example 90: 3-(3-fluoro-5-(4-((R)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

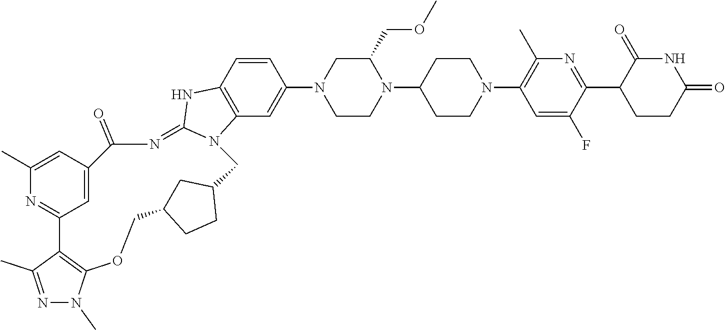

The title compound was prepared in a manner similar to that in Example 1 with intermediates 47 and 12.

¹H NMR (500 MHz, DMSO) δ 12.39 (s, 1H), 10.87 (s, 1H), 8.54 (s, 1H), 7.45 (s, 1H), 7.38 (d, J=8.7 Hz, 1H), 7.33 (d, J=11.6 Hz, 1H), 6.99 (s, 1H), 6.92-6.86 (m, 1H), 4.35-3.96 (m, 5H), 3.67 (s, 3H), 3.59-3.48 (m, 2H), 3.31-3.16 (m, 8H), 3.07-2.85 (m, 5H), 2.75-2.53 (m, 11H), 2.48 (s, 2H), 2.38 (s, 3H), 2.30-2.20 (m, 1H), 2.08-2.01 (m, 1H), 1.97-1.54 (m, 9H). [M+H]⁺=888.6.

### Example 91: (R)-3-(2,6-difluoro-4-(4-((R)-2-(methoxymethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

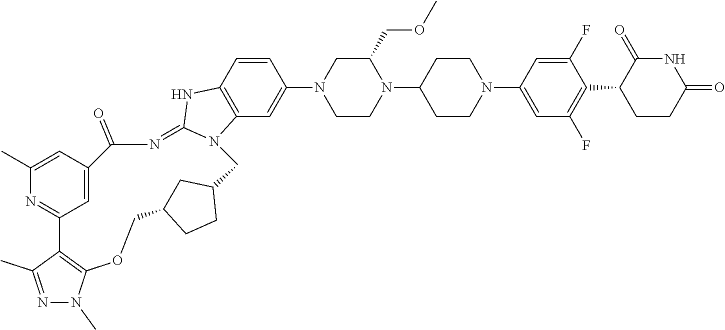

To the solution of intermediate 47 (65 mg, 0.11 mmol) and intermediate 7 (72 mg, 0.22 mmol) in 2 mL DCE was added STAB (71 mg, 0.33 mmol). The mixture was stirred at 70° C. for 4 days. The mixture was concentrated and purified by silica column chromatography (MeOH:DCM=0-10%) to afford the crude product. The crude product was purified by prep-HPLC to afford title compound (7.0 mg, 0.008 mmol, 7.1%).

¹H NMR (500 MHz, DMSO) δ 12.43 (s, 1H), 10.87 (s, 1H), 8.53 (s, 1H), 7.45 (s, 1H), 7.37 (d, J=8.7 Hz, 1H), 6.97 (s, 1H), 6.86 (dd, J=8.8, 1.8 Hz, 1H), 6.64 (d, J=12.7 Hz, 2H), 4.38-3.97 (m, 5H), 3.82 (d, J=12.4 Hz, 2H), 3.67 (s, 3H), 3.61-3.55 (m, 1H), 3.54-3.48 (m, 1H), 3.31-3.17 (m, 6H), 3.06-2.97 (m, 3H), 2.93-2.87 (m, 1H), 2.84-2.63 (m, 6H), 2.61-2.51 (m, 6H), 2.48 (s, 3H), 2.14-2.05 (m, 1H), 1.99-1.93 (m, 1H), 1.91-1.70 (m, 6H), 1.65-1.60 (m, 1H), 1.49-1.40 (m, 1H). [M+H]⁺=891.2.

### Example 92: 3-((4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3,5-difluorophenyl)amino)piperidine-2,6-dione

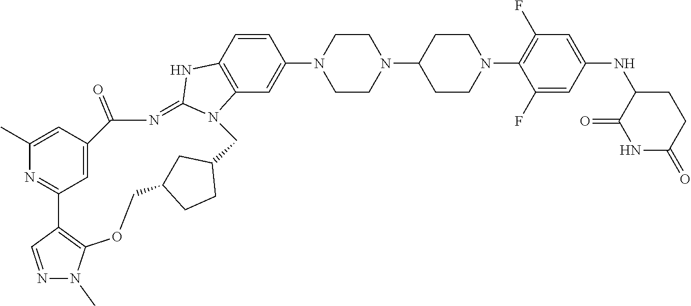

The title compound was prepared in a manner similar to that in Example 1 with intermediates 2 and 48.

¹H NMR (500 MHz, DMSO) δ 12.42 (s, 1H), 10.81 (s, 1H), 8.52 (s, 1H), 7.89 (s, 1H), 7.45 (s, 1H), 7.37 (d, J=8.7 Hz, 1H), 7.02 (s, 1H), 6.91 (d, J=7.2 Hz, 1H), 6.33 (s, 1H), 6.31 (s, 1H), 6.24 (d, J=7.9 Hz, 1H), 4.37-4.03 (m, 5H), 3.74 (s, 3H), 3.18 (s, 4H), 3.07-2.95 (m, 4H), 2.78-2.57 (m, 8H), 2.55 (s, 3H), 2.43-2.36 (m, 1H), 2.10-2.03 (m, 1H), 2.02-1.97 (m, 1H), 1.96-1.79 (m, 6H), 1.72-1.63 (m, 1H), 1.63-1.53 (m, 2H), 1.52-1.40 (m, 1H). [M+H]⁺=848.7.

### Example 93: 3-((4-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-3,5-difluorophenyl)amino)piperidine-2,6-dione

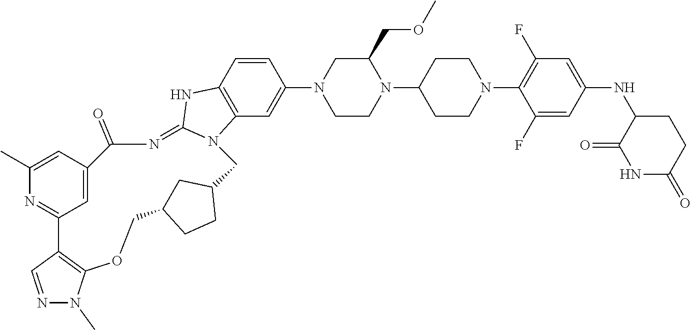

To a solution of intermediate 3 (80.0 mg, 0.14 mmol) and intermediate 48 (70.9 mg, 0.21 mmol) in DCE (5 mL) was added STAB (59.4 mg, 0.28 mmol) at 50° C. The mixture was stirred at 50° C. for 16 hr. Water (10 mL) was poured into the mixture. Then the mixture was extracted with DCM/MeOH (10:1, 20 mL). The organic phase was washed with brine (10 mL), dried over Na₂SO₄, filtered and concentrated in vacuum. The residue was purified by prep-HPLC (C-18 column chromatography (0.1% FA in water:acetonitrile=90:10˜60:40 gradient elution) to afford the product (28.0 mg, 22.4%).

¹H NMR (500 MHz, DMSO) δ 12.42 (s, 1H), 10.81 (s, 1H), 8.52 (s, 1H), 7.89 (s, 1H), 7.45 (s, 1H), 7.37 (d, J=8.6 Hz, 1H), 6.98 (s, 1H), 6.88 (d, J=8.3 Hz, 1H), 6.33 (s, 1H), 6.31 (s, 1H), 6.23 (d, J=7.8 Hz, 1H), 4.37-4.26 (m, 2H), 4.23-4.16 (m, 1H), 4.15-3.98 (m, 2H), 3.74 (s, 3H), 3.56 (dd, J=9.5, 4.1 Hz, 1H), 3.48 (dd, J=9.6, 5.9 Hz, 1H), 3.38-3.32 (m, 6H), 3.05-2.92 (m, 6H), 2.92-2.87 (m, 2H), 2.83-2.63 (m, 4H), 2.62-2.57 (m, 2H), 2.55 (s, 3H), 2.12-2.03 (m, 1H), 2.00-1.63 (m, 9H), 1.56-1.45 (m, 1H). [M+H]⁺=892.7.

### Example 94: (R)-3-(4-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-3-(hydroxymethyl)piperazin-1-yl)piperidin-1-yl)-2,6-difluorophenyl)piperidine-2,6-dione

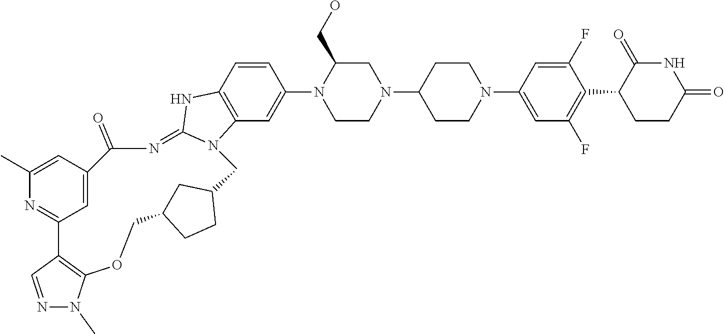

The titled compound was prepared in a manner similar to that in Example 1 with intermediates 49 and 7.

¹H NMR (500 MHz, DMSO) δ 12.40 (s, 1H), 10.87 (s, 1H), 8.52 (s, 1H), 7.89 (s, 1H), 7.45 (s, 1H), 7.35 (d, J=8.7 Hz, 1H), 6.96 (s, 1H), 6.88 (d, J=7.2 Hz, 1H), 6.66 (s, 1H), 6.64 (s, 1H), 4.36-4.27 (m, 1H), 4.21-4.18 (m, 1H), 4.08-4.02 (m, 3H), 3.81 (s, 1H), 3.79 (s, 1H), 3.74 (s, 4H), 3.69-3.66 (s, 1H), 3.25-3.21 (m, 2H), 3.10 (d, J=8.8 Hz, 1H), 2.99-2.97 (m, 1H), 2.96-2.92 (m, 1H), 2.82-2.79 (m, 3H), 2.76-2.74 (m, 1H), 2.64 (s, 1H), 2.59 (s, 2H), 2.55 (s, 5H), 2.46-2.42 (m, 2H), 2.36 (s, 1H), 2.10-2.07 (m, 1H), 1.99-1.95 (m, 2H), 1.87-1.83 (m, 4H), 1.68-1.63 (m, 1H), 1.53-1.50 (m, 2H); [M+H]⁺=863.5.

### Example 95: (R)-3-(4-(4-((R)-4-((7¹R,7³S,E)-1¹,2⁶-dimethyl-3-oxo-1³-(trifluoromethyl)-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

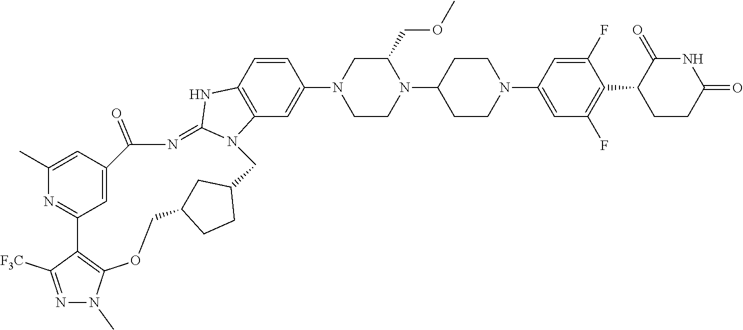

The titled compound was prepared in a manner similar to that in Example 1 with intermediates 50 and 7.

¹H NMR (500 MHz, DMSO) δ 12.45 (s, 1H), 10.87 (s, 1H), 8.61 (s, 1H), 7.59 (s, 1H), 7.37 (d, J=8.7 Hz, 1H), 6.99 (s, 1H), 6.88 (d, J=7.8 Hz, 1H), 6.64 (d, J=12.7 Hz, 2H), 4.41-4.24 (m, 2H), 4.22-4.10 (m, 2H), 4.05 (dd, J=12.6, 4.7 Hz, 1H), 3.85 (s, 3H), 3.58-3.48 (m, 2H), 3.30 (s, 3H), 3.28 (s, 1H), 3.24-3.18 (m, 1H), 3.09-3.03 (m, 2H), 3.01-2.96 (m, 1H), 2.93-2.91 (m, 1H), 2.85-2.73 (m, 6H), 2.71-2.61 (m, 7H), 2.54 (s, 3H), 2.17-2.04 (m, 1H), 1.99-1.94 (m, 1H), 1.91-1.85 (m, 2H), 1.82-1.73 (m, 2H), 1.63-1.61 (m, 2H), 1.50-1.39 (m, 1H). [M+H]⁺=945.4.

### Example 96: (R)-3-(2,6-difluoro-4-(4-((S)-2-(methoxymethyl)-4-((7R,7³S,E)-1¹,2⁶,5⁵-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

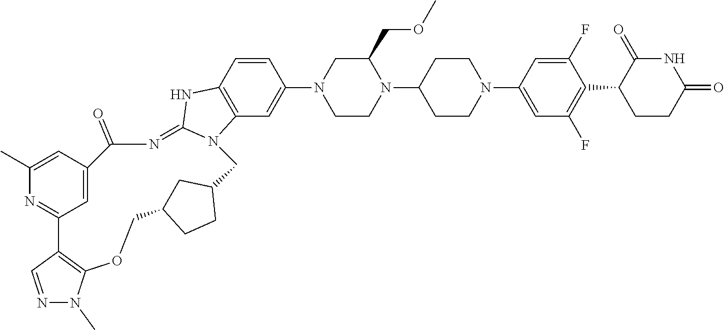

To a solution of intermediate 51 (80 mg, 0.14 mmol) and intermediate 7 (57 mg, 0.18 mmol) in DCE (6 mL) was added sodium triacetoxyborohydride (72 mg, 0.34 mmol) at room temperature. The resulting mixture was stirred at 50° C. overnight. The reaction was quenched with saturated aq. NaHCO₃ (20 mL) and extracted with DCM (2×30 mL). The combined organic layer was washed with brine (2×30 mL), dried over Na₂SO₄ and concentrated under vacuum to afford the crude residue, which was purified by silica gel column chromatography (DCM:MeOH=100:0-90:10 gradient elution) to give the crude product, which was purified by Prep-HPLC to afford the title compound (15.2 mg, 12.3%).

The titled compound was prepared in a manner similar to that in Example 1 with intermediates 51 and 7.

¹H NMR (500 MHz, DMSO) δ 12.45 (s, 1H), 10.87 (s, 1H), 8.52 (s, 1H), 7.89 (s, 1H), 7.44 (s, 1H), 7.32 (s, 1H), 7.22 (s, 1H), 6.66 (s, 1H), 6.63 (s, 1H), 4.30 (s, 1H), 4.18 (d, J=8.2 Hz, 1H), 4.12-4.06 (m, 3H), 3.80 (d, J=12.0 Hz, 2H), 3.74 (s, 3H), 3.67-3.61 (m, 2H), 3.32-3.31 (m, 2H), 3.28 (s, 3H), 3.06 (s, 1H), 2.88 (s, 4H), 2.81-2.76 (m, 5H), 2.72-2.69 (m, 1H), 2.59 (s, 2H), 2.55 (s, 3H), 2.33 (s, 3H), 2.08 (t, J=11.1 Hz, 1H), 1.97 (d, J=5.5 Hz, 1H), 1.91 (s, 2H), 1.83 (s, 2H), 1.77 (s, 2H), 1.67 (s, 1H), 1.59 (d, J=9.9 Hz, 1H), 1.47 (d, J=11.0 Hz, 1H); [M+H]⁺=891.5

### Example 98: 3-((5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)amino)piperidine-2,6-dione

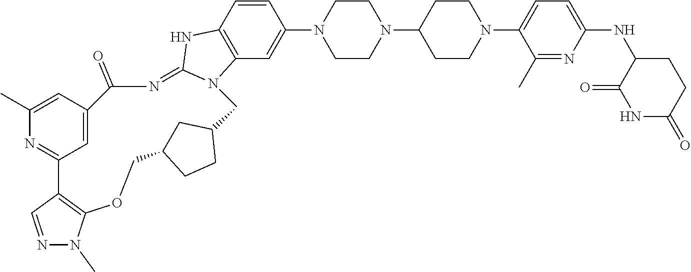

The title compound was prepared in a manner similar to that in Example 1 with intermediates 2 and 52.

¹H NMR (500 MHz, DMSO) δ 12.42 (s, 1H), 10.73 (s, 1H), 8.52 (s, 1H), 7.89 (s, 1H), 7.45 (s, 1H), 7.37 (d, J=8.7 Hz, 1H), 7.24 (d, J=8.7 Hz, 1H), 7.02 (s, 1H), 6.91 (dd, J=8.8, 1.7 Hz, 1H), 6.40 (dd, J=19.4, 8.2 Hz, 2H), 4.70-4.62 (m, 1H), 4.35-4.04 (m, 4H), 3.74 (s, 3H), 3.20-3.15 (m, 4H), 2.93 (d, J=10.4 Hz, 2H), 2.79-2.68 (m, 5H), 2.65-2.53 (m, 9H), 2.40-2.32 (m, 1H), 2.27 (s, 3H), 2.11-1.76 (m, 8H), 1.59 (d, J=9.3 Hz, 3H). [M+H]⁺=827.6.

### Example 99: 3-((4-(4-(4-((7¹R,7³S,E)-1³-(difluoromethyl)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3,5-difluorophenyl)amino)piperidine-2,6-dione

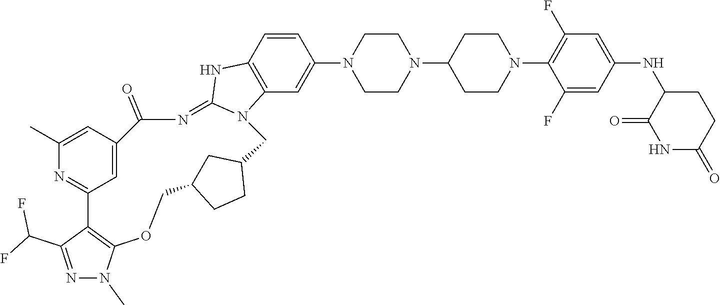

The title compound was prepared in a manner similar to that in Example 1 with intermediates 53 and 48.

¹H NMR (500 MHz, DMSO) δ 12.45 (s, 1H), 10.81 (s, 1H), 8.61 (s, 1H), 7.77 (t, J=54.6 Hz, 1H), 7.54 (s, 1H), 7.37 (d, J=8.7 Hz, 1H), 7.03 (s, 1H), 6.92 (d, J=8.3 Hz, 1H), 6.33 (s, 1H), 6.31 (s, 1H), 6.24 (d, J=8.0 Hz, 1H), 4.40-4.21 (m, 3H), 4.21-4.00 (m, 2H), 3.82 (s, 3H), 3.22-3.13 (m, 4H), 3.05-2.95 (m, 4H), 2.79-2.54 (m, 12H), 2.41-2.34 (m, 1H), 2.09-2.01 (m, 1H), 1.97-1.71 (m, 7H), 1.71-1.62 (m, 1H), 1.61-1.51 (m, 2H). [M+H]⁺=898.7.

### Example 100: 3-(5-(4-(4-((7¹R,7³S,E)-5⁵-fluoro-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4-methylpyridin-2-yl)piperidine-2,6-dione

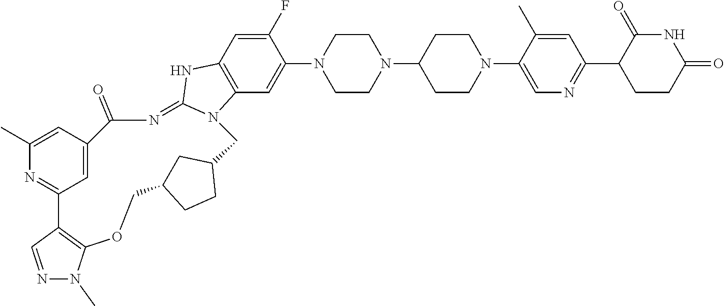

The title compound was prepared in a manner similar to that in Example 1 with intermediates 54 and 14.

¹H NMR (500 MHz, DMSO) δ 12.42 (s, 1H), 10.73 (s, 1H), 8.45 (s, 1H), 8.08 (s, 1H), 7.82 (s, 1H), 7.38 (s, 1H), 7.23 (d, J=11.3 Hz, 1H), 7.12 (d, J=5.6 Hz, 1H), 7.07 (s, 1H), 4.41-4.24 (m, 2H), 4.22-4.10 (m, 2H), 4.05 (dd, J=12.6, 4.7 Hz, 1H), 3.58-3.48 (m, 2H), 3.30 (s, 3H), 3.28 (s, 1H), 3.24-3.18 (m, 1H), 3.09-3.03 (m, 2H), 3.01-2.96 (m, 1H), 2.93-2.91 (m, 1H), 2.85-2.73 (m, 6H), 2.71-2.61 (m, 6H), 2.54 (s, 3H), 2.21 (s, 3H), 2.17-2.04 (m, 1H), 1.99-1.94 (m, 1H), 1.91-1.85 (m, 2H), 1.82-1.73 (m, 2H), 1.63-1.61 (m, 2H), 1.50-1.39 (m, 1H). [M+H]⁺=830.2.

### Example 101: 3-(5-((S)-3,3-difluoro-4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4-methylpyridin-2-yl)piperidine-2,6-dione

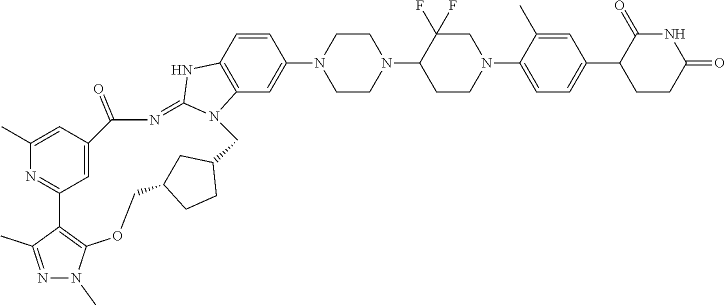

The title compound was prepared in a manner similar to that in Example 11 with intermediates 39 and 56.

¹H NMR (500 MHz, DMSO) δ 12.31 (s, 1H), 10.81 (s, 1H), 8.54 (s, 1H), 8.19 (s, 1H), 7.45 (s, 1H), 7.37 (d, J=8.7 Hz, 1H), 7.18 (s, 1H), 7.04 (s, 1H), 6.95-6.89 (m, 1H), 4.35-3.98 (m, 4H), 3.92 (dd, J=9.0, 5.4 Hz, 1H), 3.67 (s, 3H), 3.24-3.05 (m, 8H), 3.02-2.90 (m, 6H), 2.65-2.53 (m, 8H), 2.48 (s, 2H), 2.29 (s, 3H), 2.20 (dd, J=9.0, 4.5 Hz, 1H), 2.10 (dd, J=13.5, 5.8 Hz, 1H), 2.03-1.58 (m, 7H). [M+H]⁺=862.6.

### Example 102: 3-(5-((3S,4R)-3-fluoro-4-(4-((7¹R,7³S,E)-1¹,2⁶,5⁵-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)piperidine-2,6-dione

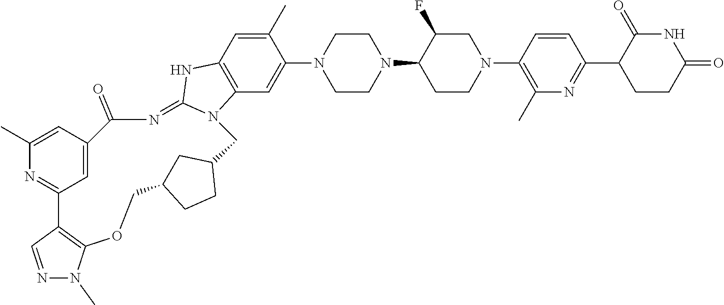

The titled compound was prepared in a manner similar to that in Example 11 with intermediates 57 and 26.

¹H NMR (500 MHz, DMSO) δ 12.45 (s, 1H), 10.79 (s, 1H), 8.52 (s, 1H), 7.89 (s, 1H), 7.45 (s, 1H), 7.39 (d, J=8.2 Hz, 1H), 7.33 (s, 1H), 7.24 (s, 1H), 7.12 (d, J=8.2 Hz, 1H), 4.32 (s, 1H), 4.19 (t, J=8.0 Hz, 1H), 4.10 (s, 2H), 3.90 (dd, J=9.5, 5.2 Hz, 1H), 3.74 (s, 3H), 3.39-3.36 (m, 2H), 3.31-3.29 (m, 2H), 3.19 (d, J=10.8 Hz, 1H), 2.93 (d, J=4.5 Hz, 4H), 2.86-2.81 (m, 5H), 2.66-2.62 (m, 1H), 2.60-2.57 (m, 4H), 2.55 (s, 4H), 2.43 (s, 3H), 2.34 (s, 3H), 2.29-2.18 (m, 1H), 2.11-2.01 (m, 2H), 1.96-1.87 (m, 1H), 1.85-1.83 (s, 3H), 1.69 (s, 1H); [M+H]⁺=844.5

### Example 103: 3-((3-chloro-4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)amino)piperidine-2,6-dione

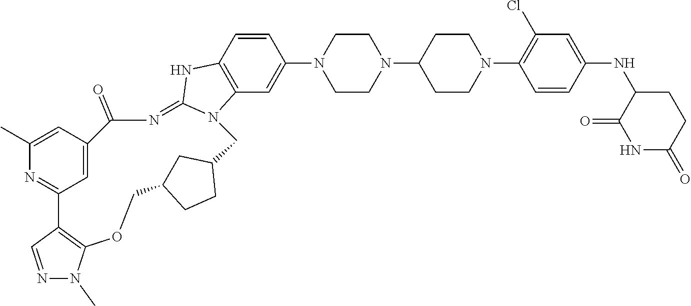

To a solution of intermediate 2 and intermediate 55 (74 mg, 0.22 mmol) in DCE (6 mL) was added STAB (70 mg, 0.33 mmol). Then the mixture was stirred at 50° C. for 16 hours. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=15:1), followed by Prep-HPLC chromatography to afford the title product (15 mg, 16%). 1H NMR (500 MHz, DMSO) δ 12.38 (s, 1H), 10.77 (s, 1H), 8.52 (s, 1H), 7.89 (s, 1H), 7.45 (s, 1H), 7.37 (d, J=8.7 Hz, 1H), 7.03 (s, 1H), 6.96-6.89 (m, 2H), 6.76 (d, J=2.5 Hz, 1H), 6.60 (dd, J=8.8, 2.4 Hz, 1H), 5.85 (d, J=7.7 Hz, 1H), 4.36-4.26 (m, 2H), 4.19 (t, J=8.4 Hz, 1H), 4.09 (s, 2H), 3.74 (s, 3H), 3.22-3.10 (m, 6H), 2.78-2.67 (m, 6H), 2.64-2.59 (m, 4H), 2.58-2.54 (m, 5H), 2.36 (s, 1H), 2.14-2.03 (m, 1H), 1.98-1.82 (m, 6H), 1.72-1.56 (m, 3H). [M+H]⁺=846.2.

### Example 104: (R)-3-(4-(4-((R)-4-((7¹R,7³S,E)-1³-(difluoromethyl)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

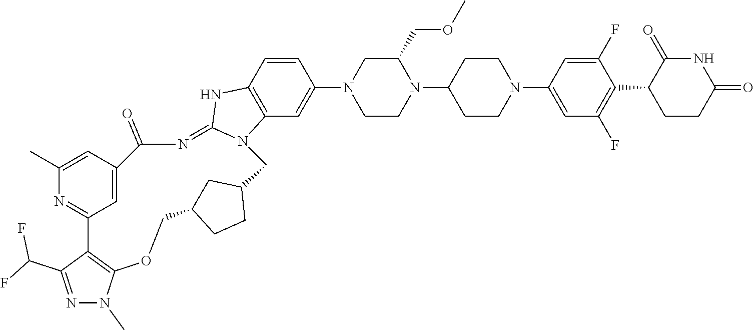

The title compound was prepared in a manner similar to that in Example 1 with intermediates 58 and 7.

¹H NMR (500 MHz, DMSO) δ 12.44 (s, 1H), 10.87 (s, 1H), 8.61 (s, 1H), 7.77 (t, J=54.6 Hz, 1H), 7.53 (s, 1H), 7.37 (d, J=8.7 Hz, 1H), 6.98 (s, 1H), 6.92-6.84 (m, 1H), 6.65 (s, 1H), 6.63 (s, 1H), 4.31 (dt, J=46.5, 14.9 Hz, 2H), 4.17-4.00 (m, 3H), 3.87-3.78 (m, 5H), 3.57 (dd, J=9.5, 4.0 Hz, 1H), 3.51 (dd, J=9.5, 5.5 Hz, 1H), 3.30 (s, 3H), 3.30-3.24 (m, 2H), 3.23-3.17 (m, 1H), 3.10-2.96 (m, 3H), 2.94-2.85 (m, 1H), 2.85-2.51 (m, 11H), 2.15-2.04 (m, 1H), 1.99-1.57 (m, 9H), 1.50-1.39 (m, 1H). [M+H]⁺=927.7.

### Example 105: 3-(5-((R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3,3-difluoropiperidin-1-yl)-6-methylpyridin-2-yl)piperidine-2,6-dione

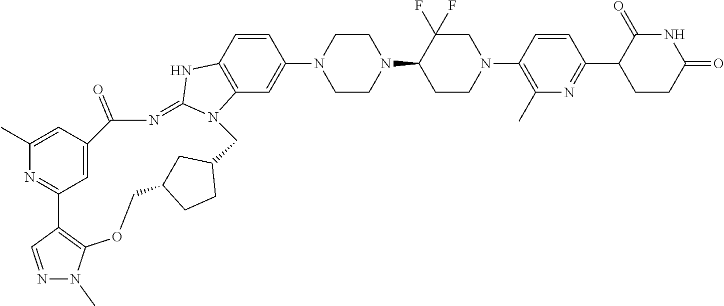

The title compound was prepared in a manner similar to that in Example 11 with intermediates 1 and 25.

¹H NMR (500 MHz, DMSO) δ 12.42 (s, 1H), 10.80 (s, 1H), 8.52 (s, 1H), 7.89 (s, 1H), 7.45 (s, 1H), 7.43 (d, J=8.3 Hz, 1H), 7.37 (d, J=8.7 Hz, 1H), 7.14 (d, J=8.1 Hz, 1H), 7.04 (s, 1H), 6.92 (dd, J=8.8, 1.8 Hz, 1H), 4.38-4.24 (m, 1H), 4.23-4.15 (m, 1H), 4.13-4.00 (m, 2H), 3.92 (dd, J=9.6, 5.3 Hz, 1H), 3.74 (s, 3H), 3.23-3.14 (m, 5H), 3.12-2.92 (m, 6H), 2.89-2.81 (m, 1H), 2.69-2.51 (m, 10H), 2.43 (s, 3H), 2.27-2.17 (m, 1H), 2.12-1.90 (m, 4H), 1.87-1.77 (m, 2H), 1.73-1.61 (m, 1H). [M+H]⁺=848.7.

### Example 106: 3-(5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-4-(methoxymethyl)piperidin-1-yl)-6-methylpyridin-2-yl)piperidine-2,6-dione

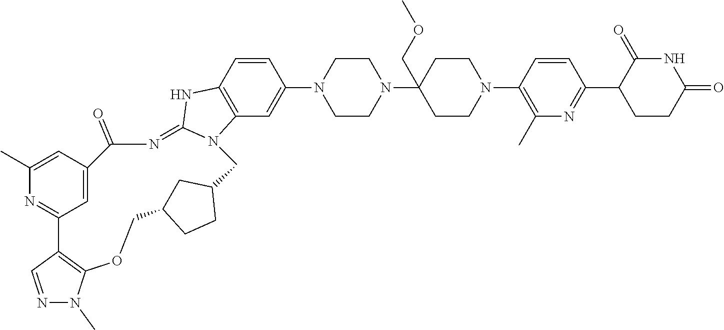

The title compound was prepared in a manner similar to that in Example 11 with intermediates 1 and 59.

¹H NMR (500 MHz, DMSO) δ 12.37 (s, 1H), 10.77 (s, 1H), 8.52 (s, 1H), 7.89 (s, 1H), 7.45 (s, 1H), 7.39 (d, J=8.3 Hz, 1H), 7.37 (d, J=8.8 Hz, 1H), 7.09 (d, J=8.2 Hz, 1H), 7.00 (s, 1H), 6.92 (d, J=8.7 Hz, 1H), 4.37-4.25 (m, 1H), 4.24-4.16 (m, 1H), 4.15-3.99 (m, 2H), 3.88 (dd, J=9.4, 5.3 Hz, 1H), 3.74 (s, 3H), 3.41 (s, 2H), 3.29 (s, 3H), 3.18-3.10 (m, 4H), 3.05-2.97 (m, 2H), 2.91-2.83 (m, 4H), 2.79-2.74 (m, 2H), 2.65-2.52 (m, 9H), 2.39 (s, 3H), 2.24-2.16 (m, 1H), 2.10-2.03 (m, 1H), 1.97-1.89 (m, 3H), 1.87-1.79 (m, 2H), 1.78-1.68 (m, 3H). [M+H]⁺=856.7.

### Example 108: (R)-3-(4-(4-(3-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)azetidin-1-yl)piperidin-1-yl)-2,6-difluorophenyl)piperidine-2,6-dione

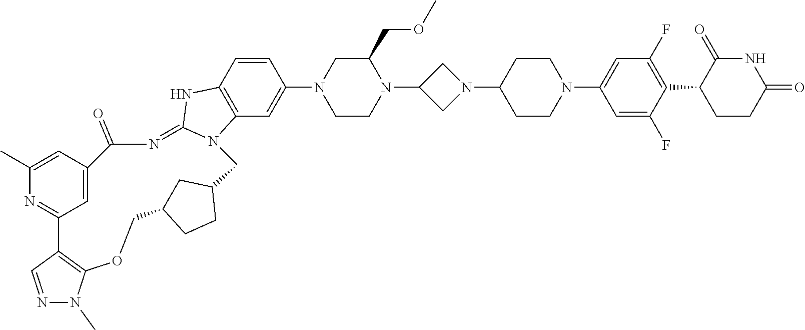

The title compound was prepared in a manner similar to that in Example 1 with intermediates 61 and 7.

¹H NMR (500 MHz, DMSO) δ 12.43 (s, 1H), 10.87 (s, 1H), 8.52 (s, 1H), 7.89 (s, 1H), 7.45 (s, 1H), 7.37 (d, J=8.7 Hz, 1H), 6.98 (s, 1H), 6.88 (dd, J=8.8, 1.7 Hz, 1H), 6.62 (d, J=12.8 Hz, 2H), 4.34-4.01 (m, 5H), 3.74 (s, 3H), 3.60 (s, 2H), 3.51-3.39 (m, 4H), 3.28-3.16 (m, 7H), 3.13-2.98 (m, 2H), 2.90-2.74 (m, 6H), 2.72-2.53 (m, 8H), 2.36 (s, 1H), 2.25-2.04 (m, 2H), 2.00-1.61 (m, 9H). [M+H]⁺=932.6.

### Example 111: 3-(4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-1)piperazin-1-yl)piperidin-1-yl)-3,5-difluorophenoxy)piperidine-2,6-dione

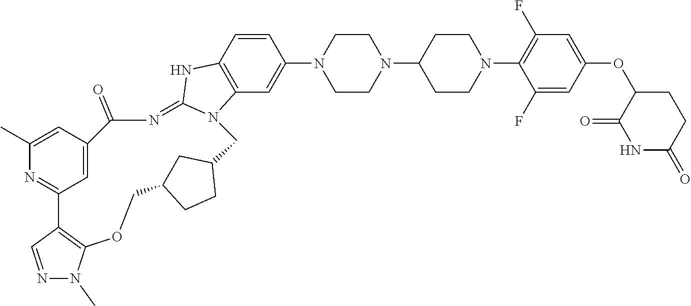

The titled compound was prepared in a manner similar to that in Example 1 with intermediates 2 and 62.

¹H NMR (500 MHz, DMSO) δ 12.42 (s, 1H), 10.96 (s, 1H), 8.52 (s, 1H), 7.89 (s, 1H), 7.45 (s, 1H), 7.37 (d, J=8.7 Hz, 1H), 7.03 (s, 1H), 6.91 (d, J=8.8 Hz, 1H), 6.79 (s, 1H), 6.77 (s, 1H), 5.23-5.19 (m, 1H), 4.30 (s, 1H), 4.18 (d, J=7.6 Hz, 1H), 4.10 (s, 2H), 3.74 (s, 3H), 3.17 (s, 4H), 3.12-3.11 (m, 2H), 3.05-3.00 (m, 2H), 2.77-2.67 (m, 5H), 2.67-2.57 (m, 4H), 2.55 (s, 3H), 2.43-2.36 (m, 1H), 2.23-2.08 (m, 2H), 1.91 (s, 1H), 1.85-1.83 (m, 5H), 1.67 (s, 1H), 1.63-1.53 (m, 2H). [M+H]⁺=849.7.

### Example 112: (R)-3-(4-((1-(1-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)azetidin-3-yl)amino)-2,6-difluorophenyl)piperidine-2,6-dione

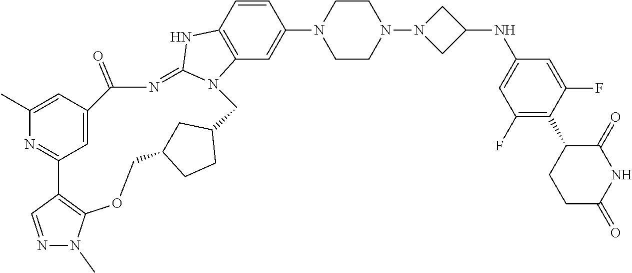

The racemate compound was prepared in a manner similar to that in Example 1 with intermediates 63 and 64.

Prep chiral-HPLC method:

Column: CHIRALPAK IE, 20*250 mm 5 μm

Mobile Phase A: MTBE

Mobile Phase B: MeOH:DCM=50:50 (0.2% FA+0.2% DEA)

Flow rate: 18 mL/min

Gradient: Mobile Phase A: Mobile Phase B=10:90 (v/v)

The title compound corresponds to peak A @ 11.3 min.

¹H NMR (500 MHz, DMSO) δ 12.41 (s, 1H), 10.84 (s, 1H), 8.52 (s, 1H), 7.89 (s, 1H), 7.44 (s, 1H), 7.34 (s, 1H), 7.01 (s, 1H), 6.91 (d, J=7.2 Hz, 1H), 6.68 (d, J=6.8 Hz, 1H), 6.20 (d, J=11.7 Hz, 2H), 4.33-3.93 (m, 7H), 3.74 (s, 3H), 3.66 (s, 2H), 3.56-3.52 (m, 2H), 2.85-2.73 (m, 6H), 2.63-2.53 (m, 5H), 2.19 (s, 1H), 2.10-2.00 (m, 1H), 1.93-1.65 (m, 8H), 1.39-1.33 (m, 2H). [M+H]⁺=819.6.

### Example 113: (R)-3-(4-((1′-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-[1,4′-bipiperidin]-4-yl)oxy)-2,6-difluorophenyl)piperidine-2,6-dione

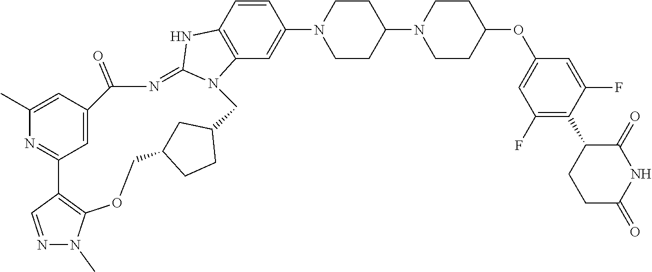

To a solution of intermediate 64 (60 mg, 0.11 mmol) and intermediate 65 (71 mg, 0.22 mmol) in DCE (6 mL) was added STAB (70 mg, 0.33 mmol). Then the mixture was stirred at 50° C. for 16 hours. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column chromatography (DCM:CH₃OH=15:1), followed by Prep-HPLC chromatography to afford the title product (15 mg, 16%).

¹H NMR (500 MHz, DMSO) δ 12.41 (s, 1H), 10.92 (s, 1H), 8.52 (s, 1H), 7.89 (s, 1H), 7.45 (s, 1H), 7.32 (d, J=8.7 Hz, 1H), 7.02 (s, 1H), 6.92 (d, J=9.0 Hz, 1H), 6.78 (d, J=10.9 Hz, 2H), 4.44 (d, J=6.3 Hz, 2H), 4.35-4.24 (m, 2H), 4.23-4.06 (m, 4H), 3.74 (s, 3H), 2.85-2.80 (m, 3H), 2.69 (t, J=11.4 Hz, 3H), 2.59 (s, 2H), 2.55 (s, 4H), 2.47-2.35 (m, 3H), 2.13-2.10 (m, 1H), 2.03-1.91 (m, 4H), 1.89-1.77 (m, 4H), 1.69-1.52 (m, 6H). [M+H]⁺=848.2.

### Example 114: 3-(5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-4-(hydroxymethyl)piperidin-1-yl)-6-methylpyridin-2-yl)piperidine-2,6-dione

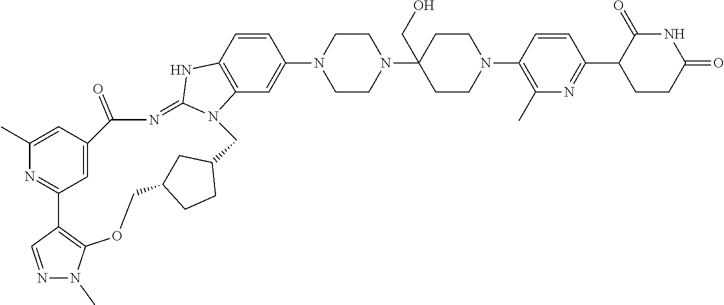

The title compound was prepared in a manner similar to that in Example 11 with intermediates 1 and 66.

¹H NMR (500 MHz, DMSO) δ 12.37 (s, 1H), 10.77 (s, 1H), 9.27 (s, 1H), 8.52 (s, 1H), 7.89 (s, 1H), 7.45 (s, 1H), 7.39 (d, J=8.3 Hz, 1H), 7.37 (d, J=8.8 Hz, 1H), 7.09 (d, J=8.2 Hz, 1H), 7.00 (s, 1H), 6.92 (d, J=8.7 Hz, 1H), 4.37-4.25 (m, 1H), 4.24-4.16 (m, 1H), 4.15-3.99 (m, 2H), 3.88 (dd, J=9.4, 5.3 Hz, 1H), 3.74 (s, 3H), 3.41 (s, 2H), 3.18-3.10 (m, 4H), 3.05-2.97 (m, 2H), 2.91-2.83 (m, 4H), 2.79-2.74 (m, 2H), 2.65-2.52 (m, 9H), 2.39 (s, 3H), 2.24-2.16 (m, 1H), 2.10-2.03 (m, 1H), 1.97-1.89 (m, 3H), 1.87-1.79 (m, 2H), 1.78-1.68 (m, 3H). [M+H]⁺=842.7.

### Example 115: 3-(4-((3S,4R)-4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)-3-fluoropiperidin-1-yl)-3-fluorophenoxy)piperidine-2,6-dione

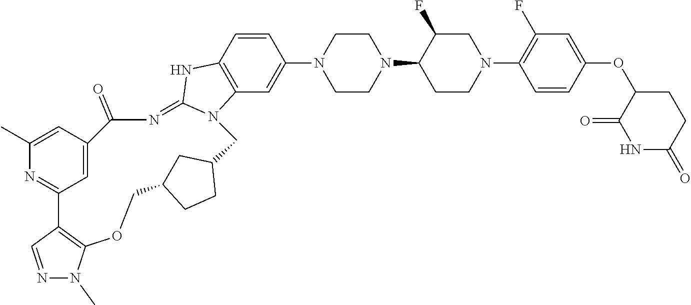

To a stirred solution of intermediate 1 (60 mg, 0.1 mmol in DMA (5 mL) were added intermediate 67 (47 mg, 0.1 mmol), t-BuONa (44 mg, 0.5 mmol), Ruphos (21 mg, 0.05 mmol) and Pd₂(dba)₃ (46 mg, 0.02 mmol) at room temperature under nitrogen atmosphere. The resulting mixture was stirred for 1 h at 50° C. under nitrogen atmosphere. The mixture was allowed to cool down to room temperature. The resulting mixture was concentrated under reduced pressure. The residue was diluted with DCM (50 mL), washed with water (3×20 mL) and brine (20 mL). The organic layer was dried over anhydrous Na₂SO₄, filtered and concentrated under reduced pressure. The residue was purified by prep-HPLC to afford the title compound (8.13 mg, 8.3%).

¹H NMR (500 MHz, DMSO) δ 12.42 (s, 1H), 10.93 (s, 1H), 8.52 (s, 1H), 7.89 (s, 1H), 7.45 (s, 1H), 7.37 (d, J=8.7 Hz, 1H), 7.04 (s, 1H), 6.99 (t, J=9.6 Hz, 1H), 6.93 (d, J=2.2 Hz, 1H), 6.92-6.89 (m, 1H), 6.78 (d, J=8.7 Hz, 1H), 5.15-5.12 (m, 2H), 4.31 (s, 1H), 4.19 (s, 1H), 4.09 (s, 2H), 3.74 (s, 3H), 3.52-3.48 (m, 1H), 3.37 (s, 1H), 3.19-3.18 (m, 4H), 2.93-2.76 (m, 5H), 2.74-2.56 (m, 6H), 2.55 (s, 3H), 2.21-2.17 (m, 1H), 2.16-1.96 (m, 3H), 1.91 (s, 1H), 1.82-1.79 (m, 4H), 1.68 (s, 1H). [M+H]⁺=849.7.

### Example 116: (R)-3-(4-((1′-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-[1,4′-bipiperidin]-4-yl)amino)-2,6-difluorophenyl)piperidine-2,6-dione

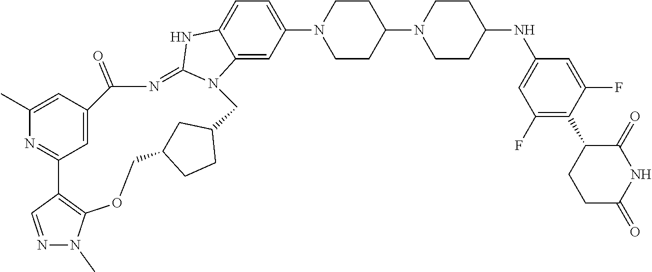

To a solution of intermediate 64 (80 mg, 0.15 mmol), intermediate 68 (59 mg, 0.18 mmol) and DIEA (58 mg, 0.45 mmol) in DCE (6 mL)/DMSO (2 mL) was added STAB (96 mg, 0.45 mmol). Then the mixture was stirred at 50° C. overnight. The reaction was quenched with sat. aq. NaHCO₃ (20 mL) and extracted with DCM (3×20 mL). The combined organic phase was washed with brine (1×15 mL), dried over Na₂SO₄, filtered and concentrated in vacuum. The residue was purified by silica gel column (DCM:CH₃OH=15:1) to give the racemate, which was further purified by Prep chiral-HPLC with following condition: (Column: CHIRALPAK IE, 20*250 mm 5 m; Mobile Phase A: MTBE (0.1% FA), Mobile Phase B: MeOH:DCM=50:50; Flow rate: 20 mL/min; Gradient: Mobile Phase A: Mobile Phase B=40:60 (v/v), Injection Volume: 5 uL; Wave Length: UV 254 nm and 220 nm; retention time: peak A 2.235 min, peak B 3.711 min) to give the peak A as the desired product (14 mg, 11.2%).

¹H NMR (500 MHz, DMSO) δ 12.42 (s, 1H), 10.83 (s, 1H), 8.52 (s, 1H), 7.89 (s, 1H), 7.45 (s, 1H), 7.36 (d, J=8.7 Hz, 1H), 7.03 (s, 1H), 6.93 (d, J=8.2 Hz, 1H), 6.25 (d, J=12.1 Hz, 2H), 6.15 (d, J=6.8 Hz, 1H), 4.35-4.03 (m, 4H), 3.97 (dd, J=12.5, 4.9 Hz, 1H), 3.79-3.72 (m, 5H), 3.26-3.22 (m, 2H), 2.97 (s, 2H), 2.80-2.54 (m, 11H), 2.10-1.78 (m, 11H), 1.64 (d, J=10.8 Hz, 3H), 1.46-1.35 (m, 2H). [M+H]⁺=847.6.

### Example 117: (R)-3-(4-(4-(6-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2,6-diazaspiro[3.3]heptan-2-yl)piperidin-1-yl)-2,6-difluorophenyl)piperidine-2,6-dione

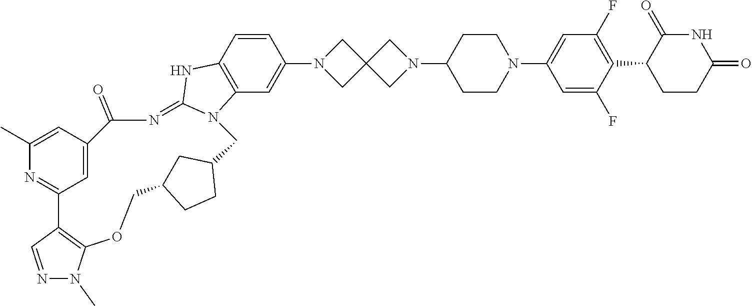

The title compound was prepared in a manner similar to that in Example 1 with intermediates 69 and 7.

¹H NMR (500 MHz, DMSO) δ 12.39 (s, 1H), 10.87 (s, 1H), 8.51 (s, 1H), 7.89 (s, 1H), 7.44 (s, 1H), 7.34 (d, J=8.4 Hz, 1H), 6.63 (d, J=12.3 Hz, 2H), 6.49 (s, 1H), 6.37 (d, J=8.3 Hz, 1H), 4.34-3.87 (m, 10H), 3.77-3.60 (m, 6H), 2.88-2.74 (m, 4H), 2.65-2.51 (m, 8H), 2.22-2.03 (m, 2H), 1.99-1.60 (m, 10H). [M+H]⁺=845.6.

### Example 118: 3-((4-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-3-methylphenyl)amino)piperidine-2,6-dione

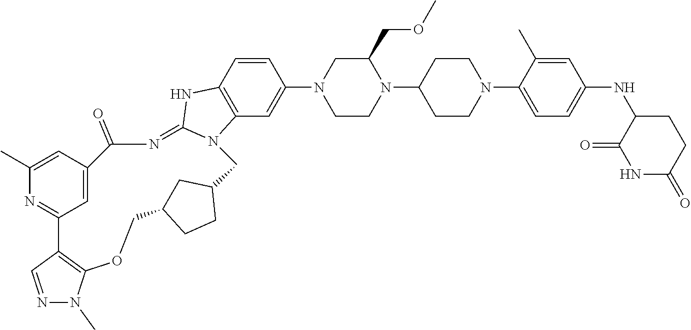

The title compound was prepared in a manner similar to that in Example 1 with intermediates 3 and 70.

¹H NMR (500 MHz, DMSO) δ 12.42 (s, 1H), 10.75 (s, 1H), 8.52 (s, 1H), 7.89 (s, 1H), 7.45 (s, 1H), 7.37 (d, J=8.7 Hz, 1H), 6.99 (s, 1H), 6.89 (d, J=8.7 Hz, 1H), 6.83 (d, J=8.5 Hz, 1H), 6.52 (s, 1H), 6.46 (d, J=8.4 Hz, 1H), 5.44 (d, J=7.3 Hz, 1H), 4.38-4.27 (m, 1H), 4.26-4.17 (m, 2H), 4.14-4.03 (m, 2H), 3.74 (s, 3H), 3.57-3.54 (m, 2H), 3.27-3.21 (m, 4H), 3.05 (t, J=7.4 Hz, 2H), 2.98-2.96 (m, 3H), 2.89 (m, 2H), 2.84-2.68 (m, 4H), 2.62-2.58 (m, 6H), 2.55 (s, 3H), 2.16 (s, 3H), 2.10-2.08 (m, 1H), 1.97-1.80 (m, 6H), 1.74 (d, J=9.0 Hz, 2H), 1.60-1.49 (m, 1H). [M+H]⁺=870.2.

### Example 119: (R)-3-(4-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-methylpiperazin-1-yl)piperidin-1-yl)-2,6-difluorophenyl)piperidine-2,6-dione

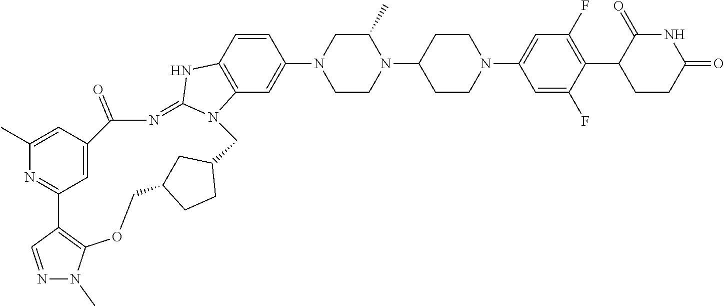

The title compound was prepared in a manner similar to that in Example 1 with intermediates 71 and 7.

¹H NMR (500 MHz, DMSO) δ 12.41 (s, 1H), 10.87 (s, 1H), 8.52 (s, 1H), 7.89 (s, 1H), 7.45 (s, 1H), 7.36 (d, J=8.7 Hz, 1H), 6.99 (s, 1H), 6.90 (d, J=8.9 Hz, 1H), 6.65 (s, 1H), 6.63 (s, 1H), 4.29 (s, 1H), 4.19 (s, 1H), 4.11-4.02 (m, 3H), 3.84 (d, J=11.9 Hz, 2H), 3.74 (s, 3H), 3.43-3.37 (m, 2H), 3.30 (s, 2H), 2.97-2.93 (m, 2H), 2.89-2.82 (m, 3H), 2.81-2.72 (m, 3H), 2.64 (t, J=9.8 Hz, 2H), 2.55 (s, 4H), 2.12-2.07 (m, 1H), 1.99-1.90 (m, 2H), 1.88-1.74 (m, 4H), 1.68-1.65 (m, 3H), 1.43 (d, J=10.0 Hz, 1H), 1.13 (d, J=6.1 Hz, 3H); [M+H]⁺=847.5

### Example 120: (R)-3-(4-(4-(7-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2,7-diazaspiro[3.5]nonan-2-yl)piperidin-1-yl)-2,6-difluorophenyl)piperidine-2,6-dione

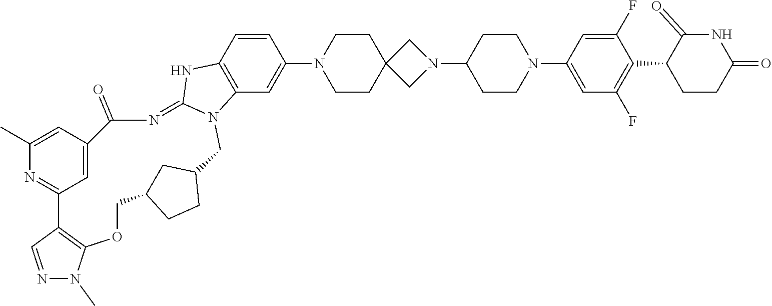

The title compound was prepared in a manner similar to that in Example 1 with intermediates 72 and 7.

¹H NMR (500 MHz, DMSO) δ 12.41 (s, 1H), 10.87 (s, 1H), 8.52 (s, 1H), 7.89 (s, 1H), 7.44 (s, 1H), 7.35 (d, J=8.7 Hz, 1H), 7.02 (s, 1H), 6.91 (d, J=8.8 Hz, 1H), 6.63 (s, 1H), 6.60 (s, 1H), 4.29 (s, 1H), 4.18 (d, J=7.9 Hz, 1H), 4.09 (s, 1H), 4.05 (d, J=4.9 Hz, 1H), 4.03 (d, J=4.9 Hz, 1H), 3.74 (s, 3H), 3.63 (d, J=12.5 Hz, 2H), 3.10 (s, 4H), 2.98 (s, 4H), 2.84 (t, J=10.7 Hz, 2H), 2.79-2.74 (m, 1H), 2.64-2.57 (m, 2H), 2.55 (s, 3H), 2.52-2.47 (m, 3H), 2.25 (s, 1H), 2.12-2.04 (m, 1H), 1.97-1.91 (m, 2H), 1.81 (s, 6H), 1.69 (d, J=10.3 Hz, 3H), 1.23-1.19 (m, 2H); [M+H]⁺=873.5

### Example 121: (R)-3-(2,6-difluoro-4-(4-(3-((S)-2-(methoxymethyl)-4-((7R,7³S,E)-1¹,1³ 2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)azetidin-1-yl)piperidin-1-yl)phenyl)piperidine-2,6-dione

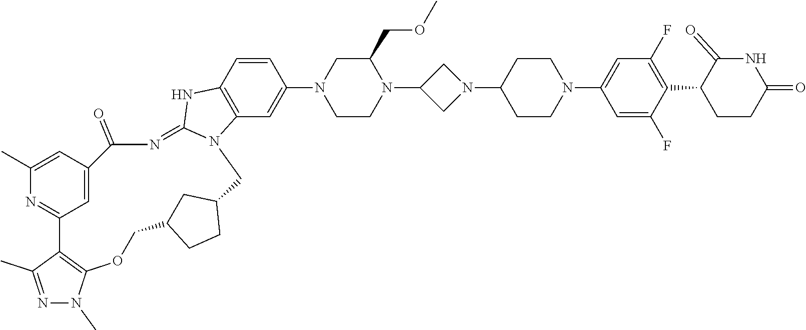

The title compound was prepared in a manner similar to that in Example 1 with intermediates 73 and 7.

¹H NMR (500 MHz, DMSO) δ 12.40 (s, 1H), 10.87 (s, 1H), 8.54 (s, 1H), 7.45 (s, 1H), 7.37 (d, J=8.7 Hz, 1H), 6.99 (s, 1H), 6.87 (dd, J=9.0, 1.0 Hz, 1H), 6.61 (d, J=12.8 Hz, 2H), 4.38-3.98 (m, 5H), 3.67 (s, 3H), 3.62-3.52 (m, 2H), 3.50-3.38 (m, 8H), 3.28-3.18 (m, 7H), 3.11-2.99 (m, 2H), 2.90-2.73 (m, 6H), 2.70-2.51 (m, 8H), 2.38-2.33 (m, 1H), 2.22-2.15 (m, 1H), 2.13-2.04 (m, 1H), 1.97-1.92 (m, 1H), 1.89-1.77 (m, 3H), 1.72-1.65 (m, 2H), 1.27-1.17 (m, 2H). [M+H]⁺=946.2.

### Example 122: (R)-3-(2,6-difluoro-4-(4-(4-((7¹R,7³S,E)-1¹-(2-hydroxyethyl)-2⁶-methyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

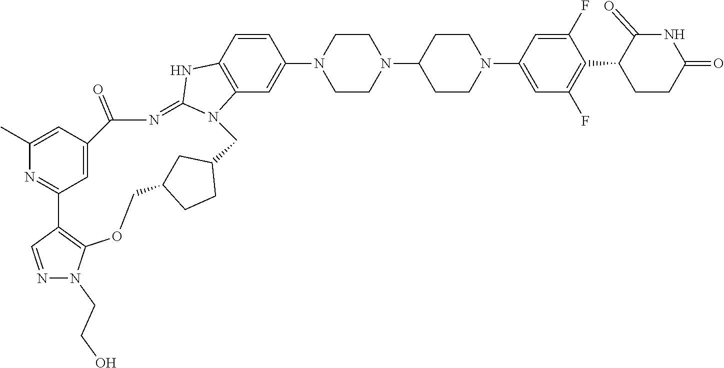

To a solution of intermediate 76 (90 mg, 0.16 mmol) and intermediate 7 (73 mg, 0.23 mmol) in DCE (6 mL) was added STAB (102 mg, 0.48 mmol). Then the mixture was stirred at 50° C. overnight. The reaction was quenched with sat. aq. NaHCO₃ (15 mL) and extracted with DCM (3×15 mL). The combined organic phase was washed with brine (1×10 mL), dried over Na₂SO₄, filtered and concentrated in vacuum. The residue was purified by silica gel column (DCM:CH₃OH=10:1), followed by Prep-HPLC chromatography to afford the title product (9.0 mg, 6.5%).

¹H NMR (500 MHz, DMSO) δ 12.41 (s, 1H), 10.87 (s, 1H), 8.50 (s, 1H), 7.94 (s, 1H), 7.44 (s, 1H), 7.36 (d, J=8.7 Hz, 1H), 7.01 (s, 1H), 6.91 (d, J=8.9 Hz, 1H), 6.64 (d, J=12.7 Hz, 2H), 4.97 (t, J=5.4 Hz, 1H), 4.34-4.01 (m, 7H), 3.86-3.78 (m, 4H), 3.19-3.13 (m, 4H), 2.81-2.74 (m, 3H), 2.71-2.54 (m, 11H), 2.45-2.35 (m, 1H), 2.14-2.03 (m, 1H), 1.99-1.66 (m, 8H), 1.49 (d, J=8.9 Hz, 2H). [M+H]⁺=863.6.

### Example 123: 3-((4-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-methylphenyl)amino)piperidine-2,6-dione

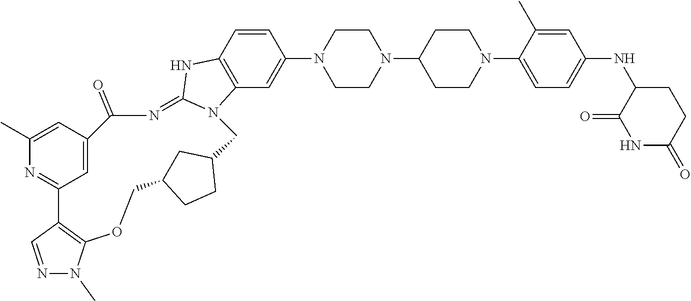

The title compound was prepared in a manner similar to that in Example 1 with intermediates 2 and 70.

¹H NMR (500 MHz, DMSO) δ 12.42 (s, 1H), 10.75 (s, 1H), 8.52 (s, 1H), 7.89 (s, 1H), 7.45 (s, 1H), 7.37 (d, J=8.7 Hz, 1H), 7.03 (s, 1H), 6.92 (d, J=8.8 Hz, 1H), 6.83 (d, J=8.7 Hz, 1H), 6.52 (d, J=2.3 Hz, 1H), 6.46 (d, J=8.6 Hz, 1H), 5.44 (d, J=7.4 Hz, 1H), 4.30 (s, 1H), 4.25-4.17 (m, 2H), 4.10 (s, 2H), 3.74 (s, 3H), 3.18 (s, 4H), 2.95 (s, 2H), 2.71 (d, J=5.2 Hz, 5H), 2.59 (d, J=4.8 Hz, 3H), 2.55 (s, 5H), 2.38-2.31 (m, 1H), 2.16 (s, 3H), 2.13-2.06 (m, 1H), 1.94-1.82 (m, 7H), 1.60-1.50 (m, 4H). [M+H]⁺=826.2.

### Example 124: (R)-3-(4-(4-((1-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)azetidin-3-yl)(methyl)amino)piperidin-1-yl)-2,6-difluorophenyl)piperidine-2,6-dione

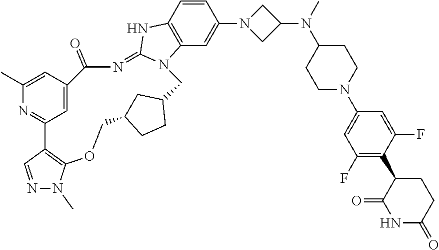

The title compound was prepared in a manner similar to that in Example 1 with intermediates 77 and 7.

¹H NMR (500 MHz, DMSO) δ 12.37 (s, 1H), 10.87 (s, 1H), 8.52 (s, 1H), 7.89 (s, 1H), 7.44 (s, 1H), 7.33 (d, J=8.5 Hz, 1H), 6.64 (d, J=12.7 Hz, 2H), 6.52 (d, J=1.7 Hz, 1H), 6.38 (dd, J=8.6, 1.9 Hz, 1H), 4.34-4.24 (m, 1H), 4.19 (s, 1H), 4.10 (d, J=8.7 Hz, 1H), 4.07-4.01 (m, 4H), 3.88-3.84 (m, 2H), 3.77-3.73 (m, 4H), 3.60 (t, J=6.0 Hz, 2H), 2.84-2.67 (m, 4H), 2.65-2.57 (m, 4H), 2.54 (s, 4H), 2.15 (s, 3H), 2.10-2.02 (m, 1H), 2.00-1.89 (m, 2H), 1.88-1.84 (m, 2H), 1.72-1.62 (m, 3H), 1.57-1.46 (m, 2H); [M+H]⁺=833.7.

### Example 125: (R)-3-(4-(4-(2-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2,7-diazaspiro[3.5]nonan-7-yl)piperidin-1-yl)-2,6-difluorophenyl)piperidine-2,6-dione

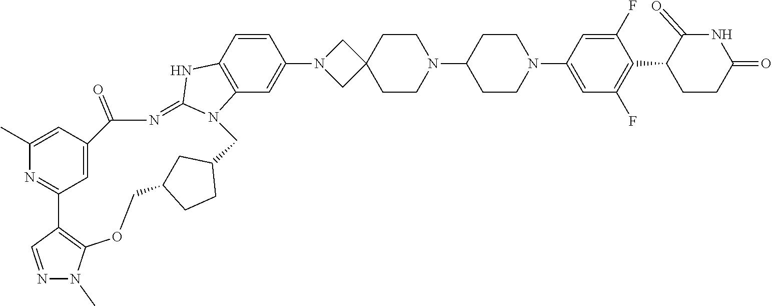

The title compound was prepared in a manner similar to that in Example 1 with intermediates 78 and 7.

¹H NMR (500 MHz, DMSO) δ 12.36 (s, 1H), 10.87 (s, 1H), 8.51 (s, 1H), 7.89 (s, 1H), 7.44 (s, 1H), 7.32 (d, J=8.5 Hz, 1H), 6.62 (d, J=12.8 Hz, 2H), 6.48 (s, 1H), 6.35 (d, J=8.6 Hz, 1H), 4.35-4.22 (m, 1H), 4.19 (t, J=7.8 Hz, 1H), 4.11 (dd, J=10.4, 2.7 Hz, 1H), 4.04 (dt, J=12.7, 4.6 Hz, 1H), 3.99 (d, J=5.6 Hz, 1H), 3.80 (d, J=12.1 Hz, 2H), 3.74 (s, 3H), 3.57 (s, 4H), 2.83-2.68 (m, 4H), 2.65-2.52 (m, 7H), 2.43-2.41 (m, 4H), 2.14-2.02 (m, 1H), 1.98-1.88 (m, 2H), 1.84-1.81 (m, 2H), 1.79-1.70 (m, 7H), 1.69-1.61 (m, 1H), 1.52-1.42 (m, 2H). [M+H]⁺=873.7.

### Example 126: (R)-3-(4-(3-(2-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2,7-diazaspiro[3.5]nonan-7-yl)azetidin-1-yl)-2,6-difluorophenyl)piperidine-2,6-dione

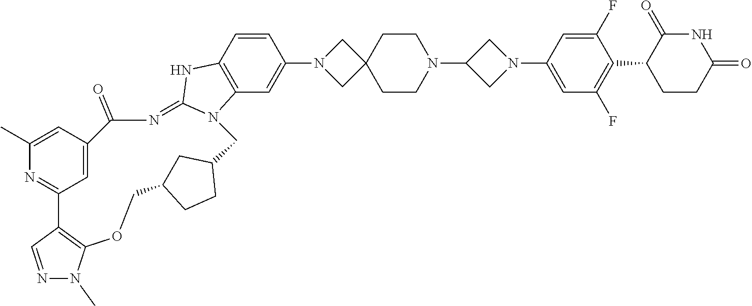

The title compound was prepared in a manner similar to that in Example 1 with intermediates 78 and 79.

¹H NMR (500 MHz, DMSO) δ 12.35 (s, 1H), 10.86 (s, 1H), 8.51 (s, 1H), 7.89 (s, 1H), 7.44 (s, 1H), 7.33 (d, J=8.5 Hz, 1H), 6.48 (s, 1H), 6.36 (dd, J=8.6, 1.7 Hz, 1H), 6.13 (d, J=11.1 Hz, 2H), 4.35-4.24 (m, 1H), 4.19 (dd, J=13.9, 7.0 Hz, 1H), 4.12-4.10 (m, 1H), 4.03 (dd, J=12.3, 4.9 Hz, 2H), 3.93 (t, J=7.3 Hz, 2H), 3.74 (s, 3H), 3.66-3.61 (m, 2H), 3.59 (s, 4H), 3.29 (s, 1H), 3.22 (d, J=6.0 Hz, 1H), 2.82-2.72 (m, 1H), 2.66-2.52 (m, 7H), 2.36-2.19 (m, 3H), 2.13-2.02 (m, 1H), 1.95-1.91 (m, 2H), 1.87-1.72 (m, 7H), 1.68-1.64 (m, 1H); [M+H]⁺=845.7.

### Example 127: (R)-3-(2,6-difluoro-4-(4-(((S)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)methyl)piperidin-1-yl)phenyl)piperidine-2,6-dione

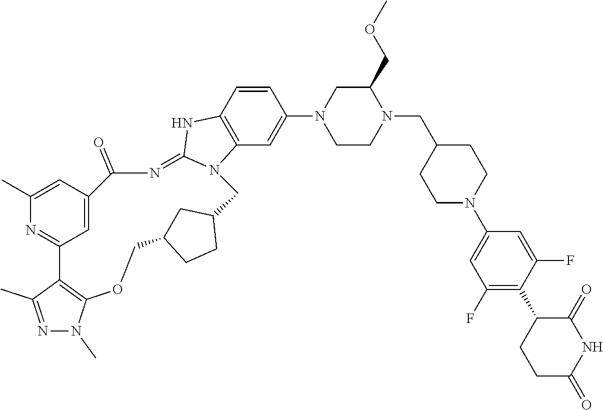

The title compound was prepared in a manner similar to that in Example 1 with intermediates 40 and 80.

¹H NMR (500 MHz, DMSO) δ 12.44 (s, 1H), 10.87 (s, 1H), 8.54 (s, 1H), 7.45 (s, 1H), 7.37 (d, J=8.7 Hz, 1H), 7.01 (s, 1H), 6.89 (d, J=8.7 Hz, 1H), 6.63 (s, 1H), 6.60 (s, 1H), 4.39-3.95 (m, 5H), 3.76 (d, J=12.3 Hz, 2H), 3.67 (s, 3H), 3.55 (dd, J=9.5, 4.1 Hz, 1H), 3.44 (dd, J=9.5, 6.2 Hz, 1H), 3.40-3.32 (m, 2H), 3.29-3.22 (m, 4H), 3.01 (t, J=8.4 Hz, 1H), 2.96-2.91 (m, 1H), 2.90-2.83 (m, 1H), 2.83-2.70 (m, 3H), 2.69-2.56 (m, 4H), 2.55 (s, 3H), 2.51 (s, 1H), 2.49-2.45 (m, 4H), 2.45-2.38 (m, 1H), 2.20 (dd, J=12.5, 5.1 Hz, 1H), 2.14-2.03 (m, 1H), 2.00-1.93 (m, 1H), 1.90-1.77 (m, 4H), 1.77-1.61 (m, 3H), 1.21-1.08 (m, 2H). [M+H]⁺=905.7

### Example 129: (R)-3-(2,6-difluoro-4-((1′-((7¹R,7³S,E)-1¹-(2-hydroxyethyl)-2⁶-methyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-[1,4′-bipiperidin]-4-yl)amino)phenyl)piperidine-2,6-dione

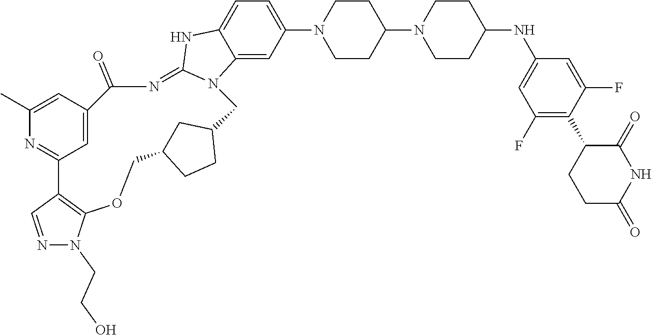

The title compound was prepared in a manner similar to that in Example 1 with intermediates 140 and 68.

¹H NMR (500 MHz, DMSO) δ 12.40 (s, 1H), 10.82 (s, 1H), 8.50 (s, 1H), 7.94 (s, 1H), 7.44 (s, 1H), 7.35 (d, J=8.7 Hz, 1H), 7.02 (s, 1H), 6.92 (d, J=9.1 Hz, 1H), 6.23 (d, J=12.1 Hz, 2H), 6.12 (d, J=8.0 Hz, 1H), 4.97 (t, J=5.4 Hz, 1H), 4.28-3.95 (m, 8H), 3.85-3.71 (m, 5H), 3.19 (s, 2H), 2.87-2.58 (m, 10H), 2.41-2.29 (m, 5H), 2.05 (t, J=12.6 Hz, 1H), 1.93-1.84 (m, 7H), 1.60 (d, J=12.3 Hz, 2H), 1.34 (d, J=10.5 Hz, 2H). [M+H]⁺=877.6.

### Example 131: (R)-3-(4-((1-((1-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)methyl)piperidin-4-yl)amino)-2,6-difluorophenyl)piperidine-2,6-dione

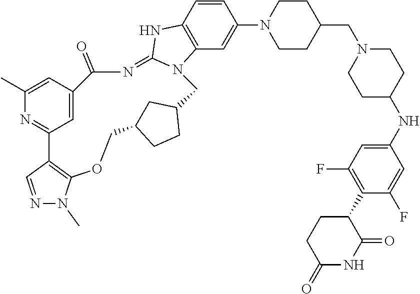

The title compound was prepared in a manner similar to that in Example 1 with intermediates 82 and 68.

¹H NMR (500 MHz, DMSO) δ 12.42 (s, 1H), 10.83 (s, 1H), 8.52 (s, 1H), 7.89 (s, 1H), 7.45 (s, 1H), 7.36 (d, J=8.7 Hz, 1H), 7.01 (s, 1H), 6.93 (d, J=8.6 Hz, 1H), 6.29 (d, J=11.4 Hz, 3H), 4.35-4.28 (m, 1H), 4.22-4.17 (m, 1H), 4.12-4.06 (m, 2H), 4.00-3.97 (m, 1H), 3.74 (s, 3H), 3.72-3.68 (m, 3H), 3.10-2.97 (m, 2H), 2.82-2.67 (m, 4H), 2.64-2.58 (m, 3H), 2.55 (s, 3H), 2.51-2.45 (m, 3H), 2.11-2.02 (m, 3H), 1.94-1.82 (m, 8H), 1.68 (s, 2H), 1.40-1.32 (m, 2H), 1.23-1.19 (m, 2H). [M+H]⁺=861.5.

### Example 132: (R)-3-(4-((1-((1-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)methyl)azetidin-3-yl)amino)-2,6-difluorophenyl)piperidine-2,6-dione

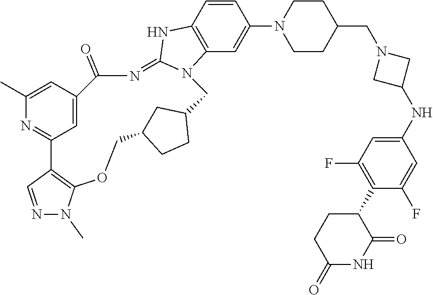

The title compound was prepared in a manner similar to that in Example 1 with intermediates 82 and 63.

¹H NMR (500 MHz, DMSO) δ 12.41 (s, 1H), 10.84 (s, 1H), 8.52 (s, 1H), 7.89 (s, 1H), 7.44 (s, 1H), 7.35 (d, J=8.7 Hz, 1H), 7.00 (s, 1H), 6.90 (d, J=8.8 Hz, 1H), 6.70 (d, J=6.6 Hz, 1H), 6.20 (s, 1H), 6.18 (s, 1H), 4.36-4.24 (m, 1H), 4.19 (t, J=7.8 Hz, 1H), 4.14-3.93 (m, 4H), 3.81-3.59 (m, 7H), 3.43-3.34 (m, 2H), 2.90-2.53 (m, 10H), 2.48-2.38 (m, 2H), 2.10-2.01 (m, 1H), 1.96-1.89 (m, 2H), 1.87-1.75 (m, 4H), 1.69-1.62 (m, 1H), 1.49-1.41 (m, 1H), 1.33-1.20 (m, 3H). [M+H]⁺=833.7

### Example 133: 3-((3,5-difluoro-4-(4-((S)-2-(methoxymethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)amino)piperidine-2,6-dione

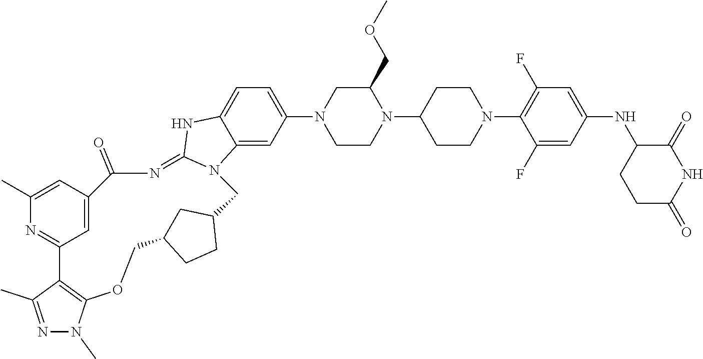

The title compound was prepared in a manner similar to that in Example 1 with intermediates 40 and 83.

¹H NMR (500 MHz, DMSO) δ 12.43 (s, 1H), 10.81 (s, 1H), 8.54 (s, 1H), 7.45 (s, 1H), 7.37 (d, J=8.7 Hz, 1H), 6.99 (s, 1H), 6.88 (d, J=8.7 Hz, 1H), 6.33 (s, 1H), 6.31 (s, 1H), 6.23 (d, J=7.9 Hz, 1H), 4.41-3.95 (m, 5H), 3.67 (s, 3H), 3.58-3.54 (m, 1H), 3.50-3.45 (m, 1H), 3.40-3.33 (m, 2H), 3.30 (s, 3H), 3.30-3.26 (m, 1H), 3.07-2.86 (m, 8H), 2.83-2.53 (m, 10H), 2.48 (s, 3H), 2.11-2.03 (m, 1H), 1.92-1.63 (m, 8H), 1.55-1.44 (m, 1H). [M+H]⁺=906.7

### Example 134: (R)-3-(2,6-difluoro-4-(4-(((R)-2-methyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)piperidin-1-yl)phenyl)piperidine-2,6-dione

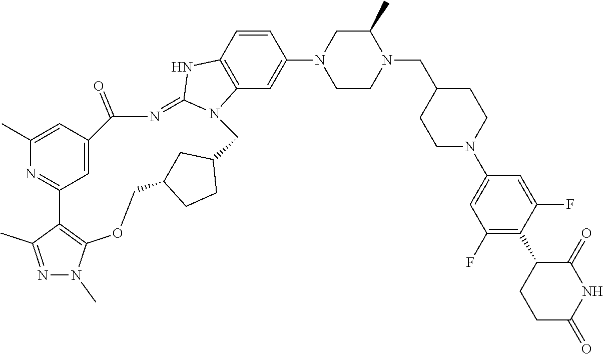

The title compound was prepared in a manner similar to that in Example 1 with intermediates 84 and 80.

¹H NMR (500 MHz, DMSO) δ 12.43 (s, 1H), 10.87 (s, 1H), 8.54 (s, 1H), 7.45 (s, 1H), 7.36 (d, J=8.7 Hz, 1H), 7.02 (s, 1H), 6.90 (d, J=8.1 Hz, 1H), 6.63 (s, 1H), 6.60 (s, 1H), 4.40-3.93 (m, 5H), 3.80-3.72 (m, 2H), 3.67 (s, 3H), 3.49-3.38 (m, 2H), 2.98 (d, J=11.0 Hz, 1H), 2.88 (t, J=9.7 Hz, 1H), 2.83-2.70 (m, 3H), 2.65-2.51 (m, 8H), 2.49-2.43 (m, 4H), 2.30 (t, J=9.4 Hz, 1H), 2.14-1.60 (m, 11H), 1.27-1.09 (m, 3H), 1.07 (d, J=5.9 Hz, 3H). [M+H]⁺=875.7

### Example 135: (R)-3-(2,6-difluoro-4-(4-((S)-2-methyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

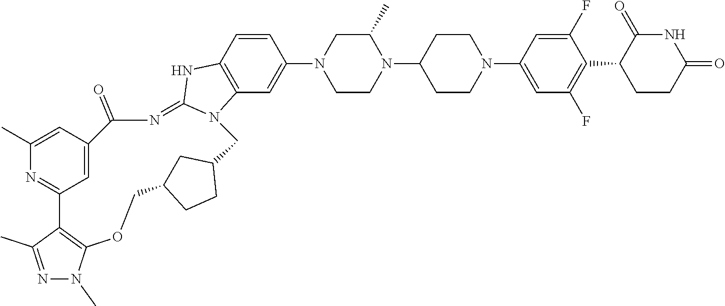

The title compound was prepared in a manner similar to that in Example 1 with intermediates 85 and 7.

¹H NMR (500 MHz, DMSO) δ 12.43 (s, 1H), 10.87 (s, 1H), 8.54 (s, 1H), 7.45 (s, 1H), 7.37 (d, J=8.4 Hz, 1H), 7.01 (s, 1H), 6.89 (d, J=8.6 Hz, 1H), 6.64 (d, J=12.8 Hz, 2H), 4.37-4.24 (m, 1H), 4.22-4.15 (m, 1H), 4.13-3.93 (m, 3H), 3.91-3.85 (m, 1H), 3.81-3.78 (m, 2H), 3.66 (s, 3H), 3.22-3.20 (m, 1H), 3.03-2.99 (m, 1H), 2.89-2.73 (m, 4H), 2.72-2.51 (m, 9H), 2.49-2.34 (m, 6H), 2.11-2.07 (m, 1H), 2.00-1.93 (m, 1H), 1.85-1.64 (m, 6H), 1.54-1.47 (m, 2H), 0.96-0.95 (m, 3H); [M+H]⁺=861.7.

### Example 136: (R)-3-(2,6-difluoro-4-(4-(methyl(1-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)amino)piperidin-1-yl)phenyl)piperidine-2,6-dione

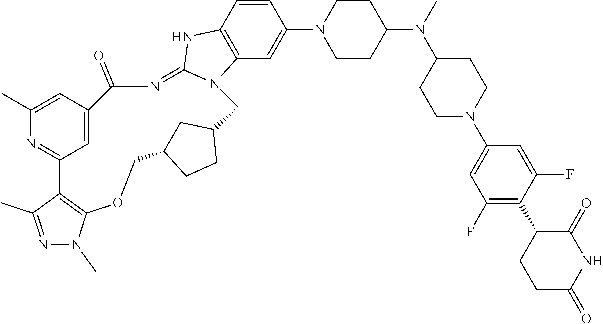

The title compound was prepared in a manner similar to that in Example 1 with intermediates 86 and 7.

¹H NMR (500 MHz, DMSO) δ 12.41 (s, 1H), 10.86 (s, 1H), 8.53 (s, 1H), 7.45 (s, 1H), 7.35 (d, J=8.7 Hz, 1H), 7.01 (s, 1H), 6.90 (dd, J=8.9, 1.9 Hz, 1H), 6.62 (d, J=12.8 Hz, 2H), 4.36-4.25 (m, 1H), 4.22 (s, 1H), 4.13-3.96 (m, 3H), 3.81 (d, J=13.1 Hz, 2H), 3.67 (s, 5H), 2.82-2.62 (m, 7H), 2.55 (s, 4H), 2.48 (s, 4H), 2.21 (s, 3H), 2.09-2.06 (m, 1H), 1.96-1.93 (m, 1H), 1.90-1.76 (m, 5H), 1.76-1.73 (m, 2H), 1.68-1.53 (m, 2H), 1.51-1.40 (m, 2H), 1.33-1.18 (m, 4H). [M+H]⁺=875.7.

### Example 137: 3-((5-(4-(4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-6-methylpyridin-2-yl)oxy)piperidine-2,6-dione

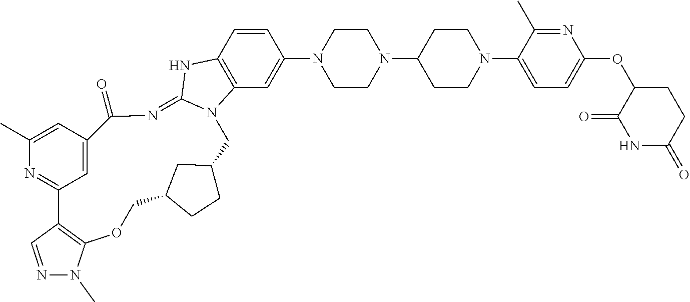 [SMILES: Cc1cc2cc(n1)-c1cnn(C)c1OC[C@H]1CC[C@H](C1)CN1/C(=N/C2=O)Nc2ccc(N3CCN(C4CCN(c5ccc(OC6CCC(=O)NC6=O)nc5C)CC4)CC3)cc21]

The title compound was prepared in a manner similar to that in Example 1 with intermediates 2 and 87.

¹H NMR (500 MHz, DMSO) δ 12.37 (s, 1H), 10.87 (s, 1H), 8.52 (s, 1H), 7.89 (s, 1H), 7.48 (d, J=8.7 Hz, 1H), 7.45 (s, 1H), 7.37 (d, J=8.7 Hz, 1H), 7.03 (s, 1H), 6.92 (d, J=8.7 Hz, 1H), 6.66 (d, J=8.5 Hz, 1H), 5.87-5.76 (m, 1H), 4.37-4.24 (m, 1H), 4.19 (s, 1H), 4.10 (dd, J=8.1, 5.6 Hz, 2H), 3.74 (s, 3H), 3.17 (t, J=7.7 Hz, 4H), 3.03-3.01 (m, 2H), 2.85-2.81 (m, 1H), 2.75-2.71 (m, 4H), 2.64-2.61 (m, 6H), 2.55 (s, 3H), 2.40-2.34 (m, 4H), 2.19-2.16 (m, 2H), 2.00-1.76 (m, 6H), 1.66-1.62 (m, 3H). [M+H]⁺=828.7.

### Example 138: (R)-3-(2,6-difluoro-4-(4-(methyl((1-((7¹R,7³S,E)-1¹,1³,2⁶-trimethyl-3-oxo-5²′5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)methyl)amino)piperidin-1-yl)phenyl)piperidine-2,6-dione

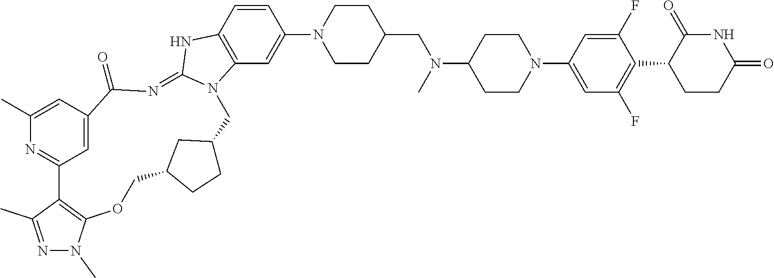 [SMILES: Cc1cc2cc(n1)-c1c(C)nn(C)c1OC[C@H]1CC[C@H](C1)CN1/C(=N/C2=O)Nc2ccc(N3CCC(CN(C)C4CCN(c5cc(F)c([C@H]6CCC(=O)NC6=O)c(F)c5)CC4)CC3)cc21]

The title compound was prepared in a manner similar to that in example 1 with intermediates 88 and 7.

¹H NMR (500 MHz, DMSO) δ 12.41 (s, 1H), 10.86 (s, 1H), 8.53 (s, 1H), 7.45 (s, 1H), 7.35 (d, J=8.7 Hz, 1H), 7.01 (s, 1H), 6.90 (dd, J=8.9, 1.9 Hz, 1H), 6.62 (d, J=12.8 Hz, 2H), 4.36-4.25 (m, 1H), 4.22 (s, 1H), 4.13-3.96 (m, 3H), 3.81 (d, J=13.1 Hz, 2H), 3.67 (s, 5H), 2.82-2.62 (m, 7H), 2.55 (s, 4H), 2.48 (s, 4H), 2.29 (d, J=6.5 Hz, 2H), 2.21 (s, 3H), 2.09-2.06 (m, 1H), 1.96-1.93 (m, 1H), 1.90-1.76 (m, 5H), 1.76-1.73 (m, 2H), 1.68-1.53 (m, 2H), 1.51-1.40 (m, 2H), 1.33-1.18 (m, 4H); [M+H]⁺=889.5.

### Example 140: 3-(4-(4-((R)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-methylphenoxy)piperidine-2,6-dione

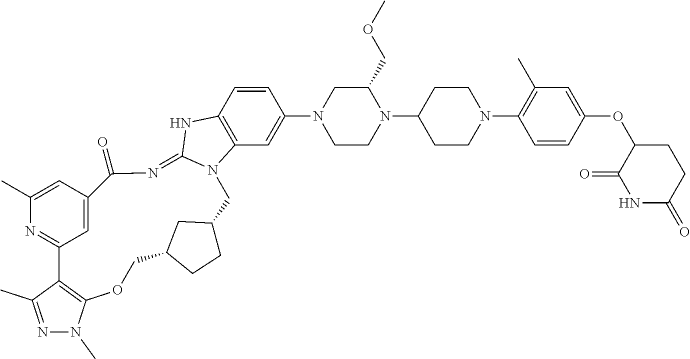 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2ccc(OC3CCC(=O)NC3=O)cc2C)CC1]

The title compound was prepared in a manner similar to that in example 1 with intermediates 47 and 89.

¹H NMR (500 MHz, DMSO) δ 12.42 (s, 1H), 10.89 (s, 1H), 8.54 (s, 1H), 7.45 (s, 1H), 7.37 (d, J=8.7 Hz, 1H), 7.02-6.94 (m, 2H), 6.88 (d, J=8.2 Hz, 1H), 6.84 (d, J=2.8 Hz, 1H), 6.79 (dd, J=8.7, 2.8 Hz, 1H), 5.07 (dd, J=10.6, 5.0 Hz, 1H), 4.35-3.96 (m, 4H), 3.67 (s, 3H), 3.58-3.49 (m, 2H), 3.31-3.22 (m, 4H), 3.09-2.95 (m, 5H), 2.91-2.78 (m, 2H), 2.75-2.52 (m, 12H), 2.48 (s, 3H), 2.22 (s, 3H), 2.19-2.07 (m, 2H), 1.92-1.71 (m, 7H), 1.66-1.50 (m, 2H). [M+H]⁺=885.6.

### Example 141: 3-(5-(4-((R)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4,6-dimethylpyridin-2-yl)piperidine-2,6-dione

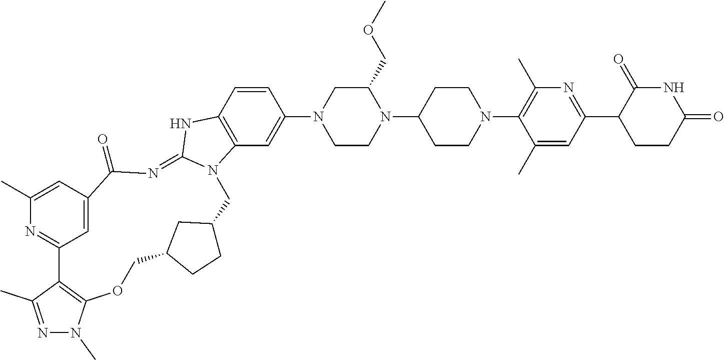 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(C3CCC(=O)NC3=O)nc2C)CC1]

The title compound was prepared in a manner similar to that in example 1 with intermediates 47 and 13.

¹H NMR (500 MHz, DMSO) δ 12.42 (s, 1H), 10.78 (s, 1H), 8.54 (s, 1H), 7.45 (s, 1H), 7.38 (d, J=8.7 Hz, 1H), 7.03-6.84 (m, 3H), 4.38-3.95 (m, 4H), 3.84 (dd, J=9.1, 5.3 Hz, 1H), 3.67 (s, 3H), 3.59 (dd, J=9.7, 4.3 Hz, 1H), 3.51 (dd, J=9.5, 6.0 Hz, 1H), 3.28-2.79 (m, 13H), 2.74-2.52 (m, 10H), 2.48 (s, 3H), 2.41 (d, J=6.9 Hz, 3H), 2.28 (s, 3H), 2.22-2.15 (m, 1H), 2.07 (dd, J=13.2, 5.6 Hz, 1H), 1.94-1.49 (m, 9H). [M+H]⁺=884.6.

### Example 142: (R)-3-(2,6-difluoro-4-(3-(methyl(1-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)amino)azetidin-1-yl)phenyl)piperidine-2,6-dione

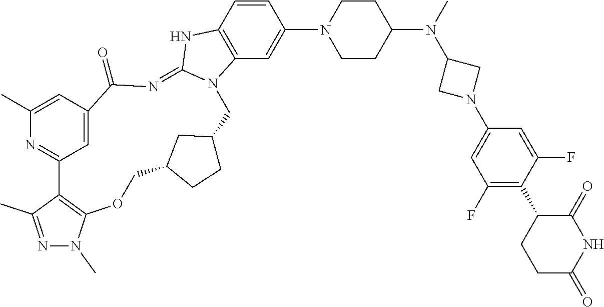 [SMILES: Cc1cc2cc(n1)-c1c(C)nn(C)c1OC[C@H]1CC[C@H](C1)CN1/C(=N/C2=O)Nc2ccc(N3CCC(N(C)C4CN(c5cc(F)c([C@H]6CCC(=O)NC6=O)c(F)c5)C4)CC3)cc21]

The title compound was prepared in a manner similar to that in example 1 with intermediates 86 and 79.

¹H NMR (500 MHz, DMSO) δ 12.41 (s, 1H), 10.86 (s, 1H), 8.53 (s, 1H), 7.45 (s, 1H), 7.35 (s, 1H), 7.03 (s, 1H), 6.91 (d, J=8.7 Hz, 1H), 6.14 (d, J=11.1 Hz, 2H), 4.36-3.99 (m, 5H), 3.95 (s, 2H), 3.84-3.71 (m, 3H), 3.68-3.62 (m, 5H), 2.83-2.73 (m, 1H), 2.67 (t, J=11.6 Hz, 2H), 2.61-2.52 (m, 8H), 2.48 (s, 3H), 2.19 (s, 3H), 2.12-2.03 (m, 1H), 1.97-1.58 (m, 10H). [M+H]⁺=847.6.

### Example 143: (R)-3-(2,6-difluoro-4-(4-(methyl((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)amino)-[1,4′-bipiperidin]-1-yl)phenyl)piperidine-2,6-dione

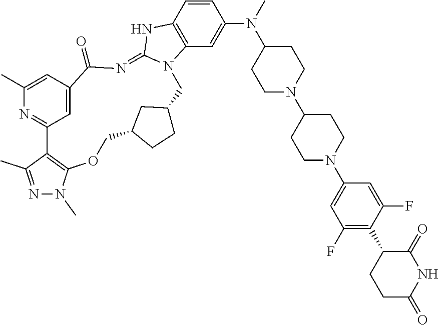 [SMILES: Cc1cc2cc(n1)-c1c(C)nn(C)c1OC[C@H]1CC[C@H](C1)CN1/C(=N/C2=O)Nc2ccc(N(C)C3CCN(C4CCN(c5cc(F)c([C@H]6CCC(=O)NC6=O)c(F)c5)CC4)CC3)cc21]

The title compound was prepared in a manner similar to that in example 1 with intermediates 90 and 7. ¹H NMR (500 MHz, DMSO) δ 12.35 (s, 1H), 10.87 (s, 1H), 8.54 (s, 1H), 7.45 (s, 1H), 7.34 (d, J=8.7 Hz, 1H), 6.84-6.74 (m, 2H), 6.63 (d, J=12.8 Hz, 2H), 4.36-3.97 (m, 5H), 3.81 (d, J=12.3 Hz, 2H), 3.67 (s, 3H), 3.61-3.54 (m, 1H), 2.96 (d, J=9.7 Hz, 2H), 2.81-2.70 (m, 6H), 2.65-2.52 (m, 8H), 2.48 (s, 3H), 2.31-2.23 (m, 2H), 2.13-2.03 (m, 1H), 1.99-1.59 (m, 12H), 1.52-1.42 (m, 2H). [M+H]⁺=875.6.

### Example 144: 3-(4-((3S,4R)-3-fluoro-4-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenoxy)piperidine-2,6-dione

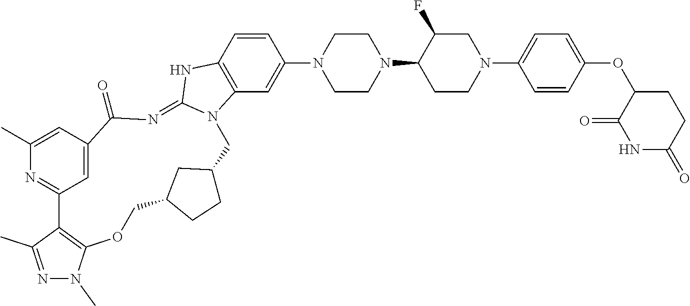 [SMILES: Cc1cc2cc(n1)-c1c(C)nn(C)c1OC[C@H]1CC[C@H](C1)CN1/C(=N/C2=O)Nc2ccc(N3CCN([C@@H]4CCN(c5ccc(OC6CCC(=O)NC6=O)cc5)C[C@@H]4F)CC3)cc21]

The title compound was prepared in a manner similar to that in example 11 with intermediates 39 and 91. ¹H NMR (500 MHz, DMSO) δ 12.43 (s, 1H), 10.88 (s, 1H), 8.54 (s, 1H), 7.45 (s, 1H), 7.37 (d, J=8.7 Hz, 1H), 7.04 (s, 1H), 6.96-6.83 (m, 5H), 5.16-5.06 (m, 1H), 5.02 (dd, J=10.4, 4.9 Hz, 1H), 4.37-4.24 (m, 1H), 4.22-4.15 (m, 1H), 4.14-3.98 (m, 2H), 3.79 (t, J=11.6 Hz, 1H), 3.67 (s, 4H), 3.18 (d, J=4.1 Hz, 4H), 2.87-2.73 (m, 5H), 2.73-2.53 (m, 10H), 2.48 (s, 4H), 2.22-2.14 (m, 1H), 2.12-2.03 (m, 1H), 1.97-1.93 (m, 1H), 1.91-1.74 (m, 4H), 1.66-1.62 (m, 1H); [M+H]⁺=845.7.

### Example 147: 3-(5-(4-((S)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4,6-dimethylpyridin-2-yl)piperidine-2,6-dione

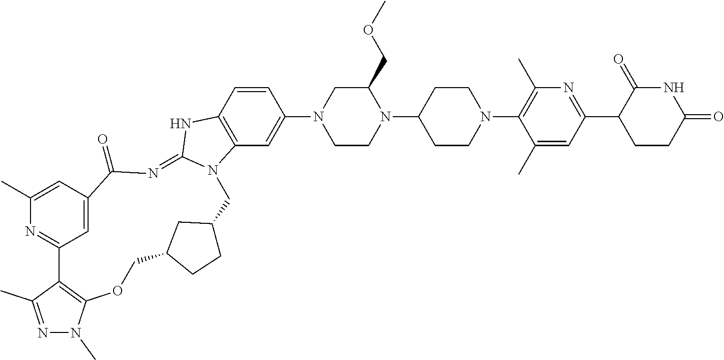 [SMILES: COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(C3CCC(=O)NC3=O)nc2C)CC1]

The title compound was prepared in a manner similar to that in example 1 with intermediates 40 and 13. ¹H NMR (500 MHz, DMSO) δ 12.41 (s, 1H), 10.78 (s, 1H), 8.54 (s, 1H), 7.45 (s, 1H), 7.37 (dd, J=8.7, 4.3 Hz, 1H), 7.03-6.86 (m, 3H), 4.38-3.95 (m, 4H), 3.84 (dd, J=9.1, 5.4 Hz, 1H), 3.67 (s, 3H), 3.61-3.47 (m, 3H), 3.29 (d, J=2.8 Hz, 4H), 3.18-2.78 (m, 9H), 2.74-2.52 (m, 10H), 2.48 (s, 3H), 2.44-2.35 (m, 3H), 2.28 (s, 3H), 2.18 (dt, J=14.4, 7.1 Hz, 1H), 2.11-2.03 (m, 1H), 1.92-1.48 (m, 8H). [M+H]⁺=884.6.

### Example 150: (R)-3-(2,6-difluoro-4-(4-((R)-2-(methoxymethyl)-4-((7S,7³R,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

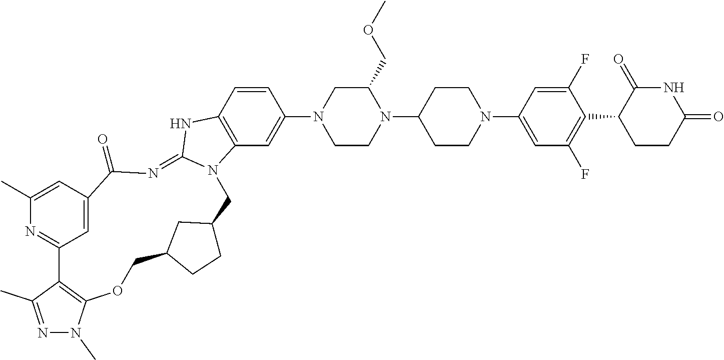 [SMILES: COC[C@H]1CN(c2ccc3c(c2)N2C[C@H]4CC[C@@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 93 and 7. ¹H NMR (500 MHz, DMSO) δ 12.43 (s, 1H), 10.87 (s, 1H), 8.54 (s, 1H), 7.45 (s, 1H), 7.37 (d, J=8.7 Hz, 1H), 6.98 (s, 1H), 6.87 (d, J=8.8 Hz, 1H), 6.65 (s, 1H), 6.63 (s, 1H), 4.38-3.95 (m, 5H), 3.82 (d, J=12.4 Hz, 2H), 3.67 (s, 3H), 3.57 (dd, J=9.7, 4.3 Hz, 1H), 3.50 (dd, J=9.6, 5.8 Hz, 1H), 3.32 (s, 3H), 3.27 (s, 2H), 3.20 (s, 1H), 3.09-2.95 (m, 3H), 2.93-2.86 (m, 1H), 2.85-2.51 (m, 12H), 2.48-2.41 (m, 3H), 2.15-2.03 (m, 1H), 2.00-1.93 (m, 1H), 1.91-1.77 (m, 4H), 1.76-1.69 (m, 1H), 1.67-1.55 (m, 2H), 1.50-1.39 (m, 1H). [M+H]⁺=891.7

### Example 151: (R)-3-(2,6-difluoro-4-(4-((S)-4-((7¹R,7³S,E)-5⁵-methoxy-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)phenyl)piperidine-2,6-dione

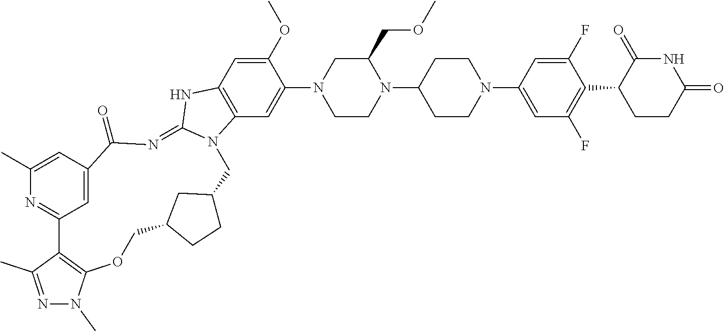 [SMILES: COC[C@@H]1CN(c2cc3c(cc2OC)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

The title compound was prepared in a manner similar to that in example 1 with intermediates 94 and 7. ¹H NMR (500 MHz, DMSO) δ 12.44 (s, 1H), 10.87 (s, 1H), 8.53 (s, 1H), 7.45 (s, 1H), 7.16 (s, 1H), 7.04 (s, 1H), 6.64 (d, J=12.8 Hz, 2H), 4.34-3.98 (m, 5H), 3.84-3.76 (m, 5H), 3.66 (s, 3H), 3.61 (d, J=4.9 Hz, 2H), 3.30 (s, 3H), 3.05-2.66 (m, 12H), 2.62-2.52 (m, 6H), 2.48 (s, 3H), 2.13-2.04 (m, 1H), 1.99-1.71 (m, 7H), 1.63-1.41 (m, 3H). [M+H]⁺=921.6.

### Example 152: (R)-3-(2,6-difluoro-4-(4-(4-((7¹R,7³S,E)-5⁵-methoxy-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

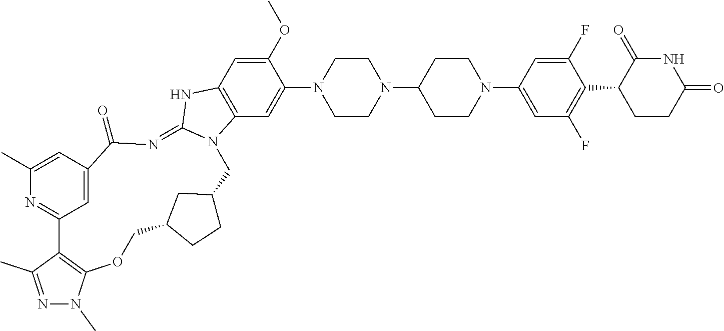 [SMILES: COc1cc2c(cc1N1CCN(C3CCN(c4cc(F)c([C@H]5CCC(=O)NC5=O)c(F)c4)CC3)CC1)N1C[C@@H]3CC[C@H](COc4c(c(C)nn4C)-c4cc(cc(C)n4)C(=O)/N=C/1N2)C3]

The title compound was prepared in a manner similar to that in example 1 with intermediates 95 and 7. ¹H NMR (500 MHz, DMSO) δ 12.44 (s, 1H), 10.87 (s, 1H), 8.53 (s, 1H), 7.45 (s, 1H), 7.16 (s, 1H), 7.03 (s, 1H), 6.64 (d, J=12.8 Hz, 2H), 4.34-3.98 (m, 5H), 3.84-3.76 (m, 5H), 3.66 (s, 3H), 3.06-2.95 (m, 4H), 2.83-2.73 (m, 3H), 2.70-2.51 (m, 12H), 2.48-2.41 (m, 4H), 2.14-2.03 (m, 1H), 2.00-1.70 (m, 7H), 1.49 (d, J=10.6 Hz, 2H). [M+H]⁺=877.6.

### Example 153: (R)-3-(2,6-difluoro-4-(4-(methyl(1-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)azetidin-3-yl)amino)piperidin-1-yl)phenyl)piperidine-2,6-dione

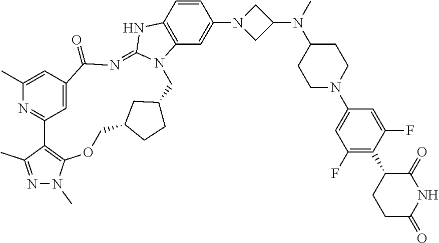 [SMILES: Cc1cc2cc(n1)-c1c(C)nn(C)c1OC[C@H]1CC[C@H](C1)CN1/C(=N/C2=O)Nc2ccc(N3CC(N(C)C4CCN(c5cc(F)c([C@H]6CCC(=O)NC6=O)c(F)c5)CC4)C3)cc21]

The title compound was prepared in a manner similar to that in example 1 with intermediates 96 and 7. ¹H NMR (500 MHz, DMSO) δ 12.38 (s, 1H), 10.87 (s, 1H), 8.53 (s, 1H), 7.45 (s, 1H), 7.33 (s, 1H), 6.64 (d, J=12.7 Hz, 2H), 6.52 (s, 1H), 6.38 (dd, J=8.6, 1.9 Hz, 1H), 4.34-3.97 (m, 7H), 3.85 (d, J=12.1 Hz, 2H), 3.78-3.72 (m, 1H), 3.67 (s, 3H), 3.60 (t, J=6.1 Hz, 2H), 2.81-2.52 (m, 12H), 2.47 (s, 3H), 2.14 (s, 3H), 2.12-2.03 (m, 1H), 1.99-1.64 (m, 7H), 1.55-1.45 (m, 2H). [M+H]⁺=847.6.

### Example 156: (R)-3-(2,6-difluoro-4-(4-((R)-4-((7¹R,7³S,E)-1¹-(2-hydroxyethyl)-1³,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)-2-(methoxymethyl)piperazin-1-yl)piperidin-1-yl)phenyl)piperidine-2,6-dione

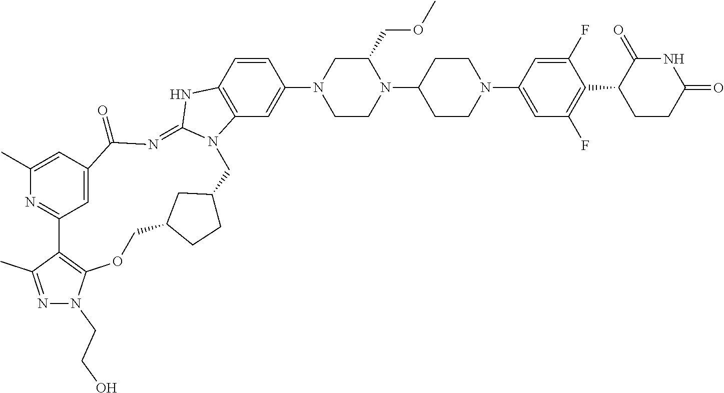 [SMILES: COC[C@H]1CN(c2ccc3c(c2)N2C[C@@H]4CC[C@H](COc5c(c(C)nn5CCO)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 98 and 7. ¹H NMR (500 MHz, DMSO) δ 12.42 (s, 1H), 10.87 (s, 1H), 8.52 (s, 1H), 7.44 (s, 1H), 7.37 (d, J=8.7 Hz, 1H), 6.98 (s, 1H), 6.87 (d, J=8.8 Hz, 1H), 6.64 (d, J=12.7 Hz, 2H), 4.95 (t, J=5.4 Hz, 1H), 4.36-4.21 (m, 2H), 4.11-4.00 (m, 3H), 4.00-3.93 (m, 2H), 3.85-3.81 (m, 4H), 3.59-3.46 (m, 2H), 3.29 (d, J=9.0 Hz, 4H), 3.24-3.14 (m, 1H), 3.08-2.97 (m, 3H), 2.90-2.84 (m, 1H), 2.77-2.74 (m, 4H), 2.68-2.65 (m, 2H), 2.56 (d, J=8.4 Hz, 4H), 2.51 (s, 4H), 2.13-2.03 (m, 1H), 1.99-1.69 (m, 7H), 1.68-1.56 (m, 2H), 1.51-1.39 (m, 1H). [M+H]⁺=921.7.

### Example 157: (R)-3-(2,6-difluoro-4-(4-((S)-4-((7¹R,7³S,E)-1¹-(2-hydroxyethyl)-1³,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)-2-(methoxymethyl)piperazin-1-yl)piperidin-1-yl)phenyl)piperidine-2,6-dione

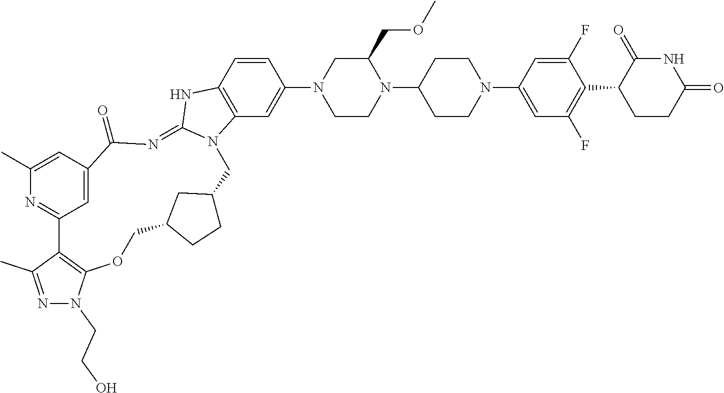 [SMILES: COC[C@@H]1CN(c2ccc3c(c2)N2C[C@@H]4CC[C@H](COc5c(c(C)nn5CCO)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 99 and 7. ¹H NMR (500 MHz, DMSO) δ 12.42 (s, 1H), 10.87 (s, 1H), 8.52 (s, 1H), 7.44 (s, 1H), 7.37 (d, J=8.7 Hz, 1H), 6.98 (s, 1H), 6.87 (d, J=8.8 Hz, 1H), 6.64 (d, J=12.7 Hz, 2H), 4.95 (t, J=5.4 Hz, 1H), 4.36-4.21 (m, 2H), 4.11-4.00 (m, 3H), 4.00-3.93 (m, 2H), 3.85-3.81 (m, 4H), 3.59-3.46 (m, 2H), 3.29 (d, J=9.0 Hz, 4H), 3.24-3.14 (m, 1H), 3.08-2.97 (m, 3H), 2.90-2.84 (m, 1H), 2.77-2.74 (m, 4H), 2.68-2.65 (m, 2H), 2.58 (d, J=8.4 Hz, 4H), 2.51 (s, 4H), 2.13-2.03 (m, 1H), 1.99-1.69 (m, 7H), 1.68-1.57 (m, 2H), 1.51-1.40 (m, 1H). [M+H]⁺=921.7.

### Example 158: 3-(3,5-difluoro-4-(3-((1-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)amino)azetidin-1-yl)phenoxy)piperidine-2,6-dione

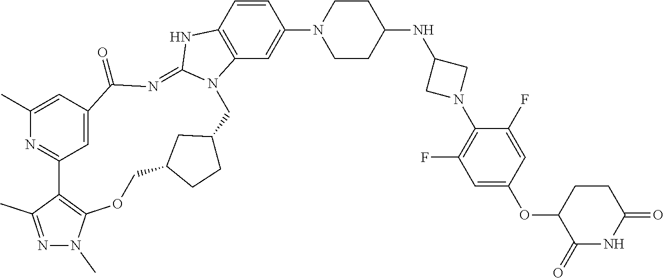 [SMILES: Cc1cc2cc(n1)-c1c(C)nn(C)c1OC[C@H]1CC[C@H](C1)CN1/C(=N/C2=O)Nc2ccc(N3CCC(NC4CN(c5c(F)cc(OC6CCC(=O)NC6=O)cc5F)C4)CC3)cc21]

The title compound was prepared in a manner similar to that in example 1 with intermediates 101 and 100. ¹H NMR (500 MHz, DMSO) δ 12.40 (s, 1H), 10.92 (s, 1H), 8.53 (s, 1H), 7.45 (s, 1H), 7.35 (d, J=8.7 Hz, 1H), 7.01 (s, 1H), 6.90 (d, J=8.8 Hz, 1H), 6.74 (s, 1H), 6.72 (s, 1H), 5.09 (dd, J=11.0, 5.1 Hz, 1H), 4.42-3.94 (m, 6H), 3.76-3.56 (m, 8H), 2.78-2.51 (m, 12H), 2.48-2.38 (m, 3H), 2.21-2.14 (m, 1H), 2.14-2.03 (m, 1H), 1.90-1.60 (m, 6H), 1.45-1.33 (m, 2H). [M+H]⁺=849.7

### Example 159: 3-((5-(4-((R)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4,6-dimethylpyridin-2-yl)amino)piperidine-2,6-dione

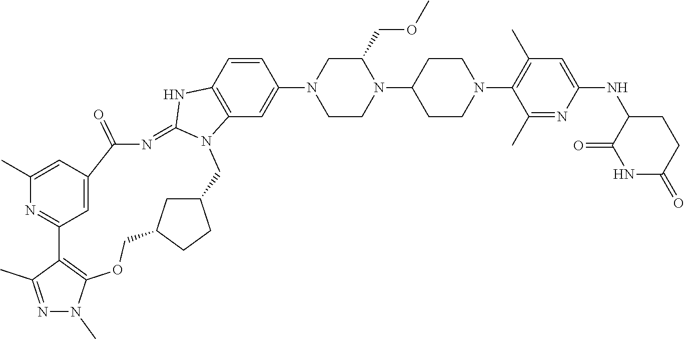 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(NC3CCC(=O)NC3=O)nc2C)CC1]

The title compound was prepared in a manner similar to that in example 1 with intermediates 47 and 102. ¹H NMR (500 MHz, DMSO) δ_{H} 12.43 (s, 1H), 10.72 (s, 1H), 8.54 (s, 1H), 7.45 (s, 1H), 7.37 (d, J=8.7 Hz, 1H), 6.98 (s, 1H), 6.88 (d, J=8.8 Hz, 1H), 6.42-6.35 (m, 1H), 6.25-6.12 (m, 1H), 4.65 (d, J=8.6 Hz, 1H), 4.38-3.95 (m, 4H), 3.67 (s, 3H), 3.58-3.47 (m, 3H), 3.30-2.93 (m, 9H), 2.91-2.52 (m, 14H), 2.48 (s, 3H), 2.31-2.22 (m, 3H), 2.16 (d, J=13.1 Hz, 3H), 2.05-1.95 (m, 2H), 1.90-1.46 (m, 8H). [M+H]⁺=899.6.

### Example 160: (R)-3-(2,6-difluoro-4-(4-((R)-4-((7¹R,7³S,E)-1¹-(2-methoxyethyl)-1³,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)-2-(methoxymethyl)piperazin-1-yl)piperidin-1-yl)phenyl)piperidine-2,6-dione

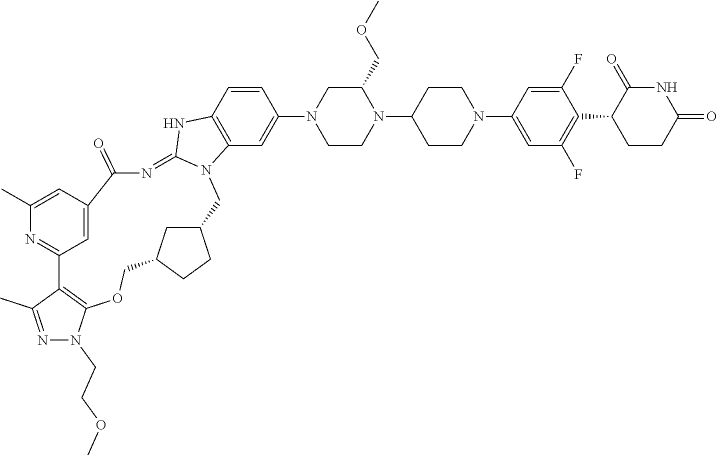 [SMILES: COCCn1nc(C)c2c1OC[C@H]1CC[C@H](C1)CN1/C(=N/C(=O)c3cc(C)nc-2c3)Nc2ccc(N3CCN(C4CCN(c5cc(F)c([C@H]6CCC(=O)NC6=O)c(F)c5)CC4)[C@@H](COC)C3)cc21]

The title compound was prepared in a manner similar to that in example 1 with intermediates 103 and 7. ¹H NMR (500 MHz, DMSO) δ 12.42 (s, 1H), 10.87 (s, 1H), 8.51 (s, 1H), 7.45 (s, 1H), 7.37 (d, J=8.7 Hz, 1H), 6.97 (s, 1H), 6.87 (d, J=8.8 Hz, 1H), 6.65 (s, 1H), 6.63 (s, 1H), 4.40-3.99 (m, 7H), 3.87-3.79 (m, 2H), 3.78-3.70 (m, 2H), 3.60-3.54 (m, 1H), 3.54-3.48 (m, 1H), 3.31-3.17 (m, 9H), 3.09-2.97 (m, 3H), 2.93-2.86 (m, 1H), 2.85-2.53 (m, 12H), 2.49-2.47 (m, 2H), 2.15-2.04 (m, 1H), 2.00-1.94 (m, 1H), 1.92-1.70 (m, 6H), 1.68-1.56 (m, 2H), 1.50-1.39 (m, 1H). [M+H]⁺=935.7

### Example 161: (R)-3-(2,6-difluoro-4-(4-(4-((7¹R,7³S,E)-1¹-(2-methoxyethyl)-1³,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

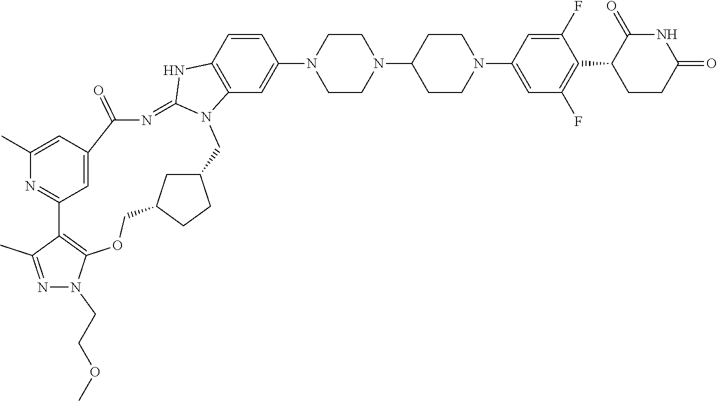 [SMILES: COCCn1nc(C)c2c1OC[C@H]1CC[C@H](C1)CN1/C(=N/C(=O)c3cc(C)nc-2c3)Nc2ccc(N3CCN(C4CCN(c5cc(F)c([C@H]6CCC(=O)NC6=O)c(F)c5)CC4)CC3)cc21]

The title compound was prepared in a manner similar to that in example 1 with intermediates 104 and 7. ¹H NMR (500 MHz, DMSO) δ 12.42 (s, 1H), 10.87 (s, 1H), 8.51 (s, 1H), 7.45 (s, 1H), 7.36 (d, J=8.7 Hz, 1H), 7.01 (s, 1H), 6.93-6.85 (m, 1H), 6.65 (s, 1H), 6.63 (s, 1H), 4.40-4.17 (m, 2H), 4.14-3.95 (m, 5H), 3.85-3.69 (m, 4H), 3.57-3.27 (m, 7H), 3.20-3.09 (m, 4H), 2.83-2.53 (m, 13H), 2.48-2.39 (m, 2H), 2.14-2.03 (m, 1H), 1.99-1.93 (m, 1H), 1.90-1.76 (m, 5H), 1.72-1.59 (m, 1H), 1.53-1.43 (m, 2H). [M+H]⁺=891.7

### Example 162: 3-(5-(4-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-methylpiperazin-1-yl)piperidin-1-yl)-6-methylpyridin-2-yl)piperidine-2,6-dione

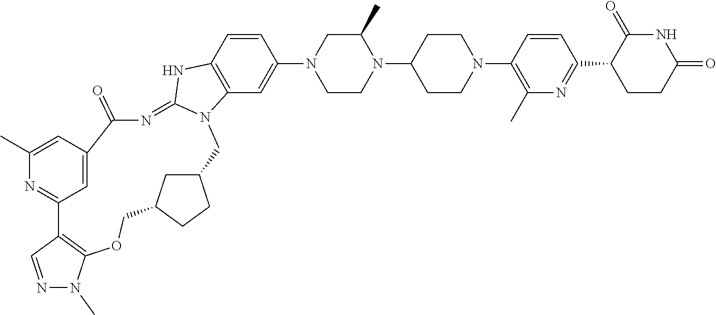 [SMILES: Cc1cc2cc(n1)-c1cnn(C)c1OC[C@H]1CC[C@H](C1)CN1/C(=N/C2=O)Nc2ccc(N3CCN(C4CCN(c5ccc([C@H]6CCC(=O)NC6=O)nc5C)CC4)[C@H](C)C3)cc21]

The title compound was prepared in a manner similar to that in example 1 with intermediates 105 and 15. ¹H NMR (500 MHz, DMSO) δ 12.41 (s, 1H), 10.78 (s, 1H), 8.53 (s, 1H), 7.89 (s, 1H), 7.45 (s, 1H), 7.37 (t, J=8.4 Hz, 2H), 7.11 (d, J=8.2 Hz, 1H), 7.01 (s, 1H), 6.91 (d, J=9.0 Hz, 1H), 4.37-4.24 (m, 1H), 4.19 (dd, J=15.9, 6.3 Hz, 1H), 4.14-4.01 (m, 2H), 3.89 (dd, J=9.3, 5.3 Hz, 1H), 3.74 (s, 3H), 3.51-3.45 (m, 1H), 3.43 (dd, J=7.8, 5.5 Hz, 1H), 3.14 (dd, J=15.4, 6.6 Hz, 2H), 2.96-2.86 (m, 4H), 2.75-2.69 (m, 1H), 2.63-2.54 (m, 11H), 2.45-2.39 (m, 3H), 2.28-2.15 (m, 1H), 2.08 (dd, J=12.9, 6.2 Hz, 1H), 1.98-1.75 (m, 6H), 1.71-1.66 (m, 2H), 1.63-1.57 (m, 1H), 1.13 (d, J=6.2 Hz, 3H). [M+H]⁺=826.7.

### Example 163: 2-((S)-1-(1-(4-((R)-2,6-dioxopiperidin-3-yl)-3,5-difluorophenyl)piperidin-4-yl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-2-yl)acetonitrile

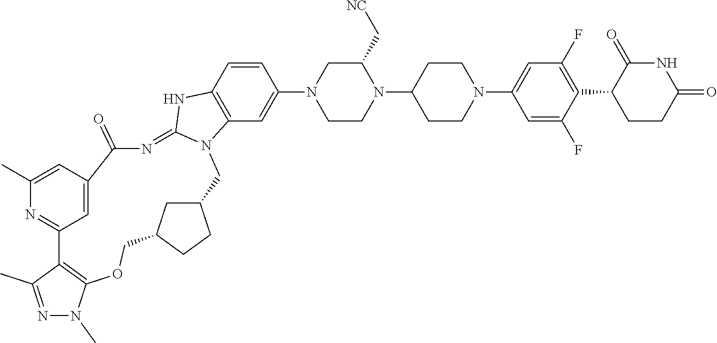 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)[C@@H](CC#N)C3)cc21]

The title compound was prepared in a manner similar to that in example 1 with intermediates 106 and 7. ¹H NMR (500 MHz, DMSO) δ 12.41 (s, 1H), 10.87 (s, 1H), 8.54 (s, 1H), 7.45 (s, 1H), 7.39 (d, J=8.7 Hz, 1H), 7.03 (s, 1H), 6.90 (d, J=9.1 Hz, 1H), 6.66 (s, 1H), 6.64 (s, 1H), 4.35-4.29 (m, 1H), 4.23-4.15 (m, 1H), 4.13-3.98 (m, 3H), 3.87-3.78 (m, 2H), 3.67 (s, 3H), 3.27 (dd, J=7.9, 4.3 Hz, 1H), 3.17-3.10 (m, 3H), 3.09-3.02 (m, 1H), 2.86-2.70 (m, 9H), 2.55 (s, 6H), 2.48-2.43 (m, 4H), 2.10-2.06 (m, 1H), 1.99-1.91 (m, 1H), 1.89-1.75 (m, 5H), 1.68-1.53 (m, 2H), 1.50-1.38 (m, 1H). [M+H]⁺=886.7.

### Example 164: (R)-3-(2,6-difluoro-4-(4-(2-oxo-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

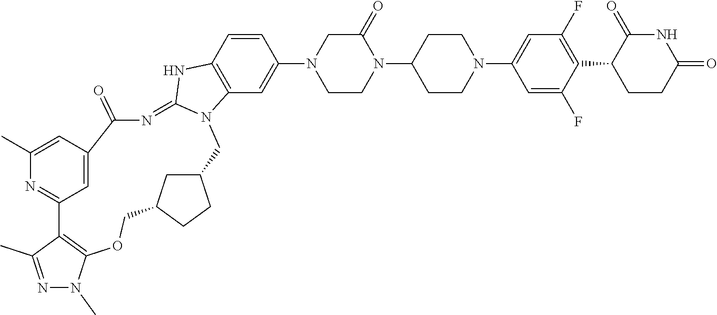 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C(=O)C3)cc21]

The racemate compound was prepared in a manner similar to that in example 11 with intermediates 107 and 39.

The title compound was purified by chiral HPLC and corresponds to the peak @ 15.858 min.

Inj. Volume: 2.0 μL

Column: CHIRALPAK IE 4.6*250 mm 5 μm

Flow Rate (mL/min): 1.0

Gradient:

| Time (min) | MtBE (0.1% DEA + 0.1% FA)(%) | MeOH:DCM = 50:50(%) |
|---|---|---|
| 0 | 20 | 80 |
| 30 | 20 | 80 |

¹H NMR (500 MHz, DMSO) δ 12.44 (s, 1H), 10.88 (s, 1H), 8.54 (s, 1H), 7.45 (s, 1H), 7.39 (d, J=8.7 Hz, 1H), 7.07 (s, 1H), 6.90 (dd, J=8.9, 2.0 Hz, 1H), 6.69 (s, 1H), 6.66 (s, 1H), 4.52-4.57 (m, 1H), 4.34-4.29 (m, 1H), 4.20-4.16 (m, 1H), 4.08-4.04 (m, 2H), 3.91-3.87 (m, 4H), 3.67 (s, 3H), 3.47 (d, J=3.9 Hz, 2H), 3.40 (d, J=5.0 Hz, 2H), 2.88-2.75 (m, 3H), 2.64-2.62 (m, 1H), 2.55 (s, 3H), 2.54-2.51 (m, 4H), 2.48 (s, 3H), 2.11-2.07 (m, 1H), 2.01-1.92 (m, 1H), 1.83-1.76 (m, 6H), 1.64-1.62 (m, 3H). [M+H]⁺=861.7

### Example 165: (R)-3-(2,6-difluoro-4-(4-((R)-4-((7¹R,7³S,E)-5⁵-fluoro-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)phenyl)piperidine-2,6-dione

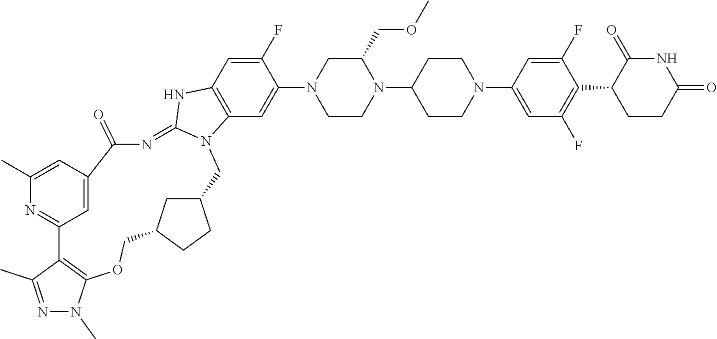 [SMILES: COC[C@H]1CN(c2cc3c(cc2F)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

The title compound was prepared in a manner similar to that in example 1 with intermediates 108 and 7. ¹H NMR (500 MHz, DMSO) δ 12.56 (s, 1H), 10.87 (s, 1H), 8.53 (s, 1H), 7.45 (s, 1H), 7.29 (d, J=11.5 Hz, 1H), 7.18 (d, J=7.3 Hz, 1H), 6.64 (d, J=12.8 Hz, 2H), 4.26-4.22 (m, 1H), 4.18-4.14 (m, 1H), 4.06-4.03 (m, 3H), 3.83 (d, J=11.5 Hz, 2H), 3.70 (d, J=9.7 Hz, 1H), 3.67 (s, 3H), 3.61-3.58 (m, 1H), 3.54 (dd, J=9.6, 4.3 Hz, 1H), 3.42-3.39 (m, 1H), 3.28-3.26 (m, 6H), 2.95-2.87 (m, 2H), 2.79-2.73 (m, 4H), 2.64-2.60 (m, 2H), 2.55 (s, 4H), 2.51-2.47 (m, 5H), 2.13-2.05 (m, 1H), 1.99-1.92 (m, 1H), 1.90-1.79 (m, 4H), 1.73 (d, J=11.3 Hz, 1H), 1.66-1.59 (m, 2H), 1.44-1.42 (m, 1H). [M+H]⁺=909.6

### Example 167: 3-((5-(4-((S)-2-(methoxymethyl)-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-4,6-dimethylpyridin-2-yl)oxy)piperidine-2,6-dione

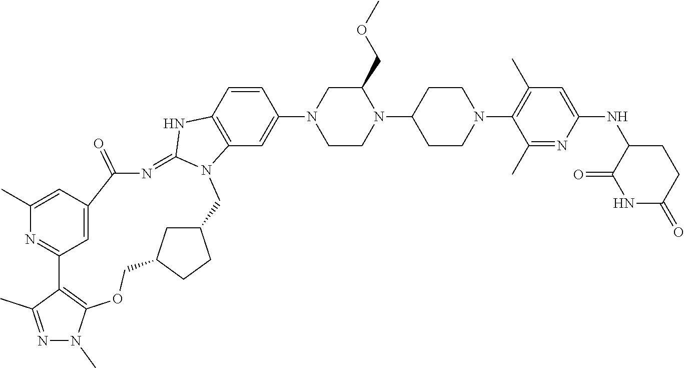 [SMILES: COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(C)cc(NC3CCC(=O)NC3=O)nc2C)CC1]

The title compound was prepared in a manner similar to that in example 1 with intermediates 40 and 109. ¹H NMR (500 MHz, DMSO) δ 12.35 (s, 1H), 10.86 (s, 1H), 8.54 (s, 1H), 7.45 (s, 1H), 7.38 (d, J=8.7 Hz, 1H), 6.99 (s, 1H), 6.88 (d, J=8.7 Hz, 1H), 6.58-6.45 (m, 1H), 5.82 (t, J=8.5 Hz, 1H), 4.38-3.95 (m, 4H), 3.67 (s, 3H), 3.60-3.48 (m, 2H), 3.28-3.01 (m, 7H), 2.99-2.54 (m, 15H), 2.48 (s, 3H), 2.40-2.31 (m, 4H), 2.29-2.23 (m, 3H), 2.17-2.11 (m, 2H), 1.92-1.47 (m, 9H). [M+H]⁺=900.6.

### Example 168: (R)-3-(2,6-difluoro-4-(4-(methyl((4-methyl-1-((7¹R,7³S,E)-1¹,1³′2⁶-trimethyl-3-oxo-5²′5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperidin-4-yl)methyl)amino)piperidin-1-yl)phenyl)piperidine-2,6-dione

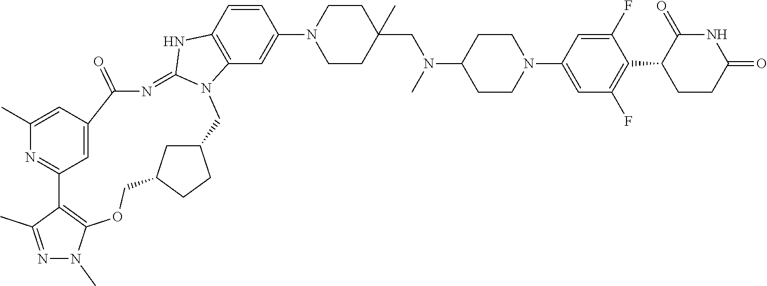 [SMILES: Cc1cc2cc(n1)-c1c(C)nn(C)c1OC[C@H]1CC[C@H](C1)CN1/C(=N/C2=O)Nc2ccc(N3CCC(C)(CN(C)C4CCN(c5cc(F)c([C@H]6CCC(=O)NC6=O)c(F)c5)CC4)CC3)cc21]

The title compound was prepared in a manner similar to that in example 1 with intermediates 110 and 7. ¹H NMR (500 MHz, DMSO) δ 12.35 (s, 1H), 10.86 (s, 1H), 8.53 (s, 1H), 7.45 (s, 1H), 7.35 (d, J=8.7 Hz, 1H), 7.02 (s, 1H), 6.92 (d, J=6.9 Hz, 1H), 6.62 (d, J=12.8 Hz, 2H), 4.36-3.97 (m, 5H), 3.84 (d, J=12.6 Hz, 2H), 3.67 (s, 3H), 3.26 (s, 3H), 2.96 (t, J=10.4 Hz, 2H), 2.82-2.53 (m, 11H), 2.39-2.22 (m, 6H), 2.11-1.93 (m, 3H), 1.90-1.58 (m, 9H), 1.53-1.36 (m, 4H), 0.95 (s, 3H). [M+H]⁺=903.6.

### Example 169: (R)-3-(2,6-difluoro-4-(4-((S)-4-((7¹R,7³S,E)-5⁵-fluoro-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)phenyl)piperidine-2,6-dione

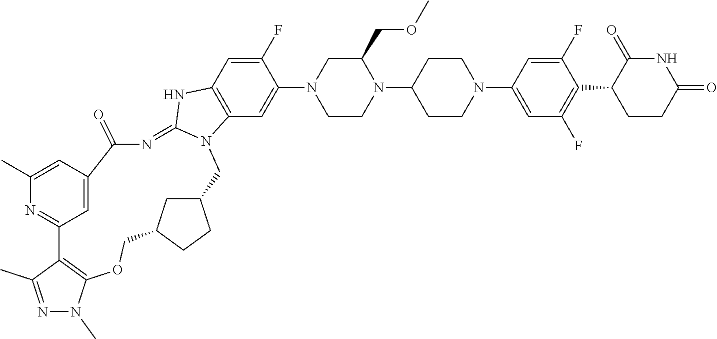 [SMILES: COC[C@@H]1CN(c2cc3c(cc2F)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

The title compound was prepared in a manner similar to that in example 1 with intermediates 111 and 7. ¹H NMR (500 MHz, DMSO) δ 12.56 (s, 1H), 10.87 (s, 1H), 8.53 (s, 1H), 7.45 (s, 1H), 7.29 (d, J=11.5 Hz, 1H), 7.18 (d, J=7.3 Hz, 1H), 6.64 (d, J=12.8 Hz, 2H), 4.26-4.22 (m, 1H), 4.18-4.14 (m, 1H), 4.06-4.03 (m, 3H), 3.83 (d, J=11.5 Hz, 2H), 3.70 (d, J=9.7 Hz, 1H), 3.67 (s, 3H), 3.61-3.58 (m, 1H), 3.54 (dd, J=9.6, 4.3 Hz, 1H), 3.42-3.39 (m, 1H), 3.28-3.26 (m, 6H), 2.95-2.87 (m, 2H), 2.79-2.73 (m, 4H), 2.64-2.60 (m, 2H), 2.55 (s, 4H), 2.51-2.47 (m, 5H), 2.13-2.05 (m, 1H), 1.99-1.92 (m, 1H), 1.90-1.79 (m, 4H), 1.73 (d, J=11.3 Hz, 1H), 1.66-1.59 (m, 2H), 1.44-1.42 (m, 1H). [M+H]⁺=909.6.

### Example 172: (R)-3-(2,6-difluoro-4-(4-((R)-2-(methoxymethyl)-4-((7¹R,7³S,E)-1¹,1³,2⁶° 5⁵-tetramethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

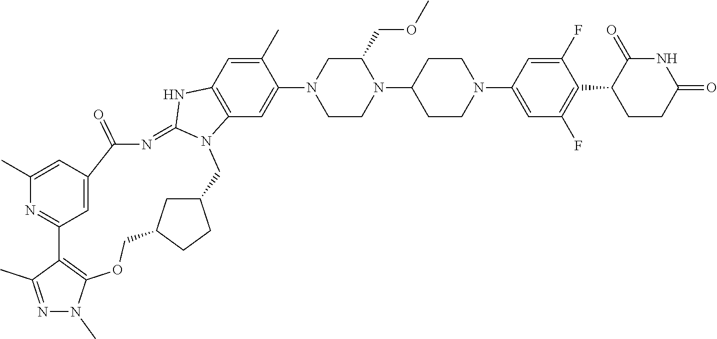 [SMILES: COC[C@H]1CN(c2cc3c(cc2C)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

The title compound was prepare in a manner similar to that in example 1 with intermediates 112 and 7. ¹H NMR (500 MHz, DMSO) δ 12.41 (s, 1H), 10.87 (s, 1H), 8.53 (s, 1H), 7.45 (s, 1H), 7.32 (s, 1H), 7.22 (s, 1H), 6.65 (s, 1H), 6.63 (s, 1H), 4.31 (dd, J=7.3, 5.1 Hz, 1H), 4.18 (t, J=7.3 Hz, 1H), 4.09-3.98 (m, 2H), 3.80 (d, J=12.0 Hz, 2H), 3.66 (s, 3H), 3.60 (d, J=4.4 Hz, 1H), 3.26 (s, 3H), 3.05 (s, 1H), 2.93-2.73 (m, 10H), 2.72-2.65 (m, 1H), 2.64-2.51 (m, 8H), 2.48 (s, 3H), 2.33 (s, 3H), 2.13-2.06 (m, 1H), 1.99-1.71 (m, 7H), 1.69-1.55 (m, 2H), 1.52-1.41 (m, 1H). [M+H]⁺=905.7.

### Example 173: (R)-3-(2,6-difluoro-4-(4-((S)-2-(methoxymethyl)-4-((7¹R,7³S,E)-1¹,1³,2⁶,5⁵-tetramethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

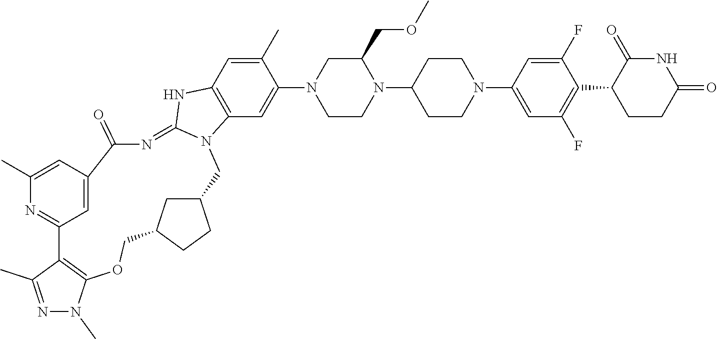 [SMILES: COC[C@@H]1CN(c2cc3c(cc2C)N/C2=N\C(=O)c4cc(C)nc(c4)-c4c(C)nn(C)c4OC[C@H]4CC[C@H](C4)CN23)CCN1C1CCN(c2cc(F)c([C@H]3CCC(=O)NC3=O)c(F)c2)CC1]

The title compound was prepared in a manner similar to that in example 1 with intermediates 113 and 7. ¹H NMR (500 MHz, DMSO) δ 12.41 (s, 1H), 10.87 (s, 1H), 8.53 (s, 1H), 7.45 (s, 1H), 7.32 (s, 1H), 7.22 (s, 1H), 6.65 (s, 1H), 6.63 (s, 1H), 4.31 (dd, J=7.3, 5.1 Hz, 1H), 4.20 (t, J=7.3 Hz, 1H), 4.09-3.98 (m, 2H), 3.82-3.78 (m, 2H), 3.66 (s, 3H), 3.62 (d, J=4.4 Hz, 1H), 3.26 (s, 3H), 3.05 (s, 1H), 2.93-2.73 (m, 10H), 2.72-2.65 (m, 1H), 2.64-2.51 (m, 8H), 2.48 (s, 3H), 2.33 (s, 3H), 2.13-2.06 (m, 1H), 1.99-1.71 (m, 7H), 1.69-1.55 (m, 2H), 1.51-1.40 (m, 1H). [M+H]⁺=905.7.

### Example 175: (R)-3-(4-(4-((R)-4-((7¹R,7³S,E)-1³-ethyl-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

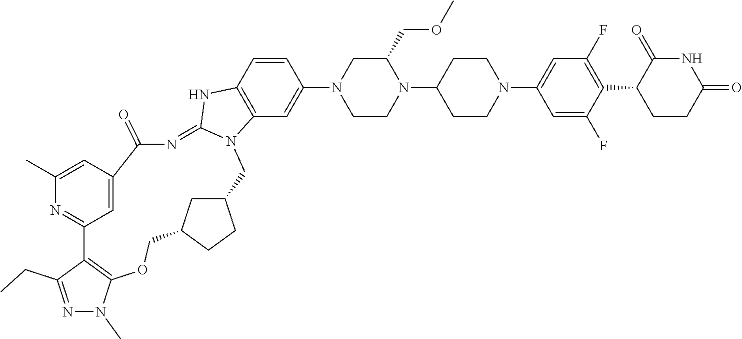 [SMILES: CCc1nn(C)c2c1-c1cc(cc(C)n1)C(=O)/N=C1\Nc3ccc(N4CCN(C5CCN(c6cc(F)c([C@H]7CCC(=O)NC7=O)c(F)c6)CC5)[C@@H](COC)C4)cc3N1C[C@@H]1CC[C@H](CO2)C1]

The title compound was prepared in a manner similar to that in example 1 with intermediates 138 and 7. ¹H NMR (500 MHz, DMSO) δ 12.43 (s, 1H), 10.87 (s, 1H), 8.54 (s, 1H), 7.45 (s, 1H), 7.37 (d, J=8.7 Hz, 1H), 6.98 (s, 1H), 6.87 (d, J=8.6 Hz, 1H), 6.64 (d, J=13.0 Hz, 2H), 4.34-3.98 (m, 5H), 3.83 (d, J=11.6 Hz, 2H), 3.68 (s, 3H), 3.60-3.48 (m, 2H), 3.30-3.18 (m, 6H), 3.06-2.88 (m, 6H), 2.83-2.52 (m, 11H), 2.14-2.04 (m, 1H), 1.96-1.61 (m, 9H), 1.44 (d, J=12.8 Hz, 1H), 1.21-1.16 (m, 3H). [M+H]⁺=905.6.

### Example 176: (R)-3-(4-(4-((R)-4-((7¹R,7³S,E)-2⁶-chloro-1¹,1³-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)piperidin-1-yl)-2,6-difluorophenyl)piperidine-2,6-dione

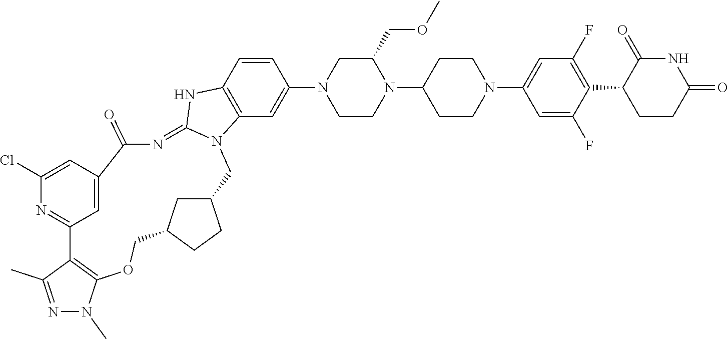 [SMILES: COC[C@H]1CN(c2ccc3c(c2)N2C[C@@H]4CC[C@H](COc5c(c(C)nn5C)-c5cc(cc(Cl)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 139 and 7. ¹H NMR (500 MHz, DMSO) δ 12.52 (s, 1H), 10.87 (s, 1H), 8.69 (s, 1H), 7.55 (s, 1H), 7.38 (d, J=8.7 Hz, 1H), 6.99 (s, 1H), 6.89 (d, J=8.7 Hz, 1H), 6.64 (d, J=12.8 Hz, 2H), 4.31 (s, 1H), 4.24-4.16 (m, 1H), 4.14-4.00 (m, 3H), 3.84-3.78 (m, 2H), 3.68 (s, 3H), 3.57 (dd, J=9.3, 3.8 Hz, 1H), 3.50 (dd, J=6.2, 3.5 Hz, 1H), 3.30 (s, 5H), 3.24-3.16 (m, 1H), 3.03-2.99 (m, 3H), 2.92-2.86 (m, 1H), 2.85-2.71 (m, 4H), 2.70-2.51 (m, 5H), 2.44 (s, 3H), 2.12-2.07 (m, 1H), 1.96-1.93 (m, 1H), 1.91-1.78 (m, 4H), 1.73-1.69 (m, 1H), 1.63-1.59 (m, 2H), 1.49-1.40 (m, 1H). [M+H]⁺=911.7.

### Example 179: (R)-3-(4-(4-(4-((7¹R,7³S,E)-2⁶-chloro-1¹,1³-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

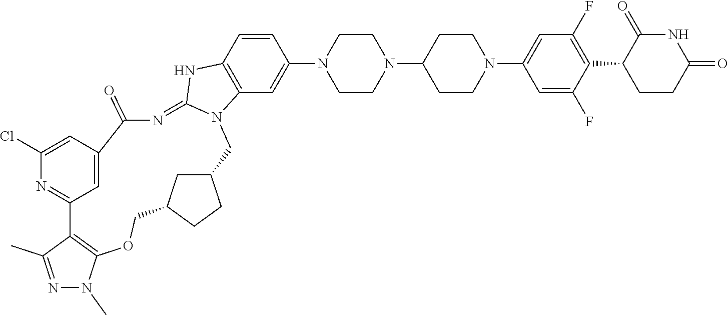 [SMILES: Cc1nn(C)c2c1-c1cc(cc(Cl)n1)C(=O)/N=C1\Nc3ccc(N4CCN(C5CCN(c6cc(F)c([C@H]7CCC(=O)NC7=O)c(F)c6)CC5)CC4)cc3N1C[C@@H]1CC[C@H](CO2)C1]

The title compound was prepared in a manner similar to that in example 1 with intermediates 114 and 7. ¹H NMR (500 MHz, DMSO) δ 12.50 (s, 1H), 10.87 (s, 1H), 8.69 (s, 1H), 7.55 (s, 1H), 7.38 (d, J=8.7 Hz, 1H), 7.04 (s, 1H), 6.93 (d, J=7.2 Hz, 1H), 6.64 (d, J=12.8 Hz, 2H), 4.33-4.28 (m, 1H), 4.24-4.15 (m, 1H), 4.14-4.00 (m, 3H), 3.82 (d, J=11.7 Hz, 2H), 3.68 (s, 3H), 3.23-3.16 (m, 4H), 2.82-2.77 (m, 4H), 2.71-2.59 (m, 7H), 2.44 (s, 4H), 2.16-2.02 (m, 1H), 1.98 (d, J=5.2 Hz, 1H), 1.91-1.72 (m, 6H), 1.66-1.62 (m, 1H), 1.55-1.44 (m, 2H). [M+H]⁺=867.7.

### Example 182: (R)-3-(2,6-difluoro-4-(4-((4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)piperidin-1-yl)phenyl)piperidine-2,6-dione

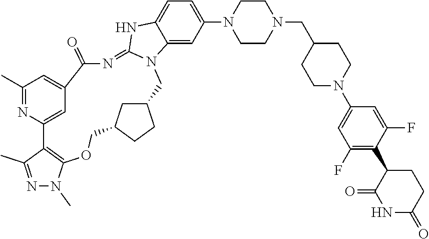 [SMILES: Cc1cc2cc(n1)-c1c(C)nn(C)c1OC[C@H]1CC[C@H](C1)CN1/C(=N/C2=O)Nc2ccc(N3CCN(CC4CCN(c5cc(F)c([C@H]6CCC(=O)NC6=O)c(F)c5)CC4)CC3)cc21]

The title compound was prepared in a manner similar to that in example 1 with intermediates 34 and 80. ¹H NMR (500 MHz, DMSO) δ 12.42 (s, 1H), 10.87 (s, 1H), 8.54 (s, 1H), 7.45 (s, 1H), 7.37 (d, J=8.7 Hz, 1H), 7.03 (s, 1H), 6.91 (d, J=8.9 Hz, 1H), 6.62 (d, J=12.8 Hz, 2H), 4.24 (m, 2H), 4.04 (dd, J=12.6, 5.1 Hz, 2H), 3.76 (d, J=12.7 Hz, 2H), 3.67 (s, 3H), 3.17 (s, 4H), 2.75 (t, J=11.8 Hz, 3H), 2.64 (s, 1H), 2.56-2.54 (m, 5H), 2.53-2.51 (m, 6H), 2.48 (s, 4H), 2.23 (d, J=6.4 Hz, 2H), 2.13-2.05 (m, 1H), 1.99-1.94 (m, 1H), 1.81-1.78 (m, 6H), 1.63 (s, 1H), 1.16 (d, J=11.6 Hz, 2H). [M+H]⁺=861.6.

### Example 183: (R)-3-(2,6-difluoro-4-(4-(2-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)ethyl)piperidin-1-yl)phenyl)piperidine-2,6-dione

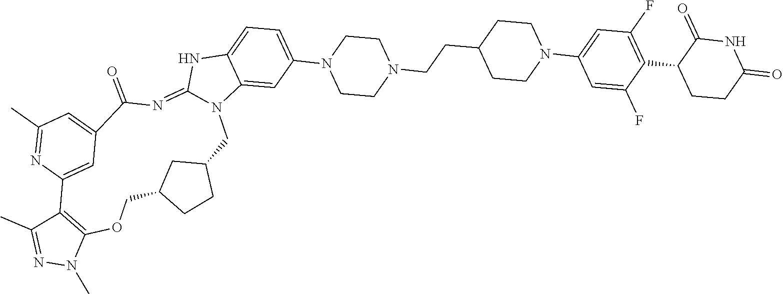 [SMILES: Cc1cc2cc(n1)-c1c(C)nn(C)c1OC[C@H]1CC[C@H](C1)CN1/C(=N/C2=O)Nc2ccc(N3CCN(CCC4CCN(c5cc(F)c([C@H]6CCC(=O)NC6=O)c(F)c5)CC4)CC3)cc21]

The title compound was prepared in a manner similar to that in example 1 with intermediates 34 and 115. ¹H NMR (500 MHz, DMSO) δ 12.42 (s, 1H), 10.86 (s, 1H), 8.54 (s, 1H), 7.45 (s, 1H), 7.36 (d, J=8.7 Hz, 1H), 7.02 (s, 1H), 6.90 (dd, J=8.8, 1.8 Hz, 1H), 6.61 (d, J=12.8 Hz, 2H), 4.30-4.24 (m, 2H), 4.04 (dd, J=12.5, 5.0 Hz, 2H), 3.75 (d, J=12.8 Hz, 2H), 3.67 (s, 3H), 3.16 (s, 4H), 2.82-2.76 (m, 1H), 2.74-2.69 (m, 3H), 2.63 (d, J=1.8 Hz, 1H), 2.57-2.52 (m, 9H), 2.48 (s, 4H), 2.39 (t, J=7.3 Hz, 2H), 2.12-2.03 (m, 1H), 1.98-1.93 (m, 1H), 1.81 (s, 3H), 1.74 (d, J=11.1 Hz, 3H), 1.62 (s, 1H), 1.52 (s, 1H), 1.44 (dd, J=14.0, 6.7 Hz, 2H), 1.23-1.17 (m, 2H). [M+H]⁺=875.7

### Example 184: (R)-3-(2,6-difluoro-4-((S)-3-((4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)pyrrolidin-1-yl)phenyl)piperidine-2,6-dione

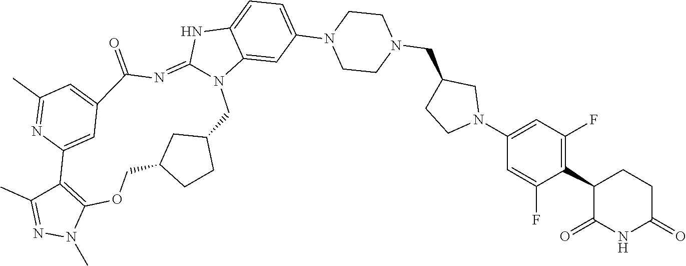 [SMILES: Cc1cc2cc(n1)-c1c(C)nn(C)c1OC[C@H]1CC[C@H](C1)CN1/C(=N/C2=O)Nc2ccc(N3CCN(C[C@@H]4CCN(c5cc(F)c([C@H]6CCC(=O)NC6=O)c(F)c5)C4)CC3)cc21]

The title compound was prepared in a manner similar to that in example 1 with intermediates 34 and 116. ¹H NMR (500 MHz, DMSO) δ 12.42 (s, 1H), 10.84 (s, 1H), 8.54 (s, 1H), 7.45 (s, 1H), 7.37 (d, J=8.7 Hz, 1H), 7.04 (s, 1H), 6.93-6.89 (m, 1H), 6.20 (d, J=12.2 Hz, 2H), 4.30-4.19 (m, 2H), 4.01 (dd, J=12.4, 4.9 Hz, 2H), 3.67 (s, 3H), 3.38 (s, 1H), 3.29 (s, 1H), 3.22 (d, J=8.3 Hz, 1H), 3.19 (s, 4H), 3.02-2.97 (m, 1H), 2.80-2.74 (m, 1H), 2.63 (dd, J=3.7, 1.8 Hz, 2H), 2.61 (s, 3H), 2.58 (s, 2H), 2.55 (s, 3H), 2.52 (d, J=1.9 Hz, 1H), 2.48 (s, 4H), 2.40 (d, J=7.4 Hz, 2H), 2.37-2.35 (m, 1H), 2.09 (t, J=9.6 Hz, 2H), 1.96-1.92 (m, 1H), 1.81 (s, 3H), 1.74 (dd, J=11.8, 7.8 Hz, 2H), 1.63 (s, 1H). [M+H]⁺=847.7

### Example 186: (R)-3-(2,6-difluoro-4-((S)-3-(4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)pyrrolidin-1-yl)phenyl)piperidine-2,6-dione

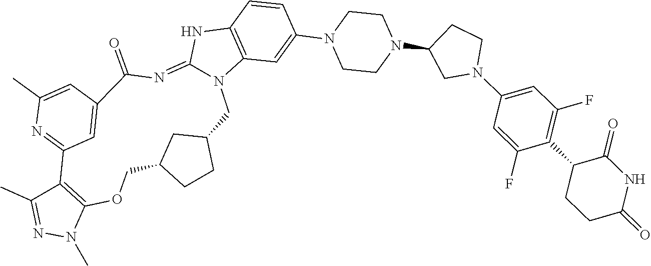 [SMILES: Cc1cc2cc(n1)-c1c(C)nn(C)c1OC[C@H]1CC[C@H](C1)CN1/C(=N/C2=O)Nc2ccc(N3CCN([C@H]4CCN(c5cc(F)c([C@H]6CCC(=O)NC6=O)c(F)c5)C4)CC3)cc21]

The racemate compound was prepared in a manner similar to that in example 11 with intermediates 39 and 117.

The title compound was purified by chiral HPLC, and corresponds to peak A @ 1.773 min

-T

| Column | CHIRALPAK IF |
|---|---|
| Column Size | 4.6*50 mm, 3 μm |
| Mobile Phase A | MtBE(0.1% FA):(MeOH:DCM = 1:1) = 10:90 |
| Flow rate | 1.0 mL/min |
| Temperature | 25° C. |

¹H NMR (500 MHz, DMSO) δ 10.85 (s, 1H), 8.54 (s, 1H), 7.49 (d, J=8.0 Hz, 1H), 7.45 (s, 1H), 7.37 (d, J=8.8 Hz, 1H), 7.04 (s, 1H), 6.92 (d, J=8.4 Hz, 1H), 6.27 (s, 1H), 6.24 (s, 1H), 4.39-3.94 (m, 5H), 3.71-3.65 (m, 3H), 3.63-3.57 (m, 1H), 3.55-3.48 (m, 2H), 3.41-3.35 (m, 3H), 3.25-3.17 (m, 5H), 3.13-3.08 (m, 1H), 3.05-2.96 (m, 1H), 2.85-2.74 (m, 1H), 2.68-2.63 (m, 4H), 2.57-2.54 (m, 3H), 2.48-2.44 (m, 3H), 2.29-2.19 (m, 1H), 2.16-2.03 (m, 1H), 1.98-1.78 (m, 5H), 1.68-1.56 (m, 1H), 1.49-1.42 (m, 1H). [M+H]⁺=833.7.

### Example 188: (R)-3-(4-(4-((R)-2-ethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

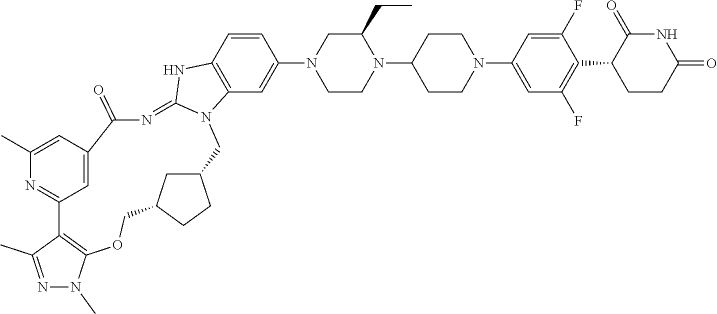 [SMILES: CC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 118 and 7. ¹H NMR (500 MHz, DMSO) δ 12.38 (s, 1H), 10.87 (s, 1H), 8.54 (s, 1H), 7.45 (s, 1H), 7.37 (d, J=8.7 Hz, 1H), 6.99 (d, J=1.4 Hz, 1H), 6.90 (dd, J=8.8, 1.8 Hz, 1H), 6.65 (s, 1H), 6.62 (s, 1H), 4.38-3.97 (m, 5H), 3.82 (d, J=12.2 Hz, 2H), 3.67 (s, 3H), 3.31-3.22 (m, 3H), 2.99 (t, J=8.4 Hz, 1H), 2.94-2.74 (m, 7H), 2.65-2.53 (m, 8H), 2.48-2.34 (m, 3H), 2.14-2.05 (m, 1H), 2.00-1.93 (m, 1H), 1.87-1.59 (m, 9H), 1.48-1.39 (m, 1H), 0.94 (t, J=7.4 Hz, 3H). [M+H]⁺=875.7

### Example 189: (R)-3-(4-(4-((S)-2-ethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

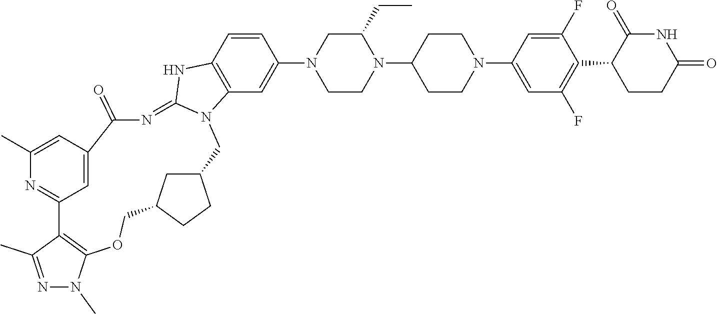 [SMILES: CC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1]

The title compound was prepared in a manner similar to that in example 1 with intermediates 119 and 7. ¹H NMR (500 MHz, DMSO) δ 12.37 (s, 1H), 10.86 (s, 1H), 8.53 (s, 1H), 7.45 (s, 1H), 7.37 (d, J=8.7 Hz, 1H), 6.98 (s, 1H), 6.89 (d, J=8.8 Hz, 1H), 6.64 (s, 1H), 6.62 (s, 1H), 4.41-3.97 (m, 5H), 3.82 (d, J=12.4 Hz, 2H), 3.66 (s, 3H), 3.29-3.17 (m, 3H), 3.03-2.74 (m, 8H), 2.67-2.51 (m, 8H), 2.48-2.41 (m, 3H), 2.14-2.03 (m, 1H), 1.99-1.92 (m, 1H), 1.92-1.60 (m, 9H), 1.49-1.39 (m, 1H), 0.94 (t, J=7.4 Hz, 3H). [M+H]+=875.7

### Example 190: (R)-3-(2,6-difluoro-4-(4-((R)-3-methyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

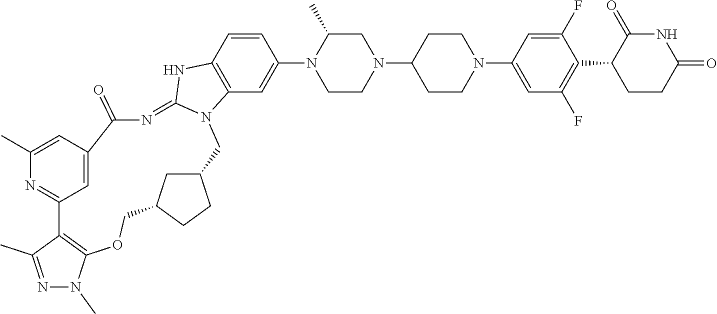 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C[C@H]3C)cc21]

The title compound was prepared in a manner similar to that in example 1 with intermediates 120 and 7. ¹H NMR (500 MHz, DMSO) δ 12.41 (s, 1H), 10.87 (s, 1H), 8.54 (s, 1H), 7.45 (s, 1H), 7.37 (d, J=8.7 Hz, 1H), 7.01 (s, 1H), 6.89 (d, J=7.6 Hz, 1H), 6.65 (s, 1H), 6.63 (s, 1H), 4.37-3.97 (m, 5H), 3.88 (d, J=5.5 Hz, 1H), 3.79 (d, J=12.2 Hz, 2H), 3.66 (s, 3H), 3.21 (d, J=11.6 Hz, 1H), 3.00 (t, J=8.9 Hz, 1H), 2.89-2.73 (m, 4H), 2.68-2.52 (m, 8H), 2.48-2.38 (m, 6H), 2.15-2.03 (m, 1H), 2.00-1.93 (m, 1H), 1.91-1.58 (m, 7H), 1.55-1.41 (m, 2H), 0.96 (d, J=6.3 Hz, 3H). [M+H]⁺=861.7

### Example 191: (R)-3-(4-(4-((2S,5R)-2,5-dimethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

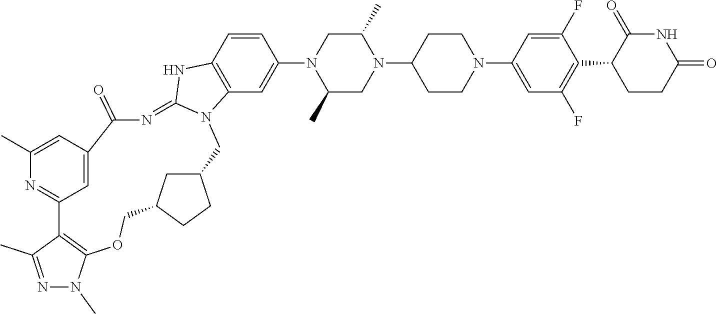 [SMILES: Cc1cc2cc(n1)-c1c(C)nn(C)c1OC[C@H]1CC[C@H](C1)CN1/C(=N/C2=O)Nc2ccc(N3C[C@H](C)N(C4CCN(c5cc(F)c([C@H]6CCC(=O)NC6=O)c(F)c5)CC4)C[C@H]3C)cc21]

The title compound was prepared in a manner similar to that in example 1 with intermediates 121 and 7. ¹H NMR (500 MHz, DMSO) δ 12.48 (s, 1H), 10.86 (s, 1H), 8.54 (s, 1H), 7.45 (s, 1H), 7.40 (d, J=8.5 Hz, 1H), 7.15 (s, 1H), 6.97 (d, J=8.5 Hz, 1H), 6.65 (s, 1H), 6.62 (s, 1H), 4.40-3.96 (m, 5H), 3.81 (d, J=11.8 Hz, 2H), 3.66 (s, 3H), 3.50-3.41 (m, 1H), 3.28 (s, 1H), 3.12-2.99 (m, 2H), 2.92-2.74 (m, 6H), 2.65-2.52 (m, 6H), 2.48 (s, 3H), 2.38-2.30 (m, 1H), 2.14-2.03 (m, 1H), 2.02-1.93 (m, 1H), 1.91-1.58 (m, 8H), 1.51-1.39 (m, 1H), 1.04 (d, J=5.5 Hz, 3H), 0.88 (d, J=6.1 Hz, 3H). [M+H]⁺=875.7

### Example 192: (R)-3-(2,6-difluoro-4-(4-((R)-2-methyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

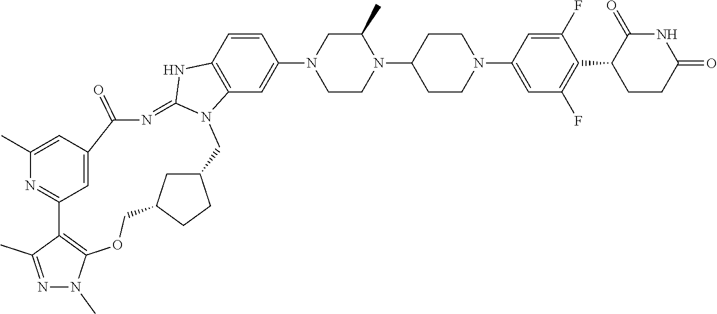 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)[C@H](C)C3)cc21]

The title compound was prepared in a manner similar to that in example 1 with intermediates 84 and 7. ¹H NMR (500 MHz, DMSO) δ 12.42 (s, 1H), 10.87 (s, 1H), 8.54 (s, 1H), 7.45 (s, 1H), 7.36 (d, J=8.7 Hz, 1H), 6.99 (s, 1H), 6.89 (d, J=8.8 Hz, 1H), 6.64 (d, J=12.7 Hz, 2H), 4.29-4.19 (m, 2H), 4.05 (dd, J=12.6, 4.8 Hz, 2H), 3.84 (d, J=12.3 Hz, 2H), 3.67 (s, 3H), 3.40 (t, J=11.2 Hz, 2H), 2.94-2.89 (m, 3H), 2.86-2.73 (m, 5H), 2.63 (t, J=9.7 Hz, 2H), 2.55 (s, 4H), 2.53-2.49 (m, 2H), 2.47 (s, 4H), 2.12-2.05 (m, 1H), 1.98-1.94 (m, 1H), 1.86-1.76 (m, 5H), 1.71-1.60 (m, 3H), 1.42 (d, J=9.7 Hz, 1H), 1.13 (d, J=6.1 Hz, 3H); [M+H]+=861.7.

### Example 193: (3R)-3-(2,6-difluoro-4-(4-(5-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2,5-diazabicyclo[2.2.2]octan-2-yl)piperidin-1-yl)phenyl)piperidine-2,6-dione

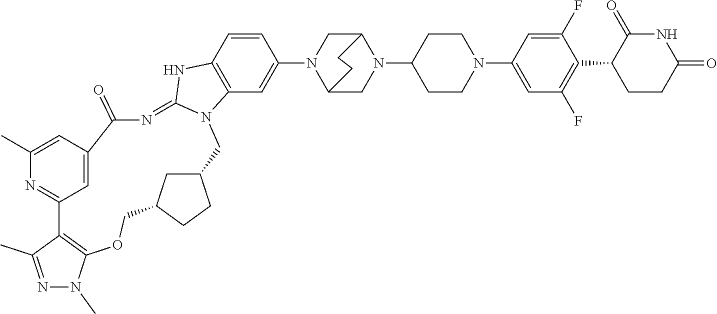 [SMILES: Cc1cc2cc(n1)-c1c(C)nn(C)c1OC[C@H]1CC[C@H](C1)CN1/C(=N/C2=O)Nc2ccc(N3CC4CCC3CN4C3CCN(c4cc(F)c([C@H]5CCC(=O)NC5=O)c(F)c4)CC3)cc21]

The title compound was prepared in a manner similar to that in example 1 with intermediates 122 and 7. ¹H NMR (500 MHz, DMSO) δ 12.32 (s, 1H), 10.86 (s, 1H), 8.53 (s, 1H), 7.45 (s, 1H), 7.32 (d, J=8.7 Hz, 1H), 6.65 (d, J=10.6 Hz, 2H), 6.61 (s, 2H), 4.26-4.02 (m, 2H), 4.07-3.99 (m, 4H), 3.68 (s, 1H), 3.67 (s, 4H), 3.60 (s, 1H), 3.23 (s, 2H), 2.96 (s, 2H), 2.87-2.82 (m, 2H), 2.79-2.74 (m, 1H), 2.66-2.61 (m, 1H), 2.58-2.56 (m, 2H), 2.55 (s, 4H), 2.53-2.52 (m, 1H), 2.47 (s, 4H), 2.10-2.04 (m, 1H), 1.97-1.94 (m, 3H), 1.89-1.86 (m, 3H), 1.81-1.78 (m, 3H), 1.59 (s, 2H), 1.38 (d, J=10.5 Hz, 2H). [M+H]⁺=873.7

### Example 195: (R)-3-(2,6-difluoro-4-(4-((S)-3-methyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)phenyl)piperidine-2,6-dione

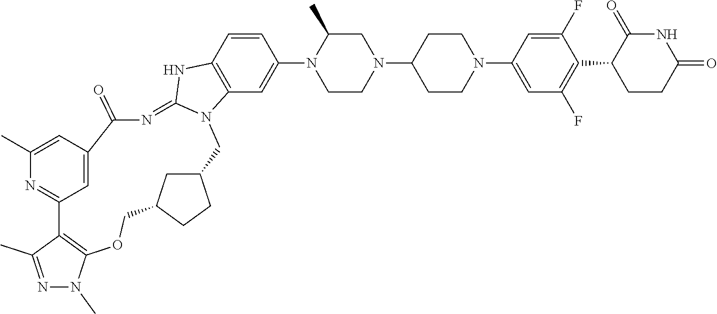 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C[C@@H]3C)cc21]

The title compound was prepared in a manner similar to that in example 1 with intermediates 123 and 7. ¹H NMR (500 MHz, DMSO) δ 12.43 (s, 1H), 10.87 (s, 1H), 8.46 (s, 1H), 7.46 (s, 1H), 7.36 (d, J=8.7 Hz, 1H), 7.03 (s, 1H), 6.90 (d, J=8.7 Hz, 1H), 6.66 (s, 1H), 6.63 (s, 1H), 4.26-4.20 (m, 2H), 4.07-4.03 (m, 2H), 3.82 (d, J=12.4 Hz, 2H), 3.60 (s, 3H), 3.29 (s, 2H), 3.16 (s, 3H), 2.89 (s, 1H), 2.79-2.74 (m, 3H), 2.73-2.72 (m, 6H), 2.70-2.67 (m, 4H), 2.64-2.63 (m, 2H), 2.54 (s, 3H), 2.45 (s, 1H), 2.37-2.35 (m, 1H), 2.11-2.07 (m, 1H), 1.99-1.93 (m, 1H), 1.88 (d, J=11.4 Hz, 3H), 1.75 (s, 2H), 1.49 (d, J=10.1 Hz, 3H). [M+H]⁺=861.5.

### Example 196: (R)-3-(4-(4-((2R,5R)-2,5-dimethyl-4-((7¹R,7³S,E)-1¹,1³2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

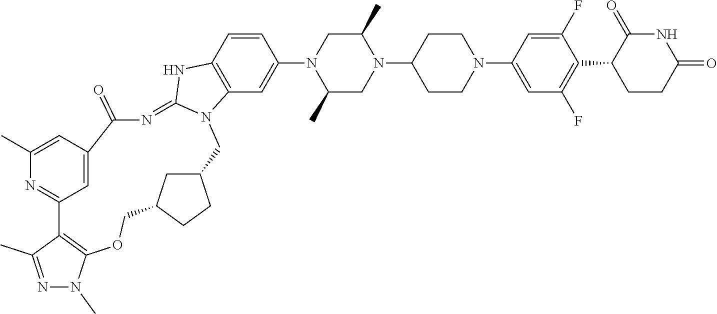 [SMILES: Cc1cc2cc(n1)-c1c(C)nn(C)c1OC[C@H]1CC[C@H](C1)CN1/C(=N/C2=O)Nc2ccc(N3C[C@@H](C)N(C4CCN(c5cc(F)c([C@H]6CCC(=O)NC6=O)c(F)c5)CC4)C[C@H]3C)cc21]

The title compound was prepared in a manner similar to that in example 1 with intermediates 124 and 7. ¹H NMR (500 MHz, DMSO) δ 12.48 (s, 1H), 10.86 (s, 1H), 8.54 (s, 1H), 7.45 (s, 1H), 7.40 (d, J=8.5 Hz, 1H), 7.15 (s, 1H), 6.97 (d, J=8.5 Hz, 1H), 6.65 (s, 1H), 6.62 (s, 1H), 4.40-3.96 (m, 5H), 3.81 (d, J=11.8 Hz, 2H), 3.66 (s, 3H), 3.50-3.41 (m, 1H), 3.28 (s, 1H), 3.12-2.99 (m, 2H), 2.92-2.74 (m, 6H), 2.65-2.52 (m, 6H), 2.48 (s, 3H), 2.38-2.30 (m, 1H), 2.14-2.03 (m, 1H), 2.02-1.93 (m, 1H), 1.91-1.58 (m, 8H), 1.51-1.39 (m, 1H), 1.04 (d, J=5.5 Hz, 3H), 0.88 (d, J=6.1 Hz, 3H). [M+H]⁺=875.6.

### Example 197: (R)-3-(4-(4-((2S,5S)-2,5-dimethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

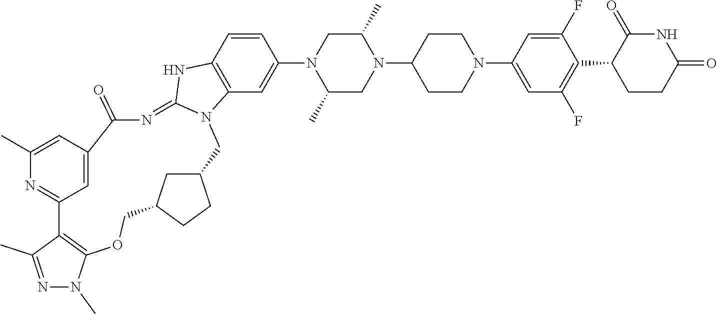 [SMILES: Cc1cc2cc(n1)-c1c(C)nn(C)c1OC[C@H]1CC[C@H](C1)CN1/C(=N/C2=O)Nc2ccc(N3C[C@H](C)N(C4CCN(c5cc(F)c([C@H]6CCC(=O)NC6=O)c(F)c5)CC4)C[C@@H]3C)cc21]

The title compound was prepared in a manner similar to that in example 1 with intermediates 125 and 7. ¹H NMR (500 MHz, DMSO) δ 12.47 (s, 1H), 10.86 (s, 1H), 8.54 (s, 1H), 7.45 (s, 1H), 7.39 (d, J=8.6 Hz, 1H), 7.15 (s, 1H), 6.96 (d, J=8.3 Hz, 1H), 6.65 (s, 1H), 6.62 (s, 1H), 4.39-3.98 (m, 5H), 3.81 (d, J=10.7 Hz, 2H), 3.67 (s, 3H), 3.54-3.45 (m, 1H), 3.28 (s, 1H), 3.12-3.00 (m, 2H), 2.87-2.74 (m, 6H), 2.59-2.52 (m, 7H), 2.48 (s, 3H), 2.15-2.04 (m, 1H), 2.00-1.94 (m, 1H), 1.90-1.62 (m, 8H), 1.51-1.41 (m, 1H), 1.04 (d, J=5.9 Hz, 3H), 0.88 (d, J=6.0 Hz, 3H). [M+H]⁺=875.6.

### Example 198: (R)-3-(4-(4-((3S,5S)-3,5-dimethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

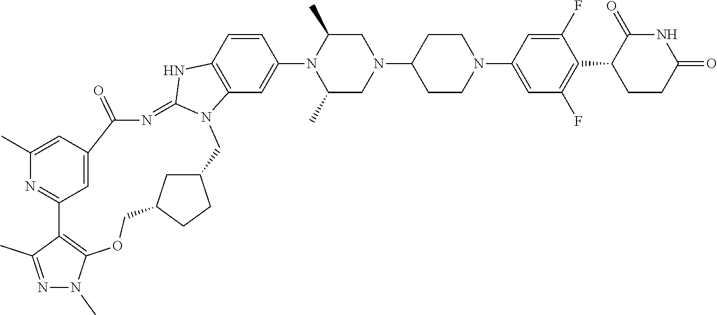 [SMILES: Cc1cc2cc(n1)-c1c(C)nn(C)c1OC[C@H]1CC[C@H](C1)CN1/C(=N/C2=O)Nc2ccc(N3[C@@H](C)CN(C4CCN(c5cc(F)c([C@H]6CCC(=O)NC6=O)c(F)c5)CC4)C[C@@H]3C)cc21]

The title compound was prepared in a manner similar to that in example 1 with intermediates 126 and 7. ¹H NMR (500 MHz, DMSO) δ 12.43 (s, 1H), 10.87 (s, 1H), 8.54 (s, 1H), 7.45 (s, 1H), 7.40 (d, J=8.5 Hz, 1H), 7.15 (s, 1H), 6.93 (d, J=7.6 Hz, 1H), 6.64 (d, J=12.8 Hz, 2H), 4.35-4.30 (m, 1H), 4.20-4.17 (m, 1H), 4.07-4.03 (m, 3H), 3.79-3.77 (m, 2H), 3.67 (s, 3H), 3.58-3.55 (m, 3H), 2.82-2.78 (m, 6H), 2.64-2.62 (m, 4H), 2.43-2.39 (m, 5H), 2.37-2.36 (m, 2H), 2.10-2.07 (m, 1H), 1.98-1.95 (m, 1H), 1.85-1.81 (m, 5H), 1.69-1.64 (m, 1H), 1.53-1.49 (m, 2H), 1.24 (s, 1H), 0.89 (d, J=6.1 Hz, 6H). [M+H]⁺=875.6.

### Example 199: (R)-3-(2,6-difluoro-4-(3-((4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)azetidin-1-yl)phenyl)piperidine-2,6-dione

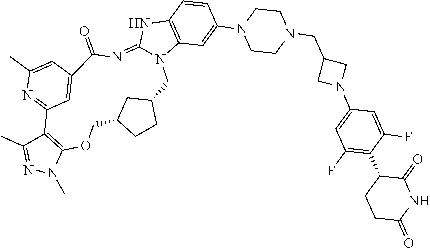 [SMILES: Cc1cc2cc(n1)-c1c(C)nn(C)c1OC[C@H]1CC[C@H](C1)CN1/C(=N/C2=O)Nc2ccc(N3CCN(CC4CN(c5cc(F)c([C@H]6CCC(=O)NC6=O)c(F)c5)C4)CC3)cc21]

The title compound was prepared in a manner similar to that in example 1 with intermediates 34 and 44. ¹H NMR (500 MHz, DMSO) δ 12.42 (s, 1H), 10.85 (s, 1H), 8.54 (s, 1H), 7.45 (s, 1H), 7.37 (d, J=8.7 Hz, 1H), 7.03 (s, 1H), 6.91 (dd, J=8.8, 1.8 Hz, 1H), 6.12 (d, J=11.1 Hz, 2H), 4.32-4.24 (m, 2H), 4.03 (dd, J=12.5, 4.9 Hz, 2H), 3.96 (t, J=7.6 Hz, 2H), 3.67 (s, 3H), 3.51 (t, J=5.9 Hz, 2H), 3.17 (s, 4H), 3.00-2.95 (m, 1H), 2.82-2.76 (m, 1H), 2.64 (d, J=7.2 Hz, 2H), 2.58-2.57 (m, 3H), 2.56-2.55 (m, 6H), 2.53-2.52 (m, 2H), 2.48 (s, 4H), 2.09-2.03 (m, 1H), 1.94 (dd, J=9.4, 3.9 Hz, 1H), 1.86-1.81 (m, 4H), 1.63 (s, 1H). [M+H]+=833.7.

### Example 200: (R)-3-(4-(4-((2R,5S)-2,5-dimethyl-4-((7¹R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-2,6-difluorophenyl)piperidine-2,6-dione

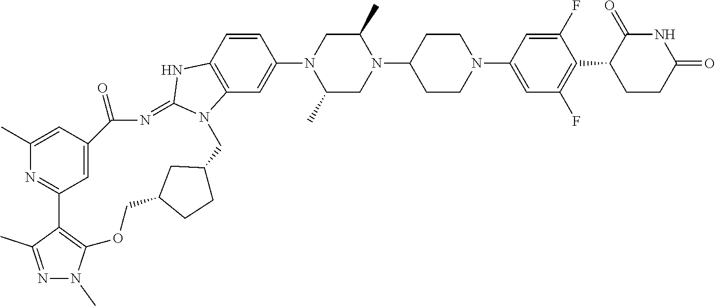 [SMILES: Cc1cc2cc(n1)-c1c(C)nn(C)c1OC[C@H]1CC[C@H](C1)CN1/C(=N/C2=O)Nc2ccc(N3C[C@@H](C)N(C4CCN(c5cc(F)c([C@H]6CCC(=O)NC6=O)c(F)c5)CC4)C[C@@H]3C)cc21]

The title compound was prepared in a manner similar to that in example 1 with intermediates 127 and 7. ¹H NMR (500 MHz, DMSO) δ 12.47 (s, 1H), 10.86 (s, 1H), 8.54 (s, 1H), 7.45 (s, 1H), 7.39 (d, J=8.6 Hz, 1H), 7.15 (s, 1H), 6.96 (d, J=8.3 Hz, 1H), 6.65 (s, 1H), 6.62 (s, 1H), 4.39-3.98 (m, 5H), 3.81 (d, J=10.7 Hz, 2H), 3.67 (s, 3H), 3.54-3.45 (m, 1H), 3.28 (s, 1H), 3.12-3.00 (m, 2H), 2.87-2.74 (m, 6H), 2.59-2.52 (m, 6H), 2.48 (s, 3H), 2.37-2.35 (m, 1H), 2.15-2.04 (m, 1H), 2.00-1.94 (m, 1H), 1.90-1.62 (m, 8H), 1.51-1.41 (m, 1H), 1.04 (d, J=5.9 Hz, 3H), 0.88 (d, J=6.0 Hz, 3H). [M+H]⁺=875.7.

### Example 202: (R)-3-(2,6-difluoro-4-(4-((S)-3-(fluoromethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

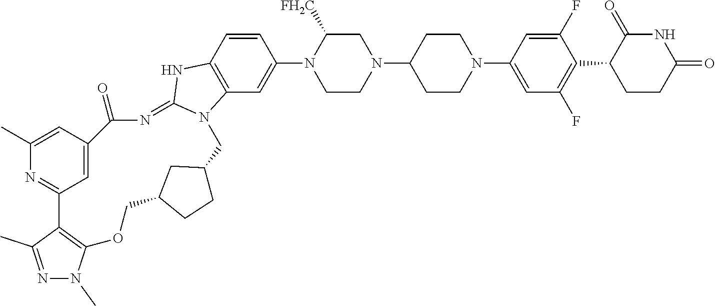

The title compound was prepared in a manner similar to that in example 1 with intermediates 128 and 7. ¹H NMR (500 MHz, DMSO) δ 12.39 (s, 1H), 10.93-10.79 (m, 1H), 8.53 (s, 1H), 7.48-7.42 (m, 1H), 7.39-7.33 (m, 1H), 7.05 (s, 1H), 6.93 (d, J=9.1 Hz, 1H), 6.66 (s, 1H), 6.63 (s, 1H), 4.74-4.58 (m, 1H), 4.45-4.33 (m, 1H), 4.32-4.24 (m, 1H), 4.23-3.99 (m, 5H), 3.80 (d, J=11.8 Hz, 2H), 3.66 (s, 3H), 3.36 (d, J=11.3 Hz, 1H), 3.28 (s, 1H), 3.12-2.91 (m, 3H), 2.84-2.74 (m, 3H), 2.62-2.52 (m, 8H), 2.48-2.39 (m, 5H), 2.15-2.04 (m, 1H), 2.00-1.93 (m, 1H), 1.91-1.75 (m, 5H), 1.68-1.58 (m, 1H), 1.56-1.45 (m, 2H). [M+H]+=879.7.

### Example 205: (R)-3-(4-(3-(((S)-2-(difluoromethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)methyl)azetidin-1-yl)-2,6-difluorophenyl)piperidine-2,6-dione

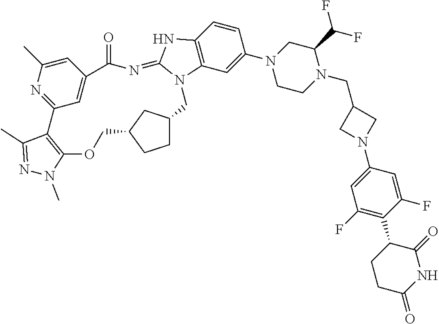 [SMILES: Cc1cc2cc(n1)-c1c(C)nn(C)c1OC[C@H]1CC[C@H](C1)CN1/C(=N/C2=O)Nc2ccc(N3CCN(CC4CN(c5cc(F)c([C@H]6CCC(=O)NC6=O)c(F)c5)C4)[C@H](C(F)F)C3)cc21]

The title compound was prepared in a manner similar to that in example 1 with intermediates 129 and 44. ¹H NMR (500 MHz, DMSO) δ 12.46 (s, 1H), 10.85 (s, 1H), 8.54 (s, 1H), 7.45 (s, 1H), 7.39 (d, J=8.7 Hz, 1H), 7.04 (s, 1H), 6.90 (d, J=8.6 Hz, 1H), 6.13 (s, 1H), 6.11 (s, 1H), 4.31 (s, 1H), 4.19 (s, 1H), 4.03 (dd, J=12.5, 4.9 Hz, 2H), 3.93 (t, J=7.4 Hz, 2H), 3.67 (s, 3H), 3.54-3.51 (m, 2H), 3.24 (s, 3H), 3.11 (d, J=10.8 Hz, 2H), 3.08-3.02 (m, 3H), 3.00-2.96 (m, 1H), 2.88-2.84 (m, 1H), 2.79-2.76 (m, 2H), 2.69-2.63 (m, 2H), 2.56-2.55 (m, 6H), 2.48 (s, 4H), 2.11-2.04 (m, 1H), 1.96-1.92 (m, 1H), 1.82 (s, 3H), 1.64 (s, 1H). [M+H]+=883.6.

### Example 206: (R)-3-(2,6-difluoro-4-(4-(3-((R)-2-(methoxymethyl)-4-((7R,7³S,E)-1¹,1³2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)azetidin-1-yl)piperidin-1-yl)phenyl)piperidine-2,6-dione

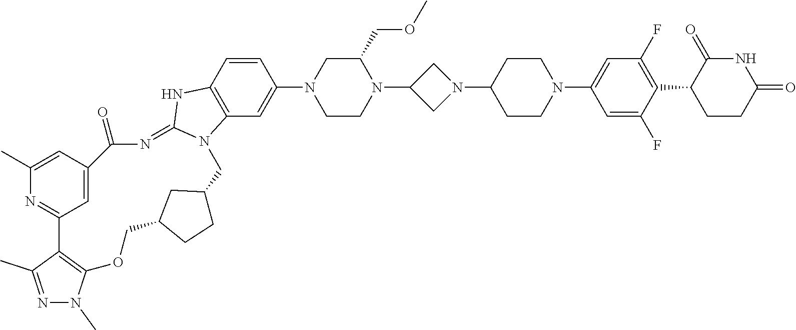 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CN(C2CCN(c3cc(F)c([C@H]4CCC(=O)NC4=O)c(F)c3)CC2)C1]

The title compound was prepared in a manner similar to that in example 1 with intermediates 130 and 7. ¹H NMR (500 MHz, DMSO) δ 12.41 (s, 1H), 10.87 (s, 1H), 8.53 (s, 1H), 7.45 (s, 1H), 7.37 (d, J=8.7 Hz, 1H), 6.98 (s, 1H), 6.87 (d, J=8.8 Hz, 1H), 6.61 (d, J=12.9 Hz, 2H), 4.29-4.01 (m, 6H), 3.67 (s, 2H), 3.63-3.55 (m, 3H), 3.50-3.39 (m, 5H), 3.28-3.18 (m, 5H), 3.12-2.99 (m, 2H), 2.88-2.73 (m, 7H), 2.69-2.62 (m, 3H), 2.55 (s, 3H), 2.48 (s, 3H), 2.37-2.34 (m, 1H), 2.23-2.16 (m, 1H), 2.11-2.01 (m, 1H), 1.99-1.93 (m, 1H), 1.90-1.76 (m, 4H), 1.69-1.67 (m, 3H), 1.22-1.21 (m, 2H). [M+H]⁺=946.5.

### Example 207: 3-((4-(4-((R)-2-(methoxymethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)piperazin-1-yl)piperidin-1-yl)-3-methylphenyl)amino)piperidine-2,6-dione

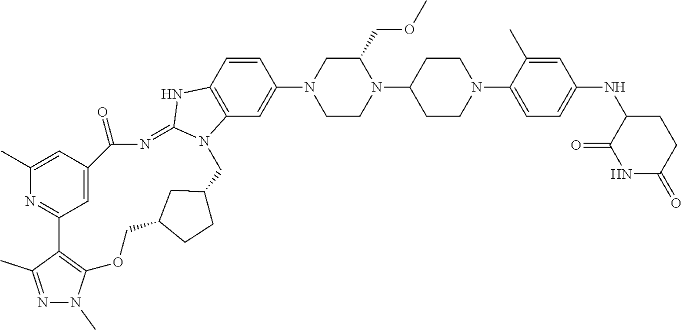 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2ccc(NC3CCC(=O)NC3=O)cc2C)CC1]

The title compound was prepared in a manner similar to that in example 1 with intermediates 47 and 70. ¹H NMR (500 MHz, DMSO) δ 12.32 (s, 1H), 10.75 (s, 1H), 8.54 (s, 1H), 7.45 (s, 1H), 7.37 (d, J=8.7 Hz, 1H), 6.99 (s, 1H), 6.88 (d, J=8.7 Hz, 1H), 6.83 (d, J=8.6 Hz, 1H), 6.52 (d, J=2.5 Hz, 1H), 6.46 (dd, J=8.6, 2.4 Hz, 1H), 5.44 (d, J=7.5 Hz, 1H), 4.44-3.96 (m, 5H), 3.67 (s, 3H), 3.58-3.49 (m, 2H), 3.31-3.27 (m, 7H), 3.06-2.92 (m, 5H), 2.90-2.86 (m, 1H), 2.80-2.68 (m, 3H), 2.63-2.58 (m, 3H), 2.53-2.51 (m, 2H), 2.55 (s, 3H), 2.48 (s, 3H), 2.16 (s, 3H), 2.13-2.07 (m, 1H), 1.93-1.80 (m, 5H), 1.75-1.71 (m, 2H), 1.55-1.53 (m, 2H). [M+H]⁺=884.7.

### Example 208: (R)-3-(4-(4-(3-((R)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-(methoxymethyl)piperazin-1-yl)azetidin-1-yl)piperidin-1-yl)-2,6-difluorophenyl)piperidine-2,6-dione

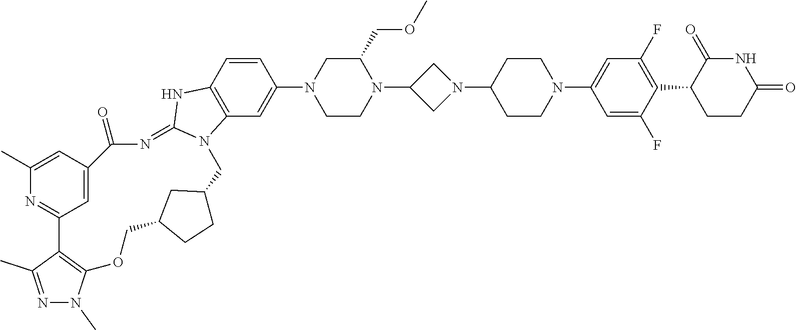 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CN(C2CCN(c3cc(F)c([C@H]4CCC(=O)NC4=O)c(F)c3)CC2)C1]

The title compound was prepared in a manner similar to that in example 1 with intermediates 131 and 7. ¹H NMR (500 MHz, DMSO) δ 12.42 (s, 1H), 10.86 (s, 1H), 8.52 (s, 1H), 7.89 (s, 1H), 7.45 (s, 1H), 7.37 (d, J=8.7 Hz, 1H), 6.98 (s, 1H), 6.87 (d, J=9.0 Hz, 1H), 6.61 (d, J=12.9 Hz, 2H), 4.33-4.30 (m, 1H), 4.20-4.17 (m, 1H), 4.13-3.94 (m, 4H), 3.74 (s, 3H), 3.60-3.57 (m, 3H), 3.48-3.40 (m, 6H), 3.27 (s, 3H), 3.26-3.21 (m, 4H), 3.09-3.01 (m, 3H), 2.92-2.77 (m, 4H), 2.69-2.63 (m, 5H), 2.55 (s, 3H), 2.20-2.16 (m, 1H), 2.10-2.06 (m, 1H), 1.97-1.93 (m, 1H), 1.84-1.80 (m, 2H), 1.70-1.66 (m, 2H), 1.23-1.21 (m, 2H). [M+H]⁺=932.5.

### Example 210: (R)-3-(2,6-difluoro-4-(3-(((R)-2-(fluoromethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)methyl)azetidin-1-yl)phenyl)piperidine-2,6-dione

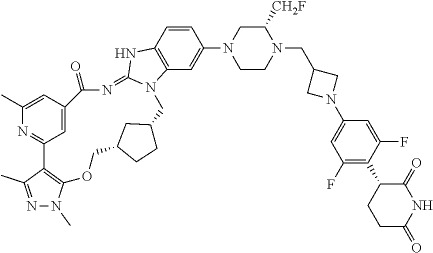

The title compound was prepared in a manner similar to that in example 1 with intermediates 132 and 44. ¹H NMR (500 MHz, DMSO) δ 12.40 (s, 1H), 10.84 (s, 1H), 8.54 (s, 1H), 7.45 (s, 1H), 7.38 (d, J=8.7 Hz, 1H), 7.04 (s, 1H), 6.91 (d, J=7.5 Hz, 1H), 6.13 (s, 1H), 6.11 (s, 1H), 4.78-4.69 (m, 1H), 4.68-4.58 (m, 1H), 4.35-3.91 (m, 7H), 3.67 (s, 3H), 3.57-3.49 (m, 2H), 3.42 (d, J=10.0 Hz, 1H), 3.32 (s, 2H), 3.10-2.73 (m, 8H), 2.70-2.53 (m, 7H), 2.48-2.41 (m, 4H), 2.13-2.02 (m, 1H), 1.97-1.76 (m, 4H), 1.69-1.52 (m, 1H). [M+H]⁺=865.7.

### Example 211: (R)-3-(2,6-difluoro-4-(4-((R)-3-(fluoromethyl)-4-((7R,7³S,E)-1¹,1³,2⁶-trimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)- cyclopentanacyclononaphane-5⁶-yl)piperazin-1-yl)piperidin-1-yl)phenyl)piperidine-2,6-dione

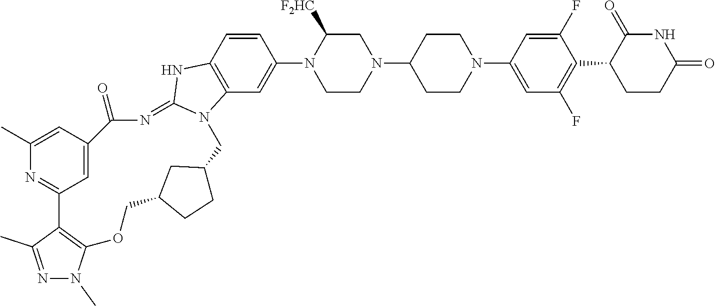 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C[C@@H]3C(F)F)cc21]

The title compound was prepared in a manner similar to that in example 1 with intermediates 133 and 7. ¹H NMR (500 MHz, DMSO) δ 12.40 (s, 1H), 10.86 (s, 1H), 8.54 (s, 1H), 7.45 (s, 1H), 7.36 (d, J=8.7 Hz, 1H), 7.05 (s, 1H), 6.92 (d, J=7.7 Hz, 1H), 6.65 (s, 1H), 6.63 (s, 1H), 4.74-4.58 (m, 1H), 4.45-4.25 (m, 2H), 4.23-4.15 (m, 2H), 4.12-3.95 (m, 3H), 3.80 (d, J=9.6 Hz, 2H), 3.67 (s, 3H), 3.41-3.31 (m, 2H), 3.10-2.91 (m, 3H), 2.83-2.74 (m, 3H), 2.65-2.52 (m, 8H), 2.48-2.38 (m, 5H), 2.14-2.03 (m, 1H), 1.99-1.93 (m, 1H), 1.90-1.77 (m, 5H), 1.70-1.58 (m, 1H), 1.56-1.42 (m, 2H). [M+H]⁺=879.7.

### Example 213: (R)-3-(2,6-difluoro-4-((1′-((7¹R,7³S,E)-1¹,1³,2⁶-trimethyl-3-oxo-5²,5³-dihydro-1¹H,1^{H}-9-oxa-4-aza-5(2,1)-benzo[d]imidazola-2(2,4)-pyridina-1(4,5)-pyrazola-7(1,3)-cyclopentanacyclononaphane-5⁶-yl)-[1,4′-bipiperidin]-4-yl)amino)phenyl)piperidine-2,6-dione

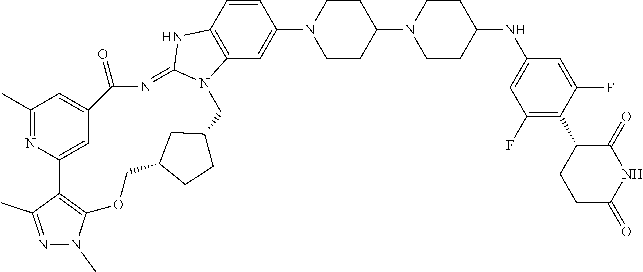 [SMILES: Cc1cc2cc(n1)-c1c(C)nn(C)c1OC[C@H]1CC[C@H](C1)CN1/C(=N/C2=O)Nc2ccc(N3CCC(N4CCC(Nc5cc(F)c([C@H]6CCC(=O)NC6=O)c(F)c5)CC4)CC3)cc21]

The racemate compound was prepared in a manner similar to that in example 1 with intermediates 101 and 134.

The title product was purified by chiral HPLC and corresponds to peak A @ 4.377 min.

Inj. Volume: 3.0 μL

Column: CHIRALPAK IF 4.6*150 mm 5 μm

Flow Rate (mL/min): 1.0

Gradient:

| Time (min) | MtBE (0.1% FA + 0.1% DEA)(%) | MeOH:DCM = 50:50(%) |
|---|---|---|
| 0 | 30 | 70 |
| 15 | 30 | 70 |

¹H NMR (500 MHz, DMSO) δ 12.42 (s, 1H), 10.82 (s, 1H), 8.54 (s, 1H), 7.45 (s, 1H), 7.36 (d, J=8.7 Hz, 1H), 7.03 (s, 1H), 6.92 (d, J=8.8 Hz, 1H), 6.24 (d, J=12.0 Hz, 2H), 6.14 (d, J=7.2 Hz, 1H), 4.39-3.91 (m, 5H), 3.77-3.72 (m, 2H), 3.67 (s, 3H), 3.25-3.22 (m, 6H), 2.95-2.88 (m, 2H), 2.83-2.62 (m, 5H), 2.55 (s, 3H), 2.48 (s, 3H), 2.38-2.35 (m, 2H), 2.11-2.01 (m, 1H), 1.93-1.88 (m, 8H), 1.66-1.60 (m, 2H), 1.38-1.36 (m, 2H). [M+H]⁺=861.4.

### Example 214: 3-(5-(4-((S)-4-((7¹R,7³S,E)-1¹,2⁶-dimethyl-3-oxo-5²,5³-dihydro-1¹H,5¹H-9-oxa-4-aza-5(2,1)-benzo[d]imidazola-2(2,4)-pyridina-1(4,5)-pyrazola-7(1,3)-cyclopentanacyclononaphane-5⁶-yl)-2-methylpiperazin-1-yl)piperidin-1-yl)-6-methylpyridin-2-yl)piperidine-2,6-dione

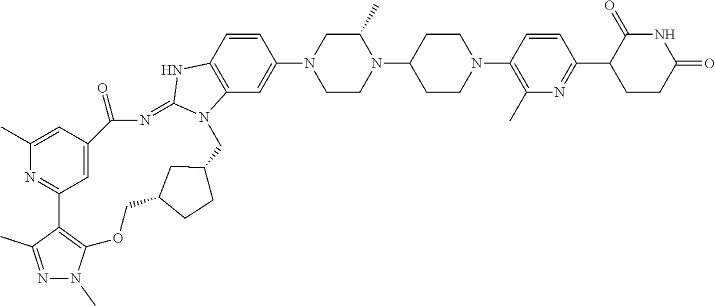 [SMILES: Cc1cc2cc(n1)-c1c(C)nn(C)c1OC[C@H]1CC[C@H](C1)CN1/C(=N/C2=O)Nc2ccc(N3CCN(C4CCN(c5ccc(C6CCC(=O)NC6=O)nc5C)CC4)[C@@H](C)C3)cc21]

The title compound was prepared in a manner similar to that in example 1 with intermediates 71 and 14. ¹H NMR (500 MHz, DMSO) δ 12.42 (s, 1H), 10.78 (s, 1H), 8.53 (s, 1H), 7.89 (s, 1H), 7.45 (s, 1H), 7.37 (t, J=8.2 Hz, 2H), 7.11 (d, J=8.2 Hz, 1H), 7.01 (s, 1H), 6.92 (d, J=8.6 Hz, 1H), 4.30 (s, 1H), 4.19 (s, 1H), 4.10 (s, 1H), 3.89 (dd, J=9.5, 5.3 Hz, 1H), 3.74 (s, 3H), 3.45 (d, J=9.5 Hz, 2H), 3.15 (d, J=9.4 Hz, 3H), 2.95-2.88 (m, 5H), 2.72 (t, J=11.8 Hz, 1H), 2.63 (s, 3H), 2.61-2.59 (m, 2H), 2.58-2.56 (m, 2H), 2.55 (s, 3H), 2.54-2.52 (m, 1H), 2.41 (s, 3H), 2.24-2.18 (m, 1H), 2.12-2.07 (m, 1H), 1.97-1.90 (m, 1H), 1.85-1.81 (m, 4H), 1.73-1.68 (m, 2H), 1.58 (d, J=8.6 Hz, 1H), 1.14 (d, J=6.2 Hz, 3H). [M+H]⁺=826.7.

### Cell Line

H1975-clone #8 (L858R/C797S): EGFR-L858R/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/cell, cultured for about 2 weeks to allow single clones formation. The formed clones were screened by DNA sequencing and whole exon sequencing analysis for the desired edition. H1975-clone #8 were finally confirmed as homozygous L858R/C797S EGFR clones.

H1975-clone #23 (Del19/C797S): EGFR-Del19/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by the knockout, the edited H1975 cells were seeded in 96 well plates at the concentration of 1 cell/well, cultured for about 2 weeks to allow single clones formation. The formed clones were screened by DNA sequencing and whole exon sequencing analysis for the desired edition. H1975-clone #23 were finally confirmed as homozygous Del19/C797S EGFR clones.

BaF3-L858R (abbv. L858R) cell were purchased from Kangyuan Bochuang Biotechnology (Beijing) Co., Ltd.

### Cell Degradation

### Cell Treatment

On day 1, H1975-clone #8 (L858R/C797S) cells are seeded at 5000 cells/well in cell culture medium [RPMI1640 (Gibco, Cat #72400-047), 10% heat-inactive FBS, 1% PS (Gibco, Cat #10378)] in Corning 96 well plate (Cat #3599).

On day 2, BaF3-L858R cells are seeded at 50000 cells/well at a volume of 54 μl/well in cell culture medium [RPMI1640 (Gibco, phenol red free, Cat #11835-030), 10% heat-inactive FBS, 1% PS (Gibco, Cat #10378)] in Corning 96 well plate (Cat #3799).

H1975-#8 and BaF3-L858R cells are treated with compounds diluted in 0.1% DMSO cell culture medium on day 2, incubate for 16 h, 37° C., 5% CO₂. the final concentration of compounds in all assay is start with 10 uM, 5-fold dilution, total 8 doses were included.

### HTRF Assay

After 16 h treatment, for H1975-#8 cells, add 100 ul HTRF 1× lysis buffer to each well; for BaF3-L858R cells, add 20 μl 4× lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group

Low control=only lysis buffer without cells, indicating that EGFR is completely degraded;

High control=Cell group with added DMSO and without compound, indicating microplate readings without EGFR degradation;

Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

Cell p-EGFR Inhibition Assay

### Cell Treatment

On day 1, H1975-clone #23 (DEL19/C797S) cells are seeded at 3×10⁴ cells/well in cell culture medium [RPMI1640 (Gibco, Cat #72400-047), 10% heat-inactive FBS, 1% PS (Gibco, Cat #10378)] in Corning 96 well plate (Cat #3599).

On day 2, BaF3-L858R cells are seeded at 2×10⁵ cells/well at a volume of 54 μl/well in cell culture medium [RPMI1640 (Gibco, phenol red free, Cat #11835-030), 10% heat-inactive FBS, 1% PS (Gibco, Cat #10378)] in Corning 96 well plate (Cat #3799).

H1975-#23 and BaF3-L858R cells are treated with compounds diluted in 0.1% DMSO cell culture medium on day 2, incubate for 16 h, 37° C., 5% CO₂. the final concentration of compounds in all assay is start with 10 uM, 5-fold dilution, total 8 doses were included.

### HTRF Assay

After 16 h treatment, for H1975-#23 cells, add 50 ul HTRF 1× lysis buffer to each well; for BaF3-L858R cells, add 20 μl 4× lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition percentage of the compound was calculated by the following equation: Inhibition percentage of Compound=100−100×(Signal−low control)/(High control−low control), wherein signal=each test compound group

Low control=only lysis buffer without cells, indicating that p-EGFR is completely inhibited.

High control=Cell group with added DMSO and without compound, indicating microplate readings without p-EGFR inhibited.

Imax is the maximum percentage of inhibition.

The IC₅₀ value of a compound can be obtained by fitting the following equation.

$$Y = Bottom + ( TOP - Bottom ) / ( 1 + ( ( {IC}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (BaF3-L858R and H1975-clone#8 (L858R/C797S))
result for Examples

| | BaF3-L858R | | H1975-clone#8 (L858R/C797S) | |
|---|---|---|---|---|
| Example No | DC₅₀ (nM) | Dₘₐₓ (%) | DC₅₀ (nM) | Dₘₐₓ (%) |
| 1 | 3.25 | 89.7 | 1.41 | 79.19 |
| 2 | 0.879 | 87.15 | 1.38 | 72.6 |
| 3 | 20.09 | 72.72 | 16.35 | 43.95 |
| 4 | 1.23 | 87.59 | 2.34 | 75.42 |
| 5 | 1.01 | 84.37 | 0.856 | 75.68 |
| 6 | 3.1 | 100.63 | 1.2 | 86.64 |
| 7 | 0.631 | 99.39 | 2.0316 | 45.4 |
| 8 | 3.2 | 64.04 | 3.18 | 74.17 |
| 9 | 6.11 | 86.6 | 4.28 | 71.65 |
| 10 | 4.07 | 73.02 | 3.15 | 66.8 |
| 11 | 0.834 | 84.59 | 3.75 | 61.52 |
| 12 | 0.493 | 90 | 0.774 | 88.41 |
| 13 | 0.307 | 91.5 | 1.4 | 65.47 |
| 14 | 0.552 | 83.99 | 0.616 | 90.93 |
| 15 | 7.6 | 94.19 | 11.11 | 85.15 |
| 21 | 2.99 | 98.96 | 0.782 | 83.18 |
| 23 | 12.82 | 86.05 | 0.608 | 54.98 |
| 27 | 1.75 | 91.12 | 3.19 | 90.21 |
| 35 | 0.49 | 91.75 | 0.6 | 58.02 |
| 57 | 3.84 | 82.6 | 3.14 | 58.49 |
| 59 | 3.18 | 85.1 | 2.87 | 68.95 |
| 63 | 0.55 | 90.04 | 0.61 | 60.89 |
| 64 | 1.07 | 83.31 | 1.87 | 69.97 |
| 65 | 0.94 | 88.69 | 0.87 | 78.56 |
| 69 | 3.2 | 89.35 | 5.24 | 62.39 |
| 70 | 3.78 | 87.66 | 4.11 | 73.46 |
| 71 | 3.63 | 84.61 | 3.3 | 80.16 |
| 72 | 0.4 | 91.61 | 0.64 | 76.57 |
| 73 | 2.02 | 87.22 | 2.24 | 78.37 |
| 74 | 0.99 | 90.43 | 1 | 86.22 |
| 75 | 1.6 | 82.72 | 0.97 | 61.33 |
| 76 | 3.23 | 84.83 | 3.87 | 79.39 |
| 77 | 3.4 | 89.47 | 5.5 | 85.66 |
| 78 | 3.68 | 87.78 | 4.18 | 65.49 |
| 79 | 0.41 | 92.61 | 0.48 | 91.69 |
| 80 | 1.73 | 88.11 | 5.24 | 82.36 |
| 82 | 0.31 | 74.12 | 0.8 | 89.18 |
| 83 | 4.08 | 69.13 | 5.64 | 81.8 |
| 84 | 4.01 | 79.65 | 2.1 | 64.66 |
| 85 | 4.23 | 77.98 | 4.91 | 82.96 |
| 86 | 2.74 | 70.33 | 3.22 | 65.99 |
| 87 | 2.86 | 64.11 | 8.71 | 70.8 |
| 88 | 16.41 | 95.81 | 1.63 | 82.92 |
| 89 | 1.75 | 73.86 | 1.64 | 87.47 |
| 90 | 3.21 | 80.57 | 2.06 | 87.4 |
| 91 | 2.04 | 81.49 | 2.28 | 88.66 |
| 92 | 1.97 | 85.77 | 3.12 | 79.85 |
| 93 | 9.09 | 77.38 | 7.66 | 83.77 |
| 94 | 3.37 | 71.57 | 0.83 | 73.9 |
| 95 | 8.5 | 86.55 | 12.13 | 78.96 |
| 96 | 3.41 | 86.61 | 3.15 | 82.66 |
| 98 | 2.6 | 80.59 | 2.49 | 75 |
| 99 | 1.06 | 82.32 | 6 | 85.62 |
| 100 | 3.34 | 72.3 | 3.07 | 87.33 |
| 101 | 2.22 | 81.78 | 1.58 | 90.21 |
| 102 | 0.59 | 85.75 | 0.76 | 91.52 |
| 103 | 3.38 | 81.62 | 2.53 | 83.31 |
| 104 | 4.1 | 83.17 | 3.7 | 86.92 |
| 105 | 3.68 | 82.74 | 4.09 | 85.25 |
| 106 | 6.69 | 82.51 | 2.45 | 83.27 |
| 108 | 3.32 | 87.31 | 0.75 | 76.98 |
| 111 | 3.18 | 80.74 | 1.92 | 85.67 |
| 112 | 2.2 | 72.83 | 0.41 | 67.55 |
| 113 | 3.49 | 70.53 | 2.69 | 77.35 |
| 114 | 3.11 | 75.67 | 1.09 | 81.96 |
| 115 | 1.6 | 81.91 | 2.84 | 81.08 |
| 116 | 1.25 | 85.38 | 0.61 | 83.42 |
| 117 | 1.11 | 78.04 | 0.92 | 80.56 |
| 118 | 2.64 | 79.03 | 5.97 | 73.74 |
| 119 | 2.47 | 77.02 | 2.3 | 74.58 |
| 120 | 3.14 | 70.84 | 0.55 | 65.65 |
| 121 | 1.06 | 82.19 | 0.98 | 88.32 |
| 122 | 4.53 | 70.68 | 3.24 | 52.22 |
| 123 | 3.37 | 77.06 | 2.14 | 81.58 |
| 124 | 3.58 | 71.14 | 2.3 | 67.53 |
| 125 | 1.37 | 77.11 | 2.61 | 67.41 |
| 126 | 2.04 | 83.28 | 1.49 | 73.72 |
| 127 | 9.26 | 88.84 | 6.07 | 81.87 |
| 129 | 16.21 | 71.91 | 1.45 | 69.35 |
| 131 | 4.84 | 69.11 | 0.867 | 72 |
| 132 | 3.22 | 81.02 | 2.05 | 65.95 |
| 133 | 2.36 | 85.73 | 2.94 | 86.47 |
| 134 | 1.89 | 85.87 | 4.97 | 79.89 |
| 135 | 3.08 | 89.06 | 1.91 | 86.89 |
| 136 | 3.52 | 81.06 | 0.152 | 76.08 |
| 137 | 2.37 | 83.34 | 3.35 | 78.23 |
| 138 | 3.61 | 85.49 | 1.27 | 83.42 |
| 140 | 3.04 | 93.28 | 1.94 | 85.59 |
| 141 | 1.13 | 87.5 | 0.804 | 66.08 |
| 142 | 7.26 | 80.29 | 0.748 | 87.35 |
| 143 | 5.23 | 74.32 | 1.51 | 81.95 |
| 144 | 3.1 | 73.64 | 0.938 | 84.77 |
| 147 | 1.04 | 87.63 | 2.02 | 63.2 |
| 150 | 8.77 | 90.48 | 7.29 | 84.98 |
| 151 | 1.68 | 90.59 | 2.62 | 88.52 |
| 152 | 1.19 | 86.64 | 1.28 | 88.69 |
| 153 | 2.8 | 84.4 | 2.92 | 86.96 |
| 156 | 2.11 | 87.41 | 2.73 | 87.98 |
| 157 | 2.03 | 85.83 | 1.88 | 87.96 |
| 158 | 3.75 | 79.81 | 4.39 | 77.26 |
| 159 | 9.56 | 77.55 | 3.75 | 81.28 |
| 160 | 3.08 | 76.5 | 3.01 | 88.21 |
| 161 | 7.2 | 91.43 | 2.96 | 89.3 |
| 162 | 4.86 | 88.6 | 3.14 | 85.43 |
| 163 | 4.4 | 88.11 | 2.97 | 82.53 |
| 164 | 2.79 | 92.71 | 1.19 | 88.71 |
| 165 | 3.32 | 91.08 | 4.45 | 88.72 |
| 167 | 4.72 | 83.25 | 4.33 | 88.56 |
| 168 | 2.96 | 80.9 | 3.07 | 81.06 |
| 169 | 2.62 | 86.46 | 2.81 | 91.64 |
| 172 | 5.09 | 84.83 | 0.909 | 85.56 |
| 173 | 4.28 | 95.16 | 4.31 | 91.39 |
| 175 | 4.07 | 88.67 | 7.25 | 83.91 |
| 176 | 8.27 | 89.05 | 10.66 | 85.37 |
| 179 | 5.38 | 90.75 | 10.57 | 89.58 |
| 182 | 21.77 | 89.15 | 1.39 | 79.84 |
| 183 | 5.3 | 91.58 | 5.83 | 91.15 |
| 184 | 4.99 | 90.13 | 3.16 | 89.28 |
| 186 | 3.82 | 80.04 | 2.92 | 80.4 |
| 188 | 4.2 | 93.16 | 3.78 | 84.89 |
| 189 | 4.4 | 87.56 | 2.38 | 90.22 |
| 190 | 3.11 | 88.2 | 1.69 | 94.92 |
| 191 | 2.94 | 89.33 | 0.73 | 88.66 |
| 192 | 2.42 | 87.87 | 1.01 | 88.8 |
| 193 | 7.4 | 76.16 | 0.76 | 70.78 |
| 195 | 1.83 | 90.93 | 1.13 | 93.91 |
| 196 | 1.61 | 91.87 | 3.21 | 88.79 |
| 197 | 8.79 | 73.99 | 7.24 | 89.7 |
| 198 | 7.56 | 89.74 | 4.41 | 100.08 |
| 199 | 6.02 | 82.45 | 2.28 | 91.82 |
| 200 | 2.1 | 85.73 | 1.87 | 94.58 |
| 202 | 1.06 | 92.5 | 4.8 | 87.91 |
| 205 | 5.75 | 90.35 | 3.05 | 85.78 |
| 206 | 1.1 | 92.06 | 0.54 | 90.68 |
| 207 | 1.04 | 91.91 | 1.7 | 87.5 |
| 208 | 0.67 | 91.62 | 1.01 | 70.65 |
| 210 | 0.74 | 90.25 | 3.72 | 86.86 |
| 211 | 0.73 | 91.28 | 3.07 | 88.78 |
| 213 | 2.18 | 88.29 | 0.63 | 82.54 |
| 214 | 0.65 | 82.61 | 0.61 | 77.32 |

TABLE 2
pEGFR inhibition (BaF3-L858R and H1975-clone#23 (DEL19/C797S))
result for Examples

| | | | H1975-clone#23 | |
|---|---|---|---|---|
| | BaF3-L858R | | (DEL19/C797S ) | |
| Example No | IC₅₀ (nM) | Iₘₐₓ (%) | IC₅₀ (nM) | Iₘₐₓ (%) |
| 35 | 1.3 | 91.6 | 0.5 | 101.1 |
| 57 | 10.5 | 101 | 1.2 | 97.9 |
| 59 | 7 | 102.5 | 0.7 | 99.6 |
| 63 | 0.2 | 103 | 0.2 | 100.6 |
| 64 | 3.2 | 101.9 | 0.7 | 101.2 |
| 65 | 2.8 | 103 | 0.3 | 101.5 |
| 69 | 5.7 | 99.9 | 1.8 | 100.1 |
| 70 | 11 | 102.1 | 1.4 | 99.2 |
| 71 | 6.4 | 99.7 | 1.1 | 99.8 |
| 72 | 3.2 | 92.8 | 0.6 | 101.7 |
| 73 | 10.2 | 93.5 | 1 | 101.1 |
| 74 | 3.2 | 93.3 | 1.7 | 101.2 |
| 75 | 7.9 | 92.6 | 0.6 | 101.1 |
| 76 | 7.8 | 94.9 | 0.7 | 101 |
| 77 | 10.9 | 91.3 | 9.5 | 99.2 |
| 78 | 16.9 | 92.5 | 1.2 | 100.6 |
| 79 | 1.1 | 92.7 | 0.3 | 101.4 |
| 80 | 3.8 | 91.7 | 1.6 | 99.9 |
| 82 | 3.1 | 91.9 | 1.1 | 101.1 |
| 83 | 18 | 96.5 | 1 | 100.7 |
| 84 | 6 | 109.3 | 0.5 | 100.5 |
| 85 | 11.6 | 97.5 | 3.1 | 99.7 |
| 86 | 17.2 | 97.1 | 3 | 100.2 |
| 87 | 13.8 | 96.2 | 1.5 | 100.3 |
| 88 | 6.1 | 108.3 | 3 | 100.7 |
| 89 | 8 | 99.7 | 1.9 | 101.2 |
| 90 | 6.3 | 98.4 | 2.5 | 101.7 |
| 91 | 3.5 | 97.1 | 2.2 | 100.8 |
| 92 | 10 | 97.2 | 1 | 100.1 |
| 93 | 19.6 | 96.9 | 2 | 100.3 |
| 94 | 6.1 | 99.6 | 0.8 | 101.2 |
| 95 | 10.8 | 99.3 | 4.5 | 101 |
| 96 | 6.3 | 100.3 | 0.8 | 100.9 |
| 98 | 4.7 | 110.3 | 1.4 | 101.3 |
| 99 | 8 | 100.3 | 2.2 | 99.2 |
| 100 | 6.7 | 107.3 | 0.5 | 101 |
| 101 | 3.5 | 106.4 | 1 | 101 |
| 102 | 1.5 | 95 | 0.6 | 100.5 |
| 103 | 16.3 | 95.6 | 0.8 | 102 |
| 104 | 4.4 | 95 | 2 | 100.5 |
| 105 | 10.1 | 93.9 | 0.7 | 100.1 |
| 106 | 16 | 97.6 | 0.8 | 99.8 |
| 108 | 7.7 | 95.9 | 0.6 | 100.3 |
| 111 | 16.1 | 97.6 | 1 | 100.1 |
| 112 | 15.4 | 104.4 | 0.4 | 98.9 |
| 113 | 17.1 | 94.6 | 0.7 | 99 |
| 114 | 7.3 | 98.4 | 0.4 | 100.5 |
| 115 | 3.9 | 97.7 | 1.3 | 99.8 |
| 116 | 7.9 | 98.5 | 0.8 | 99.4 |
| 117 | 14.9 | 97.1 | 0.6 | 100 |
| 118 | 7.3 | 98.5 | 1.3 | 100.2 |
| 119 | 18.2 | 94.3 | 0.6 | 100.8 |
| 120 | 15.6 | 94.2 | 1.1 | 99.1 |
| 121 | 6 | 96.6 | 0.7 | 100.8 |
| 122 | 11 | 96.8 | 0.5 | 98.9 |
| 123 | 16.3 | 95.3 | 0.7 | 100.4 |
| 124 | 17.1 | 94.6 | 0.9 | 99.6 |
| 125 | 20.65 | 98.6 | 2.2 | 101.9 |
| 126 | 13.3 | 99.16 | 1.44 | 102.92 |
| 127 | 4.89 | 101.7 | 4.06 | 100.85 |
| 131 | 6.4 | 102.8 | 0.796 | 103.08 |
| 132 | 62.72 | 97.32 | 0.63 | 100.62 |
| 133 | 2.88 | 96.88 | 1.45 | 99.55 |
| 134 | 2.49 | 98.27 | 1.76 | 92.81 |
| 135 | 2.32 | 104.33 | 1.4 | 104.78 |
| 136 | 8.91 | 104.73 | 1.44 | 103.49 |
| 137 | 14.62 | 96.47 | 0.528 | 98.8 |
| 138 | 7.09 | 104.27 | 1.37 | 102.96 |
| 140 | 6.86 | 96.47 | 1.03 | 101.57 |
| 141 | 7.7 | 98.22 | 0.825 | 100.38 |
| 142 | 4.77 | 97.8 | 1.42 | 102.03 |
| 143 | 7.8 | 98.2 | 1.15 | 101.28 |
| 144 | 5.5 | 98.7 | 0.97 | 100.78 |
| 150 | 72.06 | 91.27 | 2.16 | 100.12 |
| 151 | 5.03 | 95.95 | 1.02 | 99.54 |
| 152 | 15.03 | 95.61 | 2.46 | 100.06 |
| 153 | 11.1 | 97.42 | 1.56 | 100.23 |
| 156 | 4.81 | 95.16 | 0.789 | 100.92 |
| 157 | 2.87 | 96.89 | 1.1 | 100.52 |
| 158 | 72.63 | 95.45 | 3.65 | 100.46 |
| 160 | 3.02 | 94.19 | 0.59 | 95.87 |
| 161 | 4.51 | 93.23 | 0.49 | 95.04 |
| 162 | 3.68 | 93.37 | 0.21 | 95.16 |
| 163 | 2.24 | 94.53 | 0.6 | 96.05 |
| 164 | 1.85 | 94.91 | 0.254 | 96.95 |
| 165 | 4.83 | 93.3 | 1.34 | 94.62 |
| 167 | 6.86 | 98.09 | 3.01 | 102.84 |
| 168 | 8.76 | 100.64 | 2.56 | 102.22 |
| 169 | 8.76 | 100.12 | 2.5 | 102.95 |
| 172 | 6.2 | 100 | 2.74 | 103.44 |
| 173 | 11.51 | 98.39 | 3.94 | 103.01 |
| 175 | 2.83 | 98.51 | 1.28 | 100.72 |
| 176 | 15.22 | 94.16 | 3.6 | 101.78 |
| 179 | 14.4 | 99.14 | 2.85 | 100.7 |
| 182 | 2.2069 | 100.38 | 2.3787 | 99.73 |
| 183 | 5.3205 | 98.21 | 7.3233 | 99.41 |
| 184 | 1.2488 | 91.67 | 2.4765 | 100.52 |
| 186 | 4.7712 | 92 | 2.3748 | 100.56 |
| 188 | <0.1280 | 90.67 | 1.9541 | 99.79 |
| 189 | <0.1280 | 92.5 | 2.1098 | 100.03 |
| 190 | 0.58 | 91.5 | 1.1958 | 100.67 |
| 191 | 0.5282 | 90.84 | 0.7695 | 99.88 |
| 192 | 1.5333 | 89.34 | 1.3897 | 99.96 |
| 193 | 2.351 | 101.04 | 2.743 | 100.43 |
| 195 | <0.1280 | 90.34 | 1.4427 | 99.96 |
| 196 | 3.0921 | 85.84 | 2.8068 | 99.76 |
| 197 | 9.1633 | 101.23 | 3.3803 | 103.49 |
| 198 | 6.6611 | 101.37 | 3.1816 | 102.48 |
| 199 | <0.1280 | 99.05 | 2.9212 | 102.91 |
| 200 | <0.1280 | 98.42 | 1.7943 | 102.89 |
| 202 | 9.2508 | 92 | 2.4765 | 100.52 |
| 205 | 1.2176 | 92.5 | 0.5395 | 99.83 |
| 206 | 1.1741 | 94.08 | 0.8513 | 99.52 |
| 207 | 1.5018 | 88.5 | 1.3976 | 100.67 |
| 208 | 1.2132 | 91.5 | 3.0245 | 99.76 |
| 210 | 1.0438 | 89.17 | 1.5556 | 99.96 |
| 211 | 2.0494 | 89.34 | 1.5954 | 99.96 |
| 213 | 0.8707 | 88.33 | 1.6939 | 98.77 |
| 214 | 3.32 | 100.54 | 0.604 | 100.03 |

The foregoing examples and description of certain embodiments should be taken as illustrating, rather than as limiting, the present disclosure. As will be readily appreciated, numerous variations and combinations of the features set forth above can be utilized without departing from the present disclosure as set forth in the claims. All such variations are intended to be included within the scope of the present disclosure. All references cited are incorporated herein by reference in their entireties.

It is to be understood that, if any prior art publication is referred to herein, such reference does not constitute an admission that the publication forms a part of the common general knowledge in the art in any country.

1-76. (canceled)

77. A compound selected from 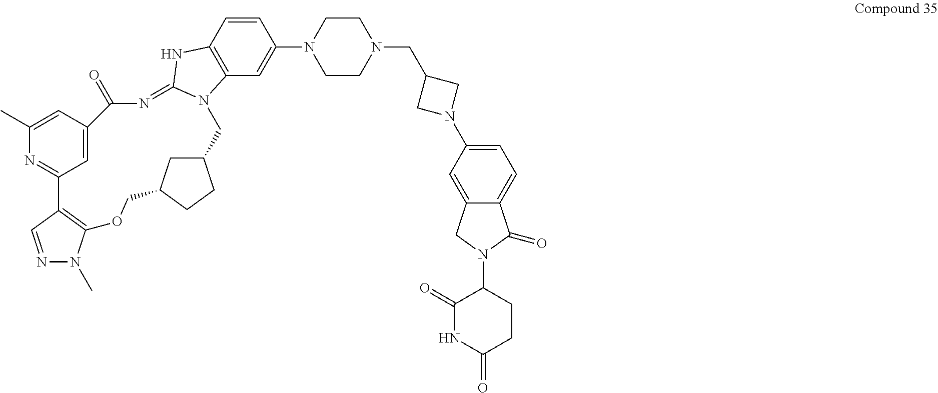 [SMILES: Cc1cc2cc(n1)-c1cnn(C)c1OC[C@H]1CC[C@H](C1)CN1/C(=N/C2=O)Nc2ccc(N3CCN(CC4CN(c5ccc6c(c5)CN(C5CCC(=O)NC5=O)C6=O)C4)CC3)cc21] 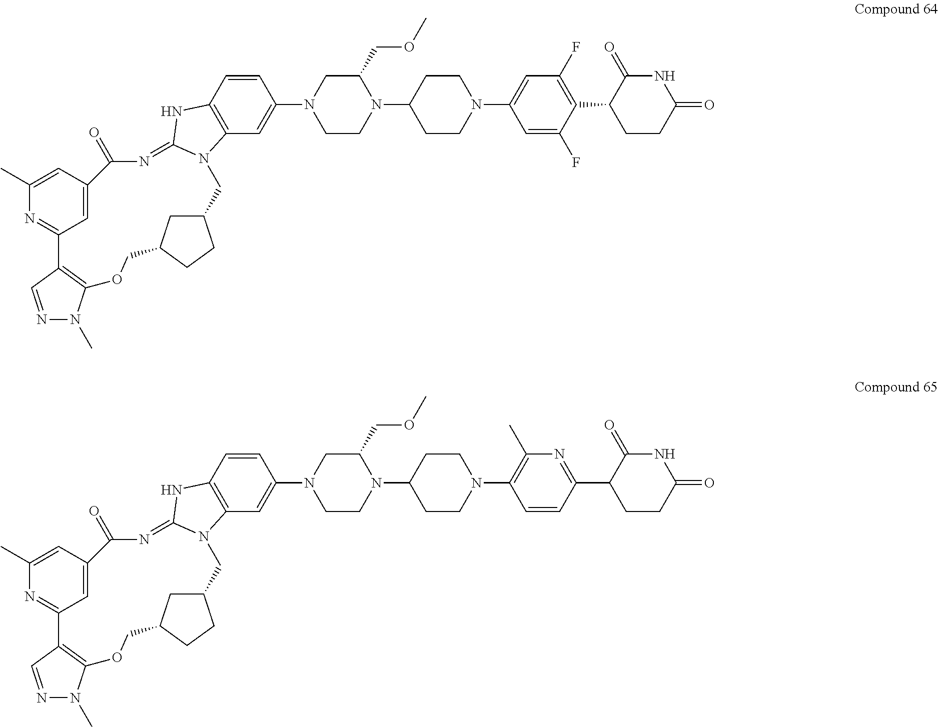 [SMILES: COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2cc(F)c([C@H]3CCC(=O)NC3=O)c(F)c2)CC1 ; COC[C@H]1CN(c2ccc3c(c2)N2C[C@@H]4CC[C@H](COc5c(cnn5C)-c5cc(cc(C)n5)C(=O)/N=C/2N3)C4)CCN1C1CCN(c2ccc(C3CCC(=O)NC3=O)nc2C)CC1] 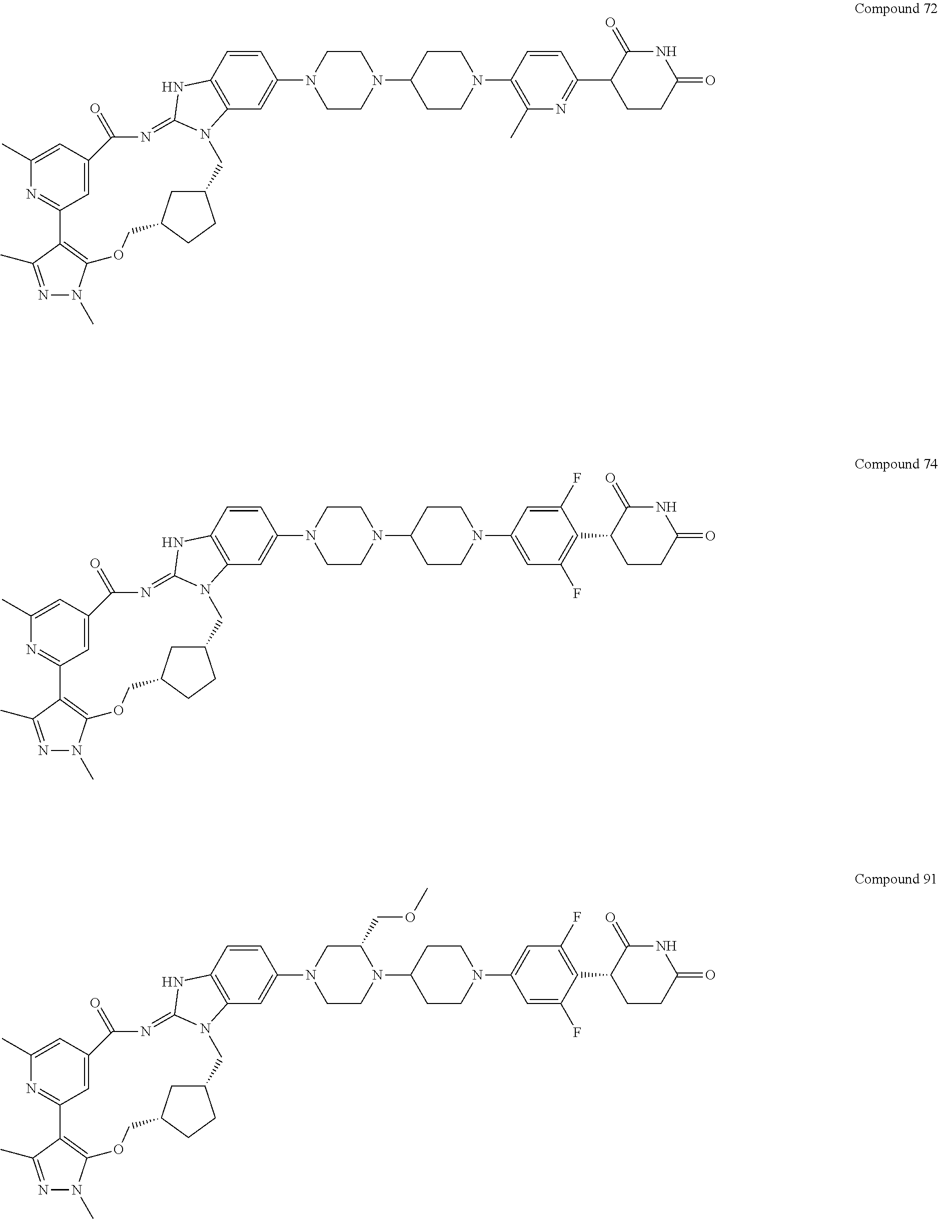 [SMILES: Cc1cc2cc(n1)-c1c(C)nn(C)c1OC[C@H]1CC[C@H](C1)CN1/C(=N/C2=O)Nc2ccc(N3CCN(C4CCN(c5ccc(C6CCC(=O)NC6=O)nc5C)CC4)CC3)cc21 ; Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)CC3)cc21 ; COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1] 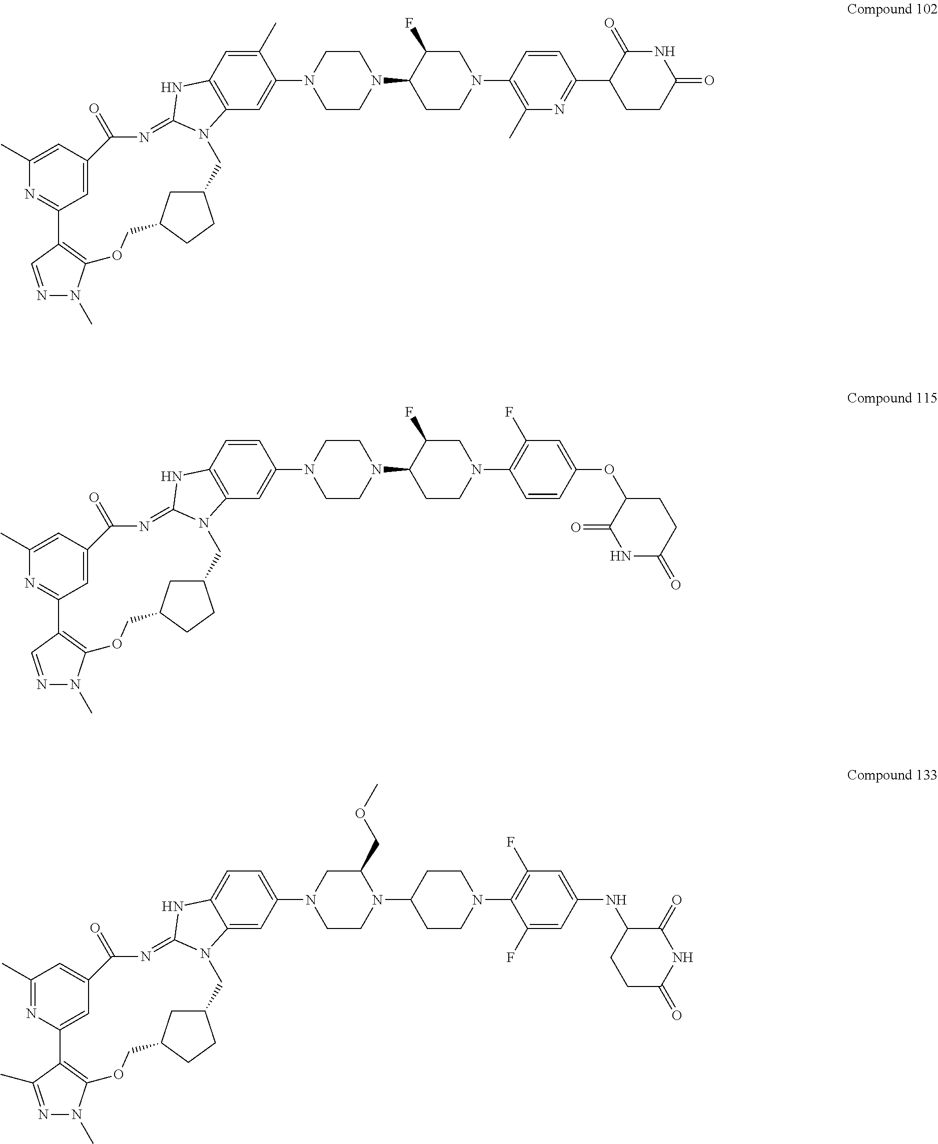 [SMILES: Cc1cc2cc(n1)-c1cnn(C)c1OC[C@H]1CC[C@H](C1)CN1/C(=N/C2=O)Nc2cc(C)c(N3CCN([C@@H]4CCN(c5ccc(C6CCC(=O)NC6=O)nc5C)C[C@@H]4F)CC3)cc21 ; Cc1cc2cc(n1)-c1cnn(C)c1OC[C@H]1CC[C@H](C1)CN1/C(=N/C2=O)Nc2ccc(N3CCN([C@@H]4CCN(c5ccc(OC6CCC(=O)NC6=O)cc5F)C[C@@H]4F)CC3)cc21 ; COC[C@@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2c(F)cc(NC3CCC(=O)NC3=O)cc2F)CC1] 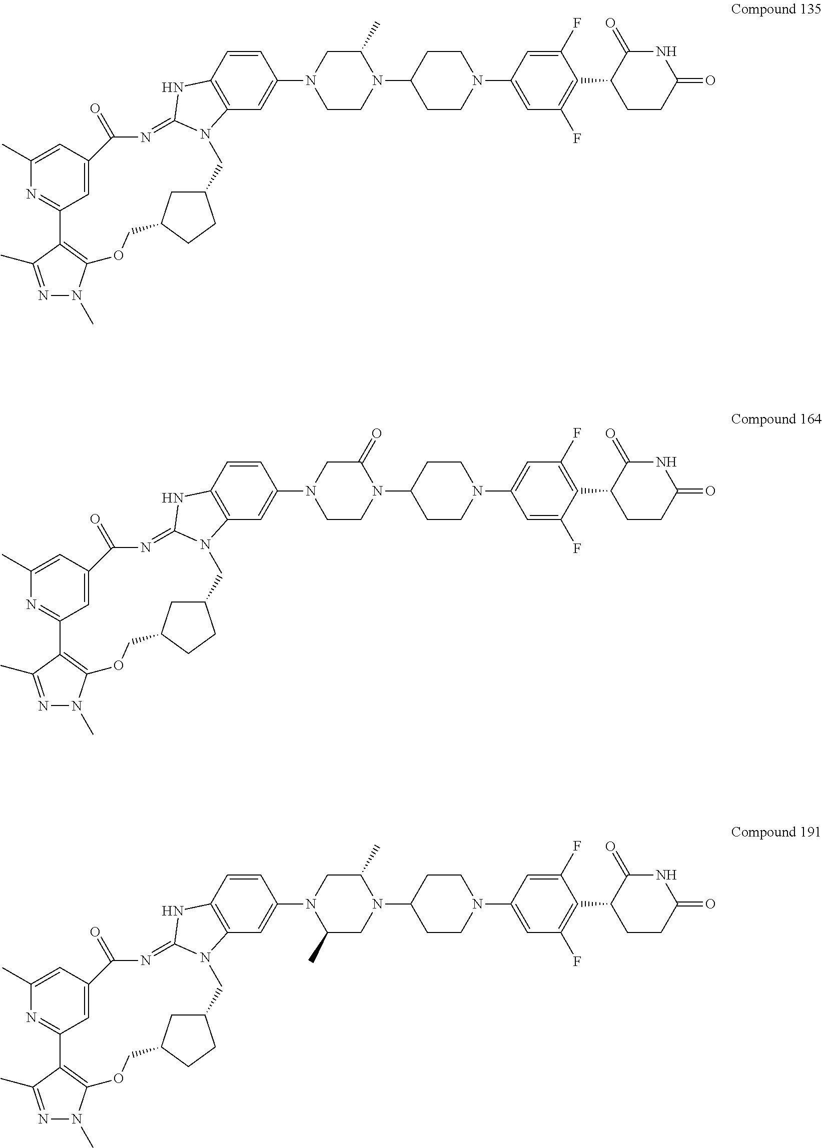 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)[C@@H](C)C3)cc21 ; Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)C(=O)C3)cc21 ; Cc1cc2cc(n1)-c1c(C)nn(C)c1OC[C@H]1CC[C@H](C1)CN1/C(=N/C2=O)Nc2ccc(N3C[C@H](C)N(C4CCN(c5cc(F)c([C@H]6CCC(=O)NC6=O)c(F)c5)CC4)C[C@H]3C)cc21] 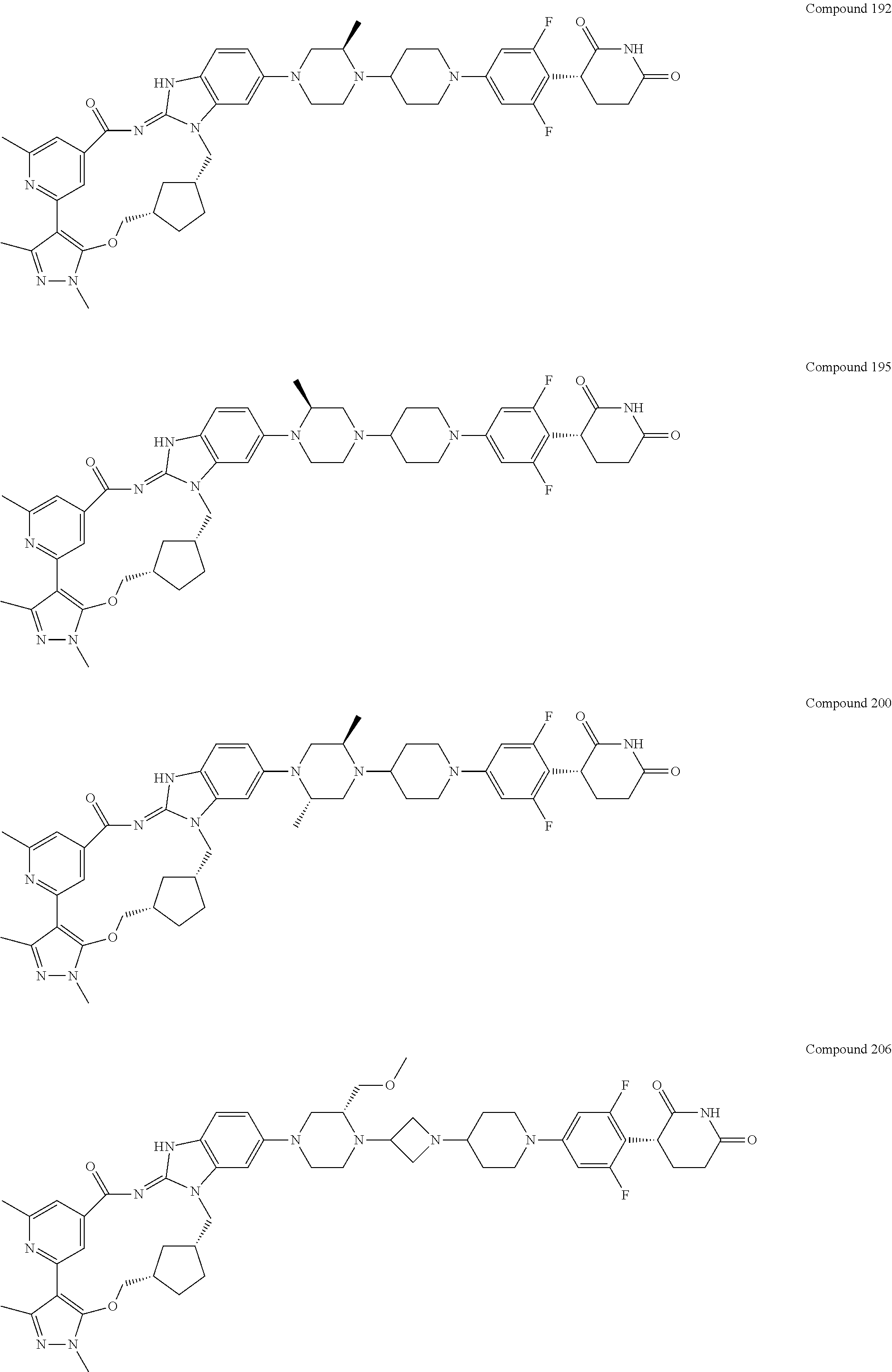 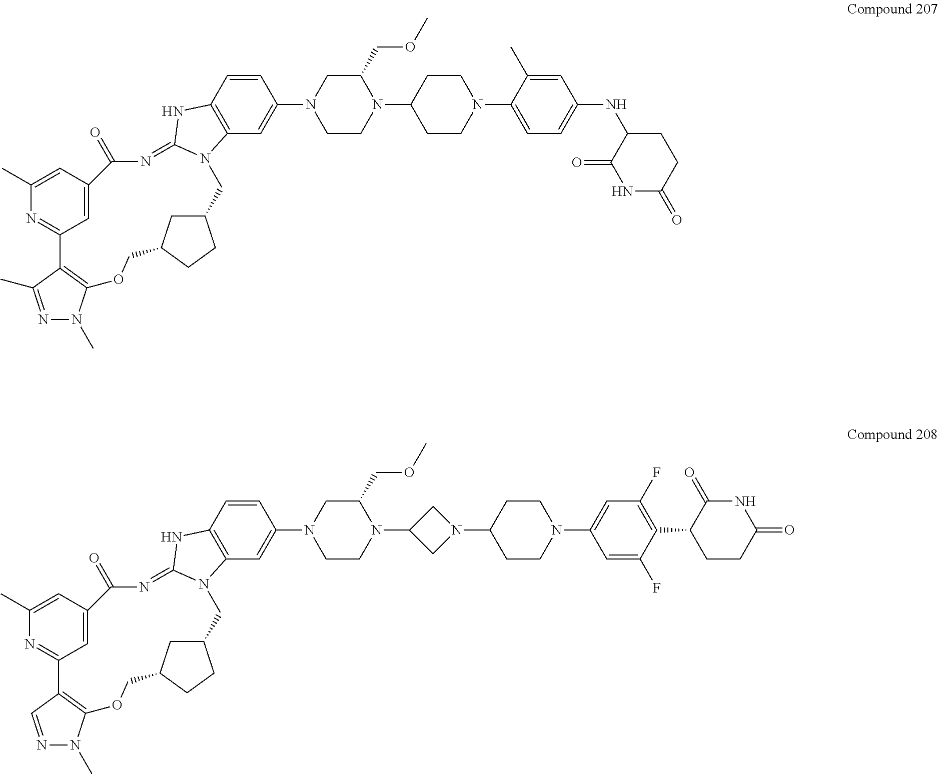 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2ccc(NC3CCC(=O)NC3=O)cc2C)CC1 ; COC[C@H]1CN(c2ccc3c(c2)N2C[C@@H]4CC[C@H](COc5c(cnn5C)-c5cc(cc(C)n5)C(=O)/N=C/2N3)C4)CCN1C1CN(C2CCN(c3cc(F)c([C@H]4CCC(=O)NC4=O)c(F)c3)CC2)C1] 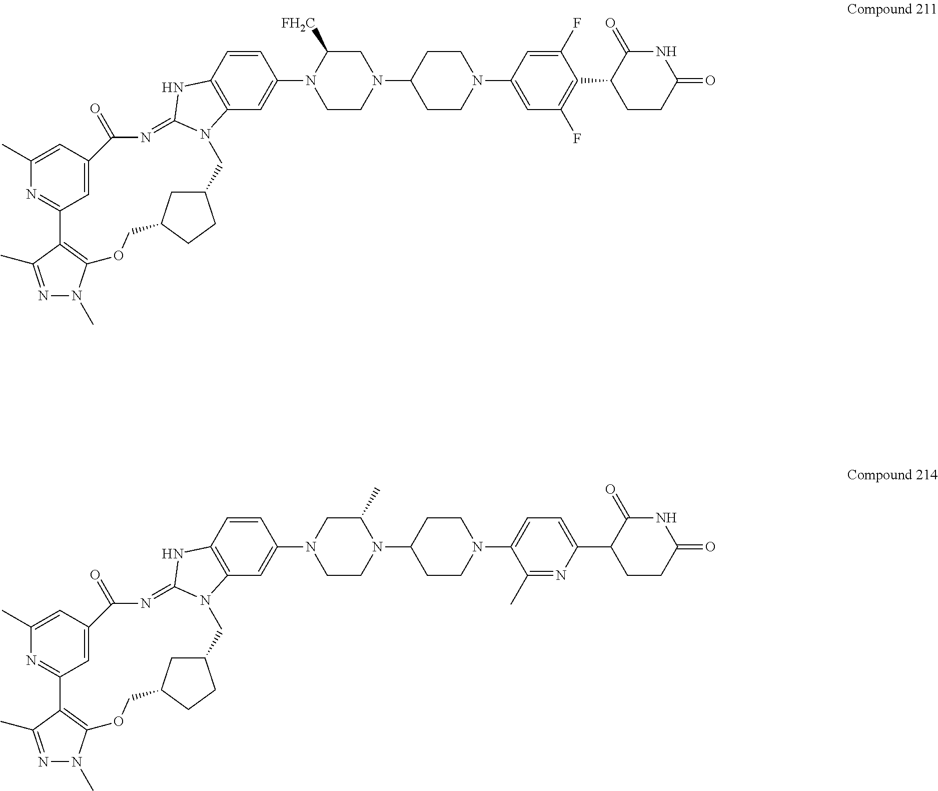
or a pharmaceutically acceptable salt thereof, or a stereoisomer thereof, or a tautomer thereof, or a deuterated analog thereof.

78. A pharmaceutical composition comprising a compound of claim 77 or a pharmaceutically acceptable salt thereof, a stereoisomer thereof, a tautomer thereof, or a deuterated analog, together with a pharmaceutically acceptable excipient.

79. A method of treating a disease that can be affected by EGFR modulation, comprises administrating a subject in need thereof an effective amount of a compound of claim 77 or a pharmaceutically acceptable salt thereof, a stereoisomer thereof, a tautomer thereof or a deuterated analog thereof.

80. The method of claim 79, wherein the disease is cancer.

81-82. (canceled)

83. The compound of claim 77, wherein the compound is

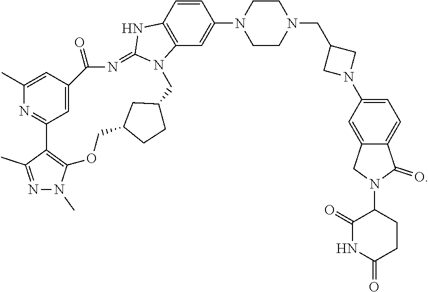 [SMILES: Cc1cc2cc(n1)-c1c(C)nn(C)c1OC[C@H]1CC[C@H](C1)CN1/C(=N/C2=O)Nc2ccc(N3CCN(CC4CN(c5ccc6c(c5)CN(C5CCC(=O)NC5=O)C6=O)C4)CC3)cc21]

84. The compound of claim 77, wherein the compound is

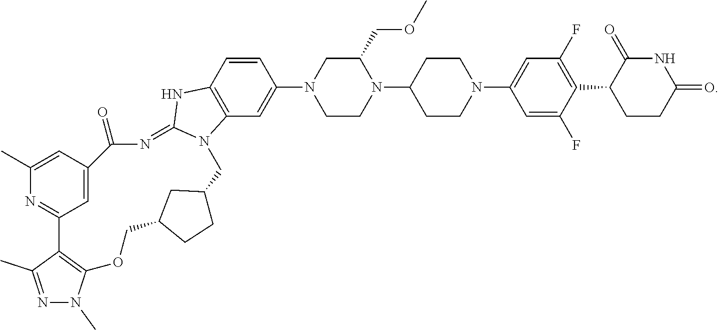 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2cc(F)c([C@H]3CCC(=O)NC3=O)c(F)c2)CC1]

85. The compound of claim 77, wherein the compound is

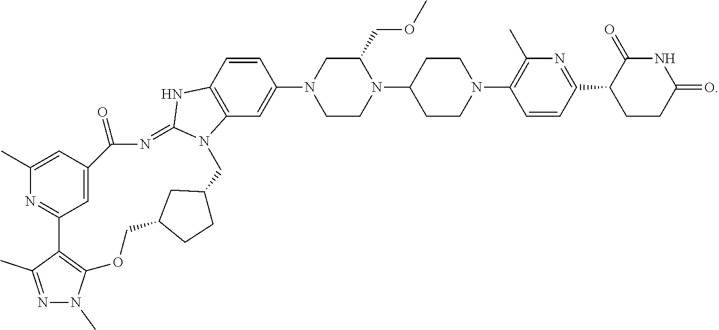 [SMILES: COC[C@H]1CN(c2ccc3c(c2)N2C[C@@H]4CC[C@H](COc5c(c(C)nn5C)-c5cc(cc(C)n5)C(=O)/N=C/2N3)C4)CCN1C1CCN(c2ccc([C@H]3CCC(=O)NC3=O)nc2C)CC1]

86. The compound of claim 77, wherein the compound is

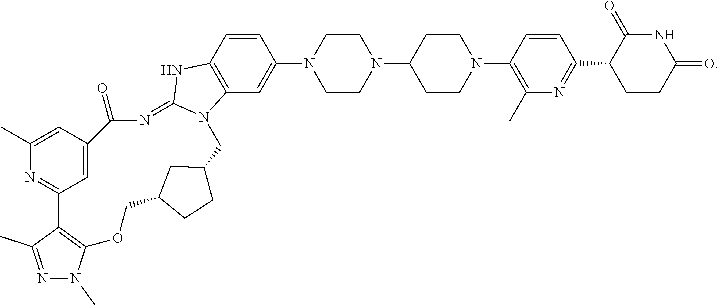 [SMILES: Cc1cc2cc(n1)-c1c(C)nn(C)c1OC[C@H]1CC[C@H](C1)CN1/C(=N/C2=O)Nc2ccc(N3CCN(C4CCN(c5ccc([C@H]6CCC(=O)NC6=O)nc5C)CC4)CC3)cc21]

87. The compound of claim 77, wherein the compound is

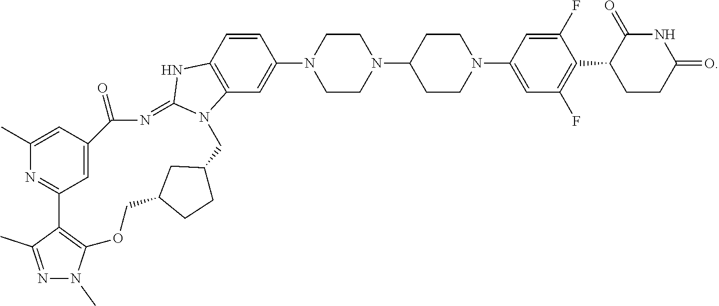 [SMILES: Cc1cc2cc(n1)-c1c(C)nn(C)c1OC[C@H]1CC[C@H](C1)CN1/C(=N/C2=O)Nc2ccc(N3CCN(C4CCN(c5cc(F)c([C@H]6CCC(=O)NC6=O)c(F)c5)CC4)CC3)cc21]

88. The compound of claim 77, wherein the compound is

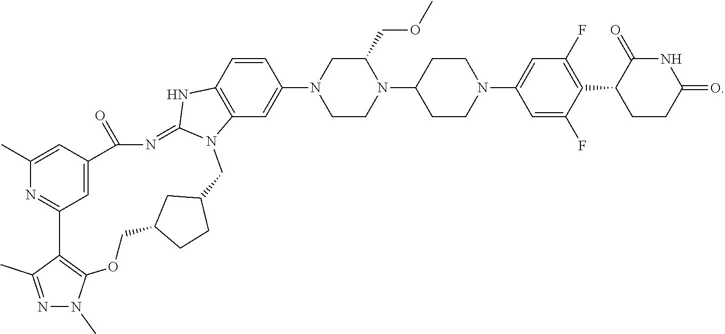

89. The compound of claim 77, wherein the compound is

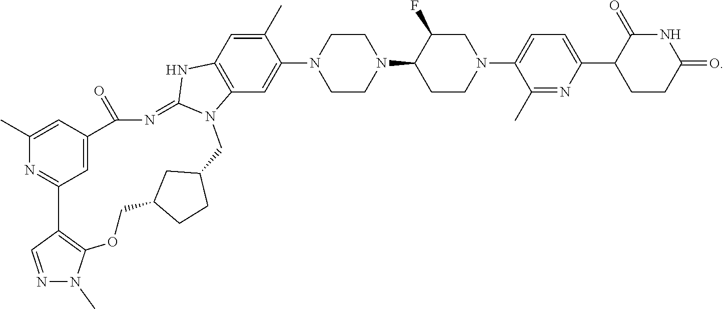 [SMILES: Cc1cc2cc(n1)-c1cnn(C)c1OC[C@H]1CC[C@H](C1)CN1/C(=N/C2=O)Nc2cc(C)c(N3CCN([C@@H]4CCN(c5ccc(C6CCC(=O)NC6=O)nc5C)C[C@@H]4F)CC3)cc21]

90. The compound of claim 77, wherein the compound is

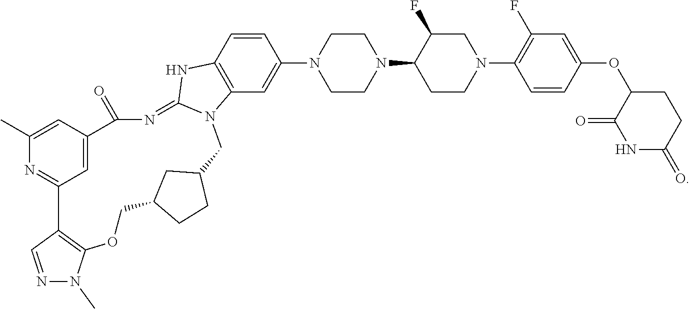

91. The compound of claim 77, wherein the compound is

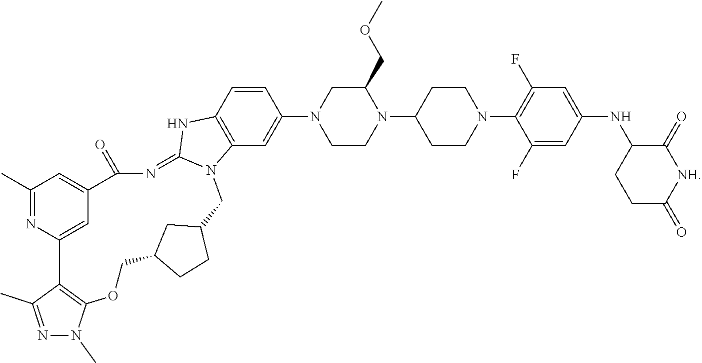

92. The compound of claim 77, wherein the compound is

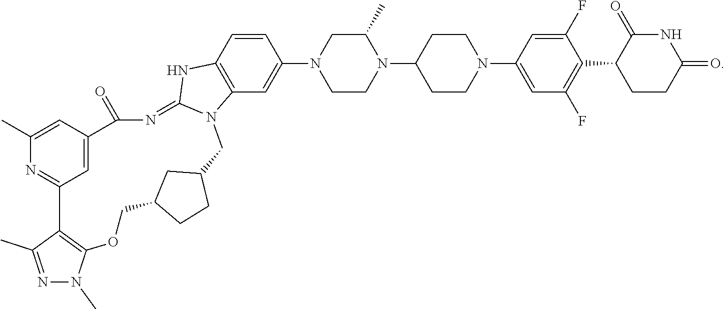

93. The compound of claim 77, wherein the compound is

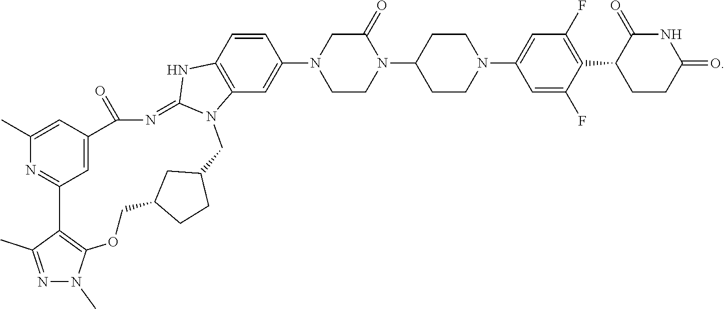

94. The compound of claim 77, wherein the compound is

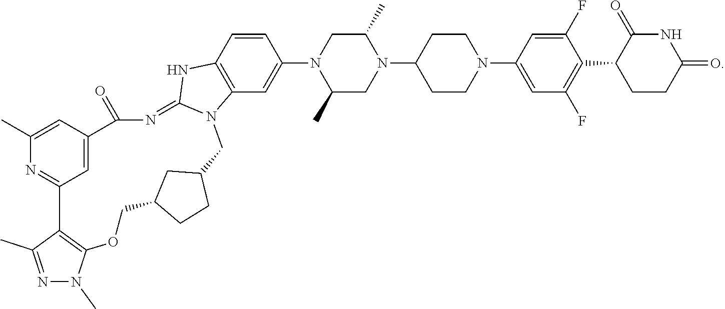

95. The compound of claim 77, wherein the compound is

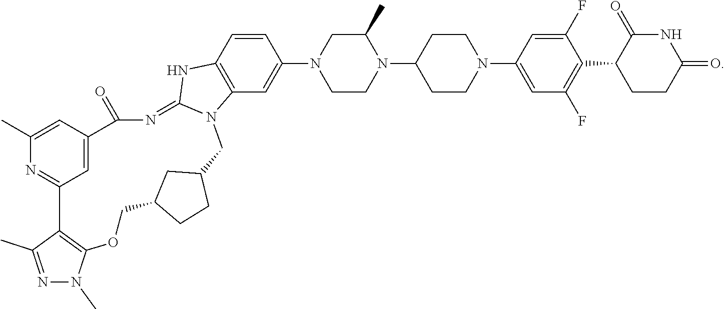

96. The compound of claim 77, wherein the compound is

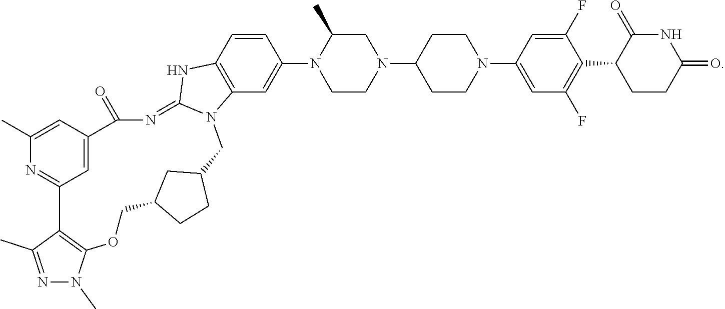

97. The compound of claim 77, wherein the compound is

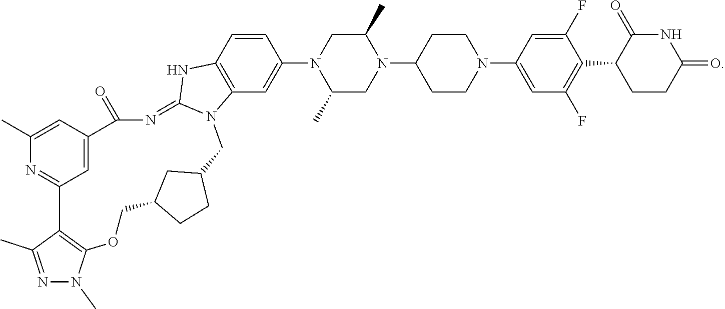

98. The compound of claim 77, wherein the compound is

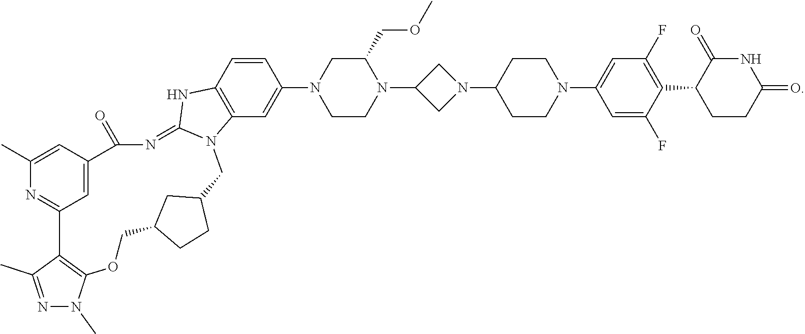

99. The compound of claim 77, wherein the compound is

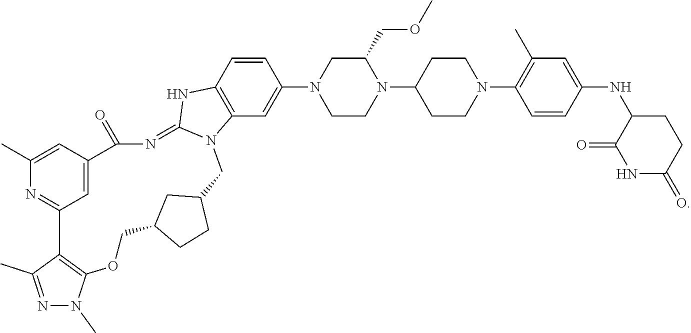

100. The compound of claim 77, wherein the compound is

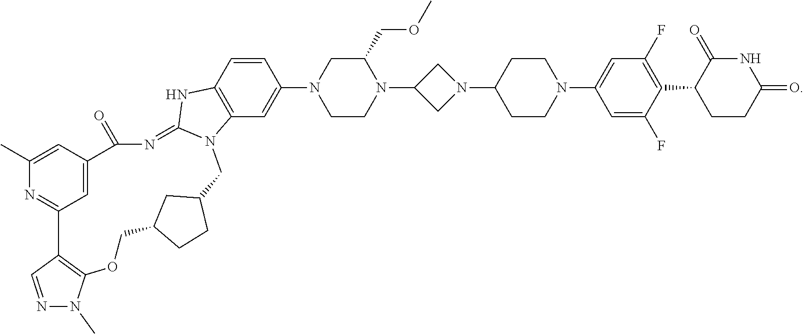

101. The compound of claim 77, wherein the compound is

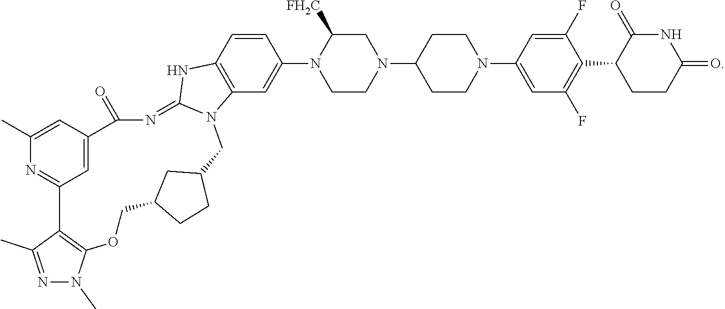

102. The compound of claim 77, wherein the compound is

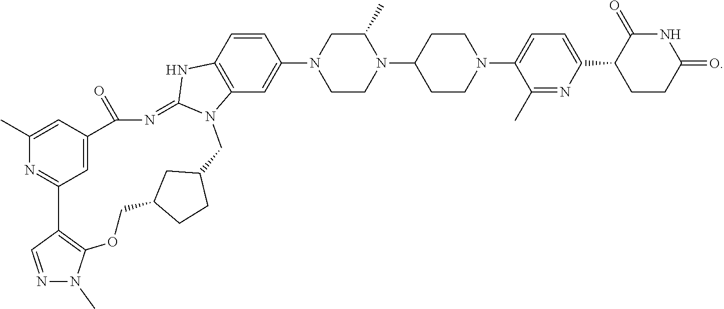

103. The method of claim 79, wherein the disease is selected from pancreatic cancer, breast cancer, glioblastoma multiforme, head and neck cancer, and non-small cell lung cancer.
